(19) United States
(12) Patent Application Publication
(10) Pub. No.: US 2026/0009009 A1
(43) Pub. Date: Jan. 8, 2026

(54) MODIFIED NUCLEASES

(71) Applicant: Celyntra Therapeutics SA, Mont-Saint-Guibert (BE)

(72) Inventors: Roland BAUMGARTNER, Angern an der March (AT); Tanya WARNECKE, Boulder, CO (US)

(21) Appl. No.: 18/289,723

(22) PCT Filed: May 6, 2022
(86) PCT No.: PCT/US2022/028208

§ 371 Date: Nov. 6, 2023

Related U.S. Application Data
(60) Provisional application No. 63/185,315, filed on May 6, 2021.
(60) Provisional application No. 63/315,483, filed on Mar. 1, 2022.

(51) Int. Cl. C12N 9/22; C12N 15/11

(52) U.S. Cl. CPC C12N 9/226; C12N 15/11; C07K 2319/09; C07K 2319/21; C12N 2310/20

(57) ABSTRACT

Provided herein are methods and compositions utilizing modified nucleases and/or other components, such as guide nucleic acids and donor templates, for use in a CRISPR system.

### CROSS-REFERENCE

This application is a continuation of PCT/US2022/028208, filed May 6, 2022, which claims priority to U.S. Provisional Application No. 63/185,315, filed May 6, 2021, and to U.S. Provisional Application No. 63/315,483, filed Mar. 1, 2022, both of which are incorporated herein by reference.

### SEQUENCE LISTING

The instant application contains a Sequence Listing which has been submitted electronically in .TXT file format and is hereby incorporated by reference in its entirety. Said. TXT copy, created on Jun. 16, 2023, is named P62036790WO-US_ST25.txt and is 1.550.589 bytes in size.

### BACKGROUND

Nucleic acid-guided nucleases have become important tools for research and genome engineering. The applicability of these tools can be limited by the sequence specificity requirements, expression, or delivery issues.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a diagram of MAD7 comprising one or more nuclear localization signals (NLS). FIG. 1 discloses “His6” as SEQ ID NO: 423.

FIG. 2 shows editing frequency at the DNMT1 locus in and post-transfection cell viability of T-cell leukemic cells following treatment comprising one or more guide nucleic acids complexed with MAD7 comprising one or more NLS.

FIG. 3 shows editing frequency at the DNMT1 locus in T-cell leukemic cells using multiple electroporation programs in combination with the SE electroporation buffer.

FIG. 4 shows editing frequency at the DNMT1 locus in T-cell leukemic cells using multiple electroporation programs in combination with the SF electroporation buffer.

FIG. 5 shows editing frequency at the DNMT1 locus in T-cell leukemic cells using multiple electroporation programs in combination with the SG electroporation buffer.

FIG. 6 shows editing frequency at the DNMT1 locus in T-cell leukemic cells using multiple electroporation programs.

FIG. 7 shows editing frequency by type at eight loci in T-cell leukemic cells using multiple guide nucleic acids complexed with MAD7 comprising one or more NLS.

FIG. 8 shows a comparison of editing efficiency between T-cell leukemic cells treated with MAD7 comprising one or more guide nucleic acids targeting the DNMT1 locus as compared to a control guide nucleic acid binned by editing frequency.

FIG. 9 shows editing frequency by PAM motif in T-cell leukemic cells using multiple guide nucleic acids complexed with MAD7 comprising one or more NLS.

FIG. 10A shows sequence logo plots for multiple guide nucleic acids binned by editing frequency in T-cell leukemic cells using when complexed with MAD7 comprising one or more NLS.

FIG. 10B shows nucleotide and dinucleotide frequency for multiple guide nucleic acids binned by editing frequency in T-cell leukemic cells using when complexed with MAD7 comprising one or more NLS.

FIG. 11 shows trinucleotide AAA or UUU frequency binned by editing frequency in T-cell leukemic cells following treatment with multiple guide nucleic acids complexed with MAD7 comprising one or more NLS.

FIG. 12 shows editing frequency for both INDELs and frameshift mutations at eight loci in T-cell leukemic cells following treatment with multiple guide nucleic acids complexed with MAD7 comprising one or more NLS.

FIG. 13 shows the correlation between INDEL frequency in the gNA validation experiment versus INDEL formation in the gNA screen experiment.

FIG. 14 shows the proportion of frameshift to INDELs at eight loci in T-cell leukemic cells following treatment with multiple guide nucleic acids complexed with MAD7 comprising one or more NLS.

FIG. 15 shows INDEL frequency for gNAs comprising representative spacer sequences complexed with MAD7 comprising one or more NLS in T-cell leukemic cells at predicted off-target sites. FIG. 15 discloses SEQ ID NOS 424-427, 427-429 and 429-454, respectively, in order of appearance.

FIG. 16 shows INDEL frequency for gNAs comprising representative spacer sequences complexed with MAD7 comprising one or more NLS in T-cell leukemic cells at predicted off-target sites. FIG. 16 discloses SEQ ID NOS 455-484, 453-454 and 485-487, respectively, in order of appearance.

FIG. 17 shows INDEL frequency at the AAVS1 locus in T-cell leukemic cells following treatment with a gNA:MAD7 complex.

FIG. 18 shows GFP insertion efficiency at the AAVS1 locus and cell viability following treatment for multiple primer constructs.

FIG. 19 shows GFP insertion efficiency at the AAVS1 locus with increasing concentrations of donor template (e.g., HDRT) and variable homology arm length.

FIG. 20 shows CAR insertion efficiency at the AAVS1 locus and cell viability with increasing concentrations of donor template and variable homology arm length.

FIG. 21 shows CAR insertion efficiency (A) at the AAVS1 locus and cell viability (B) in primary T-cells.

### DETAILED DESCRIPTION

CRISPR is an abbreviation of Clustered Regularly Interspaced Short Palindromic Repeats. In a palindromic repeat, the sequence of nucleotides is the same in both directions. Each of these palindromic repetitions is followed by short segments of spacer DNA. Small clusters of Cas (CRISPR-associated system) genes are located next to CRISPR sequences. The CRISPR/Cas system is a prokaryotic immune system that can confer resistance to foreign genetic elements such as those present within plasmids and phages providing the prokaryote a form of acquired immunity. RNA harboring a spacer sequence assists Cas (CRISPR-associated) proteins to recognize and cut exogenous DNA. CRISPR sequences are found in approximately 50% of bacterial genomes and nearly 90% of sequenced archaea has selected for efficient and robust metabolic and regulatory networks that prevent unnecessary metabolite biosynthesis and optimally distribute resources to maximize overall cellular fitness. The complexity of these networks with limited approaches to understand their structure and function and the ability to re-program cellular networks to modify these systems for a diverse range of applications has complicated advances in this space. Certain approaches to re-program cellular networks are directed to modifying single genes of complex pathways but as a consequence of modifying single genes, unwanted modifications to the genes or other genes can result, getting in the way of identifying changes necessary to achieve a sought-after endpoint as well as complicating the endpoint sought by the modification.

CRISPR-Cas driven genome editing and engineering has dramatically impacted biology and biotechnology in general. CRISPR-Cas editing systems require a polynucleotide guided nuclease, a guide nucleic acid (gNA) e.g. a guide RNA (gRNA)) that directs the nuclease to cut a specific region of the genome, and, optionally, a donor DNA cassette (also referred to herein as a donor template or editing sequence) that can be used to repair the cut dsDNA and thereby incorporate programmable edits at the site of interest. The earliest demonstrations and applications of CRISPR-Cas editing used Cas9 nucleases and associated gRNA. These systems have been used for gene editing in a broad range of species encompassing bacteria to higher order mammalian systems such as animals and in certain cases, humans. It is well established, however, that important editing parameters such as protospacer adjacent motif (PAM) specificity, editing efficiency, and off-target rates, among others, are species, loci, and nuclease dependent. There is increasing interest in identifying and rapidly characterizing novel nuclease systems that can be exploited to broaden and improve overall editing capabilities.

One version of the CRISPR/Cas system, CRISPR/Cas9, has been modified to provide useful tools for editing targeted genomes. By delivering the Cas9 nuclease complexed with a synthetic guide RNA (gRNA) into a cell, the cell's genome can be cut/edited at a predetermined location, allowing existing genes to be removed and/or new ones added. These systems are useful but have some important limitations regarding efficiency and accuracy of targeted editing, imprecise editing complications, as well as impediments when used for commercially relevant situations such as gene replacement. Therefore, a need exists for improved nucleic acid guided nuclease systems for directed and accurate editing with improved efficiency.

As used herein, the term “modulating” and “manipulating” of genome editing can mean an increase, a decrease, upregulation, downregulation, induction, a change in editing activity, a change in binding, a change cleavage or the like, of one or more of targeted genes or gene clusters of certain embodiments disclosed herein.

In certain embodiments of the present disclosure, there can be employed conventional molecular biology, microbiology, and recombinant DNA techniques within the skill of the art. Such techniques are explained fully in the literature and understood by those of skill in the art.

In other embodiments, primers used herein for preparation per conventional techniques can include sequencing primers and amplification primers. In some embodiments, plasmids and oligomers used in conventional techniques can include synthesized oligomers and oligomer cassettes.

In some embodiments disclosed herein, nucleic acid-guided nuclease systems and methods of use are provided. A nuclease system can include transcripts and other elements involved in the expression of an engineered nuclease disclosed herein, which can include sequences encoding a novel engineered nucleic acid-guided nuclease protein and a guide sequence (gRNA) or a novel gRNA as disclosed herein. In some embodiments, nucleic acid-guided nuclease systems can include at least one CRISPR-associated nucleic acid guided nuclease construct, the disclosure of which are provided herein. In other embodiments, nucleic acid-guided nuclease systems can include at least one known guide sequence (gRNA) or at least one novel gRNA, such as a single gRNA or a dual gRNA. In some embodiments, an engineered nucleic acid-guided nuclease of the instant invention can be used in systems for editing a gene of interest in humans or other species.

Bacterial and archaeal targetable nuclease systems have emerged as powerful tools for precision genome editing. However, naturally occurring nucleases have some limitations including expression and delivery challenges due to the nucleic acid sequence and protein size. In certain embodiments, novel engineered nucleic acid-guided nuclease constructs disclosed herein can be created for targeting of a targeted gene and/or increased efficiency and/or accuracy of targeted gene editing in a subject.

In accordance with these embodiments, it is known that Cas12a is a single RNA-guided CRISPR/Cas endonuclease capable of genome editing having differing features when compared to Cas9. In certain embodiments, a Cas12a-based system allow fast and reliable introduction of donor DNA into a genome. In addition, Cas12a broadens genome editing. CRISPR/Cas12a genome editing has been evaluated in human cells as well as other organisms including plants. Several features of the CRISPR/Cas12a system are different when compared to CRISPR/Cas9.

It is known that Cas12a nuclease recognizes T-rich protospacer adjacent motif (PAM) sequences (e.g. 5′-TTTN-3′ (AsCas12a, LbCas12a) and 5′-TTN-3′ (FnCas12a); whereas, the comparable sequence for SpCas9 is NGG. The PAM sequence of Cas12a is located at the 5′ end of the target DNA sequence, where it is at the 3′ end for Cas9. In addition, Cas12a is capable of cleaving DNA distal to its PAM around the +18/+23 position of the protospacer. This cleavage creates a staggered DNA overhang (e.g. sticky ends), whereas Cas9 cleaves close to its PAM after the 3′ position of the protospacer at both strands and creates blunt ends. In certain methods, creating altered recognition of nucleases can provide an improvement over Cas9 or Cas12a to improve accuracy. Further, Cas12a is guided by a single crRNA and does not require a tracrRNA, resulting in a shorter gRNA sequence than the sgRNA used by Cas9. Surprisingly, it has been found that the modified Cas12a nucleases provided herein can also function with a dual gRNA.

It is also known that Cas12a displays additional ribonuclease activity that functions in crRNA processing. Cas12a is used as an editing tool for different species (e.g. S. cerevisiae), allowing the use of an alternative PAM sequence compared with the one recognized by CRISPR/Cas9. Novel nucleases disclosed herein can further recognize the same or alternative PAM sequences. These novel nucleases can provide an alternative system for multiplex genome editing as compared with known multiplex approaches and can be used as an improved system in mammalian gene editing.

Well-known Cas12a protein-RNA complexes recognize a T-rich PAM and cleavage leads to a staggered DNA double-stranded break. Cas12a-type nuclease interacts with the pseudoknot structure formed by the 5′-handle of crRNA. A guide RNA segment, composed of a seed region and the 3′ terminus, possesses complementary binding sequences with the target DNA sequences. Cas12a type nucleases characterized to date have been demonstrated to work with a single gRNA and to process gRNA arrays. While Cas12a-type and Cas9 nuclease systems have proven highly impactful, neither system has been demonstrated to function as predictably as is desired to enable the full range of applications envisioned for gene-editing technologies.

In the current state, a range of efforts have attempted to engineer improved CRISPR editing systems having increased efficiency and accuracy, which have included engineering of the PAM specificity, stability, and sequence of the gRNA and-or the nuclease. For example, chemical modifications of CRISPR/Cas9 gRNA expected to increase gRNA stability was found to lead to a 3.8-fold higher indel frequencies in human cells. In addition, other studies included structure-guided mutagenesis of Cas12a and screened to identify variants with an increased range of recognized PAM sequences. These engineered AsCas12a recognized TYCV and TATV PAMs in addition to the established TTTV sequence, with enhanced activities in vitro and in tested human cells.

In certain embodiments, Cas12a-like nucleases and engineered gRNAs disclosed herein are contemplated for use in bacteria, and other prokaryotes. In certain embodiments, engineered designer nucleases are contemplated for use in eukaryotes such as yeast, mammals, e.g., human as well as of use in birds and fish, or cells derived from same.

In some embodiments, off-targeting rates for nuclease constructs disclosed herein can be reduced compared to a control, e.g., a native sequence, for improved editing. Off-targeting rates can be readily tested.

In some embodiments, nuclease constructs disclosed herein can share conserved encoded motifs of known nucleases. In other embodiments, nuclease constructs disclosed herein do not share conserved encoded peptide motifs with known nucleases. In preferred embodiments, provided herein are compositions, methods, and/or kits wherein the CRISPR nuclease comprises a Type V nuclease. In certain embodiments, provided herein are compositions, methods, and/or kits wherein the CRISPR nuclease comprises a Type V-A, V-B, V-C, V-D, or V-E CRISPR nuclease. In certain embodiments, provided herein are compositions, methods, and/or kits wherein the CRISPR nuclease comprises a Type V-A nuclease. Naturally occurring type V-A CRISPR nucleases comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. These CRISPR nucleases cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

In certain embodiments, a type V-A CRISPR nuclease comprises Cpf1. Cpf1 proteins are known in the art and are described, e.g., in U.S. Pat. Nos. 9,790,490 and 10,113,179. Cpf1 orthologs can be found in various bacterial and archaeal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2), Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Eubacterium eligens, Leptospira inadai, Porphyromonas macacae, Prevotella bryantii, Proteocatella sphenisci, Anaerovibrio sp. RM50, Moraxella caprae, Lachnospiraceae bacterium COE1, or Eubacterium coprostanoligenes.

In certain embodiments, a type V-A CRISPR nuclease comprises AsCpf1 or a variant thereof. In certain embodiments, a type V-A CRISPR nucleases comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 3 of International (PCT) Application Publication No. WO 2021/158918. In certain embodiments, a type V-A CRISPR nucleases comprises the amino acid sequence set forth in SEQ ID NO: 3 of International (PCT) Application Publication No. WO 2021/158918.

In certain embodiments, a type V-A CRISPR nuclease comprises LbCpf1 or a variant thereof. In certain embodiments, a type V-A CRISPR nucleases comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 4 of International (PCT) Application Publication No. WO 2021158918. In certain embodiments, a type V-A Cas protein comprises the amino acid sequence set forth in SEQ ID NO: 4 of International (PCT) Application Publication No. WO 2021/158918.

In certain embodiments, a type V-A CRISPR nuclease comprises FnCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 5 of International (PCT) Application Publication No. WO 2021158918. In certain embodiments, a type V-A Cas protein comprises the amino acid sequence set forth in SEQ ID NO: 5 of International (PCT) Application Publication No. WO 2021/158918.

In certain embodiments, a type V-A CRISPR nuclease comprises Prevotella bryantii Cpf1 (PbCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 6 of International (PCT) Application Publication No. WO 2021/158918. In certain embodiments, a type V-A Cas protein comprises the amino acid sequence set forth in SEQ ID NO: 6 of International (PCT) Application Publication No. WO 2021/158918.

In certain embodiments, a type V-A CRISPR nuclease comprises Proteocatella sphenisci Cpf1 (PsCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 7 of International (PCT) Application Publication No. WO 2021158918. In certain embodiments, a type V-A Cas protein comprises the amino acid sequence set forth in SEQ ID NO: 7 of International (PCT) Application Publication No. WO 2021/158918.

In certain embodiments, a type V-A CRISPR nuclease comprises Anaerovibrio sp. RM50 Cpf1 (As2Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 8 of International (PCT) Application Publication No. WO 2021158918. In certain embodiments, a type V-A Cas protein comprises the amino acid sequence set forth in SEQ ID NO: 8 of International (PCT) Application Publication No. WO 2021/158918.

In certain embodiments, a type V-A CRISPR nuclease comprises Moraxella caprae Cpf1 (McCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 9 of International (PCT) Application Publication No. WO 2021/158918. In certain embodiments, a type V-A Cas protein comprises the amino acid sequence set forth in SEQ ID NO: 9 of International (PCT) Application Publication No. WO 2021/158918.

In certain embodiments, a type V-A CRISPR nuclease comprises Lachnospiraceae bacterium COE1 Cpf1 (Lb3Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 10 of International (PCT) Application Publication No. WO 2021158918. In certain embodiments, a type V-A Cas protein comprises the amino acid sequence set forth in SEQ ID NO: 10 of International (PCT) Application Publication No. WO 2021/158918.

In certain embodiments, a type V-A CRISPR nuclease comprises Eubacterium coprostanoligenes Cpf1 (EcCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 11 of International (PCT) Application Publication No. WO 2021158918. In certain embodiments, a type V-A Cas protein comprises the amino acid sequence set forth in SEQ ID NO: 11 of International (PCT) Application Publication No. WO 2021/158918.

In certain embodiments, a type V-A CRISPR nuclease is not Cpf1. In certain embodiments, a type V-A CRISPR nuclease is not AsCpf1.

In certain embodiments, a type V-A CRISPR nuclease comprises a Type V-A nuclease described in U.S. Pat. No. 9,982,279.

In certain embodiments, a Type VA CRISPR nuclease polypeptide used in compositions and methods herein can be represented by a polypeptide that includes a sequence that has at least 60, 70, 80, 85, 90, 95, 96, 97, 98, 99, or 100% sequence identity, preferably at least 80%, more preferably at least 90%, even more preferably at least 95%, still more preferably at least 98% sequence identity with SEQ ID NO: 1 SEQ ID NO: 1 wherein the Type VA CRISPR nuclease polypeptide further comprises at least one, two, three, four, five or six nuclear localization sequences (NLS), each of which can be at or near the amino end or carboxy end of the CRISPR nuclease polypeptide; and/or one or more purification tags; in addition, a cleavage sequence can be provided to remove portions of a protopeptide. As used herein, the term “at or near” an N-terminus or a C-terminus includes where the nearest amino acid of the NLS to the N- or C-terminus is within 300 amino acids, in some cases within 200 amino acids, from the N- or C-terminus of the polypeptide (e.g., a core polypeptide such as one of the CRISPR nucleases described herein, to which the NLS or NLSs is attached). In certain emobidments, a Type V CRISPR nuclease polypeptide, e.g., Type Va CRISPR polypeptide, comprises two, three, four, or five NLSs, each of which are at or near the N-terminus or the C-terminus of the polypeptide, in preferred embodiments the NLSs are at or near the N-terminus. In certain embodiments, a CRISPR nuclease polypeptide, including one or more NLSs and, in some cases, a purification tag and/or a cleavage site, comprises a sequence at least 60, 70, 80, 85, 90, 95, 98, 99%, or 100%, identical, preferably at least 80%, more preferably at least 90%, even more preferably at least 95%, still more preferably at least 98% identical to any one of SEQ ID NOs: 109-112. In certain embodiments, a Type V, e.g., VA CRISPR nuclease polypeptide comprises at least 1-30, 1-20, 1-15, 1-10, 1-9, 1-8, 1-7, 1-6, 1-5, 2-30, 2-20, 2-15, 2-10, 2-9, 2-8, 2-7, 2-6, 2-5, 3-30, 3-20, 3-15, 3-10, 3-9, 3-8, 3-7, 3-6, or 3-5, preferably 1-10, more preferably 2-10, even more preferably 3-10 NLSs, each of which is at or near the N-terminus or the C-terminus of the polypeptide, in preferred embodiments at or near the N-terminus. In certain embodiments, at least two, or at least three, of the NLSs have different mechanisms, that is, different mechanisms by which they localize an attached polypeptide to a nucleus. Such mechanisms are well-known in the art; see, e.g., Lu et al. Cell Commun Signal (2021) 19:60 https://doi.org/10.1186/s12964-021-00741-y. Suitable NLS, purification tag, and cleavage site sequences can be as described elsewhere herein, e.g., in sections labled Nuclear Localization Signals, Purification Tags, and Cleavage Sites.

SEQ ID NO: 1
MNNGTNNFQNFIGISSLQKTLRNALIPTETTQQFIVKNGIIKEDE
LRGENRQILKDIMDDYYRGFISETLSSIDDIDWTSLFEKMEIQLK
NGDNKDTLIKEQTEYRKAIHKKFANDDRFKNMFSAKLISDILPEF
VIHNNNYSASEKEEKTQVIKLFSRFATSFKDYFKNRANCFSADDI
SSSSCHRIVNDNAEIFFSNALVYRRIVKSLSNDDINKISGDMKDS
LKEMSLEEIYSYEKYGEFITQEGISFYNDICGKVNSFMNLYCQKN
KENKNLYKLQKLHKQILCIADTSYEVPYKFESDEEVYQSVNGFLD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVFMTEELVDKDNNFYAELEEIYDEIY
PVISLYNLVRNYVTQKPYSTKKIKLNFGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDFDI
TFCHDLIDYFKNCIAIHPEWKNFGFDFSDTSTYEDISGFYREVEL
QGYKIDWTYISEKDIDLLQEKGQLYLFQIYNKDFSKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYFLHMPITINFKANKTG
FINDRILQYIAKEKDLHVIGIDRGERNLIYVSVIDTCGNIVEQKS
FNIVNGYDYQIKLKQQEGARQIARKEWKEIGKIKEIKEGYLSLVI
HEISKMVIKYNAIIAMEDLSYGFKKGRFKVERQVYQKFETMLINK
LNYLVFKDISITENGGLLKGYQLTYIPDKLKNVGHQCGCIFYVPA
AYTSKIDPTTGFVNIFKFKDLTVDAKREFIKKFDSIRYDSEKNLF
CFTFDYNNFITQNTVMSKSSWSVYTYGVRIKRRFVNGRFSNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWFDFIQNK
RYL

Nucleotide sequences coding for SEQ ID NO: 1 can include sequences with less than 99, 95, 90, 85, 80, 75, 70, 65, 60, 55, 50, 45, or 40% sequence identity with SEQ ID NO: 22, in preferred embodiments less than 75% sequence identity. In certain embodiments, a nucleotide sequence coding for SEQ ID NO: 1 can also include nucleic acid sequences coding for one or more NLS at the N-terminus and/or C-terminus, as described herein, and/or a tag such as a purification tag at the N-terminus, as described herein. In certain embodiments, provided herein are compositions comprising a first polynucleotide coding for a polypeptide comprising a nucleic acid-guided nuclease comprising a CRISPR Type V nuclease polypeptide, wherein the polynucleotide has less than 75% sequence identity to SEQ ID NO: 22, such as wherein the nuclease polypeptide comprises at least 1, 2, 3, 4, or 5 NLSs, wherein each of the NLSs is at or near the N-terminus or the C-terminus of the nuclease polypeptide. NLSs can be any of those described herein. The first polynucleotide can comprise a sequence coding for a purification tag, such as a purification tag described herein, and/or cleavage site, such as a cleavage site described herein. In certain embodiments the first polynucleotide codes for a polypeptide comprising a sequence at least 60, 70, 80, 85, 90, 95, 98, 99%, or 100%, identical, preferably at least 80%, more preferably at least 90%, even more preferably at least 95%, still more preferably at least 98% identical to any one of SEQ ID NOs: 109-112, such as SEQ ID NO: 109, or SEQ ID NO: 110, or SEQ ID NO: 111, or SEQ ID NO: 112. the first polynucleotide comprises a sequence at least 50, 60, 70, 80, 90, 95, 97, or 99% identical, or 100% identical, preferably at least 80%, more preferably at least 90%, even more preferably at least 95%, still more preferably at least 98% identical to SEQ ID NO: 113. In certain embodiment the composition further comprises a second polynucleotide coding for a gNA or portion thereof, wherein the gNA, e.g., gRNA, comprises a spacer sequence that targets a target nucleotide sequence within a polynucleotide, or a polynuclotide coding for the gNA, e.g., gRNA, wherein the gNA, e.g., gRNA is compatible with the Type V CRISPR nuclease. In certain embodiments the first and second polynucleotides are the same. The composition can further comprise a third polynucleotide comprising a donor template. In certain embodiments, provided is a vector comprising one of the polynucleotide compositions of this paragraph. In certain embodiments, provided is a cell comprising one of the polynucleotide compositions of this paragraph, e.g., a human cell, such as an immune cell, for example a T cell, or a stem cell, such as an iPSC. In certain embodiments, provided is a method comprising inserting any one of the polynucleotide compositions of this paragraph into a cell. In certain embodiments inserting the composition comprises electroporation.

SEQ ID NO: 22:
ATGAACAACGGCACAAATAATTTTCAGAACTTCATCGGGATCTCA
AGTTTGCAGAAAACGCTGCGCAATGCTCTGATCCCCACGGAAACC
ACGCAACAGTTCATCGTCAAGAACGGAATAATTAAAGAAGATGAG
TTACGTGGCGAGAACCGCCAGATTCTGAAAGATATCATGGATGAC
TACTACCGCGGATTCATCTCTGAGACTCTGAGTTCTATTGATGAC
ATAGATTGGACTAGCCTGTTCGAAAAAATGGAAATTCAGCTGAAA
AATGGTGATAATAAAGATACCTTAATTAAGGAACAGACAGAGTAT
CGGAAAGCAATCCATAAAAAATTTGCGAACGACGATCGGTTTAAG
AACATGTTTAGCGCCAAACTGATTAGTGACATATTACCTGAATTT
GTCATCCACAACAATAATTATTCGGCATCAGAGAAAGAGGAAAAA
ACCCAGGTGATAAAATTGTTTTCGCGCTTTGCGACTAGCTTTAAA
GATTACTTCAAGAACCGTGCAAATTGCTTTTCAGCGGACGATATT
TCATCAAGCAGCTGCCATCGCATCGTCAACGACAATGCAGAGATA
TTCTTTTCAAATGCGCTGGTCTACCGCCGGATCGTAAAATCGCTG
AGCAATGACGATATCAACAAAATTTCGGGCGATATGAAAGATTCA
TTAAAAGAAATGAGTCTGGAAGAAATATATTCTTACGAGAAGTAT
GGGGAATTTATTACCCAGGAAGGCATTAGCTTCTATAATGATATC
TGTGGGAAAGTGAATTCTTTTATGAACCTGTATTGTCAGAAAAAT
AAAGAAAACAAAAATTTATACAAACTTCAGAAACTTCACAAACAG
ATTCTATGCATTGCGGACACTAGCTATGAGGTCCCGTATAAATTT
GAAAGTGACGAGGAAGTGTACCAATCAGTTAACGGCTTCCTTGAT
AACATTAGCAGCAAACATATAGTCGAAAGATTACGCAAAATCGGC
GATAACTATAACGGCTACAACCTGGATAAAATTTATATCGTGTCC
AAATTTTACGAGAGCGTTAGCCAAAAAACCTACCGCGACTGGGAA
ACAATTAATACCGCCCTCGAAATTCATTACAATAATATCTTGCCG
GGTAACGGTAAAAGTAAAGCCGACAAAGTAAAAAAAGCGGTTAAG
AATGATTTACAGAAATCCATCACCGAAATAAATGAACTAGTGTCA
AACTATAAGCTGTGCAGTGACGACAACATCAAAGCGGAGACTTAT
ATACATGAGATTAGCCATATCTTGAATAACTTTGAAGCACAGGAA
TTGAAATACAATCCGGAAATTCACCTAGTTGAATCCGAGCTCAAA
GCGAGTGAGCTTAAAAACGTGCTGGACGTGATCATGAATGCGTTT
CATTGGTGTTCGGTTTTTATGACTGAGGAACTTGTTGATAAAGAC
AACAATTTTTATGCGGAACTGGAGGAGATTTACGATGAAATTTAT
CCAGTAATTAGTCTGTACAACCTGGTTCGTAACTACGTTACCCAG
AAACCGTACAGCACGAAAAAGATTAAATTGAACTTTGGAATACCG
ACGTTAGCAGACGGTTGGTCAAAGTCCAAAGAGTATTCTAATAAC
GCTATCATACTGATGCGCGACAATCTGTATTATCTGGGCATCTTT
AATGCGAAGAATAAACCGGACAAGAAGATTATCGAGGGTAATACG
TCAGAAAATAAGGGTGACTACAAAAAGATGATTTATAATTTGCTC
CCGGGTCCCAACAAAATGATCCCGAAAGTTTTCTTGAGCAGCAAG
ACGGGGGTGGAAACGTATAAACCGAGCGCCTATATCCTAGAGGGG
TATAAACAGAATAAACATATCAAGTCTTCAAAAGACTTTGATATC
ACTTTCTGTCATGATCTGATCGACTACTTCAAAAACTGTATTGCA
ATTCATCCCGAGTGGAAAAACTTCGGTTTTGATTTTAGCGACACC
AGTACTTATGAAGACATTTCCGGGTTTTATCGTGAGGTAGAGTTA
CAAGGTTACAAGATTGATTGGACATACATTAGCGAAAAAGACATT
GATCTGCTGCAGGAAAAAGGTCAACTGTATCTGTTCCAGATATAT
AACAAAGATTTTTCGAAAAAATCAACCGGGAATGACAACCTTCAC
ACCATGTACCTGAAAAATCTTTTCTCAGAAGAAAATCTTAAGGAT
ATCGTCCTGAAACTTAACGGCGAAGCGGAAATCTTCTTCAGGAAG
AGCAGCATAAAGAACCCAATCATTCATAAAAAAGGCTCGATTTTA
GTCAACCGTACCTACGAAGCAGAAGAAAAAGACCAGTTTGGCAAC
ATTCAAATTGTGCGTAAAAATATTCCGGAAAACATTTATCAGGAG
CTGTACAAATACTTCAACGATAAAAGCGACAAAGAGCTGTCTGAT
GAAGCAGCCAAACTGAAGAATGTAGTGGGACACCACGAGGCAGCG
ACGAATATAGTCAAGGACTATCGCTACACGTATGATAAATACTTC
CTTCATATGCCTATTACGATCAATTTCAAAGCCAATAAAACGGGT
TTTATTAATGATAGGATCTTACAGTATATCGCTAAAGAAAAAGAC
TTACATGTGATCGGCATTGATCGGGGCGAGCGTAACCTGATCTAC
GTGTCCGTGATTGATACTTGTGGTAATATAGTTGAACAGAAAAGC
TTTAACATTGTAAACGGCTACGACTATCAGATAAAACTGAAACAA
CAGGAGGGCGCTAGACAGATTGCGCGGAAAGAATGGAAAGAAATT
GGTAAAATTAAAGAGATCAAAGAGGGCTACCTGAGCTTAGTAATC
CACGAGATCTCTAAAATGGTAATCAAATACAATGCAATTATAGCG
ATGGAGGATTTGTCTTATGGTTTTAAAAAAGGGCGCTTTAAGGTC
GAACGGCAAGTTTACCAGAAATTTGAAACCATGCTCATCAATAAA
CTCAACTATCTGGTATTTAAAGATATTTCGATTACCGAGAATGGC
GGTCTCCTGAAAGGTTATCAGCTGACATACATTCCTGATAAACTT
AAAAACGTGGGTCATCAGTGCGGCTGCATTTTTTATGTGCCTGCT
GCATACACGAGCAAAATTGATCCGACCACCGGCTTTGTGAATATC
TTTAAATTTAAAGACCTGACAGTGGACGCAAAACGTGAATTCATT
AAAAAATTTGACTCAATTCGTTATGACAGTGAAAAAAATCTGTTC
TGCTTTACATTTGACTACAATAACTTTATTACGCAAAACACGGTC
ATGAGCAAATCATCGTGGAGTGTGTATACATACGGCGTGCGCATC
AAACGTCGCTTTGTGAACGGCCGCTTCTCAAACGAAAGTGATACC
ATTGACATAACCAAAGATATGGAGAAAACGTTGGAAATGACGGAC
ATTAACTGGCGCGATGGCCACGATCTTCGTCAAGACATTATAGAT
TATGAAATTGTTCAGCACATATTCGAAATTTTCCGTTTAACAGTG
CAAATGCGTAACTCCTTGTCTGAACTGGAGGACCGTGATTACGAT
CGTCTCATTTCACCTGTACTGAACGAAAATAACATTTTTTATGAC
AGCGCGAAAGCGGGGGATGCACTTCCTAAGGATGCCGATGCAAAT
GGTGCGTATTGTATTGCATTAAAAGGGTTATATGAAATTAAACAA
ATTACCGAAAATTGGAAAGAAGATGGTAAATTTTCGCGCGATAAA
CTCAAAATCAGCAATAAAGATTGGTTCGACTTTATCCAGAATAAG
CGCTATCTCTAA

Exemplary nucleotide sequences coding for SEQ ID NO: 1 can include, e.g., SEQ ID NOs: 23-42:

SEQ ID NO: 23
ATGAACAACGGAACAAATAATTTTCAGAACTTTATTGGGATCAGTTCGCTTCAGAAAACG
CTTCGTAATGCTCTGATTCCCACAGAAACCACTCAGCAGTTTATCGTAAAGAATGGCATT
ATCAAGGAGGATGAATTACGCGGCGAGAACCGCCAAATCTTAAAAGATATCATGGACGAC
TACTACCGCGGTTTCATTAGCGAAACTCTTAGTTCAATTGACGACATTGACTGGACGTCC
TTGTTCGAAAAGATGGAGATTCAATTAAAGAACGGTGATAACAAGGATACGTTGATTAAA
GAACAGACGGAGTACCGTAAGGCTATCCACAAAAAATTTGCAAACGACGACCGCTTTAAA
AATATGTTTAGCGCAAAATTAATCTCCGACATCCTGCCTGAATTCGTCATCCATAACAAT
AACTATAGCGCCTCGGAAAAAGAAGAAAAAACGCAGGTTATTAAACTTTTCTCGCGCTTT
GCAACAAGCTTTAAGGATTACTTCAAAAATCGCGCCAATTGTTTTTCAGCCGACGACATT
AGCTCCAGTTCCTGCCACCGTATTGTGAATGACAACGCTGAGATTTTTTTTTCCAATGCG
CTGGTTTATCGTCGTATTGTTAAGAGCCTTAGTAACGACGACATTAATAAAATTAGCGGT
GATATGAAGGATAGCTTGAAAGAAATGAGTCTGGAAGAGATCTATAGTTACGAGAAGTAC
GGCGAATTTATTACCCAGGAGGGCATTTCATTTTACAATGATATCTGTGGAAAAGTCAAC
TCCTTTATGAACTTGTATTGCCAAAAGAATAAAGAAAACAAAAACCTGTACAAACTGCAA
AAGTTACACAAGCAGATTTTGTGTATCGCAGACACGTCATACGAAGTACCGTACAAGTTT
GAGTCCGATGAAGAAGTGTACCAAAGCGTTAATGGCTTTTTGGATAACATTTCGAGCAAA
CATATCGTAGAGCGTTTGCGTAAGATTGGTGATAATTACAACGGTTACAATTTAGACAAA
ATCTATATCGTCTCTAAGTTTTACGAAAGTGTTTCTCAGAAAACTTACCGCGATTGGGAG
ACGATCAACACTGCGCTGGAGATTCATTACAATAATATCCTTCCAGGTAACGGTAAAAGC
AAAGCTGATAAGGTGAAAAAGGCGGTTAAAAATGACCTTCAAAAGTCTATCACAGAAATC
AACGAATTGGTCAGCAATTATAAGCTTTGCAGTGACGATAACATTAAGGCCGAGACTTAC
ATCCATGAGATCTCTCACATTCTTAATAATTTTGAAGCGCAAGAGCTGAAATACAATCCT
GAAATCCATCTGGTCGAAAGTGAATTAAAAGCCTCCGAATTAAAAAATGTCTTGGACGTG
ATCATGAATGCGTTCCATTGGTGCTCAGTTTTTATGACGGAAGAGTTGGTGGACAAAGAC
AACAATTTTTACGCCGAGCTTGAGGAAATTTACGACGAAATTTACCCCGTTATTTCGTTA
TACAACCTTGTGCGTAATTACGTTACACAAAAGCCCTATTCGACAAAGAAAATCAAGTTA
AATTTCGGGATTCCCACATTAGCTGATGGATGGTCCAAATCCAAAGAATACTCGAATAAC
GCTATCATCCTTATGCGTGATAATTTGTACTACTTAGGCATCTTCAATGCGAAGAACAAA
CCTGACAAGAAAATTATCGAAGGAAACACTTCGGAGAACAAAGGTGATTATAAAAAGATG
ATCTACAACTTGCTTCCCGGGCCAAACAAAATGATTCCCAAGGTATTTTTGAGTTCTAAA
ACCGGTGTCGAAACTTACAAACCAAGTGCTTATATTTTGGAAGGATACAAACAGAACAAA
CATATCAAGTCTTCGAAAGACTTCGATATTACGTTCTGCCACGATCTGATCGATTACTTC
AAGAACTGTATTGCTATTCACCCCGAGTGGAAGAACTTTGGATTTGATTTCTCCGACACG
TCCACTTATGAAGATATCTCTGGCTTCTATCGCGAGGTTGAATTACAAGGGTATAAGATT
GACTGGACTTATATTTCGGAGAAGGATATCGATCTTTTGCAAGAAAAAGGGCAACTTTAT
TTATTTCAGATCTATAACAAGGACTTTTCAAAAAAGAGCACTGGAAATGACAATCTGCAT
ACCATGTACCTTAAGAACCTGTTCTCGGAAGAGAACCTGAAGGACATTGTACTTAAACTG
AATGGAGAGGCAGAGATCTTCTTTCGCAAATCAAGCATTAAGAACCCAATTATTCACAAA
AAGGGGAGTATCTTAGTAAATCGCACATATGAGGCTGAGGAAAAAGATCAGTTTGGTAAC
ATTCAGATCGTGCGTAAGAACATTCCTGAAAATATCTATCAGGAACTTTATAAGTATTTC
AACGATAAAAGTGATAAAGAGCTGAGTGACGAAGCGGCTAAACTTAAGAATGTTGTGGGA
CACCATGAGGCAGCAACCAATATTGTGAAGGATTATCGCTATACGTACGACAAATACTTT
TTACACATGCCCATCACTATTAATTTTAAAGCTAATAAGACTGGCTTCATTAACGATCGC
ATCCTGCAGTACATTGCTAAGGAAAAGGATCTTCACGTTATCGGTATCGATCGCGGGGAG
CGTAATCTTATCTACGTCTCTGTCATTGACACGTGTGGCAATATTGTGGAGCAAAAGTCC
TTCAATATTGTTAACGGCTATGACTATCAGATTAAATTGAAACAGCAGGAAGGTGCGCGT
CAGATTGCCCGCAAGGAATGGAAGGAAATTGGCAAGATCAAAGAAATTAAGGAGGGCTAC
TTAAGCTTAGTAATTCACGAAATTAGTAAAATGGTTATCAAATACAACGCCATCATCGCG
ATGGAGGATCTTTCGTACGGGTTTAAGAAAGGTCGTTTTAAAGTGGAGCGTCAGGTGTAC
CAGAAATTTGAAACTATGCTTATTAACAAACTTAACTACCTGGTTTTCAAGGATATCAGT
ATTACTGAAAACGGGGGGCTGTTAAAAGGGTATCAATTAACTTACATTCCAGACAAATTA
AAGAACGTTGGACATCAGTGTGGCTGCATTTTTTATGTACCAGCTGCATACACTTCAAAG
ATCGATCCTACGACTGGGTTCGTGAACATTTTTAAGTTTAAAGACTTGACGGTAGATGCC
AAGCGCGAATTCATCAAGAAATTCGACAGCATTCGCTACGACTCTGAGAAAAATCTTTTC
TGTTTCACATTCGATTATAACAATTTCATTACGCAGAACACAGTAATGTCCAAGTCTTCT
TGGAGTGTTTATACATATGGTGTCCGCATTAAGCGCCGTTTCGTCAACGGCCGCTTCAGT
AATGAGAGCGATACTATTGACATCACAAAAGACATGGAAAAAACACTGGAAATGACCGAC
ATCAATTGGCGTGACGGCCATGACTTACGTCAGGATATCATTGATTATGAGATCGTTCAA
CACATCTTCGAAATCTTTCGCTTGACTGTTCAAATGCGCAATTCCTTGTCGGAATTGGAG
GACCGTGATTATGACCGCTTAATTTCCCCCGTCTTAAATGAAAACAATATTTTTTATGAC
TCTGCAAAAGCTGGAGATGCTCTGCCGAAAGACGCCGATGCAAATGGGGCATATTGCATT
GCTTTAAAGGGGCTTTACGAGATCAAGCAAATCACCGAAAACTGGAAAGAGGATGGAAAG
TTTTCGCGTGATAAACTGAAGATCTCTAACAAAGACTGGTTCGACTTTATCCAGAACAAG
CGTTATTT
SEQ ID NO: 24
ATGAACAACGGCACCAATAACTTCCAAAACTTCATCGGGATCTCTAGCCTTCAGAAGACG
CTTCGCAATGCTCTTATCCCAACTGAGACCACTCAACAATTTATTGTGAAGAATGGAATT
ATTAAAGAGGACGAACTGCGTGGCGAGAATCGTCAGATCTTAAAGGACATTATGGATGAT
TATTACCGTGGATTCATCTCCGAAACATTATCGTCGATCGATGATATCGATTGGACTTCT
CTGTTCGAGAAAATGGAAATTCAATTGAAAAACGGAGATAATAAAGATACGCTTATCAAA
GAACAGACGGAATATCGTAAAGCGATTCATAAGAAATTCGCAAATGACGATCGTTTCAAA
AATATGTTCAGTGCCAAGCTTATTTCGGACATTTTACCTGAATTTGTAATTCATAATAAT
AACTACTCAGCAAGTGAGAAGGAGGAGAAAACCCAAGTTATTAAACTGTTCTCTCGTTTC
GCAACGTCCTTTAAAGATTACTTTAAAAACCGCGCGAATTGCTTTAGCGCTGACGACATT
TCCAGCTCATCCTGTCATCGCATCGTAAACGACAATGCGGAAATCTTCTTCAGCAACGCC
CTGGTTTACCGCCGCATCGTCAAAAGCTTATCGAATGACGACATCAATAAGATCTCAGGA
GATATGAAGGACTCGCTTAAGGAGATGTCTCTGGAGGAAATTTATAGTTACGAAAAGTAT
GGAGAGTTCATTACCCAGGAGGGAATCTCGTTCTACAATGACATTTGCGGGAAGGTGAAC
TCCTTCATGAACTTATACTGCCAGAAAAACAAAGAGAACAAAAATCTGTATAAATTGCAG
AAATTACATAAACAGATTCTTTGTATTGCTGACACTTCCTACGAAGTACCCTATAAATTC
GAGTCAGATGAAGAAGTATACCAGTCCGTGAACGGATTTCTGGACAATATCTCCTCAAAA
CACATCGTGGAACGCTTACGTAAAATTGGCGATAATTATAATGGTTACAATCTTGACAAA
ATTTATATCGTATCTAAATTTTACGAGAGTGTGAGCCAAAAGACCTACCGCGACTGGGAG
ACCATCAACACAGCTTTAGAAATTCACTATAATAATATCTTACCCGGCAATGGTAAGAGC
AAGGCTGACAAGGTAAAAAAGGCCGTCAAGAATGATTTGCAGAAATCTATTACAGAAATT
AATGAGTTAGTCTCCAACTATAAGCTTTGTTCCGACGATAACATCAAAGCTGAGACATAT
ATTCATGAGATTAGTCACATTCTTAACAACTTCGAGGCCCAGGAACTTAAGTACAATCCT
GAAATTCATCTTGTCGAGTCTGAGCTGAAAGCTAGTGAATTGAAAAATGTTTTAGACGTT
ATTATGAACGCATTCCACTGGTGCTCTGTGTTTATGACAGAAGAACTGGTCGACAAGGAC
AATAACTTCTATGCCGAACTTGAGGAAATCTACGATGAAATTTACCCTGTAATCTCCTTG
TATAATCTTGTACGTAATTACGTCACTCAAAAACCTTACAGCACGAAAAAAATTAAATTG
AACTTCGGGATTCCTACACTTGCCGACGGGTGGTCTAAATCCAAGGAATATAGCAACAAT
GCCATTATTTTAATGCGCGACAATCTTTACTATTTAGGAATTTTTAACGCTAAGAACAAG
CCCGATAAAAAGATTATTGAAGGAAACACGTCTGAAAATAAGGGCGACTACAAAAAGATG
ATTTATAACCTTTTGCCCGGTCCAAACAAAATGATCCCAAAGGTATTCCTGTCATCCAAA
ACAGGGGTTGAGACATATAAGCCCAGCGCATATATTCTGGAAGGATACAAACAGAATAAA
CATATCAAAAGCAGCAAAGATTTTGACATTACTTTTTGCCACGATTTAATCGACTACTTC
AAAAACTGTATCGCTATCCACCCTGAATGGAAGAATTTCGGATTTGATTTCTCAGATACA
AGTACGTATGAGGATATCAGCGGTTTCTATCGCGAAGTTGAACTTCAAGGGTATAAAATT
GACTGGACCTACATTAGTGAGAAGGACATCGACCTGTTACAGGAAAAAGGCCAATTGTAC
TTGTTTCAGATCTACAATAAGGATTTCTCAAAAAAATCGACCGGCAATGATAACTTGCAC
ACCATGTACCTGAAGAACCTTTTTTCGGAGGAAAACCTTAAAGACATTGTCCTGAAGTTG
AATGGAGAAGCGGAGATTTTCTTTCGTAAGTCTTCCATTAAAAATCCAATTATTCATAAG
AAGGGCAGCATCCTTGTGAACCGTACGTACGAGGCGGAAGAGAAGGACCAATTCGGTAAC
ATTCAAATCGTCCGCAAGAACATCCCTGAAAATATTTATCAGGAGCTTTACAAGTATTTC
AATGATAAGTCCGACAAGGAATTATCAGATGAGGCTGCGAAGTTGAAAAATGTTGTTGGT
CATCACGAGGCGGCGACGAATATTGTAAAGGATTATCGCTACACTTATGACAAGTACTTT
CTGCACATGCCGATCACCATTAATTTCAAGGCGAACAAAACAGGATTTATTAATGACCGC
ATCTTACAATACATTGCCAAAGAAAAGGACTTACACGTTATTGGCATTGATCGTGGAGAA
CGCAACTTAATCTACGTAAGCGTTATTGACACTTGCGGGAATATCGTAGAACAAAAGAGC
TTCAACATCGTGAATGGTTACGATTACCAGATCAAGCTTAAGCAGCAGGAGGGAGCGCGC
CAGATCGCGCGCAAGGAATGGAAGGAGATTGGTAAGATCAAGGAAATCAAGGAAGGTTAT
CTGTCCTTGGTAATCCACGAAATTTCGAAAATGGTTATCAAATACAATGCTATTATTGCA
ATGGAGGACTTGTCCTACGGCTTTAAAAAAGGACGCTTTAAGGTGGAGCGCCAGGTTTAT
CAAAAGTTTGAAACAATGCTGATTAACAAGCTGAACTATTTGGTCTTTAAAGATATCTCC
ATCACCGAAAATGGTGGGCTTTTGAAAGGCTATCAACTTACATATATCCCTGATAAGCTT
AAGAATGTGGGTCATCAGTGCGGGTGCATTTTTTATGTTCCTGCAGCCTACACGTCCAAA
ATCGATCCTACAACTGGATTTGTTAATATCTTCAAATTTAAGGATCTTACCGTCGACGCG
AAGCGCGAATTTATCAAGAAATTCGATAGTATTCGTTATGATTCCGAAAAAAACCTTTTC
TGTTTCACCTTTGATTATAATAACTTTATCACGCAAAATACTGTCATGAGCAAATCGAGT
TGGTCTGTGTACACTTACGGAGTACGCATCAAGCGTCGTTTTGTTAATGGGCGCTTCAGT
AACGAGTCAGACACGATTGATATCACAAAAGATATGGAGAAAACGCTGGAGATGACAGAC
ATCAATTGGCGCGATGGTCATGACTTACGTCAAGACATTATCGATTATGAAATTGTCCAG
CATATCTTTGAGATCTTTCGTTTGACTGTTCAGATGCGCAACAGCCTGTCAGAATTGGAG
GATCGTGACTATGATCGCCTTATTTCTCCCGTCTTAAATGAGAACAATATCTTCTACGAC
TCAGCCAAGGCTGGAGATGCACTGCCAAAAGACGCCGACGCAAATGGGGCCTACTGTATT
GCATTGAAGGGGTTGTACGAGATCAAACAGATTACAGAAAATTGGAAGGAGGACGGTAAG
TTCTCTCGTGATAAGCTGAAGATTTCTAACAAAGACTGGTTCGATTTCATTCAGAACAAA
CGTTACCTG
SEQ ID NO: 25
ATGAACAACGGTACCAATAACTTTCAGAATTTCATTGGAATCAGCAGCTTACAGAAAACC
CTGCGCAATGCACTTATCCCCACTGAGACAACCCAGCAGTTCATTGTAAAGAACGGGATT
ATTAAAGAAGATGAGCTTCGCGGGGAGAATCGTCAGATCTTAAAGGATATTATGGACGAT
TACTACCGTGGCTTCATTTCGGAGACGCTGTCGTCGATCGACGACATCGACTGGACATCC
TTGTTTGAAAAGATGGAAATCCAACTGAAGAATGGCGATAACAAGGACACGTTAATCAAA
GAGCAGACGGAATACCGTAAAGCTATCCACAAAAAGTTCGCTAATGACGACCGCTTTAAG
AACATGTTCTCAGCAAAACTTATTAGCGATATTTTACCTGAATTTGTCATCCACAATAAC
AATTACTCCGCGAGTGAAAAAGAGGAGAAAACCCAGGTGATTAAGCTGTTTTCCCGTTTT
GCAACCAGTTTCAAGGACTATTTTAAGAATCGTGCTAATTGTTTCTCTGCAGACGACATT
TCCTCGTCGTCCTGCCATCGCATTGTTAATGATAATGCTGAAATCTTTTTTTCAAACGCA
CTTGTGTATCGTCGCATTGTCAAAAGCTTAAGTAATGACGATATCAATAAGATCTCAGGA
GACATGAAGGACTCCCTGAAAGAAATGTCATTGGAAGAAATTTACTCTTATGAAAAGTAT
GGAGAATTTATTACGCAGGAGGGTATCAGCTTCTATAACGACATTTGTGGTAAAGTGAAC
AGCTTTATGAATCTTTATTGTCAAAAGAATAAAGAGAACAAAAATCTGTACAAGCTGCAG
AAATTGCATAAACAAATTCTGTGCATTGCAGATACTTCGTATGAGGTTCCTTACAAATTC
GAGTCGGATGAGGAGGTGTATCAAAGCGTAAACGGATTTTTGGATAACATTAGTAGTAAG
CATATTGTGGAACGCCTTCGCAAGATTGGTGACAACTATAACGGATACAACTTAGACAAG
ATCTATATTGTCTCGAAGTTTTACGAAAGTGTTTCCCAAAAGACTTATCGCGACTGGGAG
ACAATCAACACTGCGCTGGAAATTCACTATAACAATATCTTGCCGGGGAACGGAAAAAGT
AAGGCAGATAAGGTGAAGAAAGCAGTCAAAAATGATCTGCAAAAAAGCATTACTGAAATT
AACGAACTTGTGTCAAATTACAAATTGTGTTCGGATGACAATATTAAAGCGGAAACGTAT
ATCCACGAGATCTCGCACATTCTTAATAATTTCGAGGCGCAGGAATTAAAGTATAATCCT
GAGATCCATTTGGTGGAATCAGAACTTAAAGCTAGTGAACTGAAAAATGTCCTGGACGTT
ATTATGAATGCATTTCACTGGTGTTCTGTCTTTATGACAGAAGAACTTGTCGACAAAGAC
AACAACTTTTATGCGGAATTAGAAGAGATTTACGACGAAATTTATCCCGTTATTTCGTTA
TATAATTTAGTTCGTAATTACGTGACTCAGAAACCCTACAGCACAAAAAAGATTAAATTA
AACTTTGGGATTCCGACTCTTGCTGATGGATGGAGCAAGTCCAAGGAGTACTCTAATAAC
GCCATTATCTTGATGCGTGACAACCTGTACTACCTGGGCATTTTTAACGCTAAAAACAAA
CCCGACAAAAAGATCATTGAAGGGAACACCTCGGAAAATAAGGGGGACTATAAAAAAATG
ATCTACAATCTGTTGCCAGGCCCAAATAAGATGATCCCAAAGGTTTTTTTATCTTCCAAA
ACTGGCGTAGAAACTTACAAGCCGAGCGCATACATCCTTGAAGGATATAAACAAAACAAA
CATATCAAAAGTTCAAAGGACTTCGATATTACGTTCTGCCATGATTTAATCGATTATTTC
AAGAATTGCATCGCGATTCACCCAGAGTGGAAAAACTTTGGGTTTGATTTTTCAGACACC
AGCACTTACGAGGATATTAGTGGATTCTATCGTGAGGTTGAACTGCAGGGCTATAAAATT
GACTGGACCTATATTTCTGAAAAAGATATTGATCTGCTTCAGGAGAAAGGCCAATTGTAC
TTATTTCAAATCTATAACAAGGATTTCTCCAAGAAGTCCACGGGTAATGACAACTTACAC
ACAATGTATCTGAAGAATCTGTTTAGTGAGGAGAACTTGAAGGACATTGTGCTGAAGCTT
AATGGCGAGGCCGAAATCTTTTTTCGTAAGTCCTCCATTAAAAACCCTATTATCCATAAG
AAAGGGAGTATTCTTGTCAACCGCACGTATGAGGCCGAAGAAAAGGACCAATTCGGAAAC
ATCCAAATTGTCCGTAAAAATATTCCTGAGAACATTTACCAGGAGCTTTACAAGTATTTC
AACGACAAGAGTGATAAAGAACTTTCAGATGAGGCGGCGAAACTGAAGAATGTAGTGGGG
CACCACGAAGCTGCCACGAATATTGTAAAGGATTACCGTTACACCTACGACAAGTACTTT
TTGCATATGCCCATCACAATTAATTTTAAGGCCAATAAAACTGGTTTTATCAACGATCGT
ATCTTACAGTACATTGCTAAGGAAAAAGATCTGCACGTTATCGGTATCGATCGCGGGGAA
CGCAATCTGATTTATGTTAGTGTGATTGACACGTGCGGAAATATTGTTGAGCAGAAGAGC
TTTAATATCGTAAATGGATATGACTATCAAATTAAACTGAAGCAACAGGAAGGGGCCCGC
CAGATTGCCCGCAAGGAGTGGAAAGAAATTGGAAAGATCAAGGAGATTAAAGAAGGGTAC
CTTTCCCTTGTTATCCACGAAATCTCGAAAATGGTGATCAAGTACAATGCCATTATTGCT
ATGGAGGATCTGTCATATGGGTTTAAGAAAGGCCGCTTTAAGGTGGAACGTCAGGTTTAC
CAGAAGTTTGAGACCATGCTTATCAATAAGCTGAATTATCTTGTCTTCAAAGACATCTCA
ATCACAGAGAACGGCGGGCTGTTAAAAGGATATCAGCTGACCTATATCCCCGACAAACTG
AAAAATGTCGGGCACCAATGCGGCTGTATTTTCTACGTGCCCGCTGCATACACATCTAAA
ATTGACCCAACGACTGGATTCGTAAATATTTTTAAGTTTAAGGATCTTACGGTAGATGCA
AAGCGCGAATTTATCAAGAAATTTGATAGTATCCGTTACGACAGCGAGAAAAACTTATTT
TGTTTTACGTTCGATTATAACAACTTCATCACGCAAAATACCGTCATGTCAAAATCTTCC
TGGTCAGTCTATACGTATGGCGTCCGTATCAAGCGCCGCTTCGTCAACGGGCGTTTTTCA
AACGAGTCAGATACCATCGATATCACCAAAGATATGGAAAAAACATTGGAGATGACGGAC
ATCAATTGGCGCGATGGTCATGACTTACGCCAGGACATTATTGACTACGAAATCGTACAA
CATATTTTTGAGATTTTCCGTCTGACCGTGCAAATGCGCAACTCATTATCCGAACTTGAG
GATCGTGATTACGACCGCTTGATCAGTCCTGTTCTGAACGAGAATAATATTTTTTACGAC
AGTGCCAAGGCGGGAGACGCACTGCCCAAGGACGCTGACGCTAACGGAGCTTATTGTATT
GCGTTGAAGGGACTTTACGAAATCAAGCAAATCACTGAAAACTGGAAGGAGGATGGTAAA
TTCTCACGCGACAAGTTGAAAATTTCGAACAAGGACTGGTTCGATTTCATCCAAAACAAG
CGTTATTTA
SEQ ID NO: 26
ATGAACAACGGGACTAATAACTTCCAGAACTTCATCGGTATTTCATCATTACAAAAAACG
CTTCGTAACGCCTTGATCCCAACAGAAACGACCCAACAATTTATTGTAAAAAACGGCATC
ATCAAAGAAGACGAACTGCGTGGCGAAAATCGCCAAATTTTGAAGGACATTATGGATGAC
TATTATCGTGGGTTTATCTCGGAGACATTATCCTCCATCGACGACATTGATTGGACGAGT
CTTTTTGAGAAAATGGAGATCCAGCTTAAAAATGGTGATAACAAGGATACATTGATCAAG
GAGCAAACCGAGTACCGCAAGGCCATCCATAAGAAGTTCGCAAATGACGACCGCTTCAAA
AATATGTTTAGTGCCAAATTGATCTCGGATATCCTTCCTGAGTTCGTAATTCACAACAAT
AATTATAGCGCATCCGAAAAGGAGGAAAAGACTCAAGTCATTAAGCTTTTCAGTCGCTTT
GCTACCTCGTTTAAGGACTATTTCAAGAACCGCGCGAACTGCTTCTCAGCGGATGACATT
TCTTCCTCGTCGTGTCACCGCATCGTGAATGATAATGCGGAGATCTTCTTTAGTAATGCC
TTGGTATACCGCCGCATTGTTAAATCCCTGTCTAACGACGATATCAATAAGATCTCAGGA
GATATGAAGGATAGCCTTAAAGAAATGTCTCTGGAAGAAATTTACTCCTATGAAAAGTAC
GGTGAGTTTATCACCCAAGAGGGGATTAGCTTTTATAACGATATCTGCGGGAAGGTGAAT
TCGTTTATGAACCTTTATTGTCAAAAGAATAAGGAGAATAAGAACTTATATAAGCTTCAG
AAACTGCATAAACAAATCTTATGCATTGCCGATACTAGCTATGAAGTTCCGTATAAATTC
GAGAGCGATGAAGAAGTTTATCAGAGCGTCAATGGGTTCTTGGATAACATTTCATCAAAA
CACATCGTGGAACGTCTGCGTAAGATTGGGGATAACTACAACGGATATAATCTTGACAAA
ATTTATATTGTATCTAAATTCTATGAGTCGGTGAGTCAAAAGACCTACCGTGATTGGGAA
ACAATCAATACCGCGTTAGAAATCCACTATAACAACATTCTGCCAGGGAATGGTAAAAGT
AAAGCGGACAAAGTCAAGAAGGCTGTGAAGAACGATCTGCAAAAGAGTATTACAGAGATT
AACGAATTAGTCTCCAATTATAAGTTATGCTCGGACGATAACATTAAGGCGGAGACGTAT
ATTCATGAGATTTCGCATATTCTTAACAACTTCGAGGCACAAGAGCTTAAGTATAACCCA
GAGATTCACCTTGTCGAATCGGAGCTGAAGGCATCGGAATTAAAAAATGTCTTAGATGTA
ATCATGAACGCGTTCCATTGGTGCAGTGTTTTCATGACTGAGGAGTTAGTTGACAAGGAC
AATAACTTCTACGCAGAATTAGAAGAGATCTATGATGAGATTTATCCAGTGATTTCGCTG
TATAATCTGGTACGTAATTACGTCACTCAAAAGCCCTACTCAACAAAAAAAATTAAGCTG
AACTTCGGAATTCCGACTCTGGCCGACGGGTGGTCCAAGTCAAAGGAGTATTCTAATAAT
GCTATCATCCTGATGCGCGATAACTTATACTATTTGGGAATTTTCAATGCCAAAAATAAA
CCAGATAAAAAGATTATCGAAGGTAATACAAGCGAGAATAAGGGTGACTATAAGAAAATG
ATTTACAATCTTCTTCCAGGCCCTAACAAGATGATTCCCAAAGTTTTTTTGTCCAGTAAA
ACAGGGGTCGAAACTTACAAGCCCAGTGCCTATATCCTTGAAGGGTACAAGCAGAATAAG
CACATCAAATCCTCGAAAGACTTTGATATTACATTTTGTCATGACTTAATCGATTATTTT
AAGAACTGTATCGCAATCCATCCAGAATGGAAGAACTTCGGGTTTGATTTCTCTGATACT
TCCACGTATGAGGATATTTCCGGGTTCTACCGCGAAGTAGAGCTTCAGGGCTATAAAATT
GACTGGACATATATTTCAGAAAAAGACATCGATCTGTTACAAGAAAAAGGACAGTTGTAT
CTGTTTCAAATCTATAATAAGGATTTCTCCAAAAAGTCAACTGGAAATGATAACTTACAT
ACAATGTATCTGAAAAATCTTTTTAGTGAAGAGAATTTGAAGGATATCGTGCTGAAGTTA
AATGGCGAAGCAGAGATCTTCTTCCGCAAGTCCTCGATCAAGAATCCTATCATCCACAAG
AAAGGTAGTATTCTGGTTAACCGCACGTACGAGGCCGAGGAAAAAGACCAGTTCGGTAAT
ATCCAGATTGTACGTAAGAATATTCCTGAAAATATTTACCAGGAATTATACAAGTATTTT
AACGACAAATCGGATAAGGAGCTTTCAGATGAGGCCGCAAAGTTGAAGAACGTCGTAGGA
CACCATGAGGCCGCTACGAATATCGTCAAGGACTACCGCTATACGTATGACAAGTACTTC
CTGCACATGCCTATTACTATCAATTTCAAAGCTAATAAAACAGGATTCATCAATGATCGT
ATCCTTCAGTACATTGCCAAAGAAAAAGATCTGCACGTAATCGGAATCGACCGTGGCGAA
CGTAATCTGATTTACGTATCAGTTATCGACACATGTGGTAACATCGTGGAGCAGAAATCT
TTTAACATTGTTAACGGCTATGATTATCAGATTAAGCTTAAACAGCAGGAGGGGGCACGC
CAAATCGCTCGTAAAGAATGGAAGGAGATTGGAAAGATTAAAGAGATTAAAGAGGGGTAC
CTTTCGCTGGTTATTCACGAAATTTCCAAGATGGTGATTAAGTACAATGCAATCATCGCG
ATGGAAGATCTTAGTTACGGATTCAAAAAGGGACGCTTCAAAGTTGAGCGTCAGGTCTAC
CAGAAATTTGAAACGATGCTGATTAACAAATTGAATTACTTGGTATTCAAAGATATCTCA
ATTACTGAAAATGGTGGCTTATTAAAGGGTTACCAGCTTACCTATATCCCGGATAAGCTG
AAGAACGTGGGCCATCAATGCGGCTGCATCTTTTACGTCCCTGCCGCATATACCTCTAAA
ATTGACCCCACCACCGGATTCGTAAATATTTTTAAATTCAAGGACCTGACGGTGGACGCC
AAGCGCGAATTCATCAAAAAATTCGACTCAATCCGCTATGATTCCGAAAAAAATCTTTTC
TGCTTTACGTTCGATTATAATAACTTCATTACCCAAAACACGGTGATGTCAAAATCGTCC
TGGAGCGTGTATACTTATGGAGTGCGTATCAAGCGCCGCTTTGTTAATGGGCGCTTCAGT
AACGAAAGCGATACCATCGACATTACCAAAGACATGGAGAAGACGCTTGAAATGACGGAT
ATCAATTGGCGTGACGGACACGATCTTCGTCAGGATATCATCGACTACGAGATTGTGCAA
CATATCTTTGAGATTTTCCGTTTAACTGTTCAAATGCGTAACTCCTTGTCCGAATTGGAA
GACCGTGATTACGACCGCTTGATTTCACCAGTGCTTAACGAGAATAACATCTTCTACGAC
TCCGCCAAAGCAGGCGATGCCCTGCCAAAGGACGCTGATGCAAATGGTGCATACTGTATC
GCGTTGAAGGGCTTATACGAGATTAAGCAAATCACCGAAAATTGGAAAGAGGATGGAAAG
TTCAGTCGCGATAAGCTGAAGATCTCTAATAAAGATTGGTTTGACTTTATCCAGAACAAA
CGTTATTTA
SEQ ID NO: 27
ATGAACAACGGTACCAATAATTTCCAAAATTTCATCGGAATCTCATCCTTGCAAAAAACC
TTGCGCAATGCTTTGATCCCCACCGAAACCACGCAGCAGTTCATCGTGAAAAACGGCATT
ATCAAAGAGGATGAGTTGCGCGGGGAAAACCGTCAAATTCTTAAGGATATCATGGACGAT
TACTACCGTGGGTTTATCAGTGAGACCCTGTCAAGCATTGACGACATTGACTGGACCAGC
TTATTTGAGAAGATGGAGATTCAATTAAAGAACGGGGACAATAAGGACACGCTTATCAAA
GAGCAGACAGAATACCGTAAAGCGATTCATAAGAAATTTGCAAATGACGATCGCTTCAAG
AACATGTTTTCAGCAAAATTAATCAGCGACATCCTTCCCGAATTTGTGATTCATAATAAC
AACTATTCGGCTAGCGAAAAAGAGGAGAAAACTCAGGTTATTAAGCTTTTCTCGCGTTTT
GCCACTTCGTTCAAAGACTATTTTAAGAATCGCGCAAACTGCTTTTCGGCTGATGATATT
TCCAGTTCTAGCTGCCATCGTATCGTTAACGATAATGCTGAGATTTTCTTCTCTAATGCC
CTGGTGTATCGTCGTATCGTTAAATCTTTGAGCAACGACGATATTAATAAGATTTCAGGC
GACATGAAGGATTCTTTAAAGGAGATGTCTTTAGAAGAGATTTATTCCTATGAGAAATAT
GGCGAGTTTATCACCCAAGAAGGAATTTCGTTCTACAACGACATCTGTGGCAAAGTGAAC
AGCTTCATGAATTTATACTGCCAAAAGAATAAGGAGAATAAAAATTTATATAAACTGCAG
AAACTGCATAAGCAAATTCTTTGCATTGCAGACACCTCTTATGAAGTTCCTTATAAGTTT
GAATCGGACGAGGAGGTATATCAGAGTGTGAACGGGTTCCTGGACAATATTTCATCCAAG
CATATTGTTGAACGTTTACGCAAAATTGGAGACAATTACAATGGGTATAACCTTGACAAA
ATTTACATCGTGTCGAAGTTTTACGAATCGGTAAGCCAGAAGACCTATCGTGACTGGGAA
ACTATCAATACCGCCTTAGAAATTCATTACAACAATATTCTTCCTGGTAACGGCAAAAGC
AAAGCCGATAAGGTAAAGAAGGCTGTCAAGAACGACCTGCAAAAGTCTATCACAGAGATC
AACGAGTTAGTCTCTAACTACAAATTATGTTCCGACGACAATATTAAAGCCGAAACCTAC
ATCCATGAGATCTCACACATTCTTAACAATTTTGAGGCCCAGGAGCTGAAATATAACCCA
GAAATTCACCTTGTAGAGAGCGAATTAAAAGCCTCCGAGCTGAAGAACGTTTTGGATGTA
ATCATGAACGCATTTCATTGGTGCAGCGTATTTATGACAGAGGAGTTGGTCGACAAGGAC
AATAACTTTTACGCCGAGCTTGAAGAAATCTACGATGAAATTTACCCGGTAATTAGTTTA
TATAATTTAGTTCGCAACTACGTAACTCAGAAACCCTACAGTACCAAGAAGATTAAATTG
AACTTTGGGATCCCGACACTTGCTGACGGTTGGAGTAAATCAAAAGAATACTCCAATAAT
GCAATTATCCTGATGCGCGACAATCTTTACTACTTGGGGATCTTTAACGCAAAGAACAAA
CCAGATAAGAAAATCATCGAGGGCAACACCAGCGAGAATAAAGGCGATTACAAGAAAATG
ATCTATAATCTTTTGCCGGGACCGAACAAAATGATCCCAAAGGTTTTCCTGTCGTCGAAA
ACGGGAGTCGAGACATATAAACCATCTGCGTACATCTTGGAAGGTTACAAACAGAATAAG
CATATTAAGTCTAGTAAAGACTTCGACATCACCTTTTGTCATGACCTGATTGATTATTTC
AAGAACTGTATTGCTATCCATCCAGAATGGAAAAACTTCGGATTTGACTTCTCCGATACT
AGCACCTACGAAGACATTTCGGGTTTTTATCGCGAAGTAGAGCTTCAAGGGTACAAAATT
GATTGGACATATATTAGCGAGAAAGACATTGATTTGCTTCAAGAGAAGGGACAGTTATAT
TTATTCCAGATCTACAACAAAGACTTCTCGAAGAAATCCACCGGTAATGATAATCTTCAC
ACTATGTACCTGAAGAATTTATTTTCAGAGGAAAATCTGAAGGACATTGTACTTAAACTT
AATGGAGAAGCCGAAATCTTCTTCCGCAAGAGTTCCATTAAAAATCCGATTATTCATAAA
AAGGGAAGTATCCTTGTGAACCGCACGTATGAGGCCGAAGAGAAGGATCAGTTTGGGAAT
ATTCAAATTGTCCGCAAAAACATCCCCGAGAACATCTACCAGGAACTGTATAAATACTTT
AATGATAAATCTGATAAAGAGTTATCAGACGAGGCTGCCAAACTGAAAAACGTAGTCGGT
CATCATGAGGCAGCGACCAATATTGTAAAGGACTACCGTTACACCTACGACAAGTATTTC
CTTCACATGCCGATCACGATTAATTTTAAGGCTAACAAGACCGGCTTTATCAATGACCGC
ATCTTGCAGTACATCGCGAAAGAGAAAGATTTACACGTCATCGGAATTGATCGTGGAGAG
CGTAATCTTATCTACGTCAGCGTCATCGACACCTGTGGAAACATTGTGGAACAAAAAAGT
TTTAATATCGTAAACGGCTACGACTATCAAATTAAACTTAAACAGCAAGAGGGAGCTCGC
CAGATCGCTCGCAAAGAGTGGAAAGAGATTGGGAAAATTAAAGAAATTAAAGAGGGTTAC
CTGTCGCTGGTAATTCACGAAATCTCGAAAATGGTCATCAAATATAATGCAATTATCGCT
ATGGAGGATCTGTCCTACGGGTTCAAGAAGGGACGTTTTAAAGTAGAGCGCCAGGTGTAT
CAAAAATTCGAAACCATGTTGATCAATAAGCTTAACTATTTGGTCTTCAAAGATATTTCG
ATTACGGAGAACGGAGGTTTGTTGAAAGGATATCAGCTGACGTATATCCCAGACAAGTTG
AAAAACGTGGGGCATCAATGTGGATGTATTTTCTATGTGCCCGCGGCCTACACGAGTAAG
ATCGATCCTACCACTGGTTTCGTCAACATTTTCAAATTTAAAGATCTTACCGTGGATGCG
AAGCGCGAATTTATTAAGAAATTTGATAGCATTCGCTATGATTCCGAAAAGAACCTGTTC
TGTTTTACGTTCGACTATAACAATTTCATTACCCAAAACACGGTGATGAGCAAATCCTCT
TGGTCAGTTTATACATACGGTGTACGTATCAAACGCCGTTTCGTTAACGGACGCTTTTCC
AATGAGTCTGATACAATCGATATCACGAAAGATATGGAAAAAACATTAGAGATGACTGAT
ATCAACTGGCGTGACGGGCACGACCTGCGTCAAGACATTATTGACTACGAGATTGTGCAG
CATATCTTCGAAATCTTTCGCTTAACTGTGCAAATGCGTAACTCGTTATCCGAGTTAGAA
GACCGTGACTACGATCGCCTGATTTCACCCGTCTTGAACGAAAATAACATCTTCTACGAT
TCCGCGAAGGCTGGGGACGCATTGCCCAAGGACGCAGACGCGAATGGAGCGTACTGTATT
GCGCTTAAAGGATTATATGAAATCAAGCAGATCACCGAAAATTGGAAGGAGGACGGGAAG
TTCTCACGCGACAAACTGAAGATTTCAAATAAGGACTGGTTCGATTTCATTCAGAATAAG
CGTTACCTG
SEQ ID NO: 28
TGAATAATGGTACGAACAACTTTCAGAACTTCATCGGCATCTCCAGCCTTCAAAAGACTT
TACGCAACGCATTGATTCCCACGGAGACTACGCAACAGTTTATCGTAAAAAATGGTATTA
TCAAAGAAGATGAATTACGCGGGGAGAATCGCCAGATTCTTAAGGACATTATGGACGATT
ATTACCGTGGATTCATCAGTGAGACACTGAGCTCCATTGATGACATCGACTGGACGTCAT
TGTTTGAAAAGATGGAAATCCAGTTGAAAAATGGCGATAACAAAGATACATTGATTAAAG
AGCAGACAGAGTACCGCAAAGCAATTCACAAGAAATTCGCCAATGATGATCGTTTTAAGA
ACATGTTTAGTGCCAAGCTTATTTCGGATATCTTACCCGAATTCGTGATTCACAACAACA
ATTATTCGGCAAGTGAGAAAGAGGAAAAGACCCAGGTTATCAAATTGTTTTCGCGCTTCG
CCACTTCGTTCAAAGATTATTTCAAGAACCGTGCAAACTGTTTCTCCGCTGACGACATCA
GTTCCAGCTCATGCCACCGTATTGTAAATGACAATGCGGAGATCTTTTTCAGTAATGCCT
TAGTATATCGTCGCATTGTAAAGAGCTTATCTAATGATGACATTAACAAGATCTCGGGTG
ATATGAAGGACTCACTTAAGGAGATGAGTCTGGAAGAGATCTACTCCTACGAAAAATACG
GGGAATTCATCACCCAGGAGGGAATTTCATTCTACAACGATATCTGCGGCAAAGTTAACT
CCTTTATGAATCTGTACTGTCAAAAGAACAAGGAGAATAAAAACCTGTATAAATTGCAGA
AACTTCATAAACAAATTTTGTGTATCGCAGACACGAGTTATGAAGTACCTTATAAATTCG
AATCCGACGAAGAGGTATATCAGTCCGTAAATGGGTTCCTGGACAATATCAGTAGTAAGC
ACATTGTGGAACGCTTACGCAAAATTGGAGACAATTACAACGGGTATAACCTGGACAAAA
TCTACATCGTATCCAAATTTTATGAAAGCGTGTCTCAAAAAACTTATCGTGATTGGGAAA
CAATCAACACGGCTCTTGAGATCCATTACAATAACATCTTGCCGGGTAACGGCAAATCGA
AGGCAGACAAAGTTAAAAAAGCAGTTAAGAACGACTTACAGAAAAGCATTACGGAGATTA
ACGAGTTAGTAAGTAATTACAAATTATGCTCCGACGATAATATCAAAGCTGAAACCTACA
TCCATGAAATTAGCCACATTTTGAACAATTTCGAAGCGCAGGAGCTGAAATATAACCCTG
AAATCCATCTGGTAGAGTCTGAGTTGAAGGCGTCAGAACTGAAAAACGTTCTTGACGTCA
TCATGAATGCCTTTCACTGGTGTAGTGTTTTTATGACTGAGGAGCTTGTAGATAAGGACA
ACAACTTCTATGCTGAACTTGAAGAGATCTACGATGAAATCTACCCCGTAATCAGTCTGT
ATAATTTAGTTCGTAACTACGTCACGCAGAAACCCTATTCGACTAAGAAAATTAAGCTGA
ACTTTGGGATCCCTACTTTGGCAGACGGGTGGAGCAAGAGTAAAGAATACAGTAATAATG
CAATTATCTTGATGCGCGATAACTTATATTACTTAGGTATTTTCAATGCTAAGAACAAAC
CTGATAAGAAGATTATCGAAGGAAATACGAGTGAGAATAAGGGAGACTACAAAAAGATGA
TTTACAACTTGCTGCCAGGGCCTAATAAGATGATTCCAAAAGTTTTTCTGTCGAGCAAGA
CAGGGGTTGAAACTTATAAGCCATCCGCTTATATCCTTGAGGGGTACAAGCAGAATAAGC
ATATCAAGTCCTCCAAAGATTTTGATATTACATTTTGCCACGACTTAATTGATTACTTCA
AGAACTGCATCGCAATCCATCCCGAATGGAAGAATTTCGGCTTCGATTTCTCAGATACGT
CCACGTATGAGGATATCTCAGGCTTTTACCGCGAAGTTGAGCTGCAAGGTTATAAAATTG
ATTGGACATACATCTCCGAAAAAGACATTGATCTTTTACAGGAAAAGGGCCAATTATACT
TATTTCAAATCTATAACAAAGATTTTAGCAAGAAGTCCACAGGTAATGATAACCTGCATA
CGATGTATTTGAAAAATCTTTTCAGTGAAGAGAATTTGAAGGATATCGTCCTGAAGCTGA
ACGGTGAGGCTGAGATCTTCTTCCGCAAATCGTCTATCAAAAACCCCATCATTCACAAAA
AGGGAAGTATCTTAGTAAACCGCACTTATGAAGCGGAGGAAAAGGATCAGTTCGGGAACA
TCCAGATCGTGCGCAAGAACATTCCAGAAAACATCTATCAGGAACTTTACAAATATTTCA
ATGACAAGTCTGATAAAGAATTATCAGACGAGGCGGCGAAACTTAAAAATGTTGTTGGAC
ACCACGAAGCAGCGACGAATATTGTAAAGGATTATCGCTACACATACGATAAATACTTTT
TGCACATGCCAATCACCATTAACTTTAAGGCGAACAAGACAGGTTTCATTAACGACCGTA
TTCTGCAATATATCGCAAAGGAAAAAGACCTGCACGTTATTGGGATCGATCGTGGCGAAC
GCAATTTGATCTACGTAAGCGTTATCGACACTTGCGGAAATATCGTTGAACAAAAAAGCT
TTAATATCGTCAATGGATACGATTACCAAATCAAGCTGAAACAACAAGAAGGGGCACGTC
AGATCGCTCGTAAAGAATGGAAAGAGATTGGTAAGATCAAAGAGATTAAAGAAGGGTATC
TTTCTTTAGTAATTCACGAGATTTCGAAAATGGTTATTAAATACAATGCGATTATTGCTA
TGGAAGACTTAAGCTACGGCTTTAAGAAAGGTCGCTTCAAAGTGGAGCGCCAAGTGTATC
AGAAGTTTGAAACGATGTTGATTAACAAATTAAATTACCTGGTCTTTAAGGACATCAGTA
TCACAGAAAATGGGGGGTTGCTTAAAGGGTACCAGCTTACATACATCCCTGATAAACTGA
AAAATGTCGGTCATCAGTGCGGATGTATCTTCTATGTACCAGCAGCCTATACCAGTAAGA
TTGACCCTACTACTGGCTTTGTGAATATTTTTAAATTCAAGGATTTAACCGTGGACGCCA
AGCGTGAATTTATTAAAAAATTTGATTCGATTCGCTACGACAGTGAGAAAAACCTTTTCT
GCTTTACCTTTGACTACAACAATTTTATTACCCAGAACACCGTAATGTCAAAGAGTTCGT
GGTCTGTATATACCTACGGTGTTCGCATCAAGCGCCGCTTCGTAAACGGGCGTTTCAGTA
ACGAATCTGACACCATCGACATCACTAAAGATATGGAGAAGACATTGGAAATGACGGACA
TTAATTGGCGTGATGGCCATGACTTACGTCAGGACATTATTGATTACGAAATTGTGCAGC
ATATCTTCGAGATTTTCCGTTTGACAGTTCAGATGCGCAACTCACTGAGTGAGTTAGAAG
ATCGCGATTACGACCGTCTGATCTCACCGGTCCTTAATGAAAACAACATTTTCTACGACT
CAGCAAAGGCGGGTGATGCCCTGCCAAAGGATGCGGACGCTAATGGCGCCTACTGCATCG
CCCTGAAAGGATTGTATGAAATTAAGCAGATTACAGAAAATTGGAAGGAAGATGGTAAAT
TTAGCCGTGATAAATTAAAAATCTCGAACAAGGATTGGTTCGATTTTATTCAGAACAAAC
GTTATTTG
SEQ ID NO: 29
ATGAACAATGGAACAAATAATTTTCAAAATTTTATCGGCATCTCAAGTCTTCAAAAAACC
CTTCGCAATGCCCTGATTCCAACTGAAACAACCCAGCAATTTATCGTCAAGAACGGCATC
ATTAAGGAAGACGAGTTACGCGGGGAGAACCGTCAAATCCTGAAAGATATCATGGATGAC
TACTATCGTGGGTTCATTTCGGAAACCTTGTCTTCAATCGACGACATTGACTGGACGAGT
CTTTTCGAGAAAATGGAAATTCAGCTTAAAAATGGAGACAACAAGGATACTCTGATTAAG
GAACAGACAGAATATCGCAAAGCTATCCACAAAAAGTTCGCTAATGATGATCGTTTCAAA
AATATGTTTTCTGCTAAATTGATTTCCGATATCTTGCCTGAATTTGTAATCCACAACAAC
AATTATTCTGCTTCCGAGAAGGAAGAGAAGACCCAGGTCATTAAATTATTCAGCCGCTTT
GCAACCAGCTTTAAAGACTACTTTAAGAATCGCGCTAACTGCTTTTCGGCGGATGACATC
TCATCATCATCATGCCACCGCATTGTGAACGACAATGCGGAGATCTTCTTTTCGAATGCG
TTAGTTTATCGTCGCATTGTCAAAAGTCTTAGCAATGATGACATCAACAAGATCTCAGGA
GACATGAAAGATTCCTTAAAGGAGATGTCTCTTGAGGAAATCTATTCGTATGAGAAATAC
GGCGAGTTCATTACCCAGGAAGGTATTAGTTTCTACAATGATATCTGCGGCAAAGTAAAT
TCTTTTATGAATCTGTATTGCCAAAAAAACAAAGAAAACAAGAATCTTTATAAGTTACAA
AAGTTACATAAGCAAATTCTGTGCATCGCTGATACATCTTATGAGGTACCCTACAAATTT
GAAAGTGATGAGGAGGTCTATCAGAGTGTCAACGGCTTCTTAGACAACATCTCTTCCAAA
CATATCGTGGAACGCCTGCGTAAAATCGGAGATAACTACAACGGATATAACTTAGATAAA
ATCTACATCGTGTCCAAGTTTTATGAAAGTGTGAGCCAAAAAACATATCGTGACTGGGAA
ACCATTAACACCGCATTGGAAATTCACTATAACAACATTTTGCCAGGCAACGGGAAAAGT
AAGGCGGACAAAGTTAAGAAAGCAGTTAAAAATGACCTGCAAAAAAGCATCACTGAAATT
AACGAATTGGTATCGAATTACAAATTATGTAGCGACGATAATATCAAAGCAGAAACTTAC
ATTCACGAGATTAGTCACATTTTAAATAACTTCGAGGCCCAGGAATTGAAATACAATCCC
GAAATTCATTTGGTTGAATCAGAACTGAAAGCATCAGAGTTGAAAAATGTGTTAGATGTC
ATTATGAATGCGTTTCATTGGTGCTCTGTGTTCATGACCGAGGAACTGGTTGATAAAGAT
AACAACTTTTACGCTGAATTGGAGGAGATTTACGATGAGATTTACCCGGTCATTTCGCTT
TATAACTTAGTGCGCAATTATGTGACGCAGAAACCATATTCCACGAAGAAAATCAAACTT
AATTTTGGCATCCCTACTCTGGCTGATGGTTGGTCGAAATCGAAAGAGTACAGCAACAAC
GCGATCATTCTTATGCGTGACAATCTTTACTATTTGGGCATTTTTAATGCCAAGAATAAG
CCAGATAAGAAAATCATTGAGGGGAATACTTCCGAGAATAAGGGGGATTACAAAAAGATG
ATCTATAACTTGCTGCCCGGCCCCAACAAAATGATTCCTAAGGTTTTCTTGTCAAGCAAG
ACGGGCGTCGAAACATATAAGCCGTCAGCTTATATTCTGGAAGGCTATAAACAGAATAAG
CACATCAAGTCTTCCAAGGACTTTGACATCACTTTTTGCCACGATTTGATCGACTACTTT
AAGAACTGTATTGCGATTCATCCGGAATGGAAGAACTTCGGTTTCGACTTTTCCGATACC
TCAACATACGAGGATATCAGCGGCTTCTACCGTGAAGTCGAGCTTCAAGGCTACAAGATC
GATTGGACATATATTTCAGAGAAGGACATTGATTTGTTACAAGAGAAAGGTCAACTTTAC
TTATTTCAGATCTATAACAAAGACTTTTCGAAGAAATCGACAGGAAACGATAACTTACAC
ACTATGTATTTAAAAAATCTGTTTTCGGAGGAAAACCTGAAAGATATTGTGCTGAAACTT
AACGGCGAGGCAGAGATCTTTTTCCGTAAAAGCTCAATCAAGAATCCTATCATCCATAAA
AAAGGTAGTATTCTTGTCAACCGCACATATGAAGCGGAGGAGAAGGACCAATTCGGAAAC
ATCCAAATTGTCCGTAAGAATATTCCGGAGAACATTTACCAAGAGTTGTATAAATACTTT
AACGATAAGTCAGATAAGGAACTTAGCGATGAGGCGGCGAAGCTTAAAAACGTAGTTGGG
CATCATGAAGCTGCTACCAACATTGTAAAAGATTACCGTTACACCTATGACAAGTATTTC
TTGCACATGCCCATTACGATCAATTTCAAAGCAAATAAGACAGGCTTTATCAATGATCGC
ATCCTGCAGTACATTGCTAAAGAGAAGGATTTGCATGTTATCGGTATTGATCGCGGAGAG
CGCAATTTGATCTACGTCTCCGTAATCGACACTTGCGGTAACATTGTTGAGCAGAAGTCG
TTCAACATCGTTAATGGTTATGATTACCAAATCAAGCTGAAGCAGCAAGAGGGTGCCCGC
CAGATCGCGCGTAAGGAATGGAAAGAAATCGGGAAAATTAAAGAGATCAAAGAAGGCTAT
TTGTCTCTGGTAATTCACGAAATCAGCAAGATGGTGATCAAGTATAACGCGATCATTGCG
ATGGAGGATCTTTCTTATGGCTTCAAGAAAGGGCGCTTTAAAGTCGAACGCCAGGTCTAC
CAGAAATTTGAGACAATGCTTATCAACAAGCTTAACTATCTTGTATTTAAGGATATTTCC
ATCACTGAGAACGGAGGACTTTTAAAGGGGTACCAACTGACGTACATTCCTGATAAGCTG
AAGAACGTTGGTCATCAATGCGGATGCATCTTCTATGTGCCAGCGGCTTACACCTCCAAA
ATCGATCCCACTACAGGCTTTGTCAATATCTTCAAATTCAAGGATTTGACCGTTGACGCG
AAGCGCGAGTTTATCAAGAAGTTTGATAGCATTCGCTACGACAGCGAAAAAAATTTATTT
TGTTTTACTTTCGACTACAATAACTTTATTACTCAGAACACTGTCATGTCAAAGAGTTCG
TGGAGTGTCTACACGTACGGAGTACGTATTAAGCGCCGTTTCGTCAACGGACGCTTCTCA
AACGAAAGCGACACGATCGACATCACCAAAGACATGGAAAAAACTCTTGAGATGACGGAT
ATCAATTGGCGCGACGGCCATGACCTGCGTCAGGATATCATTGATTACGAGATCGTTCAG
CACATCTTCGAAATCTTCCGCCTTACCGTCCAGATGCGCAACAGTTTAAGCGAGCTTGAA
GACCGCGACTACGATCGTTTGATTAGCCCCGTTCTGAACGAGAATAATATTTTCTACGAC
AGCGCAAAGGCCGGTGATGCTTTGCCAAAGGACGCAGACGCGAATGGAGCCTACTGCATC
GCCCTGAAGGGCTTATATGAGATTAAGCAAATTACCGAAAATTGGAAGGAAGATGGTAAG
TTCTCCCGTGATAAGCTTAAAATTAGCAATAAGGATTGGTTCGACTTCATCCAGAACAAA
CGTTACCTG
SEQ ID NO: 30
ATGAACAACGGAACAAACAATTTCCAAAACTTCATCGGTATCTCTTCGTTGCAGAAGACT
CTGCGTAATGCTTTGATCCCGACGGAGACAACCCAACAATTTATCGTCAAAAACGGTATT
ATTAAGGAGGACGAGTTACGTGGAGAAAATCGTCAAATCCTTAAGGACATCATGGACGAT
TATTATCGCGGGTTTATTTCTGAAACCCTGAGCAGTATCGATGATATCGACTGGACCTCA
CTTTTTGAGAAAATGGAGATCCAGTTGAAGAACGGTGATAACAAAGACACTCTGATCAAA
GAGCAAACTGAATACCGCAAGGCAATTCACAAAAAGTTCGCCAACGACGACCGTTTCAAG
AATATGTTCTCAGCTAAGTTAATCAGCGACATTTTGCCAGAGTTCGTTATCCACAACAAT
AATTATAGTGCTTCAGAGAAGGAGGAAAAAACCCAAGTGATTAAACTTTTTTCGCGCTTT
GCAACCTCATTCAAGGACTACTTCAAGAATCGCGCGAATTGCTTCAGTGCGGACGACATT
TCTTCTTCAAGTTGCCATCGTATCGTTAACGATAACGCGGAAATTTTCTTCTCTAATGCT
TTGGTGTATCGCCGCATTGTAAAATCGCTTAGTAACGATGACATTAATAAGATCTCAGGT
GATATGAAAGATTCATTGAAGGAAATGAGCTTGGAAGAGATTTACAGTTACGAAAAATAT
GGAGAATTTATTACTCAGGAAGGCATCTCATTCTATAACGATATCTGCGGGAAGGTAAAT
TCGTTTATGAACTTATATTGCCAGAAAAATAAAGAGAATAAAAATTTGTATAAGCTTCAG
AAGTTGCACAAACAGATCCTGTGCATTGCAGACACCTCGTATGAGGTTCCGTATAAATTT
GAGTCCGATGAAGAAGTGTATCAGTCTGTGAATGGTTTCTTAGATAATATCTCTTCCAAG
CATATTGTCGAACGCCTGCGCAAAATTGGTGATAACTATAACGGATACAATCTGGATAAA
ATTTACATCGTTTCTAAATTTTACGAGTCAGTCTCGCAGAAGACCTACCGCGACTGGGAA
ACAATTAACACGGCATTGGAGATTCACTACAATAATATCTTGCCTGGTAACGGTAAGTCT
AAGGCAGATAAGGTAAAAAAAGCTGTGAAAAACGACCTTCAGAAAAGCATCACGGAGATT
AATGAGCTGGTGAGTAATTACAAATTATGTTCAGACGATAATATTAAAGCTGAAACGTAT
ATCCATGAAATCTCGCATATCTTGAACAACTTCGAGGCCCAAGAACTTAAATATAACCCC
GAAATCCATTTAGTCGAGTCTGAATTGAAAGCGTCGGAATTAAAAAACGTCTTAGACGTC
ATTATGAACGCGTTTCACTGGTGTTCAGTTTTCATGACCGAAGAGCTGGTCGACAAAGAC
AACAACTTCTATGCGGAATTGGAGGAAATCTATGATGAAATCTACCCTGTTATTTCACTG
TATAACCTTGTGCGCAACTATGTCACTCAGAAGCCGTATTCGACCAAAAAAATTAAATTG
AATTTCGGTATCCCTACTCTTGCAGACGGATGGAGTAAAAGCAAGGAATACAGTAATAAC
GCCATTATTCTTATGCGCGACAATTTATACTACCTGGGCATCTTTAACGCAAAGAATAAG
CCGGATAAGAAGATTATTGAGGGTAACACCAGTGAGAACAAGGGCGACTATAAGAAGATG
ATCTATAACTTATTGCCAGGTCCAAATAAAATGATCCCAAAAGTATTCTTATCATCAAAG
ACGGGAGTTGAAACCTATAAGCCTAGTGCCTATATTCTTGAGGGATATAAACAGAACAAG
CACATTAAGTCGTCTAAGGATTTTGACATTACGTTCTGCCATGACTTAATCGACTATTTT
AAAAACTGTATTGCGATTCACCCCGAATGGAAGAATTTTGGATTCGATTTTTCGGATACC
TCGACCTATGAAGATATTTCGGGATTTTATCGTGAAGTGGAGTTGCAAGGCTATAAAATC
GATTGGACCTATATCTCAGAAAAAGACATTGATTTATTACAGGAAAAGGGACAACTGTAC
CTTTTCCAAATTTATAACAAGGACTTTTCTAAAAAGTCCACAGGAAATGATAACCTTCAC
ACCATGTACCTGAAGAACCTTTTCTCAGAGGAAAACCTGAAGGACATTGTCCTTAAGTTA
AATGGAGAAGCGGAGATCTTTTTCCGTAAATCTAGTATCAAGAATCCGATTATCCATAAA
AAAGGTTCGATTTTGGTAAATCGCACCTATGAAGCGGAAGAGAAAGATCAATTTGGTAAC
ATCCAGATCGTGCGCAAGAATATCCCGGAGAACATTTACCAAGAGCTGTATAAGTACTTC
AATGATAAGTCTGATAAGGAACTGTCAGATGAAGCTGCGAAATTGAAGAACGTGGTTGGG
CATCATGAAGCCGCTACCAATATCGTCAAGGATTACCGTTATACCTATGACAAATATTTC
TTACACATGCCGATTACGATCAATTTTAAGGCAAACAAGACAGGATTCATCAACGACCGT
ATCTTGCAGTATATTGCCAAAGAGAAGGATCTGCATGTGATCGGTATTGACCGCGGGGAG
CGCAATTTAATCTATGTATCGGTGATCGATACTTGTGGTAACATCGTAGAACAAAAGAGC
TTTAACATCGTGAATGGTTACGACTATCAGATCAAGCTGAAACAACAGGAAGGAGCCCGC
CAGATCGCTCGCAAGGAATGGAAAGAAATCGGGAAAATTAAGGAAATCAAGGAAGGCTAC
CTTTCATTGGTCATTCACGAAATTTCGAAAATGGTAATTAAGTACAACGCGATCATCGCC
ATGGAGGACCTTTCGTACGGATTTAAGAAGGGTCGTTTCAAAGTTGAGCGCCAGGTATAC
CAAAAATTCGAGACTATGCTTATCAACAAACTTAACTACTTGGTCTTTAAGGACATTTCT
ATTACCGAAAACGGCGGCTTACTTAAAGGCTATCAATTGACATATATTCCCGACAAACTG
AAGAATGTTGGACATCAATGCGGGTGTATTTTCTATGTGCCGGCAGCTTACACTAGTAAG
ATCGACCCTACAACCGGGTTCGTAAACATTTTTAAATTCAAAGACTTAACAGTCGATGCG
AAGCGTGAATTTATTAAGAAGTTTGATAGTATCCGCTATGACAGTGAAAAGAACTTGTTT
TGCTTTACGTTCGACTACAATAACTTTATTACACAGAACACGGTCATGTCTAAATCATCA
TGGTCGGTTTACACATATGGGGTGCGCATCAAGCGTCGCTTTGTAAATGGCCGTTTTAGT
AATGAGAGCGACACAATCGACATCACAAAGGATATGGAGAAAACTCTTGAGATGACAGAC
ATCAATTGGCGTGACGGTCATGACTTACGCCAAGATATCATCGACTACGAAATCGTACAG
CATATTTTTGAGATTTTTCGTCTTACTGTGCAAATGCGTAATTCTTTATCCGAACTGGAA
GATCGTGATTACGACCGCTTGATTAGTCCCGTCTTAAATGAGAACAATATTTTCTATGAT
TCTGCGAAAGCCGGAGATGCACTGCCCAAAGACGCTGATGCCAATGGCGCGTATTGCATT
GCATTAAAAGGATTATATGAGATTAAACAGATTACCGAAAATTGGAAAGAGGACGGTAAA
TTCTCACGCGATAAATTGAAGATTTCTAACAAGGACTGGTTCGACTTTATCCAAAATAAA
CGTTATCTT
SEQ ID NO: 31
ATGAATAACGGTACCAACAACTTTCAGAATTTCATTGGCATTAGCTCGCTTCAAAAAACT
TTACGCAATGCTCTTATTCCGACTGAGACGACACAACAGTTTATCGTTAAGAATGGCATC
ATCAAAGAAGATGAATTACGCGGAGAAAACCGCCAGATCCTGAAAGACATTATGGACGAT
TATTACCGTGGGTTCATCTCCGAGACGTTGTCATCGATCGATGACATCGACTGGACGTCA
CTTTTTGAAAAAATGGAGATCCAGTTAAAGAACGGTGACAATAAGGATACATTGATCAAA
GAACAGACCGAGTACCGTAAAGCGATTCATAAAAAGTTTGCGAACGATGATCGCTTCAAG
AATATGTTTTCTGCGAAATTAATTTCCGACATTTTACCTGAATTTGTTATTCATAATAAC
AACTACTCGGCGTCTGAGAAAGAGGAGAAAACCCAAGTGATTAAACTTTTTTCACGTTTC
GCAACGTCGTTCAAAGACTATTTTAAAAATCGTGCTAATTGCTTTAGCGCGGATGACATC
AGCTCTAGTTCATGTCATCGCATTGTCAACGATAATGCTGAGATCTTTTTCAGTAATGCG
TTAGTGTACCGTCGTATTGTGAAGTCCTTATCTAATGATGATATCAATAAGATCAGCGGG
GATATGAAGGACTCACTTAAGGAGATGAGCTTGGAGGAAATCTATTCCTATGAGAAGTAT
GGTGAGTTTATTACGCAAGAAGGAATTAGCTTTTACAACGATATCTGTGGAAAGGTGAAT
TCGTTTATGAATTTGTATTGCCAGAAAAATAAGGAGAACAAGAACCTTTATAAATTGCAA
AAGTTACACAAGCAAATCCTGTGCATTGCAGATACTTCCTACGAGGTGCCTTACAAGTTT
GAATCCGACGAAGAGGTCTACCAATCTGTAAACGGTTTCTTAGATAATATTAGTTCCAAG
CATATTGTGGAGCGCCTTCGTAAAATTGGCGATAATTACAACGGTTACAATTTAGACAAA
ATTTACATTGTCAGTAAATTCTACGAGTCCGTATCTCAAAAGACGTATCGTGATTGGGAG
ACTATCAATACGGCCCTGGAGATCCACTACAACAATATCTTGCCCGGTAATGGTAAGTCG
AAGGCCGATAAAGTTAAGAAAGCGGTGAAAAATGACTTACAGAAGTCAATCACCGAAATT
AACGAATTGGTGTCCAATTATAAATTGTGTTCAGATGATAATATCAAAGCCGAGACCTAC
ATTCATGAGATTTCCCATATCTTAAATAATTTCGAGGCGCAAGAGCTTAAGTATAACCCA
GAAATCCACCTGGTAGAATCTGAGTTGAAGGCGTCAGAGTTAAAAAATGTTTTAGATGTC
ATTATGAACGCGTTTCACTGGTGCTCCGTATTTATGACGGAGGAATTAGTAGATAAAGAC
AACAATTTCTATGCCGAACTTGAGGAAATCTATGATGAGATCTATCCCGTCATTAGCCTG
TATAACTTGGTCCGCAACTATGTTACCCAAAAACCGTACAGTACCAAGAAGATTAAGCTG
AATTTCGGCATTCCTACACTGGCTGATGGTTGGAGTAAATCGAAGGAATATTCGAATAAC
GCGATTATCTTGATGCGCGACAACTTATACTATTTGGGGATCTTTAACGCCAAAAACAAA
CCGGATAAGAAGATTATTGAGGGAAACACATCAGAGAACAAAGGCGACTACAAAAAAATG
ATTTACAACTTGTTACCGGGGCCTAACAAAATGATCCCGAAGGTGTTCTTATCCAGTAAA
ACAGGCGTTGAGACCTACAAACCTTCCGCATACATCCTGGAAGGGTATAAGCAGAACAAG
CACATTAAGTCCAGCAAGGATTTCGATATTACCTTCTGTCATGATTTAATTGACTATTTC
AAGAACTGTATTGCAATCCACCCCGAGTGGAAGAACTTCGGATTCGACTTCTCAGATACG
AGCACATATGAGGACATCTCGGGGTTCTATCGTGAAGTAGAACTGCAGGGATATAAAATT
GATTGGACATATATTTCCGAAAAAGACATCGACCTTTTACAAGAGAAGGGTCAACTTTAC
TTGTTCCAAATTTACAATAAAGACTTCTCAAAAAAAAGCACGGGTAACGATAATTTACAC
ACTATGTATTTAAAGAACCTTTTCTCGGAAGAGAATTTAAAGGATATCGTATTGAAGTTG
AATGGAGAAGCGGAGATCTTCTTCCGTAAGTCCAGTATTAAAAACCCTATTATTCACAAG
AAGGGATCGATTTTAGTTAACCGCACATACGAGGCCGAAGAGAAGGACCAATTTGGGAAC
ATTCAAATTGTCCGCAAAAACATCCCTGAGAACATTTATCAAGAGCTTTATAAGTACTTT
AACGATAAGTCCGATAAGGAATTGTCAGATGAGGCGGCAAAGTTGAAGAATGTCGTGGGG
CATCATGAAGCTGCCACCAACATTGTGAAGGACTACCGCTACACTTACGACAAATACTTC
CTGCACATGCCCATTACGATCAATTTTAAGGCCAATAAGACAGGCTTTATTAACGACCGT
ATTCTTCAATATATCGCTAAGGAGAAGGACCTTCATGTGATTGGGATCGACCGCGGAGAA
CGTAATTTAATTTATGTGTCCGTCATCGATACGTGTGGAAATATCGTGGAACAGAAATCA
TTCAATATCGTGAATGGCTATGATTACCAGATCAAATTAAAACAGCAGGAGGGCGCTCGC
CAAATTGCGCGTAAGGAATGGAAAGAGATCGGAAAAATCAAAGAAATCAAAGAAGGATAT
TTGTCATTGGTGATCCATGAGATTTCAAAAATGGTAATTAAATATAATGCAATTATCGCA
ATGGAAGACCTGTCCTATGGTTTTAAGAAGGGTCGTTTCAAGGTAGAACGCCAAGTGTAT
CAAAAGTTCGAGACGATGCTGATCAATAAGCTGAATTATCTTGTGTTTAAGGACATTAGC
ATCACGGAAAATGGAGGGCTGTTGAAAGGCTATCAACTGACGTATATCCCTGACAAGCTG
AAAAATGTTGGCCATCAGTGCGGGTGCATTTTCTACGTCCCCGCGGCGTATACAAGCAAG
ATCGATCCTACTACGGGATTCGTAAATATTTTTAAATTCAAAGACTTAACCGTGGACGCC
AAGCGCGAATTCATTAAGAAGTTTGATAGCATTCGCTACGATTCAGAAAAAAATCTTTTC
TGTTTTACGTTCGATTACAACAATTTTATCACCCAGAACACAGTGATGAGCAAGTCATCC
TGGTCTGTCTATACCTACGGTGTCCGTATCAAACGCCGCTTCGTCAACGGACGCTTCTCT
AATGAATCTGATACCATTGACATCACCAAGGACATGGAAAAGACACTTGAGATGACAGAT
ATTAACTGGCGTGACGGACATGACCTGCGTCAGGACATCATCGATTATGAGATTGTTCAG
CATATCTTCGAGATCTTCCGCCTGACAGTACAAATGCGCAATTCACTGTCAGAACTTGAA
GACCGCGACTATGACCGCCTGATCTCTCCAGTATTAAATGAGAACAATATCTTTTATGAC
AGTGCTAAGGCCGGCGATGCCCTTCCGAAAGATGCTGATGCTAACGGAGCTTATTGTATT
GCATTAAAGGGTCTTTATGAGATCAAGCAAATTACCGAGAATTGGAAGGAGGATGGCAAA
TTCTCGCGCGACAAACTGAAAATCAGTAACAAGGACTGGTTCGATTTTATTCAGAATAAA
CGTTACCTG
SEQ ID NO: 32
ATGAATAACGGAACGAACAACTTCCAGAACTTCATCGGCATCAGTTCTTTACAAAAAACC
CTGCGTAACGCCCTTATTCCGACTGAGACAACACAACAGTTCATCGTTAAAAACGGAATT
ATCAAAGAGGACGAGTTGCGCGGCGAGAATCGCCAAATTTTGAAAGATATTATGGACGAC
TATTATCGTGGTTTTATTTCAGAAACACTGAGTTCGATTGACGATATCGATTGGACGAGC
CTGTTTGAGAAAATGGAAATCCAGTTGAAAAATGGCGATAATAAAGACACTTTAATCAAA
GAACAAACCGAGTATCGTAAAGCGATCCATAAAAAGTTCGCTAATGACGATCGTTTTAAG
AATATGTTCAGTGCGAAACTGATTTCAGACATTTTGCCCGAGTTCGTGATCCATAATAAC
AACTATTCCGCCTCGGAAAAGGAAGAAAAAACCCAGGTGATTAAGCTGTTCAGTCGCTTC
GCAACATCTTTCAAGGATTATTTCAAGAATCGCGCGAATTGCTTCAGTGCGGACGATATT
TCTAGTTCAAGCTGCCATCGTATCGTTAATGATAACGCGGAGATTTTTTTTAGCAATGCT
CTGGTGTACCGCCGCATTGTTAAGTCACTGTCCAACGATGATATTAACAAGATCTCAGGA
GACATGAAAGACTCGCTTAAAGAGATGAGTCTGGAAGAGATCTATTCTTATGAGAAGTAT
GGCGAGTTTATTACCCAAGAAGGAATCTCATTCTACAATGATATTTGTGGAAAGGTGAAC
AGCTTTATGAATCTTTACTGCCAAAAAAACAAGGAGAATAAGAATCTTTACAAACTTCAG
AAGTTACATAAACAGATTTTGTGTATTGCGGATACGTCTTATGAAGTCCCCTACAAATTT
GAATCGGATGAAGAGGTATACCAAAGTGTGAACGGATTCTTGGACAATATTTCTTCTAAA
CATATTGTTGAACGCTTACGTAAGATCGGGGATAACTACAATGGCTACAATCTTGACAAA
ATCTACATTGTTAGCAAATTCTACGAGAGTGTCAGCCAAAAGACGTACCGCGATTGGGAA
ACAATTAATACTGCGCTTGAGATTCACTATAATAACATTTTACCAGGCAACGGCAAGTCC
AAGGCGGATAAAGTTAAAAAAGCTGTTAAAAACGATTTGCAAAAATCTATCACAGAAATT
AACGAGTTAGTTAGTAACTACAAACTGTGCTCCGATGACAACATTAAGGCTGAGACGTAT
ATCCATGAGATCTCTCACATCTTAAACAATTTTGAAGCTCAAGAACTTAAGTACAATCCG
GAAATCCACCTGGTGGAATCCGAGCTGAAGGCTAGCGAACTGAAGAACGTATTGGACGTG
ATCATGAACGCGTTCCACTGGTGTTCTGTCTTTATGACGGAAGAGCTTGTCGACAAAGAT
AATAACTTTTACGCGGAACTTGAGGAAATTTACGATGAGATTTACCCAGTTATTTCATTG
TATAACCTTGTCCGTAATTACGTGACCCAAAAGCCTTATAGTACGAAAAAAATCAAATTA
AATTTTGGAATCCCAACACTGGCTGACGGTTGGAGCAAATCTAAGGAGTATTCTAATAAC
GCAATCATCTTAATGCGTGACAACCTGTATTATTTGGGTATCTTCAATGCCAAAAATAAG
CCTGACAAAAAGATTATCGAAGGAAATACTTCGGAGAATAAGGGGGATTACAAAAAAATG
ATTTACAATTTGCTGCCCGGGCCGAACAAGATGATCCCCAAAGTGTTCTTATCCTCGAAG
ACTGGTGTAGAAACATACAAGCCAAGCGCATACATTCTGGAGGGTTACAAGCAAAACAAA
CACATCAAATCTTCAAAAGACTTTGACATTACATTTTGCCATGATCTTATTGACTACTTC
AAAAACTGCATTGCTATTCACCCCGAGTGGAAGAACTTTGGGTTTGACTTCAGCGACACG
TCTACGTATGAGGACATCTCCGGGTTCTACCGTGAAGTTGAGTTACAAGGGTATAAGATT
GACTGGACGTATATTTCAGAGAAAGATATCGATCTTTTGCAGGAAAAGGGCCAGTTATAT
TTATTCCAGATTTACAACAAGGACTTTAGTAAGAAGTCAACAGGAAATGACAACTTGCAT
ACGATGTATTTGAAAAATCTTTTTTCTGAGGAAAATCTTAAGGACATCGTACTGAAATTG
AATGGCGAGGCTGAAATCTTCTTCCGTAAATCCTCCATTAAGAATCCCATTATCCACAAA
AAGGGGTCTATCCTGGTGAATCGTACCTACGAGGCAGAGGAGAAGGATCAATTCGGAAAT
ATTCAGATTGTTCGTAAGAACATCCCCGAGAACATTTATCAAGAATTGTATAAGTACTTT
AATGACAAATCTGACAAAGAGTTATCCGACGAAGCTGCGAAACTGAAAAACGTTGTTGGT
CACCACGAGGCCGCCACTAATATCGTAAAAGACTACCGTTATACCTATGACAAGTACTTT
TTGCACATGCCGATCACTATCAACTTCAAGGCGAATAAGACGGGCTTCATTAACGATCGT
ATCCTGCAATACATCGCCAAGGAGAAGGACCTTCACGTCATTGGGATTGACCGTGGTGAG
CGTAACCTGATTTATGTAAGCGTCATTGATACCTGCGGTAATATCGTCGAACAGAAAAGT
TTCAACATTGTAAATGGATATGACTATCAGATCAAACTTAAGCAGCAGGAGGGTGCACGC
CAGATTGCCCGCAAGGAATGGAAGGAGATTGGGAAGATTAAGGAAATTAAAGAAGGTTAC
TTATCACTGGTTATTCACGAGATCAGTAAAATGGTAATCAAATATAACGCGATCATTGCC
ATGGAGGATCTGAGCTATGGCTTTAAAAAGGGCCGTTTCAAAGTCGAGCGCCAGGTATAT
CAAAAGTTTGAAACAATGCTGATTAACAAATTAAACTATCTGGTTTTCAAAGATATTTCG
ATCACTGAAAATGGCGGGCTGTTGAAGGGATACCAACTTACATACATCCCTGACAAACTG
AAAAATGTCGGTCACCAATGTGGATGTATCTTTTATGTACCAGCAGCGTATACGAGCAAA
ATCGATCCAACTACGGGTTTTGTGAACATCTTTAAGTTCAAGGATTTGACAGTAGATGCC
AAACGCGAGTTCATTAAAAAATTTGATTCAATTCGCTACGATTCAGAGAAAAATCTTTTT
TGTTTCACGTTCGATTACAATAATTTCATTACGCAGAACACAGTAATGTCAAAGTCAAGC
TGGTCGGTCTACACGTATGGAGTCCGTATTAAACGTCGTTTTGTAAACGGCCGTTTCTCA
AATGAATCAGATACAATTGATATTACGAAGGATATGGAGAAGACATTAGAGATGACTGAC
ATTAACTGGCGCGACGGACATGATCTTCGTCAGGACATTATTGATTATGAGATTGTACAG
CATATCTTTGAGATCTTCCGCCTGACCGTTCAGATGCGCAATTCGTTGTCCGAGTTAGAA
GACCGCGATTACGACCGTTTAATCAGTCCCGTCTTAAACGAAAATAACATCTTCTACGAT
TCAGCCAAGGCAGGCGATGCCTTGCCAAAGGATGCTGACGCAAATGGCGCATACTGTATT
GCGTTGAAAGGCCTTTATGAAATCAAGCAAATTACCGAAAACTGGAAAGAAGACGGAAAA
TTCTCCCGTGATAAGTTGAAAATCTCTAATAAGGATTGGTTCGATTTCATCCAAAATAAA
CGCTATTTG
SEQ ID NO: 33
ATGAACAACGGAACTAATAATTTCCAAAATTTTATAGGCATCTCTTCTTTACAGAAGACT
CTTCGTAACGCCCTAATCCCGACTGAGACCACACAACAATTCATAGTGAAAAATGGGATC
ATTAAAGAAGACGAGCTGCGTGGGGAGAACAGGCAGATCCTAAAAGACATAATGGACGAT
TATTATAGAGGGTTCATCTCAGAGACATTATCTAGCATCGACGACATTGACTGGACCTCC
CTGTTTGAAAAAATGGAAATCCAGCTGAAGAATGGTGACAATAAAGACACATTAATAAAA
GAACAAACAGAGTACAGGAAAGCCATCCACAAGAAGTTCGCAAACGATGACAGATTCAAA
AATATGTTCAGTGCGAAGCTAATATCCGACATCTTACCAGAGTTTGTAATACACAATAAC
AATTACAGCGCGAGCGAAAAGGAAGAGAAAACGCAAGTAATTAAGCTTTTTAGTAGGTTC
GCTACCTCTTTCAAAGATTACTTCAAAAATCGTGCTAACTGCTTCTCAGCCGACGACATA
TCTTCAAGTTCCTGTCACCGTATCGTGAATGATAACGCTGAGATATTCTTCTCAAACGCC
CTTGTATACCGTAGGATCGTAAAGTCCTTATCTAACGATGATATAAACAAGATCAGTGGA
GACATGAAAGACAGCCTTAAAGAGATGTCTCTAGAAGAAATTTACTCCTATGAAAAGTAT
GGGGAGTTTATAACACAGGAGGGGATCAGCTTCTACAACGACATCTGCGGAAAGGTGAAC
AGTTTCATGAATCTTTACTGCCAGAAGAATAAAGAGAACAAAAATCTTTATAAGCTTCAA
AAGTTGCACAAACAAATACTGTGCATTGCCGATACATCATATGAGGTCCCCTATAAGTTC
GAATCTGATGAGGAAGTTTATCAATCTGTTAACGGCTTTCTAGACAATATCAGCTCAAAA
CACATCGTAGAAAGACTGAGGAAAATAGGTGATAATTATAATGGATACAACTTGGATAAA
ATATATATAGTCTCTAAATTTTACGAGTCAGTATCCCAGAAAACGTATAGGGATTGGGAG
ACCATCAACACGGCGTTAGAGATTCATTACAATAACATCTTACCGGGAAACGGAAAAAGT
AAGGCGGACAAAGTAAAGAAAGCCGTTAAAAATGACTTACAAAAGAGTATAACAGAAATA
AACGAACTAGTAAGCAACTACAAGCTTTGTTCCGATGATAATATCAAGGCCGAGACATAT
ATCCATGAGATCTCCCACATTCTAAACAATTTCGAAGCGCAAGAACTTAAATATAATCCC
GAAATCCACCTGGTGGAAAGTGAACTAAAGGCTAGTGAGTTAAAGAACGTTCTTGATGTT
ATCATGAACGCCTTCCATTGGTGCTCTGTTTTTATGACCGAGGAGTTGGTTGATAAAGAT
AATAATTTCTACGCTGAATTAGAGGAGATATACGACGAAATCTACCCAGTGATTTCACTA
TACAACTTGGTCAGGAACTATGTTACACAAAAGCCGTACAGCACTAAGAAAATTAAGCTA
AATTTCGGTATCCCCACGTTAGCCGACGGGTGGAGCAAGTCCAAAGAATATTCCAACAAT
GCGATTATTTTAATGCGTGACAATCTTTATTACCTTGGCATCTTCAATGCCAAAAACAAA
CCTGACAAAAAGATTATAGAAGGTAATACGTCCGAGAACAAAGGCGATTACAAGAAGATG
ATTTATAACCTACTGCCCGGACCAAACAAAATGATCCCCAAAGTTTTTCTTAGTTCTAAA
ACCGGCGTAGAGACGTATAAACCTTCTGCCTATATCTTAGAGGGATATAAGCAGAACAAA
CATATCAAATCTTCCAAGGACTTTGATATTACATTCTGCCACGATTTAATTGACTACTTC
AAAAATTGCATAGCGATACATCCGGAGTGGAAGAACTTTGGCTTCGACTTCAGTGATACA
TCCACCTATGAGGATATATCAGGCTTCTATCGTGAGGTCGAATTGCAAGGGTACAAAATC
GATTGGACGTATATATCCGAGAAAGACATAGACCTTCTTCAAGAAAAGGGGCAGTTATAT
TTATTCCAAATATACAACAAGGACTTCAGTAAGAAGTCAACAGGTAATGACAACTTACAC
ACCATGTACTTGAAAAATTTATTTTCTGAAGAAAACCTAAAGGACATTGTACTAAAACTG
AACGGGGAGGCAGAAATTTTTTTTAGAAAGAGCAGCATAAAAAACCCAATAATTCATAAG
AAAGGAAGCATTTTAGTTAATAGGACGTACGAGGCAGAGGAAAAGGACCAGTTTGGCAAT
ATCCAGATCGTAAGGAAAAATATTCCTGAAAACATATATCAGGAACTATATAAATACTTT
AACGACAAATCCGACAAAGAATTATCCGACGAGGCTGCAAAGCTGAAGAACGTCGTAGGG
CACCATGAGGCAGCGACTAATATTGTGAAAGACTATAGGTATACATACGACAAATACTTT
CTGCACATGCCCATCACGATTAACTTCAAGGCGAACAAGACGGGATTCATTAACGACCGT
ATATTACAATATATTGCTAAGGAGAAAGATCTGCATGTAATAGGTATCGACAGAGGCGAA
CGTAATTTAATCTACGTGTCCGTCATCGACACGTGCGGGAACATCGTAGAGCAAAAGAGT
TTTAATATAGTAAATGGCTATGATTACCAAATTAAGCTAAAGCAGCAAGAAGGAGCAAGA
CAGATAGCTAGGAAAGAATGGAAGGAGATAGGAAAAATAAAGGAGATCAAGGAGGGGTAT
CTTAGCCTAGTAATTCATGAAATATCTAAGATGGTTATCAAATACAACGCTATCATAGCG
ATGGAAGACTTATCTTATGGTTTCAAGAAAGGAAGGTTCAAAGTAGAGCGTCAAGTTTAT
CAAAAGTTCGAAACGATGTTGATTAATAAACTAAACTATTTGGTATTTAAAGATATATCT
ATCACCGAGAATGGTGGTCTACTAAAGGGTTACCAGCTTACATACATACCGGACAAACTT
AAAAACGTCGGACATCAGTGTGGATGCATTTTCTACGTTCCAGCTGCATATACCAGCAAG
ATCGACCCAACGACTGGGTTCGTAAATATTTTTAAATTCAAGGATTTGACTGTCGACGCC
AAAAGAGAGTTCATAAAAAAGTTCGATTCAATTAGGTACGACAGCGAAAAGAATTTGTTC
TGCTTTACTTTTGACTATAACAATTTCATTACTCAGAACACTGTAATGTCTAAGTCCTCT
TGGTCAGTCTATACTTATGGCGTTCGTATCAAACGTAGATTTGTTAACGGTAGATTCTCA
AATGAAAGTGATACAATAGATATCACGAAAGATATGGAGAAAACATTAGAAATGACAGAC
ATAAACTGGAGAGACGGACATGACTTGAGACAGGACATTATTGACTACGAGATCGTGCAG
CACATCTTTGAGATCTTTCGTTTGACCGTACAAATGCGTAACAGTTTATCTGAGCTTGAG
GACAGGGACTACGATAGATTGATATCACCTGTATTAAATGAGAATAACATCTTCTATGAT
TCCGCAAAAGCAGGCGACGCTCTACCCAAAGACGCTGATGCGAACGGTGCTTATTGCATA
GCTTTAAAGGGTTTGTATGAGATCAAACAGATAACAGAAAATTGGAAGGAAGATGGTAAG
TTCTCCCGTGACAAGCTTAAAATATCAAATAAGGACTGGTTCGATTTTATACAGAATAAG
CGTTATTA
SEQ ID NO: 34
ATGAACAATGGAACTAATAACTTCCAGAATTTCATTGGTATCTCCTCTTTACAAAAAACT
CTAAGAAACGCCCTAATTCCGACTGAAACTACACAGCAATTCATCGTCAAAAACGGGATC
ATTAAGGAGGATGAGTTGAGGGGTGAAAATCGTCAAATTCTTAAAGACATCATGGACGAC
TACTACAGGGGGTTCATCAGCGAGACGTTATCTAGTATAGACGATATAGACTGGACTTCA
CTGTTCGAGAAGATGGAAATCCAATTAAAAAATGGGGACAATAAAGATACACTTATAAAG
GAACAGACAGAGTATAGAAAGGCAATACACAAAAAGTTTGCCAACGACGATCGTTTCAAG
AACATGTTTAGTGCTAAATTGATTTCAGATATTCTGCCGGAATTTGTTATTCACAACAAT
AATTATAGCGCCAGTGAGAAAGAAGAAAAAACGCAGGTTATCAAACTGTTCAGTCGTTTC
GCTACATCTTTTAAGGATTACTTTAAAAACCGTGCAAATTGTTTTTCAGCCGACGATATT
AGTAGCAGCTCTTGTCACCGTATTGTTAATGATAATGCGGAGATTTTCTTTTCAAACGCA
TTGGTCTACAGGAGGATAGTCAAGTCCCTTTCAAATGACGACATTAATAAGATCTCAGGT
GACATGAAAGATTCCTTAAAGGAAATGTCCCTGGAAGAGATCTATTCCTATGAAAAGTAC
GGTGAGTTCATTACTCAAGAGGGTATAAGCTTTTACAATGACATATGTGGTAAGGTTAAT
AGCTTTATGAACCTGTATTGCCAGAAGAACAAAGAAAATAAGAATCTGTATAAGTTGCAA
AAGCTACACAAACAAATTTTGTGCATTGCCGATACATCATACGAGGTGCCATACAAATTC
GAGAGCGATGAGGAGGTTTATCAGAGCGTGAATGGATTCCTGGACAATATTAGTAGTAAG
CATATCGTGGAAAGGCTTAGAAAGATAGGTGACAATTACAATGGCTACAATCTGGATAAA
ATCTACATCGTCTCAAAATTCTATGAAAGTGTATCCCAGAAGACGTACCGTGATTGGGAA
ACTATCAACACCGCTCTGGAGATACATTACAACAATATACTTCCCGGAAACGGCAAGTCA
AAAGCCGACAAAGTCAAAAAAGCGGTCAAGAACGATTTACAAAAGTCTATCACTGAAATT
AATGAATTAGTTAGTAATTACAAACTGTGTAGTGATGATAATATTAAGGCAGAGACTTAC
ATACACGAAATTTCACACATTTTAAACAACTTCGAGGCACAGGAACTTAAATATAATCCT
GAAATTCACCTGGTTGAAAGTGAATTGAAAGCCAGCGAGCTAAAGAACGTTTTGGACGTA
ATCATGAACGCATTCCACTGGTGCTCTGTCTTTATGACAGAGGAACTAGTGGATAAGGAC
AATAATTTTTATGCGGAGCTGGAGGAAATATACGATGAGATATATCCCGTAATATCATTA
TATAATCTGGTAAGAAACTATGTGACTCAAAAGCCGTATAGCACCAAGAAAATTAAACTT
AATTTCGGCATACCCACTTTAGCGGACGGCTGGTCAAAATCCAAAGAGTATAGTAATAAT
GCCATCATCCTGATGCGTGACAACCTGTACTATTTAGGTATATTTAACGCCAAAAATAAA
CCCGACAAAAAGATTATAGAGGGCAACACCTCAGAGAACAAAGGTGATTATAAGAAGATG
ATTTACAACCTTTTACCCGGTCCTAATAAGATGATTCCCAAAGTCTTTCTATCTAGCAAA
ACTGGTGTTGAAACATACAAACCCTCAGCTTATATTTTAGAAGGGTATAAGCAGAATAAG
CATATTAAAAGCTCCAAAGATTTCGATATTACCTTTTGCCATGACTTGATAGACTATTTC
AAAAATTGTATTGCCATTCACCCTGAATGGAAAAACTTCGGATTTGACTTCTCTGACACA
TCCACCTACGAAGACATTTCAGGTTTTTACAGGGAAGTCGAGCTACAGGGTTATAAAATT
GATTGGACATACATCAGCGAGAAAGATATTGACCTACTTCAAGAAAAAGGGCAGCTATAC
CTGTTCCAGATATACAATAAAGACTTCAGTAAAAAAAGCACCGGGAACGATAATCTTCAC
ACAATGTACTTAAAAAATTTATTTAGTGAAGAGAATCTGAAGGATATAGTGCTGAAGTTA
AACGGGGAGGCAGAGATATTTTTTAGAAAATCTAGTATTAAGAATCCGATCATCCACAAG
AAGGGTTCTATCCTTGTTAATAGGACTTATGAGGCAGAAGAAAAAGACCAATTCGGCAAC
ATACAAATTGTCCGTAAAAATATCCCTGAGAACATTTATCAGGAACTATACAAGTACTTC
AATGATAAAAGCGACAAGGAGCTGAGCGACGAGGCTGCTAAGTTAAAGAATGTGGTGGGC
CACCATGAGGCAGCAACGAATATTGTGAAGGACTATCGTTATACCTACGATAAATACTTT
CTTCATATGCCGATCACCATTAATTTCAAGGCAAACAAAACTGGCTTCATTAACGATCGT
ATCTTACAATATATCGCAAAAGAGAAAGACCTTCACGTTATCGGGATCGATAGAGGCGAG
CGTAACCTAATTTATGTTTCTGTGATAGACACCTGTGGGAACATAGTCGAACAGAAATCA
TTTAATATTGTTAACGGCTACGATTATCAGATAAAGTTGAAGCAACAAGAGGGTGCACGT
CAAATAGCAAGGAAAGAATGGAAAGAAATAGGCAAGATTAAAGAAATAAAAGAAGGTTAT
TTATCCCTTGTAATACACGAAATTAGCAAAATGGTGATTAAATATAATGCGATCATTGCC
ATGGAGGATCTTTCTTACGGCTTCAAAAAGGGGAGATTCAAAGTCGAGAGGCAGGTGTAT
CAGAAGTTTGAGACCATGCTAATCAATAAACTAAATTATCTAGTATTCAAAGACATAAGC
ATCACCGAAAATGGCGGCTTGTTGAAGGGTTATCAATTGACCTACATCCCAGATAAACTA
AAAAACGTAGGGCATCAATGCGGATGTATATTTTACGTTCCAGCCGCATACACTTCCAAA
ATCGATCCAACTACGGGTTTTGTGAACATCTTCAAATTCAAAGACTTGACTGTCGATGCT
AAGAGGGAGTTTATCAAGAAATTTGACTCCATTAGATACGACAGTGAGAAGAATCTGTTC
TGTTTTACCTTTGATTATAACAACTTTATAACTCAAAACACAGTCATGAGTAAGTCATCT
TGGTCAGTGTATACGTATGGTGTGAGGATTAAAAGGAGGTTTGTTAACGGGAGATTTTCC
AATGAAAGTGATACAATAGATATAACCAAGGACATGGAAAAGACTCTTGAAATGACCGAC
ATTAACTGGAGAGATGGCCACGACTTACGTCAAGATATAATCGATTACGAGATAGTGCAA
CATATCTTTGAGATATTTAGGCTTACTGTCCAAATGCGTAACTCATTAAGTGAGTTGGAG
GACAGGGATTACGATAGGCTAATAAGTCCTGTTCTTAACGAAAACAATATATTCTACGAT
TCAGCAAAGGCGGGAGACGCCCTGCCCAAGGACGCGGATGCTAACGGCGCATACTGTATT
GCCCTGAAAGGCTTGTACGAGATAAAACAGATCACGGAGAACTGGAAAGAAGATGGAAAA
TTCAGTCGTGACAAGTTAAAAATTAGTAACAAAGACTGGTTCGACTTTATTCAGAACAAG
AGATATCTG
SEQ ID NO: 35
ATGAACAACGGAACCAATAACTTTCAAAACTTTATAGGCATCTCCAGTCTACAGAAGACA
CTACGTAACGCTTTGATACCAACTGAGACCACGCAGCAGTTTATCGTCAAGAACGGTATT
ATAAAGGAAGACGAGCTAAGGGGGGAAAACCGTCAGATCTTAAAGGACATCATGGATGAC
TACTACAGAGGCTTCATAAGTGAGACTTTGTCTAGTATAGACGACATCGACTGGACCAGT
TTATTTGAGAAGATGGAAATTCAGTTAAAGAACGGGGACAATAAAGACACACTAATTAAA
GAGCAGACCGAATACAGAAAAGCTATACACAAAAAGTTTGCCAACGATGATAGATTCAAA
AATATGTTTTCAGCAAAATTGATTTCCGACATATTGCCAGAATTCGTAATCCATAATAAC
AATTATTCTGCAAGTGAGAAGGAAGAGAAGACCCAAGTAATCAAGCTGTTTTCCCGTTTT
GCTACGAGTTTCAAAGATTATTTCAAGAATAGGGCTAATTGTTTCTCCGCGGACGACATA
AGTAGCAGTTCCTGTCACAGGATTGTGAACGATAATGCTGAGATATTTTTTTCCAATGCC
CTAGTGTATAGGAGAATAGTTAAAAGCTTAAGCAACGACGATATCAATAAAATTTCAGGG
GACATGAAGGACAGCTTAAAGGAAATGAGTTTGGAGGAGATTTACAGTTATGAAAAATAC
GGAGAGTTTATAACTCAGGAAGGCATCTCTTTCTATAATGATATCTGTGGGAAGGTAAAC
TCCTTCATGAATTTATATTGCCAGAAGAATAAGGAAAACAAAAATCTTTACAAGCTTCAA
AAGTTACATAAGCAGATCTTATGTATTGCCGACACGAGTTATGAAGTGCCTTATAAATTC
GAGAGTGATGAGGAAGTGTATCAGTCTGTTAACGGATTCCTAGATAATATAAGTTCCAAA
CATATAGTCGAGAGGCTGAGGAAGATTGGCGATAACTATAATGGATATAATCTTGACAAA
ATCTATATAGTCTCTAAATTTTATGAAAGCGTCAGCCAGAAGACATATAGAGATTGGGAA
ACTATAAACACAGCCCTTGAAATACATTACAATAACATCCTACCCGGCAATGGTAAGTCT
AAGGCAGACAAAGTTAAAAAAGCAGTAAAGAATGACTTACAGAAGTCAATCACGGAGATA
AATGAGTTGGTCAGTAACTACAAATTATGCTCCGACGATAATATTAAGGCCGAAACATAT
ATACACGAGATAAGTCATATATTAAACAATTTCGAAGCCCAGGAGTTAAAATATAACCCT
GAAATTCATCTGGTCGAAAGTGAGTTAAAGGCCAGTGAGTTAAAGAATGTACTTGACGTA
ATTATGAATGCTTTTCATTGGTGCTCCGTGTTCATGACCGAGGAGTTAGTAGATAAAGAC
AATAACTTTTACGCCGAACTTGAAGAGATATACGACGAGATTTATCCGGTAATCAGCTTG
TACAACTTAGTTAGAAATTATGTAACACAGAAGCCTTACTCTACTAAAAAAATAAAACTG
AACTTTGGTATCCCAACTCTTGCAGATGGTTGGAGTAAAAGCAAGGAATATAGCAACAAT
GCGATCATCTTGATGAGAGACAACTTGTACTATTTGGGAATCTTCAACGCGAAAAATAAA
CCCGACAAAAAAATCATCGAAGGGAATACCTCTGAGAATAAAGGTGACTATAAGAAAATG
ATTTACAATCTACTTCCTGGTCCTAATAAAATGATCCCGAAAGTGTTTCTTAGTTCTAAG
ACTGGTGTCGAGACGTACAAACCTAGCGCGTACATCTTAGAAGGGTACAAGCAGAATAAA
CACATCAAATCAAGCAAAGACTTCGATATTACTTTTTGCCATGACTTGATAGACTACTTT
AAAAACTGCATAGCAATCCACCCGGAGTGGAAAAACTTTGGCTTTGATTTCTCTGACACC
TCTACATATGAGGACATATCTGGTTTTTACCGTGAGGTTGAATTGCAGGGATACAAAATT
GACTGGACTTACATATCTGAAAAAGATATCGATCTATTGCAGGAGAAAGGCCAGCTTTAC
CTTTTCCAGATCTATAATAAGGACTTCTCTAAGAAGTCTACAGGGAATGATAATTTGCAC
ACTATGTACTTAAAAAATCTGTTTTCCGAGGAAAACTTGAAAGACATTGTTTTAAAGTTG
AACGGAGAAGCTGAAATATTTTTCAGAAAGAGCTCCATAAAAAACCCGATCATTCATAAG
AAGGGATCTATCCTGGTTAACAGAACGTACGAAGCGGAAGAAAAAGACCAATTCGGAAAC
ATTCAAATTGTTAGAAAGAATATCCCTGAGAACATCTACCAGGAGTTATATAAGTATTTT
AATGATAAGTCAGATAAGGAACTATCTGACGAAGCGGCGAAGCTTAAAAATGTTGTAGGA
CACCATGAGGCTGCTACAAATATAGTCAAGGACTACCGTTATACCTACGATAAGTACTTT
CTACACATGCCCATTACCATCAATTTTAAAGCTAATAAAACGGGTTTTATCAACGATCGT
ATCCTACAATATATTGCGAAAGAGAAGGATTTGCATGTCATTGGCATTGATAGAGGTGAG
AGGAACCTAATATACGTATCCGTGATTGATACGTGCGGGAACATAGTTGAACAGAAATCA
TTTAATATAGTTAATGGGTACGACTATCAGATTAAGCTAAAGCAACAAGAAGGCGCCAGG
CAAATTGCCCGTAAAGAATGGAAAGAGATCGGGAAGATCAAGGAAATAAAAGAAGGATAC
CTTTCCCTGGTCATCCATGAAATTAGCAAAATGGTGATTAAGTACAATGCCATAATCGCG
ATGGAGGACTTAAGCTACGGGTTCAAAAAGGGGAGGTTTAAGGTGGAGAGGCAAGTGTAC
CAGAAATTTGAGACCATGCTAATCAACAAACTGAACTACCTAGTTTTTAAGGACATTTCA
ATTACAGAGAATGGAGGACTTTTAAAGGGTTACCAACTAACGTATATACCAGATAAGTTG
AAAAATGTCGGTCACCAGTGTGGCTGCATCTTTTACGTTCCCGCCGCTTATACATCTAAA
ATTGATCCAACCACAGGCTTTGTAAATATCTTTAAATTCAAAGATTTAACTGTGGATGCA
AAAAGAGAGTTTATCAAGAAATTCGATAGCATTCGTTATGATAGCGAGAAGAACCTGTTC
TGCTTTACTTTCGACTATAACAACTTTATAACTCAAAACACCGTGATGTCAAAAAGCTCA
TGGTCAGTCTACACCTATGGTGTAAGGATTAAAAGGCGTTTCGTGAATGGGAGATTCTCC
AATGAAAGTGACACGATCGACATAACAAAGGACATGGAGAAGACACTAGAGATGACTGAT
ATTAATTGGAGAGACGGACACGATCTGCGTCAAGATATAATTGATTATGAGATAGTACAG
CACATATTTGAGATCTTCCGTTTGACTGTCCAAATGCGTAATTCCCTTTCTGAGCTGGAA
GATAGGGACTATGATAGATTAATATCCCCTGTACTAAATGAGAACAACATTTTCTATGAT
AGTGCAAAAGCCGGGGATGCATTGCCGAAAGACGCTGACGCTAATGGGGCGTACTGTATA
GCTTTAAAGGGGCTTTACGAAATAAAGCAGATAACCGAAAACTGGAAGGAAGATGGCAAA
TTCTCAAGGGACAAACTTAAGATCTCTAACAAGGATTGGTTCGATTTTATACAAAACAAA
CGTTATTTG
SEQ ID NO: 36
ATGAATAATGGTACAAACAACTTTCAGAATTTCATTGGGATCTCTAGCTTACAGAAGACC
CTGAGGAATGCGTTGATTCCAACTGAAACAACCCAGCAATTCATCGTGAAAAATGGGATA
ATCAAAGAGGATGAGTTAAGGGGTGAAAACCGTCAAATATTGAAGGATATTATGGACGAC
TACTACCGTGGATTCATCTCAGAGACGTTGAGCAGCATTGACGACATAGACTGGACTAGC
CTTTTCGAGAAGATGGAAATTCAGTTAAAGAACGGAGATAACAAAGATACACTAATCAAG
GAACAGACAGAATACAGAAAAGCAATTCATAAGAAATTCGCTAATGACGATCGTTTTAAA
AACATGTTCTCTGCAAAATTAATTAGCGACATTCTGCCGGAATTCGTTATACATAATAAT
AACTACAGTGCTTCTGAAAAGGAAGAGAAAACTCAGGTAATAAAACTGTTCTCTCGTTTT
GCCACATCCTTCAAAGACTACTTTAAAAATAGAGCGAACTGCTTTAGCGCCGACGATATT
AGTTCTTCCTCATGCCACAGGATTGTCAACGATAATGCAGAGATATTCTTTTCTAACGCA
CTAGTCTACAGAAGGATTGTAAAGTCTTTGTCAAATGATGACATAAACAAGATTAGTGGA
GATATGAAAGACTCTCTAAAGGAAATGAGCCTTGAGGAGATATACTCTTATGAAAAGTAC
GGTGAGTTTATTACCCAAGAAGGCATTAGTTTCTATAATGACATTTGTGGAAAAGTTAAC
AGTTTTATGAATCTATACTGTCAAAAAAATAAGGAGAATAAAAATCTTTATAAGTTGCAA
AAACTGCATAAGCAGATATTATGTATAGCAGACACGAGCTATGAGGTACCGTACAAGTTC
GAGAGCGATGAGGAAGTCTACCAATCTGTCAACGGATTTTTGGACAACATTTCTTCAAAA
CATATTGTGGAGAGGCTTAGGAAAATAGGCGACAATTATAATGGATATAACTTAGATAAG
ATATATATTGTTTCCAAATTCTACGAATCTGTAAGCCAGAAGACATACAGAGATTGGGAA
ACGATAAACACAGCCCTTGAAATTCACTATAACAACATACTACCTGGAAACGGCAAATCA
AAGGCCGACAAAGTTAAGAAGGCCGTAAAGAATGATTTACAGAAGAGCATAACGGAGATC
AATGAGCTGGTGTCTAACTATAAATTGTGTAGCGATGACAACATAAAAGCCGAGACTTAC
ATTCACGAAATTTCACACATACTTAACAACTTTGAAGCTCAGGAATTAAAGTATAATCCC
GAAATACACCTTGTGGAGTCCGAACTAAAGGCTAGTGAGCTTAAGAACGTCCTAGACGTA
ATTATGAATGCCTTCCACTGGTGTAGTGTTTTTATGACCGAGGAACTTGTTGACAAAGAT
AATAATTTTTATGCAGAACTAGAAGAGATATACGATGAAATATACCCGGTGATCAGTTTG
TACAATCTTGTCAGGAACTATGTGACACAAAAGCCCTATTCAACAAAGAAAATAAAACTT
AATTTCGGAATTCCTACGTTAGCTGATGGCTGGTCTAAATCCAAGGAATACAGCAACAAC
GCTATAATTCTGATGAGAGATAACTTGTACTATCTAGGCATCTTCAATGCCAAAAATAAG
CCTGATAAGAAGATTATAGAGGGCAACACTTCAGAGAACAAGGGCGACTACAAGAAAATG
ATCTATAACCTATTGCCTGGCCCAAACAAGATGATTCCGAAGGTCTTCCTATCATCCAAG
ACCGGCGTTGAGACATACAAGCCATCAGCGTATATTTTAGAGGGGTACAAACAAAACAAG
CACATAAAGTCTAGTAAAGACTTCGATATAACATTTTGTCATGACTTAATTGACTACTTT
AAGAATTGCATCGCTATACACCCGGAATGGAAGAATTTCGGCTTCGACTTCTCTGATACA
TCTACCTACGAGGACATTAGCGGGTTTTACCGTGAAGTCGAATTACAAGGGTATAAGATA
GATTGGACGTACATCTCTGAGAAAGACATAGACTTGCTTCAGGAAAAGGGCCAGTTGTAT
CTATTCCAAATATACAATAAGGATTTTTCCAAGAAATCTACGGGTAATGACAATCTTCAC
ACAATGTATCTTAAGAACCTTTTCTCAGAAGAGAACCTGAAGGACATTGTCTTAAAACTA
AATGGCGAAGCTGAGATTTTTTTCAGGAAGTCTTCAATTAAGAACCCGATAATCCACAAG
AAGGGGAGTATTCTTGTGAATAGAACTTACGAGGCCGAAGAAAAAGACCAATTTGGTAAC
ATCCAGATAGTCAGAAAGAACATTCCAGAGAACATCTACCAAGAGCTATACAAATATTTC
AACGACAAGTCCGATAAGGAACTGTCCGATGAGGCAGCCAAGTTGAAGAATGTCGTGGGT
CATCATGAAGCTGCTACTAACATTGTCAAGGACTATCGTTATACTTACGACAAGTATTTC
CTACACATGCCGATAACAATTAATTTCAAGGCTAACAAAACAGGCTTTATCAACGATCGT
ATCTTGCAGTACATAGCTAAGGAAAAGGATTTGCATGTGATTGGCATTGATAGAGGGGAG
CGTAACTTGATATATGTGTCTGTCATAGACACGTGTGGCAACATCGTCGAACAGAAATCA
TTCAACATAGTAAACGGCTACGATTACCAAATTAAGCTGAAACAGCAAGAGGGTGCACGT
CAAATTGCGCGTAAAGAGTGGAAAGAAATTGGTAAAATCAAGGAAATTAAAGAAGGCTAC
TTGTCTCTTGTTATACATGAAATTTCCAAGATGGTTATAAAGTATAACGCGATAATTGCT
ATGGAAGACTTATCATACGGGTTTAAAAAGGGGAGGTTCAAGGTAGAGAGGCAGGTCTAT
CAAAAGTTCGAGACGATGTTGATTAATAAACTAAACTATCTAGTGTTCAAAGATATCAGC
ATTACGGAGAACGGGGGGCTACTGAAAGGATATCAACTAACGTACATTCCCGATAAGTTA
AAGAACGTTGGTCATCAATGTGGTTGCATCTTCTACGTGCCTGCTGCCTATACGTCCAAA
ATAGATCCAACTACTGGATTTGTTAACATCTTTAAATTCAAAGATTTAACCGTAGACGCC
AAAAGGGAATTTATAAAAAAATTTGACAGCATCCGTTACGATAGCGAAAAGAATCTGTTC
TGTTTTACTTTCGACTACAATAATTTCATCACGCAAAATACGGTAATGTCTAAGTCAAGT
TGGAGCGTCTACACGTATGGAGTCAGGATCAAGAGGCGTTTCGTAAATGGAAGATTCTCT
AATGAGTCAGATACTATAGACATCACGAAAGATATGGAGAAAACCTTGGAGATGACGGAT
ATTAACTGGCGTGATGGACACGATTTAAGACAGGACATTATTGACTATGAGATTGTGCAA
CACATCTTCGAAATATTCCGTCTAACAGTCCAAATGAGGAATAGCCTAAGTGAATTGGAG
GACCGTGATTACGATAGGCTTATAAGTCCTGTCCTTAACGAAAACAATATTTTCTATGAT
AGTGCTAAGGCGGGGGACGCACTGCCTAAAGACGCAGATGCTAACGGGGCATACTGCATT
GCGTTAAAGGGTCTGTACGAAATCAAGCAGATTACGGAAAACTGGAAAGAGGATGGCAAG
TTTAGCAGAGATAAGTTGAAGATAAGTAACAAAGATTGGTTTGACTTTATTCAGAATAAA
AGGTATTTA
SEQ ID NO: 37
ATGAATAACGGCACTAATAATTTCCAGAATTTCATCGGCATTAGCAGCTTACAAAAGACG
TTGAGGAATGCCTTAATACCCACAGAAACTACTCAACAATTTATAGTGAAGAATGGGATA
ATTAAGGAAGACGAGTTGAGAGGTGAAAATAGGCAAATCTTGAAAGACATTATGGATGAC
TACTACAGGGGCTTCATTAGTGAAACGTTGTCTTCAATAGATGACATTGATTGGACTTCT
TTGTTTGAGAAGATGGAAATACAGTTAAAGAACGGCGACAATAAGGATACACTTATCAAA
GAGCAAACAGAATATAGAAAAGCAATTCACAAAAAGTTTGCTAACGATGATAGGTTCAAG
AACATGTTTAGCGCTAAACTAATATCAGACATCCTTCCCGAGTTCGTTATTCATAACAAT
AACTATAGTGCAAGTGAAAAAGAGGAGAAGACACAGGTGATTAAGCTGTTCTCCAGATTC
GCGACTTCTTTCAAAGATTACTTCAAAAACAGAGCCAACTGTTTTTCAGCTGACGATATC
TCTAGTAGTAGTTGTCACCGTATAGTGAACGATAACGCTGAGATCTTCTTTAGCAATGCA
TTAGTGTATAGAAGGATAGTTAAGTCTCTAAGCAATGATGATATCAATAAAATTTCCGGA
GACATGAAGGACTCCCTAAAGGAAATGTCCTTAGAAGAGATCTACTCATATGAGAAATAC
GGGGAATTTATTACGCAGGAAGGGATCTCCTTTTACAATGACATATGCGGGAAGGTCAAC
TCTTTCATGAACTTATACTGCCAAAAGAACAAGGAGAACAAGAATTTATATAAACTTCAG
AAACTTCACAAACAAATACTGTGCATAGCCGATACCTCATATGAGGTTCCTTACAAATTT
GAATCAGATGAAGAGGTATACCAATCCGTTAACGGCTTTCTTGACAATATTAGCTCAAAG
CACATCGTGGAGAGGTTGAGAAAGATTGGTGATAATTATAATGGCTACAATCTAGATAAG
ATATATATTGTTAGCAAGTTCTACGAGTCTGTGTCCCAAAAAACATATAGGGATTGGGAG
ACAATTAATACTGCTCTAGAAATCCATTACAACAACATCCTTCCTGGAAATGGCAAGAGT
AAGGCCGACAAAGTCAAGAAAGCAGTGAAAAATGATCTGCAAAAATCAATTACTGAGATA
AACGAGCTAGTATCTAATTACAAGCTTTGTAGCGACGATAACATTAAGGCAGAAACGTAC
ATACACGAGATTAGTCACATCTTAAATAATTTTGAAGCCCAAGAACTGAAATATAACCCT
GAGATACACCTTGTTGAATCCGAGTTAAAGGCGTCTGAACTAAAAAACGTGTTAGACGTT
ATTATGAATGCCTTCCACTGGTGTAGCGTCTTTATGACTGAGGAGTTGGTTGATAAGGAT
AATAACTTTTACGCTGAATTGGAAGAAATTTATGACGAAATCTATCCTGTTATTTCTCTA
TATAATTTGGTGAGAAATTACGTAACGCAAAAGCCCTATAGTACGAAAAAAATAAAACTA
AATTTCGGGATCCCTACCCTAGCCGACGGTTGGTCTAAATCCAAGGAGTACTCAAACAAT
GCAATAATATTGATGAGGGACAACCTGTACTACCTAGGCATATTTAATGCCAAAAATAAG
CCCGATAAAAAGATTATAGAAGGGAACACGTCAGAAAATAAAGGAGACTATAAGAAAATG
ATCTACAACCTTTTGCCCGGCCCCAATAAAATGATCCCGAAGGTCTTCCTAAGTAGCAAG
ACTGGCGTAGAGACCTACAAACCATCTGCATACATTTTGGAGGGGTACAAGCAAAACAAG
CACATAAAGAGTAGTAAGGATTTTGACATTACATTCTGCCATGACTTAATTGACTACTTT
AAAAATTGCATCGCAATTCACCCTGAATGGAAAAATTTTGGATTTGATTTCTCTGATACT
TCAACATATGAGGATATTTCAGGGTTCTACAGGGAGGTCGAACTACAGGGTTACAAAATA
GACTGGACGTATATTTCTGAGAAAGATATAGATTTGCTTCAGGAAAAGGGTCAGCTATAT
CTGTTCCAGATATATAATAAGGACTTCTCCAAAAAGAGTACCGGAAATGATAATCTGCAC
ACAATGTACTTAAAAAACTTGTTCTCTGAGGAGAATCTAAAAGACATCGTACTAAAACTT
AACGGGGAGGCCGAAATTTTTTTTAGGAAGTCCAGCATCAAGAACCCGATTATTCATAAA
AAAGGTAGCATTTTGGTGAACCGTACTTATGAGGCGGAAGAAAAAGACCAATTCGGTAAT
ATTCAAATCGTTAGAAAGAACATCCCTGAGAACATTTATCAGGAACTATACAAATACTTT
AACGACAAATCAGATAAGGAGCTTTCTGATGAGGCAGCTAAATTGAAAAATGTAGTGGGA
CATCACGAAGCAGCCACTAACATAGTGAAGGACTACAGATACACATACGATAAGTACTTC
CTGCACATGCCTATTACAATTAACTTTAAAGCAAATAAAACAGGGTTTATTAACGACAGA
ATCTTACAGTATATTGCCAAAGAAAAGGATCTGCATGTGATAGGAATAGACAGAGGAGAA
AGAAACCTGATATACGTCTCCGTGATTGATACATGTGGGAACATAGTAGAACAGAAGTCC
TTTAACATTGTTAATGGGTACGATTATCAAATTAAATTAAAACAACAAGAAGGAGCACGT
CAAATAGCTAGGAAAGAATGGAAAGAGATAGGAAAAATTAAGGAAATTAAGGAGGGTTAC
CTGTCCCTTGTAATTCATGAAATATCCAAAATGGTAATTAAATATAACGCGATCATCGCG
ATGGAAGATCTAAGCTACGGGTTCAAAAAAGGCAGGTTTAAGGTGGAGAGGCAAGTTTAC
CAAAAGTTCGAGACAATGTTGATTAATAAGTTAAACTACTTAGTTTTCAAAGATATCTCC
ATAACCGAGAATGGCGGGCTTTTAAAAGGGTACCAACTAACATATATCCCGGATAAATTG
AAGAACGTTGGACACCAGTGTGGCTGCATATTTTATGTACCCGCTGCGTATACTTCTAAA
ATTGACCCGACCACCGGGTTTGTAAACATATTCAAGTTTAAGGACCTAACAGTTGACGCC
AAACGTGAGTTCATCAAGAAGTTCGATAGTATAAGGTATGACTCTGAGAAGAACCTTTTC
TGCTTCACGTTTGACTATAATAATTTCATCACCCAAAATACAGTTATGTCAAAAAGCTCT
TGGTCAGTATATACGTATGGCGTAAGGATTAAGCGTAGGTTCGTGAACGGTAGATTTTCC
AACGAGTCAGATACTATTGATATTACCAAGGATATGGAGAAGACATTAGAAATGACAGAT
ATAAATTGGAGGGATGGGCACGATCTAAGGCAAGATATCATTGATTACGAAATTGTTCAG
CACATATTCGAGATATTCCGTCTTACAGTACAAATGCGTAACAGCTTGTCTGAGTTGGAA
GATCGTGACTATGACAGGTTGATATCACCGGTCTTGAACGAGAACAATATATTCTACGAC
AGCGCTAAGGCGGGAGACGCTCTGCCTAAAGACGCAGATGCCAATGGGGCGTACTGCATT
GCCTTAAAAGGCTTATACGAGATTAAACAGATCACAGAGAACTGGAAAGAGGACGGCAAG
TTTTCTAGAGATAAATTGAAAATCTCAAACAAAGACTGGTTCGATTTCATCCAAAACAAA
AGATACCTT
SEQ ID NO: 38
ATGAACAATGGAACTAACAACTTCCAGAACTTTATCGGCATCTCTTCCCTCCAAAAGACA
CTGAGAAATGCACTGATCCCAACCGAAACGACTCAACAATTTATTGTTAAGAACGGCATC
ATAAAAGAAGACGAGCTTCGCGGCGAGAACCGCCAGATACTTAAGGATATTATGGACGAT
TATTACCGAGGCTTTATCAGCGAAACTCTTAGCTCTATTGATGATATCGACTGGACCTCC
CTCTTCGAAAAAATGGAGATACAGCTCAAGAACGGCGATAATAAAGACACCTTGATAAAG
GAACAGACTGAGTACAGGAAAGCGATCCACAAGAAATTCGCGAACGACGACAGGTTTAAA
AACATGTTCTCTGCAAAATTGATATCCGACATCTTGCCGGAATTTGTGATACACAACAAT
AACTATAGCGCTTCAGAGAAAGAAGAGAAGACCCAAGTAATCAAGTTGTTCAGCCGCTTC
GCAACGTCTTTTAAAGATTACTTTAAGAACCGGGCCAATTGTTTCTCCGCGGATGATATT
AGCTCATCAAGTTGCCATCGAATTGTCAATGATAATGCGGAGATCTTCTTCAGCAATGCG
CTGGTCTACAGACGAATCGTAAAAAGTCTTTCAAATGACGACATCAATAAGATTAGTGGA
GATATGAAGGATTCCCTTAAGGAAATGAGTCTTGAAGAAATATACTCATACGAAAAGTAC
GGGGAATTTATTACCCAGGAGGGGATCTCCTTCTATAACGACATCTGTGGAAAAGTAAAC
TCATTCATGAACCTGTACTGTCAGAAAAACAAAGAAAACAAAAATCTGTATAAACTCCAA
AAATTGCACAAGCAAATATTGTGTATAGCGGACACATCATACGAGGTTCCATATAAGTTC
GAAAGTGATGAAGAAGTCTACCAATCAGTGAATGGGTTTCTGGACAACATTAGTTCCAAG
CACATAGTTGAACGACTGCGAAAGATTGGTGACAATTACAACGGCTATAATTTGGACAAG
ATTTATATAGTTAGCAAATTTTATGAATCCGTATCACAAAAGACTTATAGAGACTGGGAA
ACAATCAACACGGCACTTGAGATCCATTATAACAATATTCTTCCAGGGAACGGCAAAAGC
AAGGCTGATAAGGTAAAAAAGGCCGTTAAGAATGATCTTCAAAAATCCATAACGGAGATC
AACGAACTTGTAAGTAACTACAAATTGTGCTCTGACGACAATATAAAGGCTGAAACGTAT
ATTCACGAGATTAGCCATATCCTGAATAACTTTGAGGCCCAAGAACTCAAGTATAACCCG
GAAATACATTTGGTAGAAAGCGAGCTTAAAGCGAGTGAGCTGAAAAACGTCCTCGATGTG
ATCATGAATGCTTTCCACTGGTGTAGTGTCTTTATGACTGAGGAGTTGGTTGATAAAGAC
AATAATTTCTACGCTGAACTGGAAGAAATTTACGACGAAATCTATCCAGTGATCTCCCTC
TATAACCTCGTTCGAAACTACGTGACGCAGAAACCTTATTCTACAAAGAAAATTAAGTTG
AACTTCGGCATTCCTACACTTGCTGACGGATGGTCCAAATCCAAAGAGTACTCAAACAAC
GCAATCATCCTCATGCGGGATAACCTTTATTATTTGGGCATTTTCAACGCCAAAAACAAA
CCTGATAAAAAGATAATTGAAGGCAATACGAGTGAGAACAAGGGCGACTACAAAAAAATG
ATATATAACTTGTTGCCAGGCCCCAACAAGATGATTCCTAAAGTTTTTCTGTCTTCTAAG
ACTGGAGTTGAAACTTACAAACCCTCCGCCTACATTCTTGAAGGGTATAAACAGAATAAG
CACATAAAGTCCTCAAAGGATTTCGACATTACGTTTTGCCATGACCTCATCGACTATTTC
AAGAACTGTATCGCCATACATCCGGAGTGGAAGAATTTTGGATTTGATTTCTCCGACACA
TCTACCTATGAAGACATAAGCGGTTTCTACCGGGAGGTCGAGCTTCAGGGCTATAAGATA
GATTGGACATACATTAGTGAAAAAGATATCGATCTTCTGCAAGAAAAGGGACAACTTTAC
CTTTTTCAGATTTATAATAAAGACTTTTCAAAAAAGTCCACAGGGAACGATAATCTGCAC
ACCATGTATCTCAAGAATCTGTTTAGTGAAGAAAACCTTAAAGACATAGTTTTGAAGCTT
AACGGAGAGGCTGAGATTTTTTTTAGAAAGTCCTCAATTAAAAACCCTATAATACACAAG
AAAGGCTCTATTCTTGTTAACAGGACATATGAAGCCGAGGAGAAAGATCAGTTTGGCAAT
ATCCAGATTGTTCGCAAGAATATCCCGGAAAATATATATCAGGAGCTGTATAAATACTTT
AACGACAAGAGCGACAAGGAGCTGAGTGACGAGGCCGCGAAGCTTAAGAATGTAGTAGGT
CACCACGAAGCAGCCACCAATATCGTCAAAGACTATAGGTACACGTACGACAAGTACTTT
TTGCACATGCCTATAACTATAAACTTCAAAGCTAATAAAACTGGGTTTATTAATGACAGG
ATTCTCCAATACATCGCTAAAGAGAAGGATCTGCATGTAATTGGCATAGACAGAGGTGAG
AGAAACTTGATATATGTCAGCGTAATAGACACATGTGGCAATATCGTGGAACAGAAGTCT
TTTAACATCGTCAATGGTTACGACTACCAAATTAAGTTGAAACAGCAGGAAGGCGCACGA
CAGATCGCACGAAAGGAATGGAAAGAGATAGGCAAAATAAAAGAAATAAAGGAGGGCTAT
CTCAGTCTCGTTATACACGAAATTTCAAAAATGGTTATTAAGTACAATGCAATCATAGCG
ATGGAGGATCTCAGTTATGGGTTCAAAAAGGGTCGGTTTAAAGTTGAGCGCCAAGTGTAC
CAAAAGTTCGAGACAATGCTGATTAACAAGCTGAACTACCTCGTCTTCAAAGATATAAGT
ATTACGGAGAACGGTGGCCTTCTTAAAGGCTATCAACTTACTTACATCCCGGACAAGCTC
AAAAACGTAGGGCACCAATGCGGGTGTATTTTCTATGTGCCTGCGGCATATACGTCAAAG
ATTGACCCAACCACAGGATTCGTAAACATATTCAAGTTTAAGGACCTCACCGTTGATGCG
AAAAGGGAGTTCATTAAAAAATTTGATTCTATTCGATATGATAGTGAGAAAAATCTCTTT
TGTTTCACATTTGACTATAATAATTTTATTACTCAGAATACTGTCATGAGCAAGTCATCT
TGGTCAGTGTACACATACGGGGTGCGGATCAAACGCAGGTTCGTCAATGGTCGCTTCTCA
AACGAATCAGACACCATTGACATCACAAAGGACATGGAAAAAACCCTTGAGATGACCGAC
ATTAATTGGCGCGATGGTCATGATCTGCGGCAAGACATCATAGACTACGAAATCGTCCAA
CACATCTTTGAGATCTTTCGCTTGACGGTCCAAATGCGGAACTCCCTGTCCGAGCTCGAG
GATAGAGATTATGATCGGCTGATATCTCCCGTGCTTAATGAAAATAACATCTTCTACGAC
TCCGCCAAGGCGGGTGATGCCCTGCCGAAGGATGCGGATGCTAATGGCGCTTATTGCATT
GCTCTTAAGGGGCTCTATGAGATAAAGCAGATCACGGAAAACTGGAAAGAAGACGGTAAG
TTTAGTAGAGACAAGCTGAAGATCTCAAATAAAGACTGGTTTGATTTCATACAG. AAC.
AAG.CGG.TAC.CTG
SEQ ID NO: 39
ATGAACAATGGCACTAACAATTTTCAGAATTTCATCGGCATTTCAAGTCTGCAAAAAACT
CTGAGGAATGCTTTGATCCCTACTGAAACCACTCAGCAATTTATAGTCAAGAACGGTATA
ATTAAAGAAGATGAACTCAGGGGTGAAAATAGACAAATACTCAAGGACATTATGGATGAC
TATTATAGAGGCTTCATCTCAGAGACTCTCTCATCAATAGATGATATCGATTGGACTAGC
CTTTTCGAGAAAATGGAGATTCAGTTGAAAAATGGTGATAACAAAGATACGTTGATAAAG
GAACAGACCGAGTACAGGAAAGCCATTCATAAGAAATTTGCTAATGACGATAGATTTAAG
AATATGTTTAGTGCAAAACTGATTAGTGACATTCTGCCGGAGTTCGTTATCCATAATAAT
AACTACTCTGCATCCGAAAAGGAGGAAAAGACGCAAGTTATTAAACTGTTCAGCCGCTTC
GCCACAAGCTTCAAGGACTACTTCAAAAATAGAGCCAACTGCTTTTCTGCCGACGATATA
TCATCATCTTCATGCCATCGGATCGTTAACGATAACGCCGAGATATTCTTCAGCAACGCC
CTTGTATATCGAAGAATAGTCAAAAGTCTGAGTAATGATGATATTAATAAAATTAGCGGT
GATATGAAAGACTCCCTGAAGGAAATGTCACTGGAGGAAATTTATAGTTACGAAAAGTAC
GGCGAATTCATTACTCAAGAAGGCATATCCTTCTATAACGACATTTGCGGAAAGGTCAAC
TCATTCATGAACCTTTATTGCCAGAAGAATAAGGAGAATAAAAATCTTTACAAATTGCAA
AAACTTCACAAACAAATTCTTTGCATCGCGGATACGTCCTACGAAGTTCCTTACAAATTT
GAATCCGATGAGGAAGTGTATCAGAGTGTCAATGGATTTTTGGATAATATCTCTTCAAAA
CATATTGTGGAGAGATTGCGCAAAATAGGTGATAACTACAATGGCTACAACCTGGACAAG
ATTTATATTGTTAGCAAGTTCTATGAAAGTGTCAGTCAAAAGACCTACAGAGATTGGGAG
ACAATCAACACGGCGCTCGAAATACACTACAATAACATCCTCCCCGGCAATGGGAAGAGT
AAAGCCGATAAGGTTAAAAAAGCTGTTAAGAACGACCTCCAGAAATCCATCACGGAAATA
AACGAGCTGGTTTCCAACTATAAGCTGTGTAGCGATGATAATATTAAGGCTGAGACATAT
ATACATGAGATCAGCCACATTCTCAACAATTTCGAGGCACAGGAACTCAAATACAATCCC
GAGATTCACTTGGTGGAAAGTGAGTTGAAGGCGTCAGAGCTTAAGAATGTACTTGACGTA
ATAATGAATGCTTTTCATTGGTGCTCCGTGTTCATGACTGAGGAACTCGTGGATAAGGAT
AATAACTTTTATGCGGAGTTGGAAGAGATATACGATGAAATATACCCGGTTATCTCACTG
TATAATCTGGTCAGAAATTACGTGACCCAAAAGCCTTATAGTACAAAAAAAATAAAGTTG
AACTTCGGTATTCCGACATTGGCAGATGGTTGGTCCAAAAGCAAAGAATACTCTAATAAC
GCCATTATATTGATGCGAGACAATTTGTATTACCTTGGGATCTTTAACGCGAAAAACAAA
CCGGATAAGAAGATCATCGAAGGTAATACATCTGAGAATAAGGGGGATTACAAGAAGATG
ATTTATAATCTGTTGCCGGGGCCAAACAAGATGATTCCGAAGGTCTTTCTGTCATCTAAG
ACAGGAGTAGAGACCTACAAACCTTCTGCGTACATTTTGGAAGGCTACAAACAGAACAAG
CATATAAAATCTAGCAAGGACTTTGATATCACGTTTTGTCATGATCTGATAGATTATTTC
AAAAACTGCATCGCTATACATCCTGAGTGGAAGAATTTCGGCTTTGACTTTTCTGACACC
AGCACATACGAAGACATCTCAGGTTTCTACCGGGAAGTCGAGCTCCAGGGGTACAAGATT
GACTGGACATATATAAGTGAAAAAGACATCGACCTCCTCCAAGAGAAGGGCCAACTTTAC
CTGTTCCAGATCTATAACAAAGACTTTTCTAAAAAGTCCACGGGTAACGACAACTTGCAC
ACTATGTATCTGAAAAACTTGTTCTCTGAAGAGAACCTCAAGGACATCGTCCTGAAGCTT
AACGGGGAGGCGGAGATCTTCTTTAGAAAGTCCTCTATCAAAAATCCCATTATCCATAAA
AAGGGCTCTATACTCGTTAATAGGACATATGAAGCGGAGGAAAAAGATCAATTTGGGAAC
ATCCAGATCGTCCGGAAAAATATACCTGAGAATATCTATCAAGAGCTGTACAAGTATTTT
AATGATAAGTCAGACAAAGAGCTCAGTGATGAGGCGGCAAAGCTCAAGAACGTGGTGGGG
CATCATGAAGCTGCGACGAACATTGTCAAAGATTATAGATACACTTACGATAAATACTTC
CTCCACATGCCGATAACGATTAACTTCAAAGCCAATAAGACGGGGTTTATAAATGATCGG
ATCCTTCAGTACATTGCGAAAGAGAAAGACCTCCATGTGATCGGAATTGACCGAGGAGAA
AGGAATCTGATTTACGTGTCCGTGATTGATACTTGCGGGAATATAGTCGAGCAAAAGAGT
TTCAACATAGTCAACGGGTATGACTATCAGATAAAGCTCAAACAGCAGGAAGGTGCGAGG
CAAATTGCGCGCAAAGAGTGGAAGGAGATAGGCAAGATTAAAGAAATCAAGGAAGGTTAT
CTCAGCTTGGTGATCCATGAAATATCTAAGATGGTTATAAAGTACAATGCCATAATAGCC
ATGGAGGATCTTTCCTACGGGTTTAAGAAGGGCCGATTTAAAGTGGAGCGACAAGTTTAC
CAGAAGTTCGAAACCATGTTGATTAACAAACTTAACTATTTGGTGTTCAAGGATATAAGT
ATAACCGAAAACGGCGGTTTGCTTAAGGGTTATCAGCTCACGTATATTCCTGATAAACTT
AAAAACGTTGGACACCAGTGTGGATGTATCTTCTACGTGCCAGCCGCTTACACTAGTAAG
ATAGATCCTACCACGGGGTTTGTGAATATTTTTAAGTTTAAAGACTTGACAGTCGACGCC
AAAAGGGAATTTATAAAAAAGTTTGATTCTATCCGCTACGATAGTGAAAAAAATCTCTTT
TGCTTTACTTTCGACTATAACAACTTCATTACGCAGAACACTGTCATGAGTAAGTCCAGC
TGGAGCGTCTACACATATGGCGTCCGAATTAAACGACGATTTGTAAACGGGCGGTTTTCA
AACGAATCTGACACGATAGACATTACCAAGGATATGGAGAAGACACTTGAGATGACCGAC
ATAAACTGGGGGACGGTCACGATCTTCGGCAGGACATAATTGATTACGAAATCGTCCAGC
ATATATTCGAAATATTTCGACTTACAGTGCAAATGCGGAACAGTCTCTCTGAACTGGAAG
ATCGCGATTATGACCGGTTGATTTCTCCGGTCCTCAATGAAAATAACATATTTTATGATA
GTGCTAAGGCAGGTGATGCGTTGCCAAAGGATGCAGACGCTAATGGTGCCTATTGTATCG
CGCTCAAGGGATTGTACGAGATAAAGCAAATTACGGAGAACTGGAAGGAGGATGGTAAGT
TTAGCCGAGACAAGTTGAAGATTAGCAATAAAGACTGGTTTGATTTTATCCAAAACAAGA
GGTACCTG
SEQ ID NO: 40
ATGAATAACGGAACTAATAACTTTCAAAATTTCATAGGTATTTCAAGCTTGCAGAAGACC
CTGAGGAATGCCCTGATTCCAACCGAGACAACGCAGCAGTTCATAGTCAAAAATGGCATT
ATTAAGGAAGATGAGCTGCGGGGGGAAAACCGACAGATACTCAAGGATATTATGGACGAC
TATTACCGGGGATTTATCTCAGAAACGCTGAGCAGTATTGATGACATCGATTGGACCAGT
CTTTTCGAGAAAATGGAAATTCAACTTAAGAATGGTGACAATAAAGACACTCTCATAAAG
GAGCAAACTGAATACCGAAAAGCCATACACAAAAAGTTTGCCAACGATGACCGCTTTAAA
AACATGTTTTCAGCTAAGCTCATTAGCGACATTCTCCCCGAGTTTGTGATTCATAACAAT
AACTATAGCGCATCCGAGAAGGAGGAAAAAACCCAAGTTATCAAATTGTTCAGTAGATTC
GCTACGAGCTTTAAAGATTACTTTAAAAACCGGGCTAACTGCTTCAGTGCAGACGATATC
AGCTCCTCATCCTGTCATCGCATCGTCAATGATAATGCTGAGATCTTCTTTTCTAATGCA
CTGGTTTACCGCAGGATAGTTAAGTCTCTTAGTAACGACGACATCAACAAGATATCAGGA
GATATGAAGGATTCCCTTAAAGAAATGAGTCTCGAGGAGATATATTCTTATGAAAAATAC
GGCGAATTTATTACCCAAGAGGGCATTAGTTTCTATAATGACATATGCGGAAAAGTTAAT
AGTTTTATGAATCTCTATTGTCAGAAGAATAAGGAGAATAAGAACCTCTACAAATTGCAG
AAGTTGCACAAGCAAATTCTGTGTATCGCGGACACCTCTTACGAGGTCCCATATAAGTTC
GAGAGTGATGAAGAAGTATACCAGAGCGTTAATGGGTTCCTGGACAACATCTCAAGTAAA
CACATAGTCGAAAGGCTCCGAAAGATCGGTGATAACTATAACGGATATAATTTGGATAAA
ATTTATATAGTTAGCAAATTTTACGAGAGCGTCAGTCAGAAGACCTACCGGGACTGGGAG
ACCATAAACACAGCGCTGGAAATACATTATAACAACATACTGCCTGGGAACGGTAAGTCA
AAGGCAGACAAGGTTAAAAAGGCTGTGAAGAATGACCTGCAAAAATCAATTACAGAAATA
AATGAGTTGGTAAGTAATTACAAACTTTGCAGCGATGATAATATAAAGGCAGAGACGTAC
ATACATGAAATATCTCATATCCTCAACAATTTCGAAGCCCAAGAACTGAAGTACAACCCG
GAAATTCATCTTGTAGAGTCTGAGTTGAAGGCCTCCGAATTGAAAAACGTTCTTGACGTA
ATTATGAATGCCTTCCACTGGTGCTCAGTATTCATGACGGAAGAGCTCGTGGATAAAGAC
AACAATTTTTACGCTGAACTGGAAGAAATATATGACGAGATTTACCCCGTAATTTCACTC
TACAACTTGGTACGAAATTACGTTACCCAAAAGCCATACTCAACAAAAAAAATTAAACTG
AACTTCGGGATACCCACCCTCGCAGATGGATGGTCAAAGTCCAAAGAGTACAGTAACAAT
GCAATTATCCTGATGCGAGACAACCTTTATTACCTCGGGATTTTCAACGCTAAAAATAAA
CCTGATAAAAAAATAATTGAGGGTAATACCTCTGAAAACAAGGGGGATTATAAAAAGATG
ATATACAATCTGCTGCCTGGCCCGAACAAAATGATTCCTAAAGTCTTCTTGTCTTCCAAG
ACTGGAGTCGAAACCTACAAGCCAAGTGCTTATATACTCGAAGGGTACAAACAAAATAAG
CACATAAAATCCAGCAAGGATTTTGATATTACATTCTGCCACGATTTGATTGATTATTTT
AAGAACTGTATAGCCATCCACCCAGAATGGAAGAATTTTGGTTTTGATTTTAGCGATACC
TCAACATATGAGGATATCTCTGGCTTTTACCGCGAGGTAGAACTGCAAGGTTATAAGATC
GATTGGACTTATATTTCTGAAAAGGACATAGATCTCCTGCAAGAGAAAGGGCAACTTTAT
TTGTTTCAAATATACAACAAAGATTTTAGTAAGAAGAGTACTGGCAATGATAACCTTCAC
ACTATGTATCTGAAGAACCTTTTTTCTGAGGAGAACTTGAAGGACATAGTCCTTAAACTC
AATGGGGAAGCTGAAATATTCTTTCGCAAAAGCTCCATTAAAAACCCGATCATTCATAAA
AAGGGTTCCATCTTGGTAAACCGCACATACGAGGCGGAAGAAAAAGATCAGTTCGGAAAT
ATCCAGATCGTAAGGAAGAATATCCCCGAAAATATATACCAAGAGCTTTACAAATATTTT
AACGATAAGTCAGACAAGGAACTGTCAGACGAAGCAGCCAAGTTGAAGAATGTCGTAGGG
CACCACGAAGCAGCTACAAACATAGTTAAAGATTATCGGTACACCTACGATAAATATTTC
CTGCATATGCCAATAACCATAAACTTCAAAGCCAACAAAACAGGGTTCATCAATGACCGA
ATACTTCAGTATATAGCCAAGGAAAAAGACCTGCATGTTATAGGAATAGATAGAGGTGAG
CGCAACTTGATATATGTCAGCGTGATAGACACCTGCGGAAATATCGTCGAGCAAAAAAGT
TTCAACATTGTTAATGGCTACGATTACCAAATTAAATTGAAGCAGCAAGAGGGGGCTCGG
CAAATCGCGCGAAAGGAATGGAAAGAAATCGGGAAGATTAAAGAAATTAAAGAGGGCTAC
CTGTCTCTTGTAATTCACGAAATATCTAAGATGGTCATCAAGTATAATGCCATTATTGCG
ATGGAAGATCTGTCCTACGGATTTAAGAAAGGCAGGTTTAAAGTCGAAAGGCAGGTGTAC
CAGAAATTCGAGACCATGCTGATTAATAAGCTCAACTATCTCGTATTTAAGGATATTTCT
ATAACTGAAAATGGAGGGCTTCTCAAAGGATATCAACTCACATACATACCTGATAAGCTG
AAGAACGTAGGCCACCAGTGTGGATGCATATTCTATGTACCAGCTGCATACACAAGCAAG
ATCGATCCAACTACTGGGTTTGTCAATATCTTCAAATTTAAGGACTTGACGGTCGATGCC
AAACGGGAGTTCATCAAAAAGTTTGATAGTATTCGATATGATAGTGAGAAGAACTTGTTT
TGCTTCACATTTGACTACAACAATTTCATAACGCAAAATACGGTTATGTCTAAATCCTCA
TGGAGCGTCTACACTTACGGAGTGAGGATAAAGCGGCGCTTCGTAAATGGCAGGTTTAGC
AATGAATCCGACACGATTGACATAACCAAGGATATGGAGAAAACCCTCGAGATGACCGAT
ATAAATTGGCGGGATGGACACGATCTGCGACAAGACATAATCGATTATGAAATCGTGCAG
CACATATTTGAGATATTCAGGCTTACGGTCCAAATGAGAAATTCCCTTTCCGAACTTGAA
GACCGCGATTACGACCGACTGATAAGCCCCGTTCTGAACGAAAATAACATCTTCTACGAC
AGCGCTAAAGCGGGAGACGCGCTGCCGAAAGATGCGGACGCAAATGGAGCCTATTGTATC
GCCTTGAAAGGGTTGTACGAGATCAAACAGATAACCGAGAATTGGAAGGAGGATGGGAAG
TTTAGTCGAGACAAACTTAAAATAAGCAACAAGGACTGGTTCGACTTTATTCAAAACAAA
CGATATCTC
SEQ ID NO: 41
ATGAATAATGGTACTAACAATTTTCAAAACTTTATCGGCATCTCTTCACTTCAGAAAACT
CTTCGGAACGCCCTTATACCGACGGAGACAACGCAGCAGTTTATAGTTAAAAACGGGATC
ATTAAAGAAGATGAACTCAGAGGGGAAAACAGGCAAATATTGAAGGACATTATGGACGAT
TACTACCGGGGGTTTATTTCAGAGACCCTTTCATCTATTGATGACATAGATTGGACCTCC
CTTTTCGAGAAAATGGAGATACAATTGAAAAACGGCGACAATAAAGATACACTTATCAAG
GAACAAACTGAGTATCGCAAGGCGATTCACAAGAAGTTTGCGAATGACGATCGCTTTAAG
AATATGTTTTCTGCGAAGCTCATAAGTGACATTCTGCCTGAATTTGTCATTCATAACAAC
AATTATTCTGCTAGCGAAAAAGAGGAAAAAACTCAAGTCATTAAGCTTTTTAGCAGGTTC
GCTACTAGTTTTAAAGACTATTTTAAGAACCGGGCGAATTGCTTTAGCGCTGACGACATA
TCATCCTCATCCTGTCATCGCATAGTCAATGATAATGCAGAAATATTCTTTTCTAATGCG
CTCGTGTATCGGAGAATAGTGAAAAGCCTCTCTAACGATGACATTAACAAAATAAGCGGC
GATATGAAGGATAGTCTGAAGGAAATGTCCCTCGAAGAAATATACTCATACGAGAAGTAC
GGAGAATTTATCACCCAGGAAGGAATTAGTTTTTACAACGACATCTGTGGTAAGGTTAAC
TCTTTTATGAATCTGTATTGTCAAAAGAATAAAGAAAATAAAAATCTTTATAAGCTCCAA
AAGCTTCACAAACAAATCTTGTGCATTGCGGATACGTCATACGAAGTACCTTACAAATTT
GAAAGCGACGAAGAGGTGTATCAGTCAGTGAATGGGTTCCTTGACAATATTTCTAGCAAA
CATATTGTGGAGCGACTTCGAAAGATCGGTGATAATTACAATGGCTATAATTTGGATAAA
ATTTACATAGTTAGTAAGTTTTATGAATCCGTCTCACAAAAGACGTACCGAGATTGGGAG
ACCATCAACACTGCTCTGGAGATTCATTACAATAATATATTGCCTGGGAATGGGAAGTCA
AAGGCCGACAAGGTTAAAAAAGCCGTAAAAAACGATCTTCAAAAGTCCATTACCGAGATA
AATGAACTTGTATCCAACTATAAGTTGTGCTCTGACGATAATATTAAAGCAGAAACGTAT
ATCCACGAAATAAGTCACATCCTGAACAACTTCGAAGCTCAAGAGCTCAAGTATAATCCT
GAAATTCATCTCGTCGAAAGCGAGCTGAAAGCATCCGAGTTGAAGAATGTGCTTGATGTG
ATCATGAACGCATTCCATTGGTGCAGTGTGTTCATGACCGAAGAACTTGTAGACAAAGAC
AACAACTTCTACGCTGAATTGGAAGAGATTTACGATGAAATTTACCCCGTGATATCCCTC
TATAATCTGGTAAGAAATTACGTCACGCAAAAACCATACAGTACCAAGAAAATAAAGCTC
AACTTTGGTATTCCGACGTTGGCAGATGGGTGGAGTAAGAGCAAGGAGTATTCTAACAAT
GCAATCATCCTCATGCGCGACAATTTGTATTATCTGGGGATCTTCAACGCGAAAAATAAG
CCCGACAAAAAGATAATAGAAGGCAATACGTCCGAGAACAAAGGGGACTATAAGAAAATG
ATTTATAACCTTCTTCCAGGACCCAACAAGATGATCCCAAAGGTTTTCTTGAGTTCAAAA
ACCGGCGTAGAAACTTATAAACCGTCCGCCTACATTCTGGAAGGGTACAAGCAAAACAAG
CACATTAAGTCATCTAAGGATTTCGACATTACTTTTTGTCATGATTTGATAGACTACTTC
AAAAATTGTATAGCGATACATCCGGAATGGAAAAATTTTGGGTTCGATTTTTCCGACACA
AGTACTTATGAAGACATCTCAGGGTTTTATAGGGAAGTTGAACTGCAAGGTTACAAAATA
GACTGGACTTATATTAGTGAGAAGGACATTGATTTGCTCCAGGAAAAGGGTCAATTGTAT
CTGTTCCAGATATATAACAAGGATTTCTCTAAAAAATCTACAGGTAACGACAATCTCCAC
ACGATGTACCTCAAGAATCTCTTCAGCGAAGAGAATTTGAAGGATATCGTACTTAAGCTC
AATGGAGAAGCGGAAATATTCTTCAGAAAGTCCAGCATTAAGAATCCTATAATTCACAAG
AAAGGGTCAATTCTCGTAAACCGGACTTATGAGGCCGAAGAAAAAGATCAGTTTGGTAAC
ATTCAGATTGTACGGAAAAACATTCCCGAGAACATCTATCAAGAACTGTATAAATACTTT
AATGATAAATCCGACAAGGAACTTTCTGACGAGGCTGCAAAATTGAAGAACGTAGTGGGA
CACCATGAGGCCGCAACCAATATAGTAAAGGATTACAGATACACTTATGATAAGTATTTC
CTCCATATGCCGATCACGATTAATTTCAAGGCGAATAAAACCGGCTTCATTAACGATCGC
ATTTTGCAATATATTGCGAAGGAAAAGGATTTGCACGTGATAGGTATAGACCGGGGTGAA
CGAAACTTGATTTACGTCTCTGTGATCGACACATGCGGAAATATAGTTGAACAGAAGTCC
TTTAATATTGTGAATGGTTACGACTACCAGATAAAATTGAAGCAACAGGAGGGCGCAAGA
CAGATAGCTCGCAAAGAGTGGAAGGAAATCGGCAAGATCAAAGAAATAAAGGAGGGTTAT
CTTTCCCTGGTAATTCATGAAATTAGCAAGATGGTTATTAAGTATAATGCTATAATAGCT
ATGGAGGACCTTTCCTATGGGTTCAAGAAAGGTCGCTTCAAAGTGGAGCGACAAGTGTAT
CAAAAGTTCGAGACTATGTTGATAAATAAATTGAATTATTTGGTTTTTAAAGACATTTCA
ATAACTGAGAACGGGGGTCTCTTGAAGGGGTACCAATTGACTTATATTCCGGACAAGTTG
AAGAATGTCGGACACCAGTGTGGTTGCATTTTCTACGTGCCTGCCGCTTACACCTCAAAA
ATCGATCCGACCACTGGTTTTGTAAATATATTTAAATTCAAAGATCTCACCGTTGATGCC
AAACGGGAGTTTATCAAAAAATTCGATTCCATTCGCTACGACTCTGAGAAAAACCTTTTT
TGTTTCACGTTCGATTATAACAACTTTATAACCCAAAATACTGTAATGTCCAAGTCAAGT
TGGTCTGTCTATACTTACGGAGTAAGGATCAAGCGCCGCTTCGTTAATGGGAGATTCTCA
AACGAGTCTGATACCATAGACATAACTAAAGACATGGAAAAAACCCTGGAAATGACGGAC
ATCAATTGGCGAGACGGGCATGATCTTCGACAGGACATAATAGATTACGAAATTGTTCAA
CACATTTTCGAGATATTTCGACTTACGGTTCAGATGAGGAATTCCCTTTCCGAATTGGAA
GACCGGGATTATGATCGACTTATATCTCCCGTGCTCAATGAAAACAATATTTTTTATGAT
TCAGCGAAAGCTGGGGACGCGCTGCCAAAAGATGCCGATGCCAATGGAGCATACTGTATC
GCCCTGAAGGGTTTGTATGAGATTAAGCAAATTACTGAAAACTGGAAGGAAGATGGCAAG
TTTTCTAGAGATAAGCTTAAGATTAGCAATAAGGACTGGTTTGACTTCATTCAAAATAAA
AGGTATCTT
SEQ ID NO: 42
ATGAATAATGGAACAAATAATTTTCAAAATTTTATTGGTATCAGTTCATTGCAAAAGACT
TTGAGAAATGCTTTGATCCCGACTGAGACCACACAGCAGTTCATCGTCAAAAATGGCATA
ATCAAGGAAGACGAACTTAGGGGTGAGAATAGACAAATATTGAAGGACATCATGGATGAC
TATTATAGGGGGTTCATTTCCGAAACGCTCAGTAGTATTGATGACATTGACTGGACTAGT
CTTTTCGAGAAAATGGAAATTCAGCTTAAGAACGGGGACAATAAAGACACGCTGATCAAG
GAGCAAACGGAATATAGGAAGGCGATCCATAAAAAATTCGCGAATGATGATCGGTTTAAA
AACATGTTTAGTGCCAAGTTGATCAGCGACATACTGCCCGAATTCGTGATCCACAACAAT
AATTACAGCGCCTCCGAAAAGGAGGAAAAAACTCAGGTCATTAAATTGTTTAGCCGATTC
GCAACGAGTTTCAAAGATTATTTTAAGAACCGGGCCAACTGTTTTTCAGCGGATGATATT
AGCTCCAGCAGCTGCCATCGCATAGTAAATGATAACGCTGAAATCTTTTTTAGCAACGCA
CTTGTCTACCGGAGGATTGTAAAATCACTGTCAAATGATGACATTAACAAAATATCTGGA
GATATGAAGGACTCACTCAAAGAAATGAGCCTGGAAGAAATATATTCATACGAAAAATAC
GGGGAGTTTATTACCCAGGAAGGTATCAGTTTTTATAATGATATATGTGGAAAAGTTAAT
TCATTTATGAATCTTTACTGTCAAAAAAATAAGGAGAACAAGAATTTGTACAAGCTCCAA
AAACTTCATAAACAGATTCTGTGCATCGCAGACACAAGTTATGAGGTACCGTACAAATTT
GAGAGCGACGAAGAAGTTTATCAGAGTGTGAATGGTTTCCTGGACAATATCTCTTCTAAA
CACATTGTTGAGAGGCTTAGGAAGATCGGTGATAATTATAACGGCTATAATCTGGACAAA
ATTTATATTGTATCAAAGTTTTATGAATCAGTCTCTCAAAAGACGTATCGGGATTGGGAA
ACAATTAACACGGCTCTGGAGATCCACTACAATAACATTCTGCCCGGCAACGGGAAGAGC
AAAGCTGATAAGGTCAAGAAGGCAGTCAAGAACGACCTTCAGAAGAGCATAACAGAAATT
AACGAATTGGTCAGTAACTACAAACTGTGTAGTGATGACAACATAAAAGCCGAAACATAC
ATCCATGAAATAAGCCATATCCTGAATAACTTCGAAGCCCAAGAACTTAAATACAATCCC
GAGATTCATCTTGTCGAATCAGAACTCAAGGCGTCCGAGCTCAAAAATGTCCTTGACGTG
ATAATGAATGCCTTCCACTGGTGCAGCGTATTCATGACGGAGGAGTTGGTAGATAAAGAC
AACAACTTTTATGCCGAATTGGAAGAGATTTATGATGAGATTTACCCCGTTATTTCTCTG
TACAACTTGGTTCGAAACTACGTAACACAAAAACCATACTCAACCAAAAAGATCAAACTC
AATTTTGGCATACCTACATTGGCTGATGGTTGGTCCAAGTCAAAGGAATATAGCAATAAT
GCAATAATTCTCATGCGAGATAACTTGTATTATTTGGGGATCTTTAACGCTAAGAACAAA
CCAGATAAAAAGATAATCGAGGGGAACACAAGTGAGAACAAGGGTGATTACAAAAAAATG
ATTTACAATCTGCTTCCTGGGCCTAACAAAATGATTCCGAAGGTGTTTCTTAGCTCTAAA
ACTGGAGTGGAGACGTATAAGCCTTCCGCGTACATTCTCGAAGGCTACAAGCAAAATAAG
CATATCAAGTCCAGTAAGGACTTCGACATCACTTTTTGCCACGATCTCATCGATTACTTT
AAGAACTGTATCGCAATACACCCCGAGTGGAAAAACTTTGGTTTTGATTTTTCAGACACT
AGTACCTACGAGGACATTTCCGGCTTCTATCGAGAAGTCGAACTCCAGGGCTACAAAATC
GATTGGACGTACATTTCTGAGAAGGACATCGACTTGCTCCAAGAGAAAGGTCAACTTTAC
CTCTTCCAAATTTACAATAAAGACTTTTCAAAGAAGAGCACCGGTAATGACAACTTGCAT
ACCATGTATCTGAAGAACCTGTTTTCTGAGGAGAACCTCAAGGATATTGTATTGAAGTTG
AATGGCGAAGCAGAAATATTTTTCCGAAAGTCATCTATCAAGAACCCCATTATACACAAA
AAAGGCTCTATCCTGGTGAACCGGACTTACGAGGCAGAGGAGAAGGATCAATTCGGAAAC
ATACAGATAGTCCGCAAAAACATCCCTGAGAATATCTATCAGGAACTCTATAAGTACTTC
AATGATAAATCAGACAAGGAGCTTAGCGACGAAGCAGCTAAACTTAAAAACGTGGTTGGC
CATCACGAGGCCGCTACCAACATAGTCAAAGACTACCGCTATACTTATGACAAGTACTTT
TTGCACATGCCCATAACAATTAATTTCAAAGCTAACAAAACAGGGTTTATAAATGACAGA
ATCCTCCAATACATCGCCAAAGAGAAGGACCTCCATGTAATCGGGATTGATAGAGGCGAA
CGGAACTTGATTTACGTTAGTGTCATTGATACCTGTGGTAACATTGTCGAACAAAAGTCA
TTCAACATAGTCAATGGATATGATTATCAGATAAAACTCAAGCAACAAGAAGGCGCGAGG
CAGATTGCCAGGAAGGAATGGAAAGAAATCGGGAAGATCAAGGAGATCAAGGAGGGTTAC
CTGTCCTTGGTGATACACGAGATTTCAAAAATGGTTATAAAATACAATGCCATTATCGCG
ATGGAGGATTTGTCTTATGGATTTAAGAAGGGGAGGTTCAAAGTCGAACGACAAGTCTAT
CAGAAGTTTGAAACAATGCTCATTAACAAGCTCAATTACCTTGTTTTCAAGGATATAAGC
ATCACTGAAAACGGCGGACTCCTTAAGGGATATCAGCTGACTTATATCCCCGACAAGCTC
AAGAACGTAGGGCACCAATGCGGATGCATCTTTTACGTGCCTGCAGCATATACTTCAAAA
ATTGATCCGACTACTGGCTTTGTTAACATTTTCAAGTTCAAGGATCTGACGGTAGACGCT
AAGAGAGAATTCATAAAAAAGTTTGACAGCATCAGGTACGATAGTGAAAAGAACCTTTTT
TGTTTTACCTTTGACTACAATAATTTTATTACGCAAAATACAGTTATGAGCAAATCAAGT
TGGAGCGTTTACACATATGGCGTTCGGATCAAGCGCAGATTCGTCAATGGTCGCTTCTCA
AATGAGAGCGATACAATCGATATAACGAAGGATATGGAGAAGACGCTTGAGATGACAGAT
ATCAACTGGCGGGACGGACATGACCTTAGACAAGACATAATCGATTACGAAATAGTACAG
CATATCTTTGAGATTTTTAGGCTTACAGTTCAGATGCGGAACTCTCTTTCCGAACTGGAG
GACCGGGATTATGATCGGTTGATCTCCCCAGTACTGAACGAAAATAATATCTTTTACGAT
AGCGCGAAGGCTGGTGATGCACTCCCAAAAGACGCTGATGCGAACGGAGCTTATTGCATA
GCCCTTAAAGGGCTTTACGAGATTAAACAAATAACAGAAAATTGGAAGGAAGATGGCAAA
TTTTCCCGCGACAAGTTGAAGATTAGTAACAAAGACTGGTTCGACTTCATTCAGAATAAA
CGCTACCTC

Nucleic acid-guided nucleases can encompass a native sequence, an engineered sequence, or engineered nucleotide sequences of synthetized variants. Non-limiting examples of types of engineering that can be done to obtain a non-naturally occurring nuclease system are as follows. Engineering can include codon optimization to facilitate expression or improve expression in a host cell, such as a heterologous host cell. Engineering can reduce the size or molecular weight of the nuclease in order to facilitate expression or delivery. Engineering can alter PAM selection in order to change PAM specificity or to broaden the range of recognized PAMs. Engineering can alter, increase, or decrease stability, processivity, specificity, or efficiency of a targetable nuclease system. Engineering can alter, increase, or decrease protein stability. Engineering can alter, increase, or decrease processivity of nucleic acid scanning. Engineering can alter, increase, or decrease target sequence specificity. Engineering can alter, increase, or decrease nuclease activity. Engineering can alter, increase, or decrease editing efficiency. Engineering can alter, increase, or decrease transformation efficiency. Engineering can alter, increase, or decrease nuclease or guide nucleic acid expression. As used herein, a non-naturally occurring nucleic acid sequence can be an engineered sequence or engineered nucleotide sequences of synthetized variants. Such non-naturally occurring nucleic acid sequences can be amplified, cloned, assembled, synthesized, generated from synthesized oligonucleotides or dNTPs, or otherwise obtained using methods known by those skilled in the art. In certain embodiments, examples of non-naturally occurring nucleic acid-guided nucleases disclosed herein can include those nucleic acid-guided nucleases with engineered polypeptide sequences (e.g., SEQ ID NOs: 2-4) and those nucleotide sequences of synthetized variants (e.g., SEQ ID NOs: 43-63).

SEQ ID NO: 2
MGHHHHHHSSGVDLGTENLYFQSPAAKKKKLDGSVDMNNGTNNFQNFIGISSLQKTLRNA
LIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSA
SEKEEKTQVIKLFSRFATSFKDYFKNRANCFSADDISSSSCHRIVNDNAEIFFSNALVYR
RIVKSLSNDDINKISGDMKDSLKEMSLEEIYSYEKYGEFITQEGISFYNDICGKVNSFMN
LYCQKNKENKNLYKLQKLHKQILCIADTSYEVPYKFESDEEVYQSVNGFLDNISSKHIVE
RLRKIGDNYNGYNLDKIYIVSKFYESVSQKTYRDWETINTALEIHYNNILPGNGKSKADK
VKKAVKNDLQKSITEINELVSNYKLCSDDNIKAETYIHEISHILNNFEAQELKYNPEIHL
VESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKK
IIEGNTSENKGDYKKMIYNLLPGPNKMIPKVFLSSKTGVETYKPSAYILEGYKQNKHIKS
SKDFDITFCHDLIDYFKNCIAIHPEWKNFGFDFSDTSTYEDISGFYREVELQGYKIDWTY
ISEKDIDLLQEKGQLYLFQIYNKDFSKKSTGNDNLHTMYLKNLFSEENLKDIVLKLNGEA
EIFFRKSSIKNPIIHKKGSILVNRTYEAEEKDQFGNIQIVRKNIPENIYQELYKYFNDKS
DKELSDEAAKLKNVVGHHEAATNIVKDYRYTYDKYFLHMPITINFKANKTGFINDRILQY
IAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFE
TMLINKLNYLVFKDISITENGGLLKGYQLTYIPDKLKNVGHQCGCIFYVPAAYTSKIDPT
TGFVNIFKFKDLTVDAKREFIKKFDSIRYDSEKNLFCFTFDYNNFITQNTVMSKSSWSVY
TYGVRIKRRFVNGRFSNESDTIDITKDMEKTLEMTDINWRDGHDLRQDIIDYEIVQHIFE
IFRLTVQMRNSLSELEDRDYDRLISPVLNENNIFYDSAKAGDALPKDADANGAYCIALKG
LYEIKQITENWKEDGKFSRDKLKISNKDWFDFIQNKRYLKRPAATKKAGQAKKKKASGSG
AGSPKKKRKVEDPKKKRKVIPG*
SEQ ID NO: 3
SPAAKKKKLDGSVDMNNGTNNFQNFIGISSLQKTLRNALIPTETTQQFIVKNGIIKEDEL
RGENRQILKDIMDDYYRGFISETLSSIDDIDWTSLFEKMEIQLKNGDNKDTLIKEQTEYR
KAIHKKFANDDRFKNMFSAKLISDILPEFVIHNNNYSASEKEEKTQVIKLFSRFATSFKD
YFKNRANCFSADDISSSSCHRIVNDNAEIFFSNALVYRRIVKSLSNDDINKISGDMKDSL
KEMSLEEIYSYEKYGEFITQEGISFYNDICGKVNSFMNLYCQKNKENKNLYKLQKLHKQI
LCIADTSYEVPYKFESDEEVYQSVNGFLDNISSKHIVERLRKIGDNYNGYNLDKIYIVSK
FYESVSQKTYRDWETINTALEIHYNNILPGNGKSKADKVKKAVKNDLQKSITEINELVSN
YKLCSDDNIKAETYIHEISHILNNFEAQELKYNPEIHLVESELKASELKNVLDVIMNAFH
WCSVFMTEELVDKDNNFYAELEEIYDEIYPVISLYNLVRNYVTQKPYSTKKIKLNFGIPT
LADGWSKSKEYSNNAIILMRDNLYYLGIFNAKNKPDKKIIEGNTSENKGDYKKMIYNLLP
GPNKMIPKVFLSSKTGVETYKPSAYILEGYKQNKHIKSSKDFDITFCHDLIDYFKNCIAI
HPEWKNFGFDFSDTSTYEDISGFYREVELQGYKIDWTYISEKDIDLLQEKGQLYLFQIYN
KDFSKKSTGNDNLHTMYLKNLFSEENLKDIVLKLNGEAEIFFRKSSIKNPIIHKKGSILV
NRTYEAEEKDQFGNIQIVRKNIPENIYQELYKYFNDKSDKELSDEAAKLKNVVGHHEAAT
NIVKDYRYTYDKYFLHMPITINFKANKTGFINDRILQYIAKEKDLHVIGIDRGERNLIYV
SVIDTCGNIVEQKSFNIVNGYDYQIKLKQQEGARQIARKEWKEIGKIKEIKEGYLSLVIH
EISKMVIKYNAIIAMEDLSYGFKKGRFKVERQVYQKFETMLINKLNYLVFKDISITENGG
LLKGYQLTYIPDKLKNVGHQCGCIFYVPAAYTSKIDPTTGFVNIFKFKDLTVDAKREFIK
KFDSIRYDSEKNLFCFTFDYNNFITQNTVMSKSSWSVYTYGVRIKRRFVNGRFSNESDTI
DITKDMEKTLEMTDINWRDGHDLRQDIIDYEIVQHIFEIFRLTVQMRNSLSELEDRDYDR
LISPVLNENNIFYDSAKAGDALPKDADANGAYCIALKGLYEIKQITENWKEDGKFSRDKL
KISNKDWFDFIQNKRYLKRPAATKKAGQAKKKKASGSGAGSPKKKRKVEDPKKKRKVIPG
*
SEQ ID NO: 4
PAAKKKKLDGSVDMNNGTNNFQNFIGISSLQKTLRNALIPTETTQQFIVKNGIIKEDELR
GENRQILKDIMDDYYRGFISETLSSIDDIDWTSLFEKMEIQLKNGDNKDTLIKEQTEYRK
AIHKKFANDDRFKNMFSAKLISDILPEFVIHNNNYSASEKEEKTQVIKLFSRFATSFKDY
FKNRANCFSADDISSSSCHRIVNDNAEIFFSNALVYRRIVKSLSNDDINKISGDMKDSLK
EMSLEEIYSYEKYGEFITQEGISFYNDICGKVNSFMNLYCQKNKENKNLYKLQKLHKQIL
CIADTSYEVPYKFESDEEVYQSVNGFLDNISSKHIVERLRKIGDNYNGYNLDKIYIVSKF
YESVSQKTYRDWETINTALEIHYNNILPGNGKSKADKVKKAVKNDLQKSITEINELVSNY
KLCSDDNIKAETYIHEISHILNNFEAQELKYNPEIHLVESELKASELKNVLDVIMNAFHW
CSVFMTEELVDKDNNFYAELEEIYDEIYPVISLYNLVRNYVTQKPYSTKKIKLNFGIPTL
ADGWSKSKEYSNNAIILMRDNLYYLGIFNAKNKPDKKIIEGNTSENKGDYKKMIYNLLPG
PNKMIPKVFLSSKTGVETYKPSAYILEGYKQNKHIKSSKDFDITFCHDLIDYFKNCIAIH
PEWKNFGFDFSDTSTYEDISGFYREVELQGYKIDWTYISEKDIDLLQEKGQLYLFQIYNK
DFSKKSTGNDNLHTMYLKNLFSEENLKDIVLKLNGEAEIFFRKSSIKNPIIHKKGSILVN
RTYEAEEKDQFGNIQIVRKNIPENIYQELYKYFNDKSDKELSDEAAKLKNVVGHHEAATN
IVKDYRYTYDKYFLHMPITINFKANKTGFINDRILQYIAKEKDLHVIGIDRGERNLIYVS
VIDTCGNIVEQKSFNIVNGYDYQIKLKQQEGARQIARKEWKEIGKIKEIKEGYLSLVIHE
ISKMVIKYNAIIAMEDLSYGFKKGRFKVERQVYQKFETMLINKLNYLVFKDISITENGGL
LKGYQLTYIPDKLKNVGHQCGCIFYVPAAYTSKIDPTTGFVNIFKFKDLTVDAKREFIKK
FDSIRYDSEKNLFCFTFDYNNFITQNTVMSKSSWSVYTYGVRIKRRFVNGRFSNESDTID
ITKDMEKTLEMTDINWRDGHDLRQDIIDYEIVQHIFEIFRLTVQMRNSLSELEDRDYDRL
ISPVLNENNIFYDSAKAGDALPKDADANGAYCIALKGLYEIKQITENWKEDGKFSRDKLK
ISNKDWFDFIQNKRYLKRPAATKKAGQAKKKKASGSGAGSPKKKRKVEDPKKKRKVIPG*
SEQ ID NO: 109:
SMSRRRKANPTKLSENAKKLAKEVENASGSGAGSKRPAATKKAGQAKKKKASGSGAGSPA
AKKKKLDGSVDASGSGAGSPKKKRKVEDASGSGAGSPKKKRKVASGSGAGSMNNGTNNFQ
NFIGISSLQKTLRNALIPTETTQQFIVKNGIIKEDELRGENRQILKDIMDDYYRGFISET
LSSIDDIDWTSLFEKMEIQLKNGDNKDTLIKEQTEYRKAIHKKFANDDRFKNMFSAKLIS
DILPEFVIHNNNYSASEKEEKTQVIKLFSRFATSFKDYFKNRANCFSADDISSSSCHRIV
NDNAEIFFSNALVYRRIVKSLSNDDINKISGDMKDSLKEMSLEEIYSYEKYGEFITQEGI
SFYNDICGKVNSFMNLYCQKNKENKNLYKLQKLHKQILCIADTSYEVPYKFESDEEVYQS
VNGELDNISSKHIVERLRKIGDNYNGYNLDKIYIVSKFYESVSQKTYRDWETINTALEIH
YNNILPGNGKSKADKVKKAVKNDLQKSITEINELVSNYKLCSDDNIKAETYIHEISHILN
NFEAQELKYNPEIHLVESELKASELKNVLDVIMNAFHWCSVFMTEELVDKDNNFYAELEE
IYDEIYPVISLYNLVRNYVTQKPYSTKKIKLNFGIPTLADGWSKSKEYSNNAIILMRDNL
YYLGIFNAKNKPDKKIIEGNTSENKGDYKKMIYNLLPGPNKMIPKVFLSSKTGVETYKPS
AYILEGYKQNKHIKSSKDFDITFCHDLIDYFKNCIAIHPEWKNFGFDFSDTSTYEDISGF
YREVELQGYKIDWTYISEKDIDLLQEKGQLYLFQIYNKDFSKKSTGNDNLHTMYLKNLFS
EENLKDIVLKLNGEAEIFFRKSSIKNPIIHKKGSILVNRTYEAEEKDQFGNIQIVRKNIP
ENIYQELYKYFNDKSDKELSDEAAKLKNVVGHHEAATNIVKDYRYTYDKYFLHMPITINF
KANKTGFINDRILQYIAKEKDLHVIGIDRGERNLIYVSVIDTCGNIVEQKSFNIVNGYDY
QIKLKQQEGARQIARKEWKEIGKIKEIKEGYLSLVIHEISKMVIKYNAIIAMEDLSYGFK
KGRFKVERQVYQKFETMLINKLNYLVFKDISITENGGLLKGYQLTYIPDKLKNVGHQCGC
IFYVPAAYTSKIDPTTGFVNIFKFKDLTVDAKREFIKKFDSIRYDSEKNLFCFTFDYNNF
ITQNTVMSKSSWSVYTYGVRIKRRFVNGRFSNESDTIDITKDMEKTLEMTDINWRDGHDL
RQDIIDYEIVQHIFEIFRLTVQMRNSLSELEDRDYDRLISPVLNENNIFYDSAKAGDALP
KDADANGAYCIALKGLYEIKQITENWKEDGKFSRDKLKISNKDWFDFIQNKRYL
SEQ ID NO: 110:
MSRRRKANPTKLSENAKKLAKEVENASGSGAGSKRPAATKKAGQAKKKKASGSGAGSPAA
KKKKLDGSVDASGSGAGSPKKKRKVEDASGSGAGSPKKKRKVASGSGAGSMNNGTNNFQN
FIGISSLQKTLRNALIPTETTQQFIVKNGIIKEDELRGENRQILKDIMDDYYRGFISETL
SSIDDIDWTSLFEKMEIQLKNGDNKDTLIKEQTEYRKAIHKKFANDDRFKNMFSAKLISD
ILPEFVIHNNNYSASEKEEKTQVIKLFSRFATSFKDYFKNRANCFSADDISSSSCHRIVN
DNAEIFFSNALVYRRIVKSLSNDDINKISGDMKDSLKEMSLEEIYSYEKYGEFITQEGIS
FYNDICGKVNSFMNLYCQKNKENKNLYKLQKLHKQILCIADTSYEVPYKFESDEEVYQSV
NGFLDNISSKHIVERLRKIGDNYNGYNLDKIYIVSKFYESVSQKTYRDWETINTALEIHY
NNILPGNGKSKADKVKKAVKNDLQKSITEINELVSNYKLCSDDNIKAETYIHEISHILNN
FEAQELKYNPEIHLVESELKASELKNVLDVIMNAFHWCSVFMTEELVDKDNNFYAELEEI
YDEIYPVISLYNLVRNYVTQKPYSTKKIKLNFGIPTLADGWSKSKEYSNNAIILMRDNLY
YLGIFNAKNKPDKKIIEGNTSENKGDYKKMIYNLLPGPNKMIPKVFLSSKTGVETYKPSA
YILEGYKQNKHIKSSKDFDITFCHDLIDYFKNCIAIHPEWKNFGFDFSDTSTYEDISGFY
REVELQGYKIDWTYISEKDIDLLQEKGQLYLFQIYNKDFSKKSTGNDNLHTMYLKNLFSE
ENLKDIVLKLNGEAEIFFRKSSIKNPIIHKKGSILVNRTYEAEEKDQFGNIQIVRKNIPE
NIYQELYKYFNDKSDKELSDEAAKLKNVVGHHEAATNIVKDYRYTYDKYFLHMPITINFK
ANKTGFINDRILQYIAKEKDLHVIGIDRGERNLIYVSVIDTCGNIVEQKSFNIVNGYDYQ
IKLKQQEGARQIARKEWKEIGKIKEIKEGYLSLVIHEISKMVIKYNAIIAMEDLSYGFKK
GRFKVERQVYQKFETMLINKLNYLVFKDISITENGGLLKGYQLTYIPDKLKNVGHQCGCI
FYVPAAYTSKIDPTTGFVNIFKFKDLTVDAKREFIKKFDSIRYDSEKNLFCFTFDYNNFI
TQNTVMSKSSWSVYTYGVRIKRRFVNGRFSNESDTIDITKDMEKTLEMTDINWRDGHDLR
QDIIDYEIVQHIFEIFRLTVQMRNSLSELEDRDYDRLISPVLNENNIFYDSAKAGDALPK
DADANGAYCIALKGLYEIKQITENWKEDGKFSRDKLKISNKDWFDFIQNKRYL
SEQ ID NO: 111
GHHHHHHSSGVDLGTENLYFQSMSRRRKANPTKLSENAKKLAKEVENASGSGAGSKRPAA
TKKAGQAKKKKASGSGAGSPAAKKKKLDGSVDASGSGAGSPKKKRKVEDASGSGAGSPKK
KRKVASGSGAGSMNNGTNNFQNFIGISSLQKTLRNALIPTETTQQFIVKNGIIKEDELRG
ENRQILKDIMDDYYRGFISETLSSIDDIDWTSLFEKMEIQLKNGDNKDTLIKEQTEYRKA
IHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKE
MSLEEIYSYEKYGEFITQEGISFYNDICGKVNSFMNLYCQKNKENKNLYKLQKLHKQILC
IADTSYEVPYKFESDEEVYQSVNGFLDNISSKHIVERLRKIGDNYNGYNLDKIYIVSKFY
ESVSQKTYRDWETINTALEIHYNNILPGNGKSKADKVKKAVKNDLQKSITEINELVSNYK
LCSDDNIKAETYIHEISHILNNFEAQELKYNPEIHLVESELKASELKNVLDVIMNAFHWC
SVFMTEELVDKDNNFYAELEEIYDEIYPVISLYNLVRNYVTQKPYSTKKIKLNFGIPTLA
DGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHP
EWKNFGFDFSDTSTYEDISGFYREVELQGYKIDWTYISEKDIDLLQEKGQLYLFQIYNKD
FSKKSTGNDNLHTMYLKNLFSEENLKDIVLKLNGEAEIFFRKSSIKNPIIHKKGSILVNR
TYEAEEKDQFGNIQIVRKNIPENIYQELYKYFNDKSDKELSDEAAKLKNVVGHHEAATNI
VKDYRYTYDKYFLHMPITINFKANKTGFINDRILQYIAKEKDLHVIGIDRGERNLIYVSV
IDTCGNIVEQKSFNIVNGYDYQIKLKQQEGARQIARKEWKEIGKIKEIKEGYLSLVIHEI
SKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKF
DSIRYDSEKNLFCFTFDYNNFITQNTVMSKSSWSVYTYGVRIKRRFVNGRFSNESDTIDI
TKDMEKTLEMTDINWRDGHDLRQDIIDYEIVQHIFEIFRLTVQMRNSLSELEDRDYDRLI
SPVLNENNIFYDSAKAGDALPKDADANGAYCIALKGLYEIKQITENWKEDGKFSRDKLKI
SNKDWFDFIQNKRYL*
SEQ ID NO: 112
MGHHHHHHSSGVDLGTENLYFQSMSRRRKANPTKLSENAKKLAKEVENASGSGAGSKRPA
ATKKAGQAKKKKASGSGAGSPAAKKKKLDGSVDASGSGAGSPKKKRKVEDASGSGAGSPK
KKRKVASGSGAGSMNNGTNNFQNFIGISSLQKTLRNALIPTETTQQFIVKNGIIKEDELR
GENRQILKDIMDDYYRGFISETLSSIDDIDWTSLFEKMEIQLKNGDNKDTLIKEQTEYRK
AIHKKFANDDRFKNMFSAKLISDILPEFVIHNNNYSASEKEEKTQVIKLFSRFATSFKDY
FKNRANCFSADDISSSSCHRIVNDNAEIFFSNALVYRRIVKSLSNDDINKISGDMKDSLK
EMSLEEIYSYEKYGEFITQEGISFYNDICGKVNSFMNLYCQKNKENKNLYKLQKLHKQIL
CIADTSYEVPYKFESDEEVYQSVNGFLDNISSKHIVERLRKIGDNYNGYNLDKIYIVSKF
YESVSQKTYRDWETINTALEIHYNNILPGNGKSKADKVKKAVKNDLQKSITEINELVSNY
KLCSDDNIKAETYIHEISHILNNFEAQELKYNPEIHLVESELKASELKNVLDVIMNAFHW
CSVFMTEELVDKDNNFYAELEEIYDEIYPVISLYNLVRNYVTQKPYSTKKIKLNFGIPTL
ADGWSKSKEYSNNAIILMRDNLYYLGIFNAKNKPDKKIIEGNTSENKGDYKKMIYNLLPG
PNKMIPKVFLSSKTGVETYKPSAYILEGYKQNKHIKSSKDFDITFCHDLIDYFKNCIAIH
PEWKNFGFDFSDTSTYEDISGFYREVELQGYKIDWTYISEKDIDLLQEKGQLYLFQIYNK
DFSKKSTGNDNLHTMYLKNLFSEENLKDIVLKLNGEAEIFFRKSSIKNPIIHKKGSILVN
RTYEAEEKDQFGNIQIVRKNIPENIYQELYKYFNDKSDKELSDEAAKLKNVVGHHEAATN
IVKDYRYTYDKYFLHMPITINFKANKTGFINDRILQYIAKEKDLHVIGIDRGERNLIYVS
VIDTCGNIVEQKSFNIVNGYDYQIKLKQQEGARQIARKEWKEIGKIKEIKEGYLSLVIHE
ISKMVIKYNAIIAMEDLSYGFKKGRFKVERQVYQKFETMLINKLNYLVFKDISITENGGL
LKGYQLTYIPDKLKNVGHQCGCIFYVPAAYTSKIDPTTGFVNIFKFKDLTVDAKREFIKK
FDSIRYDSEKNLFCFTFDYNNFITQNTVMSKSSWSVYTYGVRIKRRFVNGRFSNESDTID
ITKDMEKTLEMTDINWRDGHDLRQDIIDYEIVQHIFEIFRLTVQMRNSLSELEDRDYDRL
ISPVLNENNIFYDSAKAGDALPKDADANGAYCIALKGLYEIKQITENWKEDGKFSRDKLK
ISNKDWFDFIQNKRYL*
SEQ ID NO: 43
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAACGGT
ACCAATAACTTCCAGAACTTCATCGGTATTTCTAGCCTGCAAAAGACCCTGCGTAACGCG
CTGATTCCGACCGAGACTACCCAGCAATTCATCGTGAAAAACGGTATCATTAAGGAAGAT
GAATTGCGCGGTGAGAATCGTCAGATTCTGAAAGATATCATGGATGACTACTATCGCGGT
TTCATTAGCGAAACCCTGTCGAGCATCGATGATATCGATTGGACGAGCCTCTTCGAGAAA
ATGGAAATTCAACTGAAAAATGGTGACAACAAAGATACCCTGATTAAAGAACAAACGGAA
TACCGCAAGGCAATCCATAAAAAGTTTGCGAATGACGACCGTTTTAAGAATATGTTCTCG
GCCAAGCTGATTTCCGACATCCTGCCAGAGTTCGTCATTCACAACAACAATTACAGCGCA
AGCGAGAAAGAGGAAAAGACTCAGGTCATTAAGCTGTTTAGCCGCTTTGCGACGTCCTTC
AAAGACTACTTCAAGAATCGTGCGAATTGCTTTAGCGCGGATGACATCTCTAGCTCTAGC
TGTCACCGTATTGTTAACGACAATGCAGAGATTTTCTTCAGCAACGCCCTGGTGTATCGC
CGTATTGTCAAGTCTCTGAGCAACGACGACATTAACAAGATCAGCGGCGACATGAAAGAC
AGCCTGAAAGAAATGTCTCTGGAAGAAATCTACAGCTACGAGAAATATGGTGAGTTTATC
ACCCAAGAGGGCATTAGCTTCTACAATGATATCTGTGGTAAGGTTAATAGCTTTATGAAT
CTGTACTGCCAGAAGAATAAAGAAAACAAGAACTTGTACAAGCTGCAAAAGCTGCATAAG
CAAATTCTGTGCATCGCCGATACTAGCTATGAAGTTCCGTACAAGTTCGAGTCTGATGAA
GAGGTGTATCAGTCAGTCAACGGTTTTCTGGATAACATCAGCAGCAAGCACATCGTCGAG
CGCCTGCGCAAGATTGGTGACAACTACAATGGTTATAACCTGGACAAGATCTATATCGTG
TCGAAGTTTTACGAGAGCGTGTCCCAGAAAACGTACCGTGATTGGGAAACGATTAACACG
GCCTTGGAAATTCACTATAACAATATCCTGCCGGGCAACGGCAAGAGCAAAGCTGACAAA
GTCAAAAAAGCTGTGAAAAACGATCTGCAAAAGTCCATCACCGAGATCAACGAACTGGTT
AGCAACTATAAGCTGTGTAGCGACGACAACATTAAAGCTGAAACGTATATCCACGAAATC
AGCCACATCCTGAATAACTTTGAGGCACAAGAACTGAAATACAATCCTGAGATCCATCTG
GTAGAGAGCGAGCTGAAGGCAAGCGAGTTGAAAAACGTTCTCGACGTTATCATGAATGCT
TTCCACTGGTGTAGCGTGTTTATGACCGAAGAACTGGTTGACAAAGATAACAATTTCTAT
GCAGAGCTGGAAGAAATCTATGATGAAATCTACCCGGTCATCAGCCTGTATAACCTGGTT
CGTAACTACGTGACGCAGAAGCCGTACAGCACCAAAAAGATCAAGCTGAACTTCGGTATT
CCGACCTTGGCGGACGGTTGGAGCAAATCCAAAGAATACTCCAATAATGCGATTATTCTG
ATGCGTGATAATCTGTACTATCTGGGTATCTTCAATGCGAAGAACAAGCCAGATAAAAAG
ATTATTGAAGGCAACACCAGCGAGAATAAAGGCGACTACAAGAAAATGATCTACAACTTA
TTGCCGGGTCCGAACAAGATGATCCCGAAAGTTTTTCTGAGCAGCAAGACCGGCGTTGAA
ACCTATAAGCCGAGCGCGTACATTTTAGAGGGCTATAAACAAAACAAGCACATCAAGAGC
AGCAAAGATTTTGATATTACGTTCTGCCACGACCTGATCGACTATTTCAAGAATTGTATT
GCGATTCACCCTGAGTGGAAGAACTTCGGTTTTGACTTTTCCGATACCTCCACCTATGAA
GATATTAGCGGTTTTTACCGTGAAGTCGAGTTGCAGGGTTATAAGATTGATTGGACTTAC
ATTTCCGAGAAAGACATCGACCTGTTGCAAGAGAAAGGTCAGCTGTACCTGTTTCAGATC
TATAACAAAGATTTCAGCAAAAAGTCGACGGGCAATGATAATCTGCACACCATGTATCTG
AAAAACCTGTTTAGCGAAGAGAACCTGAAAGACATTGTTCTTAAGCTGAATGGTGAGGCC
GAGATCTTCTTCCGTAAAAGCTCCATTAAGAACCCGATTATCCACAAAAAGGGCTCTATT
CTGGTTAACCGCACGTACGAAGCGGAAGAGAAAGATCAATTTGGTAACATCCAGATCGTG
CGTAAGAATATCCCGGAGAACATTTACCAAGAACTGTATAAGTATTTCAATGACAAGAGC
GATAAAGAATTGAGCGATGAAGCGGCAAAGCTGAAAAACGTCGTTGGCCACCACGAAGCC
GCGACGAATATCGTGAAAGATTATCGTTACACCTACGACAAGTACTTTCTGCACATGCCG
ATCACCATCAATTTCAAAGCGAATAAAACGGGTTTTATCAATGACCGTATCCTGCAGTAC
ATTGCGAAAGAAAAAGATTTACACGTGATTGGTATTGATCGCGGCGAGCGCAATCTGATT
TACGTCAGCGTTATCGACACGTGCGGCAATATTGTGGAGCAGAAAAGCTTCAATATCGTC
AATGGTTACGACTACCAGATCAAACTGAAGCAACAAGAGGGCGCCCGCCAGATTGCGCGT
AAAGAGTGGAAAGAAATCGGTAAGATTAAAGAAATCAAGGAAGGCTACCTGTCCCTGGTG
ATCCATGAAATCAGCAAAATGGTGATCAAGTACAACGCTATCATTGCGATGGAAGATCTG
AGCTACGGTTTTAAAAAGGGTCGCTTCAAAGTTGAGCGTCAAGTGTATCAGAAATTTGAG
ACTATGCTGATTAACAAGTTGAACTATCTGGTTTTTAAAGACATCAGCATTACCGAGAAT
GGTGGCCTGCTGAAGGGTTATCAACTGACCTATATTCCTGACAAGTTGAAAAATGTTGGT
CATCAGTGTGGTTGCATTTTCTACGTACCGGCAGCGTACACGAGCAAGATTGACCCGACC
ACGGGTTTCGTTAACATTTTCAAGTTTAAAGATTTGACCGTGGACGCCAAGCGTGAGTTC
ATTAAAAAGTTCGACAGCATCAGATACGACTCTGAGAAGAATCTGTTCTGCTTTACGTTC
GACTACAATAACTTCATTACCCAAAATACCGTTATGAGCAAAAGCTCCTGGAGCGTGTAC
ACGTACGGCGTCCGTATCAAGCGTCGTTTTGTGAATGGTCGCTTTTCCAACGAATCTGAC
ACCATTGACATTACCAAAGATATGGAAAAGACCCTTGAGATGACCGACATTAATTGGCGT
GATGGCCATGACTTGCGCCAAGACATTATCGACTACGAAATTGTTCAGCACATCTTTGAG
ATTTTTCGTCTGACGGTCCAGATGCGCAACTCGCTGAGCGAGTTGGAAGATCGTGACTAT
GACCGTCTGATTAGCCCGGTGCTGAATGAAAACAATATCTTCTATGATAGCGCAAAGGCC
GGTGACGCGCTGCCGAAAGATGCGGATGCTAACGGTGCATACTGCATTGCACTGAAGGGT
CTGTACGAAATCAAACAGATCACCGAGAATTGGAAAGAGGATGGTAAGTTTAGCCGTGAT
AAGCTGAAGATTAGCAATAAAGACTGGTTCGACTTTATTCAAAACAAGCGCTATCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 44
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGA
ACAAATAATTTTCAGAACTTTATTGGGATCAGTTCGCTTCAGAAAACGCTTCGTAATGCT
CTGATTCCCACAGAAACCACTCAGCAGTTTATCGTAAAGAATGGCATTATCAAGGAGGAT
GAATTACGCGGCGAGAACCGCCAAATCTTAAAAGATATCATGGACGACTACTACCGCGGT
TTCATTAGCGAAACTCTTAGTTCAATTGACGACATTGACTGGACGTCCTTGTTCGAAAAG
ATGGAGATTCAATTAAAGAACGGTGATAACAAGGATACGTTGATTAAAGAACAGACGGAG
TACCGTAAGGCTATCCACAAAAAATTTGCAAACGACGACCGCTTTAAAAATATGTTTAGC
GCAAAATTAATCTCCGACATCCTGCCTGAATTCGTCATCCATAACAATAACTATAGCGCC
TCGGAAAAAGAAGAAAAAACGCAGGTTATTAAACTTTTCTCGCGCTTTGCAACAAGCTTT
AAGGATTACTTCAAAAATCGCGCCAATTGTTTTTCAGCCGACGACATTAGCTCCAGTTCC
TGCCACCGTATTGTGAATGACAACGCTGAGATTTTTTTTTCCAATGCGCTGGTTTATCGT
CGTATTGTTAAGAGCCTTAGTAACGACGACATTAATAAAATTAGCGGTGATATGAAGGAT
AGCTTGAAAGAAATGAGTCTGGAAGAGATCTATAGTTACGAGAAGTACGGCGAATTTATT
ACCCAGGAGGGCATTTCATTTTACAATGATATCTGTGGAAAAGTCAACTCCTTTATGAAC
TTGTATTGCCAAAAGAATAAAGAAAACAAAAACCTGTACAAACTGCAAAAGTTACACAAG
CAGATTTTGTGTATCGCAGACACGTCATACGAAGTACCGTACAAGTTTGAGTCCGATGAA
GAAGTGTACCAAAGCGTTAATGGCTTTTTGGATAACATTTCGAGCAAACATATCGTAGAG
CGTTTGCGTAAGATTGGTGATAATTACAACGGTTACAATTTAGACAAAATCTATATCGTC
TCTAAGTTTTACGAAAGTGTTTCTCAGAAAACTTACCGCGATTGGGAGACGATCAACACT
GCGCTGGAGATTCATTACAATAATATCCTTCCAGGTAACGGTAAAAGCAAAGCTGATAAG
GTGAAAAAGGCGGTTAAAAATGACCTTCAAAAGTCTATCACAGAAATCAACGAATTGGTC
AGCAATTATAAGCTTTGCAGTGACGATAACATTAAGGCCGAGACTTACATCCATGAGATC
TCTCACATTCTTAATAATTTTGAAGCGCAAGAGCTGAAATACAATCCTGAAATCCATCTG
GTCGAAAGTGAATTAAAAGCCTCCGAATTAAAAAATGTCTTGGACGTGATCATGAATGCG
TTCCATTGGTGCTCAGTTTTTATGACGGAAGAGTTGGTGGACAAAGACAACAATTTTTAC
GCCGAGCTTGAGGAAATTTACGACGAAATTTACCCCGTTATTTCGTTATACAACCTTGTG
CGTAATTACGTTACACAAAAGCCCTATTCGACAAAGAAAATCAAGTTAAATTTCGGGATT
CCCACATTAGCTGATGGATGGTCCAAATCCAAAGAATACTCGAATAACGCTATCATCCTT
ATGCGTGATAATTTGTACTACTTAGGCATCTTCAATGCGAAGAACAAACCTGACAAGAAA
ATTATCGAAGGAAACACTTCGGAGAACAAAGGTGATTATAAAAAGATGATCTACAACTTG
CTTCCCGGGCCAAACAAAATGATTCCCAAGGTATTTTTGAGTTCTAAAACCGGTGTCGAA
ACTTACAAACCAAGTGCTTATATTTTGGAAGGATACAAACAGAACAAACATATCAAGTCT
TCGAAAGACTTCGATATTACGTTCTGCCACGATCTGATCGATTACTTCAAGAACTGTATT
GCTATTCACCCCGAGTGGAAGAACTTTGGATTTGATTTCTCCGACACGTCCACTTATGAA
GATATCTCTGGCTTCTATCGCGAGGTTGAATTACAAGGGTATAAGATTGACTGGACTTAT
ATTTCGGAGAAGGATATCGATCTTTTGCAAGAAAAAGGGCAACTTTATTTATTTCAGATC
TATAACAAGGACTTTTCAAAAAAGAGCACTGGAAATGACAATCTGCATACCATGTACCTT
AAGAACCTGTTCTCGGAAGAGAACCTGAAGGACATTGTACTTAAACTGAATGGAGAGGCA
GAGATCTTCTTTCGCAAATCAAGCATTAAGAACCCAATTATTCACAAAAAGGGGAGTATC
TTAGTAAATCGCACATATGAGGCTGAGGAAAAAGATCAGTTTGGTAACATTCAGATCGTG
CGTAAGAACATTCCTGAAAATATCTATCAGGAACTTTATAAGTATTTCAACGATAAAAGT
GATAAAGAGCTGAGTGACGAAGCGGCTAAACTTAAGAATGTTGTGGGACACCATGAGGCA
GCAACCAATATTGTGAAGGATTATCGCTATACGTACGACAAATACTTTTTACACATGCCC
ATCACTATTAATTTTAAAGCTAATAAGACTGGCTTCATTAACGATCGCATCCTGCAGTAC
ATTGCTAAGGAAAAGGATCTTCACGTTATCGGTATCGATCGCGGGGAGCGTAATCTTATC
TACGTCTCTGTCATTGACACGTGTGGCAATATTGTGGAGCAAAAGTCCTTCAATATTGTT
AACGGCTATGACTATCAGATTAAATTGAAACAGCAGGAAGGTGCGCGTCAGATTGCCCGC
AAGGAATGGAAGGAAATTGGCAAGATCAAAGAAATTAAGGAGGGCTACTTAAGCTTAGTA
ATTCACGAAATTAGTAAAATGGTTATCAAATACAACGCCATCATCGCGATGGAGGATCTT
TCGTACGGGTTTAAGAAAGGTCGTTTTAAAGTGGAGCGTCAGGTGTACCAGAAATTTGAA
ACTATGCTTATTAACAAACTTAACTACCTGGTTTTCAAGGATATCAGTATTACTGAAAAC
GGGGGGCTGTTAAAAGGGTATCAATTAACTTACATTCCAGACAAATTAAAGAACGTTGGA
CATCAGTGTGGCTGCATTTTTTATGTACCAGCTGCATACACTTCAAAGATCGATCCTACG
ACTGGGTTCGTGAACATTTTTAAGTTTAAAGACTTGACGGTAGATGCCAAGCGCGAATTC
ATCAAGAAATTCGACAGCATTCGCTACGACTCTGAGAAAAATCTTTTCTGTTTCACATTC
GATTATAACAATTTCATTACGCAGAACACAGTAATGTCCAAGTCTTCTTGGAGTGTTTAT
ACATATGGTGTCCGCATTAAGCGCCGTTTCGTCAACGGCCGCTTCAGTAATGAGAGCGAT
ACTATTGACATCACAAAAGACATGGAAAAAACACTGGAAATGACCGACATCAATTGGCGT
GACGGCCATGACTTACGTCAGGATATCATTGATTATGAGATCGTTCAACACATCTTCGAA
ATCTTTCGCTTGACTGTTCAAATGCGCAATTCCTTGTCGGAATTGGAGGACCGTGATTAT
GACCGCTTAATTTCCCCCGTCTTAAATGAAAACAATATTTTTTATGACTCTGCAAAAGCT
GGAGATGCTCTGCCGAAAGACGCCGATGCAAATGGGGCATATTGCATTGCTTTAAAGGGG
CTTTACGAGATCAAGCAAATCACCGAAAACTGGAAAGAGGATGGAAAGTTTTCGCGTGAT
AAACTGAAGATCTCTAACAAAGACTGGTTCGACTTTATCCAGAACAAGCGTTATTTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 45
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGC
ACCAATAACTTCCAAAACTTCATCGGGATCTCTAGCCTTCAGAAGACGCTTCGCAATGCT
CTTATCCCAACTGAGACCACTCAACAATTTATTGTGAAGAATGGAATTATTAAAGAGGAC
GAACTGCGTGGCGAGAATCGTCAGATCTTAAAGGACATTATGGATGATTATTACCGTGGA
TTCATCTCCGAAACATTATCGTCGATCGATGATATCGATTGGACTTCTCTGTTCGAGAAA
ATGGAAATTCAATTGAAAAACGGAGATAATAAAGATACGCTTATCAAAGAACAGACGGAA
TATCGTAAAGCGATTCATAAGAAATTCGCAAATGACGATCGTTTCAAAAATATGTTCAGT
GCCAAGCTTATTTCGGACATTTTACCTGAATTTGTAATTCATAATAATAACTACTCAGCA
AGTGAGAAGGAGGAGAAAACCCAAGTTATTAAACTGTTCTCTCGTTTCGCAACGTCCTTT
AAAGATTACTTTAAAAACCGCGCGAATTGCTTTAGCGCTGACGACATTTCCAGCTCATCC
TGTCATCGCATCGTAAACGACAATGCGGAAATCTTCTTCAGCAACGCCCTGGTTTACCGC
CGCATCGTCAAAAGCTTATCGAATGACGACATCAATAAGATCTCAGGAGATATGAAGGAC
TCGCTTAAGGAGATGTCTCTGGAGGAAATTTATAGTTACGAAAAGTATGGAGAGTTCATT
ACCCAGGAGGGAATCTCGTTCTACAATGACATTTGCGGGAAGGTGAACTCCTTCATGAAC
TTATACTGCCAGAAAAACAAAGAGAACAAAAATCTGTATAAATTGCAGAAATTACATAAA
CAGATTCTTTGTATTGCTGACACTTCCTACGAAGTACCCTATAAATTCGAGTCAGATGAA
GAAGTATACCAGTCCGTGAACGGATTTCTGGACAATATCTCCTCAAAACACATCGTGGAA
CGCTTACGTAAAATTGGCGATAATTATAATGGTTACAATCTTGACAAAATTTATATCGTA
TCTAAATTTTACGAGAGTGTGAGCCAAAAGACCTACCGCGACTGGGAGACCATCAACACA
GCTTTAGAAATTCACTATAATAATATCTTACCCGGCAATGGTAAGAGCAAGGCTGACAAG
GTAAAAAAGGCCGTCAAGAATGATTTGCAGAAATCTATTACAGAAATTAATGAGTTAGTC
TCCAACTATAAGCTTTGTTCCGACGATAACATCAAAGCTGAGACATATATTCATGAGATT
AGTCACATTCTTAACAACTTCGAGGCCCAGGAACTTAAGTACAATCCTGAAATTCATCTT
GTCGAGTCTGAGCTGAAAGCTAGTGAATTGAAAAATGTTTTAGACGTTATTATGAACGCA
TTCCACTGGTGCTCTGTGTTTATGACAGAAGAACTGGTCGACAAGGACAATAACTTCTAT
GCCGAACTTGAGGAAATCTACGATGAAATTTACCCTGTAATCTCCTTGTATAATCTTGTA
CGTAATTACGTCACTCAAAAACCTTACAGCACGAAAAAAATTAAATTGAACTTCGGGATT
CCTACACTTGCCGACGGGTGGTCTAAATCCAAGGAATATAGCAACAATGCCATTATTTTA
ATGCGCGACAATCTTTACTATTTAGGAATTTTTAACGCTAAGAACAAGCCCGATAAAAAG
ATTATTGAAGGAAACACGTCTGAAAATAAGGGCGACTACAAAAAGATGATTTATAACCTT
TTGCCCGGTCCAAACAAAATGATCCCAAAGGTATTCCTGTCATCCAAAACAGGGGTTGAG
ACATATAAGCCCAGCGCATATATTCTGGAAGGATACAAACAGAATAAACATATCAAAAGC
AGCAAAGATTTTGACATTACTTTTTGCCACGATTTAATCGACTACTTCAAAAACTGTATC
GCTATCCACCCTGAATGGAAGAATTTCGGATTTGATTTCTCAGATACAAGTACGTATGAG
GATATCAGCGGTTTCTATCGCGAAGTTGAACTTCAAGGGTATAAAATTGACTGGACCTAC
ATTAGTGAGAAGGACATCGACCTGTTACAGGAAAAAGGCCAATTGTACTTGTTTCAGATC
TACAATAAGGATTTCTCAAAAAAATCGACCGGCAATGATAACTTGCACACCATGTACCTG
AAGAACCTTTTTTCGGAGGAAAACCTTAAAGACATTGTCCTGAAGTTGAATGGAGAAGCG
GAGATTTTCTTTCGTAAGTCTTCCATTAAAAATCCAATTATTCATAAGAAGGGCAGCATC
CTTGTGAACCGTACGTACGAGGCGGAAGAGAAGGACCAATTCGGTAACATTCAAATCGTC
CGCAAGAACATCCCTGAAAATATTTATCAGGAGCTTTACAAGTATTTCAATGATAAGTCC
GACAAGGAATTATCAGATGAGGCTGCGAAGTTGAAAAATGTTGTTGGTCATCACGAGGCG
GCGACGAATATTGTAAAGGATTATCGCTACACTTATGACAAGTACTTTCTGCACATGCCG
ATCACCATTAATTTCAAGGCGAACAAAACAGGATTTATTAATGACCGCATCTTACAATAC
ATTGCCAAAGAAAAGGACTTACACGTTATTGGCATTGATCGTGGAGAACGCAACTTAATC
TACGTAAGCGTTATTGACACTTGCGGGAATATCGTAGAACAAAAGAGCTTCAACATCGTG
AATGGTTACGATTACCAGATCAAGCTTAAGCAGCAGGAGGGAGCGCGCCAGATCGCGCGC
AAGGAATGGAAGGAGATTGGTAAGATCAAGGAAATCAAGGAAGGTTATCTGTCCTTGGTA
ATCCACGAAATTTCGAAAATGGTTATCAAATACAATGCTATTATTGCAATGGAGGACTTG
TCCTACGGCTTTAAAAAAGGACGCTTTAAGGTGGAGCGCCAGGTTTATCAAAAGTTTGAA
ACAATGCTGATTAACAAGCTGAACTATTTGGTCTTTAAAGATATCTCCATCACCGAAAAT
GGTGGGCTTTTGAAAGGCTATCAACTTACATATATCCCTGATAAGCTTAAGAATGTGGGT
CATCAGTGCGGGTGCATTTTTTATGTTCCTGCAGCCTACACGTCCAAAATCGATCCTACA
ACTGGATTTGTTAATATCTTCAAATTTAAGGATCTTACCGTCGACGCGAAGCGCGAATTT
ATCAAGAAATTCGATAGTATTCGTTATGATTCCGAAAAAAACCTTTTCTGTTTCACCTTT
GATTATAATAACTTTATCACGCAAAATACTGTCATGAGCAAATCGAGTTGGTCTGTGTAC
ACTTACGGAGTACGCATCAAGCGTCGTTTTGTTAATGGGCGCTTCAGTAACGAGTCAGAC
ACGATTGATATCACAAAAGATATGGAGAAAACGCTGGAGATGACAGACATCAATTGGCGC
GATGGTCATGACTTACGTCAAGACATTATCGATTATGAAATTGTCCAGCATATCTTTGAG
ATCTTTCGTTTGACTGTTCAGATGCGCAACAGCCTGTCAGAATTGGAGGATCGTGACTAT
GATCGCCTTATTTCTCCCGTCTTAAATGAGAACAATATCTTCTACGACTCAGCCAAGGCT
GGAGATGCACTGCCAAAAGACGCCGACGCAAATGGGGCCTACTGTATTGCATTGAAGGGG
TTGTACGAGATCAAACAGATTACAGAAAATTGGAAGGAGGACGGTAAGTTCTCTCGTGAT
AAGCTGAAGATTTCTAACAAAGACTGGTTCGATTTCATTCAGAACAAACGTTACCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 46
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGT
ACCAATAACTTTCAGAATTTCATTGGAATCAGCAGCTTACAGAAAACCCTGCGCAATGCA
CTTATCCCCACTGAGACAACCCAGCAGTTCATTGTAAAGAACGGGATTATTAAAGAAGAT
GAGCTTCGCGGGGAGAATCGTCAGATCTTAAAGGATATTATGGACGATTACTACCGTGGC
TTCATTTCGGAGACGCTGTCGTCGATCGACGACATCGACTGGACATCCTTGTTTGAAAAG
ATGGAAATCCAACTGAAGAATGGCGATAACAAGGACACGTTAATCAAAGAGCAGACGGAA
TACCGTAAAGCTATCCACAAAAAGTTCGCTAATGACGACCGCTTTAAGAACATGTTCTCA
GCAAAACTTATTAGCGATATTTTACCTGAATTTGTCATCCACAATAACAATTACTCCGCG
AGTGAAAAAGAGGAGAAAACCCAGGTGATTAAGCTGTTTTCCCGTTTTGCAACCAGTTTC
AAGGACTATTTTAAGAATCGTGCTAATTGTTTCTCTGCAGACGACATTTCCTCGTCGTCC
TGCCATCGCATTGTTAATGATAATGCTGAAATCTTTTTTTCAAACGCACTTGTGTATCGT
CGCATTGTCAAAAGCTTAAGTAATGACGATATCAATAAGATCTCAGGAGACATGAAGGAC
TCCCTGAAAGAAATGTCATTGGAAGAAATTTACTCTTATGAAAAGTATGGAGAATTTATT
ACGCAGGAGGGTATCAGCTTCTATAACGACATTTGTGGTAAAGTGAACAGCTTTATGAAT
CTTTATTGTCAAAAGAATAAAGAGAACAAAAATCTGTACAAGCTGCAGAAATTGCATAAA
CAAATTCTGTGCATTGCAGATACTTCGTATGAGGTTCCTTACAAATTCGAGTCGGATGAG
GAGGTGTATCAAAGCGTAAACGGATTTTTGGATAACATTAGTAGTAAGCATATTGTGGAA
CGCCTTCGCAAGATTGGTGACAACTATAACGGATACAACTTAGACAAGATCTATATTGTC
TCGAAGTTTTACGAAAGTGTTTCCCAAAAGACTTATCGCGACTGGGAGACAATCAACACT
GCGCTGGAAATTCACTATAACAATATCTTGCCGGGGAACGGAAAAAGTAAGGCAGATAAG
GTGAAGAAAGCAGTCAAAAATGATCTGCAAAAAAGCATTACTGAAATTAACGAACTTGTG
TCAAATTACAAATTGTGTTCGGATGACAATATTAAAGCGGAAACGTATATCCACGAGATC
TCGCACATTCTTAATAATTTCGAGGCGCAGGAATTAAAGTATAATCCTGAGATCCATTTG
GTGGAATCAGAACTTAAAGCTAGTGAACTGAAAAATGTCCTGGACGTTATTATGAATGCA
TTTCACTGGTGTTCTGTCTTTATGACAGAAGAACTTGTCGACAAAGACAACAACTTTTAT
GCGGAATTAGAAGAGATTTACGACGAAATTTATCCCGTTATTTCGTTATATAATTTAGTT
CGTAATTACGTGACTCAGAAACCCTACAGCACAAAAAAGATTAAATTAAACTTTGGGATT
CCGACTCTTGCTGATGGATGGAGCAAGTCCAAGGAGTACTCTAATAACGCCATTATCTTG
ATGCGTGACAACCTGTACTACCTGGGCATTTTTAACGCTAAAAACAAACCCGACAAAAAG
ATCATTGAAGGGAACACCTCGGAAAATAAGGGGGACTATAAAAAAATGATCTACAATCTG
TTGCCAGGCCCAAATAAGATGATCCCAAAGGTTTTTTTATCTTCCAAAACTGGCGTAGAA
ACTTACAAGCCGAGCGCATACATCCTTGAAGGATATAAACAAAACAAACATATCAAAAGT
TCAAAGGACTTCGATATTACGTTCTGCCATGATTTAATCGATTATTTCAAGAATTGCATC
GCGATTCACCCAGAGTGGAAAAACTTTGGGTTTGATTTTTCAGACACCAGCACTTACGAG
GATATTAGTGGATTCTATCGTGAGGTTGAACTGCAGGGCTATAAAATTGACTGGACCTAT
ATTTCTGAAAAAGATATTGATCTGCTTCAGGAGAAAGGCCAATTGTACTTATTTCAAATC
TATAACAAGGATTTCTCCAAGAAGTCCACGGGTAATGACAACTTACACACAATGTATCTG
AAGAATCTGTTTAGTGAGGAGAACTTGAAGGACATTGTGCTGAAGCTTAATGGCGAGGCC
GAAATCTTTTTTCGTAAGTCCTCCATTAAAAACCCTATTATCCATAAGAAAGGGAGTATT
CTTGTCAACCGCACGTATGAGGCCGAAGAAAAGGACCAATTCGGAAACATCCAAATTGTC
CGTAAAAATATTCCTGAGAACATTTACCAGGAGCTTTACAAGTATTTCAACGACAAGAGT
GATAAAGAACTTTCAGATGAGGCGGCGAAACTGAAGAATGTAGTGGGGCACCACGAAGCT
GCCACGAATATTGTAAAGGATTACCGTTACACCTACGACAAGTACTTTTTGCATATGCCC
ATCACAATTAATTTTAAGGCCAATAAAACTGGTTTTATCAACGATCGTATCTTACAGTAC
ATTGCTAAGGAAAAAGATCTGCACGTTATCGGTATCGATCGCGGGGAACGCAATCTGATT
TATGTTAGTGTGATTGACACGTGCGGAAATATTGTTGAGCAGAAGAGCTTTAATATCGTA
AATGGATATGACTATCAAATTAAACTGAAGCAACAGGAAGGGGCCCGCCAGATTGCCCGC
AAGGAGTGGAAAG1AAATTGGAAAGATCAAGGAGATTAAAGAAGGGTACCTTTCCCTTGT
TATCCACGAAATCTCGAAAATGGTGATCAAGTACAATGCCATTATTGCTATGGAGGATCT
GTCATATGGGTTTAAGAAAGGCCGCTTTAAGGTGGAACGTCAGGTTTACCAGAAGTTTGA
GACCATGCTTATCAATAAGCTGAATTATCTTGTCTTCAAAGACATCTCAATCACAGAGAA
CGGCGGGCTGTTAAAAGGATATCAGCTGACCTATATCCCCGACAAACTGAAAAATGTCGG
GCACCAATGCGGCTGTATTTTCTACGTGCCCGCTGCATACACATCTAAAATTGACCCAAC
GACTGGATTCGTAAATATTTTTAAGTTTAAGGATCTTACGGTAGATGCAAAGCGCGAATT
TATCAAGAAATTTGATAGTATCCGTTACGACAGCGAGAAAAACTTATTTTGTTTTACGTT
CGATTATAACAACTTCATCACGCAAAATACCGTCATGTCAAAATCTTCCTGGTCAGTCTA
TACGTATGGCGTCCGTATCAAGCGCCGCTTCGTCAACGGGCGTTTTTCAAACGAGTCAGA
TACCATCGATATCACCAAAGATATGGAAAAAACATTGGAGATGACGGACATCAATTGGCG
CGATGGTCATGACTTACGCCAGGACATTATTGACTACGAAATCGTACAACATATTTTTGA
GATTTTCCGTCTGACCGTGCAAATGCGCAACTCATTATCCGAACTTGAGGATCGTGATTA
CGACCGCTTGATCAGTCCTGTTCTGAACGAGAATAATATTTTTTACGACAGTGCCAAGGC
GGGAGACGCACTGCCCAAGGACGCTGACGCTAACGGAGCTTATTGTATTGCGTTGAAGGG
ACTTTACGAAATCAAGCAAATCACTGAAAACTGGAAGGAGGATGGTAAATTCTCACGCGA
CAAGTTGAAAATTTCGAACAAGGACTGGTTCGATTTCATCCAAAACAAGCGTTATTTAAA
ACGTCCGGCAGCGACCAAAAAAGCCGGCCAGGCGAAGAAAAAAAAAGCGTCAGGTAGCGG
CGCAGGCAGCCCGAAAAAGAAACGTAAAGTCGAGGATCCGAAAAAGAAACGTAAGGTTAT
TCCGGGCTAA
SEQ ID NO: 47
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGG
ACTAATAACTTCCAGAACTTCATCGGTATTTCATCATTACAAAAAACGCTTCGTAACGCC
TTGATCCCAACAGAAACGACCCAACAATTTATTGTAAAAAACGGCATCATCAAAGAAGAC
GAACTGCGTGGCGAAAATCGCCAAATTTTGAAGGACATTATGGATGACTATTATCGTGGG
TTTATCTCGGAGACATTATCCTCCATCGACGACATTGATTGGACGAGTCTTTTTGAGAAA
ATGGAGATCCAGCTTAAAAATGGTGATAACAAGGATACATTGATCAAGGAGCAAACCGAG
TACCGCAAGGCCATCCATAAGAAGTTCGCAAATGACGACCGCTTCAAAAATATGTTTAGT
GCCAAATTGATCTCGGATATCCTTCCTGAGTTCGTAATTCACAACAATAATTATAGCGCA
TCCGAAAAGGAGGAAAAGACTCAAGTCATTAAGCTTTTCAGTCGCTTTGCTACCTCGTTT
AAGGACTATTTCAAGAACCGCGCGAACTGCTTCTCAGCGGATGACATTTCTTCCTCGTCG
TGTCACCGCATCGTGAATGATAATGCGGAGATCTTCTTTAGTAATGCCTTGGTATACCGC
CGCATTGTTAAATCCCTGTCTAACGACGATATCAATAAGATCTCAGGAGATATGAAGGAT
AGCCTTAAAGAAATGTCTCTGGAAGAAATTTACTCCTATGAAAAGTACGGTGAGTTTATC
ACCCAAGAGGGGATTAGCTTTTATAACGATATCTGCGGGAAGGTGAATTCGTTTATGAAC
CTTTATTGTCAAAAGAATAAGGAGAATAAGAACTTATATAAGCTTCAGAAACTGCATAAA
CAAATCTTATGCATTGCCGATACTAGCTATGAAGTTCCGTATAAATTCGAGAGCGATGAA
GAAGTTTATCAGAGCGTCAATGGGTTCTTGGATAACATTTCATCAAAACACATCGTGGAA
CGTCTGCGTAAGATTGGGGATAACTACAACGGATATAATCTTGACAAAATTTATATTGTA
TCTAAATTCTATGAGTCGGTGAGTCAAAAGACCTACCGTGATTGGGAAACAATCAATACC
GCGTTAGAAATCCACTATAACAACATTCTGCCAGGGAATGGTAAAAGTAAAGCGGACAAA
GTCAAGAAGGCTGTGAAGAACGATCTGCAAAAGAGTATTACAGAGATTAACGAATTAGTC
TCCAATTATAAGTTATGCTCGGACGATAACATTAAGGCGGAGACGTATATTCATGAGATT
TCGCATATTCTTAACAACTTCGAGGCACAAGAGCTTAAGTATAACCCAGAGATTCACCTT
GTCGAATCGGAGCTGAAGGCATCGGAATTAAAAAATGTCTTAGATGTAATCATGAACGCG
TTCCATTGGTGCAGTGTTTTCATGACTGAGGAGTTAGTTGACAAGGACAATAACTTCTAC
GCAGAATTAGAAGAGATCTATGATGAGATTTATCCAGTGATTTCGCTGTATAATCTGGTA
CGTAATTACGTCACTCAAAAGCCCTACTCAACAAAAAAAATTAAGCTGAACTTCGGAATT
CCGACTCTGGCCGACGGGTGGTCCAAGTCAAAGGAGTATTCTAATAATGCTATCATCCTG
ATGCGCGATAACTTATACTATTTGGGAATTTTCAATGCCAAAAATAAACCAGATAAAAAG
ATTATCGAAGGTAATACAAGCGAGAATAAGGGTGACTATAAGAAAATGATTTACAATCTT
CTTCCAGGCCCTAACAAGATGATTCCCAAAGTTTTTTTGTCCAGTAAAACAGGGGTCGAA
ACTTACAAGCCCAGTGCCTATATCCTTGAAGGGTACAAGCAGAATAAGCACATCAAATCC
TCGAAAGACTTTGATATTACATTTTGTCATGACTTAATCGATTATTTTAAGAACTGTATC
GCAATCCATCCAGAATGGAAGAACTTCGGGTTTGATTTCTCTGATACTTCCACGTATGAG
GATATTTCCGGGTTCTACCGCGAAGTAGAGCTTCAGGGCTATAAAATTGACTGGACATAT
ATTTCAGAAAAAGACATCGATCTGTTACAAGAAAAAGGACAGTTGTATCTGTTTCAAATC
TATAATAAGGATTTCTCCAAAAAGTCAACTGGAAATGATAACTTACATACAATGTATCTG
AAAAATCTTTTTAGTGAAGAGAATTTGAAGGATATCGTGCTGAAGTTAAATGGCGAAGCA
GAGATCTTCTTCCGCAAGTCCTCGATCAAGAATCCTATCATCCACAAGAAAGGTAGTATT
CTGGTTAACCGCACGTACGAGGCCGAGGAAAAAGACCAGTTCGGTAATATCCAGATTGTA
CGTAAGAATATTCCTGAAAATATTTACCAGGAATTATACAAGTATTTTAACGACAAATCG
GATAAGGAGCTTTCAGATGAGGCCGCAAAGTTGAAGAACGTCGTAGGACACCATGAGGCC
GCTACGAATATCGTCAAGGACTACCGCTATACGTATGACAAGTACTTCCTGCACATGCCT
ATTACTATCAATTTCAAAGCTAATAAAACAGGATTCATCAATGATCGTATCCTTCAGTAC
ATTGCCAAAGAAAAAGATCTGCACGTAATCGGAATCGACCGTGGCGAACGTAATCTGATT
TACGTATCAGTTATCGACACATGTGGTAACATCGTGGAGCAGAAATCTTTTAACATTGTT
AACGGCTATGATTATCAGATTAAGCTTAAACAGCAGGAGGGGGCACGCCAAATCGCTCGT
AAAGAATGGAAGGAGATTGGAAAGATTAAAGAGATTAAAGAGGGGTACCTTTCGCTGGTT
ATTCACGAAATTTCCAAGATGGTGATTAAGTACAATGCAATCATCGCGATGGAAGATCTT
AGTTACGGATTCAAAAAGGGACGCTTCAAAGTTGAGCGTCAGGTCTACCAGAAATTTGAA
ACGATGCTGATTAACAAATTGAATTACTTGGTATTCAAAGATATCTCAATTACTGAAAAT
GGTGGCTTATTAAAGGGTTACCAGCTTACCTATATCCCGGATAAGCTGAAGAACGTGGGC
CATCAATGCGGCTGCATCTTTTACGTCCCTGCCGCATATACCTCTAAAATTGACCCCACC
ACCGGATTCGTAAATATTTTTAAATTCAAGGACCTGACGGTGGACGCCAAGCGCGAATTC
ATCAAAAAATTCGACTCAATCCGCTATGATTCCGAAAAAAATCTTTTCTGCTTTACGTTC
GATTATAATAACTTCATTACCCAAAACACGGTGATGTCAAAATCGTCCTGGAGCGTGTAT
ACTTATGGAGTGCGTATCAAGCGCCGCTTTGTTAATGGGCGCTTCAGTAACGAAAGCGAT
ACCATCGACATTACCAAAGACATGGAGAAGACGCTTGAAATGACGGATATCAATTGGCGT
GACGGACACGATCTTCGTCAGGATATCATCGACTACGAGATTGTGCAACATATCTTTGAG
ATTTTCCGTTTAACTGTTCAAATGCGTAACTCCTTGTCCGAATTGGAAGACCGTGATTAC
GACCGCTTGATTTCACCAGTGCTTAACGAGAATAACATCTTCTACGACTCCGCCAAAGCA
GGCGATGCCCTGCCAAAGGACGCTGATGCAAATGGTGCATACTGTATCGCGTTGAAGGGC
TTATACGAGATTAAGCAAATCACCGAAAATTGGAAAGAGGATGGAAAGTTCAGTCGCGAT
AAGCTGAAGATCTCTAATAAAGATTGGTTTGACTTTATCCAGAACAAACGTTATTTAAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 48
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGT
ACCAATAATTTCCAAAATTTCATCGGAATCTCATCCTTGCAAAAAACCTTGCGCAATGCT
TTGATCCCCACCGAAACCACGCAGCAGTTCATCGTGAAAAACGGCATTATCAAAGAGGAT
GAGTTGCGCGGGGAAAACCGTCAAATTCTTAAGGATATCATGGACGATTACTACCGTGGG
TTTATCAGTGAGACCCTGTCAAGCATTGACGACATTGACTGGACCAGCTTATTTGAGAAG
ATGGAGATTCAATTAAAGAACGGGGACAATAAGGACACGCTTATCAAAGAGCAGACAGAA
TACCGTAAAGCGATTCATAAGAAATTTGCAAATGACGATCGCTTCAAGAACATGTTTTCA
GCAAAATTAATCAGCGACATCCTTCCCGAATTTGTGATTCATAATAACAACTATTCGGCT
AGCGAAAAAGAGGAGAAAACTCAGGTTATTAAGCTTTTCTCGCGTTTTGCCACTTCGTTC
AAAGACTATTTTAAGAATCGCGCAAACTGCTTTTCGGCTGATGATATTTCCAGTTCTAGC
TGCCATCGTATCGTTAACGATAATGCTGAGATTTTCTTCTCTAATGCCCTGGTGTATCGT
CGTATCGTTAAATCTTTGAGCAACGACGATATTAATAAGATTTCAGGCGACATGAAGGAT
TCTTTAAAGGAGATGTCTTTAGAAGAGATTTATTCCTATGAGAAATATGGCGAGTTTATC
ACCCAAGAAGGAATTTCGTTCTACAACGACATCTGTGGCAAAGTGAACAGCTTCATGAAT
TTATACTGCCAAAAGAATAAGGAGAATAAAAATTTATATAAACTGCAGAAACTGCATAAG
CAAATTCTTTGCATTGCAGACACCTCTTATGAAGTTCCTTATAAGTTTGAATCGGACGAG
GAGGTATATCAGAGTGTGAACGGGTTCCTGGACAATATTTCATCCAAGCATATTGTTGAA
CGTTTACGCAAAATTGGAGACAATTACAATGGGTATAACCTTGACAAAATTTACATCGTG
TCGAAGTTTTACGAATCGGTAAGCCAGAAGACCTATCGTGACTGGGAAACTATCAATACC
GCCTTAGAAATTCATTACAACAATATTCTTCCTGGTAACGGCAAAAGCAAAGCCGATAAG
GTAAAGAAGGCTGTCAAGAACGACCTGCAAAAGTCTATCACAGAGATCAACGAGTTAGTC
TCTAACTACAAATTATGTTCCGACGACAATATTAAAGCCGAAACCTACATCCATGAGATC
TCACACATTCTTAACAATTTTGAGGCCCAGGAGCTGAAATATAACCCAGAAATTCACCTT
GTAGAGAGCGAATTAAAAGCCTCCGAGCTGAAGAACGTTTTGGATGTAATCATGAACGCA
TTTCATTGGTGCAGCGTATTTATGACAGAGGAGTTGGTCGACAAGGACAATAACTTTTAC
GCCGAGCTTGAAGAAATCTACGATGAAATTTACCCGGTAATTAGTTTATATAATTTAGTT
CGCAACTACGTAACTCAGAAACCCTACAGTACCAAGAAGATTAAATTGAACTTTGGGATC
CCGACACTTGCTGACGGTTGGAGTAAATCAAAAGAATACTCCAATAATGCAATTATCCTG
ATGCGCGACAATCTTTACTACTTGGGGATCTTTAACGCAAAGAACAAACCAGATAAGAAA
ATCATCGAGGGCAACACCAGCGAGAATAAAGGCGATTACAAGAAAATGATCTATAATCTT
TTGCCGGGACCGAACAAAATGATCCCAAAGGTTTTCCTGTCGTCGAAAACGGGAGTCGAG
ACATATAAACCATCTGCGTACATCTTGGAAGGTTACAAACAGAATAAGCATATTAAGTCT
AGTAAAGACTTCGACATCACCTTTTGTCATGACCTGATTGATTATTTCAAGAACTGTATT
GCTATCCATCCAGAATGGAAAAACTTCGGATTTGACTTCTCCGATACTAGCACCTACGAA
GACATTTCGGGTTTTTATCGCGAAGTAGAGCTTCAAGGGTACAAAATTGATTGGACATAT
ATTAGCGAGAAAGACATTGATTTGCTTCAAGAGAAGGGACAGTTATATTTATTCCAGATC
TACAACAAAGACTTCTCGAAGAAATCCACCGGTAATGATAATCTTCACACTATGTACCTG
AAGAATTTATTTTCAGAGGAAAATCTGAAGGACATTGTACTTAAACTTAATGGAGAAGCC
GAAATCTTCTTCCGCAAGAGTTCCATTAAAAATCCGATTATTCATAAAAAGGGAAGTATC
CTTGTGAACCGCACGTATGAGGCCGAAGAGAAGGATCAGTTTGGGAATATTCAAATTGTC
CGCAAAAACATCCCCGAGAACATCTACCAGGAACTGTATAAATACTTTAATGATAAATCT
GATAAAGAGTTATCAGACGAGGCTGCCAAACTGAAAAACGTAGTCGGTCATCATGAGGCA
GCGACCAATATTGTAAAGGACTACCGTTACACCTACGACAAGTATTTCCTTCACATGCCG
ATCACGATTAATTTTAAGGCTAACAAGACCGGCTTTATCAATGACCGCATCTTGCAGTAC
ATCGCGAAAGAGAAAGATTTACACGTCATCGGAATTGATCGTGGAGAGCGTAATCTTATC
TACGTCAGCGTCATCGACACCTGTGGAAACATTGTGGAACAAAAAAGTTTTAATATCGTA
AACGGCTACGACTATCAAATTAAACTTAAACAGCAAGAGGGAGCTCGCCAGATCGCTCGC
AAAGAGTGGAAAGAGATTGGGAAAATTAAAGAAATTAAAGAGGGTTACCTGTCGCTGGTA
ATTCACGAAATCTCGAAAATGGTCATCAAATATAATGCAATTATCGCTATGGAGGATCTG
TCCTACGGGTTCAAGAAGGGACGTTTTAAAGTAGAGCGCCAGGTGTATCAAAAATTCGAA
ACCATGTTGATCAATAAGCTTAACTATTTGGTCTTCAAAGATATTTCGATTACGGAGAAC
GGAGGTTTGTTGAAAGGATATCAGCTGACGTATATCCCAGACAAGTTGAAAAACGTGGGG
CATCAATGTGGATGTATTTTCTATGTGCCCGCGGCCTACACGAGTAAGATCGATCCTACC
ACTGGTTTCGTCAACATTTTCAAATTTAAAGATCTTACCGTGGATGCGAAGCGCGAATTT
ATTAAGAAATTTGATAGCATTCGCTATGATTCCGAAAAGAACCTGTTCTGTTTTACGTTC
GACTATAACAATTTCATTACCCAAAACACGGTGATGAGCAAATCCTCTTGGTCAGTTTAT
ACATACGGTGTACGTATCAAACGCCGTTTCGTTAACGGACGCTTTTCCAATGAGTCTGAT
ACAATCGATATCACGAAAGATATGGAAAAAACATTAGAGATGACTGATATCAACTGGCGT
GACGGGCACGACCTGCGTCAAGACATTATTGACTACGAGATTGTGCAGCATATCTTCGAA
ATCTTTCGCTTAACTGTGCAAATGCGTAACTCGTTATCCGAGTTAGAAGACCGTGACTAC
GATCGCCTGATTTCACCCGTCTTGAACGAAAATAACATCTTCTACGATTCCGCGAAGGCT
GGGGACGCATTGCCCAAGGACGCAGACGCGAATGGAGCGTACTGTATTGCGCTTAAAGGA
TTATATGAAATCAAGCAGATCACCGAAAATTGGAAGGAGGACGGGAAGTTCTCACGCGAC
AAACTGAAGATTTCAAATAAGGACTGGTTCGATTTCATTCAGAATAAGCGTTACCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 49
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAATGGT
ACGAACAACTTTCAGAACTTCATCGGCATCTCCAGCCTTCAAAAGACTTTACGCAACGCA
TTGATTCCCACGGAGACTACGCAACAGTTTATCGTAAAAAATGGTATTATCAAAGAAGAT
GAATTACGCGGGGAGAATCGCCAGATTCTTAAGGACATTATGGACGATTATTACCGTGGA
TTCATCAGTGAGACACTGAGCTCCATTGATGACATCGACTGGACGTCATTGTTTGAAAAG
ATGGAAATCCAGTTGAAAAATGGCGATAACAAAGATACATTGATTAAAGAGCAGACAGAG
TACCGCAAAGCAATTCACAAGAAATTCGCCAATGATGATCGTTTTAAGAACATGTTTAGT
GCCAAGCTTATTTCGGATATCTTACCCGAATTCGTGATTCACAACAACAATTATTCGGCA
AGTGAGAAAGAGGAAAAGACCCAGGTTATCAAATTGTTTTCGCGCTTCGCCACTTCGTTC
AAAGATTATTTCAAGAACCGTGCAAACTGTTTCTCCGCTGACGACATCAGTTCCAGCTCA
TGCCACCGTATTGTAAATGACAATGCGGAGATCTTTTTCAGTAATGCCTTAGTATATCGT
CGCATTGTAAAGAGCTTATCTAATGATGACATTAACAAGATCTCGGGTGATATGAAGGAC
TCACTTAAGGAGATGAGTCTGGAAGAGATCTACTCCTACGAAAAATACGGGGAATTCATC
ACCCAGGAGGGAATTTCATTCTACAACGATATCTGCGGCAAAGTTAACTCCTTTATGAAT
CTGTACTGTCAAAAGAACAAGGAGAATAAAAACCTGTATAAATTGCAGAAACTTCATAAA
CAAATTTTGTGTATCGCAGACACGAGTTATGAAGTACCTTATAAATTCGAATCCGACGAA
GAGGTATATCAGTCCGTAAATGGGTTCCTGGACAATATCAGTAGTAAGCACATTGTGGAA
CGCTTACGCAAAATTGGAGACAATTACAACGGGTATAACCTGGACAAAATCTACATCGTA
TCCAAATTTTATGAAAGCGTGTCTCAAAAAACTTATCGTGATTGGGAAACAATCAACACG
GCTCTTGAGATCCATTACAATAACATCTTGCCGGGTAACGGCAAATCGAAGGCAGACAAA
GTTAAAAAAGCAGTTAAGAACGACTTACAGAAAAGCATTACGGAGATTAACGAGTTAGTA
AGTAATTACAAATTATGCTCCGACGATAATATCAAAGCTGAAACCTACATCCATGAAATT
AGCCACATTTTGAACAATTTCGAAGCGCAGGAGCTGAAATATAACCCTGAAATCCATCTG
GTAGAGTCTGAGTTGAAGGCGTCAGAACTGAAAAACGTTCTTGACGTCATCATGAATGCC
TTTCACTGGTGTAGTGTTTTTATGACTGAGGAGCTTGTAGATAAGGACAACAACTTCTAT
GCTGAACTTGAAGAGATCTACGATGAAATCTACCCCGTAATCAGTCTGTATAATTTAGTT
CGTAACTACGTCACGCAGAAACCCTATTCGACTAAGAAAATTAAGCTGAACTTTGGGATC
CCTACTTTGGCAGACGGGTGGAGCAAGAGTAAAGAATACAGTAATAATGCAATTATCTTG
ATGCGCGATAACTTATATTACTTAGGTATTTTCAATGCTAAGAACAAACCTGATAAGAAG
ATTATCGAAGGAAATACGAGTGAGAATAAGGGAGACTACAAAAAGATGATTTACAACTTG
CTGCCAGGGCCTAATAAGATGATTCCAAAAGTTTTTCTGTCGAGCAAGACAGGGGTTGAA
ACTTATAAGCCATCCGCTTATATCCTTGAGGGGTACAAGCAGAATAAGCATATCAAGTCC
TCCAAAGATTTTGATATTACATTTTGCCACGACTTAATTGATTACTTCAAGAACTGCATC
GCAATCCATCCCGAATGGAAGAATTTCGGCTTCGATTTCTCAGATACGTCCACGTATGAG
GATATCTCAGGCTTTTACCGCGAAGTTGAGCTGCAAGGTTATAAAATTGATTGGACATAC
ATCTCCGAAAAAGACATTGATCTTTTACAGGAAAAGGGCCAATTATACTTATTTCAAATC
TATAACAAAGATTTTAGCAAGAAGTCCACAGGTAATGATAACCTGCATACGATGTATTTG
AAAAATCTTTTCAGTGAAGAGAATTTGAAGGATATCGTCCTGAAGCTGAACGGTGAGGCT
GAGATCTTCTTCCGCAAATCGTCTATCAAAAACCCCATCATTCACAAAAAGGGAAGTATC
TTAGTAAACCGCACTTATGAAGCGGAGGAAAAGGATCAGTTCGGGAACATCCAGATCGTG
CGCAAGAACATTCCAGAAAACATCTATCAGGAACTTTACAAATATTTCAATGACAAGTCT
GATAAAGAATTATCAGACGAGGCGGCGAAACTTAAAAATGTTGTTGGACACCACGAAGCA
GCGACGAATATTGTAAAGGATTATCGCTACACATACGATAAATACTTTTTGCACATGCCA
ATCACCATTAACTTTAAGGCGAACAAGACAGGTTTCATTAACGACCGTATTCTGCAATAT
ATCGCAAAGGAAAAAGACCTGCACGTTATTGGGATCGATCGTGGCGAACGCAATTTGATC
TACGTAAGCGTTATCGACACTTGCGGAAATATCGTTGAACAAAAAAGCTTTAATATCGTC
AATGGATACGATTACCAAATCAAGCTGAAACAACAAGAAGGGGCACGTCAGATCGCTCGT
AAAGAATGGAAAGAGATTGGTAAGATCAAAGAGATTAAAGAAGGGTATCTTTCTTTAGTA
ATTCACGAGATTTCGAAAATGGTTATTAAATACAATGCGATTATTGCTATGGAAGACTTA
AGCTACGGCTTTAAGAAAGGTCGCTTCAAAGTGGAGCGCCAAGTGTATCAGAAGTTTGAA
ACGATGTTGATTAACAAATTAAATTACCTGGTCTTTAAGGACATCAGTATCACAGAAAAT
GGGGGGTTGCTTAAAGGGTACCAGCTTACATACATCCCTGATAAACTGAAAAATGTCGGT
CATCAGTGCGGATGTATCTTCTATGTACCAGCAGCCTATACCAGTAAGATTGACCCTACT
ACTGGCTTTGTGAATATTTTTAAATTCAAGGATTTAACCGTGGACGCCAAGCGTGAATTT
ATTAAAAAATTTGATTCGATTCGCTACGACAGTGAGAAAAACCTTTTCTGCTTTACCTTT
GACTACAACAATTTTATTACCCAGAACACCGTAATGTCAAAGAGTTCGTGGTCTGTATAT
ACCTACGGTGTTCGCATCAAGCGCCGCTTCGTAAACGGGCGTTTCAGTAACGAATCTGAC
ACCATCGACATCACTAAAGATATGGAGAAGACATTGGAAATGACGGACATTAATTGGCGT
GATGGCCATGACTTACGTCAGGACATTATTGATTACGAAATTGTGCAGCATATCTTCGAG
ATTTTCCGTTTGACAGTTCAGATGCGCAACTCACTGAGTGAGTTAGAAGATCGCGATTAC
GACCGTCTGATCTCACCGGTCCTTAATGAAAACAACATTTTCTACGACTCAGCAAAGGCG
GGTGATGCCCTGCCAAAGGATGCGGACGCTAATGGCGCCTACTGCATCGCCCTGAAAGGA
TTGTATGAAATTAAGCAGATTACAGAAAATTGGAAGGAAGATGGTAAATTTAGCCGTGAT
AAATTAAAAATCTCGAACAAGGATTGGTTCGATTTTATTCAGAACAAACGTTATTTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 50
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAATGGA
ACAAATAATTTTCAAAATTTTATCGGCATCTCAAGTCTTCAAAAAACCCTTCGCAATGCC
CTGATTCCAACTGAAACAACCCAGCAATTTATCGTCAAGAACGGCATCATTAAGGAAGAC
GAGTTACGCGGGGAGAACCGTCAAATCCTGAAAGATATCATGGATGACTACTATCGTGGG
TTCATTTCGGAAACCTTGTCTTCAATCGACGACATTGACTGGACGAGTCTTTTCGAGAAA
ATGGAAATTCAGCTTAAAAATGGAGACAACAAGGATACTCTGATTAAGGAACAGACAGAA
TATCGCAAAGCTATCCACAAAAAGTTCGCTAATGATGATCGTTTCAAAAATATGTTTTCT
GCTAAATTGATTTCCGATATCTTGCCTGAATTTGTAATCCACAACAACAATTATTCTGCT
TCCGAGAAGGAAGAGAAGACCCAGGTCATTAAATTATTCAGCCGCTTTGCAACCAGCTTT
AAAGACTACTTTAAGAATCGCGCTAACTGCTTTTCGGCGGATGACATCTCATCATCATCA
TGCCACCGCATTGTGAACGACAATGCGGAGATCTTCTTTTCGAATGCGTTAGTTTATCGT
CGCATTGTCAAAAGTCTTAGCAATGATGACATCAACAAGATCTCAGGAGACATGAAAGAT
TCCTTAAAGGAGATGTCTCTTGAGGAAATCTATTCGTATGAGAAATACGGCGAGTTCATT
ACCCAGGAAGGTATTAGTTTCTACAATGATATCTGCGGCAAAGTAAATTCTTTTATGAAT
CTGTATTGCCAAAAAAACAAAGAAAACAAGAATCTTTATAAGTTACAAAAGTTACATAAG
CAAATTCTGTGCATCGCTGATACATCTTATGAGGTACCCTACAAATTTGAAAGTGATGAG
GAGGTCTATCAGAGTGTCAACGGCTTCTTAGACAACATCTCTTCCAAACATATCGTGGAA
CGCCTGCGTAAAATCGGAGATAACTACAACGGATATAACTTAGATAAAATCTACATCGTG
TCCAAGTTTTATGAAAGTGTGAGCCAAAAAACATATCGTGACTGGGAAACCATTAACACC
GCATTGGAAATTCACTATAACAACATTTTGCCAGGCAACGGGAAAAGTAAGGCGGACAAA
GTTAAGAAAGCAGTTAAAAATGACCTGCAAAAAAGCATCACTGAAATTAACGAATTGGTA
TCGAATTACAAATTATGTAGCGACGATAATATCAAAGCAGAAACTTACATTCACGAGATT
AGTCACATTTTAAATAACTTCGAGGCCCAGGAATTGAAATACAATCCCGAAATTCATTTG
GTTGAATCAGAACTGAAAGCATCAGAGTTGAAAAATGTGTTAGATGTCATTATGAATGCG
TTTCATTGGTGCTCTGTGTTCATGACCGAGGAACTGGTTGATAAAGATAACAACTTTTAC
GCTGAATTGGAGGAGATTTACGATGAGATTTACCCGGTCATTTCGCTTTATAACTTAGTG
CGCAATTATGTGACGCAGAAACCATATTCCACGAAGAAAATCAAACTTAATTTTGGCATC
CCTACTCTGGCTGATGGTTGGTCGAAATCGAAAGAGTACAGCAACAACGCGATCATTCTT
ATGCGTGACAATCTTTACTATTTGGGCATTTTTAATGCCAAGAATAAGCCAGATAAGAAA
ATCATTGAGGGGAATACTTCCGAGAATAAGGGGGATTACAAAAAGATGATCTATAACTTG
CTGCCCGGCCCCAACAAAATGATTCCTAAGGTTTTCTTGTCAAGCAAGACGGGCGTCGAA
ACATATAAGCCGTCAGCTTATATTCTGGAAGGCTATAAACAGAATAAGCACATCAAGTCT
TCCAAGGACTTTGACATCACTTTTTGCCACGATTTGATCGACTACTTTAAGAACTGTATT
GCGATTCATCCGGAATGGAAGAACTTCGGTTTCGACTTTTCCGATACCTCAACATACGAG
GATATCAGCGGCTTCTACCGTGAAGTCGAGCTTCAAGGCTACAAGATCGATTGGACATAT
ATTTCAGAGAAGGACATTGATTTGTTACAAGAGAAAGGTCAACTTTACTTATTTCAGATC
TATAACAAAGACTTTTCGAAGAAATCGACAGGAAACGATAACTTACACACTATGTATTTA
AAAAATCTGTTTTCGGAGGAAAACCTGAAAGATATTGTGCTGAAACTTAACGGCGAGGCA
GAGATCTTTTTCCGTAAAAGCTCAATCAAGAATCCTATCATCCATAAAAAAGGTAGTATT
CTTGTCAACCGCACATATGAAGCGGAGGAGAAGGACCAATTCGGAAACATCCAAATTGTC
CGTAAGAATATTCCGGAGAACATTTACCAAGAGTTGTATAAATACTTTAACGATAAGTCA
GATAAGGAACTTAGCGATGAGGCGGCGAAGCTTAAAAACGTAGTTGGGCATCATGAAGCT
GCTACCAACATTGTAAAAGATTACCGTTACACCTATGACAAGTATTTCTTGCACATGCCC
ATTACGATCAATTTCAAAGCAAATAAGACAGGCTTTATCAATGATCGCATCCTGCAGTAC
ATTGCTAAAGAGAAGGATTTGCATGTTATCGGTATTGATCGCGGAGAGCGCAATTTGATC
TACGTCTCCGTAATCGACACTTGCGGTAACATTGTTGAGCAGAAGTCGTTCAACATCGTT
AATGGTTATGATTACCAAATCAAGCTGAAGCAGCAAGAGGGTGCCCGCCAGATCGCGCGT
AAGGAATGGAAAGAAATCGGGAAAATTAAAGAGATCAAAGAAGGCTATTTGTCTCTGGTA
ATTCACGAAATCAGCAAGATGGTGATCAAGTATAACGCGATCATTGCGATGGAGGATCTT
TCTTATGGCTTCAAGAAAGGGCGCTTTAAAGTCGAACGCCAGGTCTACCAGAAATTTGAG
ACAATGCTTATCAACAAGCTTAACTATCTTGTATTTAAGGATATTTCCATCACTGAGAAC
GGAGGACTTTTAAAGGGGTACCAACTGACGTACATTCCTGATAAGCTGAAGAACGTTGGT
CATCAATGCGGATGCATCTTCTATGTGCCAGCGGCTTACACCTCCAAAATCGATCCCACT
ACAGGCTTTGTCAATATCTTCAAATTCAAGGATTTGACCGTTGACGCGAAGCGCGAGTTT
ATCAAGAAGTTTGATAGCATTCGCTACGACAGCGAAAAAAATTTATTTTGTTTTACTTTC
GACTACAATAACTTTATTACTCAGAACACTGTCATGTCAAAGAGTTCGTGGAGTGTCTAC
ACGTACGGAGTACGTATTAAGCGCCGTTTCGTCAACGGACGCTTCTCAAACGAAAGCGAC
ACGATCGACATCACCAAAGACATGGAAAAAACTCTTGAGATGACGGATATCAATTGGCGC
GACGGCCATGACCTGCGTCAGGATATCATTGATTACGAGATCGTTCAGCACATCTTCGAA
ATCTTCCGCCTTACCGTCCAGATGCGCAACAGTTTAAGCGAGCTTGAAGACCGCGACTAC
GATCGTTTGATTAGCCCCGTTCTGAACGAGAATAATATTTTCTACGACAGCGCAAAGGCC
GGTGATGCTTTGCCAAAGGACGCAGACGCGAATGGAGCCTACTGCATCGCCCTGAAGGGC
TTATATGAGATTAAGCAAATTACCGAAAATTGGAAGGAAGATGGTAAGTTCTCCCGTGAT
AAGCTTAAAATTAGCAATAAGGATTGGTTCGACTTCATCCAGAACAAACGTTACCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 51
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGA
ACAAACAATTTCCAAAACTTCATCGGTATCTCTTCGTTGCAGAAGACTCTGCGTAATGCT
TTGATCCCGACGGAGACAACCCAACAATTTATCGTCAAAAACGGTATTATTAAGGAGGAC
GAGTTACGTGGAGAAAATCGTCAAATCCTTAAGGACATCATGGACGATTATTATCGCGGG
TTTATTTCTGAAACCCTGAGCAGTATCGATGATATCGACTGGACCTCACTTTTTGAGAAA
ATGGAGATCCAGTTGAAGAACGGTGATAACAAAGACACTCTGATCAAAGAGCAAACTGAA
TACCGCAAGGCAATTCACAAAAAGTTCGCCAACGACGACCGTTTCAAGAATATGTTCTCA
GCTAAGTTAATCAGCGACATTTTGCCAGAGTTCGTTATCCACAACAATAATTATAGTGCT
TCAGAGAAGGAGGAAAAAACCCAAGTGATTAAACTTTTTTCGCGCTTTGCAACCTCATTC
AAGGACTACTTCAAGAATCGCGCGAATTGCTTCAGTGCGGACGACATTTCTTCTTCAAGT
TGCCATCGTATCGTTAACGATAACGCGGAAATTTTCTTCTCTAATGCTTTGGTGTATCGC
CGCATTGTAAAATCGCTTAGTAACGATGACATTAATAAGATCTCAGGTGATATGAAAGAT
TCATTGAAGGAAATGAGCTTGGAAGAGATTTACAGTTACGAAAAATATGGAGAATTTATT
ACTCAGGAAGGCATCTCATTCTATAACGATATCTGCGGGAAGGTAAATTCGTTTATGAAC
TTATATTGCCAGAAAAATAAAGAGAATAAAAATTTGTATAAGCTTCAGAAGTTGCACAAA
CAGATCCTGTGCATTGCAGACACCTCGTATGAGGTTCCGTATAAATTTGAGTCCGATGAA
GAAGTGTATCAGTCTGTGAATGGTTTCTTAGATAATATCTCTTCCAAGCATATTGTCGAA
CGCCTGCGCAAAATTGGTGATAACTATAACGGATACAATCTGGATAAAATTTACATCGTT
TCTAAATTTTACGAGTCAGTCTCGCAGAAGACCTACCGCGACTGGGAAACAATTAACACG
GCATTGGAGATTCACTACAATAATATCTTGCCTGGTAACGGTAAGTCTAAGGCAGATAAG
GTAAAAAAAGCTGTGAAAAACGACCTTCAGAAAAGCATCACGGAGATTAATGAGCTGGTG
AGTAATTACAAATTATGTTCAGACGATAATATTAAAGCTGAAACGTATATCCATGAAATC
TCGCATATCTTGAACAACTTCGAGGCCCAAGAACTTAAATATAACCCCGAAATCCATTTA
GTCGAGTCTGAATTGAAAGCGTCGGAATTAAAAAACGTCTTAGACGTCATTATGAACGCG
TTTCACTGGTGTTCAGTTTTCATGACCGAAGAGCTGGTCGACAAAGACAACAACTTCTAT
GCGGAATTGGAGGAAATCTATGATGAAATCTACCCTGTTATTTCACTGTATAACCTTGTG
CGCAACTATGTCACTCAGAAGCCGTATTCGACCAAAAAAATTAAATTGAATTTCGGTATC
CCTACTCTTGCAGACGGATGGAGTAAAAGCAAGGAATACAGTAATAACGCCATTATTCTT
ATGCGCGACAATTTATACTACCTGGGCATCTTTAACGCAAAGAATAAGCCGGATAAGAAG
ATTATTGAGGGTAACACCAGTGAGAACAAGGGCGACTATAAGAAGATGATCTATAACTTA
TTGCCAGGTCCAAATAAAATGATCCCAAAAGTATTCTTATCATCAAAGACGGGAGTTGAA
ACCTATAAGCCTAGTGCCTATATTCTTGAGGGATATAAACAGAACAAGCACATTAAGTCG
TCTAAGGATTTTGACATTACGTTCTGCCATGACTTAATCGACTATTTTAAAAACTGTATT
GCGATTCACCCCGAATGGAAGAATTTTGGATTCGATTTTTCGGATACCTCGACCTATGAA
GATATTTCGGGATTTTATCGTGAAGTGGAGTTGCAAGGCTATAAAATCGATTGGACCTAT
ATCTCAGAAAAAGACATTGATTTATTACAGGAAAAGGGACAACTGTACCTTTTCCAAATT
TATAACAAGGACTTTTCTAAAAAGTCCACAGGAAATGATAACCTTCACACCATGTACCTG
AAGAACCTTTTCTCAGAGGAAAACCTGAAGGACATTGTCCTTAAGTTAAATGGAGAAGCG
GAGATCTTTTTCCGTAAATCTAGTATCAAGAATCCGATTATCCATAAAAAAGGTTCGATT
TTGGTAAATCGCACCTATGAAGCGGAAGAGAAAGATCAATTTGGTAACATCCAGATCGTG
CGCAAGAATATCCCGGAGAACATTTACCAAGAGCTGTATAAGTACTTCAATGATAAGTCT
GATAAGGAACTGTCAGATGAAGCTGCGAAATTGAAGAACGTGGTTGGGCATCATGAAGCC
GCTACCAATATCGTCAAGGATTACCGTTATACCTATGACAAATATTTCTTACACATGCCG
ATTACGATCAATTTTAAGGCAAACAAGACAGGATTCATCAACGACCGTATCTTGCAGTAT
ATTGCCAAAGAGAAGGATCTGCATGTGATCGGTATTGACCGCGGGGAGCGCAATTTAATC
TATGTATCGGTGATCGATACTTGTGGTAACATCGTAGAACAAAAGAGCTTTAACATCGTG
AATGGTTACGACTATCAGATCAAGCTGAAACAACAGGAAGGAGCCCGCCAGATCGCTCGC
AAGGAATGGAAAGAAATCGGGAAAATTAAGGAAATCAAGGAAGGCTACCTTTCATTGGTC
ATTCACGAAATTTCGAAAATGGTAATTAAGTACAACGCGATCATCGCCATGGAGGACCTT
TCGTACGGATTTAAGAAGGGTCGTTTCAAAGTTGAGCGCCAGGTATACCAAAAATTCGAG
ACTATGCTTATCAACAAACTTAACTACTTGGTCTTTAAGGACATTTCTATTACCGAAAAC
GGCGGCTTACTTAAAGGCTATCAATTGACATATATTCCCGACAAACTGAAGAATGTTGGA
CATCAATGCGGGTGTATTTTCTATGTGCCGGCAGCTTACACTAGTAAGATCGACCCTACA
ACCGGGTTCGTAAACATTTTTAAATTCAAAGACTTAACAGTCGATGCGAAGCGTGAATTT
ATTAAGAAGTTTGATAGTATCCGCTATGACAGTGAAAAGAACTTGTTTTGCTTTACGTTC
GACTACAATAACTTTATTACACAGAACACGGTCATGTCTAAATCATCATGGTCGGTTTAC
ACATATGGGGTGCGCATCAAGCGTCGCTTTGTAAATGGCCGTTTTAGTAATGAGAGCGAC
ACAATCGACATCACAAAGGATATGGAGAAAACTCTTGAGATGACAGACATCAATTGGCGT
GACGGTCATGACTTACGCCAAGATATCATCGACTACGAAATCGTACAGCATATTTTTGAG
ATTTTTCGTCTTACTGTGCAAATGCGTAATTCTTTATCCGAACTGGAAGATCGTGATTAC
GACCGCTTGATTAGTCCCGTCTTAAATGAGAACAATATTTTCTATGATTCTGCGAAAGCC
GGAGATGCACTGCCCAAAGACGCTGATGCCAATGGCGCGTATTGCATTGCATTAAAAGGA
TTATATGAGATTAAACAGATTACCGAAAATTGGAAAGAGGACGGTAAATTCTCACGCGAT
AAATTGAAGATTTCTAACAAGGACTGGTTCGACTTTATCCAAAATAAACGTTATCTTAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 52
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAACGGT
ACCAACAACTTTCAGAATTTCATTGGCATTAGCTCGCTTCAAAAAACTTTACGCAATGCT
CTTATTCCGACTGAGACGACACAACAGTTTATCGTTAAGAATGGCATCATCAAAGAAGAT
GAATTACGCGGAGAAAACCGCCAGATCCTGAAAGACATTATGGACGATTATTACCGTGGG
TTCATCTCCGAGACGTTGTCATCGATCGATGACATCGACTGGACGTCACTTTTTGAAAAA
ATGGAGATCCAGTTAAAGAACGGTGACAATAAGGATACATTGATCAAAGAACAGACCGAG
TACCGTAAAGCGATTCATAAAAAGTTTGCGAACGATGATCGCTTCAAGAATATGTTTTCT
GCGAAATTAATTTCCGACATTTTACCTGAATTTGTTATTCATAATAACAACTACTCGGCG
TCTGAGAAAGAGGAGAAAACCCAAGTGATTAAACTTTTTTCACGTTTCGCAACGTCGTTC
AAAGACTATTTTAAAAATCGTGCTAATTGCTTTAGCGCGGATGACATCAGCTCTAGTTCA
TGTCATCGCATTGTCAACGATAATGCTGAGATCTTTTTCAGTAATGCGTTAGTGTACCGT
CGTATTGTGAAGTCCTTATCTAATGATGATATCAATAAGATCAGCGGGGATATGAAGGAC
TCACTTAAGGAGATGAGCTTGGAGGAAATCTATTCCTATGAGAAGTATGGTGAGTTTATT
ACGCAAGAAGGAATTAGCTTTTACAACGATATCTGTGGAAAGGTGAATTCGTTTATGAAT
TTGTATTGCCAGAAAAATAAGGAGAACAAGAACCTTTATAAATTGCAAAAGTTACACAAG
CAAATCCTGTGCATTGCAGATACTTCCTACGAGGTGCCTTACAAGTTTGAATCCGACGAA
GAGGTCTACCAATCTGTAAACGGTTTCTTAGATAATATTAGTTCCAAGCATATTGTGGAG
CGCCTTCGTAAAATTGGCGATAATTACAACGGTTACAATTTAGACAAAATTTACATTGTC
AGTAAATTCTACGAGTCCGTATCTCAAAAGACGTATCGTGATTGGGAGACTATCAATACG
GCCCTGGAGATCCACTACAACAATATCTTGCCCGGTAATGGTAAGTCGAAGGCCGATAAA
GTTAAGAAAGCGGTGAAAAATGACTTACAGAAGTCAATCACCGAAATTAACGAATTGGTG
TCCAATTATAAATTGTGTTCAGATGATAATATCAAAGCCGAGACCTACATTCATGAGATT
TCCCATATCTTAAATAATTTCGAGGCGCAAGAGCTTAAGTATAACCCAGAAATCCACCTG
GTAGAATCTGAGTTGAAGGCGTCAGAGTTAAAAAATGTTTTAGATGTCATTATGAACGCG
TTTCACTGGTGCTCCGTATTTATGACGGAGGAATTAGTAGATAAAGACAACAATTTCTAT
GCCGAACTTGAGGAAATCTATGATGAGATCTATCCCGTCATTAGCCTGTATAACTTGGTC
CGCAACTATGTTACCCAAAAACCGTACAGTACCAAGAAGATTAAGCTGAATTTCGGCATT
CCTACACTGGCTGATGGTTGGAGTAAATCGAAGGAATATTCGAATAACGCGATTATCTTG
ATGCGCGACAACTTATACTATTTGGGGATCTTTAACGCCAAAAACAAACCGGATAAGAAG
ATTATTGAGGGAAACACATCAGAGAACAAAGGCGACTACAAAAAAATGATTTACAACTTG
TTACCGGGGCCTAACAAAATGATCCCGAAGGTGTTCTTATCCAGTAAAACAGGCGTTGAG
ACCTACAAACCTTCCGCATACATCCTGGAAGGGTATAAGCAGAACAAGCACATTAAGTCC
AGCAAGGATTTCGATATTACCTTCTGTCATGATTTAATTGACTATTTCAAGAACTGTATT
GCAATCCACCCCGAGTGGAAGAACTTCGGATTCGACTTCTCAGATACGAGCACATATGAG
GACATCTCGGGGTTCTATCGTGAAGTAGAACTGCAGGGATATAAAATTGATTGGACATAT
ATTTCCGAAAAAGACATCGACCTTTTACAAGAGAAGGGTCAACTTTACTTGTTCCAAATT
TACAATAAAGACTTCTCAAAAAAAAGCACGGGTAACGATAATTTACACACTATGTATTTA
AAGAACCTTTTCTCGGAAGAGAATTTAAAGGATATCGTATTGAAGTTGAATGGAGAAGCG
GAGATCTTCTTCCGTAAGTCCAGTATTAAAAACCCTATTATTCACAAGAAGGGATCGATT
TTAGTTAACCGCACATACGAGGCCGAAGAGAAGGACCAATTTGGGAACATTCAAATTGTC
CGCAAAAACATCCCTGAGAACATTTATCAAGAGCTTTATAAGTACTTTAACGATAAGTCC
GATAAGGAATTGTCAGATGAGGCGGCAAAGTTGAAGAATGTCGTGGGGCATCATGAAGCT
GCCACCAACATTGTGAAGGACTACCGCTACACTTACGACAAATACTTCCTGCACATGCCC
ATTACGATCAATTTTAAGGCCAATAAGACAGGCTTTATTAACGACCGTATTCTTCAATAT
ATCGCTAAGGAGAAGGACCTTCATGTGATTGGGATCGACCGCGGAGAACGTAATTTAATT
TATGTGTCCGTCATCGATACGTGTGGAAATATCGTGGAACAGAAATCATTCAATATCGTG
AATGGCTATGATTACCAGATCAAATTAAAACAGCAGGAGGGCGCTCGCCAAATTGCGCGT
AAGGAATGGAAAGAGATCGGAAAAATCAAAGAAATCAAAGAAGGATATTTGTCATTGGTG
ATCCATGAGATTTCAAAAATGGTAATTAAATATAATGCAATTATCGCAATGGAAGACCTG
TCCTATGGTTTTAAGAAGGGTCGTTTCAAGGTAGAACGCCAAGTGTATCAAAAGTTCGAG
ACGATGCTGATCAATAAGCTGAATTATCTTGTGTTTAAGGACATTAGCATCACGGAAAAT
GGAGGGCTGTTGAAAGGCTATCAACTGACGTATATCCCTGACAAGCTGAAAAATGTTGGC
CATCAGTGCGGGTGCATTTTCTACGTCCCCGCGGCGTATACAAGCAAGATCGATCCTACT
ACGGGATTCGTAAATATTTTTAAATTCAAAGACTTAACCGTGGACGCCAAGCGCGAATTC
ATTAAGAAGTTTGATAGCATTCGCTACGATTCAGAAAAAAATCTTTTCTGTTTTACGTTC
GATTACAACAATTTTATCACCCAGAACACAGTGATGAGCAAGTCATCCTGGTCTGTCTAT
ACCTACGGTGTCCGTATCAAACGCCGCTTCGTCAACGGACGCTTCTCTAATGAATCTGAT
ACCATTGACATCACCAAGGACATGGAAAAGACACTTGAGATGACAGATATTAACTGGCGT
GACGGACATGACCTGCGTCAGGACATCATCGATTATGAGATTGTTCAGCATATCTTCGAG
ATCTTCCGCCTGACAGTACAAATGCGCAATTCACTGTCAGAACTTGAAGACCGCGACTAT
GACCGCCTGATCTCTCCAGTATTAAATGAGAACAATATCTTTTATGACAGTGCTAAGGCC
GGCGATGCCCTTCCGAAAGATGCTGATGCTAACGGAGCTTATTGTATTGCATTAAAGGGT
CTTTATGAGATCAAGCAAATTACCGAGAATTGGAAGGAGGATGGCAAATTCTCGCGCGAC
AAACTGAAAATCAGTAACAAGGACTGGTTCGATTTTATTCAGAATAAACGTTACCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 53
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAACGGA
ACGAACAACTTCCAGAACTTCATCGGCATCAGTTCTTTACAAAAAACCCTGCGTAACGCC
CTTATTCCGACTGAGACAACACAACAGTTCATCGTTAAAAACGGAATTATCAAAGAGGAC
GAGTTGCGCGGCGAGAATCGCCAAATTTTGAAAGATATTATGGACGACTATTATCGTGGT
TTTATTTCAGAAACACTGAGTTCGATTGACGATATCGATTGGACGAGCCTGTTTGAGAAA
ATGGAAATCCAGTTGAAAAATGGCGATAATAAAGACACTTTAATCAAAGAACAAACCGAG
TATCGTAAAGCGATCCATAAAAAGTTCGCTAATGACGATCGTTTTAAGAATATGTTCAGT
GCGAAACTGATTTCAGACATTTTGCCCGAGTTCGTGATCCATAATAACAACTATTCCGCC
TCGGAAAAGGAAGAAAAAACCCAGGTGATTAAGCTGTTCAGTCGCTTCGCAACATCTTTC
AAGGATTATTTCAAGAATCGCGCGAATTGCTTCAGTGCGGACGATATTTCTAGTTCAAGC
TGCCATCGTATCGTTAATGATAACGCGGAGATTTTTTTTAGCAATGCTCTGGTGTACCGC
CGCATTGTTAAGTCACTGTCCAACGATGATATTAACAAGATCTCAGGAGACATGAAAGAC
TCGCTTAAAGAGATGAGTCTGGAAGAGATCTATTCTTATGAGAAGTATGGCGAGTTTATT
ACCCAAGAAGGAATCTCATTCTACAATGATATTTGTGGAAAGGTGAACAGCTTTATGAAT
CTTTACTGCCAAAAAAACAAGGAGAATAAGAATCTTTACAAACTTCAGAAGTTACATAAA
CAGATTTTGTGTATTGCGGATACGTCTTATGAAGTCCCCTACAAATTTGAATCGGATGAA
GAGGTATACCAAAGTGTGAACGGATTCTTGGACAATATTTCTTCTAAACATATTGTTGAA
CGCTTACGTAAGATCGGGGATAACTACAATGGCTACAATCTTGACAAAATCTACATTGTT
AGCAAATTCTACGAGAGTGTCAGCCAAAAGACGTACCGCGATTGGGAAACAATTAATACT
GCGCTTGAGATTCACTATAATAACATTTTACCAGGCAACGGCAAGTCCAAGGCGGATAAA
GTTAAAAAAGCTGTTAAAAACGATTTGCAAAAATCTATCACAGAAATTAACGAGTTAGTT
AGTAACTACAAACTGTGCTCCGATGACAACATTAAGGCTGAGACGTATATCCATGAGATC
TCTCACATCTTAAACAATTTTGAAGCTCAAGAACTTAAGTACAATCCGGAAATCCACCTG
GTGGAATCCGAGCTGAAGGCTAGCGAACTGAAGAACGTATTGGACGTGATCATGAACGCG
TTCCACTGGTGTTCTGTCTTTATGACGGAAGAGCTTGTCGACAAAGATAATAACTTTTAC
GCGGAACTTGAGGAAATTTACGATGAGATTTACCCAGTTATTTCATTGTATAACCTTGTC
CGTAATTACGTGACCCAAAAGCCTTATAGTACGAAAAAAATCAAATTAAATTTTGGAATC
CCAACACTGGCTGACGGTTGGAGCAAATCTAAGGAGTATTCTAATAACGCAATCATCTTA
ATGCGTGACAACCTGTATTATTTGGGTATCTTCAATGCCAAAAATAAGCCTGACAAAAAG
ATTATCGAAGGAAATACTTCGGAGAATAAGGGGGATTACAAAAAAATGATTTACAATTTG
CTGCCCGGGCCGAACAAGATGATCCCCAAAGTGTTCTTATCCTCGAAGACTGGTGTAGAA
ACATACAAGCCAAGCGCATACATTCTGGAGGGTTACAAGCAAAACAAACACATCAAATCT
TCAAAAGACTTTGACATTACATTTTGCCATGATCTTATTGACTACTTCAAAAACTGCATT
GCTATTCACCCCGAGTGGAAGAACTTTGGGTTTGACTTCAGCGACACGTCTACGTATGAG
GACATCTCCGGGTTCTACCGTGAAGTTGAGTTACAAGGGTATAAGATTGACTGGACGTAT
ATTTCAGAGAAAGATATCGATCTTTTGCAGGAAAAGGGCCAGTTATATTTATTCCAGATT
TACAACAAGGACTTTAGTAAGAAGTCAACAGGAAATGACAACTTGCATACGATGTATTTG
AAAAATCTTTTTTCTGAGGAAAATCTTAAGGACATCGTACTGAAATTGAATGGCGAGGCT
GAAATCTTCTTCCGTAAATCCTCCATTAAGAATCCCATTATCCACAAAAAGGGGTCTATC
CTGGTGAATCGTACCTACGAGGCAGAGGAGAAGGATCAATTCGGAAATATTCAGATTGTT
CGTAAGAACATCCCCGAGAACATTTATCAAGAATTGTATAAGTACTTTAATGACAAATCT
GACAAAGAGTTATCCGACGAAGCTGCGAAACTGAAAAACGTTGTTGGTCACCACGAGGCC
GCCACTAATATCGTAAAAGACTACCGTTATACCTATGACAAGTACTTTTTGCACATGCCG
ATCACTATCAACTTCAAGGCGAATAAGACGGGCTTCATTAACGATCGTATCCTGCAATAC
ATCGCCAAGGAGAAGGACCTTCACGTCATTGGGATTGACCGTGGTGAGCGTAACCTGATT
TATGTAAGCGTCATTGATACCTGCGGTAATATCGTCGAACAGAAAAGTTTCAACATTGTA
AATGGATATGACTATCAGATCAAACTTAAGCAGCAGGAGGGTGCACGCCAGATTGCCCGC
AAGGAATGGAAGGAGATTGGGAAGATTAAGGAAATTAAAGAAGGTTACTTATCACTGGTT
ATTCACGAGATCAGTAAAATGGTAATCAAATATAACGCGATCATTGCCATGGAGGATCTG
AGCTATGGCTTTAAAAAGGGCCGTTTCAAAGTCGAGCGCCAGGTATATCAAAAGTTTGAA
ACAATGCTGATTAACAAATTAAACTATCTGGTTTTCAAAGATATTTCGATCACTGAAAAT
GGCGGGCTGTTGAAGGGATACCAACTTACATACATCCCTGACAAACTGAAAAATGTCGGT
CACCAATGTGGATGTATCTTTTATGTACCAGCAGCGTATACGAGCAAAATCGATCCAACT
ACGGGTTTTGTGAACATCTTTAAGTTCAAGGATTTGACAGTAGATGCCAAACGCGAGTTC
ATTAAAAAATTTGATTCAATTCGCTACGATTCAGAGAAAAATCTTTTTTGTTTCACGTTC
GATTACAATAATTTCATTACGCAGAACACAGTAATGTCAAAGTCAAGCTGGTCGGTCTAC
ACGTATGGAGTCCGTATTAAACGTCGTTTTGTAAACGGCCGTTTCTCAAATGAATCAGAT
ACAATTGATATTACGAAGGATATGGAGAAGACATTAGAGATGACTGACATTAACTGGCGC
GACGGACATGATCTTCGTCAGGACATTATTGATTATGAGATTGTACAGCATATCTTTGAG
ATCTTCCGCCTGACCGTTCAGATGCGCAATTCGTTGTCCGAGTTAGAAGACCGCGATTAC
GACCGTTTAATCAGTCCCGTCTTAAACGAAAATAACATCTTCTACGATTCAGCCAAGGCA
GGCGATGCCTTGCCAAAGGATGCTGACGCAAATGGCGCATACTGTATTGCGTTGAAAGGC
CTTTATGAAATCAAGCAAATTACCGAAAACTGGAAAGAAGACGGAAAATTCTCCCGTGAT
AAGTTGAAAATCTCTAATAAGGATTGGTTCGATTTCATCCAAAATAAACGCTATTTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 54
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGA
ACTAATAATTTCCAAAATTTTATAGGCATCTCTTCTTTACAGAAGACTCTTCGTAACGCC
CTAATCCCGACTGAGACCACACAACAATTCATAGTGAAAAATGGGATCATTAAAGAAGAC
GAGCTGCGTGGGGAGAACAGGCAGATCCTAAAAGACATAATGGACGATTATTATAGAGGG
TTCATCTCAGAGACATTATCTAGCATCGACGACATTGACTGGACCTCCCTGTTTGAAAAA
ATGGAAATCCAGCTGAAGAATGGTGACAATAAAGACACATTAATAAAAGAACAAACAGAG
TACAGGAAAGCCATCCACAAGAAGTTCGCAAACGATGACAGATTCAAAAATATGTTCAGT
GCGAAGCTAATATCCGACATCTTACCAGAGTTTGTAATACACAATAACAATTACAGCGCG
AGCGAAAAGGAAGAGAAAACGCAAGTAATTAAGCTTTTTAGTAGGTTCGCTACCTCTTTC
AAAGATTACTTCAAAAATCGTGCTAACTGCTTCTCAGCCGACGACATATCTTCAAGTTCC
TGTCACCGTATCGTGAATGATAACGCTGAGATATTCTTCTCAAACGCCCTTGTATACCGT
AGGATCGTAAAGTCCTTATCTAACGATGATATAAACAAGATCAGTGGAGACATGAAAGAC
AGCCTTAAAGAGATGTCTCTAGAAGAAATTTACTCCTATGAAAAGTATGGGGAGTTTATA
ACACAGGAGGGGATCAGCTTCTACAACGACATCTGCGGAAAGGTGAACAGTTTCATGAAT
CTTTACTGCCAGAAGAATAAAGAGAACAAAAATCTTTATAAGCTTCAAAAGTTGCACAAA
CAAATACTGTGCATTGCCGATACATCATATGAGGTCCCCTATAAGTTCGAATCTGATGAG
GAAGTTTATCAATCTGTTAACGGCTTTCTAGACAATATCAGCTCAAAACACATCGTAGAA
AGACTGAGGAAAATAGGTGATAATTATAATGGATACAACTTGGATAAAATATATATAGTC
TCTAAATTTTACGAGTCAGTATCCCAGAAAACGTATAGGGATTGGGAGACCATCAACACG
GCGTTAGAGATTCATTACAATAACATCTTACCGGGAAACGGAAAAAGTAAGGCGGACAAA
GTAAAGAAAGCCGTTAAAAATGACTTACAAAAGAGTATAACAGAAATAAACGAACTAGTA
AGCAACTACAAGCTTTGTTCCGATGATAATATCAAGGCCGAGACATATATCCATGAGATC
TCCCACATTCTAAACAATTTCGAAGCGCAAGAACTTAAATATAATCCCGAAATCCACCTG
GTGGAAAGTGAACTAAAGGCTAGTGAGTTAAAGAACGTTCTTGATGTTATCATGAACGCC
TTCCATTGGTGCTCTGTTTTTATGACCGAGGAGTTGGTTGATAAAGATAATAATTTCTAC
GCTGAATTAGAGGAGATATACGACGAAATCTACCCAGTGATTTCACTATACAACTTGGTC
AGGAACTATGTTACACAAAAGCCGTACAGCACTAAGAAAATTAAGCTAAATTTCGGTATC
CCCACGTTAGCCGACGGGTGGAGCAAGTCCAAAGAATATTCCAACAATGCGATTATTTTA
ATGCGTGACAATCTTTATTACCTTGGCATCTTCAATGCCAAAAACAAACCTGACAAAAAG
ATTATAGAAGGTAATACGTCCGAGAACAAAGGCGATTACAAGAAGATGATTTATAACCTA
CTGCCCGGACCAAACAAAATGATCCCCAAAGTTTTTCTTAGTTCTAAAACCGGCGTAGAG
ACGTATAAACCTTCTGCCTATATCTTAGAGGGATATAAGCAGAACAAACATATCAAATCT
TCCAAGGACTTTGATATTACATTCTGCCACGATTTAATTGACTACTTCAAAAATTGCATA
GCGATACATCCGGAGTGGAAGAACTTTGGCTTCGACTTCAGTGATACATCCACCTATGAG
GATATATCAGGCTTCTATCGTGAGGTCGAATTGCAAGGGTACAAAATCGATTGGACGTAT
ATATCCGAGAAAGACATAGACCTTCTTCAAGAAAAGGGGCAGTTATATTTATTCCAAATA
TACAACAAGGACTTCAGTAAGAAGTCAACAGGTAATGACAACTTACACACCATGTACTTG
AAAAATTTATTTTCTGAAGAAAACCTAAAGGACATTGTACTAAAACTGAACGGGGAGGCA
GAAATTTTTTTTAGAAAGAGCAGCATAAAAAACCCAATAATTCATAAGAAAGGAAGCATT
TTAGTTAATAGGACGTACGAGGCAGAGGAAAAGGACCAGTTTGGCAATATCCAGATCGTA
AGGAAAAATATTCCTGAAAACATATATCAGGAACTATATAAATACTTTAACGACAAATCC
GACAAAGAATTATCCGACGAGGCTGCAAAGCTGAAGAACGTCGTAGGGCACCATGAGGCA
GCGACTAATATTGTGAAAGACTATAGGTATACATACGACAAATACTTTCTGCACATGCCC
ATCACGATTAACTTCAAGGCGAACAAGACGGGATTCATTAACGACCGTATATTACAATAT
ATTGCTAAGGAGAAAGATCTGCATGTAATAGGTATCGACAGAGGCGAACGTAATTTAATC
TACGTGTCCGTCATCGACACGTGCGGGAACATCGTAGAGCAAAAGAGTTTTAATATAGTA
AATGGCTATGATTACCAAATTAAGCTAAAGCAGCAAGAAGGAGCAAGACAGATAGCTAGG
AAAGAATGGAAGGAGATAGGAAAAATAAAGGAGATCAAGGAGGGGTATCTTAGCCTAGTA
ATTCATGAAATATCTAAGATGGTTATCAAATACAACGCTATCATAGCGATGGAAGACTTA
TCTTATGGTTTCAAGAAAGGAAGGTTCAAAGTAGAGCGTCAAGTTTATCAAAAGTTCGAA
ACGATGTTGATTAATAAACTAAACTATTTGGTATTTAAAGATATATCTATCACCGAGAAT
GGTGGTCTACTAAAGGGTTACCAGCTTACATACATACCGGACAAACTTAAAAACGTCGGA
CATCAGTGTGGATGCATTTTCTACGTTCCAGCTGCATATACCAGCAAGATCGACCCAACG
ACTGGGTTCGTAAATATTTTTAAATTCAAGGATTTGACTGTCGACGCCAAAAGAGAGTTC
ATAAAAAAGTTCGATTCAATTAGGTACGACAGCGAAAAGAATTTGTTCTGCTTTACTTTT
GACTATAACAATTTCATTACTCAGAACACTGTAATGTCTAAGTCCTCTTGGTCAGTCTAT
ACTTATGGCGTTCGTATCAAACGTAGATTTGTTAACGGTAGATTCTCAAATGAAAGTGAT
ACAATAGATATCACGAAAGATATGGAGAAAACATTAGAAATGACAGACATAAACTGGAGA
GACGGACATGACTTGAGACAGGACATTATTGACTACGAGATCGTGCAGCACATCTTTGAG
ATCTTTCGTTTGACCGTACAAATGCGTAACAGTTTATCTGAGCTTGAGGACAGGGACTAC
GATAGATTGATATCACCTGTATTAAATGAGAATAACATCTTCTATGATTCCGCAAAAGCA
GGCGACGCTCTACCCAAAGACGCTGATGCGAACGGTGCTTATTGCATAGCTTTAAAGGGT
TTGTATGAGATCAAACAGATAACAGAAAATTGGAAGGAAGATGGTAAGTTCTCCCGTGAC
AAGCTTAAAATATCAAATAAGGACTGGTTCGATTTTATACAGAATAAGCGTTATTAAAAC
GTCCGGCAGCGACCAAAAAAGCCGGCCAGGCGAAGAAAAAAAAAGCGTCAGGTAGCGGCG
CAGGCAGCCCGAAAAAGAAACGTAAAGTCGAGGATCCGAAAAAGAAACGTAAGGTTATTC
CGGGCTAA
SEQ ID NO: 55
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAATGGA
ACTAATAACTTCCAGAATTTCATTGGTATCTCCTCTTTACAAAAAACTCTAAGAAACGCC
CTAATTCCGACTGAAACTACACAGCAATTCATCGTCAAAAACGGGATCATTAAGGAGGAT
GAGTTGAGGGGTGAAAATCGTCAAATTCTTAAAGACATCATGGACGACTACTACAGGGGG
TTCATCAGCGAGACGTTATCTAGTATAGACGATATAGACTGGACTTCACTGTTCGAGAAG
ATGGAAATCCAATTAAAAAATGGGGACAATAAAGATACACTTATAAAGGAACAGACAGAG
TATAGAAAGGCAATACACAAAAAGTTTGCCAACGACGATCGTTTCAAGAACATGTTTAGT
GCTAAATTGATTTCAGATATTCTGCCGGAATTTGTTATTCACAACAATAATTATAGCGCC
AGTGAGAAAGAAGAAAAAACGCAGGTTATCAAACTGTTCAGTCGTTTCGCTACATCTTTT
AAGGATTACTTTAAAAACCGTGCAAATTGTTTTTCAGCCGACGATATTAGTAGCAGCTCT
TGTCACCGTATTGTTAATGATAATGCGGAGATTTTCTTTTCAAACGCATTGGTCTACAGG
AGGATAGTCAAGTCCCTTTCAAATGACGACATTAATAAGATCTCAGGTGACATGAAAGAT
TCCTTAAAGGAAATGTCCCTGGAAGAGATCTATTCCTATGAAAAGTACGGTGAGTTCATT
ACTCAAGAGGGTATAAGCTTTTACAATGACATATGTGGTAAGGTTAATAGCTTTATGAAC
CTGTATTGCCAGAAGAACAAAGAAAATAAGAATCTGTATAAGTTGCAAAAGCTACACAAA
CAAATTTTGTGCATTGCCGATACATCATACGAGGTGCCATACAAATTCGAGAGCGATGAG
GAGGTTTATCAGAGCGTGAATGGATTCCTGGACAATATTAGTAGTAAGCATATCGTGGAA
AGGCTTAGAAAGATAGGTGACAATTACAATGGCTACAATCTGGATAAAATCTACATCGTC
TCAAAATTCTATGAAAGTGTATCCCAGAAGACGTACCGTGATTGGGAAACTATCAACACC
GCTCTGGAGATACATTACAACAATATACTTCCCGGAAACGGCAAGTCAAAAGCCGACAAA
GTCAAAAAAGCGGTCAAGAACGATTTACAAAAGTCTATCACTGAAATTAATGAATTAGTT
AGTAATTACAAACTGTGTAGTGATGATAATATTAAGGCAGAGACTTACATACACGAAATT
TCACACATTTTAAACAACTTCGAGGCACAGGAACTTAAATATAATCCTGAAATTCACCTG
GTTGAAAGTGAATTGAAAGCCAGCGAGCTAAAGAACGTTTTGGACGTAATCATGAACGCA
TTCCACTGGTGCTCTGTCTTTATGACAGAGGAACTAGTGGATAAGGACAATAATTTTTAT
GCGGAGCTGGAGGAAATATACGATGAGATATATCCCGTAATATCATTATATAATCTGGTA
AGAAACTATGTGACTCAAAAGCCGTATAGCACCAAGAAAATTAAACTTAATTTCGGCATA
CCCACTTTAGCGGACGGCTGGTCAAAATCCAAAGAGTATAGTAATAATGCCATCATCCTG
ATGCGTGACAACCTGTACTATTTAGGTATATTTAACGCCAAAAATAAACCCGACAAAAAG
ATTATAGAGGGCAACACCTCAGAGAACAAAGGTGATTATAAGAAGATGATTTACAACCTT
TTACCCGGTCCTAATAAGATGATTCCCAAAGTCTTTCTATCTAGCAAAACTGGTGTTGAA
ACATACAAACCCTCAGCTTATATTTTAGAAGGGTATAAGCAGAATAAGCATATTAAAAGC
TCCAAAGATTTCGATATTACCTTTTGCCATGACTTGATAGACTATTTCAAAAATTGTATT
GCCATTCACCCTGAATGGAAAAACTTCGGATTTGACTTCTCTGACACATCCACCTACGAA
GACATTTCAGGTTTTTACAGGGAAGTCGAGCTACAGGGTTATAAAATTGATTGGACATAC
ATCAGCGAGAAAGATATTGACCTACTTCAAGAAAAAGGGCAGCTATACCTGTTCCAGATA
TACAATAAAGACTTCAGTAAAAAAAGCACCGGGAACGATAATCTTCACACAATGTACTTA
AAAAATTTATTTAGTGAAGAGAATCTGAAGGATATAGTGCTGAAGTTAAACGGGGAGGCA
GAGATATTTTTTAGAAAATCTAGTATTAAGAATCCGATCATCCACAAGAAGGGTTCTATC
CTTGTTAATAGGACTTATGAGGCAGAAGAAAAAGACCAATTCGGCAACATACAAATTGTC
CGTAAAAATATCCCTGAGAACATTTATCAGGAACTATACAAGTACTTCAATGATAAAAGC
GACAAGGAGCTGAGCGACGAGGCTGCTAAGTTAAAGAATGTGGTGGGCCACCATGAGGCA
GCAACGAATATTGTGAAGGACTATCGTTATACCTACGATAAATACTTTCTTCATATGCCG
ATCACCATTAATTTCAAGGCAAACAAAACTGGCTTCATTAACGATCGTATCTTACAATAT
ATCGCAAAAGAGAAAGACCTTCACGTTATCGGGATCGATAGAGGCGAGCGTAACCTAATT
TATGTTTCTGTGATAGACACCTGTGGGAACATAGTCGAACAGAAATCATTTAATATTGTT
AACGGCTACGATTATCAGATAAAGTTGAAGCAACAAGAGGGTGCACGTCAAATAGCAAGG
AAAGAATGGAAAGAAATAGGCAAGATTAAAGAAATAAAAGAAGGTTATTTATCCCTTGTA
ATACACGAAATTAGCAAAATGGTGATTAAATATAATGCGATCATTGCCATGGAGGATCTT
TCTTACGGCTTCAAAAAGGGGAGATTCAAAGTCGAGAGGCAGGTGTATCAGAAGTTTGAG
ACCATGCTAATCAATAAACTAAATTATCTAGTATTCAAAGACATAAGCATCACCGAAAAT
GGCGGCTTGTTGAAGGGTTATCAATTGACCTACATCCCAGATAAACTAAAAAACGTAGGG
CATCAATGCGGATGTATATTTTACGTTCCAGCCGCATACACTTCCAAAATCGATCCAACT
ACGGGTTTTGTGAACATCTTCAAATTCAAAGACTTGACTGTCGATGCTAAGAGGGAGTTT
ATCAAGAAATTTGACTCCATTAGATACGACAGTGAGAAGAATCTGTTCTGTTTTACCTTT
GATTATAACAACTTTATAACTCAAAACACAGTCATGAGTAAGTCATCTTGGTCAGTGTAT
ACGTATGGTGTGAGGATTAAAAGGAGGTTTGTTAACGGGAGATTTTCCAATGAAAGTGAT
ACAATAGATATAACCAAGGACATGGAAAAGACTCTTGAAATGACCGACATTAACTGGAGA
GATGGCCACGACTTACGTCAAGATATAATCGATTACGAGATAGTGCAACATATCTTTGAG
ATATTTAGGCTTACTGTCCAAATGCGTAACTCATTAAGTGAGTTGGAGGACAGGGATTAC
GATAGGCTAATAAGTCCTGTTCTTAACGAAAACAATATATTCTACGATTCAGCAAAGGCG
GGAGACGCCCTGCCCAAGGACGCGGATGCTAACGGCGCATACTGTATTGCCCTGAAAGGC
TTGTACGAGATAAAACAGATCACGGAGAACTGGAAAGAAGATGGAAAATTCAGTCGTGAC
AAGTTAAAAATTAGTAACAAAGACTGGTTCGACTTTATTCAGAACAAGAGATATCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 56
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAACGGA
ACCAATAACTTTCAAAACTTTATAGGCATCTCCAGTCTACAGAAGACACTACGTAACGCT
TTGATACCAACTGAGACCACGCAGCAGTTTATCGTCAAGAACGGTATTATAAAGGAAGAC
GAGCTAAGGGGGGAAAACCGTCAGATCTTAAAGGACATCATGGATGACTACTACAGAGGC
TTCATAAGTGAGACTTTGTCTAGTATAGACGACATCGACTGGACCAGTTTATTTGAGAAG
ATGGAAATTCAGTTAAAGAACGGGGACAATAAAGACACACTAATTAAAGAGCAGACCGAA
TACAGAAAAGCTATACACAAAAAGTTTGCCAACGATGATAGATTCAAAAATATGTTTTCA
GCAAAATTGATTTCCGACATATTGCCAGAATTCGTAATCCATAATAACAATTATTCTGCA
AGTGAGAAGGAAGAGAAGACCCAAGTAATCAAGCTGTTTTCCCGTTTTGCTACGAGTTTC
AAAGATTATTTCAAGAATAGGGCTAATTGTTTCTCCGCGGACGACATAAGTAGCAGTTCC
TGTCACAGGATTGTGAACGATAATGCTGAGATATTTTTTTCCAATGCCCTAGTGTATAGG
AGAATAGTTAAAAGCTTAAGCAACGACGATATCAATAAAATTTCAGGGGACATGAAGGAC
AGCTTAAAGGAAATGAGTTTGGAGGAGATTTACAGTTATGAAAAATACGGAGAGTTTATA
ACTCAGGAAGGCATCTCTTTCTATAATGATATCTGTGGGAAGGTAAACTCCTTCATGAAT
TTATATTGCCAGAAGAATAAGGAAAACAAAAATCTTTACAAGCTTCAAAAGTTACATAAG
CAGATCTTATGTATTGCCGACACGAGTTATGAAGTGCCTTATAAATTCGAGAGTGATGAG
GAAGTGTATCAGTCTGTTAACGGATTCCTAGATAATATAAGTTCCAAACATATAGTCGAG
AGGCTGAGGAAGATTGGCGATAACTATAATGGATATAATCTTGACAAAATCTATATAGTC
TCTAAATTTTATGAAAGCGTCAGCCAGAAGACATATAGAGATTGGGAAACTATAAACACA
GCCCTTGAAATACATTACAATAACATCCTACCCGGCAATGGTAAGTCTAAGGCAGACAAA
GTTAAAAAAGCAGTAAAGAATGACTTACAGAAGTCAATCACGGAGATAAATGAGTTGGTC
AGTAACTACAAATTATGCTCCGACGATAATATTAAGGCCGAAACATATATACACGAGATA
AGTCATATATTAAACAATTTCGAAGCCCAGGAGTTAAAATATAACCCTGAAATTCATCTG
GTCGAAAGTGAGTTAAAGGCCAGTGAGTTAAAGAATGTACTTGACGTAATTATGAATGCT
TTTCATTGGTGCTCCGTGTTCATGACCGAGGAGTTAGTAGATAAAGACAATAACTTTTAC
GCCGAACTTGAAGAGATATACGACGAGATTTATCCGGTAATCAGCTTGTACAACTTAGTT
AGAAATTATGTAACACAGAAGCCTTACTCTACTAAAAAAATAAAACTGAACTTTGGTATC
CCAACTCTTGCAGATGGTTGGAGTAAAAGCAAGGAATATAGCAACAATGCGATCATCTTG
ATGAGAGACAACTTGTACTATTTGGGAATCTTCAACGCGAAAAATAAACCCGACAAAAAA
ATCATCGAAGGGAATACCTCTGAGAATAAAGGTGACTATAAGAAAATGATTTACAATCTA
CTTCCTGGTCCTAATAAAATGATCCCGAAAGTGTTTCTTAGTTCTAAGACTGGTGTCGAG
ACGTACAAACCTAGCGCGTACATCTTAGAAGGGTACAAGCAGAATAAACACATCAAATCA
AGCAAAGACTTCGATATTACTTTTTGCCATGACTTGATAGACTACTTTAAAAACTGCATA
GCAATCCACCCGGAGTGGAAAAACTTTGGCTTTGATTTCTCTGACACCTCTACATATGAG
GACATATCTGGTTTTTACCGTGAGGTTGAATTGCAGGGATACAAAATTGACTGGACTTAC
ATATCTGAAAAAGATATCGATCTATTGCAGGAGAAAGGCCAGCTTTACCTTTTCCAGATC
TATAATAAGGACTTCTCTAAGAAGTCTACAGGGAATGATAATTTGCACACTATGTACTTA
AAAAATCTGTTTTCCGAGGAAAACTTGAAAGACATTGTTTTAAAGTTGAACGGAGAAGCT
GAAATATTTTTCAGAAAGAGCTCCATAAAAAACCCGATCATTCATAAGAAGGGATCTATC
CTGGTTAACAGAACGTACGAAGCGGAAGAAAAAGACCAATTCGGAAACATTCAAATTGTT
AGAAAGAATATCCCTGAGAACATCTACCAGGAGTTATATAAGTATTTTAATGATAAGTCA
GATAAGGAACTATCTGACGAAGCGGCGAAGCTTAAAAATGTTGTAGGACACCATGAGGCT
GCTACAAATATAGTCAAGGACTACCGTTATACCTACGATAAGTACTTTCTACACATGCCC
ATTACCATCAATTTTAAAGCTAATAAAACGGGTTTTATCAACGATCGTATCCTACAATAT
ATTGCGAAAGAGAAGGATTTGCATGTCATTGGCATTGATAGAGGTGAGAGGAACCTAATA
TACGTATCCGTGATTGATACGTGCGGGAACATAGTTGAACAGAAATCATTTAATATAGTT
AATGGGTACGACTATCAGATTAAGCTAAAGCAACAAGAAGGCGCCAGGCAAATTGCCCGT
AAAGAATGGAAAGAGATCGGGAAGATCAAGGAAATAAAAGAAGGATACCTTTCCCTGGTC
ATCCATGAAATTAGCAAAATGGTGATTAAGTACAATGCCATAATCGCGATGGAGGACTTA
AGCTACGGGTTCAAAAAGGGGAGGTTTAAGGTGGAGAGGCAAGTGTACCAGAAATTTGAG
ACCATGCTAATCAACAAACTGAACTACCTAGTTTTTAAGGACATTTCAATTACAGAGAAT
GGAGGACTTTTAAAGGGTTACCAACTAACGTATATACCAGATAAGTTGAAAAATGTCGGT
CACCAGTGTGGCTGCATCTTTTACGTTCCCGCCGCTTATACATCTAAAATTGATCCAACC
ACAGGCTTTGTAAATATCTTTAAATTCAAAGATTTAACTGTGGATGCAAAAAGAGAGTTT
ATCAAGAAATTCGATAGCATTCGTTATGATAGCGAGAAGAACCTGTTCTGCTTTACTTTC
GACTATAACAACTTTATAACTCAAAACACCGTGATGTCAAAAAGCTCATGGTCAGTCTAC
ACCTATGGTGTAAGGATTAAAAGGCGTTTCGTGAATGGGAGATTCTCCAATGAAAGTGAC
ACGATCGACATAACAAAGGACATGGAGAAGACACTAGAGATGACTGATATTAATTGGAGA
GACGGACACGATCTGCGTCAAGATATAATTGATTATGAGATAGTACAGCACATATTTGAG
ATCTTCCGTTTGACTGTCCAAATGCGTAATTCCCTTTCTGAGCTGGAAGATAGGGACTAT
GATAGATTAATATCCCCTGTACTAAATGAGAACAACATTTTCTATGATAGTGCAAAAGCC
GGGGATGCATTGCCGAAAGACGCTGACGCTAATGGGGCGTACTGTATAGCTTTAAAGGGG
CTTTACGAAATAAAGCAGATAACCGAAAACTGGAAGGAAGATGGCAAATTCTCAAGGGAC
AAACTTAAGATCTCTAACAAGGATTGGTTCGATTTTATACAAAACAAACGTTATTTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 57
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAATGGT
ACAAACAACTTTCAGAATTTCATTGGGATCTCTAGCTTACAGAAGACCCTGAGGAATGCG
TTGATTCCAACTGAAACAACCCAGCAATTCATCGTGAAAAATGGGATAATCAAAGAGGAT
GAGTTAAGGGGTGAAAACCGTCAAATATTGAAGGATATTATGGACGACTACTACCGTGGA
TTCATCTCAGAGACGTTGAGCAGCATTGACGACATAGACTGGACTAGCCTTTTCGAGAAG
ATGGAAATTCAGTTAAAGAACGGAGATAACAAAGATACACTAATCAAGGAACAGACAGAA
TACAGAAAAGCAATTCATAAGAAATTCGCTAATGACGATCGTTTTAAAAACATGTTCTCT
GCAAAATTAATTAGCGACATTCTGCCGGAATTCGTTATACATAATAATAACTACAGTGCT
TCTGAAAAGGAAGAGAAAACTCAGGTAATAAAACTGTTCTCTCGTTTTGCCACATCCTTC
AAAGACTACTTTAAAAATAGAGCGAACTGCTTTAGCGCCGACGATATTAGTTCTTCCTCA
TGCCACAGGATTGTCAACGATAATGCAGAGATATTCTTTTCTAACGCACTAGTCTACAGA
AGGATTGTAAAGTCTTTGTCAAATGATGACATAAACAAGATTAGTGGAGATATGAAAGAC
TCTCTAAAGGAAATGAGCCTTGAGGAGATATACTCTTATGAAAAGTACGGTGAGTTTATT
ACCCAAGAAGGCATTAGTTTCTATAATGACATTTGTGGAAAAGTTAACAGTTTTATGAAT
CTATACTGTCAAAAAAATAAGGAGAATAAAAATCTTTATAAGTTGCAAAAACTGCATAAG
CAGATATTATGTATAGCAGACACGAGCTATGAGGTACCGTACAAGTTCGAGAGCGATGAG
GAAGTCTACCAATCTGTCAACGGATTTTTGGACAACATTTCTTCAAAACATATTGTGGAG
AGGCTTAGGAAAATAGGCGACAATTATAATGGATATAACTTAGATAAGATATATATTGTT
TCCAAATTCTACGAATCTGTAAGCCAGAAGACATACAGAGATTGGGAAACGATAAACACA
GCCCTTGAAATTCACTATAACAACATACTACCTGGAAACGGCAAATCAAAGGCCGACAAA
GTTAAGAAGGCCGTAAAGAATGATTTACAGAAGAGCATAACGGAGATCAATGAGCTGGTG
TCTAACTATAAATTGTGTAGCGATGACAACATAAAAGCCGAGACTTACATTCACGAAATT
TCACACATACTTAACAACTTTGAAGCTCAGGAATTAAAGTATAATCCCGAAATACACCTT
GTGGAGTCCGAACTAAAGGCTAGTGAGCTTAAGAACGTCCTAGACGTAATTATGAATGCC
TTCCACTGGTGTAGTGTTTTTATGACCGAGGAACTTGTTGACAAAGATAATAATTTTTAT
GCAGAACTAGAAGAGATATACGATGAAATATACCCGGTGATCAGTTTGTACAATCTTGTC
AGGAACTATGTGACACAAAAGCCCTATTCAACAAAGAAAATAAAACTTAATTTCGGAATT
CCTACGTTAGCTGATGGCTGGTCTAAATCCAAGGAATACAGCAACAACGCTATAATTCTG
ATGAGAGATAACTTGTACTATCTAGGCATCTTCAATGCCAAAAATAAGCCTGATAAGAAG
ATTATAGAGGGCAACACTTCAGAGAACAAGGGCGACTACAAGAAAATGATCTATAACCTA
TTGCCTGGCCCAAACAAGATGATTCCGAAGGTCTTCCTATCATCCAAGACCGGCGTTGAG
ACATACAAGCCATCAGCGTATATTTTAGAGGGGTACAAACAAAACAAGCACATAAAGTCT
AGTAAAGACTTCGATATAACATTTTGTCATGACTTAATTGACTACTTTAAGAATTGCATC
GCTATACACCCGGAATGGAAGAATTTCGGCTTCGACTTCTCTGATACATCTACCTACGAG
GACATTAGCGGGTTTTACCGTGAAGTCGAATTACAAGGGTATAAGATAGATTGGACGTAC
ATCTCTGAGAAAGACATAGACTTGCTTCAGGAAAAGGGCCAGTTGTATCTATTCCAAATA
TACAATAAGGATTTTTCCAAGAAATCTACGGGTAATGACAATCTTCACACAATGTATCTT
AAGAACCTTTTCTCAGAAGAGAACCTGAAGGACATTGTCTTAAAACTAAATGGCGAAGCT
GAGATTTTTTTCAGGAAGTCTTCAATTAAGAACCCGATAATCCACAAGAAGGGGAGTATT
CTTGTGAATAGAACTTACGAGGCCGAAGAAAAAGACCAATTTGGTAACATCCAGATAGTC
AGAAAGAACATTCCAGAGAACATCTACCAAGAGCTATACAAATATTTCAACGACAAGTCC
GATAAGGAACTGTCCGATGAGGCAGCCAAGTTGAAGAATGTCGTGGGTCATCATGAAGCT
GCTACTAACATTGTCAAGGACTATCGTTATACTTACGACAAGTATTTCCTACACATGCCG
ATAACAATTAATTTCAAGGCTAACAAAACAGGCTTTATCAACGATCGTATCTTGCAGTAC
ATAGCTAAGGAAAAGGATTTGCATGTGATTGGCATTGATAGAGGGGAGCGTAACTTGATA
TATGTGTCTGTCATAGACACGTGTGGCAACATCGTCGAACAGAAATCATTCAACATAGTA
AACGGCTACGATTACCAAATTAAGCTGAAACAGCAAGAGGGTGCACGTCAAATTGCGCGT
AAAGAGTGGAAAGAAATTGGTAAAATCAAGGAAATTAAAGAAGGCTACTTGTCTCTTGTT
ATACATGAAATTTCCAAGATGGTTATAAAGTATAACGCGATAATTGCTATGGAAGACTTA
TCATACGGGTTTAAAAAGGGGAGGTTCAAGGTAGAGAGGCAGGTCTATCAAAAGTTCGAG
ACGATGTTGATTAATAAACTAAACTATCTAGTGTTCAAAGATATCAGCATTACGGAGAAC
GGGGGGCTACTGAAAGGATATCAACTAACGTACATTCCCGATAAGTTAAAGAACGTTGGT
CATCAATGTGGTTGCATCTTCTACGTGCCTGCTGCCTATACGTCCAAAATAGATCCAACT
ACTGGATTTGTTAACATCTTTAAATTCAAAGATTTAACCGTAGACGCCAAAAGGGAATTT
ATAAAAAAATTTGACAGCATCCGTTACGATAGCGAAAAGAATCTGTTCTGTTTTACTTTC
GACTACAATAATTTCATCACGCAAAATACGGTAATGTCTAAGTCAAGTTGGAGCGTCTAC
ACGTATGGAGTCAGGATCAAGAGGCGTTTCGTAAATGGAAGATTCTCTAATGAGTCAGAT
ACTATAGACATCACGAAAGATATGGAGAAAACCTTGGAGATGACGGATATTAACTGGCGT
GATGGACACGATTTAAGACAGGACATTATTGACTATGAGATTGTGCAACACATCTTCGAA
ATATTCCGTCTAACAGTCCAAATGAGGAATAGCCTAAGTGAATTGGAGGACCGTGATTAC
GATAGGCTTATAAGTCCTGTCCTTAACGAAAACAATATTTTCTATGATAGTGCTAAGGCG
GGGGACGCACTGCCTAAAGACGCAGATGCTAACGGGGCATACTGCATTGCGTTAAAGGGT
CTGTACGAAATCAAGCAGATTACGGAAAACTGGAAAGAGGATGGCAAGTTTAGCAGAGAT
AAGTTGAAGATAAGTAACAAAGATTGGTTTGACTTTATTCAGAATAAAAGGTATTTAAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 58
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAACGGC
ACTAATAATTTCCAGAATTTCATCGGCATTAGCAGCTTACAAAAGACGTTGAGGAATGCC
TTAATACCCACAGAAACTACTCAACAATTTATAGTGAAGAATGGGATAATTAAGGAAGAC
GAGTTGAGAGGTGAAAATAGGCAAATCTTGAAAGACATTATGGATGACTACTACAGGGGC
TTCATTAGTGAAACGTTGTCTTCAATAGATGACATTGATTGGACTTCTTTGTTTGAGAAG
ATGGAAATACAGTTAAAGAACGGCGACAATAAGGATACACTTATCAAAGAGCAAACAGAA
TATAGAAAAGCAATTCACAAAAAGTTTGCTAACGATGATAGGTTCAAGAACATGTTTAGC
GCTAAACTAATATCAGACATCCTTCCCGAGTTCGTTATTCATAACAATAACTATAGTGCA
AGTGAAAAAGAGGAGAAGACACAGGTGATTAAGCTGTTCTCCAGATTCGCGACTTCTTTC
AAAGATTACTTCAAAAACAGAGCCAACTGTTTTTCAGCTGACGATATCTCTAGTAGTAGT
TGTCACCGTATAGTGAACGATAACGCTGAGATCTTCTTTAGCAATGCATTAGTGTATAGA
AGGATAGTTAAGTCTCTAAGCAATGATGATATCAATAAAATTTCCGGAGACATGAAGGAC
TCCCTAAAGGAAATGTCCTTAGAAGAGATCTACTCATATGAGAAATACGGGGAATTTATT
ACGCAGGAAGGGATCTCCTTTTACAATGACATATGCGGGAAGGTCAACTCTTTCATGAAC
TTATACTGCCAAAAGAACAAGGAGAACAAGAATTTATATAAACTTCAGAAACTTCACAAA
CAAATACTGTGCATAGCCGATACCTCATATGAGGTTCCTTACAAATTTGAATCAGATGAA
GAGGTATACCAATCCGTTAACGGCTTTCTTGACAATATTAGCTCAAAGCACATCGTGGAG
AGGTTGAGAAAGATTGGTGATAATTATAATGGCTACAATCTAGATAAGATATATATTGTT
AGCAAGTTCTACGAGTCTGTGTCCCAAAAAACATATAGGGATTGGGAGACAATTAATACT
GCTCTAGAAATCCATTACAACAACATCCTTCCTGGAAATGGCAAGAGTAAGGCCGACAAA
GTCAAGAAAGCAGTGAAAAATGATCTGCAAAAATCAATTACTGAGATAAACGAGCTAGTA
TCTAATTACAAGCTTTGTAGCGACGATAACATTAAGGCAGAAACGTACATACACGAGATT
AGTCACATCTTAAATAATTTTGAAGCCCAAGAACTGAAATATAACCCTGAGATACACCTT
GTTGAATCCGAGTTAAAGGCGTCTGAACTAAAAAACGTGTTAGACGTTATTATGAATGCC
TTCCACTGGTGTAGCGTCTTTATGACTGAGGAGTTGGTTGATAAGGATAATAACTTTTAC
GCTGAATTGGAAGAAATTTATGACGAAATCTATCCTGTTATTTCTCTATATAATTTGGTG
AGAAATTACGTAACGCAAAAGCCCTATAGTACGAAAAAAATAAAACTAAATTTCGGGATC
CCTACCCTAGCCGACGGTTGGTCTAAATCCAAGGAGTACTCAAACAATGCAATAATATTG
ATGAGGGACAACCTGTACTACCTAGGCATATTTAATGCCAAAAATAAGCCCGATAAAAAG
ATTATAGAAGGGAACACGTCAGAAAATAAAGGAGACTATAAGAAAATGATCTACAACCTT
TTGCCCGGCCCCAATAAAATGATCCCGAAGGTCTTCCTAAGTAGCAAGACTGGCGTAGAG
ACCTACAAACCATCTGCATACATTTTGGAGGGGTACAAGCAAAACAAGCACATAAAGAGT
AGTAAGGATTTTGACATTACATTCTGCCATGACTTAATTGACTACTTTAAAAATTGCATC
GCAATTCACCCTGAATGGAAAAATTTTGGATTTGATTTCTCTGATACTTCAACATATGAG
GATATTTCAGGGTTCTACAGGGAGGTCGAACTACAGGGTTACAAAATAGACTGGACGTAT
ATTTCTGAGAAAGATATAGATTTGCTTCAGGAAAAGGGTCAGCTATATCTGTTCCAGATA
TATAATAAGGACTTCTCCAAAAAGAGTACCGGAAATGATAATCTGCACACAATGTACTTA
AAAAACTTGTTCTCTGAGGAGAATCTAAAAGACATCGTACTAAAACTTAACGGGGAGGCC
GAAATTTTTTTTAGGAAGTCCAGCATCAAGAACCCGATTATTCATAAAAAAGGTAGCATT
TTGGTGAACCGTACTTATGAGGCGGAAGAAAAAGACCAATTCGGTAATATTCAAATCGTT
AGAAAGAACATCCCTGAGAACATTTATCAGGAACTATACAAATACTTTAACGACAAATCA
GATAAGGAGCTTTCTGATGAGGCAGCTAAATTGAAAAATGTAGTGGGACATCACGAAGCA
GCCACTAACATAGTGAAGGACTACAGATACACATACGATAAGTACTTCCTGCACATGCCT
ATTACAATTAACTTTAAAGCAAATAAAACAGGGTTTATTAACGACAGAATCTTACAGTAT
ATTGCCAAAGAAAAGGATCTGCATGTGATAGGAATAGACAGAGGAGAAAGAAACCTGATA
TACGTCTCCGTGATTGATACATGTGGGAACATAGTAGAACAGAAGTCCTTTAACATTGTT
AATGGGTACGATTATCAAATTAAATTAAAACAACAAGAAGGAGCACGTCAAATAGCTAGG
AAAGAATGGAAAGAGATAGGAAAAATTAAGGAAATTAAGGAGGGTTACCTGTCCCTTGTA
ATTCATGAAATATCCAAAATGGTAATTAAATATAACGCGATCATCGCGATGGAAGATCTA
AGCTACGGGTTCAAAAAAGGCAGGTTTAAGGTGGAGAGGCAAGTTTACCAAAAGTTCGAG
ACAATGTTGATTAATAAGTTAAACTACTTAGTTTTCAAAGATATCTCCATAACCGAGAAT
GGCGGGCTTTTAAAAGGGTACCAACTAACATATATCCCGGATAAATTGAAGAACGTTGGA
CACCAGTGTGGCTGCATATTTTATGTACCCGCTGCGTATACTTCTAAAATTGACCCGACC
ACCGGGTTTGTAAACATATTCAAGTTTAAGGACCTAACAGTTGACGCCAAACGTGAGTTC
ATCAAGAAGTTCGATAGTATAAGGTATGACTCTGAGAAGAACCTTTTCTGCTTCACGTTT
GACTATAATAATTTCATCACCCAAAATACAGTTATGTCAAAAAGCTCTTGGTCAGTATAT
ACGTATGGCGTAAGGATTAAGCGTAGGTTCGTGAACGGTAGATTTTCCAACGAGTCAGAT
ACTATTGATATTACCAAGGATATGGAGAAGACATTAGAAATGACAGATATAAATTGGAGG
GATGGGCACGATCTAAGGCAAGATATCATTGATTACGAAATTGTTCAGCACATATTCGAG
ATATTCCGTCTTACAGTACAAATGCGTAACAGCTTGTCTGAGTTGGAAGATCGTGACTAT
GACAGGTTGATATCACCGGTCTTGAACGAGAACAATATATTCTACGACAGCGCTAAGGCG
GGAGACGCTCTGCCTAAAGACGCAGATGCCAATGGGGCGTACTGCATTGCCTTAAAAGGC
TTATACGAGATTAAACAGATCACAGAGAACTGGAAAGAGGACGGCAAGTTTTCTAGAGAT
AAATTGAAAATCTCAAACAAAGACTGGTTCGATTTCATCCAAAACAAAAGATACCTTAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 59
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAATGGA
ACTAACAACTTCCAGAACTTTATCGGCATCTCTTCCCTCCAAAAGACACTGAGAAATGCA
CTGATCCCAACCGAAACGACTCAACAATTTATTGTTAAGAACGGCATCATAAAAGAAGAC
GAGCTTCGCGGCGAGAACCGCCAGATACTTAAGGATATTATGGACGATTATTACCGAGGC
TTTATCAGCGAAACTCTTAGCTCTATTGATGATATCGACTGGACCTCCCTCTTCGAAAAA
ATGGAGATACAGCTCAAGAACGGCGATAATAAAGACACCTTGATAAAGGAACAGACTGAG
TACAGGAAAGCGATCCACAAGAAATTCGCGAACGACGACAGGTTTAAAAACATGTTCTCT
GCAAAATTGATATCCGACATCTTGCCGGAATTTGTGATACACAACAATAACTATAGCGCT
TCAGAGAAAGAAGAGAAGACCCAAGTAATCAAGTTGTTCAGCCGCTTCGCAACGTCTTTT
AAAGATTACTTTAAGAACCGGGCCAATTGTTTCTCCGCGGATGATATTAGCTCATCAAGT
TGCCATCGAATTGTCAATGATAATGCGGAGATCTTCTTCAGCAATGCGCTGGTCTACAGA
CGAATCGTAAAAAGTCTTTCAAATGACGACATCAATAAGATTAGTGGAGATATGAAGGAT
TCCCTTAAGGAAATGAGTCTTGAAGAAATATACTCATACGAAAAGTACGGGGAATTTATT
ACCCAGGAGGGGATCTCCTTCTATAACGACATCTGTGGAAAAGTAAACTCATTCATGAAC
CTGTACTGTCAGAAAAACAAAGAAAACAAAAATCTGTATAAACTCCAAAAATTGCACAAG
CAAATATTGTGTATAGCGGACACATCATACGAGGTTCCATATAAGTTCGAAAGTGATGAA
GAAGTCTACCAATCAGTGAATGGGTTTCTGGACAACATTAGTTCCAAGCACATAGTTGAA
CGACTGCGAAAGATTGGTGACAATTACAACGGCTATAATTTGGACAAGATTTATATAGTT
AGCAAATTTTATGAATCCGTATCACAAAAGACTTATAGAGACTGGGAAACAATCAACACG
GCACTTGAGATCCATTATAACAATATTCTTCCAGGGAACGGCAAAAGCAAGGCTGATAAG
GTAAAAAAGGCCGTTAAGAATGATCTTCAAAAATCCATAACGGAGATCAACGAACTTGTA
AGTAACTACAAATTGTGCTCTGACGACAATATAAAGGCTGAAACGTATATTCACGAGATT
AGCCATATCCTGAATAACTTTGAGGCCCAAGAACTCAAGTATAACCCGGAAATACATTTG
GTAGAAAGCGAGCTTAAAGCGAGTGAGCTGAAAAACGTCCTCGATGTGATCATGAATGCT
TTCCACTGGTGTAGTGTCTTTATGACTGAGGAGTTGGTTGATAAAGACAATAATTTCTAC
GCTGAACTGGAAGAAATTTACGACGAAATCTATCCAGTGATCTCCCTCTATAACCTCGTT
CGAAACTACGTGACGCAGAAACCTTATTCTACAAAGAAAATTAAGTTGAACTTCGGCATT
CCTACACTTGCTGACGGATGGTCCAAATCCAAAGAGTACTCAAACAACGCAATCATCCTC
ATGCGGGATAACCTTTATTATTTGGGCATTTTCAACGCCAAAAACAAACCTGATAAAAAG
ATAATTGAAGGCAATACGAGTGAGAACAAGGGCGACTACAAAAAAATGATATATAACTTG
TTGCCAGGCCCCAACAAGATGATTCCTAAAGTTTTTCTGTCTTCTAAGACTGGAGTTGAA
ACTTACAAACCCTCCGCCTACATTCTTGAAGGGTATAAACAGAATAAGCACATAAAGTCC
TCAAAGGATTTCGACATTACGTTTTGCCATGACCTCATCGACTATTTCAAGAACTGTATC
GCCATACATCCGGAGTGGAAGAATTTTGGATTTGATTTCTCCGACACATCTACCTATGAA
GACATAAGCGGTTTCTACCGGGAGGTCGAGCTTCAGGGCTATAAGATAGATTGGACATAC
ATTAGTGAAAAAGATATCGATCTTCTGCAAGAAAAGGGACAACTTTACCTTTTTCAGATT
TATAATAAAGACTTTTCAAAAAAGTCCACAGGGAACGATAATCTGCACACCATGTATCTC
AAGAATCTGTTTAGTGAAGAAAACCTTAAAGACATAGTTTTGAAGCTTAACGGAGAGGCT
GAGATTTTTTTTAGAAAGTCCTCAATTAAAAACCCTATAATACACAAGAAAGGCTCTATT
CTTGTTAACAGGACATATGAAGCCGAGGAGAAAGATCAGTTTGGCAATATCCAGATTGTT
CGCAAGAATATCCCGGAAAATATATATCAGGAGCTGTATAAATACTTTAACGACAAGAGC
GACAAGGAGCTGAGTGACGAGGCCGCGAAGCTTAAGAATGTAGTAGGTCACCACGAAGCA
GCCACCAATATCGTCAAAGACTATAGGTACACGTACGACAAGTACTTTTTGCACATGCCT
ATAACTATAAACTTCAAAGCTAATAAAACTGGGTTTATTAATGACAGGATTCTCCAATAC
ATCGCTAAAGAGAAGGATCTGCATGTAATTGGCATAGACAGAGGTGAGAGAAACTTGATA
TATGTCAGCGTAATAGACACATGTGGCAATATCGTGGAACAGAAGTCTTTTAACATCGTC
AATGGTTACGACTACCAAATTAAGTTGAAACAGCAGGAAGGCGCACGACAGATCGCACGA
AAGGAATGGAAAGAGATAGGCAAAATAAAAGAAATAAAGGAGGGCTATCTCAGTCTCGTT
ATACACGAAATTTCAAAAATGGTTATTAAGTACAATGCAATCATAGCGATGGAGGATCTC
AGTTATGGGTTCAAAAAGGGTCGGTTTAAAGTTGAGCGCCAAGTGTACCAAAAGTTCGAG
ACAATGCTGATTAACAAGCTGAACTACCTCGTCTTCAAAGATATAAGTATTACGGAGAAC
GGTGGCCTTCTTAAAGGCTATCAACTTACTTACATCCCGGACAAGCTCAAAAACGTAGGG
CACCAATGCGGGTGTATTTTCTATGTGCCTGCGGCATATACGTCAAAGATTGACCCAACC
ACAGGATTCGTAAACATATTCAAGTTTAAGGACCTCACCGTTGATGCGAAAAGGGAGTTC
ATTAAAAAATTTGATTCTATTCGATATGATAGTGAGAAAAATCTCTTTTGTTTCACATTT
GACTATAATAATTTTATTACTCAGAATACTGTCATGAGCAAGTCATCTTGGTCAGTGTAC
ACATACGGGGTGCGGATCAAACGCAGGTTCGTCAATGGTCGCTTCTCAAACGAATCAGAC
ACCATTGACATCACAAAGGACATGGAAAAAACCCTTGAGATGACCGACATTAATTGGCGC
GATGGTCATGATCTGCGGCAAGACATCATAGACTACGAAATCGTCCAACACATCTTTGAG
ATCTTTCGCTTGACGGTCCAAATGCGGAACTCCCTGTCCGAGCTCGAGGATAGAGATTAT
GATCGGCTGATATCTCCCGTGCTTAATGAAAATAACATCTTCTACGACTCCGCCAAGGCG
GGTGATGCCCTGCCGAAGGATGCGGATGCTAATGGCGCTTATTGCATTGCTCTTAAGGGG
CTCTATGAGATAAAGCAGATCACGGAAAACTGGAAAGAAGACGGTAAGTTTAGTAGAGAC
AAGCTGAAGATCTCAAATAAAGACTGGTTTGATTTCATACAGAACAAGCGGTACCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 60
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAACAATGGC
ACTAACAATTTTCAGAATTTCATCGGCATTTCAAGTCTGCAAAAAACTCTGAGGAATGCT
TTGATCCCTACTGAAACCACTCAGCAATTTATAGTCAAGAACGGTATAATTAAAGAAGAT
GAACTCAGGGGTGAAAATAGACAAATACTCAAGGACATTATGGATGACTATTATAGAGGC
TTCATCTCAGAGACTCTCTCATCAATAGATGATATCGATTGGACTAGCCTTTTCGAGAAA
ATGGAGATTCAGTTGAAAAATGGTGATAACAAAGATACGTTGATAAAGGAACAGACCGAG
TACAGGAAAGCCATTCATAAGAAATTTGCTAATGACGATAGATTTAAGAATATGTTTAGT
GCAAAACTGATTAGTGACATTCTGCCGGAGTTCGTTATCCATAATAATAACTACTCTGCA
TCCGAAAAGGAGGAAAAGACGCAAGTTATTAAACTGTTCAGCCGCTTCGCCACAAGCTTC
AAGGACTACTTCAAAAATAGAGCCAACTGCTTTTCTGCCGACGATATATCATCATCTTCA
TGCCATCGGATCGTTAACGATAACGCCGAGATATTCTTCAGCAACGCCCTTGTATATCGA
AGAATAGTCAAAAGTCTGAGTAATGATGATATTAATAAAATTAGCGGTGATATGAAAGAC
TCCCTGAAGGAAATGTCACTGGAGGAAATTTATAGTTACGAAAAGTACGGCGAATTCATT
ACTCAAGAAGGCATATCCTTCTATAACGACATTTGCGGAAAGGTCAACTCATTCATGAAC
CTTTATTGCCAGAAGAATAAGGAGAATAAAAATCTTTACAAATTGCAAAAACTTCACAAA
CAAATTCTTTGCATCGCGGATACGTCCTACGAAGTTCCTTACAAATTTGAATCCGATGAG
GAAGTGTATCAGAGTGTCAATGGATTTTTGGATAATATCTCTTCAAAACATATTGTGGAG
AGATTGCGCAAAATAGGTGATAACTACAATGGCTACAACCTGGACAAGATTTATATTGTT
AGCAAGTTCTATGAAAGTGTCAGTCAAAAGACCTACAGAGATTGGGAGACAATCAACACG
GCGCTCGAAATACACTACAATAACATCCTCCCCGGCAATGGGAAGAGTAAAGCCGATAAG
GTTAAAAAAGCTGTTAAGAACGACCTCCAGAAATCCATCACGGAAATAAACGAGCTGGTT
TCCAACTATAAGCTGTGTAGCGATGATAATATTAAGGCTGAGACATATATACATGAGATC
AGCCACATTCTCAACAATTTCGAGGCACAGGAACTCAAATACAATCCCGAGATTCACTTG
GTGGAAAGTGAGTTGAAGGCGTCAGAGCTTAAGAATGTACTTGACGTAATAATGAATGCT
TTTCATTGGTGCTCCGTGTTCATGACTGAGGAACTCGTGGATAAGGATAATAACTTTTAT
GCGGAGTTGGAAGAGATATACGATGAAATATACCCGGTTATCTCACTGTATAATCTGGTC
AGAAATTACGTGACCCAAAAGCCTTATAGTACAAAAAAAATAAAGTTGAACTTCGGTATT
CCGACATTGGCAGATGGTTGGTCCAAAAGCAAAGAATACTCTAATAACGCCATTATATTG
ATGCGAGACAATTTGTATTACCTTGGGATCTTTAACGCGAAAAACAAACCGGATAAGAAG
ATCATCGAAGGTAATACATCTGAGAATAAGGGGGATTACAAGAAGATGATTTATAATCTG
TTGCCGGGGCCAAACAAGATGATTCCGAAGGTCTTTCTGTCATCTAAGACAGGAGTAGAG
ACCTACAAACCTTCTGCGTACATTTTGGAAGGCTACAAACAGAACAAGCATATAAAATCT
AGCAAGGACTTTGATATCACGTTTTGTCATGATCTGATAGATTATTTCAAAAACTGCATC
GCTATACATCCTGAGTGGAAGAATTTCGGCTTTGACTTTTCTGACACCAGCACATACGAA
GACATCTCAGGTTTCTACCGGGAAGTCGAGCTCCAGGGGTACAAGATTGACTGGACATAT
ATAAGTGAAAAAGACATCGACCTCCTCCAAGAGAAGGGCCAACTTTACCTGTTCCAGATC
TATAACAAAGACTTTTCTAAAAAGTCCACGGGTAACGACAACTTGCACACTATGTATCTG
AAAAACTTGTTCTCTGAAGAGAACCTCAAGGACATCGTCCTGAAGCTTAACGGGGAGGCG
GAGATCTTCTTTAGAAAGTCCTCTATCAAAAATCCCATTATCCATAAAAAGGGCTCTATA
CTCGTTAATAGGACATATGAAGCGGAGGAAAAAGATCAATTTGGGAACATCCAGATCGTC
CGGAAAAATATACCTGAGAATATCTATCAAGAGCTGTACAAGTATTTTAATGATAAGTCA
GACAAAGAGCTCAGTGATGAGGCGGCAAAGCTCAAGAACGTGGTGGGGCATCATGAAGCT
GCGACGAACATTGTCAAAGATTATAGATACACTTACGATAAATACTTCCTCCACATGCCG
ATAACGATTAACTTCAAAGCCAATAAGACGGGGTTTATAAATGATCGGATCCTTCAGTAC
ATTGCGAAAGAGAAAGACCTCCATGTGATCGGAATTGACCGAGGAGAAAGGAATCTGATT
TACGTGTCCGTGATTGATACTTGCGGGAATATAGTCGAGCAAAAGAGTTTCAACATAGTC
AACGGGTATGACTATCAGATAAAGCTCAAACAGCAGGAAGGTGCGAGGCAAATTGCGCGC
AAAGAGTGGAAGGAGATAGGCAAGATTAAAGAAATCAAGGAAGGTTATCTCAGCTTGGTG
ATCCATGAAATATCTAAGATGGTTATAAAGTACAATGCCATAATAGCCATGGAGGATCTT
TCCTACGGGTTTAAGAAGGGCCGATTTAAAGTGGAGCGACAAGTTTACCAGAAGTTCGAA
ACCATGTTGATTAACAAACTTAACTATTTGGTGTTCAAGGATATAAGTATAACCGAAAAC
GGCGGTTTGCTTAAGGGTTATCAGCTCACGTATATTCCTGATAAACTTAAAAACGTTGGA
CACCAGTGTGGATGTATCTTCTACGTGCCAGCCGCTTACACTAGTAAGATAGATCCTACC
ACGGGGTTTGTGAATATTTTTAAGTTTAAAGACTTGACAGTCGACGCCAAAAGGGAATTT
ATAAAAAAGTTTGATTCTATCCGCTACGATAGTGAAAAAAATCTCTTTTGCTTTACTTTC
GACTATAACAACTTCATTACGCAGAACACTGTCATGAGTAAGTCCAGCTGGAGCGTCTAC
ACATATGGCGTCCGAATTAAACGACGATTTGTAAACGGGCGGTTTTCAAACGAATCTGAC
ACGATAGACATTACCAAGGATATGGAGAAGACACTTGAGATGACCGACATAAACTGGCGG
GACGGTCACGATCTTCGGCAGGACATAATTGATTACGAAATCGTCCAGCATATATTCGAA
ATATTTCGACTTACAGTGCAAATGCGGAACAGTCTCTCTGAACTGGAAGATCGCGATTAT
GACCGGTTGATTTCTCCGGTCCTCAATGAAAATAACATATTTTATGATAGTGCTAAGGCA
GGTGATGCGTTGCCAAAGGATGCAGACGCTAATGGTGCCTATTGTATCGCGCTCAAGGGA
TTGTACGAGATAAAGCAAATTACGGAGAACTGGAAGGAGGATGGTAAGTTTAGCCGAGAC
AAGTTGAAGATTAGCAATAAAGACTGGTTTGATTTTATCCAAAACAAGAGGTACCTGAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 61
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAACGGA
ACTAATAACTTTCAAAATTTCATAGGTATTTCAAGCTTGCAGAAGACCCTGAGGAATGCC
CTGATTCCAACCGAGACAACGCAGCAGTTCATAGTCAAAAATGGCATTATTAAGGAAGAT
GAGCTGCGGGGGGAAAACCGACAGATACTCAAGGATATTATGGACGACTATTACCGGGGA
TTTATCTCAGAAACGCTGAGCAGTATTGATGACATCGATTGGACCAGTCTTTTCGAGAAA
ATGGAAATTCAACTTAAGAATGGTGACAATAAAGACACTCTCATAAAGGAGCAAACTGAA
TACCGAAAAGCCATACACAAAAAGTTTGCCAACGATGACCGCTTTAAAAACATGTTTTCA
GCTAAGCTCATTAGCGACATTCTCCCCGAGTTTGTGATTCATAACAATAACTATAGCGCA
TCCGAGAAGGAGGAAAAAACCCAAGTTATCAAATTGTTCAGTAGATTCGCTACGAGCTTT
AAAGATTACTTTAAAAACCGGGCTAACTGCTTCAGTGCAGACGATATCAGCTCCTCATCC
TGTCATCGCATCGTCAATGATAATGCTGAGATCTTCTTTTCTAATGCACTGGTTTACCGC
AGGATAGTTAAGTCTCTTAGTAACGACGACATCAACAAGATATCAGGAGATATGAAGGAT
TCCCTTAAAGAAATGAGTCTCGAGGAGATATATTCTTATGAAAAATACGGCGAATTTATT
ACCCAAGAGGGCATTAGTTTCTATAATGACATATGCGGAAAAGTTAATAGTTTTATGAAT
CTCTATTGTCAGAAGAATAAGGAGAATAAGAACCTCTACAAATTGCAGAAGTTGCACAAG
CAAATTCTGTGTATCGCGGACACCTCTTACGAGGTCCCATATAAGTTCGAGAGTGATGAA
GAAGTATACCAGAGCGTTAATGGGTTCCTGGACAACATCTCAAGTAAACACATAGTCGAA
AGGCTCCGAAAGATCGGTGATAACTATAACGGATATAATTTGGATAAAATTTATATAGTT
AGCAAATTTTACGAGAGCGTCAGTCAGAAGACCTACCGGGACTGGGAGACCATAAACACA
GCGCTGGAAATACATTATAACAACATACTGCCTGGGAACGGTAAGTCAAAGGCAGACAAG
GTTAAAAAGGCTGTGAAGAATGACCTGCAAAAATCAATTACAGAAATAAATGAGTTGGTA
AGTAATTACAAACTTTGCAGCGATGATAATATAAAGGCAGAGACGTACATACATGAAATA
TCTCATATCCTCAACAATTTCGAAGCCCAAGAACTGAAGTACAACCCGGAAATTCATCTT
GTAGAGTCTGAGTTGAAGGCCTCCGAATTGAAAAACGTTCTTGACGTAATTATGAATGCC
TTCCACTGGTGCTCAGTATTCATGACGGAAGAGCTCGTGGATAAAGACAACAATTTTTAC
GCTGAACTGGAAGAAATATATGACGAGATTTACCCCGTAATTTCACTCTACAACTTGGTA
CGAAATTACGTTACCCAAAAGCCATACTCAACAAAAAAAATTAAACTGAACTTCGGGATA
CCCACCCTCGCAGATGGATGGTCAAAGTCCAAAGAGTACAGTAACAATGCAATTATCCTG
ATGCGAGACAACCTTTATTACCTCGGGATTTTCAACGCTAAAAATAAACCTGATAAAAAA
ATAATTGAGGGTAATACCTCTGAAAACAAGGGGGATTATAAAAAGATGATATACAATCTG
CTGCCTGGCCCGAACAAAATGATTCCTAAAGTCTTCTTGTCTTCCAAGACTGGAGTCGAA
ACCTACAAGCCAAGTGCTTATATACTCGAAGGGTACAAACAAAATAAGCACATAAAATCC
AGCAAGGATTTTGATATTACATTCTGCCACGATTTGATTGATTATTTTAAGAACTGTATA
GCCATCCACCCAGAATGGAAGAATTTTGGTTTTGATTTTAGCGATACCTCAACATATGAG
GATATCTCTGGCTTTTACCGCGAGGTAGAACTGCAAGGTTATAAGATCGATTGGACTTAT
ATTTCTGAAAAGGACATAGATCTCCTGCAAGAGAAAGGGCAACTTTATTTGTTTCAAATA
TACAACAAAGATTTTAGTAAGAAGAGTACTGGCAATGATAACCTTCACACTATGTATCTG
AAGAACCTTTTTTCTGAGGAGAACTTGAAGGACATAGTCCTTAAACTCAATGGGGAAGCT
GAAATATTCTTTCGCAAAAGCTCCATTAAAAACCCGATCATTCATAAAAAGGGTTCCATC
TTGGTAAACCGCACATACGAGGCGGAAGAAAAAGATCAGTTCGGAAATATCCAGATCGTA
AGGAAGAATATCCCCGAAAATATATACCAAGAGCTTTACAAATATTTTAACGATAAGTCA
GACAAGGAACTGTCAGACGAAGCAGCCAAGTTGAAGAATGTCGTAGGGCACCACGAAGCA
GCTACAAACATAGTTAAAGATTATCGGTACACCTACGATAAATATTTCCTGCATATGCCA
ATAACCATAAACTTCAAAGCCAACAAAACAGGGTTCATCAATGACCGAATACTTCAGTAT
ATAGCCAAGGAAAAAGACCTGCATGTTATAGGAATAGATAGAGGTGAGCGCAACTTGATA
TATGTCAGCGTGATAGACACCTGCGGAAATATCGTCGAGCAAAAAAGTTTCAACATTGTT
AATGGCTACGATTACCAAATTAAATTGAAGCAGCAAGAGGGGGCTCGGCAAATCGCGCGA
AAGGAATGGAAAGAAATCGGGAAGATTAAAGAAATTAAAGAGGGCTACCTGTCTCTTGTA
ATTCACGAAATATCTAAGATGGTCATCAAGTATAATGCCATTATTGCGATGGAAGATCTG
TCCTACGGATTTAAGAAAGGCAGGTTTAAAGTCGAAAGGCAGGTGTACCAGAAATTCGAG
ACCATGCTGATTAATAAGCTCAACTATCTCGTATTTAAGGATATTTCTATAACTGAAAAT
GGAGGGCTTCTCAAAGGATATCAACTCACATACATACCTGATAAGCTGAAGAACGTAGGC
CACCAGTGTGGATGCATATTCTATGTACCAGCTGCATACACAAGCAAGATCGATCCAACT
ACTGGGTTTGTCAATATCTTCAAATTTAAGGACTTGACGGTCGATGCCAAACGGGAGTTC
ATCAAAAAGTTTGATAGTATTCGATATGATAGTGAGAAGAACTTGTTTTGCTTCACATTT
GACTACAACAATTTCATAACGCAAAATACGGTTATGTCTAAATCCTCATGGAGCGTCTAC
ACTTACGGAGTGAGGATAAAGCGGCGCTTCGTAAATGGCAGGTTTAGCAATGAATCCGAC
ACGATTGACATAACCAAGGATATGGAGAAAACCCTCGAGATGACCGATATAAATTGGCGG
GATGGACACGATCTGCGACAAGACATAATCGATTATGAAATCGTGCAGCACATATTTGAG
ATATTCAGGCTTACGGTCCAAATGAGAAATTCCCTTTCCGAACTTGAAGACCGCGATTAC
GACCGACTGATAAGCCCCGTTCTGAACGAAAATAACATCTTCTACGACAGCGCTAAAGCG
GGAGACGCGCTGCCGAAAGATGCGGACGCAAATGGAGCCTATTGTATCGCCTTGAAAGGG
TTGTACGAGATCAAACAGATAACCGAGAATTGGAAGGAGGATGGGAAGTTTAGTCGAGAC
AAACTTAAAATAAGCAACAAGGACTGGTTCGACTTTATTCAAAACAAACGATATCTCAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 62
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAATGGT
ACTAACAATTTTCAAAACTTTATCGGCATCTCTTCACTTCAGAAAACTCTTCGGAACGCC
CTTATACCGACGGAGACAACGCAGCAGTTTATAGTTAAAAACGGGATCATTAAAGAAGAT
GAACTCAGAGGGGAAAACAGGCAAATATTGAAGGACATTATGGACGATTACTACCGGGGG
TTTATTTCAGAGACCCTTTCATCTATTGATGACATAGATTGGACCTCCCTTTTCGAGAAA
ATGGAGATACAATTGAAAAACGGCGACAATAAAGATACACTTATCAAGGAACAAACTGAG
TATCGCAAGGCGATTCACAAGAAGTTTGCGAATGACGATCGCTTTAAGAATATGTTTTCT
GCGAAGCTCATAAGTGACATTCTGCCTGAATTTGTCATTCATAACAACAATTATTCTGCT
AGCGAAAAAGAGGAAAAAACTCAAGTCATTAAGCTTTTTAGCAGGTTCGCTACTAGTTTT
AAAGACTATTTTAAGAACCGGGCGAATTGCTTTAGCGCTGACGACATATCATCCTCATCC
TGTCATCGCATAGTCAATGATAATGCAGAAATATTCTTTTCTAATGCGCTCGTGTATCGG
AGAATAGTGAAAAGCCTCTCTAACGATGACATTAACAAAATAAGCGGCGATATGAAGGAT
AGTCTGAAGGAAATGTCCCTCGAAGAAATATACTCATACGAGAAGTACGGAGAATTTATC
ACCCAGGAAGGAATTAGTTTTTACAACGACATCTGTGGTAAGGTTAACTCTTTTATGAAT
CTGTATTGTCAAAAGAATAAAGAAAATAAAAATCTTTATAAGCTCCAAAAGCTTCACAAA
CAAATCTTGTGCATTGCGGATACGTCATACGAAGTACCTTACAAATTTGAAAGCGACGAA
GAGGTGTATCAGTCAGTGAATGGGTTCCTTGACAATATTTCTAGCAAACATATTGTGGAG
CGACTTCGAAAGATCGGTGATAATTACAATGGCTATAATTTGGATAAAATTTACATAGTT
AGTAAGTTTTATGAATCCGTCTCACAAAAGACGTACCGAGATTGGGAGACCATCAACACT
GCTCTGGAGATTCATTACAATAATATATTGCCTGGGAATGGGAAGTCAAAGGCCGACAAG
GTTAAAAAAGCCGTAAAAAACGATCTTCAAAAGTCCATTACCGAGATAAATGAACTTGTA
TCCAACTATAAGTTGTGCTCTGACGATAATATTAAAGCAGAAACGTATATCCACGAAATA
AGTCACATCCTGAACAACTTCGAAGCTCAAGAGCTCAAGTATAATCCTGAAATTCATCTC
GTCGAAAGCGAGCTGAAAGCATCCGAGTTGAAGAATGTGCTTGATGTGATCATGAACGCA
TTCCATTGGTGCAGTGTGTTCATGACCGAAGAACTTGTAGACAAAGACAACAACTTCTAC
GCTGAATTGGAAGAGATTTACGATGAAATTTACCCCGTGATATCCCTCTATAATCTGGTA
AGAAATTACGTCACGCAAAAACCATACAGTACCAAGAAAATAAAGCTCAACTTTGGTATT
CCGACGTTGGCAGATGGGTGGAGTAAGAGCAAGGAGTATTCTAACAATGCAATCATCCTC
ATGCGCGACAATTTGTATTATCTGGGGATCTTCAACGCGAAAAATAAGCCCGACAAAAAG
ATAATAGAAGGCAATACGTCCGAGAACAAAGGGGACTATAAGAAAATGATTTATAACCTT
CTTCCAGGACCCAACAAGATGATCCCAAAGGTTTTCTTGAGTTCAAAAACCGGCGTAGAA
ACTTATAAACCGTCCGCCTACATTCTGGAAGGGTACAAGCAAAACAAGCACATTAAGTCA
TCTAAGGATTTCGACATTACTTTTTGTCATGATTTGATAGACTACTTCAAAAATTGTATA
GCGATACATCCGGAATGGAAAAATTTTGGGTTCGATTTTTCCGACACAAGTACTTATGAA
GACATCTCAGGGTTTTATAGGGAAGTTGAACTGCAAGGTTACAAAATAGACTGGACTTAT
ATTAGTGAGAAGGACATTGATTTGCTCCAGGAAAAGGGTCAATTGTATCTGTTCCAGATA
TATAACAAGGATTTCTCTAAAAAATCTACAGGTAACGACAATCTCCACACGATGTACCTC
AAGAATCTCTTCAGCGAAGAGAATTTGAAGGATATCGTACTTAAGCTCAATGGAGAAGCG
GAAATATTCTTCAGAAAGTCCAGCATTAAGAATCCTATAATTCACAAGAAAGGGTCAATT
CTCGTAAACCGGACTTATGAGGCCGAAGAAAAAGATCAGTTTGGTAACATTCAGATTGTA
CGGAAAAACATTCCCGAGAACATCTATCAAGAACTGTATAAATACTTTAATGATAAATCC
GACAAGGAACTTTCTGACGAGGCTGCAAAATTGAAGAACGTAGTGGGACACCATGAGGCC
GCAACCAATATAGTAAAGGATTACAGATACACTTATGATAAGTATTTCCTCCATATGCCG
ATCACGATTAATTTCAAGGCGAATAAAACCGGCTTCATTAACGATCGCATTTTGCAATAT
ATTGCGAAGGAAAAGGATTTGCACGTGATAGGTATAGACCGGGGTGAACGAAACTTGATT
TACGTCTCTGTGATCGACACATGCGGAAATATAGTTGAACAGAAGTCCTTTAATATTGTG
AATGGTTACGACTACCAGATAAAATTGAAGCAACAGGAGGGCGCAAGACAGATAGCTCGC
AAAGAGTGGAAGGAAATCGGCAAGATCAAAGAAATAAAGGAGGGTTATCTTTCCCTGGTA
ATTCATGAAATTAGCAAGATGGTTATTAAGTATAATGCTATAATAGCTATGGAGGACCTT
TCCTATGGGTTCAAGAAAGGTCGCTTCAAAGTGGAGCGACAAGTGTATCAAAAGTTCGAG
ACTATGTTGATAAATAAATTGAATTATTTGGTTTTTAAAGACATTTCAATAACTGAGAAC
GGGGGTCTCTTGAAGGGGTACCAATTGACTTATATTCCGGACAAGTTGAAGAATGTCGGA
CACCAGTGTGGTTGCATTTTCTACGTGCCTGCCGCTTACACCTCAAAAATCGATCCGACC
ACTGGTTTTGTAAATATATTTAAATTCAAAGATCTCACCGTTGATGCCAAACGGGAGTTT
ATCAAAAAATTCGATTCCATTCGCTACGACTCTGAGAAAAACCTTTTTTGTTTCACGTTC
GATTATAACAACTTTATAACCCAAAATACTGTAATGTCCAAGTCAAGTTGGTCTGTCTAT
ACTTACGGAGTAAGGATCAAGCGCCGCTTCGTTAATGGGAGATTCTCAAACGAGTCTGAT
ACCATAGACATAACTAAAGACATGGAAAAAACCCTGGAAATGACGGACATCAATTGGCGA
GACGGGCATGATCTTCGACAGGACATAATAGATTACGAAATTGTTCAACACATTTTCGAG
ATATTTCGACTTACGGTTCAGATGAGGAATTCCCTTTCCGAATTGGAAGACCGGGATTAT
GATCGACTTATATCTCCCGTGCTCAATGAAAACAATATTTTTTATGATTCAGCGAAAGCT
GGGGACGCGCTGCCAAAAGATGCCGATGCCAATGGAGCATACTGTATCGCCCTGAAGGGT
TTGTATGAGATTAAGCAAATTACTGAAAACTGGAAGGAAGATGGCAAGTTTTCTAGAGAT
AAGCTTAAGATTAGCAATAAGGACTGGTTTGACTTCATTCAAAATAAAAGGTATCTTAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 63
ATGGGCCATCATCATCATCATCACAGCAGCGGCGTCGATCTGGGTACCGAGAATTTGTAT
TTCCAGAGCCCAGCGGCTAAAAAAAAGAAACTGGATGGCAGCGTGGATATGAATAATGGA
ACAAATAATTTTCAAAATTTTATTGGTATCAGTTCATTGCAAAAGACTTTGAGAAATGCT
TTGATCCCGACTGAGACCACACAGCAGTTCATCGTCAAAAATGGCATAATCAAGGAAGAC
GAACTTAGGGGTGAGAATAGACAAATATTGAAGGACATCATGGATGACTATTATAGGGGG
TTCATTTCCGAAACGCTCAGTAGTATTGATGACATTGACTGGACTAGTCTTTTCGAGAAA
ATGGAAATTCAGCTTAAGAACGGGGACAATAAAGACACGCTGATCAAGGAGCAAACGGAA
TATAGGAAGGCGATCCATAAAAAATTCGCGAATGATGATCGGTTTAAAAACATGTTTAGT
GCCAAGTTGATCAGCGACATACTGCCCGAATTCGTGATCCACAACAATAATTACAGCGCC
TCCGAAAAGGAGGAAAAAACTCAGGTCATTAAATTGTTTAGCCGATTCGCAACGAGTTTC
AAAGATTATTTTAAGAACCGGGCCAACTGTTTTTCAGCGGATGATATTAGCTCCAGCAGC
TGCCATCGCATAGTAAATGATAACGCTGAAATCTTTTTTAGCAACGCACTTGTCTACCGG
AGGATTGTAAAATCACTGTCAAATGATGACATTAACAAAATATCTGGAGATATGAAGGAC
TCACTCAAAGAAATGAGCCTGGAAGAAATATATTCATACGAAAAATACGGGGAGTTTATT
ACCCAGGAAGGTATCAGTTTTTATAATGATATATGTGGAAAAGTTAATTCATTTATGAAT
CTTTACTGTCAAAAAAATAAGGAGAACAAGAATTTGTACAAGCTCCAAAAACTTCATAAA
CAGATTCTGTGCATCGCAGACACAAGTTATGAGGTACCGTACAAATTTGAGAGCGACGAA
GAAGTTTATCAGAGTGTGAATGGTTTCCTGGACAATATCTCTTCTAAACACATTGTTGAG
AGGCTTAGGAAGATCGGTGATAATTATAACGGCTATAATCTGGACAAAATTTATATTGTA
TCAAAGTTTTATGAATCAGTCTCTCAAAAGACGTATCGGGATTGGGAAACAATTAACACG
GCTCTGGAGATCCACTACAATAACATTCTGCCCGGCAACGGGAAGAGCAAAGCTGATAAG
GTCAAGAAGGCAGTCAAGAACGACCTTCAGAAGAGCATAACAGAAATTAACGAATTGGTC
AGTAACTACAAACTGTGTAGTGATGACAACATAAAAGCCGAAACATACATCCATGAAATA
AGCCATATCCTGAATAACTTCGAAGCCCAAGAACTTAAATACAATCCCGAGATTCATCTT
GTCGAATCAGAACTCAAGGCGTCCGAGCTCAAAAATGTCCTTGACGTGATAATGAATGCC
TTCCACTGGTGCAGCGTATTCATGACGGAGGAGTTGGTAGATAAAGACAACAACTTTTAT
GCCGAATTGGAAGAGATTTATGATGAGATTTACCCCGTTATTTCTCTGTACAACTTGGTT
CGAAACTACGTAACACAAAAACCATACTCAACCAAAAAGATCAAACTCAATTTTGGCATA
CCTACATTGGCTGATGGTTGGTCCAAGTCAAAGGAATATAGCAATAATGCAATAATTCTC
ATGCGAGATAACTTGTATTATTTGGGGATCTTTAACGCTAAGAACAAACCAGATAAAAAG
ATAATCGAGGGGAACACAAGTGAGAACAAGGGTGATTACAAAAAAATGATTTACAATCTG
CTTCCTGGGCCTAACAAAATGATTCCGAAGGTGTTTCTTAGCTCTAAAACTGGAGTGGAG
ACGTATAAGCCTTCCGCGTACATTCTCGAAGGCTACAAGCAAAATAAGCATATCAAGTCC
AGTAAGGACTTCGACATCACTTTTTGCCACGATCTCATCGATTACTTTAAGAACTGTATC
GCAATACACCCCGAGTGGAAAAACTTTGGTTTTGATTTTTCAGACACTAGTACCTACGAG
GACATTTCCGGCTTCTATCGAGAAGTCGAACTCCAGGGCTACAAAATCGATTGGACGTAC
ATTTCTGAGAAGGACATCGACTTGCTCCAAGAGAAAGGTCAACTTTACCTCTTCCAAATT
TACAATAAAGACTTTTCAAAGAAGAGCACCGGTAATGACAACTTGCATACCATGTATCTG
AAGAACCTGTTTTCTGAGGAGAACCTCAAGGATATTGTATTGAAGTTGAATGGCGAAGCA
GAAATATTTTTCCGAAAGTCATCTATCAAGAACCCCATTATACACAAAAAAGGCTCTATC
CTGGTGAACCGGACTTACGAGGCAGAGGAGAAGGATCAATTCGGAAACATACAGATAGTC
CGCAAAAACATCCCTGAGAATATCTATCAGGAACTCTATAAGTACTTCAATGATAAATCA
GACAAGGAGCTTAGCGACGAAGCAGCTAAACTTAAAAACGTGGTTGGCCATCACGAGGCC
GCTACCAACATAGTCAAAGACTACCGCTATACTTATGACAAGTACTTTTTGCACATGCCC
ATAACAATTAATTTCAAAGCTAACAAAACAGGGTTTATAAATGACAGAATCCTCCAATAC
ATCGCCAAAGAGAAGGACCTCCATGTAATCGGGATTGATAGAGGCGAACGGAACTTGATT
TACGTTAGTGTCATTGATACCTGTGGTAACATTGTCGAACAAAAGTCATTCAACATAGTC
AATGGATATGATTATCAGATAAAACTCAAGCAACAAGAAGGCGCGAGGCAGATTGCCAGG
AAGGAATGGAAAGAAATCGGGAAGATCAAGGAGATCAAGGAGGGTTACCTGTCCTTGGTG
ATACACGAGATTTCAAAAATGGTTATAAAATACAATGCCATTATCGCGATGGAGGATTTG
TCTTATGGATTTAAGAAGGGGAGGTTCAAAGTCGAACGACAAGTCTATCAGAAGTTTGAA
ACAATGCTCATTAACAAGCTCAATTACCTTGTTTTCAAGGATATAAGCATCACTGAAAAC
GGCGGACTCCTTAAGGGATATCAGCTGACTTATATCCCCGACAAGCTCAAGAACGTAGGG
CACCAATGCGGATGCATCTTTTACGTGCCTGCAGCATATACTTCAAAAATTGATCCGACT
ACTGGCTTTGTTAACATTTTCAAGTTCAAGGATCTGACGGTAGACGCTAAGAGAGAATTC
ATAAAAAAGTTTGACAGCATCAGGTACGATAGTGAAAAGAACCTTTTTTGTTTTACCTTT
GACTACAATAATTTTATTACGCAAAATACAGTTATGAGCAAATCAAGTTGGAGCGTTTAC
ACATATGGCGTTCGGATCAAGCGCAGATTCGTCAATGGTCGCTTCTCAAATGAGAGCGAT
ACAATCGATATAACGAAGGATATGGAGAAGACGCTTGAGATGACAGATATCAACTGGCGG
GACGGACATGACCTTAGACAAGACATAATCGATTACGAAATAGTACAGCATATCTTTGAG
ATTTTTAGGCTTACAGTTCAGATGCGGAACTCTCTTTCCGAACTGGAGGACCGGGATTAT
GATCGGTTGATCTCCCCAGTACTGAACGAAAATAATATCTTTTACGATAGCGCGAAGGCT
GGTGATGCACTCCCAAAAGACGCTGATGCGAACGGAGCTTATTGCATAGCCCTTAAAGGG
CTTTACGAGATTAAACAAATAACAGAAAATTGGAAGGAAGATGGCAAATTTTCCCGCGAC
AAGTTGAAGATTAGTAACAAAGACTGGTTCGACTTCATTCAGAATAAACGCTACCTCAAA
CGTCCGGCAGCGACCAAAAAAGCCGGCCAGGCGAAGAAAAAAAAAGCGTCAGGTAGCGGC
GCAGGCAGCCCGAAAAAGAAACGTAAAGTCGAGGATCCGAAAAAGAAACGTAAGGTTATT
CCGGGCTAA
SEQ ID NO: 64
AGCCCAGCGGCTAAAAAAAAGAAACTGGATGGCAGCGTGGATATGAATAACGGTACCAAT
AACTTCCAGAACTTCATCGGTATTTCTAGCCTGCAAAAGACCCTGCGTAACGCGCTGATT
CCGACCGAGACTACCCAGCAATTCATCGTGAAAAACGGTATCATTAAGGAAGATGAATTG
CGCGGTGAGAATCGTCAGATTCTGAAAGATATCATGGATGACTACTATCGCGGTTTCATT
AGCGAAACCCTGTCGAGCATCGATGATATCGATTGGACGAGCCTCTTCGAGAAAATGGAA
ATTCAACTGAAAAATGGTGACAACAAAGATACCCTGATTAAAGAACAAACGGAATACCGC
AAGGCAATCCATAAAAAGTTTGCGAATGACGACCGTTTTAAGAATATGTTCTCGGCCAAG
CTGATTTCCGACATCCTGCCAGAGTTCGTCATTCACAACAACAATTACAGCGCAAGCGAG
AAAGAGGAAAAGACTCAGGTCATTAAGCTGTTTAGCCGCTTTGCGACGTCCTTCAAAGAC
TACTTCAAGAATCGTGCGAATTGCTTTAGCGCGGATGACATCTCTAGCTCTAGCTGTCAC
CGTATTGTTAACGACAATGCAGAGATTTTCTTCAGCAACGCCCTGGTGTATCGCCGTATT
GTCAAGTCTCTGAGCAACGACGACATTAACAAGATCAGCGGCGACATGAAAGACAGCCTG
AAAGAAATGTCTCTGGAAGAAATCTACAGCTACGAGAAATATGGTGAGTTTATCACCCAA
GAGGGCATTAGCTTCTACAATGATATCTGTGGTAAGGTTAATAGCTTTATGAATCTGTAC
TGCCAGAAGAATAAAGAAAACAAGAACTTGTACAAGCTGCAAAAGCTGCATAAGCAAATT
CTGTGCATCGCCGATACTAGCTATGAAGTTCCGTACAAGTTCGAGTCTGATGAAGAGGTG
TATCAGTCAGTCAACGGTTTTCTGGATAACATCAGCAGCAAGCACATCGTCGAGCGCCTG
CGCAAGATTGGTGACAACTACAATGGTTATAACCTGGACAAGATCTATATCGTGTCGAAG
TTTTACGAGAGCGTGTCCCAGAAAACGTACCGTGATTGGGAAACGATTAACACGGCCTTG
GAAATTCACTATAACAATATCCTGCCGGGCAACGGCAAGAGCAAAGCTGACAAAGTCAAA
AAAGCTGTGAAAAACGATCTGCAAAAGTCCATCACCGAGATCAACGAACTGGTTAGCAAC
TATAAGCTGTGTAGCGACGACAACATTAAAGCTGAAACGTATATCCACGAAATCAGCCAC
ATCCTGAATAACTTTGAGGCACAAGAACTGAAATACAATCCTGAGATCCATCTGGTAGAG
AGCGAGCTGAAGGCAAGCGAGTTGAAAAACGTTCTCGACGTTATCATGAATGCTTTCCAC
TGGTGTAGCGTGTTTATGACCGAAGAACTGGTTGACAAAGATAACAATTTCTATGCAGAG
CTGGAAGAAATCTATGATGAAATCTACCCGGTCATCAGCCTGTATAACCTGGTTCGTAAC
TACGTGACGCAGAAGCCGTACAGCACCAAAAAGATCAAGCTGAACTTCGGTATTCCGACC
TTGGCGGACGGTTGGAGCAAATCCAAAGAATACTCCAATAATGCGATTATTCTGATGCGT
GATAATCTGTACTATCTGGGTATCTTCAATGCGAAGAACAAGCCAGATAAAAAGATTATT
GAAGGCAACACCAGCGAGAATAAAGGCGACTACAAGAAAATGATCTACAACTTATTGCCG
GGTCCGAACAAGATGATCCCGAAAGTTTTTCTGAGCAGCAAGACCGGCGTTGAAACCTAT
AAGCCGAGCGCGTACATTTTAGAGGGCTATAAACAAAACAAGCACATCAAGAGCAGCAAA
GATTTTGATATTACGTTCTGCCACGACCTGATCGACTATTTCAAGAATTGTATTGCGATT
CACCCTGAGTGGAAGAACTTCGGTTTTGACTTTTCCGATACCTCCACCTATGAAGATATT
AGCGGTTTTTACCGTGAAGTCGAGTTGCAGGGTTATAAGATTGATTGGACTTACATTTCC
GAGAAAGACATCGACCTGTTGCAAGAGAAAGGTCAGCTGTACCTGTTTCAGATCTATAAC
AAAGATTTCAGCAAAAAGTCGACGGGCAATGATAATCTGCACACCATGTATCTGAAAAAC
CTGTTTAGCGAAGAGAACCTGAAAGACATTGTTCTTAAGCTGAATGGTGAGGCCGAGATC
TTCTTCCGTAAAAGCTCCATTAAGAACCCGATTATCCACAAAAAGGGCTCTATTCTGGTT
AACCGCACGTACGAAGCGGAAGAGAAAGATCAATTTGGTAACATCCAGATCGTGCGTAAG
AATATCCCGGAGAACATTTACCAAGAACTGTATAAGTATTTCAATGACAAGAGCGATAAA
GAATTGAGCGATGAAGCGGCAAAGCTGAAAAACGTCGTTGGCCACCACGAAGCCGCGACG
AATATCGTGAAAGATTATCGTTACACCTACGACAAGTACTTTCTGCACATGCCGATCACC
ATCAATTTCAAAGCGAATAAAACGGGTTTTATCAATGACCGTATCCTGCAGTACATTGCG
AAAGAAAAAGATTTACACGTGATTGGTATTGATCGCGGCGAGCGCAATCTGATTTACGTC
AGCGTTATCGACACGTGCGGCAATATTGTGGAGCAGAAAAGCTTCAATATCGTCAATGGT
TACGACTACCAGATCAAACTGAAGCAACAAGAGGGCGCCCGCCAGATTGCGCGTAAAGAG
TGGAAAGAAATCGGTAAGATTAAAGAAATCAAGGAAGGCTACCTGTCCCTGGTGATCCAT
GAAATCAGCAAAATGGTGATCAAGTACAACGCTATCATTGCGATGGAAGATCTGAGCTAC
GGTTTTAAAAAGGGTCGCTTCAAAGTTGAGCGTCAAGTGTATCAGAAATTTGAGACTATG
CTGATTAACAAGTTGAACTATCTGGTTTTTAAAGACATCAGCATTACCGAGAATGGTGGC
CTGCTGAAGGGTTATCAACTGACCTATATTCCTGACAAGTTGAAAAATGTTGGTCATCAG
TGTGGTTGCATTTTCTACGTACCGGCAGCGTACACGAGCAAGATTGACCCGACCACGGGT
TTCGTTAACATTTTCAAGTTTAAAGATTTGACCGTGGACGCCAAGCGTGAGTTCATTAAA
AAGTTCGACAGCATCAGATACGACTCTGAGAAGAATCTGTTCTGCTTTACGTTCGACTAC
AATAACTTCATTACCCAAAATACCGTTATGAGCAAAAGCTCCTGGAGCGTGTACACGTAC
GGCGTCCGTATCAAGCGTCGTTTTGTGAATGGTCGCTTTTCCAACGAATCTGACACCATT
GACATTACCAAAGATATGGAAAAGACCCTTGAGATGACCGACATTAATTGGCGTGATGGC
CATGACTTGCGCCAAGACATTATCGACTACGAAATTGTTCAGCACATCTTTGAGATTTTT
CGTCTGACGGTCCAGATGCGCAACTCGCTGAGCGAGTTGGAAGATCGTGACTATGACCGT
CTGATTAGCCCGGTGCTGAATGAAAACAATATCTTCTATGATAGCGCAAAGGCCGGTGAC
GCGCTGCCGAAAGATGCGGATGCTAACGGTGCATACTGCATTGCACTGAAGGGTCTGTAC
GAAATCAAACAGATCACCGAGAATTGGAAAGAGGATGGTAAGTTTAGCCGTGATAAGCTG
AAGATTAGCAATAAAGACTGGTTCGACTTTATTCAAAACAAGCGCTATCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 65
AGCCCAGCGGCTAAAAAAAAGAAACTGGATGGCAGCGTGGATATGAACAACGGAACAAAT
AATTTTCAGAACTTTATTGGGATCAGTTCGCTTCAGAAAACGCTTCGTAATGCTCTGATT
CCCACAGAAACCACTCAGCAGTTTATCGTAAAGAATGGCATTATCAAGGAGGATGAATTA
CGCGGCGAGAACCGCCAAATCTTAAAAGATATCATGGACGACTACTACCGCGGTTTCATT
AGCGAAACTCTTAGTTCAATTGACGACATTGACTGGACGTCCTTGTTCGAAAAGATGGAG
ATTCAATTAAAGAACGGTGATAACAAGGATACGTTGATTAAAGAACAGACGGAGTACCGT
AAGGCTATCCACAAAAAATTTGCAAACGACGACCGCTTTAAAAATATGTTTAGCGCAAAA
TTAATCTCCGACATCCTGCCTGAATTCGTCATCCATAACAATAACTATAGCGCCTCGGAA
AAAGAAGAAAAAACGCAGGTTATTAAACTTTTCTCGCGCTTTGCAACAAGCTTTAAGGAT
TACTTCAAAAATCGCGCCAATTGTTTTTCAGCCGACGACATTAGCTCCAGTTCCTGCCAC
CGTATTGTGAATGACAACGCTGAGATTTTTTTTTCCAATGCGCTGGTTTATCGTCGTATT
GTTAAGAGCCTTAGTAACGACGACATTAATAAAATTAGCGGTGATATGAAGGATAGCTTG
AAAGAAATGAGTCTGGAAGAGATCTATAGTTACGAGAAGTACGGCGAATTTATTACCCAG
GAGGGCATTTCATTTTACAATGATATCTGTGGAAAAGTCAACTCCTTTATGAACTTGTAT
TGCCAAAAGAATAAAGAAAACAAAAACCTGTACAAACTGCAAAAGTTACACAAGCAGATT
TTGTGTATCGCAGACACGTCATACGAAGTACCGTACAAGTTTGAGTCCGATGAAGAAGTG
TACCAAAGCGTTAATGGCTTTTTGGATAACATTTCGAGCAAACATATCGTAGAGCGTTTG
CGTAAGATTGGTGATAATTACAACGGTTACAATTTAGACAAAATCTATATCGTCTCTAAG
TTTTACGAAAGTGTTTCTCAGAAAACTTACCGCGATTGGGAGACGATCAACACTGCGCTG
GAGATTCATTACAATAATATCCTTCCAGGTAACGGTAAAAGCAAAGCTGATAAGGTGAAA
AAGGCGGTTAAAAATGACCTTCAAAAGTCTATCACAGAAATCAACGAATTGGTCAGCAAT
TATAAGCTTTGCAGTGACGATAACATTAAGGCCGAGACTTACATCCATGAGATCTCTCAC
ATTCTTAATAATTTTGAAGCGCAAGAGCTGAAATACAATCCTGAAATCCATCTGGTCGAA
AGTGAATTAAAAGCCTCCGAATTAAAAAATGTCTTGGACGTGATCATGAATGCGTTCCAT
TGGTGCTCAGTTTTTATGACGGAAGAGTTGGTGGACAAAGACAACAATTTTTACGCCGAG
CTTGAGGAAATTTACGACGAAATTTACCCCGTTATTTCGTTATACAACCTTGTGCGTAAT
TACGTTACACAAAAGCCCTATTCGACAAAGAAAATCAAGTTAAATTTCGGGATTCCCACA
TTAGCTGATGGATGGTCCAAATCCAAAGAATACTCGAATAACGCTATCATCCTTATGCGT
GATAATTTGTACTACTTAGGCATCTTCAATGCGAAGAACAAACCTGACAAGAAAATTATC
GAAGGAAACACTTCGGAGAACAAAGGTGATTATAAAAAGATGATCTACAACTTGCTTCCC
GGGCCAAACAAAATGATTCCCAAGGTATTTTTGAGTTCTAAAACCGGTGTCGAAACTTAC
AAACCAAGTGCTTATATTTTGGAAGGATACAAACAGAACAAACATATCAAGTCTTCGAAA
GACTTCGATATTACGTTCTGCCACGATCTGATCGATTACTTCAAGAACTGTATTGCTATT
CACCCCGAGTGGAAGAACTTTGGATTTGATTTCTCCGACACGTCCACTTATGAAGATATC
TCTGGCTTCTATCGCGAGGTTGAATTACAAGGGTATAAGATTGACTGGACTTATATTTCG
GAGAAGGATATCGATCTTTTGCAAGAAAAAGGGCAACTTTATTTATTTCAGATCTATAAC
AAGGACTTTTCAAAAAAGAGCACTGGAAATGACAATCTGCATACCATGTACCTTAAGAAC
CTGTTCTCGGAAGAGAACCTGAAGGACATTGTACTTAAACTGAATGGAGAGGCAGAGATC
TTCTTTCGCAAATCAAGCATTAAGAACCCAATTATTCACAAAAAGGGGAGTATCTTAGTA
AATCGCACATATGAGGCTGAGGAAAAAGATCAGTTTGGTAACATTCAGATCGTGCGTAAG
AACATTCCTGAAAATATCTATCAGGAACTTTATAAGTATTTCAACGATAAAAGTGATAAA
GAGCTGAGTGACGAAGCGGCTAAACTTAAGAATGTTGTGGGACACCATGAGGCAGCAACC
AATATTGTGAAGGATTATCGCTATACGTACGACAAATACTTTTTACACATGCCCATCACT
ATTAATTTTAAAGCTAATAAGACTGGCTTCATTAACGATCGCATCCTGCAGTACATTGCT
AAGGAAAAGGATCTTCACGTTATCGGTATCGATCGCGGGGAGCGTAATCTTATCTACGTC
TCTGTCATTGACACGTGTGGCAATATTGTGGAGCAAAAGTCCTTCAATATTGTTAACGGC
TATGACTATCAGATTAAATTGAAACAGCAGGAAGGTGCGCGTCAGATTGCCCGCAAGGAA
TGGAAGGAAATTGGCAAGATCAAAGAAATTAAGGAGGGCTACTTAAGCTTAGTAATTCAC
GAAATTAGTAAAATGGTTATCAAATACAACGCCATCATCGCGATGGAGGATCTTTCGTAC
GGGTTTAAGAAAGGTCGTTTTAAAGTGGAGCGTCAGGTGTACCAGAAATTTGAAACTATG
CTTATTAACAAACTTAACTACCTGGTTTTCAAGGATATCAGTATTACTGAAAACGGGGGG
CTGTTAAAAGGGTATCAATTAACTTACATTCCAGACAAATTAAAGAACGTTGGACATCAG
TGTGGCTGCATTTTTTATGTACCAGCTGCATACACTTCAAAGATCGATCCTACGACTGGG
TTCGTGAACATTTTTAAGTTTAAAGACTTGACGGTAGATGCCAAGCGCGAATTCATCAAG
AAATTCGACAGCATTCGCTACGACTCTGAGAAAAATCTTTTCTGTTTCACATTCGATTAT
AACAATTTCATTACGCAGAACACAGTAATGTCCAAGTCTTCTTGGAGTGTTTATACATAT
GGTGTCCGCATTAAGCGCCGTTTCGTCAACGGCCGCTTCAGTAATGAGAGCGATACTATT
GACATCACAAAAGACATGGAAAAAACACTGGAAATGACCGACATCAATTGGCGTGACGGC
CATGACTTACGTCAGGATATCATTGATTATGAGATCGTTCAACACATCTTCGAAATCTTT
CGCTTGACTGTTCAAATGCGCAATTCCTTGTCGGAATTGGAGGACCGTGATTATGACCGC
TTAATTTCCCCCGTCTTAAATGAAAACAATATTTTTTATGACTCTGCAAAAGCTGGAGAT
GCTCTGCCGAAAGACGCCGATGCAAATGGGGCATATTGCATTGCTTTAAAGGGGCTTTAC
GAGATCAAGCAAATCACCGAAAACTGGAAAGAGGATGGAAAGTTTTCGCGTGATAAACTG
AAGATCTCTAACAAAGACTGGTTCGACTTTATCCAGAACAAGCGTTATTTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 66
AGCCCAGCGGCTAAAAAAAAGAAACTGGATGGCAGCGTGGATATGAACAACGGCACCAAT
AACTTCCAAAACTTCATCGGGATCTCTAGCCTTCAGAAGACGCTTCGCAATGCTCTTATC
CCAACTGAGACCACTCAACAATTTATTGTGAAGAATGGAATTATTAAAGAGGACGAACTG
CGTGGCGAGAATCGTCAGATCTTAAAGGACATTATGGATGATTATTACCGTGGATTCATC
TCCGAAACATTATCGTCGATCGATGATATCGATTGGACTTCTCTGTTCGAGAAAATGGAA
ATTCAATTGAAAAACGGAGATAATAAAGATACGCTTATCAAAGAACAGACGGAATATCGT
AAAGCGATTCATAAGAAATTCGCAAATGACGATCGTTTCAAAAATATGTTCAGTGCCAAG
CTTATTTCGGACATTTTACCTGAATTTGTAATTCATAATAATAACTACTCAGCAAGTGAG
AAGGAGGAGAAAACCCAAGTTATTAAACTGTTCTCTCGTTTCGCAACGTCCTTTAAAGAT
TACTTTAAAAACCGCGCGAATTGCTTTAGCGCTGACGACATTTCCAGCTCATCCTGTCAT
CGCATCGTAAACGACAATGCGGAAATCTTCTTCAGCAACGCCCTGGTTTACCGCCGCATC
GTCAAAAGCTTATCGAATGACGACATCAATAAGATCTCAGGAGATATGAAGGACTCGCTT
AAGGAGATGTCTCTGGAGGAAATTTATAGTTACGAAAAGTATGGAGAGTTCATTACCCAG
GAGGGAATCTCGTTCTACAATGACATTTGCGGGAAGGTGAACTCCTTCATGAACTTATAC
TGCCAGAAAAACAAAGAGAACAAAAATCTGTATAAATTGCAGAAATTACATAAACAGATT
CTTTGTATTGCTGACACTTCCTACGAAGTACCCTATAAATTCGAGTCAGATGAAGAAGTA
TACCAGTCCGTGAACGGATTTCTGGACAATATCTCCTCAAAACACATCGTGGAACGCTTA
CGTAAAATTGGCGATAATTATAATGGTTACAATCTTGACAAAATTTATATCGTATCTAAA
TTTTACGAGAGTGTGAGCCAAAAGACCTACCGCGACTGGGAGACCATCAACACAGCTTTA
GAAATTCACTATAATAATATCTTACCCGGCAATGGTAAGAGCAAGGCTGACAAGGTAAAA
AAGGCCGTCAAGAATGATTTGCAGAAATCTATTACAGAAATTAATGAGTTAGTCTCCAAC
TATAAGCTTTGTTCCGACGATAACATCAAAGCTGAGACATATATTCATGAGATTAGTCAC
ATTCTTAACAACTTCGAGGCCCAGGAACTTAAGTACAATCCTGAAATTCATCTTGTCGAG
TCTGAGCTGAAAGCTAGTGAATTGAAAAATGTTTTAGACGTTATTATGAACGCATTCCAC
TGGTGCTCTGTGTTTATGACAGAAGAACTGGTCGACAAGGACAATAACTTCTATGCCGAA
CTTGAGGAAATCTACGATGAAATTTACCCTGTAATCTCCTTGTATAATCTTGTACGTAAT
TACGTCACTCAAAAACCTTACAGCACGAAAAAAATTAAATTGAACTTCGGGATTCCTACA
CTTGCCGACGGGTGGTCTAAATCCAAGGAATATAGCAACAATGCCATTATTTTAATGCGC
GACAATCTTTACTATTTAGGAATTTTTAACGCTAAGAACAAGCCCGATAAAAAGATTATT
GAAGGAAACACGTCTGAAAATAAGGGCGACTACAAAAAGATGATTTATAACCTTTTGCCC
GGTCCAAACAAAATGATCCCAAAGGTATTCCTGTCATCCAAAACAGGGGTTGAGACATAT
AAGCCCAGCGCATATATTCTGGAAGGATACAAACAGAATAAACATATCAAAAGCAGCAAA
GATTTTGACATTACTTTTTGCCACGATTTAATCGACTACTTCAAAAACTGTATCGCTATC
CACCCTGAATGGAAGAATTTCGGATTTGATTTCTCAGATACAAGTACGTATGAGGATATC
AGCGGTTTCTATCGCGAAGTTGAACTTCAAGGGTATAAAATTGACTGGACCTACATTAGT
GAGAAGGACATCGACCTGTTACAGGAAAAAGGCCAATTGTACTTGTTTCAGATCTACAAT
AAGGATTTCTCAAAAAAATCGACCGGCAATGATAACTTGCACACCATGTACCTGAAGAAC
CTTTTTTCGGAGGAAAACCTTAAAGACATTGTCCTGAAGTTGAATGGAGAAGCGGAGATT
TTCTTTCGTAAGTCTTCCATTAAAAATCCAATTATTCATAAGAAGGGCAGCATCCTTGTG
AACCGTACGTACGAGGCGGAAGAGAAGGACCAATTCGGTAACATTCAAATCGTCCGCAAG
AACATCCCTGAAAATATTTATCAGGAGCTTTACAAGTATTTCAATGATAAGTCCGACAAG
GAATTATCAGATGAGGCTGCGAAGTTGAAAAATGTTGTTGGTCATCACGAGGCGGCGACG
AATATTGTAAAGGATTATCGCTACACTTATGACAAGTACTTTCTGCACATGCCGATCACC
ATTAATTTCAAGGCGAACAAAACAGGATTTATTAATGACCGCATCTTACAATACATTGCC
AAAGAAAAGGACTTACACGTTATTGGCATTGATCGTGGAGAACGCAACTTAATCTACGTA
AGCGTTATTGACACTTGCGGGAATATCGTAGAACAAAAGAGCTTCAACATCGTGAATGGT
TACGATTACCAGATCAAGCTTAAGCAGCAGGAGGGAGCGCGCCAGATCGCGCGCAAGGAA
TGGAAGGAGATTGGTAAGATCAAGGAAATCAAGGAAGGTTATCTGTCCTTGGTAATCCAC
GAAATTTCGAAAATGGTTATCAAATACAATGCTATTATTGCAATGGAGGACTTGTCCTAC
GGCTTTAAAAAAGGACGCTTTAAGGTGGAGCGCCAGGTTTATCAAAAGTTTGAAACAATG
CTGATTAACAAGCTGAACTATTTGGTCTTTAAAGATATCTCCATCACCGAAAATGGTGGG
CTTTTGAAAGGCTATCAACTTACATATATCCCTGATAAGCTTAAGAATGTGGGTCATCAG
TGCGGGTGCATTTTTTATGTTCCTGCAGCCTACACGTCCAAAATCGATCCTACAACTGGA
TTTGTTAATATCTTCAAATTTAAGGATCTTACCGTCGACGCGAAGCGCGAATTTATCAAG
AAATTCGATAGTATTCGTTATGATTCCGAAAAAAACCTTTTCTGTTTCACCTTTGATTAT
AATAACTTTATCACGCAAAATACTGTCATGAGCAAATCGAGTTGGTCTGTGTACACTTAC
GGAGTACGCATCAAGCGTCGTTTTGTTAATGGGCGCTTCAGTAACGAGTCAGACACGATT
GATATCACAAAAGATATGGAGAAAACGCTGGAGATGACAGACATCAATTGGCGCGATGGT
CATGACTTACGTCAAGACATTATCGATTATGAAATTGTCCAGCATATCTTTGAGATCTTT
CGTTTGACTGTTCAGATGCGCAACAGCCTGTCAGAATTGGAGGATCGTGACTATGATCGC
CTTATTTCTCCCGTCTTAAATGAGAACAATATCTTCTACGACTCAGCCAAGGCTGGAGAT
GCACTGCCAAAAGACGCCGACGCAAATGGGGCCTACTGTATTGCATTGAAGGGGTTGTAC
GAGATCAAACAGATTACAGAAAATTGGAAGGAGGACGGTAAGTTCTCTCGTGATAAGCTG
AAGATTTCTAACAAAGACTGGTTCGATTTCATTCAGAACAAACGTTACCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 67
AGCCCAGCGGCTAAAAAAAAGAAACTGGATGGCAGCGTGGATATGAACAACGGTACCAAT
AACTTTCAGAATTTCATTGGAATCAGCAGCTTACAGAAAACCCTGCGCAATGCACTTATC
CCCACTGAGACAACCCAGCAGTTCATTGTAAAGAACGGGATTATTAAAGAAGATGAGCTT
CGCGGGGAGAATCGTCAGATCTTAAAGGATATTATGGACGATTACTACCGTGGCTTCATT
TCGGAGACGCTGTCGTCGATCGACGACATCGACTGGACATCCTTGTTTGAAAAGATGGAA
ATCCAACTGAAGAATGGCGATAACAAGGACACGTTAATCAAAGAGCAGACGGAATACCGT
AAAGCTATCCACAAAAAGTTCGCTAATGACGACCGCTTTAAGAACATGTTCTCAGCAAAA
CTTATTAGCGATATTTTACCTGAATTTGTCATCCACAATAACAATTACTCCGCGAGTGAA
AAAGAGGAGAAAACCCAGGTGATTAAGCTGTTTTCCCGTTTTGCAACCAGTTTCAAGGAC
TATTTTAAGAATCGTGCTAATTGTTTCTCTGCAGACGACATTTCCTCGTCGTCCTGCCAT
CGCATTGTTAATGATAATGCTGAAATCTTTTTTTCAAACGCACTTGTGTATCGTCGCATT
GTCAAAAGCTTAAGTAATGACGATATCAATAAGATCTCAGGAGACATGAAGGACTCCCTG
AAAGAAATGTCATTGGAAGAAATTTACTCTTATGAAAAGTATGGAGAATTTATTACGCAG
GAGGGTATCAGCTTCTATAACGACATTTGTGGTAAAGTGAACAGCTTTATGAATCTTTAT
TGTCAAAAGAATAAAGAGAACAAAAATCTGTACAAGCTGCAGAAATTGCATAAACAAATT
CTGTGCATTGCAGATACTTCGTATGAGGTTCCTTACAAATTCGAGTCGGATGAGGAGGTG
TATCAAAGCGTAAACGGATTTTTGGATAACATTAGTAGTAAGCATATTGTGGAACGCCTT
CGCAAGATTGGTGACAACTATAACGGATACAACTTAGACAAGATCTATATTGTCTCGAAG
TTTTACGAAAGTGTTTCCCAAAAGACTTATCGCGACTGGGAGACAATCAACACTGCGCTG
GAAATTCACTATAACAATATCTTGCCGGGGAACGGAAAAAGTAAGGCAGATAAGGTGAAG
AAAGCAGTCAAAAATGATCTGCAAAAAAGCATTACTGAAATTAACGAACTTGTGTCAAAT
TACAAATTGTGTTCGGATGACAATATTAAAGCGGAAACGTATATCCACGAGATCTCGCAC
ATTCTTAATAATTTCGAGGCGCAGGAATTAAAGTATAATCCTGAGATCCATTTGGTGGAA
TCAGAACTTAAAGCTAGTGAACTGAAAAATGTCCTGGACGTTATTATGAATGCATTTCAC
TGGTGTTCTGTCTTTATGACAGAAGAACTTGTCGACAAAGACAACAACTTTTATGCGGAA
TTAGAAGAGATTTACGACGAAATTTATCCCGTTATTTCGTTATATAATTTAGTTCGTAAT
TACGTGACTCAGAAACCCTACAGCACAAAAAAGATTAAATTAAACTTTGGGATTCCGACT
CTTGCTGATGGATGGAGCAAGTCCAAGGAGTACTCTAATAACGCCATTATCTTGATGCGT
GACAACCTGTACTACCTGGGCATTTTTAACGCTAAAAACAAACCCGACAAAAAGATCATT
GAAGGGAACACCTCGGAAAATAAGGGGGACTATAAAAAAATGATCTACAATCTGTTGCCA
GGCCCAAATAAGATGATCCCAAAGGTTTTTTTATCTTCCAAAACTGGCGTAGAAACTTAC
AAGCCGAGCGCATACATCCTTGAAGGATATAAACAAAACAAACATATCAAAAGTTCAAAG
GACTTCGATATTACGTTCTGCCATGATTTAATCGATTATTTCAAGAATTGCATCGCGATT
CACCCAGAGTGGAAAAACTTTGGGTTTGATTTTTCAGACACCAGCACTTACGAGGATATT
AGTGGATTCTATCGTGAGGTTGAACTGCAGGGCTATAAAATTGACTGGACCTATATTTCT
GAAAAAGATATTGATCTGCTTCAGGAGAAAGGCCAATTGTACTTATTTCAAATCTATAAC
AAGGATTTCTCCAAGAAGTCCACGGGTAATGACAACTTACACACAATGTATCTGAAGAAT
CTGTTTAGTGAGGAGAACTTGAAGGACATTGTGCTGAAGCTTAATGGCGAGGCCGAAATC
TTTTTTCGTAAGTCCTCCATTAAAAACCCTATTATCCATAAGAAAGGGAGTATTCTTGTC
AACCGCACGTATGAGGCCGAAGAAAAGGACCAATTCGGAAACATCCAAATTGTCCGTAAA
AATATTCCTGAGAACATTTACCAGGAGCTTTACAAGTATTTCAACGACAAGAGTGATAAA
GAACTTTCAGATGAGGCGGCGAAACTGAAGAATGTAGTGGGGCACCACGAAGCTGCCACG
AATATTGTAAAGGATTACCGTTACACCTACGACAAGTACTTTTTGCATATGCCCATCACA
ATTAATTTTAAGGCCAATAAAACTGGTTTTATCAACGATCGTATCTTACAGTACATTGCT
AAGGAAAAAGATCTGCACGTTATCGGTATCGATCGCGGGGAACGCAATCTGATTTATGTT
AGTGTGATTGACACGTGCGGAAATATTGTTGAGCAGAAGAGCTTTAATATCGTAAATGGA
TATGACTATCAAATTAAACTGAAGCAACAGGAAGGGGCCCGCCAGATTGCCCGCAAGGAG
TGGAAAGAAATTGGAAAGATCAAGGAGATTAAAGAAGGGTACCTTTCCCTTGTTATCCAC
GAAATCTCGAAAATGGTGATCAAGTACAATGCCATTATTGCTATGGAGGATCTGTCATAT
GGGTTTAAGAAAGGCCGCTTTAAGGTGGAACGTCAGGTTTACCAGAAGTTTGAGACCATG
CTTATCAATAAGCTGAATTATCTTGTCTTCAAAGACATCTCAATCACAGAGAACGGCGGG
CTGTTAAAAGGATATCAGCTGACCTATATCCCCGACAAACTGAAAAATGTCGGGCACCAA
TGCGGCTGTATTTTCTACGTGCCCGCTGCATACACATCTAAAATTGACCCAACGACTGGA
TTCGTAAATATTTTTAAGTTTAAGGATCTTACGGTAGATGCAAAGCGCGAATTTATCAAG
AAATTTGATAGTATCCGTTACGACAGCGAGAAAAACTTATTTTGTTTTACGTTCGATTAT
AACAACTTCATCACGCAAAATACCGTCATGTCAAAATCTTCCTGGTCAGTCTATACGTAT
GGCGTCCGTATCAAGCGCCGCTTCGTCAACGGGCGTTTTTCAAACGAGTCAGATACCATC
GATATCACCAAAGATATGGAAAAAACATTGGAGATGACGGACATCAATTGGCGCGATGGT
CATGACTTACGCCAGGACATTATTGACTACGAAATCGTACAACATATTTTTGAGATTTTC
CGTCTGACCGTGCAAATGCGCAACTCATTATCCGAACTTGAGGATCGTGATTACGACCGC
TTGATCAGTCCTGTTCTGAACGAGAATAATATTTTTTACGACAGTGCCAAGGCGGGAGAC
GCACTGCCCAAGGACGCTGACGCTAACGGAGCTTATTGTATTGCGTTGAAGGGACTTTAC
GAAATCAAGCAAATCACTGAAAACTGGAAGGAGGATGGTAAATTCTCACGCGACAAGTTG
AAAATTTCGAACAAGGACTGGTTCGATTTCATCCAAAACAAGCGTTATTTAAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 68
AGCCCAGCGGCTAAAAAAAAGAAACTGGATGGCAGCGTGGATATGAACAACGGGACTAAT
AACTTCCAGAACTTCATCGGTATTTCATCATTACAAAAAACGCTTCGTAACGCCTTGATC
CCAACAGAAACGACCCAACAATTTATTGTAAAAAACGGCATCATCAAAGAAGACGAACTG
CGTGGCGAAAATCGCCAAATTTTGAAGGACATTATGGATGACTATTATCGTGGGTTTATC
TCGGAGACATTATCCTCCATCGACGACATTGATTGGACGAGTCTTTTTGAGAAAATGGAG
ATCCAGCTTAAAAATGGTGATAACAAGGATACATTGATCAAGGAGCAAACCGAGTACCGC
AAGGCCATCCATAAGAAGTTCGCAAATGACGACCGCTTCAAAAATATGTTTAGTGCCAAA
TTGATCTCGGATATCCTTCCTGAGTTCGTAATTCACAACAATAATTATAGCGCATCCGAA
AAGGAGGAAAAGACTCAAGTCATTAAGCTTTTCAGTCGCTTTGCTACCTCGTTTAAGGAC
TATTTCAAGAACCGCGCGAACTGCTTCTCAGCGGATGACATTTCTTCCTCGTCGTGTCAC
CGCATCGTGAATGATAATGCGGAGATCTTCTTTAGTAATGCCTTGGTATACCGCCGCATT
GTTAAATCCCTGTCTAACGACGATATCAATAAGATCTCAGGAGATATGAAGGATAGCCTT
AAAGAAATGTCTCTGGAAGAAATTTACTCCTATGAAAAGTACGGTGAGTTTATCACCCAA
GAGGGGATTAGCTTTTATAACGATATCTGCGGGAAGGTGAATTCGTTTATGAACCTTTAT
TGTCAAAAGAATAAGGAGAATAAGAACTTATATAAGCTTCAGAAACTGCATAAACAAATC
TTATGCATTGCCGATACTAGCTATGAAGTTCCGTATAAATTCGAGAGCGATGAAGAAGTT
TATCAGAGCGTCAATGGGTTCTTGGATAACATTTCATCAAAACACATCGTGGAACGTCTG
CGTAAGATTGGGGATAACTACAACGGATATAATCTTGACAAAATTTATATTGTATCTAAA
TTCTATGAGTCGGTGAGTCAAAAGACCTACCGTGATTGGGAAACAATCAATACCGCGTTA
GAAATCCACTATAACAACATTCTGCCAGGGAATGGTAAAAGTAAAGCGGACAAAGTCAAG
AAGGCTGTGAAGAACGATCTGCAAAAGAGTATTACAGAGATTAACGAATTAGTCTCCAAT
TATAAGTTATGCTCGGACGATAACATTAAGGCGGAGACGTATATTCATGAGATTTCGCAT
ATTCTTAACAACTTCGAGGCACAAGAGCTTAAGTATAACCCAGAGATTCACCTTGTCGAA
TCGGAGCTGAAGGCATCGGAATTAAAAAATGTCTTAGATGTAATCATGAACGCGTTCCAT
TGGTGCAGTGTTTTCATGACTGAGGAGTTAGTTGACAAGGACAATAACTTCTACGCAGAA
TTAGAAGAGATCTATGATGAGATTTATCCAGTGATTTCGCTGTATAATCTGGTACGTAAT
TACGTCACTCAAAAGCCCTACTCAACAAAAAAAATTAAGCTGAACTTCGGAATTCCGACT
CTGGCCGACGGGTGGTCCAAGTCAAAGGAGTATTCTAATAATGCTATCATCCTGATGCGC
GATAACTTATACTATTTGGGAATTTTCAATGCCAAAAATAAACCAGATAAAAAGATTATC
GAAGGTAATACAAGCGAGAATAAGGGTGACTATAAGAAAATGATTTACAATCTTCTTCCA
GGCCCTAACAAGATGATTCCCAAAGTTTTTTTGTCCAGTAAAACAGGGGTCGAAACTTAC
AAGCCCAGTGCCTATATCCTTGAAGGGTACAAGCAGAATAAGCACATCAAATCCTCGAAA
GACTTTGATATTACATTTTGTCATGACTTAATCGATTATTTTAAGAACTGTATCGCAATC
CATCCAGAATGGAAGAACTTCGGGTTTGATTTCTCTGATACTTCCACGTATGAGGATATT
TCCGGGTTCTACCGCGAAGTAGAGCTTCAGGGCTATAAAATTGACTGGACATATATTTCA
GAAAAAGACATCGATCTGTTACAAGAAAAAGGACAGTTGTATCTGTTTCAAATCTATAAT
AAGGATTTCTCCAAAAAGTCAACTGGAAATGATAACTTACATACAATGTATCTGAAAAAT
CTTTTTAGTGAAGAGAATTTGAAGGATATCGTGCTGAAGTTAAATGGCGAAGCAGAGATC
TTCTTCCGCAAGTCCTCGATCAAGAATCCTATCATCCACAAGAAAGGTAGTATTCTGGTT
AACCGCACGTACGAGGCCGAGGAAAAAGACCAGTTCGGTAATATCCAGATTGTACGTAAG
AATATTCCTGAAAATATTTACCAGGAATTATACAAGTATTTTAACGACAAATCGGATAAG
GAGCTTTCAGATGAGGCCGCAAAGTTGAAGAACGTCGTAGGACACCATGAGGCCGCTACG
AATATCGTCAAGGACTACCGCTATACGTATGACAAGTACTTCCTGCACATGCCTATTACT
ATCAATTTCAAAGCTAATAAAACAGGATTCATCAATGATCGTATCCTTCAGTACATTGCC
AAAGAAAAAGATCTGCACGTAATCGGAATCGACCGTGGCGAACGTAATCTGATTTACGTA
TCAGTTATCGACACATGTGGTAACATCGTGGAGCAGAAATCTTTTAACATTGTTAACGGC
TATGATTATCAGATTAAGCTTAAACAGCAGGAGGGGGCACGCCAAATCGCTCGTAAAGAA
TGGAAGGAGATTGGAAAGATTAAAGAGATTAAAGAGGGGTACCTTTCGCTGGTTATTCAC
GAAATTTCCAAGATGGTGATTAAGTACAATGCAATCATCGCGATGGAAGATCTTAGTTAC
GGATTCAAAAAGGGACGCTTCAAAGTTGAGCGTCAGGTCTACCAGAAATTTGAAACGATG
CTGATTAACAAATTGAATTACTTGGTATTCAAAGATATCTCAATTACTGAAAATGGTGGC
TTATTAAAGGGTTACCAGCTTACCTATATCCCGGATAAGCTGAAGAACGTGGGCCATCAA
TGCGGCTGCATCTTTTACGTCCCTGCCGCATATACCTCTAAAATTGACCCCACCACCGGA
TTCGTAAATATTTTTAAATTCAAGGACCTGACGGTGGACGCCAAGCGCGAATTCATCAAA
AAATTCGACTCAATCCGCTATGATTCCGAAAAAAATCTTTTCTGCTTTACGTTCGATTAT
AATAACTTCATTACCCAAAACACGGTGATGTCAAAATCGTCCTGGAGCGTGTATACTTAT
GGAGTGCGTATCAAGCGCCGCTTTGTTAATGGGCGCTTCAGTAACGAAAGCGATACCATC
GACATTACCAAAGACATGGAGAAGACGCTTGAAATGACGGATATCAATTGGCGTGACGGA
CACGATCTTCGTCAGGATATCATCGACTACGAGATTGTGCAACATATCTTTGAGATTTTC
CGTTTAACTGTTCAAATGCGTAACTCCTTGTCCGAATTGGAAGACCGTGATTACGACCGC
TTGATTTCACCAGTGCTTAACGAGAATAACATCTTCTACGACTCCGCCAAAGCAGGCGAT
GCCCTGCCAAAGGACGCTGATGCAAATGGTGCATACTGTATCGCGTTGAAGGGCTTATAC
GAGATTAAGCAAATCACCGAAAATTGGAAAGAGGATGGAAAGTTCAGTCGCGATAAGCTG
AAGATCTCTAATAAAGATTGGTTTGACTTTATCCAGAACAAACGTTATTTAAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 69
AGCCCAGCGGCTAAAAAAAAGAAACTGGATGGCAGCGTGGATATGAACAACGGTACCAAT
AATTTCCAAAATTTCATCGGAATCTCATCCTTGCAAAAAACCTTGCGCAATGCTTTGATC
CCCACCGAAACCACGCAGCAGTTCATCGTGAAAAACGGCATTATCAAAGAGGATGAGTTG
CGCGGGGAAAACCGTCAAATTCTTAAGGATATCATGGACGATTACTACCGTGGGTTTATC
AGTGAGACCCTGTCAAGCATTGACGACATTGACTGGACCAGCTTATTTGAGAAGATGGAG
ATTCAATTAAAGAACGGGGACAATAAGGACACGCTTATCAAAGAGCAGACAGAATACCGT
AAAGCGATTCATAAGAAATTTGCAAATGACGATCGCTTCAAGAACATGTTTTCAGCAAAA
TTAATCAGCGACATCCTTCCCGAATTTGTGATTCATAATAACAACTATTCGGCTAGCGAA
AAAGAGGAGAAAACTCAGGTTATTAAGCTTTTCTCGCGTTTTGCCACTTCGTTCAAAGAC
TATTTTAAGAATCGCGCAAACTGCTTTTCGGCTGATGATATTTCCAGTTCTAGCTGCCAT
CGTATCGTTAACGATAATGCTGAGATTTTCTTCTCTAATGCCCTGGTGTATCGTCGTATC
GTTAAATCTTTGAGCAACGACGATATTAATAAGATTTCAGGCGACATGAAGGATTCTTTA
AAGGAGATGTCTTTAGAAGAGATTTATTCCTATGAGAAATATGGCGAGTTTATCACCCAA
GAAGGAATTTCGTTCTACAACGACATCTGTGGCAAAGTGAACAGCTTCATGAATTTATAC
TGCCAAAAGAATAAGGAGAATAAAAATTTATATAAACTGCAGAAACTGCATAAGCAAATT
CTTTGCATTGCAGACACCTCTTATGAAGTTCCTTATAAGTTTGAATCGGACGAGGAGGTA
TATCAGAGTGTGAACGGGTTCCTGGACAATATTTCATCCAAGCATATTGTTGAACGTTTA
CGCAAAATTGGAGACAATTACAATGGGTATAACCTTGACAAAATTTACATCGTGTCGAAG
TTTTACGAATCGGTAAGCCAGAAGACCTATCGTGACTGGGAAACTATCAATACCGCCTTA
GAAATTCATTACAACAATATTCTTCCTGGTAACGGCAAAAGCAAAGCCGATAAGGTAAAG
AAGGCTGTCAAGAACGACCTGCAAAAGTCTATCACAGAGATCAACGAGTTAGTCTCTAAC
TACAAATTATGTTCCGACGACAATATTAAAGCCGAAACCTACATCCATGAGATCTCACAC
ATTCTTAACAATTTTGAGGCCCAGGAGCTGAAATATAACCCAGAAATTCACCTTGTAGAG
AGCGAATTAAAAGCCTCCGAGCTGAAGAACGTTTTGGATGTAATCATGAACGCATTTCAT
TGGTGCAGCGTATTTATGACAGAGGAGTTGGTCGACAAGGACAATAACTTTTACGCCGAG
CTTGAAGAAATCTACGATGAAATTTACCCGGTAATTAGTTTATATAATTTAGTTCGCAAC
TACGTAACTCAGAAACCCTACAGTACCAAGAAGATTAAATTGAACTTTGGGATCCCGACA
CTTGCTGACGGTTGGAGTAAATCAAAAGAATACTCCAATAATGCAATTATCCTGATGCGC
GACAATCTTTACTACTTGGGGATCTTTAACGCAAAGAACAAACCAGATAAGAAAATCATC
GAGGGCAACACCAGCGAGAATAAAGGCGATTACAAGAAAATGATCTATAATCTTTTGCCG
GGACCGAACAAAATGATCCCAAAGGTTTTCCTGTCGTCGAAAACGGGAGTCGAGACATAT
AAACCATCTGCGTACATCTTGGAAGGTTACAAACAGAATAAGCATATTAAGTCTAGTAAA
GACTTCGACATCACCTTTTGTCATGACCTGATTGATTATTTCAAGAACTGTATTGCTATC
CATCCAGAATGGAAAAACTTCGGATTTGACTTCTCCGATACTAGCACCTACGAAGACATT
TCGGGTTTTTATCGCGAAGTAGAGCTTCAAGGGTACAAAATTGATTGGACATATATTAGC
GAGAAAGACATTGATTTGCTTCAAGAGAAGGGACAGTTATATTTATTCCAGATCTACAAC
AAAGACTTCTCGAAGAAATCCACCGGTAATGATAATCTTCACACTATGTACCTGAAGAAT
TTATTTTCAGAGGAAAATCTGAAGGACATTGTACTTAAACTTAATGGAGAAGCCGAAATC
TTCTTCCGCAAGAGTTCCATTAAAAATCCGATTATTCATAAAAAGGGAAGTATCCTTGTG
AACCGCACGTATGAGGCCGAAGAGAAGGATCAGTTTGGGAATATTCAAATTGTCCGCAAA
AACATCCCCGAGAACATCTACCAGGAACTGTATAAATACTTTAATGATAAATCTGATAAA
GAGTTATCAGACGAGGCTGCCAAACTGAAAAACGTAGTCGGTCATCATGAGGCAGCGACC
AATATTGTAAAGGACTACCGTTACACCTACGACAAGTATTTCCTTCACATGCCGATCACG
ATTAATTTTAAGGCTAACAAGACCGGCTTTATCAATGACCGCATCTTGCAGTACATCGCG
AAAGAGAAAGATTTACACGTCATCGGAATTGATCGTGGAGAGCGTAATCTTATCTACGTC
AGCGTCATCGACACCTGTGGAAACATTGTGGAACAAAAAAGTTTTAATATCGTAAACGGC
TACGACTATCAAATTAAACTTAAACAGCAAGAGGGAGCTCGCCAGATCGCTCGCAAAGAG
TGGAAAGAGATTGGGAAAATTAAAGAAATTAAAGAGGGTTACCTGTCGCTGGTAATTCAC
GAAATCTCGAAAATGGTCATCAAATATAATGCAATTATCGCTATGGAGGATCTGTCCTAC
GGGTTCAAGAAGGGACGTTTTAAAGTAGAGCGCCAGGTGTATCAAAAATTCGAAACCATG
TTGATCAATAAGCTTAACTATTTGGTCTTCAAAGATATTTCGATTACGGAGAACGGAGGT
TTGTTGAAAGGATATCAGCTGACGTATATCCCAGACAAGTTGAAAAACGTGGGGCATCAA
TGTGGATGTATTTTCTATGTGCCCGCGGCCTACACGAGTAAGATCGATCCTACCACTGGT
TTCGTCAACATTTTCAAATTTAAAGATCTTACCGTGGATGCGAAGCGCGAATTTATTAAG
AAATTTGATAGCATTCGCTATGATTCCGAAAAGAACCTGTTCTGTTTTACGTTCGACTAT
AACAATTTCATTACCCAAAACACGGTGATGAGCAAATCCTCTTGGTCAGTTTATACATAC
GGTGTACGTATCAAACGCCGTTTCGTTAACGGACGCTTTTCCAATGAGTCTGATACAATC
GATATCACGAAAGATATGGAAAAAACATTAGAGATGACTGATATCAACTGGCGTGACGGG
CACGACCTGCGTCAAGACATTATTGACTACGAGATTGTGCAGCATATCTTCGAAATCTTT
CGCTTAACTGTGCAAATGCGTAACTCGTTATCCGAGTTAGAAGACCGTGACTACGATCGC
CTGATTTCACCCGTCTTGAACGAAAATAACATCTTCTACGATTCCGCGAAGGCTGGGGAC
GCATTGCCCAAGGACGCAGACGCGAATGGAGCGTACTGTATTGCGCTTAAAGGATTATAT
GAAATCAAGCAGATCACCGAAAATTGGAAGGAGGACGGGAAGTTCTCACGCGACAAACTG
AAGATTTCAAATAAGGACTGGTTCGATTTCATTCAGAATAAGCGTTACCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 70
AGCCCAGCGGCTAAAAAAAAGAAACTGGATGGCAGCGTGGATATGAATAATGGTACGAAC
AACTTTCAGAACTTCATCGGCATCTCCAGCCTTCAAAAGACTTTACGCAACGCATTGATT
CCCACGGAGACTACGCAACAGTTTATCGTAAAAAATGGTATTATCAAAGAAGATGAATTA
CGCGGGGAGAATCGCCAGATTCTTAAGGACATTATGGACGATTATTACCGTGGATTCATC
AGTGAGACACTGAGCTCCATTGATGACATCGACTGGACGTCATTGTTTGAAAAGATGGAA
ATCCAGTTGAAAAATGGCGATAACAAAGATACATTGATTAAAGAGCAGACAGAGTACCGC
AAAGCAATTCACAAGAAATTCGCCAATGATGATCGTTTTAAGAACATGTTTAGTGCCAAG
CTTATTTCGGATATCTTACCCGAATTCGTGATTCACAACAACAATTATTCGGCAAGTGAG
AAAGAGGAAAAGACCCAGGTTATCAAATTGTTTTCGCGCTTCGCCACTTCGTTCAAAGAT
TATTTCAAGAACCGTGCAAACTGTTTCTCCGCTGACGACATCAGTTCCAGCTCATGCCAC
CGTATTGTAAATGACAATGCGGAGATCTTTTTCAGTAATGCCTTAGTATATCGTCGCATT
GTAAAGAGCTTATCTAATGATGACATTAACAAGATCTCGGGTGATATGAAGGACTCACTT
AAGGAGATGAGTCTGGAAGAGATCTACTCCTACGAAAAATACGGGGAATTCATCACCCAG
GAGGGAATTTCATTCTACAACGATATCTGCGGCAAAGTTAACTCCTTTATGAATCTGTAC
TGTCAAAAGAACAAGGAGAATAAAAACCTGTATAAATTGCAGAAACTTCATAAACAAATT
TTGTGTATCGCAGACACGAGTTATGAAGTACCTTATAAATTCGAATCCGACGAAGAGGTA
TATCAGTCCGTAAATGGGTTCCTGGACAATATCAGTAGTAAGCACATTGTGGAACGCTTA
CGCAAAATTGGAGACAATTACAACGGGTATAACCTGGACAAAATCTACATCGTATCCAAA
TTTTATGAAAGCGTGTCTCAAAAAACTTATCGTGATTGGGAAACAATCAACACGGCTCTT
GAGATCCATTACAATAACATCTTGCCGGGTAACGGCAAATCGAAGGCAGACAAAGTTAAA
AAAGCAGTTAAGAACGACTTACAGAAAAGCATTACGGAGATTAACGAGTTAGTAAGTAAT
TACAAATTATGCTCCGACGATAATATCAAAGCTGAAACCTACATCCATGAAATTAGCCAC
ATTTTGAACAATTTCGAAGCGCAGGAGCTGAAATATAACCCTGAAATCCATCTGGTAGAG
TCTGAGTTGAAGGCGTCAGAACTGAAAAACGTTCTTGACGTCATCATGAATGCCTTTCAC
TGGTGTAGTGTTTTTATGACTGAGGAGCTTGTAGATAAGGACAACAACTTCTATGCTGAA
CTTGAAGAGATCTACGATGAAATCTACCCCGTAATCAGTCTGTATAATTTAGTTCGTAAC
TACGTCACGCAGAAACCCTATTCGACTAAGAAAATTAAGCTGAACTTTGGGATCCCTACT
TTGGCAGACGGGTGGAGCAAGAGTAAAGAATACAGTAATAATGCAATTATCTTGATGCGC
GATAACTTATATTACTTAGGTATTTTCAATGCTAAGAACAAACCTGATAAGAAGATTATC
GAAGGAAATACGAGTGAGAATAAGGGAGACTACAAAAAGATGATTTACAACTTGCTGCCA
GGGCCTAATAAGATGATTCCAAAAGTTTTTCTGTCGAGCAAGACAGGGGTTGAAACTTAT
AAGCCATCCGCTTATATCCTTGAGGGGTACAAGCAGAATAAGCATATCAAGTCCTCCAAA
GATTTTGATATTACATTTTGCCACGACTTAATTGATTACTTCAAGAACTGCATCGCAATC
CATCCCGAATGGAAGAATTTCGGCTTCGATTTCTCAGATACGTCCACGTATGAGGATATC
TCAGGCTTTTACCGCGAAGTTGAGCTGCAAGGTTATAAAATTGATTGGACATACATCTCC
GAAAAAGACATTGATCTTTTACAGGAAAAGGGCCAATTATACTTATTTCAAATCTATAAC
AAAGATTTTAGCAAGAAGTCCACAGGTAATGATAACCTGCATACGATGTATTTGAAAAAT
CTTTTCAGTGAAGAGAATTTGAAGGATATCGTCCTGAAGCTGAACGGTGAGGCTGAGATC
TTCTTCCGCAAATCGTCTATCAAAAACCCCATCATTCACAAAAAGGGAAGTATCTTAGTA
AACCGCACTTATGAAGCGGAGGAAAAGGATCAGTTCGGGAACATCCAGATCGTGCGCAAG
AACATTCCAGAAAACATCTATCAGGAACTTTACAAATATTTCAATGACAAGTCTGATAAA
GAATTATCAGACGAGGCGGCGAAACTTAAAAATGTTGTTGGACACCACGAAGCAGCGACG
AATATTGTAAAGGATTATCGCTACACATACGATAAATACTTTTTGCACATGCCAATCACC
ATTAACTTTAAGGCGAACAAGACAGGTTTCATTAACGACCGTATTCTGCAATATATCGCA
AAGGAAAAAGACCTGCACGTTATTGGGATCGATCGTGGCGAACGCAATTTGATCTACGTA
AGCGTTATCGACACTTGCGGAAATATCGTTGAACAAAAAAGCTTTAATATCGTCAATGGA
TACGATTACCAAATCAAGCTGAAACAACAAGAAGGGGCACGTCAGATCGCTCGTAAAGAA
TGGAAAGAGATTGGTAAGATCAAAGAGATTAAAGAAGGGTATCTTTCTTTAGTAATTCAC
GAGATTTCGAAAATGGTTATTAAATACAATGCGATTATTGCTATGGAAGACTTAAGCTAC
GGCTTTAAGAAAGGTCGCTTCAAAGTGGAGCGCCAAGTGTATCAGAAGTTTGAAACGATG
TTGATTAACAAATTAAATTACCTGGTCTTTAAGGACATCAGTATCACAGAAAATGGGGGG
TTGCTTAAAGGGTACCAGCTTACATACATCCCTGATAAACTGAAAAATGTCGGTCATCAG
TGCGGATGTATCTTCTATGTACCAGCAGCCTATACCAGTAAGATTGACCCTACTACTGGC
TTTGTGAATATTTTTAAATTCAAGGATTTAACCGTGGACGCCAAGCGTGAATTTATTAAA
AAATTTGATTCGATTCGCTACGACAGTGAGAAAAACCTTTTCTGCTTTACCTTTGACTAC
AACAATTTTATTACCCAGAACACCGTAATGTCAAAGAGTTCGTGGTCTGTATATACCTAC
GGTGTTCGCATCAAGCGCCGCTTCGTAAACGGGCGTTTCAGTAACGAATCTGACACCATC
GACATCACTAAAGATATGGAGAAGACATTGGAAATGACGGACATTAATTGGCGTGATGGC
CATGACTTACGTCAGGACATTATTGATTACGAAATTGTGCAGCATATCTTCGAGATTTTC
CGTTTGACAGTTCAGATGCGCAACTCACTGAGTGAGTTAGAAGATCGCGATTACGACCGT
CTGATCTCACCGGTCCTTAATGAAAACAACATTTTCTACGACTCAGCAAAGGCGGGTGAT
GCCCTGCCAAAGGATGCGGACGCTAATGGCGCCTACTGCATCGCCCTGAAAGGATTGTAT
GAAATTAAGCAGATTACAGAAAATTGGAAGGAAGATGGTAAATTTAGCCGTGATAAATTA
AAAATCTCGAACAAGGATTGGTTCGATTTTATTCAGAACAAACGTTATTTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 71
AGCCCAGCGGCTAAAAAAAAGAAACTGGATGGCAGCGTGGATATGAACAATGGAACAAAT
AATTTTCAAAATTTTATCGGCATCTCAAGTCTTCAAAAAACCCTTCGCAATGCCCTGATT
CCAACTGAAACAACCCAGCAATTTATCGTCAAGAACGGCATCATTAAGGAAGACGAGTTA
CGCGGGGAGAACCGTCAAATCCTGAAAGATATCATGGATGACTACTATCGTGGGTTCATT
TCGGAAACCTTGTCTTCAATCGACGACATTGACTGGACGAGTCTTTTCGAGAAAATGGAA
ATTCAGCTTAAAAATGGAGACAACAAGGATACTCTGATTAAGGAACAGACAGAATATCGC
AAAGCTATCCACAAAAAGTTCGCTAATGATGATCGTTTCAAAAATATGTTTTCTGCTAAA
TTGATTTCCGATATCTTGCCTGAATTTGTAATCCACAACAACAATTATTCTGCTTCCGAG
AAGGAAGAGAAGACCCAGGTCATTAAATTATTCAGCCGCTTTGCAACCAGCTTTAAAGAC
TACTTTAAGAATCGCGCTAACTGCTTTTCGGCGGATGACATCTCATCATCATCATGCCAC
CGCATTGTGAACGACAATGCGGAGATCTTCTTTTCGAATGCGTTAGTTTATCGTCGCATT
GTCAAAAGTCTTAGCAATGATGACATCAACAAGATCTCAGGAGACATGAAAGATTCCTTA
AAGGAGATGTCTCTTGAGGAAATCTATTCGTATGAGAAATACGGCGAGTTCATTACCCAG
GAAGGTATTAGTTTCTACAATGATATCTGCGGCAAAGTAAATTCTTTTATGAATCTGTAT
TGCCAAAAAAACAAAGAAAACAAGAATCTTTATAAGTTACAAAAGTTACATAAGCAAATT
CTGTGCATCGCTGATACATCTTATGAGGTACCCTACAAATTTGAAAGTGATGAGGAGGTC
TATCAGAGTGTCAACGGCTTCTTAGACAACATCTCTTCCAAACATATCGTGGAACGCCTG
CGTAAAATCGGAGATAACTACAACGGATATAACTTAGATAAAATCTACATCGTGTCCAAG
TTTTATGAAAGTGTGAGCCAAAAAACATATCGTGACTGGGAAACCATTAACACCGCATTG
GAAATTCACTATAACAACATTTTGCCAGGCAACGGGAAAAGTAAGGCGGACAAAGTTAAG
AAAGCAGTTAAAAATGACCTGCAAAAAAGCATCACTGAAATTAACGAATTGGTATCGAAT
TACAAATTATGTAGCGACGATAATATCAAAGCAGAAACTTACATTCACGAGATTAGTCAC
ATTTTAAATAACTTCGAGGCCCAGGAATTGAAATACAATCCCGAAATTCATTTGGTTGAA
TCAGAACTGAAAGCATCAGAGTTGAAAAATGTGTTAGATGTCATTATGAATGCGTTTCAT
TGGTGCTCTGTGTTCATGACCGAGGAACTGGTTGATAAAGATAACAACTTTTACGCTGAA
TTGGAGGAGATTTACGATGAGATTTACCCGGTCATTTCGCTTTATAACTTAGTGCGCAAT
TATGTGACGCAGAAACCATATTCCACGAAGAAAATCAAACTTAATTTTGGCATCCCTACT
CTGGCTGATGGTTGGTCGAAATCGAAAGAGTACAGCAACAACGCGATCATTCTTATGCGT
GACAATCTTTACTATTTGGGCATTTTTAATGCCAAGAATAAGCCAGATAAGAAAATCATT
GAGGGGAATACTTCCGAGAATAAGGGGGATTACAAAAAGATGATCTATAACTTGCTGCCC
GGCCCCAACAAAATGATTCCTAAGGTTTTCTTGTCAAGCAAGACGGGCGTCGAAACATAT
AAGCCGTCAGCTTATATTCTGGAAGGCTATAAACAGAATAAGCACATCAAGTCTTCCAAG
GACTTTGACATCACTTTTTGCCACGATTTGATCGACTACTTTAAGAACTGTATTGCGATT
CATCCGGAATGGAAGAACTTCGGTTTCGACTTTTCCGATACCTCAACATACGAGGATATC
AGCGGCTTCTACCGTGAAGTCGAGCTTCAAGGCTACAAGATCGATTGGACATATATTTCA
GAGAAGGACATTGATTTGTTACAAGAGAAAGGTCAACTTTACTTATTTCAGATCTATAAC
AAAGACTTTTCGAAGAAATCGACAGGAAACGATAACTTACACACTATGTATTTAAAAAAT
CTGTTTTCGGAGGAAAACCTGAAAGATATTGTGCTGAAACTTAACGGCGAGGCAGAGATC
TTTTTCCGTAAAAGCTCAATCAAGAATCCTATCATCCATAAAAAAGGTAGTATTCTTGTC
AACCGCACATATGAAGCGGAGGAGAAGGACCAATTCGGAAACATCCAAATTGTCCGTAAG
AATATTCCGGAGAACATTTACCAAGAGTTGTATAAATACTTTAACGATAAGTCAGATAAG
GAACTTAGCGATGAGGCGGCGAAGCTTAAAAACGTAGTTGGGCATCATGAAGCTGCTACC
AACATTGTAAAAGATTACCGTTACACCTATGACAAGTATTTCTTGCACATGCCCATTACG
ATCAATTTCAAAGCAAATAAGACAGGCTTTATCAATGATCGCATCCTGCAGTACATTGCT
AAAGAGAAGGATTTGCATGTTATCGGTATTGATCGCGGAGAGCGCAATTTGATCTACGTC
TCCGTAATCGACACTTGCGGTAACATTGTTGAGCAGAAGTCGTTCAACATCGTTAATGGT
TATGATTACCAAATCAAGCTGAAGCAGCAAGAGGGTGCCCGCCAGATCGCGCGTAAGGAA
TGGAAAGAAATCGGGAAAATTAAAGAGATCAAAGAAGGCTATTTGTCTCTGGTAATTCAC
GAAATCAGCAAGATGGTGATCAAGTATAACGCGATCATTGCGATGGAGGATCTTTCTTAT
GGCTTCAAGAAAGGGCGCTTTAAAGTCGAACGCCAGGTCTACCAGAAATTTGAGACAATG
CTTATCAACAAGCTTAACTATCTTGTATTTAAGGATATTTCCATCACTGAGAACGGAGGA
CTTTTAAAGGGGTACCAACTGACGTACATTCCTGATAAGCTGAAGAACGTTGGTCATCAA
TGCGGATGCATCTTCTATGTGCCAGCGGCTTACACCTCCAAAATCGATCCCACTACAGGC
TTTGTCAATATCTTCAAATTCAAGGATTTGACCGTTGACGCGAAGCGCGAGTTTATCAAG
AAGTTTGATAGCATTCGCTACGACAGCGAAAAAAATTTATTTTGTTTTACTTTCGACTAC
AATAACTTTATTACTCAGAACACTGTCATGTCAAAGAGTTCGTGGAGTGTCTACACGTAC
GGAGTACGTATTAAGCGCCGTTTCGTCAACGGACGCTTCTCAAACGAAAGCGACACGATC
GACATCACCAAAGACATGGAAAAAACTCTTGAGATGACGGATATCAATTGGCGCGACGGC
CATGACCTGCGTCAGGATATCATTGATTACGAGATCGTTCAGCACATCTTCGAAATCTTC
CGCCTTACCGTCCAGATGCGCAACAGTTTAAGCGAGCTTGAAGACCGCGACTACGATCGT
TTGATTAGCCCCGTTCTGAACGAGAATAATATTTTCTACGACAGCGCAAAGGCCGGTGAT
GCTTTGCCAAAGGACGCAGACGCGAATGGAGCCTACTGCATCGCCCTGAAGGGCTTATAT
GAGATTAAGCAAATTACCGAAAATTGGAAGGAAGATGGTAAGTTCTCCCGTGATAAGCTT
AAAATTAGCAATAAGGATTGGTTCGACTTCATCCAGAACAAACGTTACCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 72
AGCCCAGCGGCTAAAAAAAAGAAACTGGATGGCAGCGTGGATATGAACAACGGAACAAAC
AATTTCCAAAACTTCATCGGTATCTCTTCGTTGCAGAAGACTCTGCGTAATGCTTTGATC
CCGACGGAGACAACCCAACAATTTATCGTCAAAAACGGTATTATTAAGGAGGACGAGTTA
CGTGGAGAAAATCGTCAAATCCTTAAGGACATCATGGACGATTATTATCGCGGGTTTATT
TCTGAAACCCTGAGCAGTATCGATGATATCGACTGGACCTCACTTTTTGAGAAAATGGAG
ATCCAGTTGAAGAACGGTGATAACAAAGACACTCTGATCAAAGAGCAAACTGAATACCGC
AAGGCAATTCACAAAAAGTTCGCCAACGACGACCGTTTCAAGAATATGTTCTCAGCTAAG
TTAATCAGCGACATTTTGCCAGAGTTCGTTATCCACAACAATAATTATAGTGCTTCAGAG
AAGGAGGAAAAAACCCAAGTGATTAAACTTTTTTCGCGCTTTGCAACCTCATTCAAGGAC
TACTTCAAGAATCGCGCGAATTGCTTCAGTGCGGACGACATTTCTTCTTCAAGTTGCCAT
CGTATCGTTAACGATAACGCGGAAATTTTCTTCTCTAATGCTTTGGTGTATCGCCGCATT
GTAAAATCGCTTAGTAACGATGACATTAATAAGATCTCAGGTGATATGAAAGATTCATTG
AAGGAAATGAGCTTGGAAGAGATTTACAGTTACGAAAAATATGGAGAATTTATTACTCAG
GAAGGCATCTCATTCTATAACGATATCTGCGGGAAGGTAAATTCGTTTATGAACTTATAT
TGCCAGAAAAATAAAGAGAATAAAAATTTGTATAAGCTTCAGAAGTTGCACAAACAGATC
CTGTGCATTGCAGACACCTCGTATGAGGTTCCGTATAAATTTGAGTCCGATGAAGAAGTG
TATCAGTCTGTGAATGGTTTCTTAGATAATATCTCTTCCAAGCATATTGTCGAACGCCTG
CGCAAAATTGGTGATAACTATAACGGATACAATCTGGATAAAATTTACATCGTTTCTAAA
TTTTACGAGTCAGTCTCGCAGAAGACCTACCGCGACTGGGAAACAATTAACACGGCATTG
GAGATTCACTACAATAATATCTTGCCTGGTAACGGTAAGTCTAAGGCAGATAAGGTAAAA
AAAGCTGTGAAAAACGACCTTCAGAAAAGCATCACGGAGATTAATGAGCTGGTGAGTAAT
TACAAATTATGTTCAGACGATAATATTAAAGCTGAAACGTATATCCATGAAATCTCGCAT
ATCTTGAACAACTTCGAGGCCCAAGAACTTAAATATAACCCCGAAATCCATTTAGTCGAG
TCTGAATTGAAAGCGTCGGAATTAAAAAACGTCTTAGACGTCATTATGAACGCGTTTCAC
TGGTGTTCAGTTTTCATGACCGAAGAGCTGGTCGACAAAGACAACAACTTCTATGCGGAA
TTGGAGGAAATCTATGATGAAATCTACCCTGTTATTTCACTGTATAACCTTGTGCGCAAC
TATGTCACTCAGAAGCCGTATTCGACCAAAAAAATTAAATTGAATTTCGGTATCCCTACT
CTTGCAGACGGATGGAGTAAAAGCAAGGAATACAGTAATAACGCCATTATTCTTATGCGC
GACAATTTATACTACCTGGGCATCTTTAACGCAAAGAATAAGCCGGATAAGAAGATTATT
GAGGGTAACACCAGTGAGAACAAGGGCGACTATAAGAAGATGATCTATAACTTATTGCCA
GGTCCAAATAAAATGATCCCAAAAGTATTCTTATCATCAAAGACGGGAGTTGAAACCTAT
AAGCCTAGTGCCTATATTCTTGAGGGATATAAACAGAACAAGCACATTAAGTCGTCTAAG
GATTTTGACATTACGTTCTGCCATGACTTAATCGACTATTTTAAAAACTGTATTGCGATT
CACCCCGAATGGAAGAATTTTGGATTCGATTTTTCGGATACCTCGACCTATGAAGATATT
TCGGGATTTTATCGTGAAGTGGAGTTGCAAGGCTATAAAATCGATTGGACCTATATCTCA
GAAAAAGACATTGATTTATTACAGGAAAAGGGACAACTGTACCTTTTCCAAATTTATAAC
AAGGACTTTTCTAAAAAGTCCACAGGAAATGATAACCTTCACACCATGTACCTGAAGAAC
CTTTTCTCAGAGGAAAACCTGAAGGACATTGTCCTTAAGTTAAATGGAGAAGCGGAGATC
TTTTTCCGTAAATCTAGTATCAAGAATCCGATTATCCATAAAAAAGGTTCGATTTTGGTA
AATCGCACCTATGAAGCGGAAGAGAAAGATCAATTTGGTAACATCCAGATCGTGCGCAAG
AATATCCCGGAGAACATTTACCAAGAGCTGTATAAGTACTTCAATGATAAGTCTGATAAG
GAACTGTCAGATGAAGCTGCGAAATTGAAGAACGTGGTTGGGCATCATGAAGCCGCTACC
AATATCGTCAAGGATTACCGTTATACCTATGACAAATATTTCTTACACATGCCGATTACG
ATCAATTTTAAGGCAAACAAGACAGGATTCATCAACGACCGTATCTTGCAGTATATTGCC
AAAGAGAAGGATCTGCATGTGATCGGTATTGACCGCGGGGAGCGCAATTTAATCTATGTA
TCGGTGATCGATACTTGTGGTAACATCGTAGAACAAAAGAGCTTTAACATCGTGAATGGT
TACGACTATCAGATCAAGCTGAAACAACAGGAAGGAGCCCGCCAGATCGCTCGCAAGGAA
TGGAAAGAAATCGGGAAAATTAAGGAAATCAAGGAAGGCTACCTTTCATTGGTCATTCAC
GAAATTTCGAAAATGGTAATTAAGTACAACGCGATCATCGCCATGGAGGACCTTTCGTAC
GGATTTAAGAAGGGTCGTTTCAAAGTTGAGCGCCAGGTATACCAAAAATTCGAGACTATG
CTTATCAACAAACTTAACTACTTGGTCTTTAAGGACATTTCTATTACCGAAAACGGCGGC
TTACTTAAAGGCTATCAATTGACATATATTCCCGACAAACTGAAGAATGTTGGACATCAA
TGCGGGTGTATTTTCTATGTGCCGGCAGCTTACACTAGTAAGATCGACCCTACAACCGGG
TTCGTAAACATTTTTAAATTCAAAGACTTAACAGTCGATGCGAAGCGTGAATTTATTAAG
AAGTTTGATAGTATCCGCTATGACAGTGAAAAGAACTTGTTTTGCTTTACGTTCGACTAC
AATAACTTTATTACACAGAACACGGTCATGTCTAAATCATCATGGTCGGTTTACACATAT
GGGGTGCGCATCAAGCGTCGCTTTGTAAATGGCCGTTTTAGTAATGAGAGCGACACAATC
GACATCACAAAGGATATGGAGAAAACTCTTGAGATGACAGACATCAATTGGCGTGACGGT
CATGACTTACGCCAAGATATCATCGACTACGAAATCGTACAGCATATTTTTGAGATTTTT
CGTCTTACTGTGCAAATGCGTAATTCTTTATCCGAACTGGAAGATCGTGATTACGACCGC
TTGATTAGTCCCGTCTTAAATGAGAACAATATTTTCTATGATTCTGCGAAAGCCGGAGAT
GCACTGCCCAAAGACGCTGATGCCAATGGCGCGTATTGCATTGCATTAAAAGGATTATAT
GAGATTAAACAGATTACCGAAAATTGGAAAGAGGACGGTAAATTCTCACGCGATAAATTG
AAGATTTCTAACAAGGACTGGTTCGACTTTATCCAAAATAAACGTTATCTTAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 73
AGCCCAGCGGCTAAAAAAAAGAAACTGGATGGCAGCGTGGATATGAATAACGGTACCAAC
AACTTTCAGAATTTCATTGGCATTAGCTCGCTTCAAAAAACTTTACGCAATGCTCTTATT
CCGACTGAGACGACACAACAGTTTATCGTTAAGAATGGCATCATCAAAGAAGATGAATTA
CGCGGAGAAAACCGCCAGATCCTGAAAGACATTATGGACGATTATTACCGTGGGTTCATC
TCCGAGACGTTGTCATCGATCGATGACATCGACTGGACGTCACTTTTTGAAAAAATGGAG
ATCCAGTTAAAGAACGGTGACAATAAGGATACATTGATCAAAGAACAGACCGAGTACCGT
AAAGCGATTCATAAAAAGTTTGCGAACGATGATCGCTTCAAGAATATGTTTTCTGCGAAA
TTAATTTCCGACATTTTACCTGAATTTGTTATTCATAATAACAACTACTCGGCGTCTGAG
AAAGAGGAGAAAACCCAAGTGATTAAACTTTTTTCACGTTTCGCAACGTCGTTCAAAGAC
TATTTTAAAAATCGTGCTAATTGCTTTAGCGCGGATGACATCAGCTCTAGTTCATGTCAT
CGCATTGTCAACGATAATGCTGAGATCTTTTTCAGTAATGCGTTAGTGTACCGTCGTATT
GTGAAGTCCTTATCTAATGATGATATCAATAAGATCAGCGGGGATATGAAGGACTCACTT
AAGGAGATGAGCTTGGAGGAAATCTATTCCTATGAGAAGTATGGTGAGTTTATTACGCAA
GAAGGAATTAGCTTTTACAACGATATCTGTGGAAAGGTGAATTCGTTTATGAATTTGTAT
TGCCAGAAAAATAAGGAGAACAAGAACCTTTATAAATTGCAAAAGTTACACAAGCAAATC
CTGTGCATTGCAGATACTTCCTACGAGGTGCCTTACAAGTTTGAATCCGACGAAGAGGTC
TACCAATCTGTAAACGGTTTCTTAGATAATATTAGTTCCAAGCATATTGTGGAGCGCCTT
CGTAAAATTGGCGATAATTACAACGGTTACAATTTAGACAAAATTTACATTGTCAGTAAA
TTCTACGAGTCCGTATCTCAAAAGACGTATCGTGATTGGGAGACTATCAATACGGCCCTG
GAGATCCACTACAACAATATCTTGCCCGGTAATGGTAAGTCGAAGGCCGATAAAGTTAAG
AAAGCGGTGAAAAATGACTTACAGAAGTCAATCACCGAAATTAACGAATTGGTGTCCAAT
TATAAATTGTGTTCAGATGATAATATCAAAGCCGAGACCTACATTCATGAGATTTCCCAT
ATCTTAAATAATTTCGAGGCGCAAGAGCTTAAGTATAACCCAGAAATCCACCTGGTAGAA
TCTGAGTTGAAGGCGTCAGAGTTAAAAAATGTTTTAGATGTCATTATGAACGCGTTTCAC
TGGTGCTCCGTATTTATGACGGAGGAATTAGTAGATAAAGACAACAATTTCTATGCCGAA
CTTGAGGAAATCTATGATGAGATCTATCCCGTCATTAGCCTGTATAACTTGGTCCGCAAC
TATGTTACCCAAAAACCGTACAGTACCAAGAAGATTAAGCTGAATTTCGGCATTCCTACA
CTGGCTGATGGTTGGAGTAAATCGAAGGAATATTCGAATAACGCGATTATCTTGATGCGC
GACAACTTATACTATTTGGGGATCTTTAACGCCAAAAACAAACCGGATAAGAAGATTATT
GAGGGAAACACATCAGAGAACAAAGGCGACTACAAAAAAATGATTTACAACTTGTTACCG
GGGCCTAACAAAATGATCCCGAAGGTGTTCTTATCCAGTAAAACAGGCGTTGAGACCTAC
AAACCTTCCGCATACATCCTGGAAGGGTATAAGCAGAACAAGCACATTAAGTCCAGCAAG
GATTTCGATATTACCTTCTGTCATGATTTAATTGACTATTTCAAGAACTGTATTGCAATC
CACCCCGAGTGGAAGAACTTCGGATTCGACTTCTCAGATACGAGCACATATGAGGACATC
TCGGGGTTCTATCGTGAAGTAGAACTGCAGGGATATAAAATTGATTGGACATATATTTCC
GAAAAAGACATCGACCTTTTACAAGAGAAGGGTCAACTTTACTTGTTCCAAATTTACAAT
AAAGACTTCTCAAAAAAAAGCACGGGTAACGATAATTTACACACTATGTATTTAAAGAAC
CTTTTCTCGGAAGAGAATTTAAAGGATATCGTATTGAAGTTGAATGGAGAAGCGGAGATC
TTCTTCCGTAAGTCCAGTATTAAAAACCCTATTATTCACAAGAAGGGATCGATTTTAGTT
AACCGCACATACGAGGCCGAAGAGAAGGACCAATTTGGGAACATTCAAATTGTCCGCAAA
AACATCCCTGAGAACATTTATCAAGAGCTTTATAAGTACTTTAACGATAAGTCCGATAAG
GAATTGTCAGATGAGGCGGCAAAGTTGAAGAATGTCGTGGGGCATCATGAAGCTGCCACC
AACATTGTGAAGGACTACCGCTACACTTACGACAAATACTTCCTGCACATGCCCATTACG
ATCAATTTTAAGGCCAATAAGACAGGCTTTATTAACGACCGTATTCTTCAATATATCGCT
AAGGAGAAGGACCTTCATGTGATTGGGATCGACCGCGGAGAACGTAATTTAATTTATGTG
TCCGTCATCGATACGTGTGGAAATATCGTGGAACAGAAATCATTCAATATCGTGAATGGC
TATGATTACCAGATCAAATTAAAACAGCAGGAGGGCGCTCGCCAAATTGCGCGTAAGGAA
TGGAAAGAGATCGGAAAAATCAAAGAAATCAAAGAAGGATATTTGTCATTGGTGATCCAT
GAGATTTCAAAAATGGTAATTAAATATAATGCAATTATCGCAATGGAAGACCTGTCCTAT
GGTTTTAAGAAGGGTCGTTTCAAGGTAGAACGCCAAGTGTATCAAAAGTTCGAGACGATG
CTGATCAATAAGCTGAATTATCTTGTGTTTAAGGACATTAGCATCACGGAAAATGGAGGG
CTGTTGAAAGGCTATCAACTGACGTATATCCCTGACAAGCTGAAAAATGTTGGCCATCAG
TGCGGGTGCATTTTCTACGTCCCCGCGGCGTATACAAGCAAGATCGATCCTACTACGGGA
TTCGTAAATATTTTTAAATTCAAAGACTTAACCGTGGACGCCAAGCGCGAATTCATTAAG
AAGTTTGATAGCATTCGCTACGATTCAGAAAAAAATCTTTTCTGTTTTACGTTCGATTAC
AACAATTTTATCACCCAGAACACAGTGATGAGCAAGTCATCCTGGTCTGTCTATACCTAC
GGTGTCCGTATCAAACGCCGCTTCGTCAACGGACGCTTCTCTAATGAATCTGATACCATT
GACATCACCAAGGACATGGAAAAGACACTTGAGATGACAGATATTAACTGGCGTGACGGA
CATGACCTGCGTCAGGACATCATCGATTATGAGATTGTTCAGCATATCTTCGAGATCTTC
CGCCTGACAGTACAAATGCGCAATTCACTGTCAGAACTTGAAGACCGCGACTATGACCGC
CTGATCTCTCCAGTATTAAATGAGAACAATATCTTTTATGACAGTGCTAAGGCCGGCGAT
GCCCTTCCGAAAGATGCTGATGCTAACGGAGCTTATTGTATTGCATTAAAGGGTCTTTAT
GAGATCAAGCAAATTACCGAGAATTGGAAGGAGGATGGCAAATTCTCGCGCGACAAACTG
AAAATCAGTAACAAGGACTGGTTCGATTTTATTCAGAATAAACGTTACCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 74
AGCCCAGCGGCTAAAAAAAAGAAACTGGATGGCAGCGTGGATATGAATAACGGAACGAAC
AACTTCCAGAACTTCATCGGCATCAGTTCTTTACAAAAAACCCTGCGTAACGCCCTTATT
CCGACTGAGACAACACAACAGTTCATCGTTAAAAACGGAATTATCAAAGAGGACGAGTTG
CGCGGCGAGAATCGCCAAATTTTGAAAGATATTATGGACGACTATTATCGTGGTTTTATT
TCAGAAACACTGAGTTCGATTGACGATATCGATTGGACGAGCCTGTTTGAGAAAATGGAA
ATCCAGTTGAAAAATGGCGATAATAAAGACACTTTAATCAAAGAACAAACCGAGTATCGT
AAAGCGATCCATAAAAAGTTCGCTAATGACGATCGTTTTAAGAATATGTTCAGTGCGAAA
CTGATTTCAGACATTTTGCCCGAGTTCGTGATCCATAATAACAACTATTCCGCCTCGGAA
AAGGAAGAAAAAACCCAGGTGATTAAGCTGTTCAGTCGCTTCGCAACATCTTTCAAGGAT
TATTTCAAGAATCGCGCGAATTGCTTCAGTGCGGACGATATTTCTAGTTCAAGCTGCCAT
CGTATCGTTAATGATAACGCGGAGATTTTTTTTAGCAATGCTCTGGTGTACCGCCGCATT
GTTAAGTCACTGTCCAACGATGATATTAACAAGATCTCAGGAGACATGAAAGACTCGCTT
AAAGAGATGAGTCTGGAAGAGATCTATTCTTATGAGAAGTATGGCGAGTTTATTACCCAA
GAAGGAATCTCATTCTACAATGATATTTGTGGAAAGGTGAACAGCTTTATGAATCTTTAC
TGCCAAAAAAACAAGGAGAATAAGAATCTTTACAAACTTCAGAAGTTACATAAACAGATT
TTGTGTATTGCGGATACGTCTTATGAAGTCCCCTACAAATTTGAATCGGATGAAGAGGTA
TACCAAAGTGTGAACGGATTCTTGGACAATATTTCTTCTAAACATATTGTTGAACGCTTA
CGTAAGATCGGGGATAACTACAATGGCTACAATCTTGACAAAATCTACATTGTTAGCAAA
TTCTACGAGAGTGTCAGCCAAAAGACGTACCGCGATTGGGAAACAATTAATACTGCGCTT
GAGATTCACTATAATAACATTTTACCAGGCAACGGCAAGTCCAAGGCGGATAAAGTTAAA
AAAGCTGTTAAAAACGATTTGCAAAAATCTATCACAGAAATTAACGAGTTAGTTAGTAAC
TACAAACTGTGCTCCGATGACAACATTAAGGCTGAGACGTATATCCATGAGATCTCTCAC
ATCTTAAACAATTTTGAAGCTCAAGAACTTAAGTACAATCCGGAAATCCACCTGGTGGAA
TCCGAGCTGAAGGCTAGCGAACTGAAGAACGTATTGGACGTGATCATGAACGCGTTCCAC
TGGTGTTCTGTCTTTATGACGGAAGAGCTTGTCGACAAAGATAATAACTTTTACGCGGAA
CTTGAGGAAATTTACGATGAGATTTACCCAGTTATTTCATTGTATAACCTTGTCCGTAAT
TACGTGACCCAAAAGCCTTATAGTACGAAAAAAATCAAATTAAATTTTGGAATCCCAACA
CTGGCTGACGGTTGGAGCAAATCTAAGGAGTATTCTAATAACGCAATCATCTTAATGCGT
GACAACCTGTATTATTTGGGTATCTTCAATGCCAAAAATAAGCCTGACAAAAAGATTATC
GAAGGAAATACTTCGGAGAATAAGGGGGATTACAAAAAAATGATTTACAATTTGCTGCCC
GGGCCGAACAAGATGATCCCCAAAGTGTTCTTATCCTCGAAGACTGGTGTAGAAACATAC
AAGCCAAGCGCATACATTCTGGAGGGTTACAAGCAAAACAAACACATCAAATCTTCAAAA
GACTTTGACATTACATTTTGCCATGATCTTATTGACTACTTCAAAAACTGCATTGCTATT
CACCCCGAGTGGAAGAACTTTGGGTTTGACTTCAGCGACACGTCTACGTATGAGGACATC
TCCGGGTTCTACCGTGAAGTTGAGTTACAAGGGTATAAGATTGACTGGACGTATATTTCA
GAGAAAGATATCGATCTTTTGCAGGAAAAGGGCCAGTTATATTTATTCCAGATTTACAAC
AAGGACTTTAGTAAGAAGTCAACAGGAAATGACAACTTGCATACGATGTATTTGAAAAAT
CTTTTTTCTGAGGAAAATCTTAAGGACATCGTACTGAAATTGAATGGCGAGGCTGAAATC
TTCTTCCGTAAATCCTCCATTAAGAATCCCATTATCCACAAAAAGGGGTCTATCCTGGTG
AATCGTACCTACGAGGCAGAGGAGAAGGATCAATTCGGAAATATTCAGATTGTTCGTAAG
AACATCCCCGAGAACATTTATCAAGAATTGTATAAGTACTTTAATGACAAATCTGACAAA
GAGTTATCCGACGAAGCTGCGAAACTGAAAAACGTTGTTGGTCACCACGAGGCCGCCACT
AATATCGTAAAAGACTACCGTTATACCTATGACAAGTACTTTTTGCACATGCCGATCACT
ATCAACTTCAAGGCGAATAAGACGGGCTTCATTAACGATCGTATCCTGCAATACATCGCC
AAGGAGAAGGACCTTCACGTCATTGGGATTGACCGTGGTGAGCGTAACCTGATTTATGTA
AGCGTCATTGATACCTGCGGTAATATCGTCGAACAGAAAAGTTTCAACATTGTAAATGGA
TATGACTATCAGATCAAACTTAAGCAGCAGGAGGGTGCACGCCAGATTGCCCGCAAGGAA
TGGAAGGAGATTGGGAAGATTAAGGAAATTAAAGAAGGTTACTTATCACTGGTTATTCAC
GAGATCAGTAAAATGGTAATCAAATATAACGCGATCATTGCCATGGAGGATCTGAGCTAT
GGCTTTAAAAAGGGCCGTTTCAAAGTCGAGCGCCAGGTATATCAAAAGTTTGAAACAATG
CTGATTAACAAATTAAACTATCTGGTTTTCAAAGATATTTCGATCACTGAAAATGGCGGG
CTGTTGAAGGGATACCAACTTACATACATCCCTGACAAACTGAAAAATGTCGGTCACCAA
TGTGGATGTATCTTTTATGTACCAGCAGCGTATACGAGCAAAATCGATCCAACTACGGGT
TTTGTGAACATCTTTAAGTTCAAGGATTTGACAGTAGATGCCAAACGCGAGTTCATTAAA
AAATTTGATTCAATTCGCTACGATTCAGAGAAAAATCTTTTTTGTTTCACGTTCGATTAC
AATAATTTCATTACGCAGAACACAGTAATGTCAAAGTCAAGCTGGTCGGTCTACACGTAT
GGAGTCCGTATTAAACGTCGTTTTGTAAACGGCCGTTTCTCAAATGAATCAGATACAATT
GATATTACGAAGGATATGGAGAAGACATTAGAGATGACTGACATTAACTGGCGCGACGGA
CATGATCTTCGTCAGGACATTATTGATTATGAGATTGTACAGCATATCTTTGAGATCTTC
CGCCTGACCGTTCAGATGCGCAATTCGTTGTCCGAGTTAGAAGACCGCGATTACGACCGT
TTAATCAGTCCCGTCTTAAACGAAAATAACATCTTCTACGATTCAGCCAAGGCAGGCGAT
GCCTTGCCAAAGGATGCTGACGCAAATGGCGCATACTGTATTGCGTTGAAAGGCCTTTAT
GAAATCAAGCAAATTACCGAAAACTGGAAAGAAGACGGAAAATTCTCCCGTGATAAGTTG
AAAATCTCTAATAAGGATTGGTTCGATTTCATCCAAAATAAACGCTATTTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 75
AGCCCAGCGGCTAAAAAAAAGAAACTGGATGGCAGCGTGGATATGAACAACGGAACTAAT
AATTTCCAAAATTTTATAGGCATCTCTTCTTTACAGAAGACTCTTCGTAACGCCCTAATC
CCGACTGAGACCACACAACAATTCATAGTGAAAAATGGGATCATTAAAGAAGACGAGCTG
CGTGGGGAGAACAGGCAGATCCTAAAAGACATAATGGACGATTATTATAGAGGGTTCATC
TCAGAGACATTATCTAGCATCGACGACATTGACTGGACCTCCCTGTTTGAAAAAATGGAA
ATCCAGCTGAAGAATGGTGACAATAAAGACACATTAATAAAAGAACAAACAGAGTACAGG
AAAGCCATCCACAAGAAGTTCGCAAACGATGACAGATTCAAAAATATGTTCAGTGCGAAG
CTAATATCCGACATCTTACCAGAGTTTGTAATACACAATAACAATTACAGCGCGAGCGAA
AAGGAAGAGAAAACGCAAGTAATTAAGCTTTTTAGTAGGTTCGCTACCTCTTTCAAAGAT
TACTTCAAAAATCGTGCTAACTGCTTCTCAGCCGACGACATATCTTCAAGTTCCTGTCAC
CGTATCGTGAATGATAACGCTGAGATATTCTTCTCAAACGCCCTTGTATACCGTAGGATC
GTAAAGTCCTTATCTAACGATGATATAAACAAGATCAGTGGAGACATGAAAGACAGCCTT
AAAGAGATGTCTCTAGAAGAAATTTACTCCTATGAAAAGTATGGGGAGTTTATAACACAG
GAGGGGATCAGCTTCTACAACGACATCTGCGGAAAGGTGAACAGTTTCATGAATCTTTAC
TGCCAGAAGAATAAAGAGAACAAAAATCTTTATAAGCTTCAAAAGTTGCACAAACAAATA
CTGTGCATTGCCGATACATCATATGAGGTCCCCTATAAGTTCGAATCTGATGAGGAAGTT
TATCAATCTGTTAACGGCTTTCTAGACAATATCAGCTCAAAACACATCGTAGAAAGACTG
AGGAAAATAGGTGATAATTATAATGGATACAACTTGGATAAAATATATATAGTCTCTAAA
TTTTACGAGTCAGTATCCCAGAAAACGTATAGGGATTGGGAGACCATCAACACGGCGTTA
GAGATTCATTACAATAACATCTTACCGGGAAACGGAAAAAGTAAGGCGGACAAAGTAAAG
AAAGCCGTTAAAAATGACTTACAAAAGAGTATAACAGAAATAAACGAACTAGTAAGCAAC
TACAAGCTTTGTTCCGATGATAATATCAAGGCCGAGACATATATCCATGAGATCTCCCAC
ATTCTAAACAATTTCGAAGCGCAAGAACTTAAATATAATCCCGAAATCCACCTGGTGGAA
AGTGAACTAAAGGCTAGTGAGTTAAAGAACGTTCTTGATGTTATCATGAACGCCTTCCAT
TGGTGCTCTGTTTTTATGACCGAGGAGTTGGTTGATAAAGATAATAATTTCTACGCTGAA
TTAGAGGAGATATACGACGAAATCTACCCAGTGATTTCACTATACAACTTGGTCAGGAAC
TATGTTACACAAAAGCCGTACAGCACTAAGAAAATTAAGCTAAATTTCGGTATCCCCACG
TTAGCCGACGGGTGGAGCAAGTCCAAAGAATATTCCAACAATGCGATTATTTTAATGCGT
GACAATCTTTATTACCTTGGCATCTTCAATGCCAAAAACAAACCTGACAAAAAGATTATA
GAAGGTAATACGTCCGAGAACAAAGGCGATTACAAGAAGATGATTTATAACCTACTGCCC
GGACCAAACAAAATGATCCCCAAAGTTTTTCTTAGTTCTAAAACCGGCGTAGAGACGTAT
AAACCTTCTGCCTATATCTTAGAGGGATATAAGCAGAACAAACATATCAAATCTTCCAAG
GACTTTGATATTACATTCTGCCACGATTTAATTGACTACTTCAAAAATTGCATAGCGATA
CATCCGGAGTGGAAGAACTTTGGCTTCGACTTCAGTGATACATCCACCTATGAGGATATA
TCAGGCTTCTATCGTGAGGTCGAATTGCAAGGGTACAAAATCGATTGGACGTATATATCC
GAGAAAGACATAGACCTTCTTCAAGAAAAGGGGCAGTTATATTTATTCCAAATATACAAC
AAGGACTTCAGTAAGAAGTCAACAGGTAATGACAACTTACACACCATGTACTTGAAAAAT
TTATTTTCTGAAGAAAACCTAAAGGACATTGTACTAAAACTGAACGGGGAGGCAGAAATT
TTTTTTAGAAAGAGCAGCATAAAAAACCCAATAATTCATAAGAAAGGAAGCATTTTAGTT
AATAGGACGTACGAGGCAGAGGAAAAGGACCAGTTTGGCAATATCCAGATCGTAAGGAAA
AATATTCCTGAAAACATATATCAGGAACTATATAAATACTTTAACGACAAATCCGACAAA
GAATTATCCGACGAGGCTGCAAAGCTGAAGAACGTCGTAGGGCACCATGAGGCAGCGACT
AATATTGTGAAAGACTATAGGTATACATACGACAAATACTTTCTGCACATGCCCATCACG
ATTAACTTCAAGGCGAACAAGACGGGATTCATTAACGACCGTATATTACAATATATTGCT
AAGGAGAAAGATCTGCATGTAATAGGTATCGACAGAGGCGAACGTAATTTAATCTACGTG
TCCGTCATCGACACGTGCGGGAACATCGTAGAGCAAAAGAGTTTTAATATAGTAAATGGC
TATGATTACCAAATTAAGCTAAAGCAGCAAGAAGGAGCAAGACAGATAGCTAGGAAAGAA
TGGAAGGAGATAGGAAAAATAAAGGAGATCAAGGAGGGGTATCTTAGCCTAGTAATTCAT
GAAATATCTAAGATGGTTATCAAATACAACGCTATCATAGCGATGGAAGACTTATCTTAT
GGTTTCAAGAAAGGAAGGTTCAAAGTAGAGCGTCAAGTTTATCAAAAGTTCGAAACGATG
TTGATTAATAAACTAAACTATTTGGTATTTAAAGATATATCTATCACCGAGAATGGTGGT
CTACTAAAGGGTTACCAGCTTACATACATACCGGACAAACTTAAAAACGTCGGACATCAG
TGTGGATGCATTTTCTACGTTCCAGCTGCATATACCAGCAAGATCGACCCAACGACTGGG
TTCGTAAATATTTTTAAATTCAAGGATTTGACTGTCGACGCCAAAAGAGAGTTCATAAAA
AAGTTCGATTCAATTAGGTACGACAGCGAAAAGAATTTGTTCTGCTTTACTTTTGACTAT
AACAATTTCATTACTCAGAACACTGTAATGTCTAAGTCCTCTTGGTCAGTCTATACTTAT
GGCGTTCGTATCAAACGTAGATTTGTTAACGGTAGATTCTCAAATGAAAGTGATACAATA
GATATCACGAAAGATATGGAGAAAACATTAGAAATGACAGACATAAACTGGAGAGACGGA
CATGACTTGAGACAGGACATTATTGACTACGAGATCGTGCAGCACATCTTTGAGATCTTT
CGTTTGACCGTACAAATGCGTAACAGTTTATCTGAGCTTGAGGACAGGGACTACGATAGA
TTGATATCACCTGTATTAAATGAGAATAACATCTTCTATGATTCCGCAAAAGCAGGCGAC
GCTCTACCCAAAGACGCTGATGCGAACGGTGCTTATTGCATAGCTTTAAAGGGTTTGTAT
GAGATCAAACAGATAACAGAAAATTGGAAGGAAGATGGTAAGTTCTCCCGTGACAAGCTT
AAAATATCAAATAAGGACTGGTTCGATTTTATACAGAATAAGCGTTATTAAAACGTCCGG
CAGCGACCAAAAAAGCCGGCCAGGCGAAGAAAAAAAAAGCGTCAGGTAGCGGCGCAGGCA
GCCCGAAAAAGAAACGTAAAGTCGAGGATCCGAAAAAGAAACGTAAGGTTATTCCGGGCT
AA
SEQ ID NO: 76
AGCCCAGCGGCTAAAAAAAAGAAACTGGATGGCAGCGTGGATATGAACAATGGAACTAAT
AACTTCCAGAATTTCATTGGTATCTCCTCTTTACAAAAAACTCTAAGAAACGCCCTAATT
CCGACTGAAACTACACAGCAATTCATCGTCAAAAACGGGATCATTAAGGAGGATGAGTTG
AGGGGTGAAAATCGTCAAATTCTTAAAGACATCATGGACGACTACTACAGGGGGTTCATC
AGCGAGACGTTATCTAGTATAGACGATATAGACTGGACTTCACTGTTCGAGAAGATGGAA
ATCCAATTAAAAAATGGGGACAATAAAGATACACTTATAAAGGAACAGACAGAGTATAGA
AAGGCAATACACAAAAAGTTTGCCAACGACGATCGTTTCAAGAACATGTTTAGTGCTAAA
TTGATTTCAGATATTCTGCCGGAATTTGTTATTCACAACAATAATTATAGCGCCAGTGAG
AAAGAAGAAAAAACGCAGGTTATCAAACTGTTCAGTCGTTTCGCTACATCTTTTAAGGAT
TACTTTAAAAACCGTGCAAATTGTTTTTCAGCCGACGATATTAGTAGCAGCTCTTGTCAC
CGTATTGTTAATGATAATGCGGAGATTTTCTTTTCAAACGCATTGGTCTACAGGAGGATA
GTCAAGTCCCTTTCAAATGACGACATTAATAAGATCTCAGGTGACATGAAAGATTCCTTA
AAGGAAATGTCCCTGGAAGAGATCTATTCCTATGAAAAGTACGGTGAGTTCATTACTCAA
GAGGGTATAAGCTTTTACAATGACATATGTGGTAAGGTTAATAGCTTTATGAACCTGTAT
TGCCAGAAGAACAAAGAAAATAAGAATCTGTATAAGTTGCAAAAGCTACACAAACAAATT
TTGTGCATTGCCGATACATCATACGAGGTGCCATACAAATTCGAGAGCGATGAGGAGGTT
TATCAGAGCGTGAATGGATTCCTGGACAATATTAGTAGTAAGCATATCGTGGAAAGGCTT
AGAAAGATAGGTGACAATTACAATGGCTACAATCTGGATAAAATCTACATCGTCTCAAAA
TTCTATGAAAGTGTATCCCAGAAGACGTACCGTGATTGGGAAACTATCAACACCGCTCTG
GAGATACATTACAACAATATACTTCCCGGAAACGGCAAGTCAAAAGCCGACAAAGTCAAA
AAAGCGGTCAAGAACGATTTACAAAAGTCTATCACTGAAATTAATGAATTAGTTAGTAAT
TACAAACTGTGTAGTGATGATAATATTAAGGCAGAGACTTACATACACGAAATTTCACAC
ATTTTAAACAACTTCGAGGCACAGGAACTTAAATATAATCCTGAAATTCACCTGGTTGAA
AGTGAATTGAAAGCCAGCGAGCTAAAGAACGTTTTGGACGTAATCATGAACGCATTCCAC
TGGTGCTCTGTCTTTATGACAGAGGAACTAGTGGATAAGGACAATAATTTTTATGCGGAG
CTGGAGGAAATATACGATGAGATATATCCCGTAATATCATTATATAATCTGGTAAGAAAC
TATGTGACTCAAAAGCCGTATAGCACCAAGAAAATTAAACTTAATTTCGGCATACCCACT
TTAGCGGACGGCTGGTCAAAATCCAAAGAGTATAGTAATAATGCCATCATCCTGATGCGT
GACAACCTGTACTATTTAGGTATATTTAACGCCAAAAATAAACCCGACAAAAAGATTATA
GAGGGCAACACCTCAGAGAACAAAGGTGATTATAAGAAGATGATTTACAACCTTTTACCC
GGTCCTAATAAGATGATTCCCAAAGTCTTTCTATCTAGCAAAACTGGTGTTGAAACATAC
AAACCCTCAGCTTATATTTTAGAAGGGTATAAGCAGAATAAGCATATTAAAAGCTCCAAA
GATTTCGATATTACCTTTTGCCATGACTTGATAGACTATTTCAAAAATTGTATTGCCATT
CACCCTGAATGGAAAAACTTCGGATTTGACTTCTCTGACACATCCACCTACGAAGACATT
TCAGGTTTTTACAGGGAAGTCGAGCTACAGGGTTATAAAATTGATTGGACATACATCAGC
GAGAAAGATATTGACCTACTTCAAGAAAAAGGGCAGCTATACCTGTTCCAGATATACAAT
AAAGACTTCAGTAAAAAAAGCACCGGGAACGATAATCTTCACACAATGTACTTAAAAAAT
TTATTTAGTGAAGAGAATCTGAAGGATATAGTGCTGAAGTTAAACGGGGAGGCAGAGATA
TTTTTTAGAAAATCTAGTATTAAGAATCCGATCATCCACAAGAAGGGTTCTATCCTTGTT
AATAGGACTTATGAGGCAGAAGAAAAAGACCAATTCGGCAACATACAAATTGTCCGTAAA
AATATCCCTGAGAACATTTATCAGGAACTATACAAGTACTTCAATGATAAAAGCGACAAG
GAGCTGAGCGACGAGGCTGCTAAGTTAAAGAATGTGGTGGGCCACCATGAGGCAGCAACG
AATATTGTGAAGGACTATCGTTATACCTACGATAAATACTTTCTTCATATGCCGATCACC
ATTAATTTCAAGGCAAACAAAACTGGCTTCATTAACGATCGTATCTTACAATATATCGCA
AAAGAGAAAGACCTTCACGTTATCGGGATCGATAGAGGCGAGCGTAACCTAATTTATGTT
TCTGTGATAGACACCTGTGGGAACATAGTCGAACAGAAATCATTTAATATTGTTAACGGC
TACGATTATCAGATAAAGTTGAAGCAACAAGAGGGTGCACGTCAAATAGCAAGGAAAGAA
TGGAAAGAAATAGGCAAGATTAAAGAAATAAAAGAAGGTTATTTATCCCTTGTAATACAC
GAAATTAGCAAAATGGTGATTAAATATAATGCGATCATTGCCATGGAGGATCTTTCTTAC
GGCTTCAAAAAGGGGAGATTCAAAGTCGAGAGGCAGGTGTATCAGAAGTTTGAGACCATG
CTAATCAATAAACTAAATTATCTAGTATTCAAAGACATAAGCATCACCGAAAATGGCGGC
TTGTTGAAGGGTTATCAATTGACCTACATCCCAGATAAACTAAAAAACGTAGGGCATCAA
TGCGGATGTATATTTTACGTTCCAGCCGCATACACTTCCAAAATCGATCCAACTACGGGT
TTTGTGAACATCTTCAAATTCAAAGACTTGACTGTCGATGCTAAGAGGGAGTTTATCAAG
AAATTTGACTCCATTAGATACGACAGTGAGAAGAATCTGTTCTGTTTTACCTTTGATTAT
AACAACTTTATAACTCAAAACACAGTCATGAGTAAGTCATCTTGGTCAGTGTATACGTAT
GGTGTGAGGATTAAAAGGAGGTTTGTTAACGGGAGATTTTCCAATGAAAGTGATACAATA
GATATAACCAAGGACATGGAAAAGACTCTTGAAATGACCGACATTAACTGGAGAGATGGC
CACGACTTACGTCAAGATATAATCGATTACGAGATAGTGCAACATATCTTTGAGATATTT
AGGCTTACTGTCCAAATGCGTAACTCATTAAGTGAGTTGGAGGACAGGGATTACGATAGG
CTAATAAGTCCTGTTCTTAACGAAAACAATATATTCTACGATTCAGCAAAGGCGGGAGAC
GCCCTGCCCAAGGACGCGGATGCTAACGGCGCATACTGTATTGCCCTGAAAGGCTTGTAC
GAGATAAAACAGATCACGGAGAACTGGAAAGAAGATGGAAAATTCAGTCGTGACAAGTTA
AAAATTAGTAACAAAGACTGGTTCGACTTTATTCAGAACAAGAGATATCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 77
AGCCCAGCGGCTAAAAAAAAGAAACTGGATGGCAGCGTGGATATGAACAACGGAACCAAT
AACTTTCAAAACTTTATAGGCATCTCCAGTCTACAGAAGACACTACGTAACGCTTTGATA
CCAACTGAGACCACGCAGCAGTTTATCGTCAAGAACGGTATTATAAAGGAAGACGAGCTA
AGGGGGGAAAACCGTCAGATCTTAAAGGACATCATGGATGACTACTACAGAGGCTTCATA
AGTGAGACTTTGTCTAGTATAGACGACATCGACTGGACCAGTTTATTTGAGAAGATGGAA
ATTCAGTTAAAGAACGGGGACAATAAAGACACACTAATTAAAGAGCAGACCGAATACAGA
AAAGCTATACACAAAAAGTTTGCCAACGATGATAGATTCAAAAATATGTTTTCAGCAAAA
TTGATTTCCGACATATTGCCAGAATTCGTAATCCATAATAACAATTATTCTGCAAGTGAG
AAGGAAGAGAAGACCCAAGTAATCAAGCTGTTTTCCCGTTTTGCTACGAGTTTCAAAGAT
TATTTCAAGAATAGGGCTAATTGTTTCTCCGCGGACGACATAAGTAGCAGTTCCTGTCAC
AGGATTGTGAACGATAATGCTGAGATATTTTTTTCCAATGCCCTAGTGTATAGGAGAATA
GTTAAAAGCTTAAGCAACGACGATATCAATAAAATTTCAGGGGACATGAAGGACAGCTTA
AAGGAAATGAGTTTGGAGGAGATTTACAGTTATGAAAAATACGGAGAGTTTATAACTCAG
GAAGGCATCTCTTTCTATAATGATATCTGTGGGAAGGTAAACTCCTTCATGAATTTATAT
TGCCAGAAGAATAAGGAAAACAAAAATCTTTACAAGCTTCAAAAGTTACATAAGCAGATC
TTATGTATTGCCGACACGAGTTATGAAGTGCCTTATAAATTCGAGAGTGATGAGGAAGTG
TATCAGTCTGTTAACGGATTCCTAGATAATATAAGTTCCAAACATATAGTCGAGAGGCTG
AGGAAGATTGGCGATAACTATAATGGATATAATCTTGACAAAATCTATATAGTCTCTAAA
TTTTATGAAAGCGTCAGCCAGAAGACATATAGAGATTGGGAAACTATAAACACAGCCCTT
GAAATACATTACAATAACATCCTACCCGGCAATGGTAAGTCTAAGGCAGACAAAGTTAAA
AAAGCAGTAAAGAATGACTTACAGAAGTCAATCACGGAGATAAATGAGTTGGTCAGTAAC
TACAAATTATGCTCCGACGATAATATTAAGGCCGAAACATATATACACGAGATAAGTCAT
ATATTAAACAATTTCGAAGCCCAGGAGTTAAAATATAACCCTGAAATTCATCTGGTCGAA
AGTGAGTTAAAGGCCAGTGAGTTAAAGAATGTACTTGACGTAATTATGAATGCTTTTCAT
TGGTGCTCCGTGTTCATGACCGAGGAGTTAGTAGATAAAGACAATAACTTTTACGCCGAA
CTTGAAGAGATATACGACGAGATTTATCCGGTAATCAGCTTGTACAACTTAGTTAGAAAT
TATGTAACACAGAAGCCTTACTCTACTAAAAAAATAAAACTGAACTTTGGTATCCCAACT
CTTGCAGATGGTTGGAGTAAAAGCAAGGAATATAGCAACAATGCGATCATCTTGATGAGA
GACAACTTGTACTATTTGGGAATCTTCAACGCGAAAAATAAACCCGACAAAAAAATCATC
GAAGGGAATACCTCTGAGAATAAAGGTGACTATAAGAAAATGATTTACAATCTACTTCCT
GGTCCTAATAAAATGATCCCGAAAGTGTTTCTTAGTTCTAAGACTGGTGTCGAGACGTAC
AAACCTAGCGCGTACATCTTAGAAGGGTACAAGCAGAATAAACACATCAAATCAAGCAAA
GACTTCGATATTACTTTTTGCCATGACTTGATAGACTACTTTAAAAACTGCATAGCAATC
CACCCGGAGTGGAAAAACTTTGGCTTTGATTTCTCTGACACCTCTACATATGAGGACATA
TCTGGTTTTTACCGTGAGGTTGAATTGCAGGGATACAAAATTGACTGGACTTACATATCT
GAAAAAGATATCGATCTATTGCAGGAGAAAGGCCAGCTTTACCTTTTCCAGATCTATAAT
AAGGACTTCTCTAAGAAGTCTACAGGGAATGATAATTTGCACACTATGTACTTAAAAAAT
CTGTTTTCCGAGGAAAACTTGAAAGACATTGTTTTAAAGTTGAACGGAGAAGCTGAAATA
TTTTTCAGAAAGAGCTCCATAAAAAACCCGATCATTCATAAGAAGGGATCTATCCTGGTT
AACAGAACGTACGAAGCGGAAGAAAAAGACCAATTCGGAAACATTCAAATTGTTAGAAAG
AATATCCCTGAGAACATCTACCAGGAGTTATATAAGTATTTTAATGATAAGTCAGATAAG
GAACTATCTGACGAAGCGGCGAAGCTTAAAAATGTTGTAGGACACCATGAGGCTGCTACA
AATATAGTCAAGGACTACCGTTATACCTACGATAAGTACTTTCTACACATGCCCATTACC
ATCAATTTTAAAGCTAATAAAACGGGTTTTATCAACGATCGTATCCTACAATATATTGCG
AAAGAGAAGGATTTGCATGTCATTGGCATTGATAGAGGTGAGAGGAACCTAATATACGTA
TCCGTGATTGATACGTGCGGGAACATAGTTGAACAGAAATCATTTAATATAGTTAATGGG
TACGACTATCAGATTAAGCTAAAGCAACAAGAAGGCGCCAGGCAAATTGCCCGTAAAGAA
TGGAAAGAGATCGGGAAGATCAAGGAAATAAAAGAAGGATACCTTTCCCTGGTCATCCAT
GAAATTAGCAAAATGGTGATTAAGTACAATGCCATAATCGCGATGGAGGACTTAAGCTAC
GGGTTCAAAAAGGGGAGGTTTAAGGTGGAGAGGCAAGTGTACCAGAAATTTGAGACCATG
CTAATCAACAAACTGAACTACCTAGTTTTTAAGGACATTTCAATTACAGAGAATGGAGGA
CTTTTAAAGGGTTACCAACTAACGTATATACCAGATAAGTTGAAAAATGTCGGTCACCAG
TGTGGCTGCATCTTTTACGTTCCCGCCGCTTATACATCTAAAATTGATCCAACCACAGGC
TTTGTAAATATCTTTAAATTCAAAGATTTAACTGTGGATGCAAAAAGAGAGTTTATCAAG
AAATTCGATAGCATTCGTTATGATAGCGAGAAGAACCTGTTCTGCTTTACTTTCGACTAT
AACAACTTTATAACTCAAAACACCGTGATGTCAAAAAGCTCATGGTCAGTCTACACCTAT
GGTGTAAGGATTAAAAGGCGTTTCGTGAATGGGAGATTCTCCAATGAAAGTGACACGATC
GACATAACAAAGGACATGGAGAAGACACTAGAGATGACTGATATTAATTGGAGAGACGGA
CACGATCTGCGTCAAGATATAATTGATTATGAGATAGTACAGCACATATTTGAGATCTTC
CGTTTGACTGTCCAAATGCGTAATTCCCTTTCTGAGCTGGAAGATAGGGACTATGATAGA
TTAATATCCCCTGTACTAAATGAGAACAACATTTTCTATGATAGTGCAAAAGCCGGGGAT
GCATTGCCGAAAGACGCTGACGCTAATGGGGCGTACTGTATAGCTTTAAAGGGGCTTTAC
GAAATAAAGCAGATAACCGAAAACTGGAAGGAAGATGGCAAATTCTCAAGGGACAAACTT
AAGATCTCTAACAAGGATTGGTTCGATTTTATACAAAACAAACGTTATTTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 78
AGCCCAGCGGCTAAAAAAAAGAAACTGGATGGCAGCGTGGATATGAATAATGGTACAAAC
AACTTTCAGAATTTCATTGGGATCTCTAGCTTACAGAAGACCCTGAGGAATGCGTTGATT
CCAACTGAAACAACCCAGCAATTCATCGTGAAAAATGGGATAATCAAAGAGGATGAGTTA
AGGGGTGAAAACCGTCAAATATTGAAGGATATTATGGACGACTACTACCGTGGATTCATC
TCAGAGACGTTGAGCAGCATTGACGACATAGACTGGACTAGCCTTTTCGAGAAGATGGAA
ATTCAGTTAAAGAACGGAGATAACAAAGATACACTAATCAAGGAACAGACAGAATACAGA
AAAGCAATTCATAAGAAATTCGCTAATGACGATCGTTTTAAAAACATGTTCTCTGCAAAA
TTAATTAGCGACATTCTGCCGGAATTCGTTATACATAATAATAACTACAGTGCTTCTGAA
AAGGAAGAGAAAACTCAGGTAATAAAACTGTTCTCTCGTTTTGCCACATCCTTCAAAGAC
TACTTTAAAAATAGAGCGAACTGCTTTAGCGCCGACGATATTAGTTCTTCCTCATGCCAC
AGGATTGTCAACGATAATGCAGAGATATTCTTTTCTAACGCACTAGTCTACAGAAGGATT
GTAAAGTCTTTGTCAAATGATGACATAAACAAGATTAGTGGAGATATGAAAGACTCTCTA
AAGGAAATGAGCCTTGAGGAGATATACTCTTATGAAAAGTACGGTGAGTTTATTACCCAA
GAAGGCATTAGTTTCTATAATGACATTTGTGGAAAAGTTAACAGTTTTATGAATCTATAC
TGTCAAAAAAATAAGGAGAATAAAAATCTTTATAAGTTGCAAAAACTGCATAAGCAGATA
TTATGTATAGCAGACACGAGCTATGAGGTACCGTACAAGTTCGAGAGCGATGAGGAAGTC
TACCAATCTGTCAACGGATTTTTGGACAACATTTCTTCAAAACATATTGTGGAGAGGCTT
AGGAAAATAGGCGACAATTATAATGGATATAACTTAGATAAGATATATATTGTTTCCAAA
TTCTACGAATCTGTAAGCCAGAAGACATACAGAGATTGGGAAACGATAAACACAGCCCTT
GAAATTCACTATAACAACATACTACCTGGAAACGGCAAATCAAAGGCCGACAAAGTTAAG
AAGGCCGTAAAGAATGATTTACAGAAGAGCATAACGGAGATCAATGAGCTGGTGTCTAAC
TATAAATTGTGTAGCGATGACAACATAAAAGCCGAGACTTACATTCACGAAATTTCACAC
ATACTTAACAACTTTGAAGCTCAGGAATTAAAGTATAATCCCGAAATACACCTTGTGGAG
TCCGAACTAAAGGCTAGTGAGCTTAAGAACGTCCTAGACGTAATTATGAATGCCTTCCAC
TGGTGTAGTGTTTTTATGACCGAGGAACTTGTTGACAAAGATAATAATTTTTATGCAGAA
CTAGAAGAGATATACGATGAAATATACCCGGTGATCAGTTTGTACAATCTTGTCAGGAAC
TATGTGACACAAAAGCCCTATTCAACAAAGAAAATAAAACTTAATTTCGGAATTCCTACG
TTAGCTGATGGCTGGTCTAAATCCAAGGAATACAGCAACAACGCTATAATTCTGATGAGA
GATAACTTGTACTATCTAGGCATCTTCAATGCCAAAAATAAGCCTGATAAGAAGATTATA
GAGGGCAACACTTCAGAGAACAAGGGCGACTACAAGAAAATGATCTATAACCTATTGCCT
GGCCCAAACAAGATGATTCCGAAGGTCTTCCTATCATCCAAGACCGGCGTTGAGACATAC
AAGCCATCAGCGTATATTTTAGAGGGGTACAAACAAAACAAGCACATAAAGTCTAGTAAA
GACTTCGATATAACATTTTGTCATGACTTAATTGACTACTTTAAGAATTGCATCGCTATA
CACCCGGAATGGAAGAATTTCGGCTTCGACTTCTCTGATACATCTACCTACGAGGACATT
AGCGGGTTTTACCGTGAAGTCGAATTACAAGGGTATAAGATAGATTGGACGTACATCTCT
GAGAAAGACATAGACTTGCTTCAGGAAAAGGGCCAGTTGTATCTATTCCAAATATACAAT
AAGGATTTTTCCAAGAAATCTACGGGTAATGACAATCTTCACACAATGTATCTTAAGAAC
CTTTTCTCAGAAGAGAACCTGAAGGACATTGTCTTAAAACTAAATGGCGAAGCTGAGATT
TTTTTCAGGAAGTCTTCAATTAAGAACCCGATAATCCACAAGAAGGGGAGTATTCTTGTG
AATAGAACTTACGAGGCCGAAGAAAAAGACCAATTTGGTAACATCCAGATAGTCAGAAAG
AACATTCCAGAGAACATCTACCAAGAGCTATACAAATATTTCAACGACAAGTCCGATAAG
GAACTGTCCGATGAGGCAGCCAAGTTGAAGAATGTCGTGGGTCATCATGAAGCTGCTACT
AACATTGTCAAGGACTATCGTTATACTTACGACAAGTATTTCCTACACATGCCGATAACA
ATTAATTTCAAGGCTAACAAAACAGGCTTTATCAACGATCGTATCTTGCAGTACATAGCT
AAGGAAAAGGATTTGCATGTGATTGGCATTGATAGAGGGGAGCGTAACTTGATATATGTG
TCTGTCATAGACACGTGTGGCAACATCGTCGAACAGAAATCATTCAACATAGTAAACGGC
TACGATTACCAAATTAAGCTGAAACAGCAAGAGGGTGCACGTCAAATTGCGCGTAAAGAG
TGGAAAGAAATTGGTAAAATCAAGGAAATTAAAGAAGGCTACTTGTCTCTTGTTATACAT
GAAATTTCCAAGATGGTTATAAAGTATAACGCGATAATTGCTATGGAAGACTTATCATAC
GGGTTTAAAAAGGGGAGGTTCAAGGTAGAGAGGCAGGTCTATCAAAAGTTCGAGACGATG
TTGATTAATAAACTAAACTATCTAGTGTTCAAAGATATCAGCATTACGGAGAACGGGGGG
CTACTGAAAGGATATCAACTAACGTACATTCCCGATAAGTTAAAGAACGTTGGTCATCAA
TGTGGTTGCATCTTCTACGTGCCTGCTGCCTATACGTCCAAAATAGATCCAACTACTGGA
TTTGTTAACATCTTTAAATTCAAAGATTTAACCGTAGACGCCAAAAGGGAATTTATAAAA
AAATTTGACAGCATCCGTTACGATAGCGAAAAGAATCTGTTCTGTTTTACTTTCGACTAC
AATAATTTCATCACGCAAAATACGGTAATGTCTAAGTCAAGTTGGAGCGTCTACACGTAT
GGAGTCAGGATCAAGAGGCGTTTCGTAAATGGAAGATTCTCTAATGAGTCAGATACTATA
GACATCACGAAAGATATGGAGAAAACCTTGGAGATGACGGATATTAACTGGCGTGATGGA
CACGATTTAAGACAGGACATTATTGACTATGAGATTGTGCAACACATCTTCGAAATATTC
CGTCTAACAGTCCAAATGAGGAATAGCCTAAGTGAATTGGAGGACCGTGATTACGATAGG
CTTATAAGTCCTGTCCTTAACGAAAACAATATTTTCTATGATAGTGCTAAGGCGGGGGAC
GCACTGCCTAAAGACGCAGATGCTAACGGGGCATACTGCATTGCGTTAAAGGGTCTGTAC
GAAATCAAGCAGATTACGGAAAACTGGAAAGAGGATGGCAAGTTTAGCAGAGATAAGTTG
AAGATAAGTAACAAAGATTGGTTTGACTTTATTCAGAATAAAAGGTATTTAAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 79
AGCCCAGCGGCTAAAAAAAAGAAACTGGATGGCAGCGTGGATATGAATAACGGCACTAAT
AATTTCCAGAATTTCATCGGCATTAGCAGCTTACAAAAGACGTTGAGGAATGCCTTAATA
CCCACAGAAACTACTCAACAATTTATAGTGAAGAATGGGATAATTAAGGAAGACGAGTTG
AGAGGTGAAAATAGGCAAATCTTGAAAGACATTATGGATGACTACTACAGGGGCTTCATT
AGTGAAACGTTGTCTTCAATAGATGACATTGATTGGACTTCTTTGTTTGAGAAGATGGAA
ATACAGTTAAAGAACGGCGACAATAAGGATACACTTATCAAAGAGCAAACAGAATATAGA
AAAGCAATTCACAAAAAGTTTGCTAACGATGATAGGTTCAAGAACATGTTTAGCGCTAAA
CTAATATCAGACATCCTTCCCGAGTTCGTTATTCATAACAATAACTATAGTGCAAGTGAA
AAAGAGGAGAAGACACAGGTGATTAAGCTGTTCTCCAGATTCGCGACTTCTTTCAAAGAT
TACTTCAAAAACAGAGCCAACTGTTTTTCAGCTGACGATATCTCTAGTAGTAGTTGTCAC
CGTATAGTGAACGATAACGCTGAGATCTTCTTTAGCAATGCATTAGTGTATAGAAGGATA
GTTAAGTCTCTAAGCAATGATGATATCAATAAAATTTCCGGAGACATGAAGGACTCCCTA
AAGGAAATGTCCTTAGAAGAGATCTACTCATATGAGAAATACGGGGAATTTATTACGCAG
GAAGGGATCTCCTTTTACAATGACATATGCGGGAAGGTCAACTCTTTCATGAACTTATAC
TGCCAAAAGAACAAGGAGAACAAGAATTTATATAAACTTCAGAAACTTCACAAACAAATA
CTGTGCATAGCCGATACCTCATATGAGGTTCCTTACAAATTTGAATCAGATGAAGAGGTA
TACCAATCCGTTAACGGCTTTCTTGACAATATTAGCTCAAAGCACATCGTGGAGAGGTTG
AGAAAGATTGGTGATAATTATAATGGCTACAATCTAGATAAGATATATATTGTTAGCAAG
TTCTACGAGTCTGTGTCCCAAAAAACATATAGGGATTGGGAGACAATTAATACTGCTCTA
GAAATCCATTACAACAACATCCTTCCTGGAAATGGCAAGAGTAAGGCCGACAAAGTCAAG
AAAGCAGTGAAAAATGATCTGCAAAAATCAATTACTGAGATAAACGAGCTAGTATCTAAT
TACAAGCTTTGTAGCGACGATAACATTAAGGCAGAAACGTACATACACGAGATTAGTCAC
ATCTTAAATAATTTTGAAGCCCAAGAACTGAAATATAACCCTGAGATACACCTTGTTGAA
TCCGAGTTAAAGGCGTCTGAACTAAAAAACGTGTTAGACGTTATTATGAATGCCTTCCAC
TGGTGTAGCGTCTTTATGACTGAGGAGTTGGTTGATAAGGATAATAACTTTTACGCTGAA
TTGGAAGAAATTTATGACGAAATCTATCCTGTTATTTCTCTATATAATTTGGTGAGAAAT
TACGTAACGCAAAAGCCCTATAGTACGAAAAAAATAAAACTAAATTTCGGGATCCCTACC
CTAGCCGACGGTTGGTCTAAATCCAAGGAGTACTCAAACAATGCAATAATATTGATGAGG
GACAACCTGTACTACCTAGGCATATTTAATGCCAAAAATAAGCCCGATAAAAAGATTATA
GAAGGGAACACGTCAGAAAATAAAGGAGACTATAAGAAAATGATCTACAACCTTTTGCCC
GGCCCCAATAAAATGATCCCGAAGGTCTTCCTAAGTAGCAAGACTGGCGTAGAGACCTAC
AAACCATCTGCATACATTTTGGAGGGGTACAAGCAAAACAAGCACATAAAGAGTAGTAAG
GATTTTGACATTACATTCTGCCATGACTTAATTGACTACTTTAAAAATTGCATCGCAATT
CACCCTGAATGGAAAAATTTTGGATTTGATTTCTCTGATACTTCAACATATGAGGATATT
TCAGGGTTCTACAGGGAGGTCGAACTACAGGGTTACAAAATAGACTGGACGTATATTTCT
GAGAAAGATATAGATTTGCTTCAGGAAAAGGGTCAGCTATATCTGTTCCAGATATATAAT
AAGGACTTCTCCAAAAAGAGTACCGGAAATGATAATCTGCACACAATGTACTTAAAAAAC
TTGTTCTCTGAGGAGAATCTAAAAGACATCGTACTAAAACTTAACGGGGAGGCCGAAATT
TTTTTTAGGAAGTCCAGCATCAAGAACCCGATTATTCATAAAAAAGGTAGCATTTTGGTG
AACCGTACTTATGAGGCGGAAGAAAAAGACCAATTCGGTAATATTCAAATCGTTAGAAAG
AACATCCCTGAGAACATTTATCAGGAACTATACAAATACTTTAACGACAAATCAGATAAG
GAGCTTTCTGATGAGGCAGCTAAATTGAAAAATGTAGTGGGACATCACGAAGCAGCCACT
AACATAGTGAAGGACTACAGATACACATACGATAAGTACTTCCTGCACATGCCTATTACA
ATTAACTTTAAAGCAAATAAAACAGGGTTTATTAACGACAGAATCTTACAGTATATTGCC
AAAGAAAAGGATCTGCATGTGATAGGAATAGACAGAGGAGAAAGAAACCTGATATACGTC
TCCGTGATTGATACATGTGGGAACATAGTAGAACAGAAGTCCTTTAACATTGTTAATGGG
TACGATTATCAAATTAAATTAAAACAACAAGAAGGAGCACGTCAAATAGCTAGGAAAGAA
TGGAAAGAGATAGGAAAAATTAAGGAAATTAAGGAGGGTTACCTGTCCCTTGTAATTCAT
GAAATATCCAAAATGGTAATTAAATATAACGCGATCATCGCGATGGAAGATCTAAGCTAC
GGGTTCAAAAAAGGCAGGTTTAAGGTGGAGAGGCAAGTTTACCAAAAGTTCGAGACAATG
TTGATTAATAAGTTAAACTACTTAGTTTTCAAAGATATCTCCATAACCGAGAATGGCGGG
CTTTTAAAAGGGTACCAACTAACATATATCCCGGATAAATTGAAGAACGTTGGACACCAG
TGTGGCTGCATATTTTATGTACCCGCTGCGTATACTTCTAAAATTGACCCGACCACCGGG
TTTGTAAACATATTCAAGTTTAAGGACCTAACAGTTGACGCCAAACGTGAGTTCATCAAG
AAGTTCGATAGTATAAGGTATGACTCTGAGAAGAACCTTTTCTGCTTCACGTTTGACTAT
AATAATTTCATCACCCAAAATACAGTTATGTCAAAAAGCTCTTGGTCAGTATATACGTAT
GGCGTAAGGATTAAGCGTAGGTTCGTGAACGGTAGATTTTCCAACGAGTCAGATACTATT
GATATTACCAAGGATATGGAGAAGACATTAGAAATGACAGATATAAATTGGAGGGATGGG
CACGATCTAAGGCAAGATATCATTGATTACGAAATTGTTCAGCACATATTCGAGATATTC
CGTCTTACAGTACAAATGCGTAACAGCTTGTCTGAGTTGGAAGATCGTGACTATGACAGG
TTGATATCACCGGTCTTGAACGAGAACAATATATTCTACGACAGCGCTAAGGCGGGAGAC
GCTCTGCCTAAAGACGCAGATGCCAATGGGGCGTACTGCATTGCCTTAAAAGGCTTATAC
GAGATTAAACAGATCACAGAGAACTGGAAAGAGGACGGCAAGTTTTCTAGAGATAAATTG
AAAATCTCAAACAAAGACTGGTTCGATTTCATCCAAAACAAAAGATACCTTAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 80
AGCCCAGCGGCTAAAAAAAAGAAACTGGATGGCAGCGTGGATATGAACAATGGAACTAAC
AACTTCCAGAACTTTATCGGCATCTCTTCCCTCCAAAAGACACTGAGAAATGCACTGATC
CCAACCGAAACGACTCAACAATTTATTGTTAAGAACGGCATCATAAAAGAAGACGAGCTT
CGCGGCGAGAACCGCCAGATACTTAAGGATATTATGGACGATTATTACCGAGGCTTTATC
AGCGAAACTCTTAGCTCTATTGATGATATCGACTGGACCTCCCTCTTCGAAAAAATGGAG
ATACAGCTCAAGAACGGCGATAATAAAGACACCTTGATAAAGGAACAGACTGAGTACAGG
AAAGCGATCCACAAGAAATTCGCGAACGACGACAGGTTTAAAAACATGTTCTCTGCAAAA
TTGATATCCGACATCTTGCCGGAATTTGTGATACACAACAATAACTATAGCGCTTCAGAG
AAAGAAGAGAAGACCCAAGTAATCAAGTTGTTCAGCCGCTTCGCAACGTCTTTTAAAGAT
TACTTTAAGAACCGGGCCAATTGTTTCTCCGCGGATGATATTAGCTCATCAAGTTGCCAT
CGAATTGTCAATGATAATGCGGAGATCTTCTTCAGCAATGCGCTGGTCTACAGACGAATC
GTAAAAAGTCTTTCAAATGACGACATCAATAAGATTAGTGGAGATATGAAGGATTCCCTT
AAGGAAATGAGTCTTGAAGAAATATACTCATACGAAAAGTACGGGGAATTTATTACCCAG
GAGGGGATCTCCTTCTATAACGACATCTGTGGAAAAGTAAACTCATTCATGAACCTGTAC
TGTCAGAAAAACAAAGAAAACAAAAATCTGTATAAACTCCAAAAATTGCACAAGCAAATA
TTGTGTATAGCGGACACATCATACGAGGTTCCATATAAGTTCGAAAGTGATGAAGAAGTC
TACCAATCAGTGAATGGGTTTCTGGACAACATTAGTTCCAAGCACATAGTTGAACGACTG
CGAAAGATTGGTGACAATTACAACGGCTATAATTTGGACAAGATTTATATAGTTAGCAAA
TTTTATGAATCCGTATCACAAAAGACTTATAGAGACTGGGAAACAATCAACACGGCACTT
GAGATCCATTATAACAATATTCTTCCAGGGAACGGCAAAAGCAAGGCTGATAAGGTAAAA
AAGGCCGTTAAGAATGATCTTCAAAAATCCATAACGGAGATCAACGAACTTGTAAGTAAC
TACAAATTGTGCTCTGACGACAATATAAAGGCTGAAACGTATATTCACGAGATTAGCCAT
ATCCTGAATAACTTTGAGGCCCAAGAACTCAAGTATAACCCGGAAATACATTTGGTAGAA
AGCGAGCTTAAAGCGAGTGAGCTGAAAAACGTCCTCGATGTGATCATGAATGCTTTCCAC
TGGTGTAGTGTCTTTATGACTGAGGAGTTGGTTGATAAAGACAATAATTTCTACGCTGAA
CTGGAAGAAATTTACGACGAAATCTATCCAGTGATCTCCCTCTATAACCTCGTTCGAAAC
TACGTGACGCAGAAACCTTATTCTACAAAGAAAATTAAGTTGAACTTCGGCATTCCTACA
CTTGCTGACGGATGGTCCAAATCCAAAGAGTACTCAAACAACGCAATCATCCTCATGCGG
GATAACCTTTATTATTTGGGCATTTTCAACGCCAAAAACAAACCTGATAAAAAGATAATT
GAAGGCAATACGAGTGAGAACAAGGGCGACTACAAAAAAATGATATATAACTTGTTGCCA
GGCCCCAACAAGATGATTCCTAAAGTTTTTCTGTCTTCTAAGACTGGAGTTGAAACTTAC
AAACCCTCCGCCTACATTCTTGAAGGGTATAAACAGAATAAGCACATAAAGTCCTCAAAG
GATTTCGACATTACGTTTTGCCATGACCTCATCGACTATTICAAGAACTGTATCGCCATA
CATCCGGAGTGGAAGAATTTTGGATTTGATTTCTCCGACACATCTACCTATGAAGACATA
AGCGGTTTCTACCGGGAGGTCGAGCTTCAGGGCTATAAGATAGATTGGACATACATTAGT
GAAAAAGATATCGATCTTCTGCAAGAAAAGGGACAACTTTACCTTTTTCAGATTTATAAT
AAAGACTTTTCAAAAAAGTCCACAGGGAACGATAATCTGCACACCATGTATCTCAAGAAT
CTGTTTAGTGAAGAAAACCTTAAAGACATAGTTTTGAAGCTTAACGGAGAGGCTGAGATT
TTTTTTAGAAAGTCCTCAATTAAAAACCCTATAATACACAAGAAAGGCTCTATTCTTGTT
AACAGGACATATGAAGCCGAGGAGAAAGATCAGTTTGGCAATATCCAGATTGTTCGCAAG
AATATCCCGGAAAATATATATCAGGAGCTGTATAAATACTTTAACGACAAGAGCGACAAG
GAGCTGAGTGACGAGGCCGCGAAGCTTAAGAATGTAGTAGGTCACCACGAAGCAGCCACC
AATATCGTCAAAGACTATAGGTACACGTACGACAAGTACTTTTTGCACATGCCTATAACT
ATAAACTTCAAAGCTAATAAAACTGGGTTTATTAATGACAGGATTCTCCAATACATCGCT
AAAGAGAAGGATCTGCATGTAATTGGCATAGACAGAGGTGAGAGAAACTTGATATATGTC
AGCGTAATAGACACATGTGGCAATATCGTGGAACAGAAGTCTTTTAACATCGTCAATGGT
TACGACTACCAAATTAAGTTGAAACAGCAGGAAGGCGCACGACAGATCGCACGAAAGGAA
TGGAAAGAGATAGGCAAAATAAAAGAAATAAAGGAGGGCTATCTCAGTCTCGTTATACAC
GAAATTTCAAAAATGGTTATTAAGTACAATGCAATCATAGCGATGGAGGATCTCAGTTAT
GGGTTCAAAAAGGGTCGGTTTAAAGTTGAGCGCCAAGTGTACCAAAAGTTCGAGACAATG
CTGATTAACAAGCTGAACTACCTCGTCTTCAAAGATATAAGTATTACGGAGAACGGTGGC
CTTCTTAAAGGCTATCAACTTACTTACATCCCGGACAAGCTCAAAAACGTAGGGCACCAA
TGCGGGTGTATTTTCTATGTGCCTGCGGCATATACGTCAAAGATTGACCCAACCACAGGA
TTCGTAAACATATTCAAGTTTAAGGACCTCACCGTTGATGCGAAAAGGGAGTTCATTAAA
AAATTTGATTCTATTCGATATGATAGTGAGAAAAATCTCTTTTGTTTCACATTTGACTAT
AATAATTTTATTACTCAGAATACTGTCATGAGCAAGTCATCTTGGTCAGTGTACACATAC
GGGGTGCGGATCAAACGCAGGTTCGTCAATGGTCGCTTCTCAAACGAATCAGACACCATT
GACATCACAAAGGACATGGAAAAAACCCTTGAGATGACCGACATTAATTGGCGCGATGGT
CATGATCTGCGGCAAGACATCATAGACTACGAAATCGTCCAACACATCTTTGAGATCTTT
CGCTTGACGGTCCAAATGCGGAACTCCCTGTCCGAGCTCGAGGATAGAGATTATGATCGG
CTGATATCTCCCGTGCTTAATGAAAATAACATCTTCTACGACTCCGCCAAGGCGGGTGAT
GCCCTGCCGAAGGATGCGGATGCTAATGGCGCTTATTGCATTGCTCTTAAGGGGCTCTAT
GAGATAAAGCAGATCACGGAAAACTGGAAAGAAGACGGTAAGTTTAGTAGAGACAAGCTG
AAGATCTCAAATAAAGACTGGTTTGATTTCATACAGAACAAGCGGTACCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 81
AGCCCAGCGGCTAAAAAAAAGAAACTGGATGGCAGCGTGGATATGAACAATGGCACTAAC
AATTTTCAGAATTTCATCGGCATTTCAAGTCTGCAAAAAACTCTGAGGAATGCTTTGATC
CCTACTGAAACCACTCAGCAATTTATAGTCAAGAACGGTATAATTAAAGAAGATGAACTC
AGGGGTGAAAATAGACAAATACTCAAGGACATTATGGATGACTATTATAGAGGCTTCATC
TCAGAGACTCTCTCATCAATAGATGATATCGATTGGACTAGCCTTTTCGAGAAAATGGAG
ATTCAGTTGAAAAATGGTGATAACAAAGATACGTTGATAAAGGAACAGACCGAGTACAGG
AAAGCCATTCATAAGAAATTTGCTAATGACGATAGATTTAAGAATATGTTTAGTGCAAAA
CTGATTAGTGACATTCTGCCGGAGTTCGTTATCCATAATAATAACTACTCTGCATCCGAA
AAGGAGGAAAAGACGCAAGTTATTAAACTGTTCAGCCGCTTCGCCACAAGCTTCAAGGAC
TACTTCAAAAATAGAGCCAACTGCTTTTCTGCCGACGATATATCATCATCTTCATGCCAT
CGGATCGTTAACGATAACGCCGAGATATTCTTCAGCAACGCCCTTGTATATCGAAGAATA
GTCAAAAGTCTGAGTAATGATGATATTAATAAAATTAGCGGTGATATGAAAGACTCCCTG
AAGGAAATGTCACTGGAGGAAATTTATAGTTACGAAAAGTACGGCGAATTCATTACTCAA
GAAGGCATATCCTTCTATAACGACATTTGCGGAAAGGTCAACTCATTCATGAACCTTTAT
TGCCAGAAGAATAAGGAGAATAAAAATCTTTACAAATTGCAAAAACTTCACAAACAAATT
CTTTGCATCGCGGATACGTCCTACGAAGTTCCTTACAAATTTGAATCCGATGAGGAAGTG
TATCAGAGTGTCAATGGATTTTTGGATAATATCTCTTCAAAACATATTGTGGAGAGATTG
CGCAAAATAGGTGATAACTACAATGGCTACAACCTGGACAAGATTTATATTGTTAGCAAG
TTCTATGAAAGTGTCAGTCAAAAGACCTACAGAGATTGGGAGACAATCAACACGGCGCTC
GAAATACACTACAATAACATCCTCCCCGGCAATGGGAAGAGTAAAGCCGATAAGGTTAAA
AAAGCTGTTAAGAACGACCTCCAGAAATCCATCACGGAAATAAACGAGCTGGTTTCCAAC
TATAAGCTGTGTAGCGATGATAATATTAAGGCTGAGACATATATACATGAGATCAGCCAC
ATTCTCAACAATTTCGAGGCACAGGAACTCAAATACAATCCCGAGATTCACTTGGTGGAA
AGTGAGTTGAAGGCGTCAGAGCTTAAGAATGTACTTGACGTAATAATGAATGCTTTTCAT
TGGTGCTCCGTGTTCATGACTGAGGAACTCGTGGATAAGGATAATAACTTTTATGCGGAG
TTGGAAGAGATATACGATGAAATATACCCGGTTATCTCACTGTATAATCTGGTCAGAAAT
TACGTGACCCAAAAGCCTTATAGTACAAAAAAAATAAAGTTGAACTTCGGTATTCCGACA
TTGGCAGATGGTTGGTCCAAAAGCAAAGAATACTCTAATAACGCCATTATATTGATGCGA
GACAATTTGTATTACCTTGGGATCTTTAACGCGAAAAACAAACCGGATAAGAAGATCATC
GAAGGTAATACATCTGAGAATAAGGGGGATTACAAGAAGATGATTTATAATCTGTTGCCG
GGGCCAAACAAGATGATTCCGAAGGTCTTTCTGTCATCTAAGACAGGAGTAGAGACCTAC
AAACCTTCTGCGTACATTTTGGAAGGCTACAAACAGAACAAGCATATAAAATCTAGCAAG
GACTTTGATATCACGTTTTGTCATGATCTGATAGATTATTTCAAAAACTGCATCGCTATA
CATCCTGAGTGGAAGAATTTCGGCTTTGACTTTTCTGACACCAGCACATACGAAGACATC
TCAGGTTTCTACCGGGAAGTCGAGCTCCAGGGGTACAAGATTGACTGGACATATATAAGT
GAAAAAGACATCGACCTCCTCCAAGAGAAGGGCCAACTTTACCTGTTCCAGATCTATAAC
AAAGACTTTTCTAAAAAGTCCACGGGTAACGACAACTTGCACACTATGTATCTGAAAAAC
TTGTTCTCTGAAGAGAACCTCAAGGACATCGTCCTGAAGCTTAACGGGGAGGCGGAGATC
TTCTTTAGAAAGTCCTCTATCAAAAATCCCATTATCCATAAAAAGGGCTCTATACTCGTT
AATAGGACATATGAAGCGGAGGAAAAAGATCAATTTGGGAACATCCAGATCGTCCGGAAA
AATATACCTGAGAATATCTATCAAGAGCTGTACAAGTATTTTAATGATAAGTCAGACAAA
GAGCTCAGTGATGAGGCGGCAAAGCTCAAGAACGTGGTGGGGCATCATGAAGCTGCGACG
AACATTGTCAAAGATTATAGATACACTTACGATAAATACTTCCTCCACATGCCGATAACG
ATTAACTTCAAAGCCAATAAGACGGGGTTTATAAATGATCGGATCCTTCAGTACATTGCG
AAAGAGAAAGACCTCCATGTGATCGGAATTGACCGAGGAGAAAGGAATCTGATTTACGTG
TCCGTGATTGATACTTGCGGGAATATAGTCGAGCAAAAGAGTTTCAACATAGTCAACGGG
TATGACTATCAGATAAAGCTCAAACAGCAGGAAGGTGCGAGGCAAATTGCGCGCAAAGAG
TGGAAGGAGATAGGCAAGATTAAAGAAATCAAGGAAGGTTATCTCAGCTTGGTGATCCAT
GAAATATCTAAGATGGTTATAAAGTACAATGCCATAATAGCCATGGAGGATCTTTCCTAC
GGGTTTAAGAAGGGCCGATTTAAAGTGGAGCGACAAGTTTACCAGAAGTTCGAAACCATG
TTGATTAACAAACTTAACTATTTGGTGTTCAAGGATATAAGTATAACCGAAAACGGCGGT
TTGCTTAAGGGTTATCAGCTCACGTATATTCCTGATAAACTTAAAAACGTTGGACACCAG
TGTGGATGTATCTTCTACGTGCCAGCCGCTTACACTAGTAAGATAGATCCTACCACGGGG
TTTGTGAATATTTTTAAGTTTAAAGACTTGACAGTCGACGCCAAAAGGGAATTTATAAAA
AAGTTTGATTCTATCCGCTACGATAGTGAAAAAAATCTCTTTTGCTTTACTTTCGACTAT
AACAACTTCATTACGCAGAACACTGTCATGAGTAAGTCCAGCTGGAGCGTCTACACATAT
GGCGTCCGAATTAAACGACGATTTGTAAACGGGCGGTTTTCAAACGAATCTGACACGATA
GACATTACCAAGGATATGGAGAAGACACTTGAGATGACCGACATAAACTGGCGGGACGGT
CACGATCTTCGGCAGGACATAATTGATTACGAAATCGTCCAGCATATATTCGAAATATTT
CGACTTACAGTGCAAATGCGGAACAGTCTCTCTGAACTGGAAGATCGCGATTATGACCGG
TTGATTTCTCCGGTCCTCAATGAAAATAACATATTTTATGATAGTGCTAAGGCAGGTGAT
GCGTTGCCAAAGGATGCAGACGCTAATGGTGCCTATTGTATCGCGCTCAAGGGATTGTAC
GAGATAAAGCAAATTACGGAGAACTGGAAGGAGGATGGTAAGTTTAGCCGAGACAAGTTG
AAGATTAGCAATAAAGACTGGTTTGATTTTATCCAAAACAAGAGGTACCTGAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 82
AGCCCAGCGGCTAAAAAAAAGAAACTGGATGGCAGCGTGGATATGAATAACGGAACTAAT
AACTTTCAAAATTTCATAGGTATTTCAAGCTTGCAGAAGACCCTGAGGAATGCCCTGATT
CCAACCGAGACAACGCAGCAGTTCATAGTCAAAAATGGCATTATTAAGGAAGATGAGCTG
CGGGGGGAAAACCGACAGATACTCAAGGATATTATGGACGACTATTACCGGGGATTTATC
TCAGAAACGCTGAGCAGTATTGATGACATCGATTGGACCAGTCTTTTCGAGAAAATGGAA
ATTCAACTTAAGAATGGTGACAATAAAGACACTCTCATAAAGGAGCAAACTGAATACCGA
AAAGCCATACACAAAAAGTTTGCCAACGATGACCGCTTTAAAAACATGTTTTCAGCTAAG
CTCATTAGCGACATTCTCCCCGAGTTTGTGATTCATAACAATAACTATAGCGCATCCGAG
AAGGAGGAAAAAACCCAAGTTATCAAATTGTTCAGTAGATTCGCTACGAGCTTTAAAGAT
TACTTTAAAAACCGGGCTAACTGCTTCAGTGCAGACGATATCAGCTCCTCATCCTGTCAT
CGCATCGTCAATGATAATGCTGAGATCTTCTTTTCTAATGCACTGGTTTACCGCAGGATA
GTTAAGTCTCTTAGTAACGACGACATCAACAAGATATCAGGAGATATGAAGGATTCCCTT
AAAGAAATGAGTCTCGAGGAGATATATTCTTATGAAAAATACGGCGAATTTATTACCCAA
GAGGGCATTAGTTTCTATAATGACATATGCGGAAAAGTTAATAGTTTTATGAATCTCTAT
TGTCAGAAGAATAAGGAGAATAAGAACCTCTACAAATTGCAGAAGTTGCACAAGCAAATT
CTGTGTATCGCGGACACCTCTTACGAGGTCCCATATAAGTTCGAGAGTGATGAAGAAGTA
TACCAGAGCGTTAATGGGTTCCTGGACAACATCTCAAGTAAACACATAGTCGAAAGGCTC
CGAAAGATCGGTGATAACTATAACGGATATAATTTGGATAAAATTTATATAGTTAGCAAA
TTTTACGAGAGCGTCAGTCAGAAGACCTACCGGGACTGGGAGACCATAAACACAGCGCTG
GAAATACATTATAACAACATACTGCCTGGGAACGGTAAGTCAAAGGCAGACAAGGTTAAA
AAGGCTGTGAAGAATGACCTGCAAAAATCAATTACAGAAATAAATGAGTTGGTAAGTAAT
TACAAACTTTGCAGCGATGATAATATAAAGGCAGAGACGTACATACATGAAATATCTCAT
ATCCTCAACAATTTCGAAGCCCAAGAACTGAAGTACAACCCGGAAATTCATCTTGTAGAG
TCTGAGTTGAAGGCCTCCGAATTGAAAAACGTTCTTGACGTAATTATGAATGCCTTCCAC
TGGTGCTCAGTATTCATGACGGAAGAGCTCGTGGATAAAGACAACAATTTTTACGCTGAA
CTGGAAGAAATATATGACGAGATTTACCCCGTAATTTCACTCTACAACTTGGTACGAAAT
TACGTTACCCAAAAGCCATACTCAACAAAAAAAATTAAACTGAACTTCGGGATACCCACC
CTCGCAGATGGATGGTCAAAGTCCAAAGAGTACAGTAACAATGCAATTATCCTGATGCGA
GACAACCTTTATTACCTCGGGATTTTCAACGCTAAAAATAAACCTGATAAAAAAATAATT
GAGGGTAATACCTCTGAAAACAAGGGGGATTATAAAAAGATGATATACAATCTGCTGCCT
GGCCCGAACAAAATGATTCCTAAAGTCTTCTTGTCTTCCAAGACTGGAGTCGAAACCTAC
AAGCCAAGTGCTTATATACTCGAAGGGTACAAACAAAATAAGCACATAAAATCCAGCAAG
GATTTTGATATTACATTCTGCCACGATTTGATTGATTATTTTAAGAACTGTATAGCCATC
CACCCAGAATGGAAGAATTTTGGTTTTGATTTTAGCGATACCTCAACATATGAGGATATC
TCTGGCTTTTACCGCGAGGTAGAACTGCAAGGTTATAAGATCGATTGGACTTATATTTCT
GAAAAGGACATAGATCTCCTGCAAGAGAAAGGGCAACTTTATTTGTTTCAAATATACAAC
AAAGATTTTAGTAAGAAGAGTACTGGCAATGATAACCTTCACACTATGTATCTGAAGAAC
CTTTTTTCTGAGGAGAACTTGAAGGACATAGTCCTTAAACTCAATGGGGAAGCTGAAATA
TTCTTTCGCAAAAGCTCCATTAAAAACCCGATCATTCATAAAAAGGGTTCCATCTTGGTA
AACCGCACATACGAGGCGGAAGAAAAAGATCAGTTCGGAAATATCCAGATCGTAAGGAAG
AATATCCCCGAAAATATATACCAAGAGCTTTACAAATATTTTAACGATAAGTCAGACAAG
GAACTGTCAGACGAAGCAGCCAAGTTGAAGAATGTCGTAGGGCACCACGAAGCAGCTACA
AACATAGTTAAAGATTATCGGTACACCTACGATAAATATTTCCTGCATATGCCAATAACC
ATAAACTTCAAAGCCAACAAAACAGGGTTCATCAATGACCGAATACTTCAGTATATAGCC
AAGGAAAAAGACCTGCATGTTATAGGAATAGATAGAGGTGAGCGCAACTTGATATATGTC
AGCGTGATAGACACCTGCGGAAATATCGTCGAGCAAAAAAGTTTCAACATTGTTAATGGC
TACGATTACCAAATTAAATTGAAGCAGCAAGAGGGGGCTCGGCAAATCGCGCGAAAGGAA
TGGAAAGAAATCGGGAAGATTAAAGAAATTAAAGAGGGCTACCTGTCTCTTGTAATTCAC
GAAATATCTAAGATGGTCATCAAGTATAATGCCATTATTGCGATGGAAGATCTGTCCTAC
GGATTTAAGAAAGGCAGGTTTAAAGTCGAAAGGCAGGTGTACCAGAAATTCGAGACCATG
CTGATTAATAAGCTCAACTATCTCGTATTTAAGGATATTTCTATAACTGAAAATGGAGGG
CTTCTCAAAGGATATCAACTCACATACATACCTGATAAGCTGAAGAACGTAGGCCACCAG
TGTGGATGCATATTCTATGTACCAGCTGCATACACAAGCAAGATCGATCCAACTACTGGG
TTTGTCAATATCTTCAAATTTAAGGACTTGACGGTCGATGCCAAACGGGAGTTCATCAAA
AAGTTTGATAGTATTCGATATGATAGTGAGAAGAACTTGTTTTGCTTCACATTTGACTAC
AACAATTTCATAACGCAAAATACGGTTATGTCTAAATCCTCATGGAGCGTCTACACTTAC
GGAGTGAGGATAAAGCGGCGCTTCGTAAATGGCAGGTTTAGCAATGAATCCGACACGATT
GACATAACCAAGGATATGGAGAAAACCCTCGAGATGACCGATATAAATTGGCGGGATGGA
CACGATCTGCGACAAGACATAATCGATTATGAAATCGTGCAGCACATATTTGAGATATTC
AGGCTTACGGTCCAAATGAGAAATTCCCTTTCCGAACTTGAAGACCGCGATTACGACCGA
CTGATAAGCCCCGTTCTGAACGAAAATAACATCTTCTACGACAGCGCTAAAGCGGGAGAC
GCGCTGCCGAAAGATGCGGACGCAAATGGAGCCTATTGTATCGCCTTGAAAGGGTTGTAC
GAGATCAAACAGATAACCGAGAATTGGAAGGAGGATGGGAAGTTTAGTCGAGACAAACTT
AAAATAAGCAACAAGGACTGGTTCGACTTTATTCAAAACAAACGATATCTCAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 83
AGCCCAGCGGCTAAAAAAAAGAAACTGGATGGCAGCGTGGATATGAATAATGGTACTAAC
AATTTTCAAAACTTTATCGGCATCTCTTCACTTCAGAAAACTCTTCGGAACGCCCTTATA
CCGACGGAGACAACGCAGCAGTTTATAGTTAAAAACGGGATCATTAAAGAAGATGAACTC
AGAGGGGAAAACAGGCAAATATTGAAGGACATTATGGACGATTACTACCGGGGGTTTATT
TCAGAGACCCTTTCATCTATTGATGACATAGATTGGACCTCCCTTTTCGAGAAAATGGAG
ATACAATTGAAAAACGGCGACAATAAAGATACACTTATCAAGGAACAAACTGAGTATCGC
AAGGCGATTCACAAGAAGTTTGCGAATGACGATCGCTTTAAGAATATGTTTTCTGCGAAG
CTCATAAGTGACATTCTGCCTGAATTTGTCATTCATAACAACAATTATTCTGCTAGCGAA
AAAGAGGAAAAAACTCAAGTCATTAAGCTTTTTAGCAGGTTCGCTACTAGTTTTAAAGAC
TATTTTAAGAACCGGGCGAATTGCTTTAGCGCTGACGACATATCATCCTCATCCTGTCAT
CGCATAGTCAATGATAATGCAGAAATATTCTTTTCTAATGCGCTCGTGTATCGGAGAATA
GTGAAAAGCCTCTCTAACGATGACATTAACAAAATAAGCGGCGATATGAAGGATAGTCTG
AAGGAAATGTCCCTCGAAGAAATATACTCATACGAGAAGTACGGAGAATTTATCACCCAG
GAAGGAATTAGTTTTTACAACGACATCTGTGGTAAGGTTAACTCTTTTATGAATCTGTAT
TGTCAAAAGAATAAAGAAAATAAAAATCTTTATAAGCTCCAAAAGCTTCACAAACAAATC
TTGTGCATTGCGGATACGTCATACGAAGTACCTTACAAATTTGAAAGCGACGAAGAGGTG
TATCAGTCAGTGAATGGGTTCCTTGACAATATTTCTAGCAAACATATTGTGGAGCGACTT
CGAAAGATCGGTGATAATTACAATGGCTATAATTTGGATAAAATTTACATAGTTAGTAAG
TTTTATGAATCCGTCTCACAAAAGACGTACCGAGATTGGGAGACCATCAACACTGCTCTG
GAGATTCATTACAATAATATATTGCCTGGGAATGGGAAGTCAAAGGCCGACAAGGTTAAA
AAAGCCGTAAAAAACGATCTTCAAAAGTCCATTACCGAGATAAATGAACTTGTATCCAAC
TATAAGTTGTGCTCTGACGATAATATTAAAGCAGAAACGTATATCCACGAAATAAGTCAC
ATCCTGAACAACTTCGAAGCTCAAGAGCTCAAGTATAATCCTGAAATTCATCTCGTCGAA
AGCGAGCTGAAAGCATCCGAGTTGAAGAATGTGCTTGATGTGATCATGAACGCATTCCAT
TGGTGCAGTGTGTTCATGACCGAAGAACTTGTAGACAAAGACAACAACTTCTACGCTGAA
TTGGAAGAGATTTACGATGAAATTTACCCCGTGATATCCCTCTATAATCTGGTAAGAAAT
TACGTCACGCAAAAACCATACAGTACCAAGAAAATAAAGCTCAACTTTGGTATTCCGACG
TTGGCAGATGGGTGGAGTAAGAGCAAGGAGTATTCTAACAATGCAATCATCCTCATGCGC
GACAATTTGTATTATCTGGGGATCTTCAACGCGAAAAATAAGCCCGACAAAAAGATAATA
GAAGGCAATACGTCCGAGAACAAAGGGGACTATAAGAAAATGATTTATAACCTTCTTCCA
GGACCCAACAAGATGATCCCAAAGGTTTTCTTGAGTTCAAAAACCGGCGTAGAAACTTAT
AAACCGTCCGCCTACATTCTGGAAGGGTACAAGCAAAACAAGCACATTAAGTCATCTAAG
GATTTCGACATTACTTTTTGTCATGATTTGATAGACTACTTCAAAAATTGTATAGCGATA
CATCCGGAATGGAAAAATTTTGGGTTCGATTTTTCCGACACAAGTACTTATGAAGACATC
TCAGGGTTTTATAGGGAAGTTGAACTGCAAGGTTACAAAATAGACTGGACTTATATTAGT
GAGAAGGACATTGATTTGCTCCAGGAAAAGGGTCAATTGTATCTGTTCCAGATATATAAC
AAGGATTTCTCTAAAAAATCTACAGGTAACGACAATCTCCACACGATGTACCTCAAGAAT
CTCTTCAGCGAAGAGAATTTGAAGGATATCGTACTTAAGCTCAATGGAGAAGCGGAAATA
TTCTTCAGAAAGTCCAGCATTAAGAATCCTATAATTCACAAGAAAGGGTCAATTCTCGTA
AACCGGACTTATGAGGCCGAAGAAAAAGATCAGTTTGGTAACATTCAGATTGTACGGAAA
AACATTCCCGAGAACATCTATCAAGAACTGTATAAATACTTTAATGATAAATCCGACAAG
GAACTTTCTGACGAGGCTGCAAAATTGAAGAACGTAGTGGGACACCATGAGGCCGCAACC
AATATAGTAAAGGATTACAGATACACTTATGATAAGTATTTCCTCCATATGCCGATCACG
ATTAATTTCAAGGCGAATAAAACCGGCTTCATTAACGATCGCATTTTGCAATATATTGCG
AAGGAAAAGGATTTGCACGTGATAGGTATAGACCGGGGTGAACGAAACTTGATTTACGTC
TCTGTGATCGACACATGCGGAAATATAGTTGAACAGAAGTCCTTTAATATTGTGAATGGT
TACGACTACCAGATAAAATTGAAGCAACAGGAGGGCGCAAGACAGATAGCTCGCAAAGAG
TGGAAGGAAATCGGCAAGATCAAAGAAATAAAGGAGGGTTATCTTTCCCTGGTAATTCAT
GAAATTAGCAAGATGGTTATTAAGTATAATGCTATAATAGCTATGGAGGACCTTTCCTAT
GGGTTCAAGAAAGGTCGCTTCAAAGTGGAGCGACAAGTGTATCAAAAGTTCGAGACTATG
TTGATAAATAAATTGAATTATTTGGTTTTTAAAGACATTTCAATAACTGAGAACGGGGGT
CTCTTGAAGGGGTACCAATTGACTTATATTCCGGACAAGTTGAAGAATGTCGGACACCAG
TGTGGTTGCATTTTCTACGTGCCTGCCGCTTACACCTCAAAAATCGATCCGACCACTGGT
TTTGTAAATATATTTAAATTCAAAGATCTCACCGTTGATGCCAAACGGGAGTTTATCAAA
AAATTCGATTCCATTCGCTACGACTCTGAGAAAAACCTTTTTTGTTTCACGTTCGATTAT
AACAACTTTATAACCCAAAATACTGTAATGTCCAAGTCAAGTTGGTCTGTCTATACTTAC
GGAGTAAGGATCAAGCGCCGCTTCGTTAATGGGAGATTCTCAAACGAGTCTGATACCATA
GACATAACTAAAGACATGGAAAAAACCCTGGAAATGACGGACATCAATTGGCGAGACGGG
CATGATCTTCGACAGGACATAATAGATTACGAAATTGTTCAACACATTTTCGAGATATTT
CGACTTACGGTTCAGATGAGGAATTCCCTTTCCGAATTGGAAGACCGGGATTATGATCGA
CTTATATCTCCCGTGCTCAATGAAAACAATATTTTTTATGATTCAGCGAAAGCTGGGGAC
GCGCTGCCAAAAGATGCCGATGCCAATGGAGCATACTGTATCGCCCTGAAGGGTTTGTAT
GAGATTAAGCAAATTACTGAAAACTGGAAGGAAGATGGCAAGTTTTCTAGAGATAAGCTT
AAGATTAGCAATAAGGACTGGTTTGACTTCATTCAAAATAAAAGGTATCTTAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 84
AGCCCAGCGGCTAAAAAAAAGAAACTGGATGGCAGCGTGGATATGAATAATGGAACAAAT
AATTTTCAAAATTTTATTGGTATCAGTTCATTGCAAAAGACTTTGAGAAATGCTTTGATC
CCGACTGAGACCACACAGCAGTTCATCGTCAAAAATGGCATAATCAAGGAAGACGAACTT
AGGGGTGAGAATAGACAAATATTGAAGGACATCATGGATGACTATTATAGGGGGTTCATT
TCCGAAACGCTCAGTAGTATTGATGACATTGACTGGACTAGTCTTTTCGAGAAAATGGAA
ATTCAGCTTAAGAACGGGGACAATAAAGACACGCTGATCAAGGAGCAAACGGAATATAGG
AAGGCGATCCATAAAAAATTCGCGAATGATGATCGGTTTAAAAACATGTTTAGTGCCAAG
TTGATCAGCGACATACTGCCCGAATTCGTGATCCACAACAATAATTACAGCGCCTCCGAA
AAGGAGGAAAAAACTCAGGTCATTAAATTGTTTAGCCGATTCGCAACGAGTTTCAAAGAT
TATTTTAAGAACCGGGCCAACTGTTTTTCAGCGGATGATATTAGCTCCAGCAGCTGCCAT
CGCATAGTAAATGATAACGCTGAAATCTTTTTTAGCAACGCACTTGTCTACCGGAGGATT
GTAAAATCACTGTCAAATGATGACATTAACAAAATATCTGGAGATATGAAGGACTCACTC
AAAGAAATGAGCCTGGAAGAAATATATTCATACGAAAAATACGGGGAGTTTATTACCCAG
GAAGGTATCAGTTTTTATAATGATATATGTGGAAAAGTTAATTCATTTATGAATCTTTAC
TGTCAAAAAAATAAGGAGAACAAGAATTTGTACAAGCTCCAAAAACTTCATAAACAGATT
CTGTGCATCGCAGACACAAGTTATGAGGTACCGTACAAATTTGAGAGCGACGAAGAAGTT
TATCAGAGTGTGAATGGTTTCCTGGACAATATCTCTTCTAAACACATTGTTGAGAGGCTT
AGGAAGATCGGTGATAATTATAACGGCTATAATCTGGACAAAATTTATATTGTATCAAAG
TTTTATGAATCAGTCTCTCAAAAGACGTATCGGGATTGGGAAACAATTAACACGGCTCTG
GAGATCCACTACAATAACATTCTGCCCGGCAACGGGAAGAGCAAAGCTGATAAGGTCAAG
AAGGCAGTCAAGAACGACCTTCAGAAGAGCATAACAGAAATTAACGAATTGGTCAGTAAC
TACAAACTGTGTAGTGATGACAACATAAAAGCCGAAACATACATCCATGAAATAAGCCAT
ATCCTGAATAACTTCGAAGCCCAAGAACTTAAATACAATCCCGAGATTCATCTTGTCGAA
TCAGAACTCAAGGCGTCCGAGCTCAAAAATGTCCTTGACGTGATAATGAATGCCTTCCAC
TGGTGCAGCGTATTCATGACGGAGGAGTTGGTAGATAAAGACAACAACTTTTATGCCGAA
TTGGAAGAGATTTATGATGAGATTTACCCCGTTATTTCTCTGTACAACTTGGTTCGAAAC
TACGTAACACAAAAACCATACTCAACCAAAAAGATCAAACTCAATTTTGGCATACCTACA
TTGGCTGATGGTTGGTCCAAGTCAAAGGAATATAGCAATAATGCAATAATTCTCATGCGA
GATAACTTGTATTATTTGGGGATCTTTAACGCTAAGAACAAACCAGATAAAAAGATAATC
GAGGGGAACACAAGTGAGAACAAGGGTGATTACAAAAAAATGATTTACAATCTGCTTCCT
GGGCCTAACAAAATGATTCCGAAGGTGTTTCTTAGCTCTAAAACTGGAGTGGAGACGTAT
AAGCCTTCCGCGTACATTCTCGAAGGCTACAAGCAAAATAAGCATATCAAGTCCAGTAAG
GACTTCGACATCACTTTTTGCCACGATCTCATCGATTACTTTAAGAACTGTATCGCAATA
CACCCCGAGTGGAAAAACTTTGGTTTTGATTTTTCAGACACTAGTACCTACGAGGACATT
TCCGGCTTCTATCGAGAAGTCGAACTCCAGGGCTACAAAATCGATTGGACGTACATTTCT
GAGAAGGACATCGACTTGCTCCAAGAGAAAGGTCAACTTTACCTCTTCCAAATTTACAAT
AAAGACTTTTCAAAGAAGAGCACCGGTAATGACAACTTGCATACCATGTATCTGAAGAAC
CTGTTTTCTGAGGAGAACCTCAAGGATATTGTATTGAAGTTGAATGGCGAAGCAGAAATA
TTTTTCCGAAAGTCATCTATCAAGAACCCCATTATACACAAAAAAGGCTCTATCCTGGTG
AACCGGACTTACGAGGCAGAGGAGAAGGATCAATTCGGAAACATACAGATAGTCCGCAAA
AACATCCCTGAGAATATCTATCAGGAACTCTATAAGTACTTCAATGATAAATCAGACAAG
GAGCTTAGCGACGAAGCAGCTAAACTTAAAAACGTGGTTGGCCATCACGAGGCCGCTACC
AACATAGTCAAAGACTACCGCTATACTTATGACAAGTACTTTTTGCACATGCCCATAACA
ATTAATTTCAAAGCTAACAAAACAGGGTTTATAAATGACAGAATCCTCCAATACATCGCC
AAAGAGAAGGACCTCCATGTAATCGGGATTGATAGAGGCGAACGGAACTTGATTTACGTT
AGTGTCATTGATACCTGTGGTAACATTGTCGAACAAAAGTCATTCAACATAGTCAATGGA
TATGATTATCAGATAAAACTCAAGCAACAAGAAGGCGCGAGGCAGATTGCCAGGAAGGAA
TGGAAAGAAATCGGGAAGATCAAGGAGATCAAGGAGGGTTACCTGTCCTTGGTGATACAC
GAGATTTCAAAAATGGTTATAAAATACAATGCCATTATCGCGATGGAGGATTTGTCTTAT
GGATTTAAGAAGGGGAGGTTCAAAGTCGAACGACAAGTCTATCAGAAGTTTGAAACAATG
CTCATTAACAAGCTCAATTACCTTGTTTTCAAGGATATAAGCATCACTGAAAACGGCGGA
CTCCTTAAGGGATATCAGCTGACTTATATCCCCGACAAGCTCAAGAACGTAGGGCACCAA
TGCGGATGCATCTTTTACGTGCCTGCAGCATATACTTCAAAAATTGATCCGACTACTGGC
TTTGTTAACATTTTCAAGTTCAAGGATCTGACGGTAGACGCTAAGAGAGAATTCATAAAA
AAGTTTGACAGCATCAGGTACGATAGTGAAAAGAACCTTTTTTGTTTTACCTTTGACTAC
AATAATTTTATTACGCAAAATACAGTTATGAGCAAATCAAGTTGGAGCGTTTACACATAT
GGCGTTCGGATCAAGCGCAGATTCGTCAATGGTCGCTTCTCAAATGAGAGCGATACAATC
GATATAACGAAGGATATGGAGAAGACGCTTGAGATGACAGATATCAACTGGCGGGACGGA
CATGACCTTAGACAAGACATAATCGATTACGAAATAGTACAGCATATCTTTGAGATTTTT
AGGCTTACAGTTCAGATGCGGAACTCTCTTTCCGAACTGGAGGACCGGGATTATGATCGG
TTGATCTCCCCAGTACTGAACGAAAATAATATCTTTTACGATAGCGCGAAGGCTGGTGAT
GCACTCCCAAAAGACGCTGATGCGAACGGAGCTTATTGCATAGCCCTTAAAGGGCTTTAC
GAGATTAAACAAATAACAGAAAATTGGAAGGAAGATGGCAAATTTTCCCGCGACAAGTTG
AAGATTAGTAACAAAGACTGGTTCGACTTCATTCAGAATAAACGCTACCTCAAACGTCCG
GCAGCGACCAAAAAAGCCGGCCAGGCGAAGAAAAAAAAAGCGTCAGGTAGCGGCGCAGGC
AGCCCGAAAAAGAAACGTAAAGTCGAGGATCCGAAAAAGAAACGTAAGGTTATTCCGGGC
TAA
SEQ ID NO: 85
CCAGCGGCTAAAAAAAAGAAACTGGATGGCAGCGTGGATATGAATAACGGTACCAATAAC
TTCCAGAACTTCATCGGTATTTCTAGCCTGCAAAAGACCCTGCGTAACGCGCTGATTCCG
ACCGAGACTACCCAGCAATTCATCGTGAAAAACGGTATCATTAAGGAAGATGAATTGCGC
GGTGAGAATCGTCAGATTCTGAAAGATATCATGGATGACTACTATCGCGGTTTCATTAGC
GAAACCCTGTCGAGCATCGATGATATCGATTGGACGAGCCTCTTCGAGAAAATGGAAATT
CAACTGAAAAATGGTGACAACAAAGATACCCTGATTAAAGAACAAACGGAATACCGCAAG
GCAATCCATAAAAAGTTTGCGAATGACGACCGTTTTAAGAATATGTTCTCGGCCAAGCTG
ATTTCCGACATCCTGCCAGAGTTCGTCATTCACAACAACAATTACAGCGCAAGCGAGAAA
GAGGAAAAGACTCAGGTCATTAAGCTGTTTAGCCGCTTTGCGACGTCCTTCAAAGACTAC
TTCAAGAATCGTGCGAATTGCTTTAGCGCGGATGACATCTCTAGCTCTAGCTGTCACCGT
ATTGTTAACGACAATGCAGAGATTTTCTTCAGCAACGCCCTGGTGTATCGCCGTATTGTC
AAGTCTCTGAGCAACGACGACATTAACAAGATCAGCGGCGACATGAAAGACAGCCTGAAA
GAAATGTCTCTGGAAGAAATCTACAGCTACGAGAAATATGGTGAGTTTATCACCCAAGAG
GGCATTAGCTTCTACAATGATATCTGTGGTAAGGTTAATAGCTTTATGAATCTGTACTGC
CAGAAGAATAAAGAAAACAAGAACTTGTACAAGCTGCAAAAGCTGCATAAGCAAATTCTG
TGCATCGCCGATACTAGCTATGAAGTTCCGTACAAGTTCGAGTCTGATGAAGAGGTGTAT
CAGTCAGTCAACGGTTTTCTGGATAACATCAGCAGCAAGCACATCGTCGAGCGCCTGCGC
AAGATTGGTGACAACTACAATGGTTATAACCTGGACAAGATCTATATCGTGTCGAAGTTT
TACGAGAGCGTGTCCCAGAAAACGTACCGTGATTGGGAAACGATTAACACGGCCTTGGAA
ATTCACTATAACAATATCCTGCCGGGCAACGGCAAGAGCAAAGCTGACAAAGTCAAAAAA
GCTGTGAAAAACGATCTGCAAAAGTCCATCACCGAGATCAACGAACTGGTTAGCAACTAT
AAGCTGTGTAGCGACGACAACATTAAAGCTGAAACGTATATCCACGAAATCAGCCACATC
CTGAATAACTTTGAGGCACAAGAACTGAAATACAATCCTGAGATCCATCTGGTAGAGAGC
GAGCTGAAGGCAAGCGAGTTGAAAAACGTTCTCGACGTTATCATGAATGCTTTCCACTGG
TGTAGCGTGTTTATGACCGAAGAACTGGTTGACAAAGATAACAATTTCTATGCAGAGCTG
GAAGAAATCTATGATGAAATCTACCCGGTCATCAGCCTGTATAACCTGGTTCGTAACTAC
GTGACGCAGAAGCCGTACAGCACCAAAAAGATCAAGCTGAACTTCGGTATTCCGACCTTG
GCGGACGGTTGGAGCAAATCCAAAGAATACTCCAATAATGCGATTATTCTGATGCGTGAT
AATCTGTACTATCTGGGTATCTTCAATGCGAAGAACAAGCCAGATAAAAAGATTATTGAA
GGCAACACCAGCGAGAATAAAGGCGACTACAAGAAAATGATCTACAACTTATTGCCGGGT
CCGAACAAGATGATCCCGAAAGTTTTTCTGAGCAGCAAGACCGGCGTTGAAACCTATAAG
CCGAGCGCGTACATTTTAGAGGGCTATAAACAAAACAAGCACATCAAGAGCAGCAAAGAT
TTTGATATTACGTTCTGCCACGACCTGATCGACTATTTCAAGAATTGTATTGCGATTCAC
CCTGAGTGGAAGAACTTCGGTTTTGACTTTTCCGATACCTCCACCTATGAAGATATTAGC
GGTTTTTACCGTGAAGTCGAGTTGCAGGGTTATAAGATTGATTGGACTTACATTTCCGAG
AAAGACATCGACCTGTTGCAAGAGAAAGGTCAGCTGTACCTGTTTCAGATCTATAACAAA
GATTTCAGCAAAAAGTCGACGGGCAATGATAATCTGCACACCATGTATCTGAAAAACCTG
TTTAGCGAAGAGAACCTGAAAGACATTGTTCTTAAGCTGAATGGTGAGGCCGAGATCTTC
TTCCGTAAAAGCTCCATTAAGAACCCGATTATCCACAAAAAGGGCTCTATTCTGGTTAAC
CGCACGTACGAAGCGGAAGAGAAAGATCAATTTGGTAACATCCAGATCGTGCGTAAGAAT
ATCCCGGAGAACATTTACCAAGAACTGTATAAGTATTTCAATGACAAGAGCGATAAAGAA
TTGAGCGATGAAGCGGCAAAGCTGAAAAACGTCGTTGGCCACCACGAAGCCGCGACGAAT
ATCGTGAAAGATTATCGTTACACCTACGACAAGTACTTTCTGCACATGCCGATCACCATC
AATTTCAAAGCGAATAAAACGGGTTTTATCAATGACCGTATCCTGCAGTACATTGCGAAA
GAAAAAGATTTACACGTGATTGGTATTGATCGCGGCGAGCGCAATCTGATTTACGTCAGC
GTTATCGACACGTGCGGCAATATTGTGGAGCAGAAAAGCTTCAATATCGTCAATGGTTAC
GACTACCAGATCAAACTGAAGCAACAAGAGGGCGCCCGCCAGATTGCGCGTAAAGAGTGG
AAAGAAATCGGTAAGATTAAAGAAATCAAGGAAGGCTACCTGTCCCTGGTGATCCATGAA
ATCAGCAAAATGGTGATCAAGTACAACGCTATCATTGCGATGGAAGATCTGAGCTACGGT
TTTAAAAAGGGTCGCTTCAAAGTTGAGCGTCAAGTGTATCAGAAATTTGAGACTATGCTG
ATTAACAAGTTGAACTATCTGGTTTTTAAAGACATCAGCATTACCGAGAATGGTGGCCTG
CTGAAGGGTTATCAACTGACCTATATTCCTGACAAGTTGAAAAATGTTGGTCATCAGTGT
GGTTGCATTTTCTACGTACCGGCAGCGTACACGAGCAAGATTGACCCGACCACGGGTTTC
GTTAACATTTTCAAGTTTAAAGATTTGACCGTGGACGCCAAGCGTGAGTTCATTAAAAAG
TTCGACAGCATCAGATACGACTCTGAGAAGAATCTGTTCTGCTTTACGTTCGACTACAAT
AACTTCATTACCCAAAATACCGTTATGAGCAAAAGCTCCTGGAGCGTGTACACGTACGGC
GTCCGTATCAAGCGTCGTTTTGTGAATGGTCGCTTTTCCAACGAATCTGACACCATTGAC
ATTACCAAAGATATGGAAAAGACCCTTGAGATGACCGACATTAATTGGCGTGATGGCCAT
GACTTGCGCCAAGACATTATCGACTACGAAATTGTTCAGCACATCTTTGAGATTTTTCGT
CTGACGGTCCAGATGCGCAACTCGCTGAGCGAGTTGGAAGATCGTGACTATGACCGTCTG
ATTAGCCCGGTGCTGAATGAAAACAATATCTTCTATGATAGCGCAAAGGCCGGTGACGCG
CTGCCGAAAGATGCGGATGCTAACGGTGCATACTGCATTGCACTGAAGGGTCTGTACGAA
ATCAAACAGATCACCGAGAATTGGAAAGAGGATGGTAAGTTTAGCCGTGATAAGCTGAAG
ATTAGCAATAAAGACTGGTTCGACTTTATTCAAAACAAGCGCTATCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 86
CCAGCGGCTAAAAAAAAGAAACTGGATGGCAGCGTGGATATGAACAACGGAACAAATAAT
TTTCAGAACTTTATTGGGATCAGTTCGCTTCAGAAAACGCTTCGTAATGCTCTGATTCCC
ACAGAAACCACTCAGCAGTTTATCGTAAAGAATGGCATTATCAAGGAGGATGAATTACGC
GGCGAGAACCGCCAAATCTTAAAAGATATCATGGACGACTACTACCGCGGTTTCATTAGC
GAAACTCTTAGTTCAATTGACGACATTGACTGGACGTCCTTGTTCGAAAAGATGGAGATT
CAATTAAAGAACGGTGATAACAAGGATACGTTGATTAAAGAACAGACGGAGTACCGTAAG
GCTATCCACAAAAAATTTGCAAACGACGACCGCTTTAAAAATATGTTTAGCGCAAAATTA
ATCTCCGACATCCTGCCTGAATTCGTCATCCATAACAATAACTATAGCGCCTCGGAAAAA
GAAGAAAAAACGCAGGTTATTAAACTTTTCTCGCGCTTTGCAACAAGCTTTAAGGATTAC
TTCAAAAATCGCGCCAATTGTTTTTCAGCCGACGACATTAGCTCCAGTTCCTGCCACCGT
ATTGTGAATGACAACGCTGAGATTTTTTTTTCCAATGCGCTGGTTTATCGTCGTATTGTT
AAGAGCCTTAGTAACGACGACATTAATAAAATTAGCGGTGATATGAAGGATAGCTTGAAA
GAAATGAGTCTGGAAGAGATCTATAGTTACGAGAAGTACGGCGAATTTATTACCCAGGAG
GGCATTTCATTTTACAATGATATCTGTGGAAAAGTCAACTCCTTTATGAACTTGTATTGC
CAAAAGAATAAAGAAAACAAAAACCTGTACAAACTGCAAAAGTTACACAAGCAGATTTTG
TGTATCGCAGACACGTCATACGAAGTACCGTACAAGTTTGAGTCCGATGAAGAAGTGTAC
CAAAGCGTTAATGGCTTTTTGGATAACATTTCGAGCAAACATATCGTAGAGCGTTTGCGT
AAGATTGGTGATAATTACAACGGTTACAATTTAGACAAAATCTATATCGTCTCTAAGTTT
TACGAAAGTGTTTCTCAGAAAACTTACCGCGATTGGGAGACGATCAACACTGCGCTGGAG
ATTCATTACAATAATATCCTTCCAGGTAACGGTAAAAGCAAAGCTGATAAGGTGAAAAAG
GCGGTTAAAAATGACCTTCAAAAGTCTATCACAGAAATCAACGAATTGGTCAGCAATTAT
AAGCTTTGCAGTGACGATAACATTAAGGCCGAGACTTACATCCATGAGATCTCTCACATT
CTTAATAATTTTGAAGCGCAAGAGCTGAAATACAATCCTGAAATCCATCTGGTCGAAAGT
GAATTAAAAGCCTCCGAATTAAAAAATGTCTTGGACGTGATCATGAATGCGTTCCATTGG
TGCTCAGTTTTTATGACGGAAGAGTTGGTGGACAAAGACAACAATTTTTACGCCGAGCTT
GAGGAAATTTACGACGAAATTTACCCCGTTATTTCGTTATACAACCTTGTGCGTAATTAC
GTTACACAAAAGCCCTATTCGACAAAGAAAATCAAGTTAAATTTCGGGATTCCCACATTA
GCTGATGGATGGTCCAAATCCAAAGAATACTCGAATAACGCTATCATCCTTATGCGTGAT
AATTTGTACTACTTAGGCATCTTCAATGCGAAGAACAAACCTGACAAGAAAATTATCGAA
GGAAACACTTCGGAGAACAAAGGTGATTATAAAAAGATGATCTACAACTTGCTTCCCGGG
CCAAACAAAATGATTCCCAAGGTATTTTTGAGTTCTAAAACCGGTGTCGAAACTTACAAA
CCAAGTGCTTATATTTTGGAAGGATACAAACAGAACAAACATATCAAGTCTTCGAAAGAC
TTCGATATTACGTTCTGCCACGATCTGATCGATTACTTCAAGAACTGTATTGCTATTCAC
CCCGAGTGGAAGAACTTTGGATTTGATTTCTCCGACACGTCCACTTATGAAGATATCTCT
GGCTTCTATCGCGAGGTTGAATTACAAGGGTATAAGATTGACTGGACTTATATTTCGGAG
AAGGATATCGATCTTTTGCAAGAAAAAGGGCAACTTTATTTATTTCAGATCTATAACAAG
GACTTTTCAAAAAAGAGCACTGGAAATGACAATCTGCATACCATGTACCTTAAGAACCTG
TTCTCGGAAGAGAACCTGAAGGACATTGTACTTAAACTGAATGGAGAGGCAGAGATCTTC
TTTCGCAAATCAAGCATTAAGAACCCAATTATTCACAAAAAGGGGAGTATCTTAGTAAAT
CGCACATATGAGGCTGAGGAAAAAGATCAGTTTGGTAACATTCAGATCGTGCGTAAGAAC
ATTCCTGAAAATATCTATCAGGAACTTTATAAGTATTTCAACGATAAAAGTGATAAAGAG
CTGAGTGACGAAGCGGCTAAACTTAAGAATGTTGTGGGACACCATGAGGCAGCAACCAAT
ATTGTGAAGGATTATCGCTATACGTACGACAAATACTTTTTACACATGCCCATCACTATT
AATTTTAAAGCTAATAAGACTGGCTTCATTAACGATCGCATCCTGCAGTACATTGCTAAG
GAAAAGGATCTTCACGTTATCGGTATCGATCGCGGGGAGCGTAATCTTATCTACGTCTCT
GTCATTGACACGTGTGGCAATATTGTGGAGCAAAAGTCCTTCAATATTGTTAACGGCTAT
GACTATCAGATTAAATTGAAACAGCAGGAAGGTGCGCGTCAGATTGCCCGCAAGGAATGG
AAGGAAATTGGCAAGATCAAAGAAATTAAGGAGGGCTACTTAAGCTTAGTAATTCACGAA
ATTAGTAAAATGGTTATCAAATACAACGCCATCATCGCGATGGAGGATCTTTCGTACGGG
TTTAAGAAAGGTCGTTTTAAAGTGGAGCGTCAGGTGTACCAGAAATTTGAAACTATGCTT
ATTAACAAACTTAACTACCTGGTTTTCAAGGATATCAGTATTACTGAAAACGGGGGGCTG
TTAAAAGGGTATCAATTAACTTACATTCCAGACAAATTAAAGAACGTTGGACATCAGTGT
GGCTGCATTTTTTATGTACCAGCTGCATACACTTCAAAGATCGATCCTACGACTGGGTTC
GTGAACATTTTTAAGTTTAAAGACTTGACGGTAGATGCCAAGCGCGAATTCATCAAGAAA
TTCGACAGCATTCGCTACGACTCTGAGAAAAATCTTTTCTGTTTCACATTCGATTATAAC
AATTTCATTACGCAGAACACAGTAATGTCCAAGTCTTCTTGGAGTGTTTATACATATGGT
GTCCGCATTAAGCGCCGTTTCGTCAACGGCCGCTTCAGTAATGAGAGCGATACTATTGAC
ATCACAAAAGACATGGAAAAAACACTGGAAATGACCGACATCAATTGGCGTGACGGCCAT
GACTTACGTCAGGATATCATTGATTATGAGATCGTTCAACACATCTTCGAAATCTTTCGC
TTGACTGTTCAAATGCGCAATTCCTTGTCGGAATTGGAGGACCGTGATTATGACCGCTTA
ATTTCCCCCGTCTTAAATGAAAACAATATTTTTTATGACTCTGCAAAAGCTGGAGATGCT
CTGCCGAAAGACGCCGATGCAAATGGGGCATATTGCATTGCTTTAAAGGGGCTTTACGAG
ATCAAGCAAATCACCGAAAACTGGAAAGAGGATGGAAAGTTTTCGCGTGATAAACTGAAG
ATCTCTAACAAAGACTGGTTCGACTTTATCCAGAACAAGCGTTATTTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 87
CCAGCGGCTAAAAAAAAGAAACTGGATGGCAGCGTGGATATGAACAACGGCACCAATAAC
TTCCAAAACTTCATCGGGATCTCTAGCCTTCAGAAGACGCTTCGCAATGCTCTTATCCCA
ACTGAGACCACTCAACAATTTATTGTGAAGAATGGAATTATTAAAGAGGACGAACTGCGT
GGCGAGAATCGTCAGATCTTAAAGGACATTATGGATGATTATTACCGTGGATTCATCTCC
GAAACATTATCGTCGATCGATGATATCGATTGGACTTCTCTGTTCGAGAAAATGGAAATT
CAATTGAAAAACGGAGATAATAAAGATACGCTTATCAAAGAACAGACGGAATATCGTAAA
GCGATTCATAAGAAATTCGCAAATGACGATCGTTTCAAAAATATGTTCAGTGCCAAGCTT
ATTTCGGACATTTTACCTGAATTTGTAATTCATAATAATAACTACTCAGCAAGTGAGAAG
GAGGAGAAAACCCAAGTTATTAAACTGTTCTCTCGTTTCGCAACGTCCTTTAAAGATTAC
TTTAAAAACCGCGCGAATTGCTTTAGCGCTGACGACATTTCCAGCTCATCCTGTCATCGC
ATCGTAAACGACAATGCGGAAATCTTCTTCAGCAACGCCCTGGTTTACCGCCGCATCGTC
AAAAGCTTATCGAATGACGACATCAATAAGATCTCAGGAGATATGAAGGACTCGCTTAAG
GAGATGTCTCTGGAGGAAATTTATAGTTACGAAAAGTATGGAGAGTTCATTACCCAGGAG
GGAATCTCGTTCTACAATGACATTTGCGGGAAGGTGAACTCCTTCATGAACTTATACTGC
CAGAAAAACAAAGAGAACAAAAATCTGTATAAATTGCAGAAATTACATAAACAGATTCTT
TGTATTGCTGACACTTCCTACGAAGTACCCTATAAATTCGAGTCAGATGAAGAAGTATAC
CAGTCCGTGAACGGATTTCTGGACAATATCTCCTCAAAACACATCGTGGAACGCTTACGT
AAAATTGGCGATAATTATAATGGTTACAATCTTGACAAAATTTATATCGTATCTAAATTT
TACGAGAGTGTGAGCCAAAAGACCTACCGCGACTGGGAGACCATCAACACAGCTTTAGAA
ATTCACTATAATAATATCTTACCCGGCAATGGTAAGAGCAAGGCTGACAAGGTAAAAAAG
GCCGTCAAGAATGATTTGCAGAAATCTATTACAGAAATTAATGAGTTAGTCTCCAACTAT
AAGCTTTGTTCCGACGATAACATCAAAGCTGAGACATATATTCATGAGATTAGTCACATT
CTTAACAACTTCGAGGCCCAGGAACTTAAGTACAATCCTGAAATTCATCTTGTCGAGTCT
GAGCTGAAAGCTAGTGAATTGAAAAATGTTTTAGACGTTATTATGAACGCATTCCACTGG
TGCTCTGTGTTTATGACAGAAGAACTGGTCGACAAGGACAATAACTTCTATGCCGAACTT
GAGGAAATCTACGATGAAATTTACCCTGTAATCTCCTTGTATAATCTTGTACGTAATTAC
GTCACTCAAAAACCTTACAGCACGAAAAAAATTAAATTGAACTTCGGGATTCCTACACTT
GCCGACGGGTGGTCTAAATCCAAGGAATATAGCAACAATGCCATTATTTTAATGCGCGAC
AATCTTTACTATTTAGGAATTTTTAACGCTAAGAACAAGCCCGATAAAAAGATTATTGAA
GGAAACACGTCTGAAAATAAGGGCGACTACAAAAAGATGATTTATAACCTTTTGCCCGGT
CCAAACAAAATGATCCCAAAGGTATTCCTGTCATCCAAAACAGGGGTTGAGACATATAAG
CCCAGCGCATATATTCTGGAAGGATACAAACAGAATAAACATATCAAAAGCAGCAAAGAT
TTTGACATTACTTTTTGCCACGATTTAATCGACTACTTCAAAAACTGTATCGCTATCCAC
CCTGAATGGAAGAATTTCGGATTTGATTTCTCAGATACAAGTACGTATGAGGATATCAGC
GGTTTCTATCGCGAAGTTGAACTTCAAGGGTATAAAATTGACTGGACCTACATTAGTGAG
AAGGACATCGACCTGTTACAGGAAAAAGGCCAATTGTACTTGTTTCAGATCTACAATAAG
GATTTCTCAAAAAAATCGACCGGCAATGATAACTTGCACACCATGTACCTGAAGAACCTT
TTTTCGGAGGAAAACCTTAAAGACATTGTCCTGAAGTTGAATGGAGAAGCGGAGATTTTC
TTTCGTAAGTCTTCCATTAAAAATCCAATTATTCATAAGAAGGGCAGCATCCTTGTGAAC
CGTACGTACGAGGCGGAAGAGAAGGACCAATTCGGTAACATTCAAATCGTCCGCAAGAAC
ATCCCTGAAAATATTTATCAGGAGCTTTACAAGTATTTCAATGATAAGTCCGACAAGGAA
TTATCAGATGAGGCTGCGAAGTTGAAAAATGTTGTTGGTCATCACGAGGCGGCGACGAAT
ATTGTAAAGGATTATCGCTACACTTATGACAAGTACTTTCTGCACATGCCGATCACCATT
AATTTCAAGGCGAACAAAACAGGATTTATTAATGACCGCATCTTACAATACATTGCCAAA
GAAAAGGACTTACACGTTATTGGCATTGATCGTGGAGAACGCAACTTAATCTACGTAAGC
GTTATTGACACTTGCGGGAATATCGTAGAACAAAAGAGCTTCAACATCGTGAATGGTTAC
GATTACCAGATCAAGCTTAAGCAGCAGGAGGGAGCGCGCCAGATCGCGCGCAAGGAATGG
AAGGAGATTGGTAAGATCAAGGAAATCAAGGAAGGTTATCTGTCCTTGGTAATCCACGAA
ATTTCGAAAATGGTTATCAAATACAATGCTATTATTGCAATGGAGGACTTGTCCTACGGC
TTTAAAAAAGGACGCTTTAAGGTGGAGCGCCAGGTTTATCAAAAGTTTGAAACAATGCTG
ATTAACAAGCTGAACTATTTGGTCTTTAAAGATATCTCCATCACCGAAAATGGTGGGCTT
TTGAAAGGCTATCAACTTACATATATCCCTGATAAGCTTAAGAATGTGGGTCATCAGTGC
GGGTGCATTTTTTATGTTCCTGCAGCCTACACGTCCAAAATCGATCCTACAACTGGATTT
GTTAATATCTTCAAATTTAAGGATCTTACCGTCGACGCGAAGCGCGAATTTATCAAGAAA
TTCGATAGTATTCGTTATGATTCCGAAAAAAACCTTTTCTGTTTCACCTTTGATTATAAT
AACTTTATCACGCAAAATACTGTCATGAGCAAATCGAGTTGGTCTGTGTACACTTACGGA
GTACGCATCAAGCGTCGTTTTGTTAATGGGCGCTTCAGTAACGAGTCAGACACGATTGAT
ATCACAAAAGATATGGAGAAAACGCTGGAGATGACAGACATCAATTGGCGCGATGGTCAT
GACTTACGTCAAGACATTATCGATTATGAAATTGTCCAGCATATCTTTGAGATCTTTCGT
TTGACTGTTCAGATGCGCAACAGCCTGTCAGAATTGGAGGATCGTGACTATGATCGCCTT
ATTTCTCCCGTCTTAAATGAGAACAATATCTTCTACGACTCAGCCAAGGCTGGAGATGCA
CTGCCAAAAGACGCCGACGCAAATGGGGCCTACTGTATTGCATTGAAGGGGTTGTACGAG
ATCAAACAGATTACAGAAAATTGGAAGGAGGACGGTAAGTTCTCTCGTGATAAGCTGAAG
ATTTCTAACAAAGACTGGTTCGATTTCATTCAGAACAAACGTTACCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 88
CCAGCGGCTAAAAAAAAGAAACTGGATGGCAGCGTGGATATGAACAACGGTACCAATAAC
TTTCAGAATTTCATTGGAATCAGCAGCTTACAGAAAACCCTGCGCAATGCACTTATCCCC
ACTGAGACAACCCAGCAGTTCATTGTAAAGAACGGGATTATTAAAGAAGATGAGCTTCGC
GGGGAGAATCGTCAGATCTTAAAGGATATTATGGACGATTACTACCGTGGCTTCATTTCG
GAGACGCTGTCGTCGATCGACGACATCGACTGGACATCCTTGTTTGAAAAGATGGAAATC
CAACTGAAGAATGGCGATAACAAGGACACGTTAATCAAAGAGCAGACGGAATACCGTAAA
GCTATCCACAAAAAGTTCGCTAATGACGACCGCTTTAAGAACATGTTCTCAGCAAAACTT
ATTAGCGATATTTTACCTGAATTTGTCATCCACAATAACAATTACTCCGCGAGTGAAAAA
GAGGAGAAAACCCAGGTGATTAAGCTGTTTTCCCGTTTTGCAACCAGTTTCAAGGACTAT
TTTAAGAATCGTGCTAATTGTTTCTCTGCAGACGACATTTCCTCGTCGTCCTGCCATCGC
ATTGTTAATGATAATGCTGAAATCTTTTTTTCAAACGCACTTGTGTATCGTCGCATTGTC
AAAAGCTTAAGTAATGACGATATCAATAAGATCTCAGGAGACATGAAGGACTCCCTGAAA
GAAATGTCATTGGAAGAAATTTACTCTTATGAAAAGTATGGAGAATTTATTACGCAGGAG
GGTATCAGCTTCTATAACGACATTTGTGGTAAAGTGAACAGCTTTATGAATCTTTATTGT
CAAAAGAATAAAGAGAACAAAAATCTGTACAAGCTGCAGAAATTGCATAAACAAATTCTG
TGCATTGCAGATACTTCGTATGAGGTTCCTTACAAATTCGAGTCGGATGAGGAGGTGTAT
CAAAGCGTAAACGGATTTTTGGATAACATTAGTAGTAAGCATATTGTGGAACGCCTTCGC
AAGATTGGTGACAACTATAACGGATACAACTTAGACAAGATCTATATTGTCTCGAAGTTT
TACGAAAGTGTTTCCCAAAAGACTTATCGCGACTGGGAGACAATCAACACTGCGCTGGAA
ATTCACTATAACAATATCTTGCCGGGGAACGGAAAAAGTAAGGCAGATAAGGTGAAGAAA
GCAGTCAAAAATGATCTGCAAAAAAGCATTACTGAAATTAACGAACTTGTGTCAAATTAC
AAATTGTGTTCGGATGACAATATTAAAGCGGAAACGTATATCCACGAGATCTCGCACATT
CTTAATAATTTCGAGGCGCAGGAATTAAAGTATAATCCTGAGATCCATTTGGTGGAATCA
GAACTTAAAGCTAGTGAACTGAAAAATGTCCTGGACGTTATTATGAATGCATTTCACTGG
TGTTCTGTCTTTATGACAGAAGAACTTGTCGACAAAGACAACAACTTTTATGCGGAATTA
GAAGAGATTTACGACGAAATTTATCCCGTTATTTCGTTATATAATTTAGTTCGTAATTAC
GTGACTCAGAAACCCTACAGCACAAAAAAGATTAAATTAAACTTTGGGATTCCGACTCTT
GCTGATGGATGGAGCAAGTCCAAGGAGTACTCTAATAACGCCATTATCTTGATGCGTGAC
AACCTGTACTACCTGGGCATTTTTAACGCTAAAAACAAACCCGACAAAAAGATCATTGAA
GGGAACACCTCGGAAAATAAGGGGGACTATAAAAAAATGATCTACAATCTGTTGCCAGGC
CCAAATAAGATGATCCCAAAGGTTTTTTTATCTTCCAAAACTGGCGTAGAAACTTACAAG
CCGAGCGCATACATCCTTGAAGGATATAAACAAAACAAACATATCAAAAGTTCAAAGGAC
TTCGATATTACGTTCTGCCATGATTTAATCGATTATTTCAAGAATTGCATCGCGATTCAC
CCAGAGTGGAAAAACTTTGGGTTTGATTTTTCAGACACCAGCACTTACGAGGATATTAGT
GGATTCTATCGTGAGGTTGAACTGCAGGGCTATAAAATTGACTGGACCTATATTTCTGAA
AAAGATATTGATCTGCTTCAGGAGAAAGGCCAATTGTACTTATTTCAAATCTATAACAAG
GATTTCTCCAAGAAGTCCACGGGTAATGACAACTTACACACAATGTATCTGAAGAATCTG
TTTAGTGAGGAGAACTTGAAGGACATTGTGCTGAAGCTTAATGGCGAGGCCGAAATCTTT
TTTCGTAAGTCCTCCATTAAAAACCCTATTATCCATAAGAAAGGGAGTATTCTTGTCAAC
CGCACGTATGAGGCCGAAGAAAAGGACCAATTCGGAAACATCCAAATTGTCCGTAAAAAT
ATTCCTGAGAACATTTACCAGGAGCTTTACAAGTATTTCAACGACAAGAGTGATAAAGAA
CTTTCAGATGAGGCGGCGAAACTGAAGAATGTAGTGGGGCACCACGAAGCTGCCACGAAT
ATTGTAAAGGATTACCGTTACACCTACGACAAGTACTTTTTGCATATGCCCATCACAATT
AATTTTAAGGCCAATAAAACTGGTTTTATCAACGATCGTATCTTACAGTACATTGCTAAG
GAAAAAGATCTGCACGTTATCGGTATCGATCGCGGGGAACGCAATCTGATTTATGTTAGT
GTGATTGACACGTGCGGAAATATTGTTGAGCAGAAGAGCTTTAATATCGTAAATGGATAT
GACTATCAAATTAAACTGAAGCAACAGGAAGGGGCCCGCCAGATTGCCCGCAAGGAGTGG
AAAGAAATTGGAAAGATCAAGGAGATTAAAGAAGGGTACCTTTCCCTTGTTATCCACGAA
ATCTCGAAAATGGTGATCAAGTACAATGCCATTATTGCTATGGAGGATCTGTCATATGGG
TTTAAGAAAGGCCGCTTTAAGGTGGAACGTCAGGTTTACCAGAAGTTTGAGACCATGCTT
ATCAATAAGCTGAATTATCTTGTCTTCAAAGACATCTCAATCACAGAGAACGGCGGGCTG
TTAAAAGGATATCAGCTGACCTATATCCCCGACAAACTGAAAAATGTCGGGCACCAATGC
GGCTGTATTTTCTACGTGCCCGCTGCATACACATCTAAAATTGACCCAACGACTGGATTC
GTAAATATTTTTAAGTTTAAGGATCTTACGGTAGATGCAAAGCGCGAATTTATCAAGAAA
TTTGATAGTATCCGTTACGACAGCGAGAAAAACTTATTTTGTTTTACGTTCGATTATAAC
AACTTCATCACGCAAAATACCGTCATGTCAAAATCTTCCTGGTCAGTCTATACGTATGGC
GTCCGTATCAAGCGCCGCTTCGTCAACGGGCGTTTTTCAAACGAGTCAGATACCATCGAT
ATCACCAAAGATATGGAAAAAACATTGGAGATGACGGACATCAATTGGCGCGATGGTCAT
GACTTACGCCAGGACATTATTGACTACGAAATCGTACAACATATTTTTGAGATTTTCCGT
CTGACCGTGCAAATGCGCAACTCATTATCCGAACTTGAGGATCGTGATTACGACCGCTTG
ATCAGTCCTGTTCTGAACGAGAATAATATTTTTTACGACAGTGCCAAGGCGGGAGACGCA
CTGCCCAAGGACGCTGACGCTAACGGAGCTTATTGTATTGCGTTGAAGGGACTTTACGAA
ATCAAGCAAATCACTGAAAACTGGAAGGAGGATGGTAAATTCTCACGCGACAAGTTGAAA
ATTTCGAACAAGGACTGGTTCGATTTCATCCAAAACAAGCGTTATTTAAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 89
CCAGCGGCTAAAAAAAAGAAACTGGATGGCAGCGTGGATATGAACAACGGGACTAATAAC
TTCCAGAACTTCATCGGTATTTCATCATTACAAAAAACGCTTCGTAACGCCTTGATCCCA
ACAGAAACGACCCAACAATTTATTGTAAAAAACGGCATCATCAAAGAAGACGAACTGCGT
GGCGAAAATCGCCAAATTTTGAAGGACATTATGGATGACTATTATCGTGGGTTTATCTCG
GAGACATTATCCTCCATCGACGACATTGATTGGACGAGTCTTTTTGAGAAAATGGAGATC
CAGCTTAAAAATGGTGATAACAAGGATACATTGATCAAGGAGCAAACCGAGTACCGCAAG
GCCATCCATAAGAAGTTCGCAAATGACGACCGCTTCAAAAATATGTTTAGTGCCAAATTG
ATCTCGGATATCCTTCCTGAGTTCGTAATTCACAACAATAATTATAGCGCATCCGAAAAG
GAGGAAAAGACTCAAGTCATTAAGCTTTTCAGTCGCTTTGCTACCTCGTTTAAGGACTAT
TTCAAGAACCGCGCGAACTGCTTCTCAGCGGATGACATTTCTTCCTCGTCGTGTCACCGC
ATCGTGAATGATAATGCGGAGATCTTCTTTAGTAATGCCTTGGTATACCGCCGCATTGTT
AAATCCCTGTCTAACGACGATATCAATAAGATCTCAGGAGATATGAAGGATAGCCTTAAA
GAAATGTCTCTGGAAGAAATTTACTCCTATGAAAAGTACGGTGAGTTTATCACCCAAGAG
GGGATTAGCTTTTATAACGATATCTGCGGGAAGGTGAATTCGTTTATGAACCTTTATTGT
CAAAAGAATAAGGAGAATAAGAACTTATATAAGCTTCAGAAACTGCATAAACAAATCTTA
TGCATTGCCGATACTAGCTATGAAGTTCCGTATAAATTCGAGAGCGATGAAGAAGTTTAT
CAGAGCGTCAATGGGTTCTTGGATAACATTTCATCAAAACACATCGTGGAACGTCTGCGT
AAGATTGGGGATAACTACAACGGATATAATCTTGACAAAATTTATATTGTATCTAAATTC
TATGAGTCGGTGAGTCAAAAGACCTACCGTGATTGGGAAACAATCAATACCGCGTTAGAA
ATCCACTATAACAACATTCTGCCAGGGAATGGTAAAAGTAAAGCGGACAAAGTCAAGAAG
GCTGTGAAGAACGATCTGCAAAAGAGTATTACAGAGATTAACGAATTAGTCTCCAATTAT
AAGTTATGCTCGGACGATAACATTAAGGCGGAGACGTATATTCATGAGATTTCGCATATT
CTTAACAACTTCGAGGCACAAGAGCTTAAGTATAACCCAGAGATTCACCTTGTCGAATCG
GAGCTGAAGGCATCGGAATTAAAAAATGTCTTAGATGTAATCATGAACGCGTTCCATTGG
TGCAGTGTTTTCATGACTGAGGAGTTAGTTGACAAGGACAATAACTTCTACGCAGAATTA
GAAGAGATCTATGATGAGATTTATCCAGTGATTTCGCTGTATAATCTGGTACGTAATTAC
GTCACTCAAAAGCCCTACTCAACAAAAAAAATTAAGCTGAACTTCGGAATTCCGACTCTG
GCCGACGGGTGGTCCAAGTCAAAGGAGTATTCTAATAATGCTATCATCCTGATGCGCGAT
AACTTATACTATTTGGGAATTTTCAATGCCAAAAATAAACCAGATAAAAAGATTATCGAA
GGTAATACAAGCGAGAATAAGGGTGACTATAAGAAAATGATTTACAATCTTCTTCCAGGC
CCTAACAAGATGATTCCCAAAGTTTTTTTGTCCAGTAAAACAGGGGTCGAAACTTACAAG
CCCAGTGCCTATATCCTTGAAGGGTACAAGCAGAATAAGCACATCAAATCCTCGAAAGAC
TTTGATATTACATTTTGTCATGACTTAATCGATTATTTTAAGAACTGTATCGCAATCCAT
CCAGAATGGAAGAACTTCGGGTTTGATTTCTCTGATACTTCCACGTATGAGGATATTTCC
GGGTTCTACCGCGAAGTAGAGCTTCAGGGCTATAAAATTGACTGGACATATATTTCAGAA
AAAGACATCGATCTGTTACAAGAAAAAGGACAGTTGTATCTGTTTCAAATCTATAATAAG
GATTTCTCCAAAAAGTCAACTGGAAATGATAACTTACATACAATGTATCTGAAAAATCTT
TTTAGTGAAGAGAATTTGAAGGATATCGTGCTGAAGTTAAATGGCGAAGCAGAGATCTTC
TTCCGCAAGTCCTCGATCAAGAATCCTATCATCCACAAGAAAGGTAGTATTCTGGTTAAC
CGCACGTACGAGGCCGAGGAAAAAGACCAGTTCGGTAATATCCAGATTGTACGTAAGAAT
ATTCCTGAAAATATTTACCAGGAATTATACAAGTATTTTAACGACAAATCGGATAAGGAG
CTTTCAGATGAGGCCGCAAAGTTGAAGAACGTCGTAGGACACCATGAGGCCGCTACGAAT
ATCGTCAAGGACTACCGCTATACGTATGACAAGTACTTCCTGCACATGCCTATTACTATC
AATTTCAAAGCTAATAAAACAGGATTCATCAATGATCGTATCCTTCAGTACATTGCCAAA
GAAAAAGATCTGCACGTAATCGGAATCGACCGTGGCGAACGTAATCTGATTTACGTATCA
GTTATCGACACATGTGGTAACATCGTGGAGCAGAAATCTTTTAACATTGTTAACGGCTAT
GATTATCAGATTAAGCTTAAACAGCAGGAGGGGGCACGCCAAATCGCTCGTAAAGAATGG
AAGGAGATTGGAAAGATTAAAGAGATTAAAGAGGGGTACCTTTCGCTGGTTATTCACGAA
ATTTCCAAGATGGTGATTAAGTACAATGCAATCATCGCGATGGAAGATCTTAGTTACGGA
TTCAAAAAGGGACGCTTCAAAGTTGAGCGTCAGGTCTACCAGAAATTTGAAACGATGCTG
ATTAACAAATTGAATTACTTGGTATTCAAAGATATCTCAATTACTGAAAATGGTGGCTTA
TTAAAGGGTTACCAGCTTACCTATATCCCGGATAAGCTGAAGAACGTGGGCCATCAATGC
GGCTGCATCTTTTACGTCCCTGCCGCATATACCTCTAAAATTGACCCCACCACCGGATTC
GTAAATATTTTTAAATTCAAGGACCTGACGGTGGACGCCAAGCGCGAATTCATCAAAAAA
TTCGACTCAATCCGCTATGATTCCGAAAAAAATCTTTTCTGCTTTACGTTCGATTATAAT
AACTTCATTACCCAAAACACGGTGATGTCAAAATCGTCCTGGAGCGTGTATACTTATGGA
GTGCGTATCAAGCGCCGCTTTGTTAATGGGCGCTTCAGTAACGAAAGCGATACCATCGAC
ATTACCAAAGACATGGAGAAGACGCTTGAAATGACGGATATCAATTGGCGTGACGGACAC
GATCTTCGTCAGGATATCATCGACTACGAGATTGTGCAACATATCTTTGAGATTTTCCGT
TTAACTGTTCAAATGCGTAACTCCTTGTCCGAATTGGAAGACCGTGATTACGACCGCTTG
ATTTCACCAGTGCTTAACGAGAATAACATCTTCTACGACTCCGCCAAAGCAGGCGATGCC
CTGCCAAAGGACGCTGATGCAAATGGTGCATACTGTATCGCGTTGAAGGGCTTATACGAG
ATTAAGCAAATCACCGAAAATTGGAAAGAGGATGGAAAGTTCAGTCGCGATAAGCTGAAG
ATCTCTAATAAAGATTGGTTTGACTTTATCCAGAACAAACGTTATTTAAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 90
CCAGCGGCTAAAAAAAAGAAACTGGATGGCAGCGTGGATATGAACAACGGTACCAATAAT
TTCCAAAATTTCATCGGAATCTCATCCTTGCAAAAAACCTTGCGCAATGCTTTGATCCCC
ACCGAAACCACGCAGCAGTTCATCGTGAAAAACGGCATTATCAAAGAGGATGAGTTGCGC
GGGGAAAACCGTCAAATTCTTAAGGATATCATGGACGATTACTACCGTGGGTTTATCAGT
GAGACCCTGTCAAGCATTGACGACATTGACTGGACCAGCTTATTTGAGAAGATGGAGATT
CAATTAAAGAACGGGGACAATAAGGACACGCTTATCAAAGAGCAGACAGAATACCGTAAA
GCGATTCATAAGAAATTTGCAAATGACGATCGCTTCAAGAACATGTTTTCAGCAAAATTA
ATCAGCGACATCCTTCCCGAATTTGTGATTCATAATAACAACTATTCGGCTAGCGAAAAA
GAGGAGAAAACTCAGGTTATTAAGCTTTTCTCGCGTTTTGCCACTTCGTTCAAAGACTAT
TTTAAGAATCGCGCAAACTGCTTTTCGGCTGATGATATTTCCAGTTCTAGCTGCCATCGT
ATCGTTAACGATAATGCTGAGATTTTCTTCTCTAATGCCCTGGTGTATCGTCGTATCGTT
AAATCTTTGAGCAACGACGATATTAATAAGATTTCAGGCGACATGAAGGATTCTTTAAAG
GAGATGTCTTTAGAAGAGATTTATTCCTATGAGAAATATGGCGAGTTTATCACCCAAGAA
GGAATTTCGTTCTACAACGACATCTGTGGCAAAGTGAACAGCTTCATGAATTTATACTGC
CAAAAGAATAAGGAGAATAAAAATTTATATAAACTGCAGAAACTGCATAAGCAAATTCTT
TGCATTGCAGACACCTCTTATGAAGTTCCTTATAAGTTTGAATCGGACGAGGAGGTATAT
CAGAGTGTGAACGGGTTCCTGGACAATATTTCATCCAAGCATATTGTTGAACGTTTACGC
AAAATTGGAGACAATTACAATGGGTATAACCTTGACAAAATTTACATCGTGTCGAAGTTT
TACGAATCGGTAAGCCAGAAGACCTATCGTGACTGGGAAACTATCAATACCGCCTTAGAA
ATTCATTACAACAATATTCTTCCTGGTAACGGCAAAAGCAAAGCCGATAAGGTAAAGAAG
GCTGTCAAGAACGACCTGCAAAAGTCTATCACAGAGATCAACGAGTTAGTCTCTAACTAC
AAATTATGTTCCGACGACAATATTAAAGCCGAAACCTACATCCATGAGATCTCACACATT
CTTAACAATTTTGAGGCCCAGGAGCTGAAATATAACCCAGAAATTCACCTTGTAGAGAGC
GAATTAAAAGCCTCCGAGCTGAAGAACGTTTTGGATGTAATCATGAACGCATTTCATTGG
TGCAGCGTATTTATGACAGAGGAGTTGGTCGACAAGGACAATAACTTTTACGCCGAGCTT
GAAGAAATCTACGATGAAATTTACCCGGTAATTAGTTTATATAATTTAGTTCGCAACTAC
GTAACTCAGAAACCCTACAGTACCAAGAAGATTAAATTGAACTTTGGGATCCCGACACTT
GCTGACGGTTGGAGTAAATCAAAAGAATACTCCAATAATGCAATTATCCTGATGCGCGAC
AATCTTTACTACTTGGGGATCTTTAACGCAAAGAACAAACCAGATAAGAAAATCATCGAG
GGCAACACCAGCGAGAATAAAGGCGATTACAAGAAAATGATCTATAATCTTTTGCCGGGA
CCGAACAAAATGATCCCAAAGGTTTTCCTGTCGTCGAAAACGGGAGTCGAGACATATAAA
CCATCTGCGTACATCTTGGAAGGTTACAAACAGAATAAGCATATTAAGTCTAGTAAAGAC
TTCGACATCACCTTTTGTCATGACCTGATTGATTATTTCAAGAACTGTATTGCTATCCAT
CCAGAATGGAAAAACTTCGGATTTGACTTCTCCGATACTAGCACCTACGAAGACATTTCG
GGTTTTTATCGCGAAGTAGAGCTTCAAGGGTACAAAATTGATTGGACATATATTAGCGAG
AAAGACATTGATTTGCTTCAAGAGAAGGGACAGTTATATTTATTCCAGATCTACAACAAA
GACTTCTCGAAGAAATCCACCGGTAATGATAATCTTCACACTATGTACCTGAAGAATTTA
TTTTCAGAGGAAAATCTGAAGGACATTGTACTTAAACTTAATGGAGAAGCCGAAATCTTC
TTCCGCAAGAGTTCCATTAAAAATCCGATTATTCATAAAAAGGGAAGTATCCTTGTGAAC
CGCACGTATGAGGCCGAAGAGAAGGATCAGTTTGGGAATATTCAAATTGTCCGCAAAAAC
ATCCCCGAGAACATCTACCAGGAACTGTATAAATACTTTAATGATAAATCTGATAAAGAG
TTATCAGACGAGGCTGCCAAACTGAAAAACGTAGTCGGTCATCATGAGGCAGCGACCAAT
ATTGTAAAGGACTACCGTTACACCTACGACAAGTATTTCCTTCACATGCCGATCACGATT
AATTTTAAGGCTAACAAGACCGGCTTTATCAATGACCGCATCTTGCAGTACATCGCGAAA
GAGAAAGATTTACACGTCATCGGAATTGATCGTGGAGAGCGTAATCTTATCTACGTCAGC
GTCATCGACACCTGTGGAAACATTGTGGAACAAAAAAGTTTTAATATCGTAAACGGCTAC
GACTATCAAATTAAACTTAAACAGCAAGAGGGAGCTCGCCAGATCGCTCGCAAAGAGTGG
AAAGAGATTGGGAAAATTAAAGAAATTAAAGAGGGTTACCTGTCGCTGGTAATTCACGAA
ATCTCGAAAATGGTCATCAAATATAATGCAATTATCGCTATGGAGGATCTGTCCTACGGG
TTCAAGAAGGGACGTTTTAAAGTAGAGCGCCAGGTGTATCAAAAATTCGAAACCATGTTG
ATCAATAAGCTTAACTATTTGGTCTTCAAAGATATTTCGATTACGGAGAACGGAGGTTTG
TTGAAAGGATATCAGCTGACGTATATCCCAGACAAGTTGAAAAACGTGGGGCATCAATGT
GGATGTATTTTCTATGTGCCCGCGGCCTACACGAGTAAGATCGATCCTACCACTGGTTTC
GTCAACATTTTCAAATTTAAAGATCTTACCGTGGATGCGAAGCGCGAATTTATTAAGAAA
TTTGATAGCATTCGCTATGATTCCGAAAAGAACCTGTTCTGTTTTACGTTCGACTATAAC
AATTTCATTACCCAAAACACGGTGATGAGCAAATCCTCTTGGTCAGTTTATACATACGGT
GTACGTATCAAACGCCGTTTCGTTAACGGACGCTTTTCCAATGAGTCTGATACAATCGAT
ATCACGAAAGATATGGAAAAAACATTAGAGATGACTGATATCAACTGGCGTGACGGGCAC
GACCTGCGTCAAGACATTATTGACTACGAGATTGTGCAGCATATCTTCGAAATCTTTCGC
TTAACTGTGCAAATGCGTAACTCGTTATCCGAGTTAGAAGACCGTGACTACGATCGCCTG
ATTTCACCCGTCTTGAACGAAAATAACATCTTCTACGATTCCGCGAAGGCTGGGGACGCA
TTGCCCAAGGACGCAGACGCGAATGGAGCGTACTGTATTGCGCTTAAAGGATTATATGAA
ATCAAGCAGATCACCGAAAATTGGAAGGAGGACGGGAAGTTCTCACGCGACAAACTGAAG
ATTTCAAATAAGGACTGGTTCGATTTCATTCAGAATAAGCGTTACCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 91
CCAGCGGCTAAAAAAAAGAAACTGGATGGCAGCGTGGATATGAATAATGGTACGAACAAC
TTTCAGAACTTCATCGGCATCTCCAGCCTTCAAAAGACTTTACGCAACGCATTGATTCCC
ACGGAGACTACGCAACAGTTTATCGTAAAAAATGGTATTATCAAAGAAGATGAATTACGC
GGGGAGAATCGCCAGATTCTTAAGGACATTATGGACGATTATTACCGTGGATTCATCAGT
GAGACACTGAGCTCCATTGATGACATCGACTGGACGTCATTGTTTGAAAAGATGGAAATC
CAGTTGAAAAATGGCGATAACAAAGATACATTGATTAAAGAGCAGACAGAGTACCGCAAA
GCAATTCACAAGAAATTCGCCAATGATGATCGTTTTAAGAACATGTTTAGTGCCAAGCTT
ATTTCGGATATCTTACCCGAATTCGTGATTCACAACAACAATTATTCGGCAAGTGAGAAA
GAGGAAAAGACCCAGGTTATCAAATTGTTTTCGCGCTTCGCCACTTCGTTCAAAGATTAT
TTCAAGAACCGTGCAAACTGTTTCTCCGCTGACGACATCAGTTCCAGCTCATGCCACCGT
ATTGTAAATGACAATGCGGAGATCTTTTTCAGTAATGCCTTAGTATATCGTCGCATTGTA
AAGAGCTTATCTAATGATGACATTAACAAGATCTCGGGTGATATGAAGGACTCACTTAAG
GAGATGAGTCTGGAAGAGATCTACTCCTACGAAAAATACGGGGAATTCATCACCCAGGAG
GGAATTTCATTCTACAACGATATCTGCGGCAAAGTTAACTCCTTTATGAATCTGTACTGT
CAAAAGAACAAGGAGAATAAAAACCTGTATAAATTGCAGAAACTTCATAAACAAATTTTG
TGTATCGCAGACACGAGTTATGAAGTACCTTATAAATTCGAATCCGACGAAGAGGTATAT
CAGTCCGTAAATGGGTTCCTGGACAATATCAGTAGTAAGCACATTGTGGAACGCTTACGC
AAAATTGGAGACAATTACAACGGGTATAACCTGGACAAAATCTACATCGTATCCAAATTT
TATGAAAGCGTGTCTCAAAAAACTTATCGTGATTGGGAAACAATCAACACGGCTCTTGAG
ATCCATTACAATAACATCTTGCCGGGTAACGGCAAATCGAAGGCAGACAAAGTTAAAAAA
GCAGTTAAGAACGACTTACAGAAAAGCATTACGGAGATTAACGAGTTAGTAAGTAATTAC
AAATTATGCTCCGACGATAATATCAAAGCTGAAACCTACATCCATGAAATTAGCCACATT
TTGAACAATTTCGAAGCGCAGGAGCTGAAATATAACCCTGAAATCCATCTGGTAGAGTCT
GAGTTGAAGGCGTCAGAACTGAAAAACGTTCTTGACGTCATCATGAATGCCTTTCACTGG
TGTAGTGTTTTTATGACTGAGGAGCTTGTAGATAAGGACAACAACTTCTATGCTGAACTT
GAAGAGATCTACGATGAAATCTACCCCGTAATCAGTCTGTATAATTTAGTTCGTAACTAC
GTCACGCAGAAACCCTATTCGACTAAGAAAATTAAGCTGAACTTTGGGATCCCTACTTTG
GCAGACGGGTGGAGCAAGAGTAAAGAATACAGTAATAATGCAATTATCTTGATGCGCGAT
AACTTATATTACTTAGGTATTTTCAATGCTAAGAACAAACCTGATAAGAAGATTATCGAA
GGAAATACGAGTGAGAATAAGGGAGACTACAAAAAGATGATTTACAACTTGCTGCCAGGG
CCTAATAAGATGATTCCAAAAGTTTTTCTGTCGAGCAAGACAGGGGTTGAAACTTATAAG
CCATCCGCTTATATCCTTGAGGGGTACAAGCAGAATAAGCATATCAAGTCCTCCAAAGAT
TTTGATATTACATTTTGCCACGACTTAATTGATTACTTCAAGAACTGCATCGCAATCCAT
CCCGAATGGAAGAATTTCGGCTTCGATTTCTCAGATACGTCCACGTATGAGGATATCTCA
GGCTTTTACCGCGAAGTTGAGCTGCAAGGTTATAAAATTGATTGGACATACATCTCCGAA
AAAGACATTGATCTTTTACAGGAAAAGGGCCAATTATACTTATTTCAAATCTATAACAAA
GATTTTAGCAAGAAGTCCACAGGTAATGATAACCTGCATACGATGTATTTGAAAAATCTT
TTCAGTGAAGAGAATTTGAAGGATATCGTCCTGAAGCTGAACGGTGAGGCTGAGATCTTC
TTCCGCAAATCGTCTATCAAAAACCCCATCATTCACAAAAAGGGAAGTATCTTAGTAAAC
CGCACTTATGAAGCGGAGGAAAAGGATCAGTTCGGGAACATCCAGATCGTGCGCAAGAAC
ATTCCAGAAAACATCTATCAGGAACTTTACAAATATTTCAATGACAAGTCTGATAAAGAA
TTATCAGACGAGGCGGCGAAACTTAAAAATGTTGTTGGACACCACGAAGCAGCGACGAAT
ATTGTAAAGGATTATCGCTACACATACGATAAATACTTTTTGCACATGCCAATCACCATT
AACTTTAAGGCGAACAAGACAGGTTTCATTAACGACCGTATTCTGCAATATATCGCAAAG
GAAAAAGACCTGCACGTTATTGGGATCGATCGTGGCGAACGCAATTTGATCTACGTAAGC
GTTATCGACACTTGCGGAAATATCGTTGAACAAAAAAGCTTTAATATCGTCAATGGATAC
GATTACCAAATCAAGCTGAAACAACAAGAAGGGGCACGTCAGATCGCTCGTAAAGAATGG
AAAGAGATTGGTAAGATCAAAGAGATTAAAGAAGGGTATCTTTCTTTAGTAATTCACGAG
ATTTCGAAAATGGTTATTAAATACAATGCGATTATTGCTATGGAAGACTTAAGCTACGGC
TTTAAGAAAGGTCGCTTCAAAGTGGAGCGCCAAGTGTATCAGAAGTTTGAAACGATGTTG
ATTAACAAATTAAATTACCTGGTCTTTAAGGACATCAGTATCACAGAAAATGGGGGGTTG
CTTAAAGGGTACCAGCTTACATACATCCCTGATAAACTGAAAAATGTCGGTCATCAGTGC
GGATGTATCTTCTATGTACCAGCAGCCTATACCAGTAAGATTGACCCTACTACTGGCTTT
GTGAATATTTTTAAATTCAAGGATTTAACCGTGGACGCCAAGCGTGAATTTATTAAAAAA
TTTGATTCGATTCGCTACGACAGTGAGAAAAACCTTTTCTGCTTTACCTTTGACTACAAC
AATTTTATTACCCAGAACACCGTAATGTCAAAGAGTTCGTGGTCTGTATATACCTACGGT
GTTCGCATCAAGCGCCGCTTCGTAAACGGGCGTTTCAGTAACGAATCTGACACCATCGAC
ATCACTAAAGATATGGAGAAGACATTGGAAATGACGGACATTAATTGGCGTGATGGCCAT
GACTTACGTCAGGACATTATTGATTACGAAATTGTGCAGCATATCTTCGAGATTTTCCGT
TTGACAGTTCAGATGCGCAACTCACTGAGTGAGTTAGAAGATCGCGATTACGACCGTCTG
ATCTCACCGGTCCTTAATGAAAACAACATTTTCTACGACTCAGCAAAGGCGGGTGATGCC
CTGCCAAAGGATGCGGACGCTAATGGCGCCTACTGCATCGCCCTGAAAGGATTGTATGAA
ATTAAGCAGATTACAGAAAATTGGAAGGAAGATGGTAAATTTAGCCGTGATAAATTAAAA
ATCTCGAACAAGGATTGGTTCGATTTTATTCAGAACAAACGTTATTTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 92
CCAGCGGCTAAAAAAAAGAAACTGGATGGCAGCGTGGATATGAACAATGGAACAAATAAT
TTTCAAAATTTTATCGGCATCTCAAGTCTTCAAAAAACCCTTCGCAATGCCCTGATTCCA
ACTGAAACAACCCAGCAATTTATCGTCAAGAACGGCATCATTAAGGAAGACGAGTTACGC
GGGGAGAACCGTCAAATCCTGAAAGATATCATGGATGACTACTATCGTGGGTTCATTTCG
GAAACCTTGTCTTCAATCGACGACATTGACTGGACGAGTCTTTTCGAGAAAATGGAAATT
CAGCTTAAAAATGGAGACAACAAGGATACTCTGATTAAGGAACAGACAGAATATCGCAAA
GCTATCCACAAAAAGTTCGCTAATGATGATCGTTTCAAAAATATGTTTTCTGCTAAATTG
ATTTCCGATATCTTGCCTGAATTTGTAATCCACAACAACAATTATTCTGCTTCCGAGAAG
GAAGAGAAGACCCAGGTCATTAAATTATTCAGCCGCTTTGCAACCAGCTTTAAAGACTAC
TTTAAGAATCGCGCTAACTGCTTTTCGGCGGATGACATCTCATCATCATCATGCCACCGC
ATTGTGAACGACAATGCGGAGATCTTCTTTTCGAATGCGTTAGTTTATCGTCGCATTGTC
AAAAGTCTTAGCAATGATGACATCAACAAGATCTCAGGAGACATGAAAGATTCCTTAAAG
GAGATGTCTCTTGAGGAAATCTATTCGTATGAGAAATACGGCGAGTTCATTACCCAGGAA
GGTATTAGTTTCTACAATGATATCTGCGGCAAAGTAAATTCTTTTATGAATCTGTATTGC
CAAAAAAACAAAGAAAACAAGAATCTTTATAAGTTACAAAAGTTACATAAGCAAATTCTG
TGCATCGCTGATACATCTTATGAGGTACCCTACAAATTTGAAAGTGATGAGGAGGTCTAT
CAGAGTGTCAACGGCTTCTTAGACAACATCTCTTCCAAACATATCGTGGAACGCCTGCGT
AAAATCGGAGATAACTACAACGGATATAACTTAGATAAAATCTACATCGTGTCCAAGTTT
TATGAAAGTGTGAGCCAAAAAACATATCGTGACTGGGAAACCATTAACACCGCATTGGAA
ATTCACTATAACAACATTTTGCCAGGCAACGGGAAAAGTAAGGCGGACAAAGTTAAGAAA
GCAGTTAAAAATGACCTGCAAAAAAGCATCACTGAAATTAACGAATTGGTATCGAATTAC
AAATTATGTAGCGACGATAATATCAAAGCAGAAACTTACATTCACGAGATTAGTCACATT
TTAAATAACTTCGAGGCCCAGGAATTGAAATACAATCCCGAAATTCATTTGGTTGAATCA
GAACTGAAAGCATCAGAGTTGAAAAATGTGTTAGATGTCATTATGAATGCGTTTCATTGG
TGCTCTGTGTTCATGACCGAGGAACTGGTTGATAAAGATAACAACTTTTACGCTGAATTG
GAGGAGATTTACGATGAGATTTACCCGGTCATTTCGCTTTATAACTTAGTGCGCAATTAT
GTGACGCAGAAACCATATTCCACGAAGAAAATCAAACTTAATTTTGGCATCCCTACTCTG
GCTGATGGTTGGTCGAAATCGAAAGAGTACAGCAACAACGCGATCATTCTTATGCGTGAC
AATCTTTACTATTTGGGCATTTTTAATGCCAAGAATAAGCCAGATAAGAAAATCATTGAG
GGGAATACTTCCGAGAATAAGGGGGATTACAAAAAGATGATCTATAACTTGCTGCCCGGC
CCCAACAAAATGATTCCTAAGGTTTTCTTGTCAAGCAAGACGGGCGTCGAAACATATAAG
CCGTCAGCTTATATTCTGGAAGGCTATAAACAGAATAAGCACATCAAGTCTTCCAAGGAC
TTTGACATCACTTTTTGCCACGATTTGATCGACTACTTTAAGAACTGTATTGCGATTCAT
CCGGAATGGAAGAACTTCGGTTTCGACTTTTCCGATACCTCAACATACGAGGATATCAGC
GGCTTCTACCGTGAAGTCGAGCTTCAAGGCTACAAGATCGATTGGACATATATTTCAGAG
AAGGACATTGATTTGTTACAAGAGAAAGGTCAACTTTACTTATTTCAGATCTATAACAAA
GACTTTTCGAAGAAATCGACAGGAAACGATAACTTACACACTATGTATTTAAAAAATCTG
TTTTCGGAGGAAAACCTGAAAGATATTGTGCTGAAACTTAACGGCGAGGCAGAGATCTTT
TTCCGTAAAAGCTCAATCAAGAATCCTATCATCCATAAAAAAGGTAGTATTCTTGTCAAC
CGCACATATGAAGCGGAGGAGAAGGACCAATTCGGAAACATCCAAATTGTCCGTAAGAAT
ATTCCGGAGAACATTTACCAAGAGTTGTATAAATACTTTAACGATAAGTCAGATAAGGAA
CTTAGCGATGAGGCGGCGAAGCTTAAAAACGTAGTTGGGCATCATGAAGCTGCTACCAAC
ATTGTAAAAGATTACCGTTACACCTATGACAAGTATTTCTTGCACATGCCCATTACGATC
AATTTCAAAGCAAATAAGACAGGCTTTATCAATGATCGCATCCTGCAGTACATTGCTAAA
GAGAAGGATTTGCATGTTATCGGTATTGATCGCGGAGAGCGCAATTTGATCTACGTCTCC
GTAATCGACACTTGCGGTAACATTGTTGAGCAGAAGTCGTTCAACATCGTTAATGGTTAT
GATTACCAAATCAAGCTGAAGCAGCAAGAGGGTGCCCGCCAGATCGCGCGTAAGGAATGG
AAAGAAATCGGGAAAATTAAAGAGATCAAAGAAGGCTATTTGTCTCTGGTAATTCACGAA
ATCAGCAAGATGGTGATCAAGTATAACGCGATCATTGCGATGGAGGATCTTTCTTATGGC
TTCAAGAAAGGGCGCTTTAAAGTCGAACGCCAGGTCTACCAGAAATTTGAGACAATGCTT
ATCAACAAGCTTAACTATCTTGTATTTAAGGATATTTCCATCACTGAGAACGGAGGACTT
TTAAAGGGGTACCAACTGACGTACATTCCTGATAAGCTGAAGAACGTTGGTCATCAATGC
GGATGCATCTTCTATGTGCCAGCGGCTTACACCTCCAAAATCGATCCCACTACAGGCTTT
GTCAATATCTTCAAATTCAAGGATTTGACCGTTGACGCGAAGCGCGAGTTTATCAAGAAG
TTTGATAGCATTCGCTACGACAGCGAAAAAAATTTATTTTGTTTTACTTTCGACTACAAT
AACTTTATTACTCAGAACACTGTCATGTCAAAGAGTTCGTGGAGTGTCTACACGTACGGA
GTACGTATTAAGCGCCGTTTCGTCAACGGACGCTTCTCAAACGAAAGCGACACGATCGAC
ATCACCAAAGACATGGAAAAAACTCTTGAGATGACGGATATCAATTGGCGCGACGGCCAT
GACCTGCGTCAGGATATCATTGATTACGAGATCGTTCAGCACATCTTCGAAATCTTCCGC
CTTACCGTCCAGATGCGCAACAGTTTAAGCGAGCTTGAAGACCGCGACTACGATCGTTTG
ATTAGCCCCGTTCTGAACGAGAATAATATTTTCTACGACAGCGCAAAGGCCGGTGATGCT
TTGCCAAAGGACGCAGACGCGAATGGAGCCTACTGCATCGCCCTGAAGGGCTTATATGAG
ATTAAGCAAATTACCGAAAATTGGAAGGAAGATGGTAAGTTCTCCCGTGATAAGCTTAAA
ATTAGCAATAAGGATTGGTTCGACTTCATCCAGAACAAACGTTACCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 93
CCAGCGGCTAAAAAAAAGAAACTGGATGGCAGCGTGGATATGAACAACGGAACAAACAAT
TTCCAAAACTTCATCGGTATCTCTTCGTTGCAGAAGACTCTGCGTAATGCTTTGATCCCG
ACGGAGACAACCCAACAATTTATCGTCAAAAACGGTATTATTAAGGAGGACGAGTTACGT
GGAGAAAATCGTCAAATCCTTAAGGACATCATGGACGATTATTATCGCGGGTTTATTTCT
GAAACCCTGAGCAGTATCGATGATATCGACTGGACCTCACTTTTTGAGAAAATGGAGATC
CAGTTGAAGAACGGTGATAACAAAGACACTCTGATCAAAGAGCAAACTGAATACCGCAAG
GCAATTCACAAAAAGTTCGCCAACGACGACCGTTTCAAGAATATGTTCTCAGCTAAGTTA
ATCAGCGACATTTTGCCAGAGTTCGTTATCCACAACAATAATTATAGTGCTTCAGAGAAG
GAGGAAAAAACCCAAGTGATTAAACTTTTTTCGCGCTTTGCAACCTCATTCAAGGACTAC
TTCAAGAATCGCGCGAATTGCTTCAGTGCGGACGACATTTCTTCTTCAAGTTGCCATCGT
ATCGTTAACGATAACGCGGAAATTTTCTTCTCTAATGCTTTGGTGTATCGCCGCATTGTA
AAATCGCTTAGTAACGATGACATTAATAAGATCTCAGGTGATATGAAAGATTCATTGAAG
GAAATGAGCTTGGAAGAGATTTACAGTTACGAAAAATATGGAGAATTTATTACTCAGGAA
GGCATCTCATTCTATAACGATATCTGCGGGAAGGTAAATTCGTTTATGAACTTATATTGC
CAGAAAAATAAAGAGAATAAAAATTTGTATAAGCTTCAGAAGTTGCACAAACAGATCCTG
TGCATTGCAGACACCTCGTATGAGGTTCCGTATAAATTTGAGTCCGATGAAGAAGTGTAT
CAGTCTGTGAATGGTTTCTTAGATAATATCTCTTCCAAGCATATTGTCGAACGCCTGCGC
AAAATTGGTGATAACTATAACGGATACAATCTGGATAAAATTTACATCGTTTCTAAATTT
TACGAGTCAGTCTCGCAGAAGACCTACCGCGACTGGGAAACAATTAACACGGCATTGGAG
ATTCACTACAATAATATCTTGCCTGGTAACGGTAAGTCTAAGGCAGATAAGGTAAAAAAA
GCTGTGAAAAACGACCTTCAGAAAAGCATCACGGAGATTAATGAGCTGGTGAGTAATTAC
AAATTATGTTCAGACGATAATATTAAAGCTGAAACGTATATCCATGAAATCTCGCATATC
TTGAACAACTTCGAGGCCCAAGAACTTAAATATAACCCCGAAATCCATTTAGTCGAGTCT
GAATTGAAAGCGTCGGAATTAAAAAACGTCTTAGACGTCATTATGAACGCGTTTCACTGG
TGTTCAGTTTTCATGACCGAAGAGCTGGTCGACAAAGACAACAACTTCTATGCGGAATTG
GAGGAAATCTATGATGAAATCTACCCTGTTATTTCACTGTATAACCTTGTGCGCAACTAT
GTCACTCAGAAGCCGTATTCGACCAAAAAAATTAAATTGAATTTCGGTATCCCTACTCTT
GCAGACGGATGGAGTAAAAGCAAGGAATACAGTAATAACGCCATTATTCTTATGCGCGAC
AATTTATACTACCTGGGCATCTTTAACGCAAAGAATAAGCCGGATAAGAAGATTATTGAG
GGTAACACCAGTGAGAACAAGGGCGACTATAAGAAGATGATCTATAACTTATTGCCAGGT
CCAAATAAAATGATCCCAAAAGTATTCTTATCATCAAAGACGGGAGTTGAAACCTATAAG
CCTAGTGCCTATATTCTTGAGGGATATAAACAGAACAAGCACATTAAGTCGTCTAAGGAT
TTTGACATTACGTTCTGCCATGACTTAATCGACTATTTTAAAAACTGTATTGCGATTCAC
CCCGAATGGAAGAATTTTGGATTCGATTTTTCGGATACCTCGACCTATGAAGATATTTCG
GGATTTTATCGTGAAGTGGAGTTGCAAGGCTATAAAATCGATTGGACCTATATCTCAGAA
AAAGACATTGATTTATTACAGGAAAAGGGACAACTGTACCTTTTCCAAATTTATAACAAG
GACTTTTCTAAAAAGTCCACAGGAAATGATAACCTTCACACCATGTACCTGAAGAACCTT
TTCTCAGAGGAAAACCTGAAGGACATTGTCCTTAAGTTAAATGGAGAAGCGGAGATCTTT
TTCCGTAAATCTAGTATCAAGAATCCGATTATCCATAAAAAAGGTTCGATTTTGGTAAAT
CGCACCTATGAAGCGGAAGAGAAAGATCAATTTGGTAACATCCAGATCGTGCGCAAGAAT
ATCCCGGAGAACATTTACCAAGAGCTGTATAAGTACTTCAATGATAAGTCTGATAAGGAA
CTGTCAGATGAAGCTGCGAAATTGAAGAACGTGGTTGGGCATCATGAAGCCGCTACCAAT
ATCGTCAAGGATTACCGTTATACCTATGACAAATATTTCTTACACATGCCGATTACGATC
AATTTTAAGGCAAACAAGACAGGATTCATCAACGACCGTATCTTGCAGTATATTGCCAAA
GAGAAGGATCTGCATGTGATCGGTATTGACCGCGGGGAGCGCAATTTAATCTATGTATCG
GTGATCGATACTTGTGGTAACATCGTAGAACAAAAGAGCTTTAACATCGTGAATGGTTAC
GACTATCAGATCAAGCTGAAACAACAGGAAGGAGCCCGCCAGATCGCTCGCAAGGAATGG
AAAGAAATCGGGAAAATTAAGGAAATCAAGGAAGGCTACCTTTCATTGGTCATTCACGAA
ATTTCGAAAATGGTAATTAAGTACAACGCGATCATCGCCATGGAGGACCTTTCGTACGGA
TTTAAGAAGGGTCGTTTCAAAGTTGAGCGCCAGGTATACCAAAAATTCGAGACTATGCTT
ATCAACAAACTTAACTACTTGGTCTTTAAGGACATTTCTATTACCGAAAACGGCGGCTTA
CTTAAAGGCTATCAATTGACATATATTCCCGACAAACTGAAGAATGTTGGACATCAATGC
GGGTGTATTTTCTATGTGCCGGCAGCTTACACTAGTAAGATCGACCCTACAACCGGGTTC
GTAAACATTTTTAAATTCAAAGACTTAACAGTCGATGCGAAGCGTGAATTTATTAAGAAG
TTTGATAGTATCCGCTATGACAGTGAAAAGAACTTGTTTTGCTTTACGTTCGACTACAAT
AACTTTATTACACAGAACACGGTCATGTCTAAATCATCATGGTCGGTTTACACATATGGG
GTGCGCATCAAGCGTCGCTTTGTAAATGGCCGTTTTAGTAATGAGAGCGACACAATCGAC
ATCACAAAGGATATGGAGAAAACTCTTGAGATGACAGACATCAATTGGCGTGACGGTCAT
GACTTACGCCAAGATATCATCGACTACGAAATCGTACAGCATATTTTTGAGATTTTTCGT
CTTACTGTGCAAATGCGTAATTCTTTATCCGAACTGGAAGATCGTGATTACGACCGCTTG
ATTAGTCCCGTCTTAAATGAGAACAATATTTTCTATGATTCTGCGAAAGCCGGAGATGCA
CTGCCCAAAGACGCTGATGCCAATGGCGCGTATTGCATTGCATTAAAAGGATTATATGAG
ATTAAACAGATTACCGAAAATTGGAAAGAGGACGGTAAATTCTCACGCGATAAATTGAAG
ATTTCTAACAAGGACTGGTTCGACTTTATCCAAAATAAACGTTATCTTAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 94
CCAGCGGCTAAAAAAAAGAAACTGGATGGCAGCGTGGATATGAATAACGGTACCAACAAC
TTTCAGAATTTCATTGGCATTAGCTCGCTTCAAAAAACTTTACGCAATGCTCTTATTCCG
ACTGAGACGACACAACAGTTTATCGTTAAGAATGGCATCATCAAAGAAGATGAATTACGC
GGAGAAAACCGCCAGATCCTGAAAGACATTATGGACGATTATTACCGTGGGTTCATCTCC
GAGACGTTGTCATCGATCGATGACATCGACTGGACGTCACTTTTTGAAAAAATGGAGATC
CAGTTAAAGAACGGTGACAATAAGGATACATTGATCAAAGAACAGACCGAGTACCGTAAA
GCGATTCATAAAAAGTTTGCGAACGATGATCGCTTCAAGAATATGTTTTCTGCGAAATTA
ATTTCCGACATTTTACCTGAATTTGTTATTCATAATAACAACTACTCGGCGTCTGAGAAA
GAGGAGAAAACCCAAGTGATTAAACTTTTTTCACGTTTCGCAACGTCGTTCAAAGACTAT
TTTAAAAATCGTGCTAATTGCTTTAGCGCGGATGACATCAGCTCTAGTTCATGTCATCGC
ATTGTCAACGATAATGCTGAGATCTTTTTCAGTAATGCGTTAGTGTACCGTCGTATTGTG
AAGTCCTTATCTAATGATGATATCAATAAGATCAGCGGGGATATGAAGGACTCACTTAAG
GAGATGAGCTTGGAGGAAATCTATTCCTATGAGAAGTATGGTGAGTTTATTACGCAAGAA
GGAATTAGCTTTTACAACGATATCTGTGGAAAGGTGAATTCGTTTATGAATTTGTATTGC
CAGAAAAATAAGGAGAACAAGAACCTTTATAAATTGCAAAAGTTACACAAGCAAATCCTG
TGCATTGCAGATACTTCCTACGAGGTGCCTTACAAGTTTGAATCCGACGAAGAGGTCTAC
CAATCTGTAAACGGTTTCTTAGATAATATTAGTTCCAAGCATATTGTGGAGCGCCTTCGT
AAAATTGGCGATAATTACAACGGTTACAATTTAGACAAAATTTACATTGTCAGTAAATTC
TACGAGTCCGTATCTCAAAAGACGTATCGTGATTGGGAGACTATCAATACGGCCCTGGAG
ATCCACTACAACAATATCTTGCCCGGTAATGGTAAGTCGAAGGCCGATAAAGTTAAGAAA
GCGGTGAAAAATGACTTACAGAAGTCAATCACCGAAATTAACGAATTGGTGTCCAATTAT
AAATTGTGTTCAGATGATAATATCAAAGCCGAGACCTACATTCATGAGATTTCCCATATC
TTAAATAATTTCGAGGCGCAAGAGCTTAAGTATAACCCAGAAATCCACCTGGTAGAATCT
GAGTTGAAGGCGTCAGAGTTAAAAAATGTTTTAGATGTCATTATGAACGCGTTTCACTGG
TGCTCCGTATTTATGACGGAGGAATTAGTAGATAAAGACAACAATTTCTATGCCGAACTT
GAGGAAATCTATGATGAGATCTATCCCGTCATTAGCCTGTATAACTTGGTCCGCAACTAT
GTTACCCAAAAACCGTACAGTACCAAGAAGATTAAGCTGAATTTCGGCATTCCTACACTG
GCTGATGGTTGGAGTAAATCGAAGGAATATTCGAATAACGCGATTATCTTGATGCGCGAC
AACTTATACTATTTGGGGATCTTTAACGCCAAAAACAAACCGGATAAGAAGATTATTGAG
GGAAACACATCAGAGAACAAAGGCGACTACAAAAAAATGATTTACAACTTGTTACCGGGG
CCTAACAAAATGATCCCGAAGGTGTTCTTATCCAGTAAAACAGGCGTTGAGACCTACAAA
CCTTCCGCATACATCCTGGAAGGGTATAAGCAGAACAAGCACATTAAGTCCAGCAAGGAT
TTCGATATTACCTTCTGTCATGATTTAATTGACTATTTCAAGAACTGTATTGCAATCCAC
CCCGAGTGGAAGAACTTCGGATTCGACTTCTCAGATACGAGCACATATGAGGACATCTCG
GGGTTCTATCGTGAAGTAGAACTGCAGGGATATAAAATTGATTGGACATATATTTCCGAA
AAAGACATCGACCTTTTACAAGAGAAGGGTCAACTTTACTTGTTCCAAATTTACAATAAA
GACTTCTCAAAAAAAAGCACGGGTAACGATAATTTACACACTATGTATTTAAAGAACCTT
TTCTCGGAAGAGAATTTAAAGGATATCGTATTGAAGTTGAATGGAGAAGCGGAGATCTTC
TTCCGTAAGTCCAGTATTAAAAACCCTATTATTCACAAGAAGGGATCGATTTTAGTTAAC
CGCACATACGAGGCCGAAGAGAAGGACCAATTTGGGAACATTCAAATTGTCCGCAAAAAC
ATCCCTGAGAACATTTATCAAGAGCTTTATAAGTACTTTAACGATAAGTCCGATAAGGAA
TTGTCAGATGAGGCGGCAAAGTTGAAGAATGTCGTGGGGCATCATGAAGCTGCCACCAAC
ATTGTGAAGGACTACCGCTACACTTACGACAAATACTTCCTGCACATGCCCATTACGATC
AATTTTAAGGCCAATAAGACAGGCTTTATTAACGACCGTATTCTTCAATATATCGCTAAG
GAGAAGGACCTTCATGTGATTGGGATCGACCGCGGAGAACGTAATTTAATTTATGTGTCC
GTCATCGATACGTGTGGAAATATCGTGGAACAGAAATCATTCAATATCGTGAATGGCTAT
GATTACCAGATCAAATTAAAACAGCAGGAGGGCGCTCGCCAAATTGCGCGTAAGGAATGG
AAAGAGATCGGAAAAATCAAAGAAATCAAAGAAGGATATTTGTCATTGGTGATCCATGAG
ATTTCAAAAATGGTAATTAAATATAATGCAATTATCGCAATGGAAGACCTGTCCTATGGT
TTTAAGAAGGGTCGTTTCAAGGTAGAACGCCAAGTGTATCAAAAGTTCGAGACGATGCTG
ATCAATAAGCTGAATTATCTTGTGTTTAAGGACATTAGCATCACGGAAAATGGAGGGCTG
TTGAAAGGCTATCAACTGACGTATATCCCTGACAAGCTGAAAAATGTTGGCCATCAGTGC
GGGTGCATTTTCTACGTCCCCGCGGCGTATACAAGCAAGATCGATCCTACTACGGGATTC
GTAAATATTTTTAAATTCAAAGACTTAACCGTGGACGCCAAGCGCGAATTCATTAAGAAG
TTTGATAGCATTCGCTACGATTCAGAAAAAAATCTTTTCTGTTTTACGTTCGATTACAAC
AATTTTATCACCCAGAACACAGTGATGAGCAAGTCATCCTGGTCTGTCTATACCTACGGT
GTCCGTATCAAACGCCGCTTCGTCAACGGACGCTTCTCTAATGAATCTGATACCATTGAC
ATCACCAAGGACATGGAAAAGACACTTGAGATGACAGATATTAACTGGCGTGACGGACAT
GACCTGCGTCAGGACATCATCGATTATGAGATTGTTCAGCATATCTTCGAGATCTTCCGC
CTGACAGTACAAATGCGCAATTCACTGTCAGAACTTGAAGACCGCGACTATGACCGCCTG
ATCTCTCCAGTATTAAATGAGAACAATATCTTTTATGACAGTGCTAAGGCCGGCGATGCC
CTTCCGAAAGATGCTGATGCTAACGGAGCTTATTGTATTGCATTAAAGGGTCTTTATGAG
ATCAAGCAAATTACCGAGAATTGGAAGGAGGATGGCAAATTCTCGCGCGACAAACTGAAA
ATCAGTAACAAGGACTGGTTCGATTTTATTCAGAATAAACGTTACCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 95
CCAGCGGCTAAAAAAAAGAAACTGGATGGCAGCGTGGATATGAATAACGGAACGAACAAC
TTCCAGAACTTCATCGGCATCAGTTCTTTACAAAAAACCCTGCGTAACGCCCTTATTCCG
ACTGAGACAACACAACAGTTCATCGTTAAAAACGGAATTATCAAAGAGGACGAGTTGCGC
GGCGAGAATCGCCAAATTTTGAAAGATATTATGGACGACTATTATCGTGGTTTTATTTCA
GAAACACTGAGTTCGATTGACGATATCGATTGGACGAGCCTGTTTGAGAAAATGGAAATC
CAGTTGAAAAATGGCGATAATAAAGACACTTTAATCAAAGAACAAACCGAGTATCGTAAA
GCGATCCATAAAAAGTTCGCTAATGACGATCGTTTTAAGAATATGTTCAGTGCGAAACTG
ATTTCAGACATTTTGCCCGAGTTCGTGATCCATAATAACAACTATTCCGCCTCGGAAAAG
GAAGAAAAAACCCAGGTGATTAAGCTGTTCAGTCGCTTCGCAACATCTTTCAAGGATTAT
TTCAAGAATCGCGCGAATTGCTTCAGTGCGGACGATATTTCTAGTTCAAGCTGCCATCGT
ATCGTTAATGATAACGCGGAGATTTTTTTTAGCAATGCTCTGGTGTACCGCCGCATTGTT
AAGTCACTGTCCAACGATGATATTAACAAGATCTCAGGAGACATGAAAGACTCGCTTAAA
GAGATGAGTCTGGAAGAGATCTATTCTTATGAGAAGTATGGCGAGTTTATTACCCAAGAA
GGAATCTCATTCTACAATGATATTTGTGGAAAGGTGAACAGCTTTATGAATCTTTACTGC
CAAAAAAACAAGGAGAATAAGAATCTTTACAAACTTCAGAAGTTACATAAACAGATTTTG
TGTATTGCGGATACGTCTTATGAAGTCCCCTACAAATTTGAATCGGATGAAGAGGTATAC
CAAAGTGTGAACGGATTCTTGGACAATATTTCTTCTAAACATATTGTTGAACGCTTACGT
AAGATCGGGGATAACTACAATGGCTACAATCTTGACAAAATCTACATTGTTAGCAAATTC
TACGAGAGTGTCAGCCAAAAGACGTACCGCGATTGGGAAACAATTAATACTGCGCTTGAG
ATTCACTATAATAACATTTTACCAGGCAACGGCAAGTCCAAGGCGGATAAAGTTAAAAAA
GCTGTTAAAAACGATTTGCAAAAATCTATCACAGAAATTAACGAGTTAGTTAGTAACTAC
AAACTGTGCTCCGATGACAACATTAAGGCTGAGACGTATATCCATGAGATCTCTCACATC
TTAAACAATTTTGAAGCTCAAGAACTTAAGTACAATCCGGAAATCCACCTGGTGGAATCC
GAGCTGAAGGCTAGCGAACTGAAGAACGTATTGGACGTGATCATGAACGCGTTCCACTGG
TGTTCTGTCTTTATGACGGAAGAGCTTGTCGACAAAGATAATAACTTTTACGCGGAACTT
GAGGAAATTTACGATGAGATTTACCCAGTTATTTCATTGTATAACCTTGTCCGTAATTAC
GTGACCCAAAAGCCTTATAGTACGAAAAAAATCAAATTAAATTTTGGAATCCCAACACTG
GCTGACGGTTGGAGCAAATCTAAGGAGTATTCTAATAACGCAATCATCTTAATGCGTGAC
AACCTGTATTATTTGGGTATCTTCAATGCCAAAAATAAGCCTGACAAAAAGATTATCGAA
GGAAATACTTCGGAGAATAAGGGGGATTACAAAAAAATGATTTACAATTTGCTGCCCGGG
CCGAACAAGATGATCCCCAAAGTGTTCTTATCCTCGAAGACTGGTGTAGAAACATACAAG
CCAAGCGCATACATTCTGGAGGGTTACAAGCAAAACAAACACATCAAATCTTCAAAAGAC
TTTGACATTACATTTTGCCATGATCTTATTGACTACTTCAAAAACTGCATTGCTATTCAC
CCCGAGTGGAAGAACTTTGGGTTTGACTTCAGCGACACGTCTACGTATGAGGACATCTCC
GGGTTCTACCGTGAAGTTGAGTTACAAGGGTATAAGATTGACTGGACGTATATTTCAGAG
AAAGATATCGATCTTTTGCAGGAAAAGGGCCAGTTATATTTATTCCAGATTTACAACAAG
GACTTTAGTAAGAAGTCAACAGGAAATGACAACTTGCATACGATGTATTTGAAAAATCTT
TTTTCTGAGGAAAATCTTAAGGACATCGTACTGAAATTGAATGGCGAGGCTGAAATCTTC
TTCCGTAAATCCTCCATTAAGAATCCCATTATCCACAAAAAGGGGTCTATCCTGGTGAAT
CGTACCTACGAGGCAGAGGAGAAGGATCAATTCGGAAATATTCAGATTGTTCGTAAGAAC
ATCCCCGAGAACATTTATCAAGAATTGTATAAGTACTTTAATGACAAATCTGACAAAGAG
TTATCCGACGAAGCTGCGAAACTGAAAAACGTTGTTGGTCACCACGAGGCCGCCACTAAT
ATCGTAAAAGACTACCGTTATACCTATGACAAGTACTTTTTGCACATGCCGATCACTATC
AACTTCAAGGCGAATAAGACGGGCTTCATTAACGATCGTATCCTGCAATACATCGCCAAG
GAGAAGGACCTTCACGTCATTGGGATTGACCGTGGTGAGCGTAACCTGATTTATGTAAGC
GTCATTGATACCTGCGGTAATATCGTCGAACAGAAAAGTTTCAACATTGTAAATGGATAT
GACTATCAGATCAAACTTAAGCAGCAGGAGGGTGCACGCCAGATTGCCCGCAAGGAATGG
AAGGAGATTGGGAAGATTAAGGAAATTAAAGAAGGTTACTTATCACTGGTTATTCACGAG
ATCAGTAAAATGGTAATCAAATATAACGCGATCATTGCCATGGAGGATCTGAGCTATGGC
TTTAAAAAGGGCCGTTTCAAAGTCGAGCGCCAGGTATATCAAAAGTTTGAAACAATGCTG
ATTAACAAATTAAACTATCTGGTTTTCAAAGATATTTCGATCACTGAAAATGGCGGGCTG
TTGAAGGGATACCAACTTACATACATCCCTGACAAACTGAAAAATGTCGGTCACCAATGT
GGATGTATCTTTTATGTACCAGCAGCGTATACGAGCAAAATCGATCCAACTACGGGTTTT
GTGAACATCTTTAAGTTCAAGGATTTGACAGTAGATGCCAAACGCGAGTTCATTAAAAAA
TTTGATTCAATTCGCTACGATTCAGAGAAAAATCTTTTTTGTTTCACGTTCGATTACAAT
AATTTCATTACGCAGAACACAGTAATGTCAAAGTCAAGCTGGTCGGTCTACACGTATGGA
GTCCGTATTAAACGTCGTTTTGTAAACGGCCGTTTCTCAAATGAATCAGATACAATTGAT
ATTACGAAGGATATGGAGAAGACATTAGAGATGACTGACATTAACTGGCGCGACGGACAT
GATCTTCGTCAGGACATTATTGATTATGAGATTGTACAGCATATCTTTGAGATCTTCCGC
CTGACCGTTCAGATGCGCAATTCGTTGTCCGAGTTAGAAGACCGCGATTACGACCGTTTA
ATCAGTCCCGTCTTAAACGAAAATAACATCTTCTACGATTCAGCCAAGGCAGGCGATGCC
TTGCCAAAGGATGCTGACGCAAATGGCGCATACTGTATTGCGTTGAAAGGCCTTTATGAA
ATCAAGCAAATTACCGAAAACTGGAAAGAAGACGGAAAATTCTCCCGTGATAAGTTGAAA
ATCTCTAATAAGGATTGGTTCGATTTCATCCAAAATAAACGCTATTTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 96
CCAGCGGCTAAAAAAAAGAAACTGGATGGCAGCGTGGATATGAACAACGGAACTAATAAT
TTCCAAAATTTTATAGGCATCTCTTCTTTACAGAAGACTCTTCGTAACGCCCTAATCCCG
ACTGAGACCACACAACAATTCATAGTGAAAAATGGGATCATTAAAGAAGACGAGCTGCGT
GGGGAGAACAGGCAGATCCTAAAAGACATAATGGACGATTATTATAGAGGGTTCATCTCA
GAGACATTATCTAGCATCGACGACATTGACTGGACCTCCCTGTTTGAAAAAATGGAAATC
CAGCTGAAGAATGGTGACAATAAAGACACATTAATAAAAGAACAAACAGAGTACAGGAAA
GCCATCCACAAGAAGTTCGCAAACGATGACAGATTCAAAAATATGTTCAGTGCGAAGCTA
ATATCCGACATCTTACCAGAGTTTGTAATACACAATAACAATTACAGCGCGAGCGAAAAG
GAAGAGAAAACGCAAGTAATTAAGCTTTTTAGTAGGTTCGCTACCTCTTTCAAAGATTAC
TTCAAAAATCGTGCTAACTGCTTCTCAGCCGACGACATATCTTCAAGTTCCTGTCACCGT
ATCGTGAATGATAACGCTGAGATATTCTTCTCAAACGCCCTTGTATACCGTAGGATCGTA
AAGTCCTTATCTAACGATGATATAAACAAGATCAGTGGAGACATGAAAGACAGCCTTAAA
GAGATGTCTCTAGAAGAAATTTACTCCTATGAAAAGTATGGGGAGTTTATAACACAGGAG
GGGATCAGCTTCTACAACGACATCTGCGGAAAGGTGAACAGTTTCATGAATCTTTACTGC
CAGAAGAATAAAGAGAACAAAAATCTTTATAAGCTTCAAAAGTTGCACAAACAAATACTG
TGCATTGCCGATACATCATATGAGGTCCCCTATAAGTTCGAATCTGATGAGGAAGTTTAT
CAATCTGTTAACGGCTTTCTAGACAATATCAGCTCAAAACACATCGTAGAAAGACTGAGG
AAAATAGGTGATAATTATAATGGATACAACTTGGATAAAATATATATAGTCTCTAAATTT
TACGAGTCAGTATCCCAGAAAACGTATAGGGATTGGGAGACCATCAACACGGCGTTAGAG
ATTCATTACAATAACATCTTACCGGGAAACGGAAAAAGTAAGGCGGACAAAGTAAAGAAA
GCCGTTAAAAATGACTTACAAAAGAGTATAACAGAAATAAACGAACTAGTAAGCAACTAC
AAGCTTTGTTCCGATGATAATATCAAGGCCGAGACATATATCCATGAGATCTCCCACATT
CTAAACAATTTCGAAGCGCAAGAACTTAAATATAATCCCGAAATCCACCTGGTGGAAAGT
GAACTAAAGGCTAGTGAGTTAAAGAACGTTCTTGATGTTATCATGAACGCCTTCCATTGG
TGCTCTGTTTTTATGACCGAGGAGTTGGTTGATAAAGATAATAATTTCTACGCTGAATTA
GAGGAGATATACGACGAAATCTACCCAGTGATTTCACTATACAACTTGGTCAGGAACTAT
GTTACACAAAAGCCGTACAGCACTAAGAAAATTAAGCTAAATTTCGGTATCCCCACGTTA
GCCGACGGGTGGAGCAAGTCCAAAGAATATTCCAACAATGCGATTATTTTAATGCGTGAC
AATCTTTATTACCTTGGCATCTTCAATGCCAAAAACAAACCTGACAAAAAGATTATAGAA
GGTAATACGTCCGAGAACAAAGGCGATTACAAGAAGATGATTTATAACCTACTGCCCGGA
CCAAACAAAATGATCCCCAAAGTTTTTCTTAGTTCTAAAACCGGCGTAGAGACGTATAAA
CCTTCTGCCTATATCTTAGAGGGATATAAGCAGAACAAACATATCAAATCTTCCAAGGAC
TTTGATATTACATTCTGCCACGATTTAATTGACTACTTCAAAAATTGCATAGCGATACAT
CCGGAGTGGAAGAACTTTGGCTTCGACTTCAGTGATACATCCACCTATGAGGATATATCA
GGCTTCTATCGTGAGGTCGAATTGCAAGGGTACAAAATCGATTGGACGTATATATCCGAG
AAAGACATAGACCTTCTTCAAGAAAAGGGGCAGTTATATTTATTCCAAATATACAACAAG
GACTTCAGTAAGAAGTCAACAGGTAATGACAACTTACACACCATGTACTTGAAAAATTTA
TTTTCTGAAGAAAACCTAAAGGACATTGTACTAAAACTGAACGGGGAGGCAGAAATTTTT
TTTAGAAAGAGCAGCATAAAAAACCCAATAATTCATAAGAAAGGAAGCATTTTAGTTAAT
AGGACGTACGAGGCAGAGGAAAAGGACCAGTTTGGCAATATCCAGATCGTAAGGAAAAAT
ATTCCTGAAAACATATATCAGGAACTATATAAATACTTTAACGACAAATCCGACAAAGAA
TTATCCGACGAGGCTGCAAAGCTGAAGAACGTCGTAGGGCACCATGAGGCAGCGACTAAT
ATTGTGAAAGACTATAGGTATACATACGACAAATACTTTCTGCACATGCCCATCACGATT
AACTTCAAGGCGAACAAGACGGGATTCATTAACGACCGTATATTACAATATATTGCTAAG
GAGAAAGATCTGCATGTAATAGGTATCGACAGAGGCGAACGTAATTTAATCTACGTGTCC
GTCATCGACACGTGCGGGAACATCGTAGAGCAAAAGAGTTTTAATATAGTAAATGGCTAT
GATTACCAAATTAAGCTAAAGCAGCAAGAAGGAGCAAGACAGATAGCTAGGAAAGAATGG
AAGGAGATAGGAAAAATAAAGGAGATCAAGGAGGGGTATCTTAGCCTAGTAATTCATGAA
ATATCTAAGATGGTTATCAAATACAACGCTATCATAGCGATGGAAGACTTATCTTATGGT
TTCAAGAAAGGAAGGTTCAAAGTAGAGCGTCAAGTTTATCAAAAGTTCGAAACGATGTTG
ATTAATAAACTAAACTATTTGGTATTTAAAGATATATCTATCACCGAGAATGGTGGTCTA
CTAAAGGGTTACCAGCTTACATACATACCGGACAAACTTAAAAACGTCGGACATCAGTGT
GGATGCATTTTCTACGTTCCAGCTGCATATACCAGCAAGATCGACCCAACGACTGGGTTC
GTAAATATTTTTAAATTCAAGGATTTGACTGTCGACGCCAAAAGAGAGTTCATAAAAAAG
TTCGATTCAATTAGGTACGACAGCGAAAAGAATTTGTTCTGCTTTACTTTTGACTATAAC
AATTTCATTACTCAGAACACTGTAATGTCTAAGTCCTCTTGGTCAGTCTATACTTATGGC
GTTCGTATCAAACGTAGATTTGTTAACGGTAGATTCTCAAATGAAAGTGATACAATAGAT
ATCACGAAAGATATGGAGAAAACATTAGAAATGACAGACATAAACTGGAGAGACGGACAT
GACTTGAGACAGGACATTATTGACTACGAGATCGTGCAGCACATCTTTGAGATCTTTCGT
TTGACCGTACAAATGCGTAACAGTTTATCTGAGCTTGAGGACAGGGACTACGATAGATTG
ATATCACCTGTATTAAATGAGAATAACATCTTCTATGATTCCGCAAAAGCAGGCGACGCT
CTACCCAAAGACGCTGATGCGAACGGTGCTTATTGCATAGCTTTAAAGGGTTTGTATGAG
ATCAAACAGATAACAGAAAATTGGAAGGAAGATGGTAAGTTCTCCCGTGACAAGCTTAAA
ATATCAAATAAGGACTGGTTCGATTTTATACAGAATAAGCGTTATTAAAACGTCCGGCAG
CGACCAAAAAAGCCGGCCAGGCGAAGAAAAAAAAAGCGTCAGGTAGCGGCGCAGGCAGCC
CGAAAAAGAAACGTAAAGTCGAGGATCCGAAAAAGAAACGTAAGGTTATTCCGGGCTAA
SEQ ID NO: 97
CCAGCGGCTAAAAAAAAGAAACTGGATGGCAGCGTGGATATGAACAATGGAACTAATAAC
TTCCAGAATTTCATTGGTATCTCCTCTTTACAAAAAACTCTAAGAAACGCCCTAATTCCG
ACTGAAACTACACAGCAATTCATCGTCAAAAACGGGATCATTAAGGAGGATGAGTTGAGG
GGTGAAAATCGTCAAATTCTTAAAGACATCATGGACGACTACTACAGGGGGTTCATCAGC
GAGACGTTATCTAGTATAGACGATATAGACTGGACTTCACTGTTCGAGAAGATGGAAATC
CAATTAAAAAATGGGGACAATAAAGATACACTTATAAAGGAACAGACAGAGTATAGAAAG
GCAATACACAAAAAGTTTGCCAACGACGATCGTTTCAAGAACATGTTTAGTGCTAAATTG
ATTTCAGATATTCTGCCGGAATTTGTTATTCACAACAATAATTATAGCGCCAGTGAGAAA
GAAGAAAAAACGCAGGTTATCAAACTGTTCAGTCGTTTCGCTACATCTTTTAAGGATTAC
TTTAAAAACCGTGCAAATTGTTTTTCAGCCGACGATATTAGTAGCAGCTCTTGTCACCGT
ATTGTTAATGATAATGCGGAGATTTTCTTTTCAAACGCATTGGTCTACAGGAGGATAGTC
AAGTCCCTTTCAAATGACGACATTAATAAGATCTCAGGTGACATGAAAGATTCCTTAAAG
GAAATGTCCCTGGAAGAGATCTATTCCTATGAAAAGTACGGTGAGTTCATTACTCAAGAG
GGTATAAGCTTTTACAATGACATATGTGGTAAGGTTAATAGCTTTATGAACCTGTATTGC
CAGAAGAACAAAGAAAATAAGAATCTGTATAAGTTGCAAAAGCTACACAAACAAATTTTG
TGCATTGCCGATACATCATACGAGGTGCCATACAAATTCGAGAGCGATGAGGAGGTTTAT
CAGAGCGTGAATGGATTCCTGGACAATATTAGTAGTAAGCATATCGTGGAAAGGCTTAGA
AAGATAGGTGACAATTACAATGGCTACAATCTGGATAAAATCTACATCGTCTCAAAATTC
TATGAAAGTGTATCCCAGAAGACGTACCGTGATTGGGAAACTATCAACACCGCTCTGGAG
ATACATTACAACAATATACTTCCCGGAAACGGCAAGTCAAAAGCCGACAAAGTCAAAAAA
GCGGTCAAGAACGATTTACAAAAGTCTATCACTGAAATTAATGAATTAGTTAGTAATTAC
AAACTGTGTAGTGATGATAATATTAAGGCAGAGACTTACATACACGAAATTTCACACATT
TTAAACAACTTCGAGGCACAGGAACTTAAATATAATCCTGAAATTCACCTGGTTGAAAGT
GAATTGAAAGCCAGCGAGCTAAAGAACGTTTTGGACGTAATCATGAACGCATTCCACTGG
TGCTCTGTCTTTATGACAGAGGAACTAGTGGATAAGGACAATAATTTTTATGCGGAGCTG
GAGGAAATATACGATGAGATATATCCCGTAATATCATTATATAATCTGGTAAGAAACTAT
GTGACTCAAAAGCCGTATAGCACCAAGAAAATTAAACTTAATTTCGGCATACCCACTTTA
GCGGACGGCTGGTCAAAATCCAAAGAGTATAGTAATAATGCCATCATCCTGATGCGTGAC
AACCTGTACTATTTAGGTATATTTAACGCCAAAAATAAACCCGACAAAAAGATTATAGAG
GGCAACACCTCAGAGAACAAAGGTGATTATAAGAAGATGATTTACAACCTTTTACCCGGT
CCTAATAAGATGATTCCCAAAGTCTTTCTATCTAGCAAAACTGGTGTTGAAACATACAAA
CCCTCAGCTTATATTTTAGAAGGGTATAAGCAGAATAAGCATATTAAAAGCTCCAAAGAT
TTCGATATTACCTTTTGCCATGACTTGATAGACTATTTCAAAAATTGTATTGCCATTCAC
CCTGAATGGAAAAACTTCGGATTTGACTTCTCTGACACATCCACCTACGAAGACATTTCA
GGTTTTTACAGGGAAGTCGAGCTACAGGGTTATAAAATTGATTGGACATACATCAGCGAG
AAAGATATTGACCTACTTCAAGAAAAAGGGCAGCTATACCTGTTCCAGATATACAATAAA
GACTTCAGTAAAAAAAGCACCGGGAACGATAATCTTCACACAATGTACTTAAAAAATTTA
TTTAGTGAAGAGAATCTGAAGGATATAGTGCTGAAGTTAAACGGGGAGGCAGAGATATTT
TTTAGAAAATCTAGTATTAAGAATCCGATCATCCACAAGAAGGGTTCTATCCTTGTTAAT
AGGACTTATGAGGCAGAAGAAAAAGACCAATTCGGCAACATACAAATTGTCCGTAAAAAT
ATCCCTGAGAACATTTATCAGGAACTATACAAGTACTTCAATGATAAAAGCGACAAGGAG
CTGAGCGACGAGGCTGCTAAGTTAAAGAATGTGGTGGGCCACCATGAGGCAGCAACGAAT
ATTGTGAAGGACTATCGTTATACCTACGATAAATACTTTCTTCATATGCCGATCACCATT
AATTTCAAGGCAAACAAAACTGGCTTCATTAACGATCGTATCTTACAATATATCGCAAAA
GAGAAAGACCTTCACGTTATCGGGATCGATAGAGGCGAGCGTAACCTAATTTATGTTTCT
GTGATAGACACCTGTGGGAACATAGTCGAACAGAAATCATTTAATATTGTTAACGGCTAC
GATTATCAGATAAAGTTGAAGCAACAAGAGGGTGCACGTCAAATAGCAAGGAAAGAATGG
AAAGAAATAGGCAAGATTAAAGAAATAAAAGAAGGTTATTTATCCCTTGTAATACACGAA
ATTAGCAAAATGGTGATTAAATATAATGCGATCATTGCCATGGAGGATCTTTCTTACGGC
TTCAAAAAGGGGAGATTCAAAGTCGAGAGGCAGGTGTATCAGAAGTTTGAGACCATGCTA
ATCAATAAACTAAATTATCTAGTATTCAAAGACATAAGCATCACCGAAAATGGCGGCTTG
TTGAAGGGTTATCAATTGACCTACATCCCAGATAAACTAAAAAACGTAGGGCATCAATGC
GGATGTATATTTTACGTTCCAGCCGCATACACTTCCAAAATCGATCCAACTACGGGTTTT
GTGAACATCTTCAAATTCAAAGACTTGACTGTCGATGCTAAGAGGGAGTTTATCAAGAAA
TTTGACTCCATTAGATACGACAGTGAGAAGAATCTGTTCTGTTTTACCTTTGATTATAAC
AACTTTATAACTCAAAACACAGTCATGAGTAAGTCATCTTGGTCAGTGTATACGTATGGT
GTGAGGATTAAAAGGAGGTTTGTTAACGGGAGATTTTCCAATGAAAGTGATACAATAGAT
ATAACCAAGGACATGGAAAAGACTCTTGAAATGACCGACATTAACTGGAGAGATGGCCAC
GACTTACGTCAAGATATAATCGATTACGAGATAGTGCAACATATCTTTGAGATATTTAGG
CTTACTGTCCAAATGCGTAACTCATTAAGTGAGTTGGAGGACAGGGATTACGATAGGCTA
ATAAGTCCTGTTCTTAACGAAAACAATATATTCTACGATTCAGCAAAGGCGGGAGACGCC
CTGCCCAAGGACGCGGATGCTAACGGCGCATACTGTATTGCCCTGAAAGGCTTGTACGAG
ATAAAACAGATCACGGAGAACTGGAAAGAAGATGGAAAATTCAGTCGTGACAAGTTAAAA
ATTAGTAACAAAGACTGGTTCGACTTTATTCAGAACAAGAGATATCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 98
CCAGCGGCTAAAAAAAAGAAACTGGATGGCAGCGTGGATATGAACAACGGAACCAATAAC
TTTCAAAACTTTATAGGCATCTCCAGTCTACAGAAGACACTACGTAACGCTTTGATACCA
ACTGAGACCACGCAGCAGTTTATCGTCAAGAACGGTATTATAAAGGAAGACGAGCTAAGG
GGGGAAAACCGTCAGATCTTAAAGGACATCATGGATGACTACTACAGAGGCTTCATAAGT
GAGACTTTGTCTAGTATAGACGACATCGACTGGACCAGTTTATTTGAGAAGATGGAAATT
CAGTTAAAGAACGGGGACAATAAAGACACACTAATTAAAGAGCAGACCGAATACAGAAAA
GCTATACACAAAAAGTTTGCCAACGATGATAGATTCAAAAATATGTTTTCAGCAAAATTG
ATTTCCGACATATTGCCAGAATTCGTAATCCATAATAACAATTATTCTGCAAGTGAGAAG
GAAGAGAAGACCCAAGTAATCAAGCTGTTTTCCCGTTTTGCTACGAGTTTCAAAGATTAT
TTCAAGAATAGGGCTAATTGTTTCTCCGCGGACGACATAAGTAGCAGTTCCTGTCACAGG
ATTGTGAACGATAATGCTGAGATATTTTTTTCCAATGCCCTAGTGTATAGGAGAATAGTT
AAAAGCTTAAGCAACGACGATATCAATAAAATTTCAGGGGACATGAAGGACAGCTTAAAG
GAAATGAGTTTGGAGGAGATTTACAGTTATGAAAAATACGGAGAGTTTATAACTCAGGAA
GGCATCTCTTTCTATAATGATATCTGTGGGAAGGTAAACTCCTTCATGAATTTATATTGC
CAGAAGAATAAGGAAAACAAAAATCTTTACAAGCTTCAAAAGTTACATAAGCAGATCTTA
TGTATTGCCGACACGAGTTATGAAGTGCCTTATAAATTCGAGAGTGATGAGGAAGTGTAT
CAGTCTGTTAACGGATTCCTAGATAATATAAGTTCCAAACATATAGTCGAGAGGCTGAGG
AAGATTGGCGATAACTATAATGGATATAATCTTGACAAAATCTATATAGTCTCTAAATTT
TATGAAAGCGTCAGCCAGAAGACATATAGAGATTGGGAAACTATAAACACAGCCCTTGAA
ATACATTACAATAACATCCTACCCGGCAATGGTAAGTCTAAGGCAGACAAAGTTAAAAAA
GCAGTAAAGAATGACTTACAGAAGTCAATCACGGAGATAAATGAGTTGGTCAGTAACTAC
AAATTATGCTCCGACGATAATATTAAGGCCGAAACATATATACACGAGATAAGTCATATA
TTAAACAATTTCGAAGCCCAGGAGTTAAAATATAACCCTGAAATTCATCTGGTCGAAAGT
GAGTTAAAGGCCAGTGAGTTAAAGAATGTACTTGACGTAATTATGAATGCTTTTCATTGG
TGCTCCGTGTTCATGACCGAGGAGTTAGTAGATAAAGACAATAACTTTTACGCCGAACTT
GAAGAGATATACGACGAGATTTATCCGGTAATCAGCTTGTACAACTTAGTTAGAAATTAT
GTAACACAGAAGCCTTACTCTACTAAAAAAATAAAACTGAACTTTGGTATCCCAACTCTT
GCAGATGGTTGGAGTAAAAGCAAGGAATATAGCAACAATGCGATCATCTTGATGAGAGAC
AACTTGTACTATTTGGGAATCTTCAACGCGAAAAATAAACCCGACAAAAAAATCATCGAA
GGGAATACCTCTGAGAATAAAGGTGACTATAAGAAAATGATTTACAATCTACTTCCTGGT
CCTAATAAAATGATCCCGAAAGTGTTTCTTAGTTCTAAGACTGGTGTCGAGACGTACAAA
CCTAGCGCGTACATCTTAGAAGGGTACAAGCAGAATAAACACATCAAATCAAGCAAAGAC
TTCGATATTACTTTTTGCCATGACTTGATAGACTACTTTAAAAACTGCATAGCAATCCAC
CCGGAGTGGAAAAACTTTGGCTTTGATTTCTCTGACACCTCTACATATGAGGACATATCT
GGTTTTTACCGTGAGGTTGAATTGCAGGGATACAAAATTGACTGGACTTACATATCTGAA
AAAGATATCGATCTATTGCAGGAGAAAGGCCAGCTTTACCTTTTCCAGATCTATAATAAG
GACTTCTCTAAGAAGTCTACAGGGAATGATAATTTGCACACTATGTACTTAAAAAATCTG
TTTTCCGAGGAAAACTTGAAAGACATTGTTTTAAAGTTGAACGGAGAAGCTGAAATATTT
TTCAGAAAGAGCTCCATAAAAAACCCGATCATTCATAAGAAGGGATCTATCCTGGTTAAC
AGAACGTACGAAGCGGAAGAAAAAGACCAATTCGGAAACATTCAAATTGTTAGAAAGAAT
ATCCCTGAGAACATCTACCAGGAGTTATATAAGTATTTTAATGATAAGTCAGATAAGGAA
CTATCTGACGAAGCGGCGAAGCTTAAAAATGTTGTAGGACACCATGAGGCTGCTACAAAT
ATAGTCAAGGACTACCGTTATACCTACGATAAGTACTTTCTACACATGCCCATTACCATC
AATTTTAAAGCTAATAAAACGGGTTTTATCAACGATCGTATCCTACAATATATTGCGAAA
GAGAAGGATTTGCATGTCATTGGCATTGATAGAGGTGAGAGGAACCTAATATACGTATCC
GTGATTGATACGTGCGGGAACATAGTTGAACAGAAATCATTTAATATAGTTAATGGGTAC
GACTATCAGATTAAGCTAAAGCAACAAGAAGGCGCCAGGCAAATTGCCCGTAAAGAATGG
AAAGAGATCGGGAAGATCAAGGAAATAAAAGAAGGATACCTTTCCCTGGTCATCCATGAA
ATTAGCAAAATGGTGATTAAGTACAATGCCATAATCGCGATGGAGGACTTAAGCTACGGG
TTCAAAAAGGGGAGGTTTAAGGTGGAGAGGCAAGTGTACCAGAAATTTGAGACCATGCTA
ATCAACAAACTGAACTACCTAGTTTTTAAGGACATTTCAATTACAGAGAATGGAGGACTT
TTAAAGGGTTACCAACTAACGTATATACCAGATAAGTTGAAAAATGTCGGTCACCAGTGT
GGCTGCATCTTTTACGTTCCCGCCGCTTATACATCTAAAATTGATCCAACCACAGGCTTT
GTAAATATCTTTAAATTCAAAGATTTAACTGTGGATGCAAAAAGAGAGTTTATCAAGAAA
TTCGATAGCATTCGTTATGATAGCGAGAAGAACCTGTTCTGCTTTACTTTCGACTATAAC
AACTTTATAACTCAAAACACCGTGATGTCAAAAAGCTCATGGTCAGTCTACACCTATGGT
GTAAGGATTAAAAGGCGTTTCGTGAATGGGAGATTCTCCAATGAAAGTGACACGATCGAC
ATAACAAAGGACATGGAGAAGACACTAGAGATGACTGATATTAATTGGAGAGACGGACAC
GATCTGCGTCAAGATATAATTGATTATGAGATAGTACAGCACATATTTGAGATCTTCCGT
TTGACTGTCCAAATGCGTAATTCCCTTTCTGAGCTGGAAGATAGGGACTATGATAGATTA
ATATCCCCTGTACTAAATGAGAACAACATTTTCTATGATAGTGCAAAAGCCGGGGATGCA
TTGCCGAAAGACGCTGACGCTAATGGGGCGTACTGTATAGCTTTAAAGGGGCTTTACGAA
ATAAAGCAGATAACCGAAAACTGGAAGGAAGATGGCAAATTCTCAAGGGACAAACTTAAG
ATCTCTAACAAGGATTGGTTCGATTTTATACAAAACAAACGTTATTTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 99
CCAGCGGCTAAAAAAAAGAAACTGGATGGCAGCGTGGATATGAATAATGGTACAAACAAC
TTTCAGAATTTCATTGGGATCTCTAGCTTACAGAAGACCCTGAGGAATGCGTTGATTCCA
ACTGAAACAACCCAGCAATTCATCGTGAAAAATGGGATAATCAAAGAGGATGAGTTAAGG
GGTGAAAACCGTCAAATATTGAAGGATATTATGGACGACTACTACCGTGGATTCATCTCA
GAGACGTTGAGCAGCATTGACGACATAGACTGGACTAGCCTTTTCGAGAAGATGGAAATT
CAGTTAAAGAACGGAGATAACAAAGATACACTAATCAAGGAACAGACAGAATACAGAAAA
GCAATTCATAAGAAATTCGCTAATGACGATCGTTTTAAAAACATGTTCTCTGCAAAATTA
ATTAGCGACATTCTGCCGGAATTCGTTATACATAATAATAACTACAGTGCTTCTGAAAAG
GAAGAGAAAACTCAGGTAATAAAACTGTTCTCTCGTTTTGCCACATCCTTCAAAGACTAC
TTTAAAAATAGAGCGAACTGCTTTAGCGCCGACGATATTAGTTCTTCCTCATGCCACAGG
ATTGTCAACGATAATGCAGAGATATTCTTTTCTAACGCACTAGTCTACAGAAGGATTGTA
AAGTCTTTGTCAAATGATGACATAAACAAGATTAGTGGAGATATGAAAGACTCTCTAAAG
GAAATGAGCCTTGAGGAGATATACTCTTATGAAAAGTACGGTGAGTTTATTACCCAAGAA
GGCATTAGTTTCTATAATGACATTTGTGGAAAAGTTAACAGTTTTATGAATCTATACTGT
CAAAAAAATAAGGAGAATAAAAATCTTTATAAGTTGCAAAAACTGCATAAGCAGATATTA
TGTATAGCAGACACGAGCTATGAGGTACCGTACAAGTTCGAGAGCGATGAGGAAGTCTAC
CAATCTGTCAACGGATTTTTGGACAACATTTCTTCAAAACATATTGTGGAGAGGCTTAGG
AAAATAGGCGACAATTATAATGGATATAACTTAGATAAGATATATATTGTTTCCAAATTC
TACGAATCTGTAAGCCAGAAGACATACAGAGATTGGGAAACGATAAACACAGCCCTTGAA
ATTCACTATAACAACATACTACCTGGAAACGGCAAATCAAAGGCCGACAAAGTTAAGAAG
GCCGTAAAGAATGATTTACAGAAGAGCATAACGGAGATCAATGAGCTGGTGTCTAACTAT
AAATTGTGTAGCGATGACAACATAAAAGCCGAGACTTACATTCACGAAATTTCACACATA
CTTAACAACTTTGAAGCTCAGGAATTAAAGTATAATCCCGAAATACACCTTGTGGAGTCC
GAACTAAAGGCTAGTGAGCTTAAGAACGTCCTAGACGTAATTATGAATGCCTTCCACTGG
TGTAGTGTTTTTATGACCGAGGAACTTGTTGACAAAGATAATAATTTTTATGCAGAACTA
GAAGAGATATACGATGAAATATACCCGGTGATCAGTTTGTACAATCTTGTCAGGAACTAT
GTGACACAAAAGCCCTATTCAACAAAGAAAATAAAACTTAATTTCGGAATTCCTACGTTA
GCTGATGGCTGGTCTAAATCCAAGGAATACAGCAACAACGCTATAATTCTGATGAGAGAT
AACTTGTACTATCTAGGCATCTTCAATGCCAAAAATAAGCCTGATAAGAAGATTATAGAG
GGCAACACTTCAGAGAACAAGGGCGACTACAAGAAAATGATCTATAACCTATTGCCTGGC
CCAAACAAGATGATTCCGAAGGTCTTCCTATCATCCAAGACCGGCGTTGAGACATACAAG
CCATCAGCGTATATTTTAGAGGGGTACAAACAAAACAAGCACATAAAGTCTAGTAAAGAC
TTCGATATAACATTTTGTCATGACTTAATTGACTACTTTAAGAATTGCATCGCTATACAC
CCGGAATGGAAGAATTTCGGCTTCGACTTCTCTGATACATCTACCTACGAGGACATTAGC
GGGTTTTACCGTGAAGTCGAATTACAAGGGTATAAGATAGATTGGACGTACATCTCTGAG
AAAGACATAGACTTGCTTCAGGAAAAGGGCCAGTTGTATCTATTCCAAATATACAATAAG
GATTTTTCCAAGAAATCTACGGGTAATGACAATCTTCACACAATGTATCTTAAGAACCTT
TTCTCAGAAGAGAACCTGAAGGACATTGTCTTAAAACTAAATGGCGAAGCTGAGATTTTT
TTCAGGAAGTCTTCAATTAAGAACCCGATAATCCACAAGAAGGGGAGTATTCTTGTGAAT
AGAACTTACGAGGCCGAAGAAAAAGACCAATTTGGTAACATCCAGATAGTCAGAAAGAAC
ATTCCAGAGAACATCTACCAAGAGCTATACAAATATTTCAACGACAAGTCCGATAAGGAA
CTGTCCGATGAGGCAGCCAAGTTGAAGAATGTCGTGGGTCATCATGAAGCTGCTACTAAC
ATTGTCAAGGACTATCGTTATACTTACGACAAGTATTTCCTACACATGCCGATAACAATT
AATTTCAAGGCTAACAAAACAGGCTTTATCAACGATCGTATCTTGCAGTACATAGCTAAG
GAAAAGGATTTGCATGTGATTGGCATTGATAGAGGGGAGCGTAACTTGATATATGTGTCT
GTCATAGACACGTGTGGCAACATCGTCGAACAGAAATCATTCAACATAGTAAACGGCTAC
GATTACCAAATTAAGCTGAAACAGCAAGAGGGTGCACGTCAAATTGCGCGTAAAGAGTGG
AAAGAAATTGGTAAAATCAAGGAAATTAAAGAAGGCTACTTGTCTCTTGTTATACATGAA
ATTTCCAAGATGGTTATAAAGTATAACGCGATAATTGCTATGGAAGACTTATCATACGGG
TTTAAAAAGGGGAGGTTCAAGGTAGAGAGGCAGGTCTATCAAAAGTTCGAGACGATGTTG
ATTAATAAACTAAACTATCTAGTGTTCAAAGATATCAGCATTACGGAGAACGGGGGGCTA
CTGAAAGGATATCAACTAACGTACATTCCCGATAAGTTAAAGAACGTTGGTCATCAATGT
GGTTGCATCTTCTACGTGCCTGCTGCCTATACGTCCAAAATAGATCCAACTACTGGATTT
GTTAACATCTTTAAATTCAAAGATTTAACCGTAGACGCCAAAAGGGAATTTATAAAAAAA
TTTGACAGCATCCGTTACGATAGCGAAAAGAATCTGTTCTGTTTTACTTTCGACTACAAT
AATTTCATCACGCAAAATACGGTAATGTCTAAGTCAAGTTGGAGCGTCTACACGTATGGA
GTCAGGATCAAGAGGCGTTTCGTAAATGGAAGATTCTCTAATGAGTCAGATACTATAGAC
ATCACGAAAGATATGGAGAAAACCTTGGAGATGACGGATATTAACTGGCGTGATGGACAC
GATTTAAGACAGGACATTATTGACTATGAGATTGTGCAACACATCTTCGAAATATTCCGT
CTAACAGTCCAAATGAGGAATAGCCTAAGTGAATTGGAGGACCGTGATTACGATAGGCTT
ATAAGTCCTGTCCTTAACGAAAACAATATTTTCTATGATAGTGCTAAGGCGGGGGACGCA
CTGCCTAAAGACGCAGATGCTAACGGGGCATACTGCATTGCGTTAAAGGGTCTGTACGAA
ATCAAGCAGATTACGGAAAACTGGAAAGAGGATGGCAAGTTTAGCAGAGATAAGTTGAAG
ATAAGTAACAAAGATTGGTTTGACTTTATTCAGAATAAAAGGTATTTAAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 100
CCAGCGGCTAAAAAAAAGAAACTGGATGGCAGCGTGGATATGAATAACGGCACTAATAAT
TTCCAGAATTTCATCGGCATTAGCAGCTTACAAAAGACGTTGAGGAATGCCTTAATACCC
ACAGAAACTACTCAACAATTTATAGTGAAGAATGGGATAATTAAGGAAGACGAGTTGAGA
GGTGAAAATAGGCAAATCTTGAAAGACATTATGGATGACTACTACAGGGGCTTCATTAGT
GAAACGTTGTCTTCAATAGATGACATTGATTGGACTTCTTTGTTTGAGAAGATGGAAATA
CAGTTAAAGAACGGCGACAATAAGGATACACTTATCAAAGAGCAAACAGAATATAGAAAA
GCAATTCACAAAAAGTTTGCTAACGATGATAGGTTCAAGAACATGTTTAGCGCTAAACTA
ATATCAGACATCCTTCCCGAGTTCGTTATTCATAACAATAACTATAGTGCAAGTGAAAAA
GAGGAGAAGACACAGGTGATTAAGCTGTTCTCCAGATTCGCGACTTCTTTCAAAGATTAC
TTCAAAAACAGAGCCAACTGTTTTTCAGCTGACGATATCTCTAGTAGTAGTTGTCACCGT
ATAGTGAACGATAACGCTGAGATCTTCTTTAGCAATGCATTAGTGTATAGAAGGATAGTT
AAGTCTCTAAGCAATGATGATATCAATAAAATTTCCGGAGACATGAAGGACTCCCTAAAG
GAAATGTCCTTAGAAGAGATCTACTCATATGAGAAATACGGGGAATTTATTACGCAGGAA
GGGATCTCCTTTTACAATGACATATGCGGGAAGGTCAACTCTTTCATGAACTTATACTGC
CAAAAGAACAAGGAGAACAAGAATTTATATAAACTTCAGAAACTTCACAAACAAATACTG
TGCATAGCCGATACCTCATATGAGGTTCCTTACAAATTTGAATCAGATGAAGAGGTATAC
CAATCCGTTAACGGCTTTCTTGACAATATTAGCTCAAAGCACATCGTGGAGAGGTTGAGA
AAGATTGGTGATAATTATAATGGCTACAATCTAGATAAGATATATATTGTTAGCAAGTTC
TACGAGTCTGTGTCCCAAAAAACATATAGGGATTGGGAGACAATTAATACTGCTCTAGAA
ATCCATTACAACAACATCCTTCCTGGAAATGGCAAGAGTAAGGCCGACAAAGTCAAGAAA
GCAGTGAAAAATGATCTGCAAAAATCAATTACTGAGATAAACGAGCTAGTATCTAATTAC
AAGCTTTGTAGCGACGATAACATTAAGGCAGAAACGTACATACACGAGATTAGTCACATC
TTAAATAATTTTGAAGCCCAAGAACTGAAATATAACCCTGAGATACACCTTGTTGAATCC
GAGTTAAAGGCGTCTGAACTAAAAAACGTGTTAGACGTTATTATGAATGCCTTCCACTGG
TGTAGCGTCTTTATGACTGAGGAGTTGGTTGATAAGGATAATAACTTTTACGCTGAATTG
GAAGAAATTTATGACGAAATCTATCCTGTTATTTCTCTATATAATTTGGTGAGAAATTAC
GTAACGCAAAAGCCCTATAGTACGAAAAAAATAAAACTAAATTTCGGGATCCCTACCCTA
GCCGACGGTTGGTCTAAATCCAAGGAGTACTCAAACAATGCAATAATATTGATGAGGGAC
AACCTGTACTACCTAGGCATATTTAATGCCAAAAATAAGCCCGATAAAAAGATTATAGAA
GGGAACACGTCAGAAAATAAAGGAGACTATAAGAAAATGATCTACAACCTTTTGCCCGGC
CCCAATAAAATGATCCCGAAGGTCTTCCTAAGTAGCAAGACTGGCGTAGAGACCTACAAA
CCATCTGCATACATTTTGGAGGGGTACAAGCAAAACAAGCACATAAAGAGTAGTAAGGAT
TTTGACATTACATTCTGCCATGACTTAATTGACTACTTTAAAAATTGCATCGCAATTCAC
CCTGAATGGAAAAATTTTGGATTTGATTTCTCTGATACTTCAACATATGAGGATATTTCA
GGGTTCTACAGGGAGGTCGAACTACAGGGTTACAAAATAGACTGGACGTATATTTCTGAG
AAAGATATAGATTTGCTTCAGGAAAAGGGTCAGCTATATCTGTTCCAGATATATAATAAG
GACTTCTCCAAAAAGAGTACCGGAAATGATAATCTGCACACAATGTACTTAAAAAACTTG
TTCTCTGAGGAGAATCTAAAAGACATCGTACTAAAACTTAACGGGGAGGCCGAAATTTTT
TTTAGGAAGTCCAGCATCAAGAACCCGATTATTCATAAAAAAGGTAGCATTTTGGTGAAC
CGTACTTATGAGGCGGAAGAAAAAGACCAATTCGGTAATATTCAAATCGTTAGAAAGAAC
ATCCCTGAGAACATTTATCAGGAACTATACAAATACTTTAACGACAAATCAGATAAGGAG
CTTTCTGATGAGGCAGCTAAATTGAAAAATGTAGTGGGACATCACGAAGCAGCCACTAAC
ATAGTGAAGGACTACAGATACACATACGATAAGTACTTCCTGCACATGCCTATTACAATT
AACTTTAAAGCAAATAAAACAGGGTTTATTAACGACAGAATCTTACAGTATATTGCCAAA
GAAAAGGATCTGCATGTGATAGGAATAGACAGAGGAGAAAGAAACCTGATATACGTCTCC
GTGATTGATACATGTGGGAACATAGTAGAACAGAAGTCCTTTAACATTGTTAATGGGTAC
GATTATCAAATTAAATTAAAACAACAAGAAGGAGCACGTCAAATAGCTAGGAAAGAATGG
AAAGAGATAGGAAAAATTAAGGAAATTAAGGAGGGTTACCTGTCCCTTGTAATTCATGAA
ATATCCAAAATGGTAATTAAATATAACGCGATCATCGCGATGGAAGATCTAAGCTACGGG
TTCAAAAAAGGCAGGTTTAAGGTGGAGAGGCAAGTTTACCAAAAGTTCGAGACAATGTTG
ATTAATAAGTTAAACTACTTAGTTTTCAAAGATATCTCCATAACCGAGAATGGCGGGCTT
TTAAAAGGGTACCAACTAACATATATCCCGGATAAATTGAAGAACGTTGGACACCAGTGT
GGCTGCATATTTTATGTACCCGCTGCGTATACTTCTAAAATTGACCCGACCACCGGGTTT
GTAAACATATTCAAGTTTAAGGACCTAACAGTTGACGCCAAACGTGAGTTCATCAAGAAG
TTCGATAGTATAAGGTATGACTCTGAGAAGAACCTTTTCTGCTTCACGTTTGACTATAAT
AATTTCATCACCCAAAATACAGTTATGTCAAAAAGCTCTTGGTCAGTATATACGTATGGC
GTAAGGATTAAGCGTAGGTTCGTGAACGGTAGATTTTCCAACGAGTCAGATACTATTGAT
ATTACCAAGGATATGGAGAAGACATTAGAAATGACAGATATAAATTGGAGGGATGGGCAC
GATCTAAGGCAAGATATCATTGATTACGAAATTGTTCAGCACATATTCGAGATATTCCGT
CTTACAGTACAAATGCGTAACAGCTTGTCTGAGTTGGAAGATCGTGACTATGACAGGTTG
ATATCACCGGTCTTGAACGAGAACAATATATTCTACGACAGCGCTAAGGCGGGAGACGCT
CTGCCTAAAGACGCAGATGCCAATGGGGCGTACTGCATTGCCTTAAAAGGCTTATACGAG
ATTAAACAGATCACAGAGAACTGGAAAGAGGACGGCAAGTTTTCTAGAGATAAATTGAAA
ATCTCAAACAAAGACTGGTTCGATTTCATCCAAAACAAAAGATACCTTAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 101
CCAGCGGCTAAAAAAAAGAAACTGGATGGCAGCGTGGATATGAACAATGGAACTAACAAC
TTCCAGAACTTTATCGGCATCTCTTCCCTCCAAAAGACACTGAGAAATGCACTGATCCCA
ACCGAAACGACTCAACAATTTATTGTTAAGAACGGCATCATAAAAGAAGACGAGCTTCGC
GGCGAGAACCGCCAGATACTTAAGGATATTATGGACGATTATTACCGAGGCTTTATCAGC
GAAACTCTTAGCTCTATTGATGATATCGACTGGACCTCCCTCTTCGAAAAAATGGAGATA
CAGCTCAAGAACGGCGATAATAAAGACACCTTGATAAAGGAACAGACTGAGTACAGGAAA
GCGATCCACAAGAAATTCGCGAACGACGACAGGTTTAAAAACATGTTCTCTGCAAAATTG
ATATCCGACATCTTGCCGGAATTTGTGATACACAACAATAACTATAGCGCTTCAGAGAAA
GAAGAGAAGACCCAAGTAATCAAGTTGTTCAGCCGCTTCGCAACGTCTTTTAAAGATTAC
TTTAAGAACCGGGCCAATTGTTTCTCCGCGGATGATATTAGCTCATCAAGTTGCCATCGA
ATTGTCAATGATAATGCGGAGATCTTCTTCAGCAATGCGCTGGTCTACAGACGAATCGTA
AAAAGTCTTTCAAATGACGACATCAATAAGATTAGTGGAGATATGAAGGATTCCCTTAAG
GAAATGAGTCTTGAAGAAATATACTCATACGAAAAGTACGGGGAATTTATTACCCAGGAG
GGGATCTCCTTCTATAACGACATCTGTGGAAAAGTAAACTCATTCATGAACCTGTACTGT
CAGAAAAACAAAGAAAACAAAAATCTGTATAAACTCCAAAAATTGCACAAGCAAATATTG
TGTATAGCGGACACATCATACGAGGTTCCATATAAGTTCGAAAGTGATGAAGAAGTCTAC
CAATCAGTGAATGGGTTTCTGGACAACATTAGTTCCAAGCACATAGTTGAACGACTGCGA
AAGATTGGTGACAATTACAACGGCTATAATTTGGACAAGATTTATATAGTTAGCAAATTT
TATGAATCCGTATCACAAAAGACTTATAGAGACTGGGAAACAATCAACACGGCACTTGAG
ATCCATTATAACAATATTCTTCCAGGGAACGGCAAAAGCAAGGCTGATAAGGTAAAAAAG
GCCGTTAAGAATGATCTTCAAAAATCCATAACGGAGATCAACGAACTTGTAAGTAACTAC
AAATTGTGCTCTGACGACAATATAAAGGCTGAAACGTATATTCACGAGATTAGCCATATC
CTGAATAACTTTGAGGCCCAAGAACTCAAGTATAACCCGGAAATACATTTGGTAGAAAGC
GAGCTTAAAGCGAGTGAGCTGAAAAACGTCCTCGATGTGATCATGAATGCTTTCCACTGG
TGTAGTGTCTTTATGACTGAGGAGTTGGTTGATAAAGACAATAATTTCTACGCTGAACTG
GAAGAAATTTACGACGAAATCTATCCAGTGATCTCCCTCTATAACCTCGTTCGAAACTAC
GTGACGCAGAAACCTTATTCTACAAAGAAAATTAAGTTGAACTTCGGCATTCCTACACTT
GCTGACGGATGGTCCAAATCCAAAGAGTACTCAAACAACGCAATCATCCTCATGCGGGAT
AACCTTTATTATTTGGGCATTTTCAACGCCAAAAACAAACCTGATAAAAAGATAATTGAA
GGCAATACGAGTGAGAACAAGGGCGACTACAAAAAAATGATATATAACTTGTTGCCAGGC
CCCAACAAGATGATTCCTAAAGTTTTTCTGTCTTCTAAGACTGGAGTTGAAACTTACAAA
CCCTCCGCCTACATTCTTGAAGGGTATAAACAGAATAAGCACATAAAGTCCTCAAAGGAT
TTCGACATTACGTTTTGCCATGACCTCATCGACTATTTCAAGAACTGTATCGCCATACAT
CCGGAGTGGAAGAATTTTGGATTTGATTTCTCCGACACATCTACCTATGAAGACATAAGC
GGTTTCTACCGGGAGGTCGAGCTTCAGGGCTATAAGATAGATTGGACATACATTAGTGAA
AAAGATATCGATCTTCTGCAAGAAAAGGGACAACTTTACCTTTTTCAGATTTATAATAAA
GACTTTTCAAAAAAGTCCACAGGGAACGATAATCTGCACACCATGTATCTCAAGAATCTG
TTTAGTGAAGAAAACCTTAAAGACATAGTTTTGAAGCTTAACGGAGAGGCTGAGATTTTT
TTTAGAAAGTCCTCAATTAAAAACCCTATAATACACAAGAAAGGCTCTATTCTTGTTAAC
AGGACATATGAAGCCGAGGAGAAAGATCAGTTTGGCAATATCCAGATTGTTCGCAAGAAT
ATCCCGGAAAATATATATCAGGAGCTGTATAAATACTTTAACGACAAGAGCGACAAGGAG
CTGAGTGACGAGGCCGCGAAGCTTAAGAATGTAGTAGGTCACCACGAAGCAGCCACCAAT
ATCGTCAAAGACTATAGGTACACGTACGACAAGTACTTTTTGCACATGCCTATAACTATA
AACTTCAAAGCTAATAAAACTGGGTTTATTAATGACAGGATTCTCCAATACATCGCTAAA
GAGAAGGATCTGCATGTAATTGGCATAGACAGAGGTGAGAGAAACTTGATATATGTCAGC
GTAATAGACACATGTGGCAATATCGTGGAACAGAAGTCTTTTAACATCGTCAATGGTTAC
GACTACCAAATTAAGTTGAAACAGCAGGAAGGCGCACGACAGATCGCACGAAAGGAATGG
AAAGAGATAGGCAAAATAAAAGAAATAAAGGAGGGCTATCTCAGTCTCGTTATACACGAA
ATTTCAAAAATGGTTATTAAGTACAATGCAATCATAGCGATGGAGGATCTCAGTTATGGG
TTCAAAAAGGGTCGGTTTAAAGTTGAGCGCCAAGTGTACCAAAAGTTCGAGACAATGCTG
ATTAACAAGCTGAACTACCTCGTCTTCAAAGATATAAGTATTACGGAGAACGGTGGCCTT
CTTAAAGGCTATCAACTTACTTACATCCCGGACAAGCTCAAAAACGTAGGGCACCAATGC
GGGTGTATTTTCTATGTGCCTGCGGCATATACGTCAAAGATTGACCCAACCACAGGATTC
GTAAACATATTCAAGTTTAAGGACCTCACCGTTGATGCGAAAAGGGAGTTCATTAAAAAA
TTTGATTCTATTCGATATGATAGTGAGAAAAATCTCTTTTGTTTCACATTTGACTATAAT
AATTTTATTACTCAGAATACTGTCATGAGCAAGTCATCTTGGTCAGTGTACACATACGGG
GTGCGGATCAAACGCAGGTTCGTCAATGGTCGCTTCTCAAACGAATCAGACACCATTGAC
ATCACAAAGGACATGGAAAAAACCCTTGAGATGACCGACATTAATTGGCGCGATGGTCAT
GATCTGCGGCAAGACATCATAGACTACGAAATCGTCCAACACATCTTTGAGATCTTTCGC
TTGACGGTCCAAATGCGGAACTCCCTGTCCGAGCTCGAGGATAGAGATTATGATCGGCTG
ATATCTCCCGTGCTTAATGAAAATAACATCTTCTACGACTCCGCCAAGGCGGGTGATGCC
CTGCCGAAGGATGCGGATGCTAATGGCGCTTATTGCATTGCTCTTAAGGGGCTCTATGAG
ATAAAGCAGATCACGGAAAACTGGAAAGAAGACGGTAAGTTTAGTAGAGACAAGCTGAAG
ATCTCAAATAAAGACTGGTTTGATTTCATACAGAACAAGCGGTACCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 102
CCAGCGGCTAAAAAAAAGAAACTGGATGGCAGCGTGGATATGAACAATGGCACTAACAAT
TTTCAGAATTTCATCGGCATTTCAAGTCTGCAAAAAACTCTGAGGAATGCTTTGATCCCT
ACTGAAACCACTCAGCAATTTATAGTCAAGAACGGTATAATTAAAGAAGATGAACTCAGG
GGTGAAAATAGACAAATACTCAAGGACATTATGGATGACTATTATAGAGGCTTCATCTCA
GAGACTCTCTCATCAATAGATGATATCGATTGGACTAGCCTTTTCGAGAAAATGGAGATT
CAGTTGAAAAATGGTGATAACAAAGATACGTTGATAAAGGAACAGACCGAGTACAGGAAA
GCCATTCATAAGAAATTTGCTAATGACGATAGATTTAAGAATATGTTTAGTGCAAAACTG
ATTAGTGACATTCTGCCGGAGTTCGTTATCCATAATAATAACTACTCTGCATCCGAAAAG
GAGGAAAAGACGCAAGTTATTAAACTGTTCAGCCGCTTCGCCACAAGCTTCAAGGACTAC
TTCAAAAATAGAGCCAACTGCTTTTCTGCCGACGATATATCATCATCTTCATGCCATCGG
ATCGTTAACGATAACGCCGAGATATTCTTCAGCAACGCCCTTGTATATCGAAGAATAGTC
AAAAGTCTGAGTAATGATGATATTAATAAAATTAGCGGTGATATGAAAGACTCCCTGAAG
GAAATGTCACTGGAGGAAATTTATAGTTACGAAAAGTACGGCGAATTCATTACTCAAGAA
GGCATATCCTTCTATAACGACATTTGCGGAAAGGTCAACTCATTCATGAACCTTTATTGC
CAGAAGAATAAGGAGAATAAAAATCTTTACAAATTGCAAAAACTTCACAAACAAATTCTT
TGCATCGCGGATACGTCCTACGAAGTTCCTTACAAATTTGAATCCGATGAGGAAGTGTAT
CAGAGTGTCAATGGATTTTTGGATAATATCTCTTCAAAACATATTGTGGAGAGATTGCGC
AAAATAGGTGATAACTACAATGGCTACAACCTGGACAAGATTTATATTGTTAGCAAGTTC
TATGAAAGTGTCAGTCAAAAGACCTACAGAGATTGGGAGACAATCAACACGGCGCTCGAA
ATACACTACAATAACATCCTCCCCGGCAATGGGAAGAGTAAAGCCGATAAGGTTAAAAAA
GCTGTTAAGAACGACCTCCAGAAATCCATCACGGAAATAAACGAGCTGGTTTCCAACTAT
AAGCTGTGTAGCGATGATAATATTAAGGCTGAGACATATATACATGAGATCAGCCACATT
CTCAACAATTTCGAGGCACAGGAACTCAAATACAATCCCGAGATTCACTTGGTGGAAAGT
GAGTTGAAGGCGTCAGAGCTTAAGAATGTACTTGACGTAATAATGAATGCTTTTCATTGG
TGCTCCGTGTTCATGACTGAGGAACTCGTGGATAAGGATAATAACTTTTATGCGGAGTTG
GAAGAGATATACGATGAAATATACCCGGTTATCTCACTGTATAATCTGGTCAGAAATTAC
GTGACCCAAAAGCCTTATAGTACAAAAAAAATAAAGTTGAACTTCGGTATTCCGACATTG
GCAGATGGTTGGTCCAAAAGCAAAGAATACTCTAATAACGCCATTATATTGATGCGAGAC
AATTTGTATTACCTTGGGATCTTTAACGCGAAAAACAAACCGGATAAGAAGATCATCGAA
GGTAATACATCTGAGAATAAGGGGGATTACAAGAAGATGATTTATAATCTGTTGCCGGGG
CCAAACAAGATGATTCCGAAGGTCTTTCTGTCATCTAAGACAGGAGTAGAGACCTACAAA
CCTTCTGCGTACATTTTGGAAGGCTACAAACAGAACAAGCATATAAAATCTAGCAAGGAC
TTTGATATCACGTTTTGTCATGATCTGATAGATTATTTCAAAAACTGCATCGCTATACAT
CCTGAGTGGAAGAATTTCGGCTTTGACTTTTCTGACACCAGCACATACGAAGACATCTCA
GGTTTCTACCGGGAAGTCGAGCTCCAGGGGTACAAGATTGACTGGACATATATAAGTGAA
AAAGACATCGACCTCCTCCAAGAGAAGGGCCAACTTTACCTGTTCCAGATCTATAACAAA
GACTTTTCTAAAAAGTCCACGGGTAACGACAACTTGCACACTATGTATCTGAAAAACTTG
TTCTCTGAAGAGAACCTCAAGGACATCGTCCTGAAGCTTAACGGGGAGGCGGAGATCTTC
TTTAGAAAGTCCTCTATCAAAAATCCCATTATCCATAAAAAGGGCTCTATACTCGTTAAT
AGGACATATGAAGCGGAGGAAAAAGATCAATTTGGGAACATCCAGATCGTCCGGAAAAAT
ATACCTGAGAATATCTATCAAGAGCTGTACAAGTATTTTAATGATAAGTCAGACAAAGAG
CTCAGTGATGAGGCGGCAAAGCTCAAGAACGTGGTGGGGCATCATGAAGCTGCGACGAAC
ATTGTCAAAGATTATAGATACACTTACGATAAATACTTCCTCCACATGCCGATAACGATT
AACTTCAAAGCCAATAAGACGGGGTTTATAAATGATCGGATCCTTCAGTACATTGCGAAA
GAGAAAGACCTCCATGTGATCGGAATTGACCGAGGAGAAAGGAATCTGATTTACGTGTCC
GTGATTGATACTTGCGGGAATATAGTCGAGCAAAAGAGTTTCAACATAGTCAACGGGTAT
GACTATCAGATAAAGCTCAAACAGCAGGAAGGTGCGAGGCAAATTGCGCGCAAAGAGTGG
AAGGAGATAGGCAAGATTAAAGAAATCAAGGAAGGTTATCTCAGCTTGGTGATCCATGAA
ATATCTAAGATGGTTATAAAGTACAATGCCATAATAGCCATGGAGGATCTTTCCTACGGG
TTTAAGAAGGGCCGATTTAAAGTGGAGCGACAAGTTTACCAGAAGTTCGAAACCATGTTG
ATTAACAAACTTAACTATTTGGTGTTCAAGGATATAAGTATAACCGAAAACGGCGGTTTG
CTTAAGGGTTATCAGCTCACGTATATTCCTGATAAACTTAAAAACGTTGGACACCAGTGT
GGATGTATCTTCTACGTGCCAGCCGCTTACACTAGTAAGATAGATCCTACCACGGGGTTT
GTGAATATTTTTAAGTTTAAAGACTTGACAGTCGACGCCAAAAGGGAATTTATAAAAAAG
TTTGATTCTATCCGCTACGATAGTGAAAAAAATCTCTTTTGCTTTACTTTCGACTATAAC
AACTTCATTACGCAGAACACTGTCATGAGTAAGTCCAGCTGGAGCGTCTACACATATGGC
GTCCGAATTAAACGACGATTTGTAAACGGGCGGTTTTCAAACGAATCTGACACGATAGAC
ATTACCAAGGATATGGAGAAGACACTTGAGATGACCGACATAAACTGGCGGGACGGTCAC
GATCTTCGGCAGGACATAATTGATTACGAAATCGTCCAGCATATATTCGAAATATTTCGA
CTTACAGTGCAAATGCGGAACAGTCTCTCTGAACTGGAAGATCGCGATTATGACCGGTTG
ATTTCTCCGGTCCTCAATGAAAATAACATATTTTATGATAGTGCTAAGGCAGGTGATGCG
TTGCCAAAGGATGCAGACGCTAATGGTGCCTATTGTATCGCGCTCAAGGGATTGTACGAG
ATAAAGCAAATTACGGAGAACTGGAAGGAGGATGGTAAGTTTAGCCGAGACAAGTTGAAG
ATTAGCAATAAAGACTGGTTTGATTTTATCCAAAACAAGAGGTACCTGAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 103
CCAGCGGCTAAAAAAAAGAAACTGGATGGCAGCGTGGATATGAATAACGGAACTAATAAC
TTTCAAAATTTCATAGGTATTTCAAGCTTGCAGAAGACCCTGAGGAATGCCCTGATTCCA
ACCGAGACAACGCAGCAGTTCATAGTCAAAAATGGCATTATTAAGGAAGATGAGCTGCGG
GGGGAAAACCGACAGATACTCAAGGATATTATGGACGACTATTACCGGGGATTTATCTCA
GAAACGCTGAGCAGTATTGATGACATCGATTGGACCAGTCTTTTCGAGAAAATGGAAATT
CAACTTAAGAATGGTGACAATAAAGACACTCTCATAAAGGAGCAAACTGAATACCGAAAA
GCCATACACAAAAAGTTTGCCAACGATGACCGCTTTAAAAACATGTTTTCAGCTAAGCTC
ATTAGCGACATTCTCCCCGAGTTTGTGATTCATAACAATAACTATAGCGCATCCGAGAAG
GAGGAAAAAACCCAAGTTATCAAATTGTTCAGTAGATTCGCTACGAGCTTTAAAGATTAC
TTTAAAAACCGGGCTAACTGCTTCAGTGCAGACGATATCAGCTCCTCATCCTGTCATCGC
ATCGTCAATGATAATGCTGAGATCTTCTTTTCTAATGCACTGGTTTACCGCAGGATAGTT
AAGTCTCTTAGTAACGACGACATCAACAAGATATCAGGAGATATGAAGGATTCCCTTAAA
GAAATGAGTCTCGAGGAGATATATTCTTATGAAAAATACGGCGAATTTATTACCCAAGAG
GGCATTAGTTTCTATAATGACATATGCGGAAAAGTTAATAGTTTTATGAATCTCTATTGT
CAGAAGAATAAGGAGAATAAGAACCTCTACAAATTGCAGAAGTTGCACAAGCAAATTCTG
TGTATCGCGGACACCTCTTACGAGGTCCCATATAAGTTCGAGAGTGATGAAGAAGTATAC
CAGAGCGTTAATGGGTTCCTGGACAACATCTCAAGTAAACACATAGTCGAAAGGCTCCGA
AAGATCGGTGATAACTATAACGGATATAATTTGGATAAAATTTATATAGTTAGCAAATTT
TACGAGAGCGTCAGTCAGAAGACCTACCGGGACTGGGAGACCATAAACACAGCGCTGGAA
ATACATTATAACAACATACTGCCTGGGAACGGTAAGTCAAAGGCAGACAAGGTTAAAAAG
GCTGTGAAGAATGACCTGCAAAAATCAATTACAGAAATAAATGAGTTGGTAAGTAATTAC
AAACTTTGCAGCGATGATAATATAAAGGCAGAGACGTACATACATGAAATATCTCATATC
CTCAACAATTTCGAAGCCCAAGAACTGAAGTACAACCCGGAAATTCATCTTGTAGAGTCT
GAGTTGAAGGCCTCCGAATTGAAAAACGTTCTTGACGTAATTATGAATGCCTTCCACTGG
TGCTCAGTATTCATGACGGAAGAGCTCGTGGATAAAGACAACAATTTTTACGCTGAACTG
GAAGAAATATATGACGAGATTTACCCCGTAATTTCACTCTACAACTTGGTACGAAATTAC
GTTACCCAAAAGCCATACTCAACAAAAAAAATTAAACTGAACTTCGGGATACCCACCCTC
GCAGATGGATGGTCAAAGTCCAAAGAGTACAGTAACAATGCAATTATCCTGATGCGAGAC
AACCTTTATTACCTCGGGATTTTCAACGCTAAAAATAAACCTGATAAAAAAATAATTGAG
GGTAATACCTCTGAAAACAAGGGGGATTATAAAAAGATGATATACAATCTGCTGCCTGGC
CCGAACAAAATGATTCCTAAAGTCTTCTTGTCTTCCAAGACTGGAGTCGAAACCTACAAG
CCAAGTGCTTATATACTCGAAGGGTACAAACAAAATAAGCACATAAAATCCAGCAAGGAT
TTTGATATTACATTCTGCCACGATTTGATTGATTATTTTAAGAACTGTATAGCCATCCAC
CCAGAATGGAAGAATTTTGGTTTTGATTTTAGCGATACCTCAACATATGAGGATATCTCT
GGCTTTTACCGCGAGGTAGAACTGCAAGGTTATAAGATCGATTGGACTTATATTTCTGAA
AAGGACATAGATCTCCTGCAAGAGAAAGGGCAACTTTATTTGTTTCAAATATACAACAAA
GATTTTAGTAAGAAGAGTACTGGCAATGATAACCTTCACACTATGTATCTGAAGAACCTT
TTTTCTGAGGAGAACTTGAAGGACATAGTCCTTAAACTCAATGGGGAAGCTGAAATATTC
TTTCGCAAAAGCTCCATTAAAAACCCGATCATTCATAAAAAGGGTTCCATCTTGGTAAAC
CGCACATACGAGGCGGAAGAAAAAGATCAGTTCGGAAATATCCAGATCGTAAGGAAGAAT
ATCCCCGAAAATATATACCAAGAGCTTTACAAATATTTTAACGATAAGTCAGACAAGGAA
CTGTCAGACGAAGCAGCCAAGTTGAAGAATGTCGTAGGGCACCACGAAGCAGCTACAAAC
ATAGTTAAAGATTATCGGTACACCTACGATAAATATTTCCTGCATATGCCAATAACCATA
AACTTCAAAGCCAACAAAACAGGGTTCATCAATGACCGAATACTTCAGTATATAGCCAAG
GAAAAAGACCTGCATGTTATAGGAATAGATAGAGGTGAGCGCAACTTGATATATGTCAGC
GTGATAGACACCTGCGGAAATATCGTCGAGCAAAAAAGTTTCAACATTGTTAATGGCTAC
GATTACCAAATTAAATTGAAGCAGCAAGAGGGGGCTCGGCAAATCGCGCGAAAGGAATGG
AAAGAAATCGGGAAGATTAAAGAAATTAAAGAGGGCTACCTGTCTCTTGTAATTCACGAA
ATATCTAAGATGGTCATCAAGTATAATGCCATTATTGCGATGGAAGATCTGTCCTACGGA
TTTAAGAAAGGCAGGTTTAAAGTCGAAAGGCAGGTGTACCAGAAATTCGAGACCATGCTG
ATTAATAAGCTCAACTATCTCGTATTTAAGGATATTTCTATAACTGAAAATGGAGGGCTT
CTCAAAGGATATCAACTCACATACATACCTGATAAGCTGAAGAACGTAGGCCACCAGTGT
GGATGCATATTCTATGTACCAGCTGCATACACAAGCAAGATCGATCCAACTACTGGGTTT
GTCAATATCTTCAAATTTAAGGACTTGACGGTCGATGCCAAACGGGAGTTCATCAAAAAG
TTTGATAGTATTCGATATGATAGTGAGAAGAACTTGTTTTGCTTCACATTTGACTACAAC
AATTTCATAACGCAAAATACGGTTATGTCTAAATCCTCATGGAGCGTCTACACTTACGGA
GTGAGGATAAAGCGGCGCTTCGTAAATGGCAGGTTTAGCAATGAATCCGACACGATTGAC
ATAACCAAGGATATGGAGAAAACCCTCGAGATGACCGATATAAATTGGCGGGATGGACAC
GATCTGCGACAAGACATAATCGATTATGAAATCGTGCAGCACATATTTGAGATATTCAGG
CTTACGGTCCAAATGAGAAATTCCCTTTCCGAACTTGAAGACCGCGATTACGACCGACTG
ATAAGCCCCGTTCTGAACGAAAATAACATCTTCTACGACAGCGCTAAAGCGGGAGACGCG
CTGCCGAAAGATGCGGACGCAAATGGAGCCTATTGTATCGCCTTGAAAGGGTTGTACGAG
ATCAAACAGATAACCGAGAATTGGAAGGAGGATGGGAAGTTTAGTCGAGACAAACTTAAA
ATAAGCAACAAGGACTGGTTCGACTTTATTCAAAACAAACGATATCTCAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 104
CCAGCGGCTAAAAAAAAGAAACTGGATGGCAGCGTGGATATGAATAATGGTACTAACAAT
TTTCAAAACTTTATCGGCATCTCTTCACTTCAGAAAACTCTTCGGAACGCCCTTATACCG
ACGGAGACAACGCAGCAGTTTATAGTTAAAAACGGGATCATTAAAGAAGATGAACTCAGA
GGGGAAAACAGGCAAATATTGAAGGACATTATGGACGATTACTACCGGGGGTTTATTTCA
GAGACCCTTTCATCTATTGATGACATAGATTGGACCTCCCTTTTCGAGAAAATGGAGATA
CAATTGAAAAACGGCGACAATAAAGATACACTTATCAAGGAACAAACTGAGTATCGCAAG
GCGATTCACAAGAAGTTTGCGAATGACGATCGCTTTAAGAATATGTTTTCTGCGAAGCTC
ATAAGTGACATTCTGCCTGAATTTGTCATTCATAACAACAATTATTCTGCTAGCGAAAAA
GAGGAAAAAACTCAAGTCATTAAGCTTTTTAGCAGGTTCGCTACTAGTTTTAAAGACTAT
TTTAAGAACCGGGCGAATTGCTTTAGCGCTGACGACATATCATCCTCATCCTGTCATCGC
ATAGTCAATGATAATGCAGAAATATTCTTTTCTAATGCGCTCGTGTATCGGAGAATAGTG
AAAAGCCTCTCTAACGATGACATTAACAAAATAAGCGGCGATATGAAGGATAGTCTGAAG
GAAATGTCCCTCGAAGAAATATACTCATACGAGAAGTACGGAGAATTTATCACCCAGGAA
GGAATTAGTTTTTACAACGACATCTGTGGTAAGGTTAACTCTTTTATGAATCTGTATTGT
CAAAAGAATAAAGAAAATAAAAATCTTTATAAGCTCCAAAAGCTTCACAAACAAATCTTG
TGCATTGCGGATACGTCATACGAAGTACCTTACAAATTTGAAAGCGACGAAGAGGTGTAT
CAGTCAGTGAATGGGTTCCTTGACAATATTTCTAGCAAACATATTGTGGAGCGACTTCGA
AAGATCGGTGATAATTACAATGGCTATAATTTGGATAAAATTTACATAGTTAGTAAGTTT
TATGAATCCGTCTCACAAAAGACGTACCGAGATTGGGAGACCATCAACACTGCTCTGGAG
ATTCATTACAATAATATATTGCCTGGGAATGGGAAGTCAAAGGCCGACAAGGTTAAAAAA
GCCGTAAAAAACGATCTTCAAAAGTCCATTACCGAGATAAATGAACTTGTATCCAACTAT
AAGTTGTGCTCTGACGATAATATTAAAGCAGAAACGTATATCCACGAAATAAGTCACATC
CTGAACAACTTCGAAGCTCAAGAGCTCAAGTATAATCCTGAAATTCATCTCGTCGAAAGC
GAGCTGAAAGCATCCGAGTTGAAGAATGTGCTTGATGTGATCATGAACGCATTCCATTGG
TGCAGTGTGTTCATGACCGAAGAACTTGTAGACAAAGACAACAACTTCTACGCTGAATTG
GAAGAGATTTACGATGAAATTTACCCCGTGATATCCCTCTATAATCTGGTAAGAAATTAC
GTCACGCAAAAACCATACAGTACCAAGAAAATAAAGCTCAACTTTGGTATTCCGACGTTG
GCAGATGGGTGGAGTAAGAGCAAGGAGTATTCTAACAATGCAATCATCCTCATGCGCGAC
AATTTGTATTATCTGGGGATCTTCAACGCGAAAAATAAGCCCGACAAAAAGATAATAGAA
GGCAATACGTCCGAGAACAAAGGGGACTATAAGAAAATGATTTATAACCTTCTTCCAGGA
CCCAACAAGATGATCCCAAAGGTTTTCTTGAGTTCAAAAACCGGCGTAGAAACTTATAAA
CCGTCCGCCTACATTCTGGAAGGGTACAAGCAAAACAAGCACATTAAGTCATCTAAGGAT
TTCGACATTACTTTTTGTCATGATTTGATAGACTACTTCAAAAATTGTATAGCGATACAT
CCGGAATGGAAAAATTTTGGGTTCGATTTTTCCGACACAAGTACTTATGAAGACATCTCA
GGGTTTTATAGGGAAGTTGAACTGCAAGGTTACAAAATAGACTGGACTTATATTAGTGAG
AAGGACATTGATTTGCTCCAGGAAAAGGGTCAATTGTATCTGTTCCAGATATATAACAAG
GATTTCTCTAAAAAATCTACAGGTAACGACAATCTCCACACGATGTACCTCAAGAATCTC
TTCAGCGAAGAGAATTTGAAGGATATCGTACTTAAGCTCAATGGAGAAGCGGAAATATTC
TTCAGAAAGTCCAGCATTAAGAATCCTATAATTCACAAGAAAGGGTCAATTCTCGTAAAC
CGGACTTATGAGGCCGAAGAAAAAGATCAGTTTGGTAACATTCAGATTGTACGGAAAAAC
ATTCCCGAGAACATCTATCAAGAACTGTATAAATACTTTAATGATAAATCCGACAAGGAA
CTTTCTGACGAGGCTGCAAAATTGAAGAACGTAGTGGGACACCATGAGGCCGCAACCAAT
ATAGTAAAGGATTACAGATACACTTATGATAAGTATTTCCTCCATATGCCGATCACGATT
AATTTCAAGGCGAATAAAACCGGCTTCATTAACGATCGCATTTTGCAATATATTGCGAAG
GAAAAGGATTTGCACGTGATAGGTATAGACCGGGGTGAACGAAACTTGATTTACGTCTCT
GTGATCGACACATGCGGAAATATAGTTGAACAGAAGTCCTTTAATATTGTGAATGGTTAC
GACTACCAGATAAAATTGAAGCAACAGGAGGGCGCAAGACAGATAGCTCGCAAAGAGTGG
AAGGAAATCGGCAAGATCAAAGAAATAAAGGAGGGTTATCTTTCCCTGGTAATTCATGAA
ATTAGCAAGATGGTTATTAAGTATAATGCTATAATAGCTATGGAGGACCTTTCCTATGGG
TTCAAGAAAGGTCGCTTCAAAGTGGAGCGACAAGTGTATCAAAAGTTCGAGACTATGTTG
ATAAATAAATTGAATTATTTGGTTTTTAAAGACATTTCAATAACTGAGAACGGGGGTCTC
TTGAAGGGGTACCAATTGACTTATATTCCGGACAAGTTGAAGAATGTCGGACACCAGTGT
GGTTGCATTTTCTACGTGCCTGCCGCTTACACCTCAAAAATCGATCCGACCACTGGTTTT
GTAAATATATTTAAATTCAAAGATCTCACCGTTGATGCCAAACGGGAGTTTATCAAAAAA
TTCGATTCCATTCGCTACGACTCTGAGAAAAACCTTTTTTGTTTCACGTTCGATTATAAC
AACTTTATAACCCAAAATACTGTAATGTCCAAGTCAAGTTGGTCTGTCTATACTTACGGA
GTAAGGATCAAGCGCCGCTTCGTTAATGGGAGATTCTCAAACGAGTCTGATACCATAGAC
ATAACTAAAGACATGGAAAAAACCCTGGAAATGACGGACATCAATTGGCGAGACGGGCAT
GATCTTCGACAGGACATAATAGATTACGAAATTGTTCAACACATTTTCGAGATATTTCGA
CTTACGGTTCAGATGAGGAATTCCCTTTCCGAATTGGAAGACCGGGATTATGATCGACTT
ATATCTCCCGTGCTCAATGAAAACAATATTTTTTATGATTCAGCGAAAGCTGGGGACGCG
CTGCCAAAAGATGCCGATGCCAATGGAGCATACTGTATCGCCCTGAAGGGTTTGTATGAG
ATTAAGCAAATTACTGAAAACTGGAAGGAAGATGGCAAGTTTTCTAGAGATAAGCTTAAG
ATTAGCAATAAGGACTGGTTTGACTTCATTCAAAATAAAAGGTATCTTAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 105
CCAGCGGCTAAAAAAAAGAAACTGGATGGCAGCGTGGATATGAATAATGGAACAAATAAT
TTTCAAAATTTTATTGGTATCAGTTCATTGCAAAAGACTTTGAGAAATGCTTTGATCCCG
ACTGAGACCACACAGCAGTTCATCGTCAAAAATGGCATAATCAAGGAAGACGAACTTAGG
GGTGAGAATAGACAAATATTGAAGGACATCATGGATGACTATTATAGGGGGTTCATTTCC
GAAACGCTCAGTAGTATTGATGACATTGACTGGACTAGTCTTTTCGAGAAAATGGAAATT
CAGCTTAAGAACGGGGACAATAAAGACACGCTGATCAAGGAGCAAACGGAATATAGGAAG
GCGATCCATAAAAAATTCGCGAATGATGATCGGTTTAAAAACATGTTTAGTGCCAAGTTG
ATCAGCGACATACTGCCCGAATTCGTGATCCACAACAATAATTACAGCGCCTCCGAAAAG
GAGGAAAAAACTCAGGTCATTAAATTGTTTAGCCGATTCGCAACGAGTTTCAAAGATTAT
TTTAAGAACCGGGCCAACTGTTTTTCAGCGGATGATATTAGCTCCAGCAGCTGCCATCGC
ATAGTAAATGATAACGCTGAAATCTTTTTTAGCAACGCACTTGTCTACCGGAGGATTGTA
AAATCACTGTCAAATGATGACATTAACAAAATATCTGGAGATATGAAGGACTCACTCAAA
GAAATGAGCCTGGAAGAAATATATTCATACGAAAAATACGGGGAGTTTATTACCCAGGAA
GGTATCAGTTTTTATAATGATATATGTGGAAAAGTTAATTCATTTATGAATCTTTACTGT
CAAAAAAATAAGGAGAACAAGAATTTGTACAAGCTCCAAAAACTTCATAAACAGATTCTG
TGCATCGCAGACACAAGTTATGAGGTACCGTACAAATTTGAGAGCGACGAAGAAGTTTAT
CAGAGTGTGAATGGTTTCCTGGACAATATCTCTTCTAAACACATTGTTGAGAGGCTTAGG
AAGATCGGTGATAATTATAACGGCTATAATCTGGACAAAATTTATATTGTATCAAAGTTT
TATGAATCAGTCTCTCAAAAGACGTATCGGGATTGGGAAACAATTAACACGGCTCTGGAG
ATCCACTACAATAACATTCTGCCCGGCAACGGGAAGAGCAAAGCTGATAAGGTCAAGAAG
GCAGTCAAGAACGACCTTCAGAAGAGCATAACAGAAATTAACGAATTGGTCAGTAACTAC
AAACTGTGTAGTGATGACAACATAAAAGCCGAAACATACATCCATGAAATAAGCCATATC
CTGAATAACTTCGAAGCCCAAGAACTTAAATACAATCCCGAGATTCATCTTGTCGAATCA
GAACTCAAGGCGTCCGAGCTCAAAAATGTCCTTGACGTGATAATGAATGCCTTCCACTGG
TGCAGCGTATTCATGACGGAGGAGTTGGTAGATAAAGACAACAACTTTTATGCCGAATTG
GAAGAGATTTATGATGAGATTTACCCCGTTATTTCTCTGTACAACTTGGTTCGAAACTAC
GTAACACAAAAACCATACTCAACCAAAAAGATCAAACTCAATTTTGGCATACCTACATTG
GCTGATGGTTGGTCCAAGTCAAAGGAATATAGCAATAATGCAATAATTCTCATGCGAGAT
AACTTGTATTATTTGGGGATCTTTAACGCTAAGAACAAACCAGATAAAAAGATAATCGAG
GGGAACACAAGTGAGAACAAGGGTGATTACAAAAAAATGATTTACAATCTGCTTCCTGGG
CCTAACAAAATGATTCCGAAGGTGTTTCTTAGCTCTAAAACTGGAGTGGAGACGTATAAG
CCTTCCGCGTACATTCTCGAAGGCTACAAGCAAAATAAGCATATCAAGTCCAGTAAGGAC
TTCGACATCACTTTTTGCCACGATCTCATCGATTACTTTAAGAACTGTATCGCAATACAC
CCCGAGTGGAAAAACTTTGGTTTTGATTTTTCAGACACTAGTACCTACGAGGACATTTCC
GGCTTCTATCGAGAAGTCGAACTCCAGGGCTACAAAATCGATTGGACGTACATTTCTGAG
AAGGACATCGACTTGCTCCAAGAGAAAGGTCAACTTTACCTCTTCCAAATTTACAATAAA
GACTTTTCAAAGAAGAGCACCGGTAATGACAACTTGCATACCATGTATCTGAAGAACCTG
TTTTCTGAGGAGAACCTCAAGGATATTGTATTGAAGTTGAATGGCGAAGCAGAAATATTT
TTCCGAAAGTCATCTATCAAGAACCCCATTATACACAAAAAAGGCTCTATCCTGGTGAAC
CGGACTTACGAGGCAGAGGAGAAGGATCAATTCGGAAACATACAGATAGTCCGCAAAAAC
ATCCCTGAGAATATCTATCAGGAACTCTATAAGTACTTCAATGATAAATCAGACAAGGAG
CTTAGCGACGAAGCAGCTAAACTTAAAAACGTGGTTGGCCATCACGAGGCCGCTACCAAC
ATAGTCAAAGACTACCGCTATACTTATGACAAGTACTTTTTGCACATGCCCATAACAATT
AATTTCAAAGCTAACAAAACAGGGTTTATAAATGACAGAATCCTCCAATACATCGCCAAA
GAGAAGGACCTCCATGTAATCGGGATTGATAGAGGCGAACGGAACTTGATTTACGTTAGT
GTCATTGATACCTGTGGTAACATTGTCGAACAAAAGTCATTCAACATAGTCAATGGATAT
GATTATCAGATAAAACTCAAGCAACAAGAAGGCGCGAGGCAGATTGCCAGGAAGGAATGG
AAAGAAATCGGGAAGATCAAGGAGATCAAGGAGGGTTACCTGTCCTTGGTGATACACGAG
ATTTCAAAAATGGTTATAAAATACAATGCCATTATCGCGATGGAGGATTTGTCTTATGGA
TTTAAGAAGGGGAGGTTCAAAGTCGAACGACAAGTCTATCAGAAGTTTGAAACAATGCTC
ATTAACAAGCTCAATTACCTTGTTTTCAAGGATATAAGCATCACTGAAAACGGCGGACTC
CTTAAGGGATATCAGCTGACTTATATCCCCGACAAGCTCAAGAACGTAGGGCACCAATGC
GGATGCATCTTTTACGTGCCTGCAGCATATACTTCAAAAATTGATCCGACTACTGGCTTT
GTTAACATTTTCAAGTTCAAGGATCTGACGGTAGACGCTAAGAGAGAATTCATAAAAAAG
TTTGACAGCATCAGGTACGATAGTGAAAAGAACCTTTTTTGTTTTACCTTTGACTACAAT
AATTTTATTACGCAAAATACAGTTATGAGCAAATCAAGTTGGAGCGTTTACACATATGGC
GTTCGGATCAAGCGCAGATTCGTCAATGGTCGCTTCTCAAATGAGAGCGATACAATCGAT
ATAACGAAGGATATGGAGAAGACGCTTGAGATGACAGATATCAACTGGCGGGACGGACAT
GACCTTAGACAAGACATAATCGATTACGAAATAGTACAGCATATCTTTGAGATTTTTAGG
CTTACAGTTCAGATGCGGAACTCTCTTTCCGAACTGGAGGACCGGGATTATGATCGGTTG
ATCTCCCCAGTACTGAACGAAAATAATATCTTTTACGATAGCGCGAAGGCTGGTGATGCA
CTCCCAAAAGACGCTGATGCGAACGGAGCTTATTGCATAGCCCTTAAAGGGCTTTACGAG
ATTAAACAAATAACAGAAAATTGGAAGGAAGATGGCAAATTTTCCCGCGACAAGTTGAAG
ATTAGTAACAAAGACTGGTTCGACTTCATTCAGAATAAACGCTACCTCAAACGTCCGGCA
GCGACCAAAAAAGCCGGCCAGGCGAAGAAAAAAAAAGCGTCAGGTAGCGGCGCAGGCAGC
CCGAAAAAGAAACGTAAAGTCGAGGATCCGAAAAAGAAACGTAAGGTTATTCCGGGCTAA
SEQ ID NO: 113
ATGGGCCATCATCATCATCATCATAGCAGCGGCGTGGATCTGGGCACCGAAAACCTGTAT
TTTCAGTCCATGAGCCGCCGCCGCAAAGCGAACCCGACCAAACTGAGCGAAAACGCGAAA
AAACTGGCGAAAGAAGTGGAAAACGCAAGCGGCAGCGGCGCGGGCAGCAAACGACCGGCG
GCGACCAAAAAAGCGGGCCAAGCGAAGAAAAAGAAAGCAAGCGGCAGCGGCGCGGGCAGC
CCGGCGGCAAAAAAAAAAAAACTGGACGGCAGCGTGGATGCAAGCGGCAGCGGCGCGGGC
AGCCCCAAAAAAAAACGCAAAGTTGAAGATGCAAGCGGCAGCGGCGCGGGCAGCCCGAAA
AAAAAACGTAAAGTGGCAAGCGGCAGCGGCGCGGGCAGCATGAACAACGGCACCAACAAC
TTTCAGAACTTTATTGGCATTAGCAGCCTGCAGAAAACCCTGCGCAACGCGCTGATTCCG
ACCGAAACCACGCAGCAGTTTATTGTGAAAAACGGCATTATTAAAGAAGATGAACTGCGC
GGCGAAAACCGTCAGATTCTGAAGGACATTATGGATGATTATTATCGCGGCTTTATTAGC
GAAACCCTGAGCAGCATTGATGATATAGACTGGACGAGCCTGTTTGAAAAAATGGAAATT
CAGCTGAAAAACGGCGATAACAAAGATACCCTGATTAAAGAACAGACCGAATATCGCAAA
GCGATTCATAAGAAGTTTGCGAACGATGATCGCTTTAAAAACATGTTTAGCGCGAAACTG
ATTAGCGATATTCTGCCGGAATTTGTGATTCATAACAACAACTATAGCGCGAGCGAAAAG
GAAGAAAAAACCCAAGTGATTAAACTGTTTAGCCGCTTTGCGACGAGCTTTAAAGATTAT
TTTAAAAATCGCGCGAACTGCTTTAGCGCGGATGATATTAGCAGCAGCAGCTGCCATCGC
ATTGTGAACGATAACGCGGAGATCTTTTTTAGCAATGCGCTGGTGTATCGCCGCATTGTG
AAAAGCCTGAGCAACGATGATATTAACAAAATTAGCGGCGATATGAAAGATAGCCTGAAA
GAAATGAGCCTGGAAGAAATATATAGCTATGAAAAATATGGGGAATTTATTACACAAGAG
GGCATTAGCTTTTATAACGATATTTGCGGCAAAGTGAACAGCTTTATGAACCTGTATTGT
CAGAAAAACAAAGAAAACAAAAACCTGTATAAACTGCAGAAACTGCATAAACAGATTCTG
TGCATTGCGGATACGAGCTATGAAGTGCCGTATAAATTTGAAAGCGATGAAGAAGTGTAT
CAGAGCGTGAACGGCTTTCTGGATAACATTAGCAGCAAACATATTGTGGAACGCCTGCGC
AAAATTGGCGATAACTATAACGGCTATAACCTGGATAAAATTTATATTGTGAGCAAATTT
TATGAAAGCGTGAGTCAGAAAACCTATCGCGATTGGGAAACCATTAACACCGCGCTGGAA
ATTCATTATAACAACATTCTGCCGGGCAACGGCAAAAGTAAAGCGGATAAAGTGAAAAAA
GCGGTGAAAAACGATCTGCAGAAAAGCATTACGGAAATTAACGAACTGGTGAGCAACTAT
AAACTGTGCAGCGATGATAACATTAAAGCGGAAACCTATATTCACGAGATCAGTCATATT
CTGAACAACTTTGAAGCGCAAGAACTGAAATATAACCCGGAAATTCATCTGGTGGAATCA
GAACTGAAGGCGAGCGAACTTAAGAATGTGCTAGATGTGATTATGAACGCGTTTCATTGG
TGCAGCGTGTTTATGACCGAAGAACTGGTGGATAAAGATAACAACTTTTATGCGGAACTG
GAAGAAATCTACGACGAAATTTATCCGGTGATTAGCCTGTATAACCTGGTGCGCAACTAT
GTGACGCAGAAACCGTATAGCACCAAAAAAATTAAACTGAACTTTGGCATTCCGACCCTG
GCGGATGGCTGGAGCAAGAGCAAAGAGTATAGCAACAACGCTATTATCCTAATGCGCGAT
AACCTGTATTATCTGGGCATTTTTAACGCGAAAAACAAACCGGATAAAAAAATTATTGAA
GGCAACACGAGCGAAAACAAAGGCGATTATAAAAAAATGATTTATAACCTGCTGCCGGGC
CCGAACAAAATGATTCCGAAAGTGTTTCTGAGCAGCAAAACCGGCGTGGAAACCTATAAA
CCGAGCGCGTATATTCTGGAAGGCTATAAACAGAACAAACATATTAAAAGCAGCAAAGAT
TTTGATATTACCTTTTGCCATGATCTGATTGACTACTTTAAGAACTGTATAGCGATTCAT
CCGGAATGGAAAAACTTTGGCTTTGATTTTAGCGATACGAGCACCTATGAAGACATTAGC
GGCTTTTATCGCGAAGTGGAACTGCAAGGCTATAAAATTGATTGGACCTATATTAGCGAA
AAAGATATTGATCTGCTGCAAGAAAAAGGTCAGCTGTATCTGTTTCAGATTTATAACAAA
GATTTTAGCAAAAAAAGCACCGGCAACGATAACCTGCATACCATGTATCTGAAAAATCTG
TTTTCTGAAGAAAACCTAAAAGATATTGTCCTGAAACTGAACGGCGAAGCCGAAATTTTT
TTTCGCAAGAGCAGCATTAAAAACCCGATTATTCACAAAAAAGGTAGCATTCTGGTGAAC
CGCACATACGAAGCTGAGGAAAAGGATCAGTTTGGCAACATTCAGATTGTGCGCAAAAAC
ATTCCGGAAAACATCTACCAAGAACTGTACAAATATTTTAACGATAAAAGCGATAAAGAA
CTGAGCGACGAGGCTGCGAAGCTGAAGAATGTCGTGGGCCATCATGAAGCGGCGACTAAC
ATTGTCAAAGATTATCGCTATACCTATGATAAATATTTTCTGCATATGCCGATTACCATT
AACTTTAAAGCGAACAAAACCGGCTTTATTAACGATCGCATTCTGCAGTATATTGCGAAG
GAAAAGGATCTGCACGTGATTGGCATTGATCGCGGCGAACGCAACCTGATTTATGTGAGC
GTGATTGATACCTGCGGCAACATTGTGGAACAGAAAAGCTTTAACATCGTGAACGGCTAT
GATTATCAGATTAAACTGAAACAGCAAGAAGGCGCGCGTCAGATTGCGCGCAAAGAATGG
AAAGAAATTGGCAAAATTAAAGAAATTAAAGAAGGCTATCTGAGCCTGGTGATTCATGAA
ATCAGCAAGATGGTGATTAAATATAATGCCATTATTGCGATGGAAGATCTGAGCTATGGC
TTTAAAAAAGGCCGCTTTAAAGTGGAACGCCAAGTGTATCAGAAATTTGAAACCATGCTG
ATTAACAAACTGAACTATCTGGTGTTTAAAGATATTAGTATTACTGAAAATGGCGGCCTG
CTGAAAGGCTATCAGCTGACCTATATTCCGGACAAGCTGAAGAATGTGGGCCATCAGTGC
GGCTGCATTTTTTATGTGCCGGCGGCGTATACGAGCAAAATTGATCCGACCACCGGCTTT
GTGAACATTTTTAAATTTAAAGATCTGACCGTGGATGCGAAACGGGAATTCATAAAAAAA
TTTGATAGCATTCGCTATGATAGCGAAAAGAATCTGTTTTGCTTCACCTTTGATTATAAC
AACTTTATAACGCAGAACACCGTGATGAGCAAAAGCAGCTGGAGCGTGTATACCTATGGC
GTGCGCATTAAACGCCGCTTTGTGAACGGCCGCTTTAGCAACGAAAGCGATACCATTGAT
ATTACCAAAGATATGGAAAAAACCCTGGAAATGACCGATATTAACTGGCGCGATGGCCAT
GATCTGCGCCAAGATATTATTGATTATGAAATTGTGCAGCATATTTTTGAAATTTTTCGC
CTGACCGTGCAGATGCGCAACAGCCTGAGCGAACTGGAAGATCGCGATTATGATCGCCTG
ATTAGCCCGGTGCTGAACGAAAACAACATTTTTTATGATAGCGCGAAAGCGGGCGATGCG
CTGCCGAAAGATGCGGATGCGAACGGCGCGTATTGCATTGCGCTGAAAGGCCTGTATGAA
ATTAAACAGATTACGGAAAACTGGAAAGAAGATGGCAAATTTAGCCGCGACAAGCTGAAA
ATTAGCAACAAAGATTGGTTTGATTTTATTCAGAACAAACGCTATCTGTA

In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO:2. In certain embodiments, a nucleic acid-guided nuclease, e.g., Type V, preferably Type VA CRISPR nuclease polypeptide disclosed herein includes a polypeptide having an amino acid sequence of at least about 60%, 65%, 75%, 85%, 95%, 99% or about 100% identity, preferably at least 80%, more preferably at least 90%, even more preferably at least 95%, still more preferably at least 98% identical to amino acid sequence of SEQ ID NO:2. In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO: 3. In certain embodiments, a nucleic acid-guided nuclease, e.g., Type V, preferably Type VA CRISPR nuclease polypeptide disclosed herein includes a polypeptide having an amino acid sequence of at least about 60%, 65%, 75%, 85%, 95%, 99% or about 100% identity, preferably at least 80%, more preferably at least 90%, even more preferably at least 95%, still more preferably at least 98% identical, to amino acid sequence of SEQ ID NO: 3. In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO: 4. In certain embodiments, a nucleic acid-guided nuclease disclosed herein includes a polypeptide having an amino acid sequence of at least about 60%, 65%, 75%, 85%, 95%, 99% or about 100% identity, preferably at least 80%, more preferably at least 90%, even more preferably at least 95%, still more preferably at least 98% identical to amino acid sequences of SEQ ID NO: 4. In certain embodiments, a nucleic acid-guided nuclease, e.g., Type V, preferably Type VA CRISPR nuclease polypeptide disclosed herein includes a polypeptide having an amino acid sequence of at least 50% identity to any one of SEQ ID NOs: 109-112. In certain embodiments, a nucleic acid-guided nuclease disclosed herein includes a polypeptide having an amino acid sequence at least 60, 70, 80, 85, 90, 95, 98, 99%, or 100%, identical, preferably at least 80%, more preferably at least 90%, even more preferably at least 95%, still more preferably at least 98% identical to amino acid sequence of any one of SEQ ID NOs: 109-112. In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO: 109. In certain embodiments, a nucleic acid-guided nuclease disclosed herein includes a polypeptide having an amino acid sequence at least 60, 70, 80, 85, 90, 95, 98, 99%, or 100%, identical, preferably at least 80%, more preferably at least 90%, even more preferably at least 95%, still more preferably at least 98% identical to SEQ ID NO: 109. In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO: 110. In certain embodiments, a nucleic acid-guided nuclease disclosed herein includes a polypeptide having an amino acid sequence at least 60, 70, 80, 85, 90, 95, 98, 99%, or 100%, identical, preferably at least 80%, more preferably at least 90%, even more preferably at least 95%, still more preferably at least 98% identical to SEQ ID NO: 110. In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO: 111. In certain embodiments, a nucleic acid-guided nuclease disclosed herein includes a polypeptide having an amino acid sequence at least 60, 70, 80, 85, 90, 95, 98, 99%, or 100%, identical, preferably at least 80%, more preferably at least 90%, even more preferably at least 95%, still more preferably at least 98% identical to SEQ ID NO: 111. In certain embodiments, a nucleic acid-guided nuclease, e.g., Type V, preferably Type VA CRISPR nuclease polypeptide disclosed herein includes a polypeptide having an amino acid sequence of at least 50% identity to SEQ ID NO: 112. In certain embodiments, a nucleic acid-guided nuclease disclosed herein includes a polypeptide having an amino acid sequence at least 60, 70, 80, 85, 90, 95, 98, 99%, or 100%, identical, preferably at least 80%, more preferably at least 90%, even more preferably at least 95%, still more preferably at least 98% identical to SEQ ID NO: 112.

### Nuclear Localization Signals (NLSs)

In certain embodiments, a composition, e.g., nuclease, disclosed herein includes one or more nuclear localization sequences (NLSs), such as 1, 2, 3, 4, 5, 6, 7, 8, 9, 10, or more NLSs. In some embodiments, a composition, e.g., engineered nuclease comprises 1, 2, 3, 4, 5, 6, 7, 8, 9, 10, or more NLSs at or near the amino-terminus, 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In certain embodiments the engineered nuclease comprises 4 NLSs.

Non-limiting examples of NLSs include an NLS sequence derived from: the NLS of the SV40 virus large T-antigen, having the amino acid sequence PKKKRKV (SEQ ID NO:5); the NLS from nucleoplasmin (e.g. the nucleoplasmin bipartite NLS with the sequence KRPAATKKAGQAKKKK (SEQ ID NO:6); the c-myc NLS having the amino acid sequence PAAKRVKLD SEQ ID NO:7) or RQRRNELKRSP (SEQ ID NO:8); the hRNPA1 M9 NLS having the sequence NQSSNFGPMKGGNFGGRSSGPYGGGGQYFAKPRNQGGY (SEQ ID NO: 9); the sequence RMRIZFKNKGKDTAELRRRRVEVSVELRKAKKDEQILKRRNV (SEQ ID NO:10) of the IBB domain from importin-alpha; the sequences VSRKRPRP (SEQ ID NO: 11) and PPKKARED (SEQ ID NO:12) of the myoma T protein; the sequence PQPKKKPL (SEQ ID NO:13) of human p53; the sequence SALIKKKKKMAP (SEQ ID NO:14) of mouse c-abl IV; the sequences DRLRR (SEQ ID NO:15) and PKQKKRK (SEQ ID NO:16) of the influenza virus NS1; the sequence RKLKKKIKKL (SEQ ID NO:17) of the Hepatitis virus delta antigen; the sequence REKKKFLKRR (SEQ ID NO:18) of the mouse Mx1 protein; the sequence KRKGDEVDGVDEVAKKKSKK (SEQ ID NO:19) of the human poly(ADP-ribose) polymerase; the sequence RKCLQAGMNLEARKTKK (SEQ ID NO:20) of the steroid hormone receptors (human) glucocorticoid; and EGL-13, MSRRRKANPTKLSENAKKLAKEVEN, SEQ ID NO: 107.

In certain embodiments, a nuclease provided herein comprises at least one myc-related NLS comprising the sequence PAAKKKKLD (SEQ ID NO:21); in certain embodiments the myc-related NLS is at the N-terminus of the nuclease. In certain embodiments, a nuclease provided herein comprises at least one nucleoplasmin NLS comprising the sequence KRPAATKKAGQAKKKK (SEQ ID NO:6); in certain embodiments the nucleoplasmin NLS is at the C-terminus of the nuclease. In certain embodiments a nuclease provided herein comprises at least one, or at least two, SV40 NLS sequences comprising the sequence PKKKRKV (SEQ ID NO: 5); in certain embodiments the SV40 NLSs are at the C-terminus of the nuclease. In certain embodiments, a nuclease provided herein comprises 1 NLS at the N-terminus and 3 NLSs at the C-terminus, for example 1 myc-related NLS at the N-terminus and one nucleoplasmin NLS and two SV40 NLSs at the C-terminus. In certain embodiments, a nuclease provided herein comprises 1 myc-related NLS at the N-terminus with the sequence PAAKKKKLD (SEQ ID NO: 21 and one nucleoplasmin NLS comprising the sequence KRPAATKKAGQAKKKK (SEQ ID NO: 6) and two SV40 NLSs comprising the sequence PKKKRKV (SEQ ID NO:5) at the C-terminus.

In general, the one or more NLSs are of sufficient strength to drive accumulation of the nucleic acid-guided nuclease in a detectable amount in the nucleus of a eukaryotic cell. In general, strength of nuclear localization activity may derive from the number of NLSs, the particular NLS(s) used, or a combination of these factors. Detection of accumulation in the nucleus may be performed by any suitable technique. For example, a detectable marker may be fused to the nucleic acid-guided nuclease, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the nucleic acid-guided nuclease complex formation (e.g. assay for DNA cleavage or mutation at the target sequence, or assay for altered gene expression activity affected by targetable nuclease complex formation and/or nucleic acid-guided nuclease activity), as compared to a control not exposed to the nucleic acid-guided nuclease or targetable nuclease complex, or exposed to a nucleic acid-guided nuclease lacking the one or more NLSs.

In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4 and at least one myc-related NLS comprising the sequence PAAKKKKLD (SEQ ID NO:21); in certain embodiments the myc-related NLS is at the N-terminus of the nuclease. In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4 and at least one nucleoplasmin NLS comprising the sequence KRPAATKKAGQAKKKK (SEQ ID NO:6); in certain embodiments the nucleoplasmin NLS is at the C-terminus of the nuclease. In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4 and at least one, or at least two, SV40 NLS sequences comprising the sequence PKKKRKV (SEQ ID NO: 5); in certain embodiments the SV40 NLSs are at the C-terminus of the nuclease. In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4 and one NLS at the N-terminus and three NLSs at the C-terminus, for example 1 myc-related NLS at the N-terminus and one nucleoplasmin NLS and two SV40 NLSs at the C-terminus. In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4, and one myc-related NLS at the N-terminus with the sequence PAAKKKKLD (SEQ ID NO:21) and one nucleoplasmin NLS comprising the sequence KRPAATKKAGQAKKKK (SEQ ID NO:6) and two SV40 NLSs comprising the sequence PKKKRKV (SEQ ID NO:5) at the C-terminus. In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 1, and one, two, or three NLS at the N-terminus and one, two, or three NLS at the C-terminus. In certain embodiments, a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 1, and one myc-related NLS at the N-terminus with the sequence PAAKKKKLD (SEQ ID NO:21) and one nucleoplasmin NLS comprising the sequence KRPAATKKAGQAKKKK (SEQ ID NO:6) and two SV40 NLSs comprising the sequence PKKKRKV (SEQ ID NO:5) at the C-terminus.

### Purification Tags

In certain embodiments, a nucleic acid-guided nuclease provided herein can comprise a tag, e.g., a purification tag, e.g. at the N-terminus. Exemplary tags include a poly-his tag, such as a Gly-6×His tag (SEQ ID NO: 421) or Gly-8×His tag (SEQ ID NO: 422), short epitope tags such as FLAG, hemagglutinin (HA), c-myc, T7, and Glu-Glu; maltose binding protein (mbp); N-terminal glutathione S-transferase (GST); calmodulin binding peptide (CBP). In certain embodiments, a nucleic acid-guided nuclease provided herein can comprise a poly-his tag, such as a Gly-6×His tag (SEQ ID NO: 421), e.g., at the N-terminus. These Gly-6×His tags (SEQ ID NO: 421) are applied for several reasons including: 1) a 6×His tag (SEQ ID NO: 423) can be used in protein purification to allow binding to the chromatographic columns for purification, and 2) the N-terminal glycine allows further, site-specific, chemical modifications that permit advanced protein engineering. Further, the Gly-6×His (SEQ ID NO: 421) is designed for easy removal, if desired, by digestion with Tobacco Etch Virus (TEV) protease. For these constructs, the Gly-6×His tag (SEQ ID NO: 421) was positioned on the N-terminus. Gly-6×His tags (SEQ ID NO: 421) are further described in Martos-Maldonado et al., Nat Commun. (2018) 17;9 (1): 3307, the disclosure of which is incorporated herein. Thus, in certain embodiments provided herein is a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4 and a poly-His tag at the N-terminus, such as a Gly-6×His tag (SEQ ID NO: 421). In certain embodiments provided herein is a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 4, a poly-His tag at the N-terminus, such as a Gly-6×His tag (SEQ ID NO: 421), and/or a TEV cleavage site at the N-terminus. In certain embodiments provided herein is a nucleic acid-guided nuclease having a poly-His tag at the N-terminus, such as a Gly-6×His tag (SEQ ID NO: 421) and a TEV cleavage site at the N-terminus, such as a polypeptide having at least about 60%, 65%, 75%, 85%, 95%, 99% or about 100% identity to amino acid sequences of SEQ ID NO: 2. In certain embodiments provided herein is a nucleic acid-guided nuclease disclosed herein includes a polypeptide having an amino acid sequence of at least about 60%, 65%, 75%, 85%, 95%, 99% or about 100% identity to amino acid sequences of SEQ ID NO: 1, a poly-His tag at the N-terminus, such as a Gly-6×His tag (SEQ ID NO: 421), and/or a TEV cleavage site at the N-terminus. Additionally or alternatively, the nuclease may comprise one or more NLS as described herein.

### Cleavage Sites

In addition to, or alternatively to, including one or more NLSs, purification tags, and/or other additional amino acid sequences described herein, an engineered nuclease polypeptide disclosed herein can include one or more cleavage sites, which can be at or near the N-terminus or the C-terminus. Any suitable cleavage site can be used; if a plurality of cleavage sits is used, they may be the same or different. In certain embodiments a cleavage site comprises a Tobacco Etch Virus protease cleavage sequence, herein referred to as a “TEV sequence” (SEQ ID NO: 108). The TEV sequence can be at or near the amino terminus. Generally, the cleavage sequence, e.g., TEV sequence, is located so that cleavage at the cleavage sequence leaves other additional amino acid sequences, in particular any NLS added to the original nuclease polypeptide, intact. A TEV clevage site can have the amino acid sequence ENLYFQS (SEQ ID. NO: 108.

In certain embodiments, provided herein is a nucleic acid sequence encoding a polypeptide having at least 50% nucleic acid identity to a polypeptide represented by SEQ ID NO: 2. In certain embodiments, provided herein is a nucleic acid sequence encoding a polypeptide having at least about 60%, 65%, 70%, 75%, 80%, 85%, 90%, 95%, greater than 95%, or 100% to a polypeptide represented by SEQ ID NO: 2. In certain embodiments, provided herein is a nucleic acid sequence encoding a polypeptide having at least at least 50% nucleic acid identity to a polypeptide represented by SEQ ID NO: 3. In certain embodiments, provided herein is a nucleic acid sequence encoding a polypeptide having at least about 60%, 65%, 70%, 75%, 80%, 85%, 90%, 95%, greater than 95%, or 100% to a polypeptide represented by SEQ ID NO: 3. In certain embodiments, provided herein is a nucleic acid sequence encoding a polypeptide having at least at least 50% nucleic acid identity to a polypeptide represented by SEQ ID NO: 4. In certain embodiments, provided herein is a nucleic acid sequence encoding a polypeptide having at least about 60%, 65%, 70%, 75%, 80%, 85%, 90%, 95%, greater than 95%, or 100% to a polypeptide represented by SEQ ID NO: 4. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 23-105. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 23-42 In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 43-65. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 43-53. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 54-58. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 59-63. In certain embodiments, provided herein is a nucleic acid of at least about 50%, 60%, 65%, 70%, 75%, 80%, 85%, 90%, 95%, 99%, or 100% polynucleotide identity to any one of SEQ ID NO: 43. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 64-84. In certain embodiments, provided herein is a nucleic acid of at least about 50%, 60%, 65%, 70%, 75%, 80%, 85%, 90%, 95%, 99%, or 100% polynucleotide identity to any one of SEQ ID NO: 64. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 64-74. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 75-79. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 80-84. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 85-105. In certain embodiments, provided herein is a nucleic acid of at least about 50%, 60%, 65%, 70%, 75%, 80%, 85%, 90%, 95%, 99%, or 100% polynucleotide identity to any one of SEQ ID NO: 85. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 85-95. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 96-100. In certain embodiments, provided herein is a nucleic acid of at least about 50%, 60%, 65%, 70%, 75%, 80%, 85%, 90%, 95%, 99%, or 100% polynucleotide identity to any one of SEQ ID NOS: 101-105.

A nucleic acid sequence encoding a nucleic acid-guided nuclease can be operably linked to a promoter. Such nucleic acid sequences can be linear or circular. The nucleic acid sequences can be encompassed on a larger linear or circular nucleic acid sequences that comprises additional elements such as an origin of replication, selectable or screenable marker, terminator, other components of a targetable nuclease system, such as a guide nucleic acid, and/or an editing or recorder cassette as disclosed herein. In some aspects, nucleic acid sequences can include sequences that code for at least one glycine, at least one poly-histidine tag, such as a 6×histidine tag (SEQ ID NO: 423), and/or at least one, two, three, four, or five nuclear localization signal tags, some or all of which can be on the amino side of the polypeptide, the carboxy side of the polypeptide, or a combination thereof. Larger nucleic acid sequences can be recombinant expression vectors, as are described in more detail later.

### Guide Nucleic Acids

In certain embodiments, compositions and methods disclosed herein include a guide nucleic acid (gNA), e.g., a gRNA.

In general, a guide polynucleotide, also referred to as a guide nucleic acid (gNA) can complex with a compatible nucleic acid-guided nuclease, such as those disclosed herein, and can hybridize with a target nucleic acid sequence, thereby directing the nuclease to the target nucleic acid sequence. A subject nucleic acid-guided nuclease capable of complexing with a guide polynucleotide can be referred to as a nucleic acid-guided nuclease that is compatible with the guide polynucleotide. In addition, a guide polynucleotide capable of complexing with a nucleic acid-guided nuclease can be referred to as a guide polynucleotide or a guide nucleic acid that is compatible with the nucleic acid-guided nuclease. In some embodiments, a polynucleotide (gRNA) disclosed herein can be split into fragments, e.g., two separate polynucleotides, in some cases encompassing a synthetic tracrRNA and crRNA. Such gNAs, e.g., gRNAs, can be referred to as dual or split gNA, e.g., gRNA.

A guide polynucleotide can be DNA. A guide polynucleotide can be RNA. A guide polynucleotide can include both DNA and RNA. A guide polynucleotide can include modified or non-naturally occurring nucleotides. In cases where the guide polynucleotide comprises RNA, the RNA guide polynucleotide can be encoded by a DNA sequence on a polynucleotide molecule such as a plasmid, linear construct, or editing cassette as disclosed herein.

A guide polynucleotide can comprise a guide sequence, also referred to herein as a spacer sequence. A guide (spacer) sequence is a polynucleotide sequence having sufficient complementarity with a target polynucleotide sequence, also referred to herein as a target nucleic acid sequence, to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In other embodiments, a guide sequence can be less than about 75, 50, 45, 40, 35, 30, 25, 20 nucleotides in length. Preferably the guide sequence is 10-30 nucleotides long. The guide sequence can be 15-20 nucleotides in length. The guide sequence can be 15 nucleotides in length. The guide sequence can be 16 nucleotides in length. The guide sequence can be 17 nucleotides in length. The guide sequence can be 18 nucleotides in length. The guide sequence can be 19 nucleotides in length. The guide sequence can be 20 nucleotides in length.

A guide polynucleotide can include a scaffold sequence. In general, a “scaffold sequence” can include any sequence that has sufficient sequence to promote formation of a targetable nuclease complex, wherein the targetable nuclease complex includes, but is not limited to, a nucleic acid-guided nuclease and a guide polynucleotide that can include a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are included or encoded on the same polynucleotide. In some cases, the one or two sequence regions are included or encoded on separate polynucleotides. Optimal alignment may be determined by any suitable alignment algorithm, and may further account for secondary structures, such as self-complementarity within either the one or two sequence regions. In some embodiments, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions can be about or more than about 5, 6, 7, 8, 9, 10, 11, 12, 13, 14, 15, 16, 17, 18, 19, 20, 25, 30, 40, 50, or more nucleotides in length.

A scaffold sequence of a subject guide polynucleotide can comprise a secondary structure. A secondary structure can comprise a pseudoknot region. In some cases, binding kinetics of a guide polynucleotide to a nucleic acid-guided nuclease is determined in part by secondary structures within the scaffold sequence. In some cases, binding kinetics of a guide polynucleotide to a nucleic acid-guided nuclease is determined in part by nucleic acid sequence with the scaffold sequence. In some aspects, the invention provides a nuclease that binds to a guide polynucleotide can include a conserved scaffold sequence. For example, the nucleic acid-guided nucleases for use in the present disclosure can bind to a conserved pseudoknot region.

In certain embodiments, the engineered polynucleotide (gRNA) can be split into fragments encompassing a synthetic tracrRNA and crRNA.

As used herein, “guide nucleic acid” or “guide polynucleotide” can refer to one or more polynucleotides and can include 1) a guide (spacer) sequence capable of hybridizing to a target sequence and 2) a scaffold sequence capable of interacting with or complexing with a nucleic acid-guided nuclease as described herein. A guide nucleic acid can be provided as one or more nucleic acids. In specific embodiments, the guide sequence and the scaffold sequence are provided as a single polynucleotide. In other aspects, guide nucleic acid may include at least one amplicon targeting fragments.

A guide nucleic acid can be compatible with a nucleic acid-guided nuclease when the two elements can form a functional targetable nuclease complex capable of cleaving a target sequence. In certain methods, a compatible scaffold sequence for a compatible guide nucleic acid can be found by scanning sequences adjacent to a native nucleic acid-guided nuclease loci. For example, native nucleic acid-guided nucleases can be encoded on a genome within proximity to a corresponding compatible guide nucleic acid or scaffold sequence.

Nucleic acid-guided nucleases can be compatible with guide nucleic acids that are not found within the nucleases endogenous host. Such orthogonal guide nucleic acids can be determined by empirical testing. Orthogonal guide nucleic acids can come from different bacterial species or be synthetic or otherwise engineered to be non-naturally occurring.

Orthogonal guide nucleic acids that are compatible with a common nucleic acid-guided nuclease can comprise one or more common features. Common features can include sequence outside a pseudoknot region. Common features can include a pseudoknot region. Common features can include a primary sequence or secondary structure.

A guide nucleic acid can be engineered to target a desired target sequence by altering the guide (spacer) sequence such that the guide sequence is complementary to the target sequence, thereby allowing hybridization between the guide sequence and the target sequence. A guide nucleic acid with an engineered guide sequence can be referred to as an engineered guide nucleic acid. Engineered guide nucleic acids are often non-naturally occurring and are not found in nature.

Engineered guide nucleic acids can be formed using a Synthetic Tracr RNA (STAR) system. STAR, when combined with a Cas12a protein, can form at least one ribonucleoprotein (RNP) complex that targets a specific genomic locus. STAR takes advantage of the natural properties of the CRISPR (Clustered Regularly Interspaced Short Palindromic Repeats) where the CRISPR system functions much like an immune system against invading viruses and plasmid DNA. Short DNA sequences (spacers) from invading viruses are incorporated at CRISPR loci within the bacterial genome and serve as “memory” of previous infections. Reinfection triggers complementary mature CRISPR RNA (crRNA) to find a matching viral sequence. Together, the crRNA and trans-activating crRNA (tracrRNA) guide CRISPR-associated (Cas) nuclease to cleave double-strand breaks in “foreign” DNA sequences. The prokaryotic CRISPR “immune system” has been engineered to function as an RNA-guided, mammalian genome editing tool that is simple, easy and quick to implement. STAR (which includes synthetic crRNA and tracrRNA) when combined with Cas12a protein can form ribonucleoprotein (RNP) complexes that target a specific genomic locus. Engineered guide nucleic acids formed with the RNA (STAR) system can result in a split gRNA. Split gRNA, i.e., dual guide RNAs are described more fully in WO 2021067788A1.

In certain embodiments, provided herein are ribonucleoprotein (RNP) complexes that include at least one nuclease disclosed herein. In certain embodiments, a RNP complex can include at least one nuclease having an amino acid sequence of at least 50% identity to SEQ ID NO: 2. In certain embodiments, a RNP complex can include at least one nuclease having an amino acid sequence of at least about 60%, 65%, 75%, 85%, 95%, 99% or about 100% identity to amino acid sequences of SEQ ID NO:2. In certain embodiments, a RNP complex can include at least one nuclease having an amino acid sequence of at least 50% identity to SEQ ID NO:3. In certain embodiments, a RNP complex can include at least one nuclease having an amino acid sequence of at least about 60%, 65%, 75%, 85%, 95%, 99% or about 100% identity to amino acid sequences of SEQ ID NO:3. In certain embodiments, a RNP complex can include at least one nuclease having an amino acid sequence of at least 50% identity to SEQ ID NO:4. In certain embodiments, a RNP complex can include at least one nuclease having an amino acid sequence of at least about 60%, 65%, 75%, 85%, 95%, 99% or about 100% identity to amino acid sequences of SEQ ID NO:4. In certain embodiments, a RNP complex including a nuclease disclosed herein can further include at least one STAR gRNA (dual guide RNA). In certain embodiments, a RNP complex including a nuclease disclosed herein can further include at least one non-STAR gRNA (e.g., single guide RNA). In certain embodiments, a RNP complex including a nuclease disclosed herein can further include at least one polynucleotide. In certain embodiments, a polynucleotide included in a RNP complex disclosed herein can be greater than about 50 nucleotides in length. In certain embodiments, a polynucleotide included in a RNP complex disclosed herein can be about 50, to about 150, to about 500, to about 1000 nucleotides, or greater than 1000 nucleotides in length. In certain embodiments, more than one nuclease can be added to an RNP complex to affect the overall editing efficiency. In certain embodiments, more than one gRNA can be added to the RNP complex to allow for multiplexed editing of more than one site in a single transfection for improved efficiency. In other embodiments, more than one DNA template can be added to the RNP to allow for multiplexed editing at one or more sites based on a specific desired repair outcome.

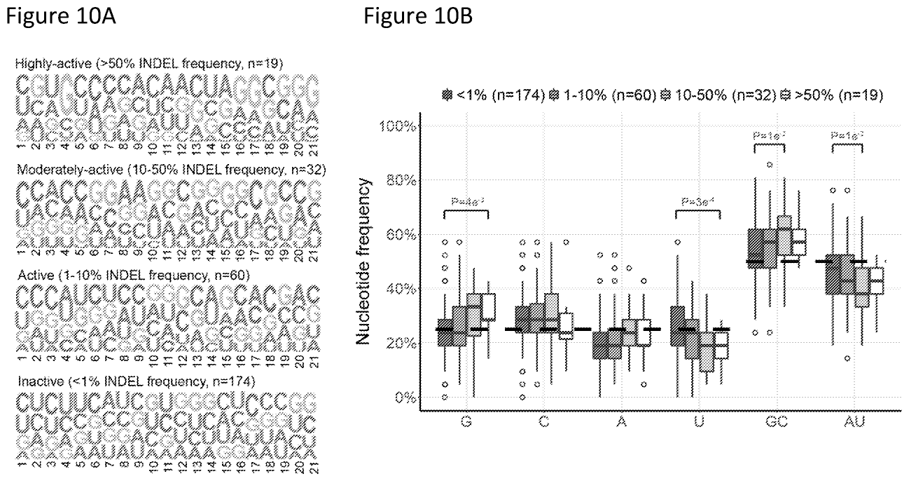

In certain embodiments, a composition comprising a Type V, e.g., Type VA, CRISPR nuclease polypeptide, such as described herein, further comprises a guide nucleic acid (gNA), e.g., gRNA, comprising a spacer sequence that targets a target nucleotide sequence (also referred to herein as a target nucleic acid sequence) within a polynucleotide (also referred to herein as a target polynucleotide, as will be clear from context), or a polynuclotide coding for the gNA, e.g., gRNA, wherein the gNA, e.g., gRNA is compatible with the Type V, e.g., Type VA, CRISPR nuclease. In general, a polynucleotide within which a target target nucleotide sequence (target nucleic acid sequence) is located, as that term is used herein, includes a polynucleotide that includes the target target nucleotide sequence (target nucleic acid sequence). Such a polynucleotide can be any suitable polynucleotide, such as a genome of a cell or part of a genome of a cell. In certain embodiments, the target nucleotide sequence (target nucleic acid sequence) is within 50 nucleotides of a protospacer adjacent motif (PAM) sequence specific for the Type V CRISPR nuclease, such as a PAM comprising a sequence of YTTN, wherein Y is T or C and Nis A, T, G, or C, or a sequence of YTTV or TTTV, wherein Vis A, G, or C. In certain embodiments the PAM comprises a sequence of YTTV or TTTV, wherein Vis A, G, or C. In certain embodiments, the gNA is a gRNA, such as a dual (split) gRNA. The gNA, e.g. gRNA, can comprise one or more chemical modifications, such as 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In certain embodiments, a ratio of guanine:uracil in the gRNA is at least 51:49, 52:48, 53:47, 54:46, 55:45, 56:44, 57:43, 58:42, 59:42, or 60:40, preferably at least 53:47, more preferably at least 54:46, even more preferably at least 55:45. See Example 12 and FIG. 10. In certain embodiments, a molar ratio of gNA, e.g., gRNA to Type V CRISPR nuclease is at least 1.1:1, 1.2:1, 1.3:1, 1.4:1, 1.5:1, 1.6:1, 1.7:1, 1.8:1, 2:1, 2.2:1, 2.5:1, or 3:1 and/or not more than 1.2:1, 1.3:1, 1.4:1, 1.5:1, 1.6:1, 1.7:1, 1.8:1, 2:1, 2.2:1, 2.5:1, 3:1, or 4:1, preferably 1.1:1 to 2.5:1, more preferably 1.2:1 to 2:1, even more preferably 1.2:1 to 1.7:1. See, e.g., Example 13. In certain embodiments a molar amount of gNA, e.g., gRNA, is at least 10, 25, 30, 35, 40, 45, 50, 55, 60, 65, 70, 75, 80, 85, 90, 95, 100, 110, 120, 130, 140, 150, 170, 190 or 200 pmol and/or not more than 30, 35, 40, 45, 50, 55, 60, 65, 70, 75, 80, 85, 90, 95, 100, 110, 120, 130, 140, 150, 170, 190, 200, 250, or 300 pmol, preferably 25-200 pmol, more preferably 50-100 pmol, even more preferably 65 to 85 pmol. See Exmple 13.

In certain embodiments, a composition comprising a Type V, e.g., Type VA, CRISPR nuclease polypeptide, such as described herein, further includes a donor template, also referred to as an editing template herein. A donor template can comprise homology arms, that is, nucleotide sequences that are complementary with polynucleotide sequenes on either side of a cleavage site at which the donor template will be inserted. The donor template can be present in any suitable amount, e.g., in certain embodiments, at least 0.1, 0.2, 0.3, 0.4, 0.5, 0.6, 0.7, 0.8, 0.9, 1, 1.1, 1.2, 1.3, 1.4, 1.5, 1.7, 2, 2.5, 3, 4, or 5 μg μL⁻¹ and/or not more than 0.2, 0.3, 0.4, 0.5, 0.6, 0.7, 0.8, 0.9, 1, 1.1, 1.2, 1.3, 1.4, 1.5, 1.7, 2, 2.5, 3, 4, 5, 7, or 10 μg μL⁻¹, preferably 0.3 to 2 μg μL⁻¹, more preferably 0.5 to 1.5 μg μL⁻¹, even more preferably 0.8 to 1.2 μg μL⁻¹.

In certain embodiments, a composition comprising a Type V, e.g., Type VA, CRISPR nuclease polypeptide, such as described herein, further includes an anionic polymer. Any suitable anionic polymer may be used. Exemplary anionic polymers include 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurea, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl) phosphine (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In certain embodiments, an anionic polymer comprises polyglutamic acid. In certain embodiments, the anionic polymer, e.g., PGA, is present at a concentration of at least 20, 50, 60, 70, 80, 90, 100, 110, 120, 130, 140, 150, 170, 200, 250, 300, 400, or 500 μg μL⁻¹ and/or not more than 50, 60, 70, 80, 90, 100, 110, 120, 130, 140, 150, 170, 200, 250, 300, 400, 500, 700, or 1000 μg μL⁻¹, preferably 20 to 200 μg μL⁻¹, more preferably 50 to 150 μg μL⁻¹, even more preferably 80 to 120 μg μL⁻¹. (PGA).

In certain embodiments, provided herein is a cell containing one or more of the compositions described herein, e.g. a composition comprising a Type V, e.g., Type VA, CRISPR nuclease polypeptide comprising one or more NLSs and, in certain embodiments a purification tag and/or cleavage site. Any suitable cell may be used. In certain embodiments the cell is a human cell, such as an immune cell, e.g., T cell, or a stem cell, e.g., induced pluripotent stem cell (iPSC).

In certain embodiments, provided herein are methods of inserting one or more of the compositions described herein, e.g., a composition comprising a Type V, e.g., Type VA, CRISPR nuclease polypeptide comprising one or more NLSs and, in certain embodiments a purification tag and/or cleavage site, into a cell. Any suitable method for insertion may be used. In certain embodiments, electroporation is used. Electroporation conditions can be optimized, see, e.g., Examples.

In certain embodiments provided are methods of modifying a target polynucleotide comprising contacting the target polynucleotide with a composition or compositions as described herein, e.g, a composition comprising a Type V, e.g., Type VA, CRISPR nuclease polypeptide comprising one or more NLSs and a suitable gNA, e.g., gRNA, and allowing the composition to modify the target polynucleotide, in some cases a genomic region, such as a genome or part of a genome within a cell, e.g. human cell such as an immune cell, e.g., T cell, or a stem cell, e.g., iPSC. In certain cases, the composition or compositions comprises a donor template, such as a donor template comprising a polynucleotide coding for a polypeptide to be expressed by the cell, in certain embodiments the polypeptide comprises a chimeric antigen receptor (CAR) or portion thereof; see, e.g., Examples. In certain embodiments the cell is a human cell, e.g., immune cell such as a T cell, or stem cell, such as an iPSC.

### Nuclease Systems

In certain embodiments disclosed herein are targetable nuclease systems. In certain embodiments, targetable nuclease system can include a nucleic acid-guided nuclease and a compatible guide nucleic acid (also referred to interchangeably herein as “guide polynucleotide” and “gRNA”). A targetable nuclease system can include a nucleic acid-guided nuclease or a polynucleotide sequence encoding the nucleic acid-guided nuclease. A targetable nuclease system can include a guide nucleic acid or a polynucleotide sequence encoding the guide nucleic acid.

In general, a targetable nuclease system as disclosed herein can be characterized by elements that promote the formation of a targetable nuclease complex at the site of a target sequence, wherein the targetable nuclease complex includes a nucleic acid-guided nuclease and a guide nucleic acid.

A guide nucleic acid together with a nucleic acid-guided nuclease forms a targetable nuclease complex which is capable of binding to a target sequence within a target polynucleotide, as determined by the guide sequence of the guide nucleic acid.

In general, to generate a double stranded break, in most cases a targetable nuclease complex binds to a target sequence as determined by the guide nucleic acid, and the nuclease has to recognize a protospacer adjacent motif (PAM) sequence adjacent to the target sequence.

A targetable nuclease complex can include a nucleic acid-guided nuclease having an amino acid sequence of at least 50% identity to SEQ ID NO: 2 and a compatible guide nucleic acid. A targetable nuclease complex can include a nucleic acid-guided nuclease having an amino acid sequence of at least about 60%, 65%, 75%, 85%, 95%, 99% or about 100% identity to amino acid sequences SEQ ID NO: 2 and a compatible guide nucleic acid. protospacer adjacent motif (PAM) sequence adjacent to the target sequence. A targetable nuclease complex can include a nucleic acid-guided nuclease having an amino acid sequence of at least 50% identity to SEQ ID NO: 3 and a compatible guide nucleic acid. A targetable nuclease complex can include a nucleic acid-guided nuclease having an amino acid sequence of at least about 60%, 65%, 75%, 85%, 95%, 99% or about 100% identity to amino acid sequences of SEQ ID NO: 3 and a compatible guide nucleic acid. A targetable nuclease complex can include a nucleic acid-guided nuclease having an amino acid sequence of at least 50% identity to SEQ ID NO: 4 and a compatible guide nucleic acid. A targetable nuclease complex can include a nucleic acid-guided nuclease having an amino acid sequence of at least about 60%, 65%, 75%, 85%, 95%, 99% or about 100% identity to amino acid sequences of SEQ ID NO: 4 and a compatible guide nucleic acid. In certain embodiments, the guide nucleic acid can include a scaffold sequence compatible with the nucleic acid-guided nuclease selected. In any of these embodiments, the guide sequence can be engineered to be complementary to any desired target sequence. The guide sequence selected can be engineered to hybridize to any desired target sequence. In certain embodiments, the guide sequence is a dual guide RNA.

A target sequence of a targetable nuclease complex can be any polynucleotide endogenous or exogenous to a prokaryotic or eukaryotic cell, or in vitro. For example, the target sequence can be a polynucleotide residing in the nucleus of the eukaryotic cell. A target sequence can be a sequence coding a gene product (e.g., a protein) or a non-coding sequence (e.g., a regulatory polynucleotide or a junk DNA). It is contemplated herein that the target sequence should be associated with a PAM; that is, a short sequence recognized by a targetable nuclease complex. The precise sequence and length requirements for a PAM differ depending on the nucleic acid-guided nuclease used, but PAMs can be a 2-5 base pair sequences adjacent the target sequence. Examples of PAM sequences are given in the examples section below, and the skilled person will be able to identify further PAM sequences for use with a given nucleic acid-guided nuclease. Further, engineering of the PAM Interacting (PI) domain may allow programming of PAM specificity, improve target site recognition fidelity, and increase the versatility of a nucleic acid-guided nuclease genome engineering platform. Nucleic acid-guided nucleases may be engineered to alter their PAM specificity, for example as described in Kleinstiver et al., Nature. 2015 Jul. 23; 523 (7561): 481-5, the disclosure of which is incorporated herein in its entirety.

A PAM site is a nucleotide sequence in proximity to a target sequence. In most cases, a nucleic acid-guided nuclease can only cleave a target sequence if an appropriate PAM is present. PAMs are nucleic acid-guided nuclease-specific and can be different between two different nucleic acid-guided nucleases. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments disclosed herein, a PAM can be provided on a separate oligonucleotide. In such cases, providing PAM on a oligonucleotide allows cleavage of a target sequence that otherwise would not be able to be cleave because no adjacent PAM is present on the same polynucleotide as the target sequence.

Polynucleotide sequences encoding a component of a targetable nuclease system can include one or more vectors. In general, the term “vector” as used herein can refer to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Other vectors (e.g., non-episomal mammalian vectors) can be integrated into the genome of a host cell upon introduction into the host cell. Recombinant expression vectors can include a nucleic acid of the invention in a form suitable for expression of the nucleic acid in a host cell, can mean that the recombinant expression vectors include one or more regulatory elements, which may be selected on the basis of the host cells to be used for expression, that is operatively-linked to the nucleic acid sequence to be expressed.

In some embodiments, a regulatory element can be operably linked to one or more elements of a targetable nuclease system so as to drive expression of the one or more components of the targetable nuclease system.

In some embodiments, a vector can include a regulatory element operably linked to a polynucleotide sequence encoding a nucleic acid-guided nuclease. The polynucleotide sequence encoding the nucleic acid-guided nuclease can be codon optimized for expression in targeted cells, such as prokaryotic or eukaryotic cells. Eukaryotic cells can be yeast, fungi, algae, plant, animal, or human cells. Eukaryotic cells can be those derived from an organism, such as a mammal, including but not limited to human, mouse, rat, rabbit, dog, or non-human mammal including non-human primate.

In general, codon optimization can refer to a process of modifying a nucleic acid sequence for enhanced expression in the host cells of interest by replacing at least one codon or more of the native sequence with codons that are more frequently or most frequently used in the genes of that host cell while maintaining the native amino acid sequence. Various species exhibit certain bias for codons of a certain amino acid. As contemplated herein, genes can be tailored for optimal gene expression in a given organism based on codon optimization. Codon usage tables are readily available, for example, at the “Codon Usage Database” available at www.kazusa.orjp and these tables can be adapted in a number of ways. See Nakamura, Y., et al. “Codon usage tabulated from the international DNA sequence databases: status for the year 2000” Nucl. Acids Res. 28:292 (2000).

A nucleic acid-guided nuclease and one or more guide nucleic acids can be delivered either as DNA or RNA. Delivery of a nucleic acid-guided nuclease and guide nucleic acid both as RNA (unmodified or containing base or backbone modifications) molecules can be used to reduce the amount of time that the nucleic acid-guided nuclease persists in the cell. This may reduce the level of off-target cleavage activity in the target cell. Since a nucleic acid-guided nuclease as mRNA takes time to be translated into protein, it can be advantageous to deliver the guide nucleic acid several hours following the delivery of the nucleic acid-guided nuclease mRNA, to maximize the level of guide nucleic acid available for interaction with the nucleic acid-guided nuclease protein. In other cases, the nucleic acid-guided nuclease mRNA and guide nucleic acid are delivered concomitantly. In other examples, the guide nucleic acid is delivered sequentially, such as 0.5, 1, 2, 3, 4, or more hours after the nucleic acid-guided nuclease mRNA.

Guide nucleic acid in the form of RNA or encoded on a DNA expression cassette can be introduced into a host cell can include a nucleic acid-guided nuclease encoded on a vector or chromosome. The guide nucleic acid may be provided in the cassette one or more polynucleotides, which may be contiguous or non-contiguous in the cassette. In specific embodiments, the guide nucleic acid is provided in the cassette as a single contiguous polynucleotide.

A variety of delivery systems can be used to introduce a nucleic acid-guided nuclease (DNA or RNA) and guide nucleic acid (DNA or RNA) into a host cell. In accordance with these embodiments, systems of use can include, but are not limited to, yeast systems, lipofection systems, microinjection systems, biolistic systems, virosomes, liposomes, immunoliposomes, polycations, lipid: nucleic acid conjugates, virions, artificial virions, viral vectors, electroporation, cell permeable peptides, nanoparticles, nanowires (Shalek et al., Nano Letters, 2012), exosomes. Molecular trojan horses liposomes (Pardridge et al., Cold Spring Harb Protoc; 2010; doi: 10.1101/pdb.prot5407) may be used to deliver an engineered nuclease and guide nuclease across the blood brain barrier.

In some embodiments, an editing template, also referred to herein as a donor template, is also provided. An editing template may be a component of a vector as described herein, contained in a separate vector, or provided as a separate polynucleotide, such as an oligonucleotide, linear polynucleotide, or synthetic polynucleotide. In some cases, an editing template is on the same polynucleotide as a guide nucleic acid. In some embodiments, an editing template is designed to serve as a template in homologous recombination, such as within or near a target sequence nicked or cleaved by a nucleic acid-guided nuclease as a part of a complex as disclosed herein. An editing template polynucleotide can be of any suitable length, such as about or more than about 10, 15, 20, 25, 50, 75, 100, 150, 200, 500, 1000, or more nucleotides in length. In some embodiments, the editing template polynucleotide is complementary to a portion of a polynucleotide can include the target sequence. When optimally aligned, an editing template polynucleotide might overlap with one or more nucleotides of a target sequences (e.g. about or more than about 1, 5, 10, 15, 20, 25, 30, 35, 40, or more nucleotides). In some embodiments, when a editing template sequence and a polynucleotide can include a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In some embodiments, methods are provided for delivering one or more polynucleotides, such as or one or more vectors or linear polynucleotides as described herein, one or more transcripts thereof, and/or one or proteins transcribed therefrom, to a host cell. In some aspects, the invention further provides cells produced by such methods, and organisms can include or produced from such cells. In some embodiments, an engineered nuclease in combination with (and optionally complexed with) a guide nucleic acid is delivered to a cell.

Conventional viral and non-viral based gene transfer methods can be used to introduce nucleic acids in cells, such as prokaryotic cells, eukaryotic cells, mammalian cells, or target tissues. Such methods can be used to administer nucleic acids encoding components of an engineered nucleic acid-guided nuclease system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Any gene therapy method known in the art is contemplated of use herein. Methods of non-viral delivery of nucleic acids include are contemplated herein. Adeno-associated virus (“AAV”) vectors may also be used to transduce cells with target nucleic acids, e.g., in the in vitro production of nucleic acids and peptides, and for in vivo and ex vivo gene therapy procedures.

In some embodiments, a host cell is transiently or non-transiently transfected with one or more vectors, linear polynucleotides, polypeptides, nucleic acid-protein complexes, or any combination thereof as described herein. In some embodiments, a cell in transfected in vitro, in culture, or ex vivo. In some embodiments, a cell is transfected as it naturally occurs in a subject. In some embodiments, a cell that is transfected is taken from a subject. In some embodiments, the cell is derived from cells taken from a subject, such as a cell line.

In some embodiments, a cell transfected with one or more vectors, linear polynucleotides, polypeptides, nucleic acid-protein complexes, or any combination thereof as described herein is used to establish a new cell line can include one or more transfection-derived sequences. In some embodiments, a cell transiently transfected with the components of an engineered nucleic acid-guided nuclease system as described herein (such as by transient transfection of one or more vectors, or transfection with RNA), and modified through the activity of an engineered nuclease complex, is used to establish a new cell line can include cells containing the modification but lacking any other exogenous sequence.

In some embodiments, one or more vectors described herein are used to produce a non-human transgenic cell, organism, animal, or plant. In some embodiments, the transgenic animal is a mammal, such as a mouse, rat, or rabbit. Methods for producing transgenic cells, organisms, plants, and animals are known in the art, and generally begin with a method of cell transformation or transfection, such as described herein.

In certain embodiments, an engineered nuclease complex, “target sequence” can refer to a sequence to which a guide sequence is designed to have complementarity, where hybridization between a target sequence and a guide sequence promotes the formation of an engineered nuclease complex. A target sequence can include any polynucleotide, such as DNA, RNA, or a DNA-RNA hybrid. A target sequence can be located in the nucleus or cytoplasm of a cell. A target sequence can be located in vitro or in a cell-free environment.

In some embodiments, formation of an engineered nuclease complex can include a guide nucleic acid hybridized to a target sequence and complexed with one or more novel engineered nucleases as disclosed herein renders cleavage of one or both strands in or near (e.g. within 1, 2, 3, 4, 5, 6, 7, 8, 9, 10, 20, 30, 40, 50, or more base pairs from) the targeted sequence. Cleavage can occur within a target sequence, 5′ of the target sequence, upstream of a target sequence, 3′ of the target sequence, or downstream of a target sequence.

In some embodiments, one or more vectors driving expression of one or more components of a targetable nuclease system are introduced into a host cell or in vitro such formation of a targetable nuclease complex at one or more target sites. For example, a nucleic acid-guided nuclease and a guide nucleic acid can each be operably linked to separate regulatory elements on separate vectors. Alternatively, two or more of the elements expressed from the same or different regulatory elements, can be combined in a single vector, with one or more additional vectors providing any components of the targetable nuclease system not included in the first vector. Targetable nuclease system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a nucleic acid-guided nuclease and one or more guide nucleic acids. In some embodiments, a nucleic acid-guided nuclease and one or more guide nucleic acids are operably linked to and expressed from the same promoter. In other embodiments, one or more guide nucleic acids or polynucleotides encoding the one or more guide nucleic acids are introduced into a cell or in vitro environment already can include a nucleic acid-guided nuclease or polynucleotide sequence encoding the nucleic acid-guided nuclease.

In some embodiments, when multiple different guide sequences are used, a single expression construct may be used to target nuclease activity to multiple different, corresponding target sequences within a cell or in vitro. For example, a single vector can include about or more than about 1, 2, 3, 4, 5, 6, 7, 8, 9, 10, 15, 20, or more guide sequences. In other embodiments, about or more than about 1, 2, 3, 4, 5, 6, 7, 8, 9, 10, or more such guide-sequence-containing vectors can be provided, and optionally, delivered to a cell in vivo or in vitro.

In some embodiments, methods and compositions disclosed herein can include more than one guide nucleic acid, such that each guide nucleic acid has a different guide sequence, thereby targeting a different target sequence. In accordance with these embodiments, multiple guide nucleic acids can be using in multiplexing, wherein multiple targets are targeted simultaneously. Additionally or alternatively, the multiple guide nucleic acids are introduced into a population of cells, such that each cell in a population received a different or random guide nucleic acid, thereby targeting multiple different target sequences across a population of cells. In such cases, the collection of subsequently altered cells can be referred to as a library.

In other embodiments, methods and compositions disclosed herein can include multiple different nucleic acid-guided nucleases, each with one or more different corresponding guide nucleic acids, thereby allowing targeting of different target sequences by different nucleic acid-guided nucleases. In some such cases, each nucleic acid-guided nuclease can correspond to a distinct plurality of guide nucleic acids, allowing two or more non-overlapping, partially overlapping, or completely overlapping multiplexing events.

In some embodiments, the nucleic acid-guided nuclease has DNA cleavage activity or RNA cleavage activity. In some embodiments, the nucleic acid-guided nuclease directs cleavage of one or both strands at the location of a target sequence, such as within the target sequence and/or within the complement of the target sequence. In some embodiments, the nucleic acid-guided nuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In certain embodiments, the invention provides for methods of modifying a target sequence in vitro, or in a prokaryotic or eukaryotic cell, which can be in vivo, ex vivo, or in vitro. In some embodiments, the method includes sampling a cell or population of cells such as prokaryotic cells, or those from a human or non-human animal or plant (including micro-algae or other organism), and modifying the cell or cells. Culturing may occur at any stage in vitro or ex vivo. The cell or cells may even be re-introduced into the host, such as a non-human animal or plant (including micro-algae). For re-introduced cells, they can be stem cells.

In some embodiments, the method includes allowing a targetable nuclease complex to bind to the target sequence to effect cleavage of the target sequence, thereby modifying the target sequence, wherein the targetable nuclease complex includes a nucleic acid-guided nuclease complexed with a guide nucleic acid wherein the guide sequence of the guide nucleic acid is hybridized to a target sequence within a target polynucleotide. In some aspects, the invention provides a method of modifying expression of a target polynucleotide in in vitro or in a prokaryotic or eukaryotic cell. In some embodiments, the method includes allowing an targetable nuclease complex to bind to a target sequence with the target polynucleotide such that the binding can lead to in increased or decreased expression of the target polynucleotide; wherein the targetable nuclease complex includes an nucleic acid-guided nuclease complexed with a guide nucleic acid, and wherein the guide sequence of the guide nucleic acid is hybridized to a target sequence within the target polynucleotide.

In certain embodiments, the invention provides kits containing any one or more of the elements disclosed in the above methods and compositions. Elements may provide individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents can be provided in a form that is usable in an assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit includes one or more oligonucleotides corresponding to a guide sequence for insertion into a vector so as to operably link the guide sequence and a regulatory element. In some embodiments, the kit includes a editing template.

In some embodiments, a targetable nuclease complex has a wide variety of utility including modifying (e.g., deleting, inserting, translocating, inactivating, activating) a target sequence in a multiplicity of cell types. As such a targetable nuclease complex of the invention has a broad spectrum of applications in, e.g., biochemical pathway optimization, genome-wide studies, genome engineering, gene therapy, drug screening, disease diagnosis, and prognosis. An exemplary targetable nuclease complex includes a nucleic acid-guided nuclease as disclosed herein complexed with a guide nucleic acid, wherein the guide sequence of the guide nucleic acid can hybridize to a target sequence within the target polynucleotide. A guide nucleic acid can include a guide sequence linked to a scaffold sequence. A scaffold sequence can include one or more sequence regions with a degree of complementarity such that together they form a secondary structure.

An editing template polynucleotide can include a sequence to be integrated (e.g., a mutated gene). A sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function. Sequence to be integrated may be a mutated or variant of an endogenous wild-type sequence. Alternatively, sequence to be integrated may be a wild-type version of an endogenous mutated sequence. Additionally or alternatively, sequenced to be integrated may be a variant or mutated form of an endogenous mutated or variant sequence.

In certain embodiments, an upstream or downstream sequence can include from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or about 2500 bp. In some embodiments, an exemplary upstream or downstream sequence has about 15 bp to about 2000 bp, about 30 bp to about 1000 bp, about 50 bp to about 750 bp, about 600 bp to about 1000 bp, or about 700 bp to about 1000 bp.

In some embodiments, the editing template polynucleotide can further include a marker. In certain embodiments, some markers can facilitate screening for targeted integrations. Examples of suitable markers can include, but are not limited to, restriction sites, fluorescent proteins, or selectable markers. In certain embodiments, an exogenous polynucleotide template can be constructed using recombinant techniques.

In one embodiment, an exemplary method for modifying a target polynucleotide by integrating an editing template polynucleotide, a double stranded break is introduced into the genome sequence by an engineered nuclease complex, the break can be repaired via homologous recombination using an editing template such that the template is integrated into the target polynucleotide. The presence of a double-stranded break can increase the efficiency of integration of the editing template.

Disclosed herein are methods for modifying expression of a polynucleotide in a cell. Some methods include increasing or decreasing expression of a target polynucleotide by using a targetable nuclease complex that binds to the target polynucleotide.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules can be proportional to the amount of the amplified DNA products, one can conveniently determine the amount of the amplified products by quantifying the fluorescence of the intercalated dye using conventional optical systems in the art. DNA-binding dye suitable for this application include, but are not limited to, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and others known by one of skill in the art.

In some embodiments, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan™ probes) resulting in increased specificity and sensitivity. Methods for performing probe-based quantitative amplification are well established in the art.

In some embodiments, an agent-induced change in expression of sequences associated with a signaling biochemical pathway can also be determined by examining the corresponding gene products. Determining the protein level can involve a) contacting the protein contained in a biological sample with an agent that specifically bind to a protein associated with a signaling biochemical pathway; and (b) identifying any agent: protein complex so formed. In one aspect of this embodiment, the agent that specifically binds a protein associated with a signaling biochemical pathway is an antibody, preferably a monoclonal antibody.

In some embodiments, the amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

In some embodiments, a number of techniques for protein analysis based on the general principles outlined above are known in the art and contemplated herein. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

In some embodiments, in practicing a subject method, it may be desirable to discern the expression pattern of a protein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

In other embodiment, an altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays.

In certain embodiments, where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a millisecond.

In practicing any of the methods disclosed herein, a suitable vector can be introduced to a cell, tissue, organism, or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

A target polynucleotide of a targetable nuclease complex can be any polynucleotide endogenous or exogenous to the host cell. For example, the target polynucleotide can be a polynucleotide residing in the nucleus of the eukaryotic cell, the genome of a prokaryotic cell, or an extrachromosomal vector of a host cell. The target polynucleotide can be a sequence coding a gene product (e.g., a protein) or a non-coding sequence (e.g., a regulatory polynucleotide or a junk DNA).

Some embodiments disclosed herein relate to use of an engineered nucleic acid guided nuclease system disclosed herein; for example, in order to target and knock out genes, amplify genes and/or repair certain mutations associated with DNA repeat instability and a medical disorder. This nuclease system may be used to harness and to correct these defects of genomic instability. In other embodiments, engineered nucleic acid guided nuclease systems disclosed herein can be used for correcting defects in the genes associated with Lafora disease. Lafora disease is an autosomal recessive condition which is characterized by progressive myoclonus epilepsy which may start as epileptic seizures in adolescence. This condition causes seizures, muscle spasms, difficulty walking, dementia, and eventually death.

In yet another aspect of the invention, the engineered/novel nucleic acid guided nuclease system can be used to correct genetic-eye disorders that arise from several genetic mutations

In certain embodiments disclosed herein engineered nucleic acid guided nuclease constructs can recognize a protospacer adjacent motif (PAM) sequence other than TTTN or in addition to TTTN. In other embodiments, engineered nucleic acid guided nuclease constructs disclosed herein can be further mutated to improve targeting efficiency or can be selected from a library for certain targeted features. Other embodiments disclosed herein concern vectors including constructs disclosed herein of use for further analysis and to select for improved genome editing features.

Other embodiments disclosed herein include kits for packaging and transporting nucleic acid guided nuclease constructs and/or novel gRNAs disclosed herein or known gRNAs disclosed herein and further include at least one container. In certain embodiments, several reagents required for the kits can be included for convenience and ease of transport and efficiency.

### EXAMPLES

### Example 1: Culture of Jurkat Human T-Cell Leukemia Cell Line and Primary Human T-Cells

Human Jurkat T-cell leukemia cells (Leibniz Institute DSMZ-German Collection of Microorganisms and Cell Cultures GmbH (ACC 282)) were propagated in RPMI 1640 medium (ThermoFisher Scientific) with 10% heat-inactivated fetal bovine serum (FBS) (ThermoFisher Scientific) supplemented with 1% penicillin-streptomycin antibiotic mix (ThermoFisher Scientific). Cells were cultured at 37° C. in 5% CO2 incubators and maintained at a density of 0.5 to 1.5×10⁶ cells mL⁻¹. 24 hours before transfection, cells were passaged at 0.1×10⁶ cell mL⁻¹. Cell culture media supernatant was periodically tested for mycoplasma contamination using the MycoAlert PLUS mycoplasma detection kit (Lonza).

### Example 2: Primary T-Cell Isolation and Culture

T-cells were isolated from human peripheral blood obtained from healthy adults by immune-magnetic negative selection using the EasySep Human T-cell Isolation Kit (STEMCELL Technologies). After isolation, T-cells were activated in 25 μL mL⁻¹ ImmunoCult Human CD3/CD28/CD2 T-Cell Activator (STEMCELL Technologies) in ImmunoCult-XF T-Cell Expansion Medium (STEMCELL Technologies) containing 12.5 ng mL⁻¹ Human Recombinant IL-2, 5 ng mL⁻¹ IL-7, and 5 ng mL⁻¹ IL-15 (STEMCELL Technologies) and seeded at 1.0×10⁶ cells mL-1. Until transfection 48 hours later, the cells were cultured at 37° C. in 5% CO2 incubators.

### Example 3: RNP Formulation

Ribonucleoprotein complexes (RNPs) were generated by incubating respective guide nucleic acids (gNAs) with MAD7 in the molar ratio of 3:2 gNA:MAD7 for 15 minutes at room temperature immediately before transfection. For Jurkat experiments, the RNP complexes were generated by mixing the respective gNA (150 μmol), MAD7 (100 μmol), and nuclease-free water, unless otherwise stated. For T-cell experiments, 1.6 μL of an aqueous solution of 15-50 kDa poly-L-glutamic acid (PGA, 100 μg μL⁻¹, Alamanda Polymers) was added to gNAs, followed by the addition of MAD7 and nuclease-free water.

### Example 4: Generation of Donor Templates Via PCT Amplification

Donor templates comprising site-specific homology arms, respective promoter, and respective gene (GFP or Hu19 scFv-CD8a-CD28-CD3ζ CAR) were amplified from corresponding pTwist Ampicillin high-copy plasmids (Twist Bioscience) using homology arms-specific PCR primers. Donor templates were amplified in a two-step PCR program: initial denaturation at 98° C. for 30 seconds, cycle denaturation at 98° C. for 10 seconds, extension at 72° C. for 30 seconds per kb amplicon for 40-cycles with a hold at 72° C. for 10 minutes. Each 50 μL PCR reaction contained 10 ng amplification template (plasmid DNA), 0.5 μM homology arm-specific forward and reverse primers, nuclease-free water (IDT), 3% DMSO, and 1×Phusion High-Fidelity PCR Master Mix with HF Buffer (ThermoFisher Scientific). PCR products were purified using NucleoSpin Gel and PCR Clean-up Kit (Macherey-Nagel) with two 20 μL elutions. Purified HDR templates were collected and quantified on NanoDrop One Microvolume UV-Vis Spectrophotometer (ThermoFisher Scientific). Templates were concentrated using Amicon Ultra 0.5 mL 30K Centrifugal Filters: 100 μg DNA per unit was transferred, filled with nuclease-free water to 500 μL, and centrifuged at 10,000 g for 10 minutes to reduce volume to 50 μL. DNA was washed twice with nuclease-free water and recovered into a fresh tube by inversion and centrifugation at 10,000 g for 15 seconds. HDR templates were collected, diluted, and concentrations quantified using Qubit dsDNA HS Assay Kit (ThermoFisher Scientific). HDR templates of 0.5 to 1 μg μL⁻¹ were used for cellular studies.

### Example 5: Jurkat Cell Transfection

Lonza 4D Nucleofector with Shuttle unit (V4SC-2960 Nucleocuvette Strips) was used for transfection, following the manufacturer's instructions. For transfection, cells were harvested by centrifugation (200 g, RT, 5 minutes) and re-suspended in 20 μL at 10×10⁶ cells mL⁻¹ in the SF Cell Line Nucleofector X Kit buffer (Lonza), unless stated otherwise. The cell suspension was mixed with the RNPs, immediately transferred to the nucleocuvette, and transfected. After transfection, the cells were immediately re-suspended in the pre-warmed cultivation medium and plated onto 96-well, flat-bottom, non-cell culture treated plates (Falcon), and cultured at 37° C. in 5% CO₂ incubators and maintained at a density of 0.5 to 1.0×10⁶ cells mL⁻¹. After 48 hours, the cells were harvested for the viability assay and genomic DNA, as described below. For the Homology-Directed Repair Template insertion, the HDR template was added to the cells and the suspension transferred to the RNPs immediately before transfection. The transfection parameters, cell recovery step, and proliferation conditions as described in Example 1. The cells were harvested 48 hours post-transfection for the viability assessment, after 7 days for CAR insertion efficiency, or after 7 days, 14 days, and 21 days for GFP insertion efficiency.

### Example 6: Primary T-Cell Transfection

48 hours after isolation, the cells were harvested by centrifugation (300 g, RT, 5 minutes) and re-suspended in 20 μL at 50×10⁶ cells mL⁻¹ in the supplemented P3 Primary Cell Nucleofector Kit buffer (Lonza). The cells were mixed with HDR templates and the suspension transferred to the RNPs immediately before transfection (Nucleofection program EH-115). After transfection, 80 μL of pre-warmed cultivation medium without IL-2 was added to the electroporation cuvettes. When using M3814 (Selleckchem), 80 μL of pre-warmed cultivation medium containing 2 μM M3814 final concentration without IL-2 was added to the electroporation cuvettes. After 10 minutes of incubation at 37° C., T-cells were transferred onto 96-well, flat-bottom, non-cell culture treated plates (Falcon) containing pre-warmed cultivation medium pretreated with 2 μM M3814 final concentration and 12.5 ng mL⁻¹ IL-2. The cells were seeded at a density of 0.25×10⁶ cells mL-1, or 1.3×10⁶ cells mL⁻¹ in the experiment with M3814, and kept at 37° C. in 5% CO₂ incubators. The viability assay was carried out 24 hours post-transfection after which the cells were reseeded in the fresh cultivation medium containing IL-2. Insertion efficiency of CAR was measured after 7 days, and 11 days or 13 days post-transfection.

### Example 7: Flow Cytometry

Flow cytometric assessments were carried out on a CytoFLEX S instrument (Beckmen Coulter) using a 96-well plate format. Measurements of cell viability, PDCD1 expression, GFP expression, and CAR expression were performed on 10,000 or 20,000 single cell events in Jurkat or primary T-cells, respectively.

For the cell viability and GFP knock-in measurements, approximately 250,000 cells per sample were transferred onto 96-well V-bottom cell culture plates and assessed following a series of consecutive washing and staining steps. The first step included centrifuging the cells at 300 g for 5 minutes at room temperature, discarding the supernatant, and washing cells in 150 μL Dulbecco's PBS/2% FBS (STEMCELL Technologies) or Cell Staining Buffer (Biolegend), respectively, followed by the second centrifugation and removal of supernatant. The final step included viability staining of cells using 150 μL Dulbecco's PBS/2% FBS with 7-amino-actinomycin D (7-AAD, 1:1,000; ThermoFisher) or 50 μL Cell Staining Buffer with Zombie Violet Dye (1:200; Biolegend), respectively. The measurements of cell viability and GFP expression were collected simultaneously for 7-AAD (excitation: yellow-green laser; emission: 561 nm), Zombie Violet (excitation: violet laser; emission 405 nm), and GFP (excitation: blue laser; emission 488 nm) as needed.

For detection of CAR knock-in efficiency, approx. 250,000 cells per sample were transferred onto 96-well V-bottom, washed as described above using Cell Staining Buffer, and re-suspended in 50 μL Cell Staining Buffer with PE Anti-Myc tag antibody [9E10] (1:50; Abcam) and Zombie Violet Dye (1:200; Biolegend) for 30 minutes. Afterwards, the cells were washed in two subsequent washing steps using 150 μL Cell Staining Buffer, and finally re-suspended in 100 μL Cell Staining Buffer for the flow cytometry measurements (excitation: yellow-green laser; emission: 561 nm).

For detection of PDCD1 knock-out efficiency, approx. 250,000 Jurkat cells per sample were transferred onto 96-well V-bottom cell culture plates and assessed following a series of consecutive washing and staining steps. The first step included centrifuging the cells at 300 g for 5 minutes at 4° C. and discarding the supernatant. Afterwards, the cells were stained using 100 μL Cell Staining Buffer (Biolegend) with APC/Cyanine7 anti-human CD279 (PD-1) antibody (1:100; Biolegend) and incubated for 30 minutes at 4° C. in the dark. The cells were then centrifuged at 300 g for 5 minutes at 4° C. and the supernatant discarded. The next step included two repeats of centrifugation at 300 g for 5 minutes at 4° C., supernatant removal, and cell washing in 150 μL ice-cold Cell Staining Buffer (Biolegend). In the final step, the cells were re-suspended in 100 μL Cell Staining Buffer for the flow cytometry measurements (excitation: red laser; emission: 633 nm).

### Example 8: DNA Extraction

Cells were harvested 48-h post-transfection by centrifugation (1,000 g, 10 minutes) in 96-well, V-bottom plates (Greiner), washed with PBS (Sigma Aldrich) and lysed in 20 μL QuickExtract DNA Extraction Solution (Epicentre, Lucigen). DNA was extracted following the manufacturer's protocol: 15 minutes at 65° C., 15 minutes at 68° C., 10 minutes at 95° C., cooled to 4° C., and stored at 4° C. Genomic DNA was diluted 20-fold in nuclease-free water before amplicon PCR reactions.

### Example 9: Amplicon Sequencing

Extracted genomic DNA was quantified using the NanoDrop (ThermoFisher Scientific). Amplicons were constructed in two PCR steps: in the first PCR, regions of interest (150-400 bp) were amplified from 10 to 30 ng of genomic DNA with primers containing Illumina forward and reverse adapters on both ends comprising suitable loci-specific complementary sequences, using Phusion High-Fidelity PCR Master Mix (ThermoFisher Scientific). Amplification products were purified with Agencourt AMPure XP beads (Ramcon), using the sample to beads ratio of 1:1.8. The DNA was eluted from the beads with nuclease-free water and the size of the purified amplicons analyzed on a 2% agarose E-gel using the E-gel electrophoresis system (ThermoFisher Scientific). In the second PCR, unique pairs of Illumina-compatible indexes (Nextera XT Index Kit v2) were added to the amplicons using the KAPA HiFi HotStart Ready Mix (Roche). The amplified products were purified with Agencourt AMPure XP beads (Ramcon), using the sample to bead ratio of 1:1.8. The DNA was eluted from the beads with 10 mM Tris-HCl pH 8.5, 0.1% Tween 20. Sizes of the purified DNA fragments were validated on a 2% agarose gel using the E-gel electrophoresis system (ThermoFisher Scientific), quantified using Qubit dsDNA HS Assay Kit (Thermo Fisher) and then pooled in equimolar concentrations. Quality of the amplicon library was validated using Bioanalyzer, High Sensitivity DNA Kit (Agilent) before sequencing. The final library was sequenced on Illumina MiSeq System using the MiSeq Reagent Kit v.2 (300 cycles, 2×250 bp, paired-end reads). De-multiplexed FASTQ files were obtained from BaseSpace (Illumina).

### Example 10: NGS Data Analysis

Initial quality assessment of the obtained reads was performed with FastQC36. The sequencing data were aligned and analyzed with the CRISPResso2 software, using CRISPRessoBatch command with the parameters --cleavage_offset 1--quantification_window_size 10 -- --quantification_window_center 1--expand_ambiguous_alignments for the INDEL frequency analysis. For the ORF disruption analysis, CRISPRessoBatch command with the parameters --cleavage_offset 1-coding_seq<EXON_SEQ>--quantification_window_size 0--quantification_window_center 1--expand_ambiguous_alignments was used. Modification rates from the CRISPResso2 software output were analyzed in Excel.

### Example 11: CRISPR-MAD7 Platform for Human Genome Editing Using the Jurkat T-Cell Leukemia Line

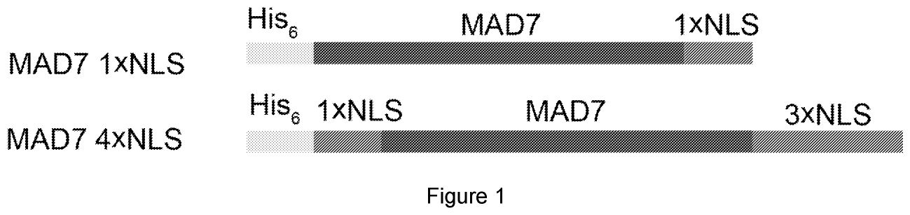

MAD7 nuclease comprising a His6 tag (SEQ ID NO: 423) and either one (MAD7-1NLS) or four (MAD7-4NLS) nuclear localization signals (NLS) were used (FIG. 1). RNPs were generated as described in Example 3. Editing frequency of the MAD7 nuclease complexed with one or more guide nucleic acids comprising a spacer sequence of SEQ ID NOs: 86-384 as shown in Table 1 was determined by nucleofection of RNPs in Jurkat T-cells using the Lonza recommended nucleofection program SE-CL-120 (Example 5), followed by genomic DNA extraction (Example 8), amplification of the edited locus and targeted next-generation sequencing (Example 9) for identification of the edits, and finally by computational analysis (Example 10) of modification frequency using the CRISPResso2 algorithm.

TABLE 1
Spacer sequences

| | | SEQ | |
|---|---|---|---|
| | | ID | |
| Name | PAM | NO | Spacer sequence |
| crCD247_1 | TTTC | 114 | ACCGCGGCCAUCCUGCAGGCA |
| crCD247_2 | TTTC | 115 | UGAGGGAAAGGACAAGAUGAA |
| crCD247_3 | TTTG | 116 | GGAUCCAGCAGGCCAAAGCUC |
| crCD247_4 | TTTC | 117 | CUAGCAGAGAAGGAAGAACCC |
| crCD247_5 | TTTC | 118 | UGUGUUGCAGUUCAGCAGGAG |
| crCD247_6 | CTTC | 119 | CUGAGGGUUCUUCCUUCUCUG |
| crCD247_7 | CTTC | 120 | CCGUUGUCUUUCCUAGCAGAG |
| crCD247_8 | TTTC | 121 | UGCAGUUCCUGCAGAAGAGGG |
| crCD247_9 | CTTC | 122 | UGCAGGAACUGCAGAAAGAUA |
| crCD247_10 | TTTC | 123 | AUCCCAAUCUCACUGUAGGCC |
| crCD247_11 | CTTT | 124 | CAUCCCAAUCUCACUGUAGGC |
| crCD247_12 | TTTT | 125 | CUCAUUUCACUCCCAAACAAC |
| crCD247_13 | TTTC | 126 | UCAUUUCACUCCCAAACAACC |
| crCD247_14 | TTTC | 127 | ACUCCCAAACAACCAGCGCCG |
| crCD247_15 | CTTA | 128 | CGUUAUAGAGCUGGUUCUGGC |
| crCD247_16 | TTTG | 129 | UUUUCUGAUUUGCUUUCACGC |
| crCD247_17 | TTTC | 130 | UGAUUUGCUUUCACGCCAGGG |
| crCD247_18 | TTTG | 131 | CUUUCACGCCAGGGUCUCAGU |
| crCD247_19 | TTTC | 132 | ACGCCAGGGUCUCAGUACAGC |
| crCD247_20 | TTTC | 133 | CGGAGGGUCUACGGCGAGGCU |
| crCD247_21 | TTTC | 134 | UUAUCUGUUAUAGGAGCUCAA |
| crCD247_22 | CTTA | 135 | UCUGUUAUAGGAGCUCAAUCU |
| crCD247_23 | CTTG | 136 | UCCAAAACAUCGUACUCCUCU |
| crCD247_24 | TTTC | 137 | CCCCCAUCUCAGGGUCCCGGC |
| crCD247_25 | TTTG | 138 | GACAAGAGACGUGGCCGGGAC |
| crCD247_26 | TTTC | 139 | UCUCCCUCUAACGUCUUCCCG |
| crCTLA4_1 | TTTG | 140 | CCUGGAGAUGCAUACUCACAC |
| crCTLA4_2 | TTTG | 141 | CAGAAGACAGGGAUGAAGAGA |
| crCTLA4_3 | TTTC | 142 | CACUGGAGGUGCCCGUGCAGA |
| crCTLA4_4 | TTTG | 143 | UGUGUGAGUAUGCAUCUCCAG |
| crCTLA4_5 | TTTC | 144 | AGCGGCACAAGGCUCAGCUGA |
| crCTLA4_6 | CTTG | 145 | UGCCGCUGAAAUCCAAGGCAA |
| crCTLA4_7 | CTTT | 146 | UCCAUGCUAGCAAUGCACGUG |
| crCTLA4_8 | TTTT | 147 | CCAUGCUAGCAAUGCACGUGG |
| crCTLA4_9 | CTTT | 148 | GUGUGUGAGUAUGCAUCUCCA |
| crCTLA4_10 | CTTT | 149 | GCCUGGAGAUGCAUACUCACA |
| crCTLA4_11 | CTTC | 150 | GGCAGGCUGACAGCCAGGUGA |
| crCTLA4_12 | CTTC | 151 | AGUCACCUGGCUGUCAGCCUG |
| crCTLA4_13 | CTTC | 152 | CUAGAUGAUUCCAUCUGCACG |
| crCTLA4_14 | CTTG | 153 | CCUUGGAUUUCAGCGGCACAA |
| crCTLA4_15 | CTTG | 154 | AUUUCCACUGGAGGUGCCCGU |
| crCTLA4_16 | CTTG | 155 | GAUAGUGAGGUUCACUUGAUU |
| crCTLA4_17 | CTTG | 156 | CAGAUGUAGAGUCCCGUGUCC |
| crCTLA4_18 | TTTG | 157 | CUCACCAAUUACAUAAAUCUG |
| crCTLA4_19 | CTTT | 158 | GCUCACCAAUUACAUAAAUCU |
| crCTLA4_20 | CTTT | 159 | GUUUUCUGUUGCAGAUCCAGA |
| crCTLA4_21 | TTTG | 160 | UUUUCUGUUGCAGAUCCAGAA |
| crCTLA4_22 | TTTT | 161 | CUGUUGCAGAUCCAGAACCGU |
| crCTLA4_23 | CTTC | 162 | CUCCUCUGGAUCCUUGCAGCA |
| crCTLA4_24 | CTTG | 163 | CAGCAGUUAGUUCGGGGUUGU |
| crCTLA4_25 | CTTG | 164 | GAUUUCAGCGGCACAAGGCUC |
| crCTLA4_26 | TTTT | 165 | UUUAUAGCUUUCUCCUCACAG |
| crCTLA4_27 | CTTT | 166 | CUCCUCACAGCUGUUUCUUUG |
| crCTLA4_28 | TTTC | 167 | UCCUCACAGCUGUUUCUUUGA |
| crCTLA4_29 | TTTT | 168 | GCUCAAAGAAACAGCUGUGAG |
| crCTLA4_30 | TTTC | 169 | UUUUUGUGUUUGACAGCUAAA |
| crCTLA4_31 | TTTT | 170 | UGUGUUUGACAGCUAAAGAAA |
| crCTLA4_32 | TTTG | 171 | ACAGCUAAAGAAAAGAAGCCC |
| crCTLA4_33 | TTTT | 172 | CACAUAGACCCCUGUUGUAAG |
| crCTLA4_34 | TTTT | 173 | CACAUUCUGGCUCUGUUGGGG |
| crCTLA4_35 | CTTT | 174 | UCACAUUCUGGCUCUGUUGGG |
| crCTLA4_36 | TTTC | 175 | AGCCUUAUUUUAUUCCCAUCA |
| crCTLA4_37 | TTTC | 176 | UCAAUUGAUGGGAAUAAAAUA |
| crCTLA4_38 | TTTT | 177 | UUCUUCUCUUCAUCCCUGUCU |
| crCTLA4_39 | CTTT | 178 | GCAGAAGACAGGGAUGAAGAG |
| crCTLA4_40 | CTTT | 179 | GGCUUUUCCAUGCUAGCAAUG |
| crCTLA4_41 | TTTG | 180 | GCUUUUCCAUGCUAGCAAUGC |
| crLAG3_1 | TTTG | 181 | GGGUGCAUACCUGUCUGGCUG |
| crLAG3_2 | TTTG | 182 | GGUCACCUGGAUCCCUGGGGA |
| crLAG3_3 | TTTC | 183 | UCAGGACCUUGGCUGGAGGCA |
| crLAG3_4 | TTTC | 184 | CCAGCCUUGGCAAUGCCAGCU |
| crLAG3_5 | TTTG | 185 | UGAGGUGACUCCAGUAUCUGG |
| crLAG3_6 | CTTG | 186 | CUGUUUCUGCAGCCGCUUUGG |
| crLAG3_7 | CTTG | 187 | CACAGUGACUGCCAGCCCCCC |
| crLAG3_8 | TTTT | 188 | GAACUGCUCCUUCAGCCGCCC |
| crLAG3_9 | CTTC | 189 | AGCCGCCCUGACCGCCCAGCC |
| crLAG3_10 | TTTC | 190 | CGCUAAGUGGUGAUGGGGGGA |
| crLAG3_11 | CTTT | 191 | CCGCUAAGUGGUGAUGGGGGG |
| crLAG3_12 | CTTA | 192 | GCGGAAAGCUUCCUCUUCCUG |
| crLAG3_13 | CTTG | 193 | GGGCAGGAAGAGGAAGCUUUC |
| crLAG3_14 | CTTC | 194 | CUCUUCCUGCCCCAAGUCAGC |
| crLAG3_15 | CTTC | 195 | AACGUCUCCAUCAUGUAUAAC |
| crLAG3_16 | TTTT | 196 | CUUUUCUCUUCAGGUCUGGAG |
| crLAG3_17 | TTTC | 197 | UGCAGCCGCUUUGGGUGGCUC |
| crLAG3_18 | TTTT | 198 | CUCUUCAGGUCUGGAGCCCCC |
| crLAG3_19 | CTTG | 199 | ACAGUGUACGCUGGAGCAGGU |
| crLAG3_20 | CTTG | 200 | GCAGUGAGGAAAGACCGGGUC |
| crLAG3_21 | TTTC | 201 | CUCACUGCCAAGUGGACUCCU |
| crLAG3_22 | CTTT | 202 | ACCCUUCGACUAGAGGAUGUG |
| crLAG3_23 | TTTA | 203 | CCCUUCGACUAGAGGAUGUGA |
| crLAG3_24 | CTTC | 204 | GACUAGAGGAUGUGAGCCAGG |
| crLAG3_25 | TTTC | 205 | CCACCUGAGGCUGACCUGUGA |
| crLAG3_26 | CTTT | 206 | CCCACCUGAGGCUGACCUGUG |
| crLAG3_27 | CTTC | 207 | UACUCUUUUCAGUGACUCCCA |
| crLAG3_28 | TTTT | 208 | ACCUGGAGCCACCCAAAGCGG |
| crLAG3_29 | TTTT | 209 | CAGUGACUCCCAAAUCCUUUG |
| crLAG3_30 | CTTC | 210 | CCCAGGGAUCCAGGUGACCCA |
| crLAG3_31 | CTTT | 211 | GGGUCACCUGGAUCCCUGGGG |
| crLAG3_32 | CTTT | 212 | GUGAGGUGACUCCAGUAUCUG |
| crLAG3_33 | CTTT | 213 | GUGUGGAGCUCUCUGGACACC |
| crLAG3_34 | TTTG | 214 | UGUGGAGCUCUCUGGACACCC |
| crLAG3_35 | CTTG | 215 | GCUGGAGGCACAGGAGGCCCA |
| crLAG3_36 | TTTT | 216 | GCUCACCUAGUGAAGCCUCUC |
| crLAG3_37 | CTTT | 217 | CCCAGCCUUGGCAAUGCCAGC |
| crLAG3_38 | CTTG | 218 | GCAAUGCCAGCUGUACCAGGG |
| crLAG3_39 | CTTC | 219 | UUGGAGCAGCAGUGUACUUCA |
| crLAG3_40 | CTTC | 220 | ACAGAGCUGUCUAGCCCAGGU |
| crLAG3_41 | CTTT | 221 | CUCCAUAGGUGCCCAACGCUC |
| crLAG3_42 | TTTC | 222 | UCCAUAGGUGCCCAACGCUCU |
| crLAG3_43 | TTTC | 223 | UCAUCCUUGGUGUCCUUUCUC |
| crLAG3_44 | CTTG | 224 | GUGUCCUUUCUCUGCUCCUUU |
| crLAG3_45 | CTTT | 225 | CUCUGCUCCUUUUGGUGACUG |
| crLAG3_46 | CTTC | 226 | UGCGAAGAGCAGGGGUCACUU |
| crLAG3_47 | CTTT | 227 | UGGUGACUGGAGCCUUUGGCU |
| crLAG3_48 | TTTT | 228 | GGUGACUGGAGCCUUUGGCUU |
| crLAG3_49 | CTTT | 229 | GGCUUUCACCUUUGGAGAAGA |
| crLAG3_50 | TTTG | 230 | GCUUUCACCUUUGGAGAAGAC |
| crLAG3_51 | CTTG | 231 | CUCUAAGGCAGAAAAUCGUCU |
| crLAG3_52 | TTTT | 232 | CUGCCUUAGAGCAAGGGAUUC |
| crLAG3_53 | CTTA | 233 | GAGCAAGGGAUUCACCCUCCG |
| crLAG3_54 | TTTC | 234 | CCGCCCAGUGGCCCGCCCGCU |
| crLAG3_55 | CTTC | 235 | UCGCUAUGGCUGCGCCCAGCC |
| crLAG3_56 | TTTA | 236 | UCCUUGCACAGUGACUGCCAG |
| crPDCD1_1 | TTTA | 237 | GCACGAAGCUCUCCGAUGUGU |
| crPDCD1_2 | TTTC | 238 | UCUGCAGGGACAAUAGGAGCC |
| crPDCD1_3 | TTTC | 239 | CAGUGGCGAGAGAAGACCCCG |
| crPDCD1_4 | TTTC | 240 | CUAGCGGAAUGGGCACCUCAU |
| crPDCD1_5 | CTTC | 241 | GUGCUAAACUGGUACCGCAUG |
| crPDCD1_6 | CTTC | 242 | AACCUGACCUGGGACAGUUUC |
| crPDCD1_7 | CTTG | 243 | UCCGUCUGGUUGCUGGGGCUC |
| crPDCD1_8 | CTTC | 244 | CCCGAGGACCGCAGCCAGCCC |
| crPDCD1_9 | CTTC | 245 | CGUGUCACACAACUGCCCAAC |
| crPDCD1_10 | CTTC | 246 | CACAUGAGCGUGGUCAGGGCC |
| crPDCD1_11 | CTTT | 247 | GAUCUGCGCCUUGGGGGCCAG |
| crPDCD1_12 | TTTG | 248 | AUCUGCGCCUUGGGGGCCAGG |
| crPDCD1_13 | CTTG | 249 | GGGGCCAGGGAGAUGGCCCCA |
| crPDCD1_14 | CTTT | 250 | GUGCCCUUCCAGAGAGAAGGG |
| crPDCD1_15 | TTTG | 251 | UGCCCUUCCAGAGAGAAGGGC |
| crPDCD1_16 | TTTC | 252 | CCUUCCGCUCACCUCCGCCUG |
| crPDCD1_17 | CTTC | 253 | CAGAGAGAAGGGCAGAAGUGC |
| crPDCD1_18 | CTTC | 254 | UGCCCUUCUCUCUGGAAGGGC |
| crPDCD1_19 | TTTG | 255 | GAACUGGCCGGCUGGCCUGGG |
| crPDCD1_20 | CTTT | 256 | CUCCUCAAAGAAGGAGGACCC |
| crPDCD1_21 | TTTC | 257 | UCCUCAAAGAAGGAGGACCCC |
| crPDCD1_22 | CTTC | 258 | UCUCGCCACUGGAAAUCCAGC |
| crPDCD1_23 | CTTT | 259 | CCUAGCGGAAUGGGCACCUCA |
| crPDCD1_24 | CTTC | 260 | CGCUCACCUCCGCCUGAGCAG |
| crPDCD1_25 | CTTG | 261 | GCCCCUCUGACCGGCUUCCUU |
| crPDCD1_26 | CTTC | 262 | UCCACUGCUCAGGCGGAGGUG |
| crPDCD1_27 | CTTC | 263 | UCCCCAGCCCUGCUCGUGGUG |
| crPDCD1_28 | CTTC | 264 | GGUCACCACGAGCAGGGCUGG |
| crPDCD1_29 | CTTC | 265 | ACCUGCAGCUUCUCCAACACA |
| crPDCD1_30 | CTTC | 266 | UCCAACACAUCGGAGAGCUUC |
| crPTPN1_1 | TTTA | 267 | CCUGACAGCGAAUCAUAACAU |
| crPTPN1_2 | TTTC | 268 | AUUCCAACUUACCUAACGGAA |
| crPTPN1_3 | TTTC | 269 | UGUGCGCACUGGUGAUGACAA |
| crPTPN11_4 | TTTC | 270 | CAAUCUGCUCACCUGCUUGAG |
| crPTPN11_5 | TTTC | 27 | UUCUAGUUGAUCAUACCAGGG |
| crPTPN11_6 | TTTA | 272 | AUAACUUACCUCAAAUUCUUC |
| crPTPN11_7 | CTTA | 273 | CCUAACGGAAAGUGUGAAGUC |
| crPTPN11_8 | TTTC | 274 | CAGACACUACAACAACAGGAG |
| crPTPN11_9 | TTTA | 275 | GGUGGUUUCAUGGACAUCUCU |
| crPTPN11_10 | TTTC | 276 | CCAGAGAGAUGUCCAUGAAAC |
| crPTPN6_1 | TTTC | 277 | UAUGACCUGUAUGGAGGGGAG |
| crPTPN6_2 | TTTG | 278 | CGACUCUGACAGAGCUGGUGG |
| crPTPN6_3 | TTTG | 279 | CAGAAGCAGGAGGUGAAGAAC |
| crPTPN6_4 | TTTG | 280 | ACUGCCCCCCACCCAGGCCUG |
| crPTPN6_5 | CTTA | 281 | UGGGCCCUACUCUGUGACCAA |
| crPTPN6_6 | TTTC | 282 | ACCGAGACCUCAGUGGGCUGG |
| crPTPN6_7 | CTTC | 283 | UCUAGGUGGUACCAUGGCCAC |
| crPTPN6_8 | CTTG | 284 | GCCUGCAGCAGCGUCUCUGCC |
| crPTPN6_9 | TTTC | 285 | UUGUGCGUGAGAGCCUCAGCC |
| crPTPN6_10 | CTTC | 286 | GUGCUUUCUGUGCUCAGUGAC |
| crPTPN6_11 | CTTG | 287 | GGCUGGUCACUGAGCACAGAA |
| crPTPN6_12 | CTTT | 288 | CUGUGCUCAGUGACCAGCCCA |
| crPTPN6_13 | TTTC | 289 | UGUGCUCAGUGACCAGCCCAA |
| crPTPN6_14 | CTTG | 290 | AUGUGGGUGACCCUGAGCGGG |
| crPTPN6_15 | CTTA | 291 | CCUCGCACAUGACCUUGAUGU |
| crPTPN6_16 | TTTG | 292 | GCUCCCCCCAGGGUGGACGCU |
| crPTPN6_17 | CTTG | 293 | AGCAGGGUCUCUGCAUCCAGC |
| crPTPN6_18 | TTTG | 294 | GAGACCUUCGACAGCCUCACG |
| crPTPN6_19 | CTTC | 295 | GACAGCCUCACGGACCUGGUG |
| crPTPN6_20 | TTTC | 296 | AAGAAGACGGGGAUUGAGGAG |
| crPTPN6_21 | CTTC | 297 | UUGUUCAGUUCCAACACUCGG |
| crPTPN6_22 | CTTG | 298 | GCUGUAUCCUCGGACUCCUGC |
| crPTPN6_23 | TTTC | 299 | CCCACCCACAUCUCAGAGUUU |
| crPTPN6_24 | CTTC | 300 | CAGACGCUGGUGCAAGUUCUU |
| crPTPN6_25 | CTTG | 301 | CACCAGCGUCUGGAAGGGCAG |
| crPTPN6_26 | CTTG | 302 | UUCUCUGGCCGCUGCCCUUCC |
| crPTPN6_27 | CTTG | 303 | AUGUAGUUGGCAUUGAUGUAG |
| crPTPN6_28 | CTTG | 304 | CGUCCAGAACCAGCUGCUAGG |
| crPTPN6_29 | CTTC | 305 | UGGCAGAUGGCGUGGCAGGAG |
| crPTPN6_30 | TTTC | 306 | UCCACCUCUCGGGUGGUCAUG |
| crPTPN6_31 | CTTT | 307 | CUCCACCUCUCGGGUGGUCAU |
| crPTPN6_32 | CTTT | 308 | CCAGAACAAAUGCGUCCCAUA |
| crPTPN6_33 | TTTC | 309 | CAGAACAAAUGCGUCCCAUAC |
| crPTPN6_34 | TTTG | 310 | UAUUCGGUUGUGUCAUGCUCC |
| crPTPN6_35 | CTTA | 311 | CAGGUCUCCCCGCUGGACAAU |
| crPTPN6_36 | CTTC | 312 | CUGGCUCGGCCCAGUCGCAAG |
| crPTPN6_37 | CTTA | 313 | GGGAGACCUGAUUCGGGAGAU |
| crPTPN6_38 | CTTC | 314 | CUGGACCAGAUCAACCAGCGG |
| crPTPN6_39 | TTTC | 315 | CUGCCGCUGGUUGAUCUGGUC |
| crPTPN6_40 | CTTT | 316 | CCUGCCGCUGGUUGAUCUGGU |
| crPTPN6_41 | CTTG | 317 | GUGGAGAUGUUCUCCAUGAGC |
| crPTPN6_42 | CTTG | 318 | UACUGCGCCUCCGUCUGCACC |
| crPTPN6_43 | TTTC | 319 | AAUGAACUGGGCGAUGGCCAC |
| crPTPN6_44 | CTTC | 320 | UUCUUAGUGGUUUCAAUGAAC |
| crPTPN6_45 | CTTC | 321 | UCCCCUCCAUACAGGUCAUAG |
| crPTPN6_46 | CTTG | 322 | GAGUCUAGUGCAGGGACCGUG |
| crPTPN6_47 | CTTG | 323 | CCCCCCUGCACCCGGCUGCAG |
| crPTPN6_48 | CTTG | 324 | UGUCUGCAGCCGGGUGCAGGG |
| crPTPN6_49 | TTTC | 325 | UCCUCCCUCUUGUUCUUAGUG |
| crPTPN6_50 | CTTT | 326 | CUCCUCCCUCUUGUUCUUAGU |
| crPTPN6_51 | CTTC | 327 | UUCACUUUCUCCUCCCUCUUG |
| crPTPN6_52 | CTTG | 328 | AGGUGGAUGAUGGUGCCGUCG |
| crPTPN6_53 | CTTC | 329 | CCUGACGCUGCCUUCUCUAGG |
| crTIGIT_1 | TTTC | 330 | AGGCCUUACCUGAGGCGAGGG |
| crTIGIT_2 | TTTT | 331 | GUCCUCCCUCUAGUGGCUGAG |
| crTIGIT_3 | CTTG | 332 | GGGUGGCACAUCUCCCCAUCC |
| crTIGIT_4 | TTTC | 333 | UGCAGAGAAAGGUGGCUCUAU |
| crTIGIT_5 | TTTG | 334 | UAAUGCUGACUUGGGGUGGCA |
| crTIGIT_6 | CTTA | 335 | CCUGAGGCGAGGGGAGCCUGC |
| crTIGIT_7 | CTTG | 336 | AAGGAUGGGGAGAUGUGCCAC |
| crTIGIT_8 | CTTC | 337 | AAGGAUCGAGUGGCCCCAGGU |
| crTIGIT_9 | CTTC | 338 | UGCAUCUAUCACACCUACCCU |
| crTIGIT_10 | TTTC | 339 | UAGGACCUCCAGGAAGAUUCU |
| crTIGIT_11 | CTTT | 340 | CUAGGACCUCCAGGAAGAUUC |
| crTIGIT_12 | CTTG | 341 | CUCCAGCAGGAAUACCUGAGC |
| crTIGIT_13 | CTTG | 342 | GAGCCAUGGCCGCGACGCUGG |
| crTIGIT_14 | TTTC | 343 | UAGUCAACGCGACCACCACGA |
| crTIGIT_15 | CTTT | 344 | CUAGUCAACGCGACCACCACG |
| crTIGIT_16 | TTTG | 345 | UAGUUUGUUUGUUUUUAGAAG |
| crTIGIT_17 | TTTG | 346 | UUUGUUUUUAGAAGAAAGCCC |
| crTIGIT_18 | TTTG | 347 | UUUUUAGAAGAAAGCCCUCAG |
| crTIGIT_19 | TTTT | 348 | UAGAAGAAAGCCCUCAGAAUC |
| crTIGIT_20 | CTTC | 349 | CACAGAAUGGAUUCUGAGGGC |
| crTIGIT_21 | TTTT | 350 | CUCCUGAGGUCACCUUCCACA |
| crTIGIT_22 | CTTC | 351 | CUGGGGGUGAGGGAGCACUGG |
| crTIGIT_23 | CTTC | 352 | UGCCUGGACACAGCUUCCUGG |
| crTIGIT_24 | CTTC | 353 | GUCCUCUUCCCUAGGAAUGAU |
| crTIGIT_25 | CTTC | 354 | UGUAACUCAGGACAUUGAAGU |
| crTIGIT_26 | CTTC | 355 | AAUGUCCUGAGUUACAGAAGC |
| crTIGIT_27 | TTTC | 356 | UAUUGUGCCUGUCAUCAUUCC |
| crTIGIT_28 | TTTC | 357 | UCUGCAGAAAUGUUCCCCGUU |
| crTIGIT_29 | CTTT | 358 | CUCUGCAGAAAUGUUCCCCGU |
| crTIGIT_30 | CTTG | 359 | UGCCGUGGUGGAGGAGAGGUG |
| crTIGIT_31 | CTTC | 360 | UGGCCAUUUGUAAUGCUGACU |
| crTIM3_1 | CTTA | 361 | CUUGUAAGUAGUAGCAGCAGC |
| crTIM3_2 | TTTC | 362 | CAAGGAUGCUUACCACCAGGG |
| crTIM3_3 | CTTG | 363 | UAAGUAGUAGCAGCAGCAGCA |
| crTIM3_4 | CTTA | 364 | CCACCAGGGGACAUGGCCCAG |
| crTIM3_5 | TTTG | 365 | AAUGUGGCAACGUGGUGCUCA |
| crTIM3_6 | CTTT | 366 | UCUUCUGCAAGCUCCAUGUUU |
| crTIM3_7 | CTTT | 367 | GCCCCAGCAGACGGGCACGAG |
| crTIM3_8 | TTTC | 368 | AUCAGUCCUGAGCACCACGUU |
| crTIM3_9 | CTTT | 369 | CAUCAGUCCUGAGCACCACGU |
| crTIM3_10 | TTTA | 370 | GCCAGUAUCUGGAUGUCCAAU |
| crTIM3_11 | TTTG | 371 | CGGAAAUCCCCAUUUAGCCAG |
| crTIM3_12 | CTTT | 372 | GCGGAAAUCCCCAUUUAGCCA |
| crTIM3_13 | TTTC | 373 | CGCAAAGGAGAUGUGUCCCUG |
| crTIM3_14 | TTTG | 374 | GAUCCGGCAGCAGUAGAUCCC |
| crTIM3_15 | TTTT | 375 | UCAUCAUUCAUUAUGCCUGGG |
| crTIM3_16 | TTTT | 376 | CUUCUGCAAGCUCCAUGUUUU |
| crTIM3_17 | CTTC | 377 | AGGUUAAAUUUUUCAUCAUUC |
| crTIM3_18 | TTTG | 378 | AUGACCAACUUCAGGUUAAAU |
| crTIM3_19 | TTTA | 379 | ACCUGAAGUUGGUCAUCAAAC |
| crTIM3_20 | CTTA | 380 | UGUUGUUUCUGACAUUAGCCA |
| crTIM3_21 | TTTC | 381 | UGACAUUAGCCAAGGUCACCC |
| crTIM3_22 | CTTG | 382 | GAAAGGCUGCAGUGAAGUCUC |
| crTIM3_23 | CTTC | 383 | ACUGCAGCCUUUCCAAGGAUG |
| crTIM3_24 | CTTT | 384 | CCAAGGAUGCUUACCACCAGG |
| crTIM3_25 | TTTT | 385 | CACAUCUUCCCUUUGACUGUG |
| crTIM3_26 | TTTT | 386 | UAUAGCAGAGACACAGACACU |
| crTIM3_27 | TTTA | 387 | UAUCAGGGAGGCUCCCCAGUG |
| crTIM3_28 | CTTA | 388 | CUGUUAGAUUUAUAUCAGGGA |
| crTIM3_29 | TTTG | 389 | UGUUUCCAUAGCAAAUAUCCA |
| crTIM3_30 | TTTC | 390 | CAUAGCAAAUAUCCACAUUGG |
| crTIM3_31 | CTTA | 391 | CGGGACUCUGGAGCAACCAUC |
| crTIM3_32 | TTTG | 392 | AAAAUUAAAGCGCCGAAGAUA |
| crTIM3_33 | CTTA | 393 | CAUUUGAAAAUUAAAGCGCCG |
| crTIM3_34 | CTTT | 394 | UGUUUCCCCCUUACUAGGGUA |
| crTIM3_35 | TTTT | 395 | GUUUCCCCCUUACUAGGGUAU |
| crTIM3_36 | CTTT | 396 | GACUGUGUCCUGCUGCUGCUG |
| crTIM3_37 | TTTC | 397 | CCCCUUACUAGGGUAUUCUCA |
| crTIM3_38 | CTTA | 398 | CUAGGGUAUUCUCAUAGCAAA |
| crTIM3_39 | CTTA | 399 | AAUUCUGUAUCUUCUCUUUGC |
| crTIM3_40 | CTTT | 400 | AUUUCCACAGCCUCAUCUCUU |
| crTIM3_41 | TTTA | 401 | UUUCCACAGCCUCAUCUCUUU |
| crTIM3_42 | TTTC | 402 | CACAGCCUCAUCUCUUUGGCC |
| crTIM3_43 | TTTG | 403 | GCCAACCUCCCUCCCUCAGGA |
| crTIM3_44 | TTTG | 404 | CCAAUCCUGAGGGAGGGAGGU |
| crTIM3_45 | TTTT | 405 | CUUCUGAGCGAAUUCCCUCUG |
| crTIM3_46 | CTTC | 406 | AUAUACGUUCUCUUCAAUGGU |
| crTIM3_47 | CTTT | 407 | GGGUUGUCGCUUUGCAAUGCC |
| crTIM3_48 | TTTG | 408 | GGUUGUCGCUUUGCAAUGCCA |
| crTIM3_49 | CTTC | 409 | UCUCUCUAUGCAGGGUCCUCA |
| crTIM3_50 | CTTC | 410 | UACACCCCAGCCGCCCCAGGG |
| crTIM3_51 | TTTG | 411 | CCCCAGCAGACGGGCACGAGG |
| crAAVS1 | TTTC | 412 | TTAGGATGGCCTTCTCCGACG |

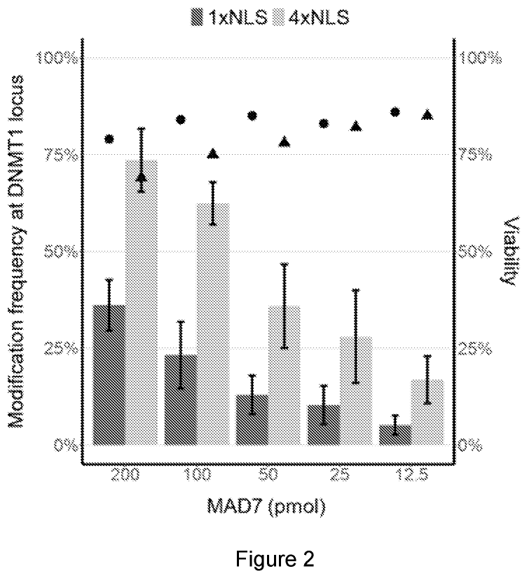

Firstly, using a gNA targeting the DNMT1 locus, the editing frequency of MAD7 comprising either one or four NLS complexed with the respective gNA was compared. RNP concentration-dependent modification efficiency was observed as evidenced by an increased fraction of modified amplicons (FIG. 2, left axis, dark grey for MAD7-1NLS and light grey representing MAD7-4NLS). Error bars represent one standard deviation for a sample of 3 (n=3). In this experiment, editing frequency was enhanced in Jurkat cells when treated with RNPs comprising MAD-4NLS, which indicates that optimization of the NLS can improve editing efficiency. A slight decrease in cell viability was seen at higher concentrations of RNP for those comprising four NLS as compared to one NLS (FIG. 2, right axis). Specifically, FIG. 2 shows editing frequency at the DNMT1 locus (n=3; Mean±SD) and cell viability of T-cell leukemic cells as a function of MAD7 comprising one or four nuclear localization signal (NLS) and MAD7-RNP amounts (pmol; constant ratio of 1:1.5 MAD7:gNA). Dark grey bars and circles represent mean modification frequency and viability using MAD7-INLS, respectively. Light grey bars and triangles represent mean modification frequency and viability using MAD7-4NLS, respectively.

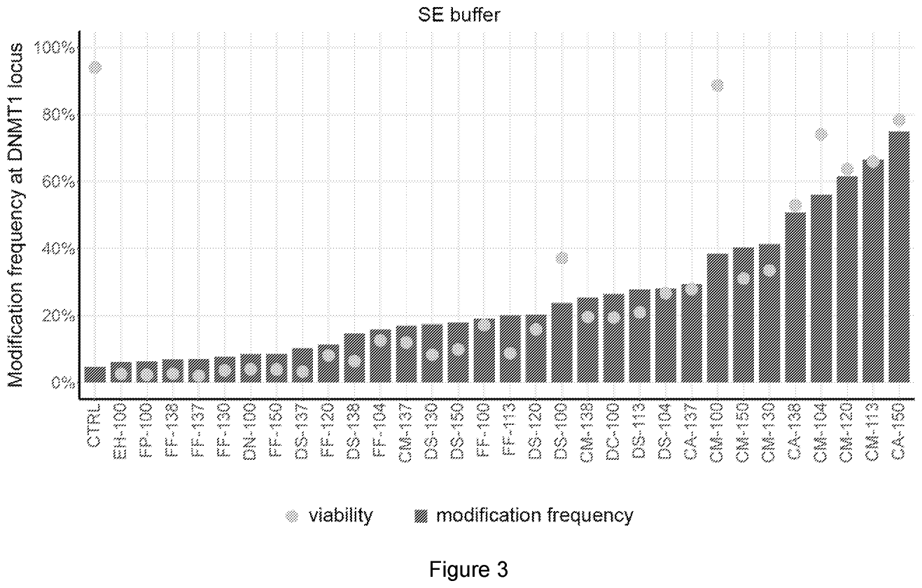

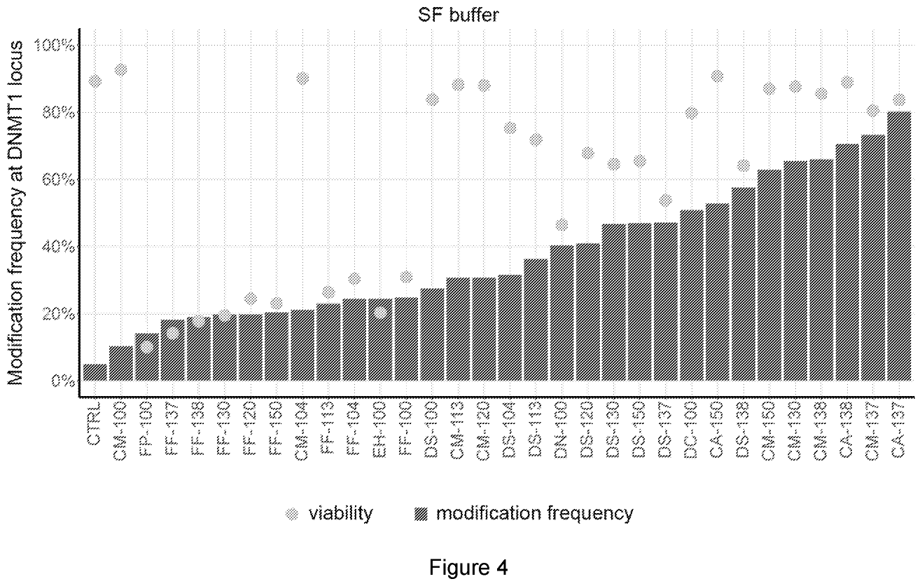

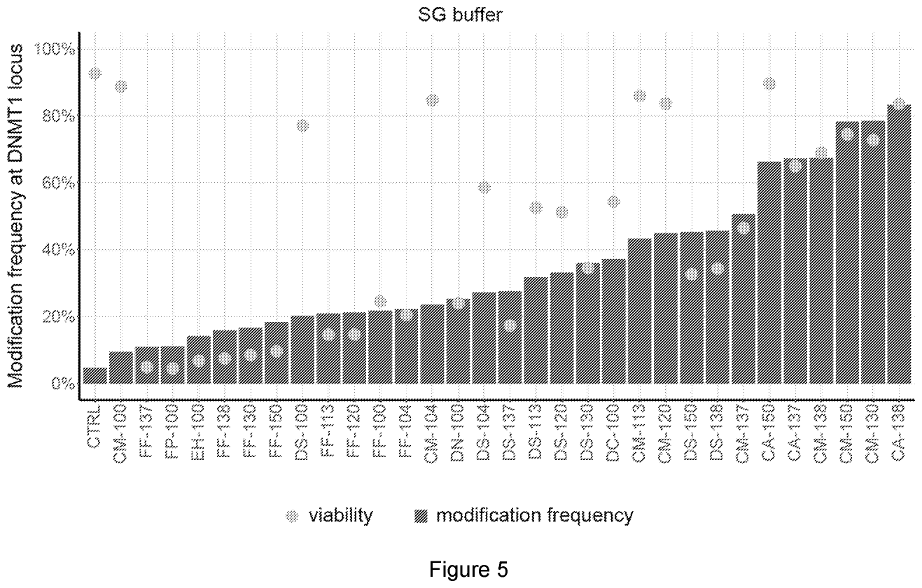

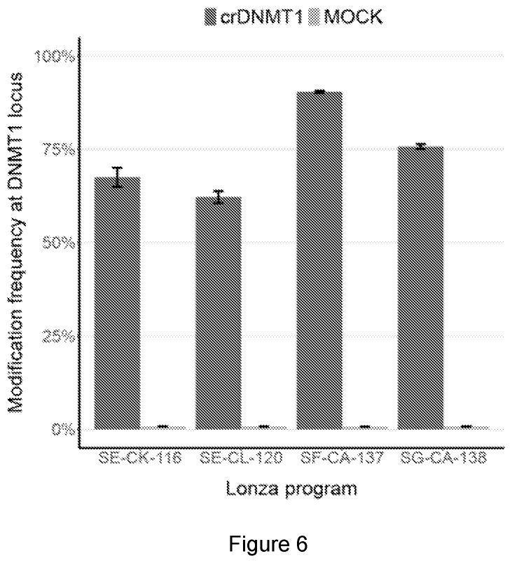

To optimize editing activity, 93 different transfection conditions were tested; 31 nucleofection programs in combination with three buffers-on the Lonza Nucleofector 96-well Shuttle System (FIGS. 3-5). FIGS. 3, 4, and 5 show the editing frequency (bars; x-axis) of each of the electroporation conditions (buffers SE, SF, and SG respectively) as compared to a control (y-axis, control at the top). The majority of buffer-program transfection combinations resulted in suboptimal viability (dots; x-axis) and editing frequency, however, the analysis revealed several conditions that supported substantial rates of both cell viability and editing. Two improved conditions observed in the screen, namely SF-CA-137 and SG-CA-138, were then validated and compared to the Lonza recommended nucleofection programs for T-cell leukemia, namely SE-CL-120 and SE-CK-116 (FIG. 6). Specifically, FIG. 6 shows editing frequency at the DNMT1 locus (n=4; Mean±SD) in T-cell leukemic cell line achieved by utilization of the transfection conditions identified in FIG. 2 (100 pmol MAD7-4NLS) and Lonza recommended nucleofection programs SE-CK-116 and SE-CL-120, as well as the two best nucleofection programs observed in this study, SF-CA-137 and SG-CA-138 (FIGS. 3-5). Dark grey bars represent mean modification frequency using crDNMT1. Light grey bars represent mean modification frequency using crIDTneg (Integrated DNA Technologies, IDT).

### Example 12: Scalable High-Level MAD7-RNP Editing of Immunologically Relevant Genes in Jurkate T-Cell Leukemia Cell Line

The Jurkat T-cell leukemia cell line was used as a model system to screen GNAs demonstrating high editing efficiency. The screen included 298 unique gNAs comprising one or more spacer sequences of SEQ ID NOs: 86-384 of Table 1 targeting the immune checkpoint receptors PDCD1, TIM3, LAG3, TIGIT, and CTLA4, the checkpoint phosphatases PTPN6 (SHP-1) and PTPN11 (SHP-2), and the TCR signaling subunit CD247 (CD3ζ). RNPs were generated as described in Example 3, nucleofected (Example 5), genomic DNA was extracted (Example 8), the edited loci amplified and sequenced (Example 9), and the sequencing data computationally analyzed (Example 10) using the CRISPResso2 algorithm.

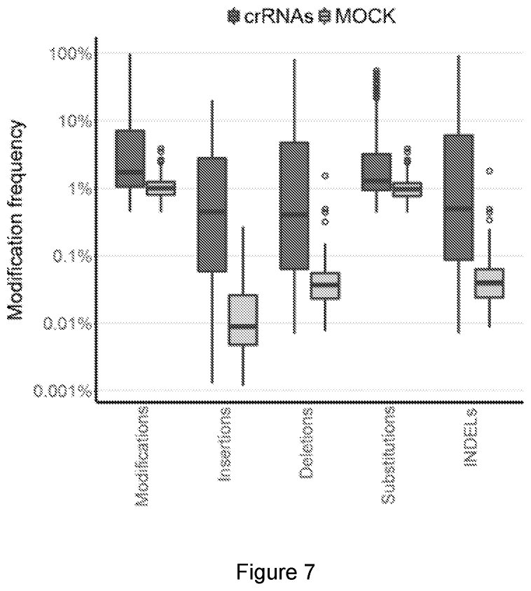

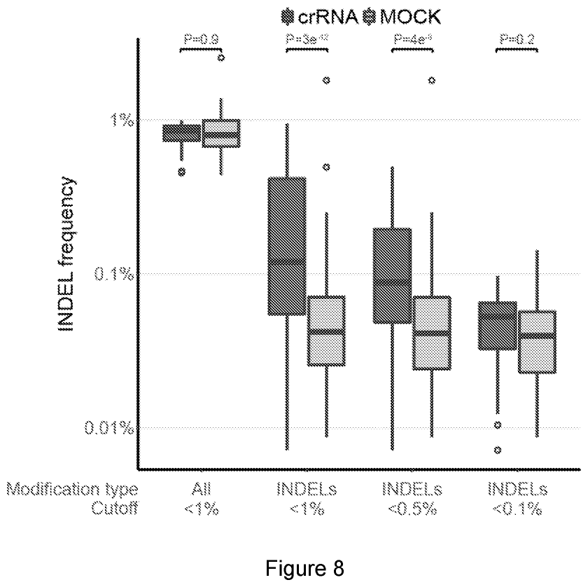

CRISPResso2 software reports the frequency of modifications (insertions, deletions, and substitutions) within a quantification window flanking the position of MAD7-induced cleavage in the amplicon sequence. To better understand detection of editing events, the type of modifications detected in 230 amplicons that were sequenced in both gNA-treated and MOCK samples (no MAD7) were compared. Relatively high modification frequencies (median 1%) in MOCK reactions were observed as a result of high frequency of substitutions (FIG. 7, light grey bars); substitutions were detected at a median frequency of 0.96%, likely due to the errors in NGS base calling or substitutions arising during DNA amplification, while insertions and deletions were found at a much lower median frequency of 0.003% and 0.042%, respectively. Specifically, FIG. 7 shows editing frequency at eight different loci using 298 gNAs (n=3; Mean±SD) in T-cell leukemic cell line as a function of various editing types: all modifications, only insertions, only deletions, only substitutions, or insertions and deletions (INDELs). Edits were achieved using the transfection conditions identified in Example 11, FIG. 2 (100 pmol MAD7-4NLS) and one of the tested Lonza nucleofection programs (FIG. 6; SF-CA-137). Dark grey boxplots represent mean modification frequency using gNAs. Light grey boxplots represent mean modification frequency using crIDTneg (IDT). Thus, the frequency of both insertions and deletions (INDEL) were used as a means to quantify the editing activity of the CRISPR-MAD7 system to minimize low end noise. Moreover, low INDEL frequencies in MOCK reactions enabled sensitive detection of editing events at a significantly greater fraction of sites (Fisher exact test, P=3×10⁻¹²; FIG. 8). Analysis of gNAs with low INDEL frequencies showed statistically significant editing in gNA-treated samples compared to MOCK samples at

INDEL frequencies as low as 0.5% (Fisher exact test, P=4×10⁻⁸; FIG. 8). This indicates the sensitivity of the assay to detect modifications in the sub-1% range. Specifically, FIG. 8 shows INDEL frequency at eight different loci using 298 gNAs (n=3; Mean±SD) in T-cell leukemic cell line as a function of two modification types: all modifications <1%, and INDELs <1%, or <0.5%, or <0.1%, with lower INDEL frequencies in MOCK compared to gNA reactions at INDELs <1% (Fisher's exact test; P=3×10⁻¹²) and <0.5% (Fisher exact test, P=4×10⁻⁸). Dark grey boxplots represent mean INDEL frequency using gNAs. Light grey boxplots represent mean INDEL frequency using crIDTneg (IDT).

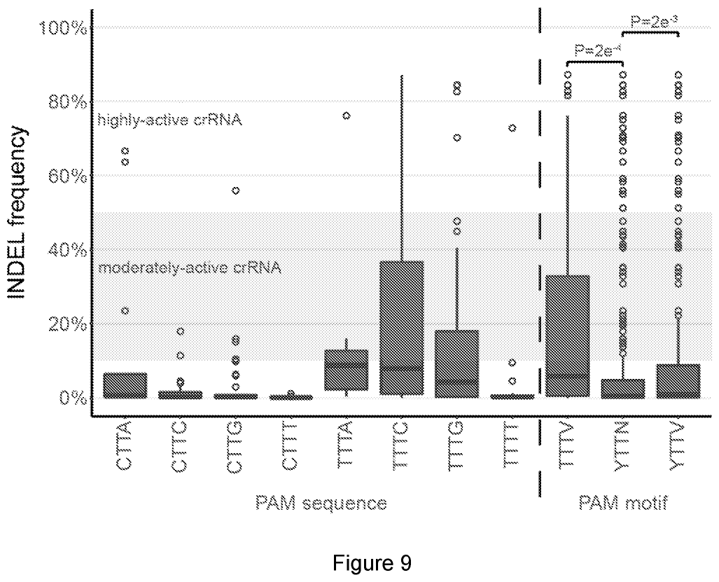

Since MAD7 can target a wide range of PAM, gNAs adjacent to all YTTN PAM variants were screened and editing specificity of MAD7 in Jurkat cells was analyzed. MAD7 demonstrated editing with all eight combinations of YTTN PAM; in this experiment, editing was higher at the YTTV and TTTV consensus sequences (Fisher exact test; P=2×10⁻³ and P=2×10⁻⁴ respectively). While the majority of highly-active (>50% INDEL frequency) gNAs were found at sites with YTTV and TTTV PAMs, moderately-active (>10% INDEL frequency) gNAs were found to target every PAM sequence with the exception of CTTT. This indicates that MAD7 can edit a wide range of target PAMs, albeit at reduced frequencies (FIG. 9). Specifically, FIG. 9 shows INDEL frequency at eight different loci using 298 gNAs (n=3; Mean±SD) in T-cell leukemic cell line as a function of eight YTTN PAM combinations, and TTTV, YTTN, and YTTV PAM motifs. A grey zone on the plot represents moderately-active gNAs (10-50% INDELs), the zone above highly-active gNAs (>50% INDELs), and the zone below active gNAs (1-10% INDELs). INDEL frequency at the YTTV and TTTV PAM motif is significantly higher compared to YTTN motif (Fisher exact test, P=2×10⁻³ and P=2×10⁻⁴, respectively).

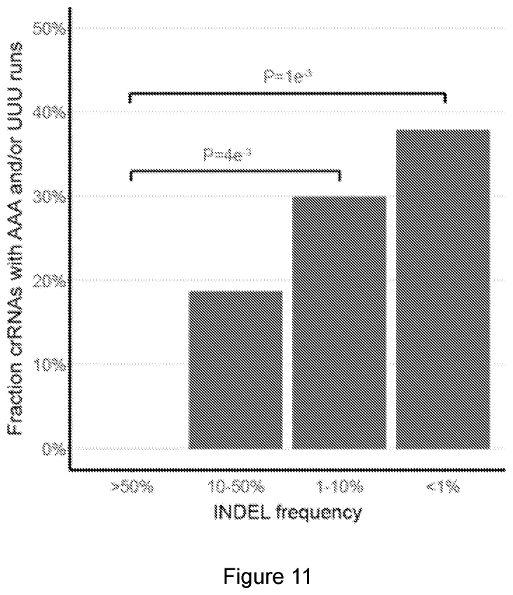

Given the large number of gNAs analyzed, it was determined if the targeted DNA sequence biases editing efficiency. Sequence logos were made to compare the DNA-complementary gNA sequences of inactive (<1% INDELs), active (1-10% INDELs), moderately-active (10-50% INDELs), and highly-active (>50% INDELs) gNAs (FIG. 10A). While no strong biases for ribonucleotides at specific positions were identified in this experiment, guanine appeared overrepresented and uracil underrepresented on moderately-active and highly-active gNAs. Next, the frequency of ribonucleotide bases were analyzed within the same four classes of gNAs (FIG. 10B). The analysis confirmed significant enrichment of guanine and depletion of uracil on highly-active gNAs. Specifically, FIG. 10 shows (A) sequence logos comparing DNA-complementary gNA sequences of highly-active (>50% INDELs), moderately-active (10-50% INDELs), active (1-10% INDELs), and inactive (<1% INDELs) gNAs show no strong biases for ribonucleotides at specific positions, however, guanine appeared overrepresented and uracil underrepresented on highly-active and moderately-active gNAs; (B) nucleotide frequency on inactive (<1% INDELs; dark grey box), active (1-10% INDELs; medium grey box), moderately-active (10-50% INDELs; light grey box), and highly-active (>50% INDELs; white box) gNAs, with significant enrichment of guanine and depletion of uracil on highly-active gNAs compared to inactive gNAs (Fisher exact test, P=4×10⁻³ and P=3×10⁻⁴, respectively). Also, significant enrichment of guanine-cytosine content and depletion of adenine-uracil content was observed on moderately-active gNAs compared to inactive gNAs (Fisher exact test, P=1×10⁻²). Moreover, the data showed that nearly 40% of inactive gNAs had runs of three or more adenine or uracil ribonucleotides, while none of the highly-active and <20% of moderately-active gNAs contained such runs (FIG. 11). These sequence features can act as an algorithm for selecting putative high-activity gNAs during initial rounds of screening, and could reduce the overall cost of identifying gNAs for various genes of interest. Specifically, FIG. 11 shows fraction of gNAs with AAA and/or UUU runs as a function of INDEL frequency of highly-active (>50% INDELs), moderately-active (10-50% INDELs), active (1-10% INDELs), and inactive (<1% INDELs) gNAs. Fraction of inactive (<1% INDELs) and active (1-10% INDELs) gNAs containing such runs is higher compared to highly-active (>50% INDELs) gNAs (Fisher exact test, P=1×10⁻³ and P=4×10⁻⁴, respectively).

### Example 13: Validation of gNAs for Gene Editing and Disruption of Immunologically Relevant Genes Using T-Cell Leukemia Line

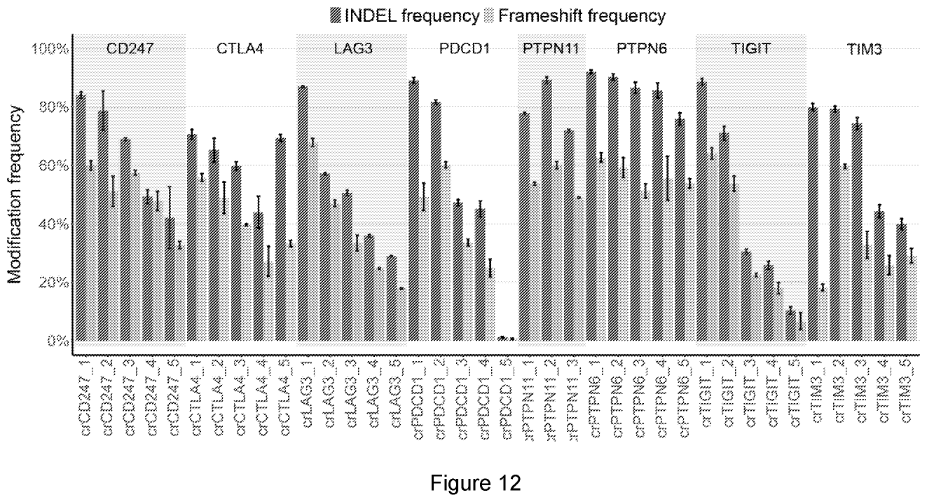

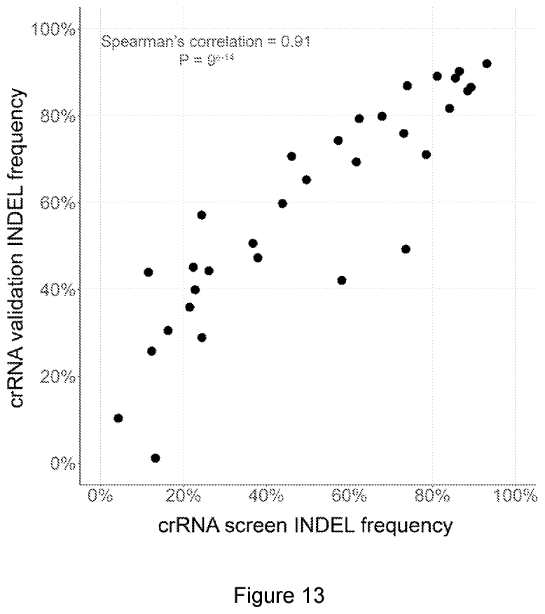

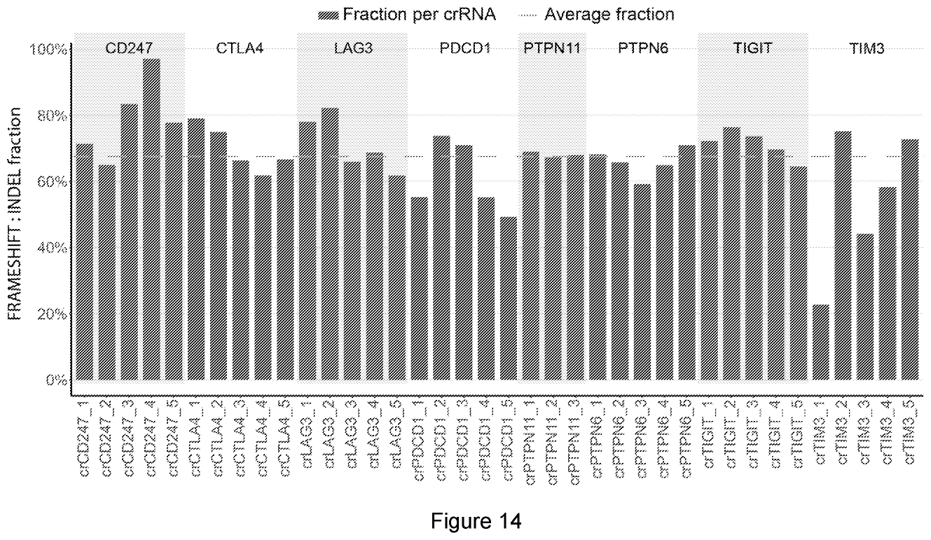

High-efficiency gNAs identified in our initial analysis were validated by assaying INDEL frequency for the top three or five gNAs for each of the selected immunologically relevant genes (FIG. 12). Specifically, FIG. 12 shows INDEL (dark grey bars) and frameshift (light grey bars) frequencies (n=3; Mean±SD) in T-cell leukemic cell line as a function of 38 high-efficiency gNAs. Alternating grey and white zones on the plot represent groups of three to five high-efficiency gNAs per locus. In the validation experiment, the INDEL frequency was significantly correlated to the measurements from the initial screen, highlighting the reproducibility of the INDEL assay (FIG. 13). Specifically, FIG. 13 shows correlation of INDEL frequency in the gNA validation experiment versus INDEL formation in the gNA screen experiment (Spearman's correlation=0.91; P=9×10⁻¹⁴), highlighting reproducibility of the INDEL assay. Using the CRISPresso2 software, the degree of open reading frame (ORF) disruption for each of the validated gNAs was estimated (FIG. 12). In addition, for four high-efficiency gNAs targeting three different exons at the PDCDI locus, surface expression of the PDCD1 protein was measured by flow cytometry 4, 7, and 11 days post-transfection (data not shown). The data revealed that the protein surface expression after transfection with crPDCD1_2, a gNA targeting the PDCD1 gene at the extracellular domain of the protein, was as low as 10% 4 days post-transfection and remained at this level even at day 11 post-transfection. The surface expression after transfection with the remaining three gNAs was significantly higher, 35% and 85% after transfection with crPDCD1_3 and both crPDCD1_4 and crPDCD1_5, respectively. This is in line with the ORF data analysis, which showed that for most of the gNAs including the high-efficiency crPDCDIs, the predicted number of INDELs leading to frameshifts was similar to that expected from an unbiased DNA repair process, with frameshifts in two-thirds of the edited loci (FIG. 14). However, several of the gNAs had a markedly different degree of ORF disruption; crCD247_4 resulted in frameshifts with 97% frequency, while crTIM3_1 and crTIM3_3 resulted in frameshifts with 23% and 44% frequency, respectively (FIG. 14). Specifically, FIG. 14 shows fraction of frameshift to INDEL frequency (dark grey bars) in T-cell leukemic cell line as a function of 38 high-efficiency gNAs. Average fraction of INDELs leading to frameshifts (dashed line) is approx. 66%. Alternating grey and white zones on the plot represent groups of three to five high-efficiency gNAs per locus. The analysis of repair products indicates that in the case of crTIM3_1, and to some extent crTIM3_3, the bias arose from directly repeated sequences at the DNA cleavage site, which possibly promoted microhomology-mediated end joining (MMEJ) repair following DNA cleavage. These data help inform selection of gNAs for gene KO since some gNAs, such as crTIM3_1, have much lower frequency of gene disruption than would be predicted based on the frequency of INDEL formation.

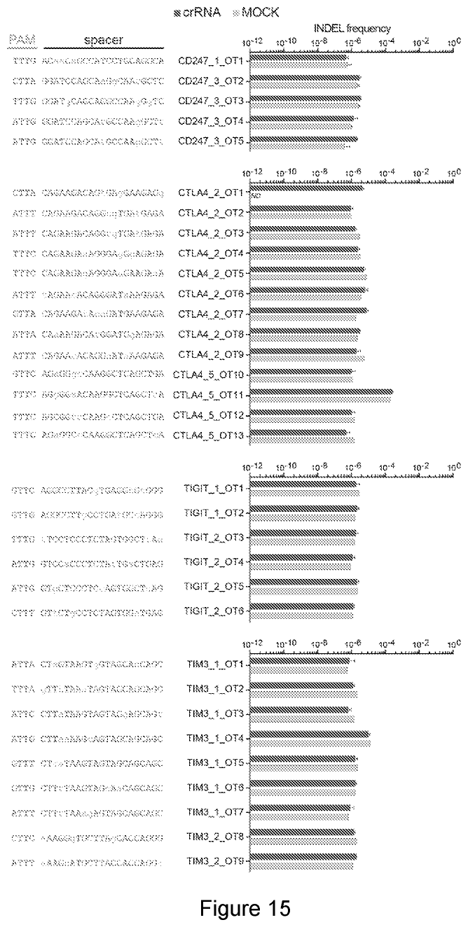

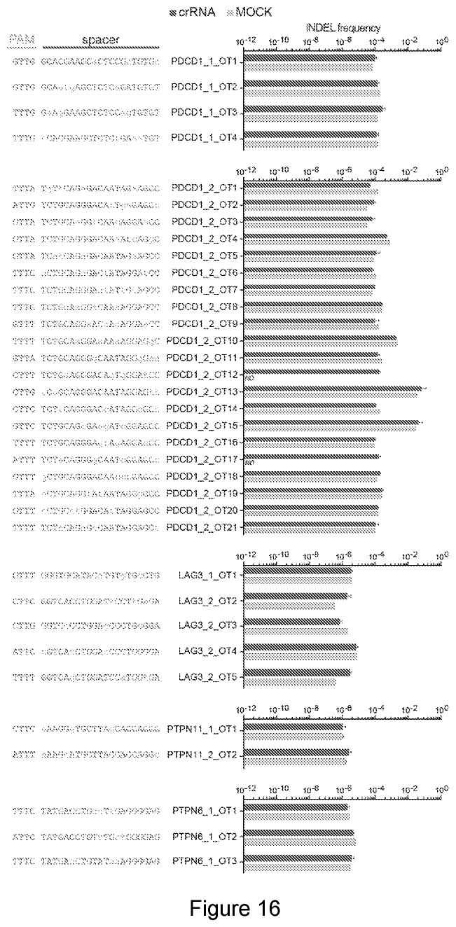

Another consideration for selecting gNAs is the potential for off-target cleavage events. The list of validated gNAs was analyzed using the CasOFFinder software to predict potential off-target editing sites in the genome with up to four mismatches between the gNA and the target DNA sequence. Using the Bioconductor R packages, the predicted off-target sites were matched with the human gene database, and those sites that targeted exons and introns within the genes were extracted. Afterwards, the degree of editing activity at these sites was examined by targeted next-generation sequencing, more specifically, at 25 predicted off-target sites for the top-two PDCD1 gNAs, i.e., crPDCD1_1 and crPDCD1_2. The analysis revealed low-level off-target activity at crPDCD1_2_13 and crPDCD1_2_15 sites, however, INDEL formation at these two sites was statistically insignificant compared to MOCK samples (non-targeting gNAs) (Pairwise T-test, P≥0.05; FIGS. 15 and 16). INDEL frequency at 43 putative off-target sites with up to three mismatches between gNA and target DNA sequence were assayed for the top-two gNAs targeting seven remaining genes (i.e., TIM3, LAG3, TIGIT, CTLA4, PTPN6, PTPN11, and CD247; spacer sequences in Table 1). The analysis revealed no detectable activity at any of the putative off-target sites (FIGS. 15 and 16), which confirms the high cleavage fidelity of MAD7-gNA complexes. Specifically, FIGS. 15-16 show INDEL frequency of MAD7 (n=3; Mean±SD) in T-cell leukemic cell line at predicted off-target sites analyzed by targeted deep sequencing. For crPDCD1, INDEL frequency was analyzed at the putative off-target editing sites with ≤4 mismatches between the gNA and target DNA sequence, and with≤3 mismatches on the remaining gNAs. PAM sequences and spacer sequences with mismatches marked in red are displayed next to their respective measured INDEL frequencies. No significant INDEL frequency at any of the off-target sites was detected (Pairwise T-test, P≥0.05).

Insertion of exogenous transgenes is an important aspect of mammalian cell engineering. Gene insertion with CRISPR-Cas is achieved by homology-directed repair of CRISPR-induced DNA breaks using HDR-donor templates to copy exogenous genetic sequences into targeted DNA loci. Several studies indicate that HDR templates, composed of linear double stranded DNA, provide the most robust and efficient method of transgene insertion using CRISPR-Cas genome editing systems.

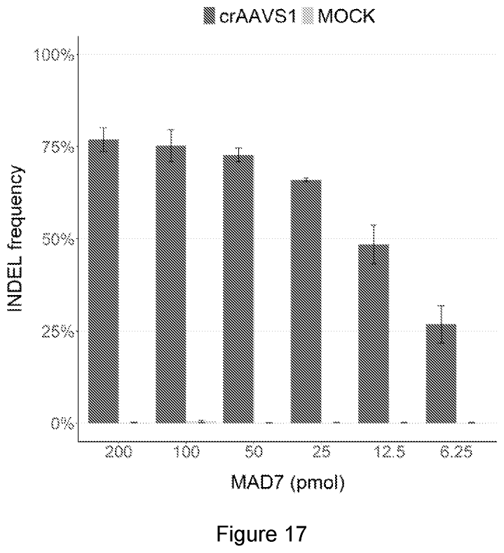

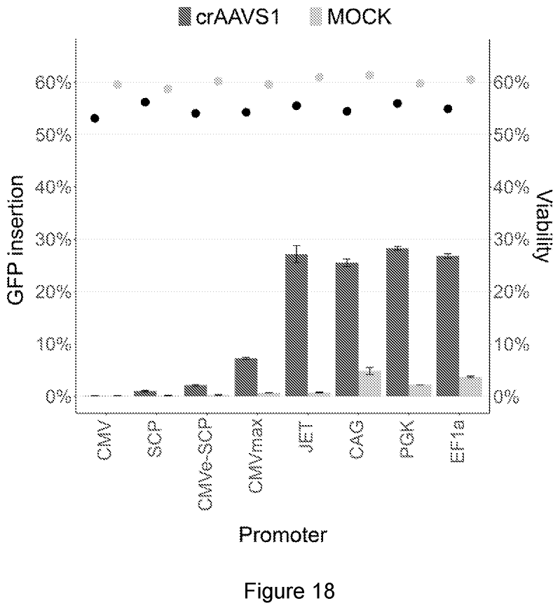

The Jurkat T-cell leukemia cell line was used to evaluate the transgene insertion and expression efficiency using CRISPR-MAD7 RNP complexes. A highly active gNA targeting the AAVS1 (spacer sequence in Table 1) safe-harbor locus (FIG. 17) was used in combination with eight different HDR-repair templates flanked with symmetric homology arms (HA) of 500 base pairs (bp) in the amount of 0.5 μg μL⁻¹. Specifically, FIG. 17 shows INDEL frequency at the AAVS1 locus (n=3; Mean±SD) in T-cell leukemic cell line as a function of MAD7-RNP amounts (pmol; constant ratio of 1:1.5 MAD7:gNA). Dark grey bars represent mean INDEL frequency using crAAVS1. Light grey bars represent mean modification frequency using crIDTneg (IDT). The HDR inserts comprised eight promoters (Table 2) differing in both size and promoter strength to drive GFP expression (FIG. 18). When the transient GFP expression diminished at day 14 post-transfection, comparable insertion efficiencies were observed with stable GFP expressions of up to 30% using four (JET, PGK, EF1a, and CAG) out of eight promoters (FIG. 18), suggesting that the insert size has not affected the integration efficiency at AAVS1 in human T-cell leukemia cell line. Specifically, FIG. 18 shows GFP insertion efficiency at AAVS1 (n=3; Mean±SD) and cell viability of T-cell leukemic cell line measured at day 14 post-transfection. HDR templates consisting of eight different promoters and flanked with symmetric homology arms of 500 base pairs in the amount of 0.5 μg μL⁻¹ were used. Size of promoters in base pairs: CMV, 1400; SCP, 970; CMVe-SCP, 1270; CMVmax, 1830; JET, 1100; CAG, 2600; PGK, 1410; EF-1a, 2090. Dark grey bars and circles present mean insertion frequency and cell viability using crAAVS1. Light grey bars represent mean insertion frequency and cell viability using crIDTneg (IDT).

TABLE 2

| | SEQ | |
|---|---|---|
| Name | ID NO | Sequence |
| CMV | 413 | CGTTACATAACTTACGGTAAATGGCCCGCCTGGCTGACCGCC |
| | | CAACGACCCCCGCCCATTGACGTCAATAATGACGTATGTTCC |
| | | CATAGTAACGCCAATAGGGACTTTCCATTGACGTCAATGGGT |
| | | GGAGTATTTACGGTAAACTGCCCACTTGGCAGTACATCAAGT |
| | | GTATCATATGCCAAGTACGCCCCCTATTGACGTCAATGACGG |
| | | TAAATGGCCCGCCTGGCATTATGCCCAGTACATGACCTTATG |
| | | GGACTTTCCTACTTGGCAGTACATCTACGTATTAGTCATCGC |
| | | TATTACCATGGTGATGCGGTTTTGGCAGTACATCAATGGGCG |
| | | TGGATAGCGGTTTGACTCACGGGGATTTCCAAGTCTCCACCC |
| | | CATTGACGTCAATGGGAGTTTGTTTTGGCACCAAAATCAACG |
| | | GGACTTTCCAAAATGTCGTAACAACTCCGCCCCATTGACGCA |
| | | AATGGGCGGTAGGCGTGTACGGTGGGAGGTCTATATAAGCA |
| | | GAGCT |
| SCP | 414 | GTACTTATATAAGGGGGTGGGGGCGCGTTCGTCCTCAGTCGC |
| | | GATCGAACACTCGAGCCGAGCAGACGTGCCTACGGACCG |
| CMVe- | 415 | CGTTACATAACTTACGGTAAATGGCCCGCCTGGCTGACCGCC |
| SCP | | CAACGACCCCCGCCCATTGACGTCAATAATGACGTATGTTCC |
| | | CATAGTAACGCCAATAGGGACTTTCCATTGACGTCAATGGGT |
| | | GGAGTATTTACGGTAAACTGCCCACTTGGCAGTACATCAAGT |
| | | GTATCATATGCCAAGTACGCCCCCTATTGACGTCAATGACGG |
| | | TAAATGGCCCGCCTGGCATTATGCCCAGTACATGACCTTATG |
| | | GGACTTTCCTACTTGGCAGTACATCTACGTATTAGTCATCGC |
| | | TATTACCATGGTACTTATATAAGGGGGTGGGGGCGCGTTCGT |
| | | CCTCAGTCGCGATCGAACACTCGAGCCGAGCAGACGTGCCT |
| | | ACGGACCG |
| CMVmax | 416 | TCAATATTGGCCATTAGCCATATTATTCATTGGTTATATAGC |
| | | ATAAATCAATATTGGCTATTGGCCATTGCATACGTTGTATCT |
| | | ATATCATAATATGTACATTTATATTGGCTCATGTCCAATATG |
| | | ACCGCCATGTTGGCATTGATTATTGACTAGTTATTAATAGTA |
| | | ATCAATTACGGGGTCATTAGTTCATAGCCCATATATGGAGTT |
| | | CCGCGTTACATAACTTACGGTAAATGGCCCGCCTGGCTGACC |
| | | GCCCAACGACCCCCGCCCATTGACGTCAATAATGACGTATGT |
| | | TCCCATAGTAACGCCAATAGGGACTTTCCATTGACGTCAATG |
| | | GGTGGAGTATTTACGGTAAACTGCCCACTTGGCAGTACATCA |
| | | AGTGTATCATATGCCAAGTCCGCCCCCTATTGACGTCAATGA |
| | | CGGTAAATGGCCCGCCTGGCATTATGCCCAGTACATGACCTT |
| | | ACGGGACTTTCCTACTTGGCAGTACATCTACGTATTAGTCAT |
| | | CGCTATTACCATGGTGATGCGGTTTTGGCAGTACACCAATGG |
| | | GCGTGGATAGCGGTTTGACTCACGGGGATTTCCAAGTCTCCA |
| | | CCCCATTGACGTCAATGGGAGTTTGTTTTGGCACCAAAATCA |
| | | ACGGGACTTTCCAAAATGTCGTAATAACCCCGCCCCGTTGAC |
| | | GCAAATGGGCGGTAGGCGTGTACGGTGGGAGGTCTATATAA |
| | | GCAGAGGTCGTTTAGTGAACCGTCAGATCACTAGTAGCTTTA |
| | | TTGCGGTAGTTTATCACAGTTAAATTGCTAACGCAGTCAGTG |
| | | CTCGACTGATCACAGGTAAGTATCAAGGTTACAAGACAGGT |
| | | TTAAGGAGGCCAATAGAAACTGGGCTTGTCGAGACAGAGAA |
| | | GATTCTTGCGTTTCTGATAGGCACCTATTGGTCTTACTGACAT |
| | | CCACTTTGCCTTTCTCTCCACAGGG |
| JET | 417 | GAATTCGGGCGGAGTTAGGGCGGAGCCAATCAGCGTGCGCC |
| | | GTTCCGAAAGTTGCCTTTTATGGCTGGGCGGAGAATGGGCGG |
| | | TGAACGCCGATGATTATATAAGGACGCGCCGGGTGTGGCAC |
| | | AGCTAGTTCCGTCGCAGCCGGGATTTGGGTCGCGGTTCTTGT |
| | | TTGTGGATCCCTGTGATCGTCACTTGACA |
| CAG | 418 | ATCTCGACTAGTTATTAATAGTAATCAATTACGGGGTCATTA |
| | | GTTCATAGCCCATATATGGAGTTCCGCGTTACATAACTTACG |
| | | GTAAATGGCCCGCCTGGCTGACCGCCCAACGACCCCCGCCC |
| | | ATTGACGTCAATAATGACGTATGTTCCCATAGTAACGCCAAT |
| | | AGGGACTTTCCATTGACGTCAATGGGTGGAGTATTTACGGTA |
| | | AACTGCCCACTTGGCAGTACATCAAGTGTATCATATGCCAAG |
| | | TACGCCCCCTATTGACGTCAATGACGGTAAATGGCCCGCCTG |
| | | GCATTATGCCCAGTACATGACCTTATGGGACTTTCCTACTTG |
| | | GCAGTACATCTACGTATTAGTCATCGCTATTACCATGGTCGA |
| | | GGTGAGCCCCACGTTCTGCTTCACTCTCCCCATCTCCCCCCCC |
| | | TCCCCACCCCCAATTTTGTATTTATTTATTTTTTAATTATTTTG |
| | | TGCAGCGATGGGGGCGGGGGGGGGGGGGGGGCGCGCGCCA |
| | | GGCGGGGCGGGGCGGGGCGAGGGGGGGGGGGGGCGAGGC |
| | | GGAGAGGTGCGGCGGCAGCCAATCAGAGCGGCGCGCTCCGA |
| | | AAGTTTCCTTTTATGGCGAGGCGGCGGCGGCGGCGGCCCTAT |
| | | AAAAAGCGAAGCGCGCGGCGGGCGGGGAGTCGCTGCGACG |
| | | CTGCCTTCGCCCCGTGCCCCGCTCCGCCGCCGCCTCGCGCCG |
| | | CCCGCCCCGGCTCTGACTGACCGCGTTACTCCCACAGGTGAG |
| | | CGGGCGGGACGGCCCTTCTCCTCCGGGCTGTAATTAGCGCTT |
| | | GGTTTAATGACGGCTTGTTTCTTTTCTGTGGCTGCGTGAAAG |
| | | CCTTGAGGGGCTCCGGGAGGGCCCTTTGTGCGGGGGGAGCG |
| | | GCTCGGGGGGTGCGTGCGTGTGTGTGTGCGTGGGGAGCGCC |
| | | GCGTGCGGCTCCGCGCTGCCCGGCGGCTGTGAGCGCTGCGG |
| | | GCGCGGCGCGGGGCTTTGTGCGCTCCGCAGTGTGCGCGAGG |
| | | GGAGCGCGGCCGGGGGCGGTGCCCCGCGGTGCGGGGGGGGC |
| | | TGCGAGGGGAACAAAGGCTGCGTGCGGGGTGTGTGCGTGGG |
| | | GGGGTGAGCAGGGGGTGTGGGCGCGTCGGTCGGGCTGCAAC |
| | | CCCCCCTGCACCCCCCTCCCCGAGTTGCTGAGCACGGCCCGG |
| | | CTTCGGGTGCGGGGCTCCGTACGGGGCGTGGCGCGGGGCTC |
| | | GCCGTGCCGGGCGGGGGGTGGCGGCAGGTGGGGGTGCCGGG |
| | | CGGGGCGGGGCCGCCTCGGGCCGGGGAGGGCTCGGGGGAG |
| | | GGGCGCGGCGGCCCCCGGAGCGCCGGCGGCTGTCGAGGCGC |
| | | GGCGAGCCGCAGCCATTGCCTTTTATGGTAATCGTGCGAGAG |
| | | GGCGCAGGGACTTCCTTTGTCCCAAATCTGTGCGGAGCCGAA |
| | | ATCTGGGAGGCGCCGCCGCACCCCCTCTAGCGGGCGCGGGG |
| | | CGAAGCGGTGCGGCGCCGGCAGGAAGGAAATGGGCGGGGA |
| | | GGGCCTTCGTGCGTCGCCGCGCCGCCGTCCCCTTCTCCCTCT |
| | | CCAGCCTCGGGGCTGTCCGCGGGGGGACGGCTGCCTTCGGG |
| | | GGGGACGGGGCAGGGCGGGGTTCGGCTTCTGGCGTGTGACC |
| | | GGCGGCTCTAGAGCCTCTGCTAACCATGTTCATGCCTTCTTCT |
| | | TTTTCCTACAGCTCCTGGGCAACGTGCTGGTTATTGTGCTGTC |
| | | TCATCATTTTGGCAAAGAATT |
| PGK | 419 | GGGGTTGGGGTTGCGCCTTTTCCAAGGCAGCCCTGGGTTTGC |
| | | GCAGGGACGCGGCTGCTCTGGGCGTGGTTCCGGGAAACGCA |
| | | GCGGCGCCGACCCTGGGTCTCGCACATTCTTCACGTCCGTTC |
| | | GCAGCGTCACCCGGATCTTCGCCGCTACCCTTGTGGGCCCCC |
| | | CGGCGACGCTTCCTGCTCCGCCCCTAAGTCGGGAAGGTTCCT |
| | | TGCGGTTCGCGGCGTGCCGGACGTGACAAACGGAAGCCGCA |
| | | CGTCTCACTAGTACCCTCGCAGACGGACAGCGCCAGGGAGC |
| | | AATGGCAGCGCGCCGACCGCGATGGGCTGTGGCCAATAGCG |
| | | GCTGCTCAGCAGGGCGCGCCGAGAGCAGCGGCCGGGAAGGG |
| | | GCGGTGCGGGAGGCGGGGTGTGGGGCGGTAGTGTGGGCCCT |
| | | GTTCCTGCCCGCGCGGTGTTCCGCATTCTGCAAGCCTCCGGA |
| | | GCGCACGTCGGCAGTCGGCTCCCTCGTTGACCGAATCACCGA |
| | | CCTCTCTCCCCAG |
| EF-1a | 420 | GAATTCAGGCTCCGGTGCCCGTCAGTGGGCAGAGCGCACAT |
| | | CGCCCACAGTCCCCGAGAAGTTGGGGGGAGGGGTCGGCAAT |
| | | TGAACCGGTGCCTAGAGAAGGTGGCGCGGGGTAAACTGGGA |
| | | AAGTGATGTCGTGTACTGGCTCCGCCTTTTTCCCGAGGGTGG |
| | | GGGAGAACCGTATATAAGTGCAGTAGTCGCCGTGAACGTTC |
| | | TTTTTCGCAACGGGTTTGCCGCCAGAACACAGGTAAGTGCCG |
| | | TGTGTGGTTCCCGCGGGCCTGGCCTCTTTACGGGTTATGGCC |
| | | CTTGCGTGCCTTGAATTACTTCCACCTGGCTGCAGTACGTGA |
| | | TTCTTGATCCCGAGCTTCGGGTTGGAAGTGGGTGGGAGAGTT |
| | | CGAGGCCTTGCGCTTAAGGAGCCCCTTCGCCTCGTGCTTGAG |
| | | TTGAGGCCTGGCCTGGGCGCTGGGGCCGCCGCGTGCGAATCT |
| | | GGTGGCACCTTCGCGCCTGTCTCGCTGCTTTCGATAAGTCTCT |
| | | AGCCATTTAAAATTTTTGATGACCTGCTGCGACGCTTTTTTTC |
| | | TGGCAAGATAGTCTTGTAAATGCGGGCCAAGATCTGCACACT |
| | | GGTATTTCGGTTTTTGGGGCCGCGGGCGGCGACGGGGCCCGT |
| | | GCGTCCCAGCGCACATGTTCGGCGAGGCGGGGCCTGCGAGC |
| | | GCGGCCACCGAGAATCGGACGGGGGTAGTCTCAAGCTGGCC |
| | | GGCCTGCTCTGGTGCCTGGTCTCGCGCCGCCGTGTATCGCCC |
| | | CGCCCTGGGCGGCAAGGCTGGCCCGGTCGGCACCAGTTGCG |
| | | TGAGCGGAAAGATGGCCGCTTCCCGGCCCTGCTGCAGGGAG |
| | | CTCAAAATGGAGGACGCGGCGCTCGGGAGAGCGGGCGGGTG |
| | | AGTCACCCACACAAAGGAAAAGGGCCTTTCCGTCCTCAGCC |
| | | GTCGCTTCATGTGACTCCACGGAGTACCGGGCGCCGTCCAGG |
| | | CACCTCGATTAGTTCTCGAGCTTTTGGAGTACGTCGTCTTTAG |
| | | GTTGGGGGGAGGGGTTTTATGCGATGGAGTTTCCCCACACTG |
| | | AGTGGGTGGAGACTGAAGTTAGGCCAGCTTGGCACTTGATG |
| | | TAATTCTCCTTGGAATTTGCCCTTTTTGAGTTTGGATCTTGGT |
| | | TCATTCTCAAGCCTCAGACAGTGGTTCAAAGTTTTTTTCTTCC |
| | | ATTTCAGGTGTCGTGACATCATTTT |

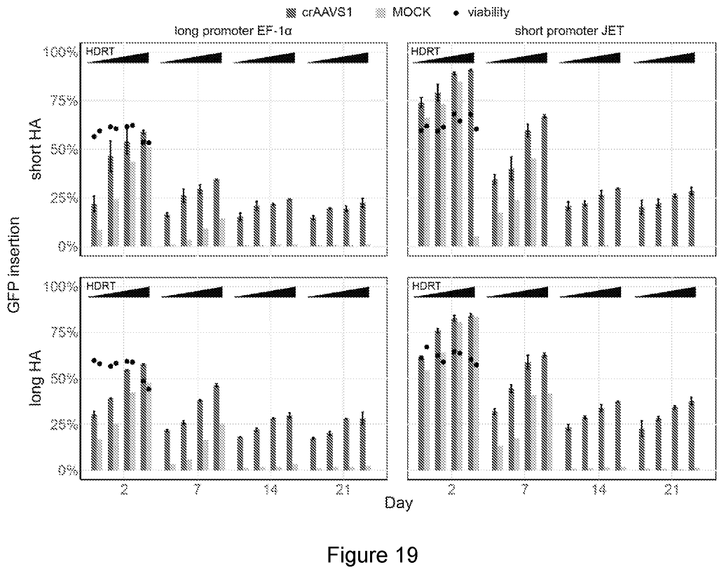

Subsequently, keeping the MAD7-RNP amounts constant, the effect of various homology arm lengths (100 vs 500 bp) and HDR template amounts (0.125 μg μL⁻¹, 0.25 μg μL⁻¹, 0.5 μg μL⁻¹, and 1 μg μL⁻¹) on the insertion efficiency was evaluated using JET and EF1a promoters. Up to 30% higher integration efficiency was observed with HDR templates flanked with HA of 500 compared to 100 base pairs. Moreover, the data showed improved insertion efficiencies with increasing amounts of HDR templates flanked with either 100 or 500 base pair HA but at the same time somewhat reduced cell viability (FIG. 19). Specifically, FIG. 19 shows GFP insertion efficiency at AAVS1 (n=3; Mean±SD) in T-cell leukemic cell line measured at days 2, 7, 14, and 21 post-transfection as a function of donor template amount. No transient GFP expression was observed at day 21 post-transfection. Cell viability (black circles) was measured at day 2 post-transfection. Top panels display GFP insertion efficiencies using donor template flanked with short homology arms (100 bp HA), and bottom panels donor template flanked with long homology arms (500 bp HA). Left panels display GFP insertion efficiencies using donor template containing EF-1a promoter (long, ˜2000 bp), and right panels donor template containing JET promoter (short, ˜1000 bp). Amount of donor template, represented by the gradient above the bars, increases from 0.125, 0.25, 0.5 to 1 μg μL⁻¹. Dark grey bars represent mean insertion frequency using crAAVS1. Light grey bars represent mean insertion frequency using crIDTneg (IDT).

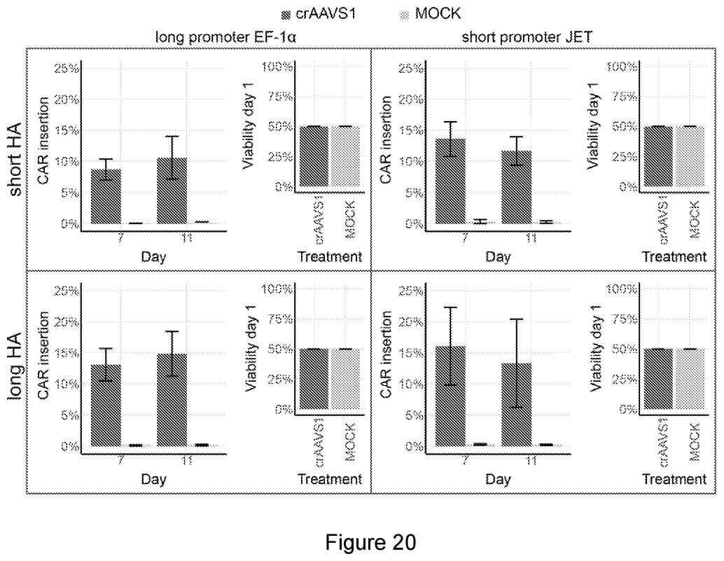

Next, using primary T-cells isolated from the human peripheral blood from three donors and a protocol selected from the experiments above, i.e., 150:100 pmol gNA:MAD7 RNP complex together with 1 μg μL⁻¹ HDR template, in combination with 100 μg μL⁻¹ poly-L-glutamic acid (PGA), integration efficiency of a clinically relevant CAR transgene containing JET or EF1a promoter flanked with HA of 100 or 500 base pairs and a bovine growth hormone derived polyadenylation sequence was analyzed. An anti-CD19 CAR with fully human variable regions (Hu19CAR), CD8a hinge and transmembrane domains, a CD28 costimulatory domain, and CD3 activation domain was used. Moderate insertion efficiency at AAVS1 but stable CAR expression of up to 14% and 16% was observed using HDR templates flanked with 100 and 500 base pair HA, respectively. The normalized cell viability measured 24 h post-transfection was in same cases relatively low, ranging from 22% with JET-500-CAR, 35% with JET-100-CAR, 43% with EF1a-100-CAR, to 55% with EF1a-500-CAR (FIG. 20). It is important to emphasize, that both CAR insertion efficiency and cell viability were higher in the treatment with PGA compared to the treatment without PGA (P≤0.05; data not shown). Specifically, FIG. 20 shows CAR insertion efficiency at AAVS1 (D=3; n=3; Mean±SD) in primary Pan T-cells measured at days 7 and 11 post-transfection. Cell viability was measured 24 hours post-transfection. Individual panels display CAR insertion efficiencies using donor template structure as described in FIG. 19. Amount of donor template, MAD7-RNP, and PGA was 1 μg μL⁻¹, 100:150 pmol MAD7:gNA, and 100 μg μL⁻¹, in that order. Nucleofection program P3-EH-115 for transfection of primary T-cells was used. D represents number of biological replicas, and n number of technical replicas per D. Dark grey bars represent mean insertion frequency using crAAVS1. Light grey bars represent mean insertion frequency using crIDTneg (IDT).

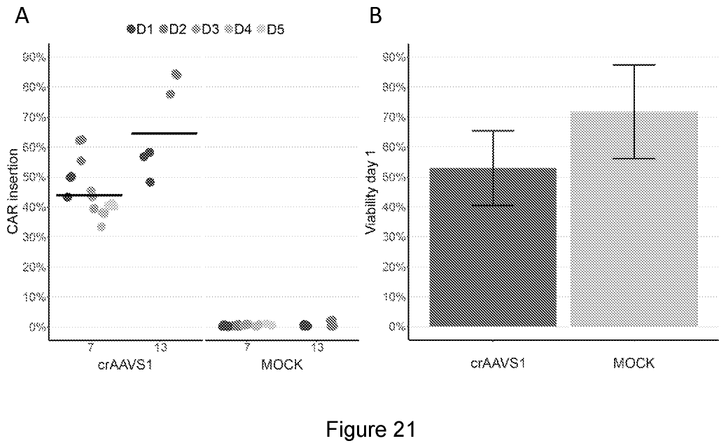

Multiple parameters were reevaluated to further optimize primary T-cell viability and CAR insertion efficiencies at AAVS1. Using Pan T-cells isolated from the blood from two donors, the effect of RNP amount with 100 μg μL⁻¹ PGA and EF1a-500-CAR template amount on CAR insertion efficiency and cell viability was tested (data not shown). Reducing the RNP amount to 75:50 pmol gNA:MAD7 RNP complex while increasing the donor template amount to 1.5 μg μL⁻¹ led to improved CAR insertion efficiencies without significantly affecting cell viability (P>0.05; data not shown). In addition, using the abovementioned transfection conditions in combination with the cell recovery in a post-transfection cultivation medium pretreated with 2 μM M3814 resulted in nearly 5-times more efficient CAR insertion than other experiments (FIG. 21). The optimized CRISPR-MAD7 transfection protocol resulted in CAR insertion efficiency of up to 85% 13-days post-transfection (median 65%) together with the median normalized cell viability as high as 62% 24 hours post-transfection. Specifically, FIG. 21 shows CAR insertion efficiency at AAVS1 (D=5; n=3) in primary Pan T-cells measured at day 7 post-transfection, and re-measured in two biological replicas at day 13 post-transfection (D=2; n=3). Cell viability was measured 24 hours post-transfection (D=5; n=3; Mean±SD). Amount or concentration of donor template, MAD7-RNP, PGA, and M3814 was 1.5 μg μL⁻¹, 50:75 pmol MAD7:gNA, 100 μg μL⁻¹, and 2 μM, respectively. Nucleofection program P3-EH-115 for transfection of primary T-cells was used. D represents number of biological replicas, and n number of technical replicas per D. Dark grey bars represent mean insertion frequency using crAAVS1. Light grey bars represent mean insertion frequency using crIDTneg (IDT).

### EQUIVALENTS

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### EMBODIMENTS

In embodiment 1 provided herein is a composition comprising a nucleic acid-guided nuclease comprising a Type V CRISPR nuclease polypeptide comprising at least one nuclear localization signal (NLS) at or near the N-terminus or the C-terminus of the polypeptide. In embodiment 2 provided herein is the composition of embodiment 1 wherein the nuclease is a Type Va nuclease. In embodiment 3 provided herein is the composition of embodiment 1 or embodiment 2 wherein the Type V CRISPR nuclease polypeptide has at least 60, 70, 80, 85, 90, 95, 96, 97, 98, 99, or 100% sequence identity, preferably at least 80%, more preferably at least 90%, even more preferably at least 95%, still more preferably at least 98% sequence identity with SEQ ID NO: 1. In embodiment 4 provided herein is the composition of any previous embodiment wherein the Type V CRISPR nuclease polypeptide comprises two NLSs, one or both of which are at or near the N-terminus or the C-terminus of the polypeptide. In embodiment 5 provided herein is the composition of any previous embodiment wherein the Type V CRISPR nuclease polypeptide comprises three NLSs, each of which is at or near the N-terminus or the C-terminus of the polypeptide. In embodiment 6 provided herein is the composition of any previous embodiment wherein the Type V CRISPR nuclease polypeptide comprises four NLSs, each of which is at or near the N-terminus or the C-terminus of the polypeptide. In embodiment 7 provided herein is the composition of any previous embodiment wherein the Type V CRISPR nuclease polypeptide comprises at least five NLSs, each of which is at or near the N-terminus or the C-terminus of the polypeptide. In embodiment 8 provided herein is the composition of any one of embodiments 4 through 7 wherein at least two of the NLSs are at or near the N-terminus of the polypeptide. In embodiment 9 provided herein is the composition of any one of embodiments 5 through 7 wherein at least three of the NLSs are at or near the N-terminus of the polypeptide. In embodiment 10 provided herein is the composition of any one of embodiments 6 through 7 wherein at least four of the NLSs are at or near the N-terminus of the polypeptide. In embodiment 11 provided herein is the composition of embodiment 7 wherein the 5 NLSs are at or near the N-terminus of the polypeptide. In embodiment 12 provided herein is the composition of embodiment 11 comprising a sequence at least 60, 70, 80, 85, 90, 95, 98, 99%, or 100%, identical, preferably at least 80%, more preferably at least 90%, even more preferably at least 95%, still more preferably at least 98% identical to any one of SEQ ID NOs: 109-112. In embodiment 13 provided herein is the composition of any one of embodiments 1 through 3 wherein the Type V CRISPR nuclease polypeptide comprises at least 1-30, 1-20, 1-15, 1-10, 1-9, 1-8, 1-7, 1-6, 1-5, 2-30, 2-20, 2-15, 2-10, 2-9, 2-8, 2-7, 2-6, 2-5, 3-30, 3-20, 3-15, 3-10, 3-9, 3-8, 3-7, 3-6, or 3-5, preferably 1-10, more preferably 2-10, even more preferably 3-10 NLSs, each of which is at or near the N-terminus or the C-terminus of the polypeptide. In embodiment 14 provided herein is the composition of any one of embodiments 4 through 11 wherein at least two of the NLSs have different nuclear localization mechanisms. In embodiment 15 provided herein is the composition of any one of embodiments 5 through 7 or 9 through 11 wherein at least three of the NLSs have different nuclear localization mechanisms. In embodiment 16 provided herein is the composition of any previous embodiment wherein one or more of the NLSs comprises an NLS of the SV40 virus large T-antigen, an NLS from nucleoplasmin, e.g. a nucleoplasmin bipartite NLS, a c-myc NLS; a hRNPA1 M9 NLS; an IBB domain of importin-alpha NLS; a myoma T protein NLS; a sequence from human p53 NLS; a sequence of mouse c-abl IV NLS; a sequence of influenza virus NS1 NLS; a sequence of Hepatitis virus delta antigen NLS; a sequence of mouse Mx1 protein NLS; a sequence of human poly(ADP-ribose) polymerase NLS; a sequence of steroid hormone receptors (human) glucocorticoid NLS; and/or a sequence of EGL-13 NLS. In embodiment 17 provided herein is the composition of embodiment 16 wherein one or more of the NLSs comprises an NLS of the SV40 virus large T-antigen. In embodiment 18 provided herein is the composition of embodiment 16 wherein two or more of the NLSs comprises an NLS of the SV40 virus large T-antigen. In embodiment 19 provided herein is the composition of embodiment 17 or embodiment 18 wherein the NLS or NLSs comprises the sequence of SEQ ID NO: 5. In embodiment 20 provided herein is the composition of any one of embodiments 16 through 19 wherein one or more of the NLSs comprises an NLS from nucleoplasmin. In embodiment 21 provided herein is the composition of embodiment 20 wherein the nucleoplasmin NLS comprises the sequence of SEQ ID NO: 6. In embodiment 22 provided herein is the composition of any one of embodiments 16 through 21 wherein one or more of the NLSs comprises a c-myc NLS. In embodiment 23 provided herein is the composition of embodiment 22 wherein the c-myc NLS comprises the sequence of SEQ ID NO: 7, SEQ ID NO: 8, or SEQ ID NO: 21. In embodiment 24 provided herein is the composition of embodiment 23 wherein the c-myc NLS comprises the sequence of SEQ ID NO: 21. In embodiment 25 provided herein is the composition of any one of embodiments 16 through 24 wherein one or more of the NLSs comprises a sequence of EGL-13 NLS. In embodiment 26 provided herein is the composition of embodiment 25 wherein the EGL-13 NLS comprises the sequence of SEQ ID NO: 107. In embodiment 27 provided herein is the composition of any previous embodiment wherein the Type V CRISPR nuclease polypeptide further comprises a purification tag. In embodiment 28 provided herein is the composition of embodiment 27 wherein the purification tag is at or near the N-terminus of the nuclease polypeptide. In embodiment 29 provided herein is the composition of embodiment 27 or embodiment 28 wherein the purification tag comprises a poly-his tag, such as a Gly-6×His tag (SEQ ID NO: 421) or Gly-8×His tag (SEQ ID NO: 422); short epitope tags, e.g., FLAG, hemagglutinin (HA), c-myc, T7, Glu-Glu; maltose binding protein (mbp); N-terminal glutathione S-transferase (GST); or calmodulin binding peptide (CBP) In embodiment 30 provided herein is the composition of embodiment 29 wherein the purification tag comprises a poly-his tag. In embodiment 31 provided herein is the composition of embodiment 30 wherein the purification tag comprises a gly-6×His tag (SEQ ID NO: 421). In embodiment 32 provided herein is the composition of embodiment 30 wherein the purification tag comprises a gly-8×His tag (SEQ ID NO: 422). In embodiment 33 provided herein is the composition of any previous embodiment wherein the Type V CRISPR nuclease polypeptide comprises a cleavage site. In embodiment 34 provided herein is the composition of embodiment 33 wherein the cleavage site is at or near the N-terminus of the nuclease polypeptide. In embodiment 35 provided herein is the composition of embodiment 33 or embodiment 34 wherein the cleavage site comprises a Tobacco Etch Virus (TEV) cleavage site. In embodiment 36 provided herein is the composition of embodiment 35 wherein the cleavage site comprises the sequence of SEQ ID NO: 108. In embodiment 37 provided herein is the composition of embodiment 36 comprising 5 NLSs at or near the N-terminus of the polypeptide, a purification tag, and the cleavage site, wherein the cleavage site is after the purification tag. In embodiment 38 provided herein is the composition of embodiment 37 comprising a sequence at least 60, 70, 80, 85, 90, 95, 98, 99%, or 100%, identical, preferably at least 8%, more preferably at least 90%, even more preferably at least 95%, still more preferably at least 98% identical to SEQ ID NO: 111 or 112. In embodiment 39 provided herein is the composition of embodiment 37 comprising a sequence at least 60, 70, 80, 85, 90, 95, 98, 99%, or 100%, identical, preferably at least 8%, more preferably at least 90%, even more preferably at least 95%, still more preferably at least 98% identical to SEQ ID NO: 112. In embodiment 40 provided herein is the composition of any previous embodiment further comprising a guide nucleic acid (gNA), e.g., gRNA, comprising a spacer sequence that targets a target nucleotide sequence within a polynucleotide, or a polynuclotide coding for the gNA, e.g., gRNA, wherein the gNA, e.g., gRNA is compatible with the Type V CRISPR nuclease. In embodiment 41 provided herein is the composition of embodiment 40 wherein the target nucleotide is within 50 nucleotides of a protospacer adjacent motif (PAM) sequence specific for the Type V CRISPR nuclease. In embodiment 42 provided herein is the composition of embodiment 41 wherein the PAM comprises a sequence of YTTN, wherein Y is T or C and Nis A, T, G, or C. In embodiment 43 provided herein is the composition of embodiment 42 wherein the PAM comprises a sequence of YTTV or TTTV, wherein V is A, G, or C. In embodiment 44 provided herein is the composition of embodiment 40 wherein the gNA is a gRNA. In embodiment 45 provided herein is the composition of embodiment 44 wherein the gRNA is a dual gRNA. In embodiment 46 provided herein is the composition of embodiment 44 or embodiment 45 wherein the composition comprises the gRNA and the gRNA comprises one or more chemical modifications. In embodiment 47 provided herein is the composition of embodiment 46 wherein the chemical modification comprises a 2′-O-alkyl, a 2′-O-methyl, a phosphorothioate, a phosphonoacetate, a thiophosphonoacetate, a 2′-O-methyl-3′-phosphorothioate, a 2′-O-methyl-3′-phosphonoacetate, a 2′-O-methyl-3′-thiophosphonoacetate, a 2′-deoxy-3′-phosphonoacetate, a 2′-deoxy-3′-thiophosphonoacetate, a suitable alternative, or a combination thereof. In embodiment 48 provided herein is the composition of any one of embodiments 44 through 47 wherein a ratio of guanine:uracil in the gRNA is at least 51:49, 52:48, 53:47, 54:46, 55:45, 56:44, 57:43, 58:42, 59:42, or 60:40, preferably at least 53:47, more preferably at least 54:46, even more preferably at least 55:45. In embodiment 49 provided herein is the composition of any one of embodiments 40 through 48 wherein the molar ratio of gNA, e.g., gRNA to Type V CRISPR nuclease is at least 1.1:1, 1.2:1, 1.3:1, 1.4:1, 1.5:1, 1.6:1, 1.7:1, 1.8:1, 2:1, 2.2:1, 2.5:1, or 3:1 and/or not more than 1.2:1, 1.3:1, 1.4:1, 1.5:1, 1.6:1, 1.7:1, 1.8:1, 2:1, 2.2:1, 2.5:1, 3:1, or 4:1, preferably 1.1:1 to 2.5:1, more preferably 1.2:1 to 2:1, even more preferably 1.2:1 to 1.7:1. In embodiment 50 provided herein is the composition of any one of embodiments 40 through 49 wherein the molar amount of gNA, e.g., gRNA, is at least 10, 25, 30, 35, 40, 45, 50, 55, 60, 65, 70, 75, 80, 85, 90, 95, 100, 110, 120, 130, 140, 150, 170, 190 or 200 pmol and/or not more than 30, 35, 40, 45, 50, 55, 60, 65, 70, 75, 80, 85, 90, 95, 100, 110, 120, 130, 140, 150, 170, 190, 200, 250, or 300 pmol, preferably 25-200 pmol, more preferably 50-100 pmol, even more preferably 65 to 85 pmol. In embodiment 51 provided herein is the composition of any one of embodiments 40 through 50 further comprising a donor template. In embodiment 52 provided herein is the composition of embodiment 51 wherein the donor template comprises homology arms. In embodiment 53 provided herein is the composition of embodiment 51 or embodiment 52 wherein the donor template is present in an amount of at least 0.1, 0.2, 0.3, 0.4, 0.5, 0.6, 0.7, 0.8, 0.9, 1, 1.1, 1.2, 1.3, 1.4, 1.5, 1.7, 2, 2.5, 3, 4, or 5 μg μL−1 and/or not more than 0.2, 0.3, 0.4, 0.5, 0.6, 0.7, 0.8, 0.9, 1, 1.1, 1.2, 1.3, 1.4, 1.5, 1.7, 2, 2.5, 3, 4, 5, 7, or 10 μg μL−1, preferably 0.3 to 2 μg μL−1, more preferably 0.5 to 1.5 μg μL−1, even more preferably 0.8 to 1.2 μg μL−1. In embodiment 54 provided herein is the composition of any one of embodiments 40 through 53 further comprising an anionic polymer. In embodiment 55 provided herein is the composition of embodiment 54 wherein the anionic polymer comprises polyglutamic acid (PGA). In embodiment 56 provided herein is the composition of embodiment 54 or embodiment 55 wherein the anionic polymer is present at a concentration of at least 20, 50, 60, 70, 80, 90, 100, 110, 120, 130, 140, 150, 170, 200, 250, 300, 400, or 500 μg μL−1 and/or not more than 50, 60, 70, 80, 90, 100, 110, 120, 130, 140, 150, 170, 200, 250, 300, 400, 500, 700, or 1000 μg μL−1, preferably 20 to 200 μg μL−1, more preferably 50 to 150 μg μL−1, even more preferably 80 to 120 μg μL−1 .

In embodiment 57 provided herein is a cell containing the composition of any previous embodiment. In embodiment 58 provided herein is the cell of embodiment 56 wherein the cell is a human cell. In embodiment 59 provided herein is the cell of embodiment 58 wherein the cell is an immune cell or a stem cell. In embodiment 60 provided herein is the cell of embodiment 59 wherein the cell is an immune cell. In embodiment 61 provided herein is the cell of embodiment 60 wherein the cell is a T cell. In embodiment 62 provided herein is the cell of embodiment 59 wherein the cell is a stem cell. In embodiment 63 provided herein is the cell of embodiment 62 wherein the cell is an induced pluripotent stem cell (iPSC).

In embodiment 64 provided herein is a method comprising inserting a composition of any one of embodiments 1 through 56 into a cell. In embodiment 65 provided herein is the method of embodiment 64 wherein inserting the composition into the cell comprises electroporation.

In embodiment 66 provided herein is a method for modifying a target polynucleotide comprising (i) contacting the composition of any one of embodiments 40 through 56 and (ii) allowing the nuclease and the guide nucleic acid to modify a targeted genomic region. In embodiment 67 provided herein is the method of embodiment 66 wherein the composition is a composition of any one of embodiments 51 through 56. In embodiment 68 provided herein is the method of embodiment 66 or embodiment 67 wherein the target polynucleotide is a genome or a portion of a genome within a cell. In embodiment 69 provided herein is the method of embodiment 68 wherein the cell is a human cell. In embodiment 70 provided herein is the method of embodiment 69 wherein the cell is an immune cell or a stem cell. In embodiment 71 provided herein is the method of embodiment 70 wherein the cell is an immune cell. In embodiment 72 provided herein is the method of embodiment 71 wherein the cell is a T cell. In embodiment 73 provided herein is the method of embodiment 70 wherein the cell is a stem cell. In embodiment 74 provided herein is the method of embodiment 73 wherein the stem cell is an iPSC In embodiment 75 provided herein is the method of any one of embodiments 67 through 74 wherein the donor template comprises a mutation in a PAM within 50 nucleotides of the target nucleotide sequence in the target polynucleotide. In embodiment 76 provided herein is the method of any one of embodiments 68 through 74 wherein the composition is a composition of embodiment 67 and the donor template comprises a polynucleotide coding for a polypeptide to be expressed by the cell. In embodiment 77 provided herein is the method of embodiment 76 wherein the polypeptide to be expressed by the cell comprises a chimeric antigen receptor (CAR) or a portion thereof. In embodiment 78 provided herein is the method of embodiment 77 wherein the cell is a human T cell or a human iPSC. In embodiment 79 provided herein is the method of embodiment 77 wherein the cell is a human T cell. In embodiment 80 provided herein is the method of embodiment 77 wherein the cell is a human iPSC.

In embodiment 81 provided herein is a composition comprising a first polynucleotide coding for a polypeptide comprising a nucleic acid-guided nuclease comprising a CRISPR Type V nuclease polypeptide, wherein the polynucleotide has less than 75% sequence identity to SEQ ID NO: 22. In embodiment 82 provided herein is the composition of embodiment 81 wherein the nuclease polypeptide comprises at least 1, 2, 3, 4, or 5 NLSs, wherein each of the NLSs is at or near the N-terminus or the C-terminus of the nuclease polypeptide. In embodiment 83 provided herein is the composition of embodiment 82 wherein one or more of the NLSs comprises an NLS of the SV40 virus large T-antigen, an NLS from nucleoplasmin, e.g. a nucleoplasmin bipartite NLS, a c-myc NLS; a hRNPA1 M9 NLS; an IBB domain of importin-alpha NLS; a myoma T protein NLS; a sequence from human p53 NLS; a sequence of mouse c-abl IV NLS; a sequence of influenza virus NS1 NLS; a sequence of Hepatitis virus delta antigen NLS; a sequence of mouse Mx1 protein NLS; a sequence of human poly(ADP-ribose) polymerase NLS; a sequence of steroid hormone receptors (human) glucocorticoid NLS; and/or a sequence of EGL-13 NLS. In embodiment 84 provided herein is the composition of embodiment 83 wherein one or more of the NLSs comprises an NLS of the SV40 virus large T-antigen. In embodiment 85 provided herein is the composition of embodiment 84 wherein the NLS or NLSs comprises the sequence of SEQ ID NO: 5. In embodiment 86 provided herein is the composition of any one of embodiments 83 through 85 wherein one or more of the NLSs comprises an NLS from nucleoplasmin. In embodiment 87 provided herein is the composition of embodiment 86 wherein the nucleoplasmin NLS comprises the sequence of SEQ ID NO: 6. In embodiment 88 provided herein is the composition of any one of embodiments 83 through 87 wherein one or more of the NLSs comprises a c-myc NLS. In embodiment 89 provided herein is the composition of embodiment 88 wherein the c-myc NLS comprises the sequence of SEQ ID NO: 7, SEQ ID NO: 8, or SEQ ID NO: 21. In embodiment 90 provided herein is the composition of embodiment 88 wherein the c-myc NLS comprises the sequence SEQ ID NO: 21. In embodiment 91 provided herein is the composition of any one of embodiments 83 through 90 wherein one or more of the NLSs comprises a sequence of EGL-13 NLS. In embodiment 92 provided herein is the composition of embodiment 91 wherein the EGL-13 NLS comprises the sequence of SEQ ID NO: 107. In embodiment 93 provided herein is the composition of any one of embodiments 82 through 92 wherein the NLS or NLSs is at or near the N-terminus of the polypeptide. In embodiment 94 provided herein is the composition of any one of embodiments 81 through 93 wherein the first polynucleotide comprises a polynucleotide coding for a purification tag. In embodiment 95 provided herein is the composition of embodiment 94 wherein the purification tag is at or near the N-terminus of the nuclease polypeptide. In embodiment 96 provided herein is the composition of embodiment 94 or 95 wherein the purification tag comprises a poly-his tag, such as a Gly-6×His tag (SEQ ID NO: 421) or Gly-8×His tag (SEQ ID NO: 422); short epitope tags, e.g., FLAG, hemagglutinin (HA), c-myc, T7, Glu-Glu; maltose binding protein (mbp); N-terminal glutathione S-transferase (GST); or calmodulin binding peptide (CBP). In embodiment 97 provided herein is the composition of embodiment 96 wherein the purification tag comprises a poly-his tag. In embodiment 98 provided herein is the composition of embodiment 97 wherein the purification tag comprises a gly-6×His tag (SEQ ID NO: 421). In embodiment 99 provided herein is the composition of embodiment 97 wherein the purification tag comprises a gly-8×His tag (SEQ ID NO: 422). In embodiment 100 provided herein is the composition of any one of embodiments 81 through 99 wherein the Type V CRISPR nuclease polypeptide comprises a cleavage site. In embodiment 101 provided herein is the composition of embodiment 100 wherein the cleavage site is at or near the N-terminus of the nuclease polypeptide. In embodiment 102 provided herein is the composition of embodiment 100 or 101 wherein the cleavage site comprises a Tobacco Etch Virus (TEV) cleavage site. In embodiment 103 provided herein is the composition of embodiment 102 wherein the cleavage site comprises the sequence of SEQ ID NO: 108. In embodiment 104 provided herein is the composition of embodiment 103 comprising 5 NLSs at or near the N-terminus of the polypeptide, a purification tag, and the cleavage site, wherein the cleavage site is after the purification tag. In embodiment 105 provided herein is the composition of any one of embodiments 81 through 104 wherein the polynucleotide codes for a polypeptide comprising a sequence at least 60, 70, 80, 85, 90, 95, 98, 99%, or 100%, identical, preferably at least 80%, more preferably at least 90%, even more preferably at least 95%, still more preferably at least 98% identical to any one of SEQ ID NOs: 109-112 In embodiment 106 provided herein is the composition of any one of embodiments 81 through 105 wherein the polynucleotide codes for a polypeptide comprising a sequence at least 60, 70, 80, 85, 90, 95, 98, 99%, or 100%, preferably at least 80%, more preferably at least 90%, even more preferably at least 95%, still more preferably at least 98% identical identical to SEQ ID NO: 112. In embodiment 107 provided herein is the composition of any one of embodiments 81 through 105 wherein the first polynucleotide comprises a sequence at least 50, 60, 70, 80, 90, 95, 97, or 99% identical, or 100% identical, preferably at least 80%, more preferably at least 90%, even more preferably at least 95%, still more preferably at least 98% identical to SEQ ID NO: 113. In embodiment 108 provided herein is the composition of any one of embodiments 81 through 107 further comprising a second polynucleotide coding for a gNA or portion thereof, wherein the gNA, e.g., gRNA, comprises a spacer sequence that targets a target nucleotide sequence within a polynucleotide, or a polynuclotide coding for the gNA, e.g., gRNA, wherein the gNA, e.g., gRNA is compatible with the Type V CRISPR nuclease. In embodiment 109 provided herein is the composition of embodiment 108 wherein the first and second polynucleotides are the same. In embodiment 110 provided herein is the composition of any one of embodiments 81 through 109 further comprising third polynucleotide that comprises a donor template.

In embodiment 111 provided herein is a vector comprising the polynucleotide or polynucleotides of any one of embodiments 81 through 110.

In embodiment 112 provided herein is a cell comprising a composition of any one of embodiments 81 through 110. In embodiment 113 provided herein is the composition of embodiment 112 wherein the cell is a human cell. In embodiment 114 provided herein is the composition of embodiment 113 wherein the cell is an immune cell or a stem cell. In embodiment 115 provided herein is the composition of embodiment 113 wherein the cell is an immune cell. In embodiment 116 provided herein is the composition of embodiment 115 wherein the cell is T cell. In embodiment 117 provided herein is the composition of embodiment 113 wherein the cell is a stem cell. In embodiment 118 provided herein is the composition of embodiment 117 wherein the cell is an iPSC.

In embodiment 119 provided herein is a method comprising inserting the composition of any one of embodiments 81 through 111 into a cell. In embodiment 120 provided herein is the method of embodiment 119 wherein inserting the composition into the cell comprises electroporation.

In embodiment 121 provided herein is a method comprising (i) inserting a composition of any one of embodiments 81 through 107 into a cell and (ii) inserting a gNA, e.g. a gRNA, compatible with the Type V CRISPR nuclease coded for by the composition, into the cell. In embodiment 122 provided herein is the method of embodiment 121 wherein steps (i) and (ii) comprise electroporation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-122. (canceled)

123. A composition comprising a nucleic acid-guided nuclease comprising a Type V CRISPR nuclease polypeptide comprising at least three nuclear localization signals (NLS) at or near the N-terminus or the C-terminus of the polypeptide, wherein at least two of the NLSs are at or near the N-terminus of the polypeptide, or a polynucleotide encoding the nuclease.

124. The composition of claim 123 wherein the nuclease is a Type Va nuclease.

125. The composition of claim 123 wherein the Type V CRISPR nuclease polypeptide has at least 80% sequence identity with SEQ ID NO: 1.

126. The composition of claim 123 wherein the Type V CRISPR nuclease polypeptide comprises four NLSs, each of which is at or near the N-terminus or the C-terminus of the polypeptide.

127. The composition of claim 123 wherein the Type V CRISPR nuclease polypeptide comprises at least five NLSs, each of which is at or near the N-terminus or the C-terminus of the polypeptide.

128. The composition of claim 123 wherein at least three of the NLSs are at or near the N-terminus of the polypeptide.

129. The composition of claim 126 wherein at least four of the NLSs are at or near the N-terminus of the polypeptide.

130. The composition of claim 127 wherein the 5 NLSs are at or near the N-terminus of the polypeptide.

131. The composition of claim 130 wherein the polypeptide comprises a sequence at least 80%, identical to any one of SEQ ID NOs: 109-112.

132. The composition of claim 123 wherein at least two of the NLSs have different nuclear localization mechanisms.

133. The composition of claim 123 wherein one or more of the NLSs comprises an NLS of the SV40 virus large T-antigen, an NLS from nucleoplasmin, e.g. a nucleoplasmin bipartite NLS, a c-myc NLS; a hRNPA1 M9 NLS; an IBB domain of importin-alpha NLS; a myoma T protein NLS; a sequence from human p53 NLS; a sequence of mouse c-abl IV NLS; a sequence of influenza virus NS1 NLS; a sequence of Hepatitis virus delta antigen NLS; a sequence of mouse Mx1 protein NLS; a sequence of human poly(ADP-ribose) polymerase NLS; a sequence of steroid hormone receptors (human) glucocorticoid NLS; and/or a sequence of EGL-13 NLS.

134. The composition of claim 123 wherein the Type V CRISPR nuclease polypeptide further comprises a purification tag at or near the N-terminus of the nuclease polypeptide.

135. The composition claim 134 wherein the Type V CRISPR nuclease polypeptide comprises a cleavage site at or near the N-terminus of the nuclease polypeptide.

136. The composition of claim 135 comprising 5 NLSs at or near the N-terminus of the polypeptide, a purification tag, and the cleavage site, wherein the cleavage site is after the purification tag.

137. The composition of claim 136 comprising a sequence at least 80%, identical to SEQ ID NO: 111 or 112.

138. The composition of claim 123 further comprising a guide nucleic acid (gNA), comprising a spacer sequence that targets a target nucleotide sequence within a polynucleotide, or a polynuclotide coding for the gNA, wherein the gNA, is compatible with the Type V CRISPR nuclease.

139. The composition of claim 138 wherein the gRNA is a dual gRNA.
